(19) United States
(12) Patent Application Publication
(10) Pub. No.: US 2025/0179481 A1
(43) Pub. Date: Jun. 5, 2025

(54) COMPOSITIONS AND METHODS FOR TARGETING, EDITING, OR MODIFYING GENES

(71) Applicant: Celyntra Therapeutics SA, Mont-Saint-Guibert (BE)

(72) Inventors: Roland BAUMGARTNER, Angern an der March (AT); Tanya WARNECKE, Boulder, CO (US)

(21) Appl. No.: 18/566,506

(22) PCT Filed: Jun. 1, 2022
(86) PCT No.: PCT/US2022/031835

§ 371 Date: Dec. 1, 2023

Related U.S. Application Data
(60) Provisional application No. 63/195,615, filed on Jun. 1, 2021.

(51) Int. Cl. C12N 15/11; C12N 9/22; C12N 15/90

(52) U.S. Cl. CPC C12N 15/11; C12N 9/22; C12N 15/907; C12N 2310/20; C12N 2800/80

(57) ABSTRACT

CRISPR-Cas systems have been engineered for various purposes, such as genomic DNA cleavage, base editing, epigenome editing, and genomic imaging. Although significant developments have been made, there still remains a need for new and useful CRISPR-Cas systems as powerful precise genome targeting tools. The invention disclosed herein comprises CRISPR-Cas based methods for high integration and expression efficiency of transgenes together with high post-transfection cell viability in eukaryotic cells.

This application claims the benefit of U.S. Provisional Application No. 63/195,615 filed Jun. 1, 2021, which application is incorporated herein by reference.

### BACKGROUND

Recent advances have been made in precise genome targeting technologies. For example, specific loci in genomic DNA can be targeted, edited, or otherwise modified by designer meganucleases, zinc finger nucleases, or transcription activator-like effectors (TALEs). Furthermore, the CRISPR-Cas systems of bacterial and archaeal adaptive immunity have been adapted for precise targeting of genomic DNA in eukaryotic cells. Compared to the earlier generations of genome editing tools, the CRISPR-Cas systems are easy to set up, scalable, and amenable to targeting multiple positions within the eukaryotic genome, thereby providing a major resource for new applications in genome engineering.

Two distinct classes of CRISPR-Cas systems have been identified. Class 1 CRISPR-Cas systems utilize multi-protein effector complexes, whereas class 2 CRISPR-Cas systems utilize single-protein effectors. Among the three types of class 2 CRISPR-Cas systems, type II and type V systems typically target DNA and type VI systems typically target RNA. Naturally occurring type II effector complexes consist of Cas9, CRISPR RNA (crRNA), and trans-activating CRISPR RNA (tracrRNA), but the crRNA and tracrRNA can be fused as a single guide RNA in an engineered system for simplicity. Certain naturally occurring type V systems, such as type V-A, type V-C, and type V-D systems, do not require tracrRNA and use crRNA alone as the guide for cleavage of target DNA.

The CRISPR-Cas systems have been engineered for various purposes, such as genomic DNA cleavage, base editing, epigenome editing, and genomic imaging. Although significant developments have been made, there still remains a need for new and useful CRISPR-Cas systems as powerful precise genome targeting tools. In CRISPR-Cas systems, a Cas nuclease is targeted to a genomic site by complexing with a guide RNA that hybridizes to a target site in the genome. This results in a double-strand break that initiates either non-homologous end-joining (NHEJ) or homology-directed repair (HDR) of genomic DNA via a double-strand or single-strand DNA repair template. However, repair of a genomic site via HDR is inefficient. In addition, off-target binding and double strand breaks can lead to undesired alterations in the genome.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A shows a schematic representation showing the structure of an exemplary single guide type V-A CRISPR system. FIG. 1B is a schematic representation showing the structure of an exemplary dual guide type V-A CRISPR system.

FIGS. 2A-C show a series of schematic representations showing incorporation of a protecting group (e.g., a protective nucleotide sequence or a chemical modification) (FIG. 2A), a donor template-recruiting sequence (FIG. 2B), and an editing enhancer (FIG. 2C) into a type V-A CRISPR-Cas system. These additional elements are shown in the context of a dual guide type V-A CRISPR system, but it is understood that they can also be present in other CRISPR systems, including a single guide type V-A CRISPR system, a single guide type II CRISPR system, or a dual guide type II CRISPR system.

FIG. 3 shows a schematic of a Type V-A nucleic acid guide nuclease bound to a dual guide nucleic acid.

FIG. 4 shows exemplary MAD7s with one or more nuclear localization signals (NLS).

FIG. 5 shows editing frequency at the DNMT1 locus in and post-transfection cell viability of T-cell leukemic cells following treatment with one or more guide nucleic acids complexed with MAD7 comprising one or more NLS.

FIG. 6 shows editing frequency at the DNMT1 locus in T-cell leukemic cells using multiple electroporation programs in combination with the SE electroporation buffer.

FIG. 7 shows editing frequency at the DNMT1 locus in T-cell leukemic cells using multiple electroporation programs in combination with the SF electroporation buffer.

FIG. 8 shows editing frequency at the DNMT1 locus in T-cell leukemic cells using multiple electroporation programs in combination with the SG electroporation buffer.

FIG. 9 shows editing frequency at the DNMT1 locus in T-cell leukemic cells using multiple electroporation programs.

FIG. 10 shows editing frequency by type at eight loci in T-cell leukemic cells using multiple guide nucleic acids complexed with MAD7 comprising one or more NLS.

FIG. 11 shows a comparison of editing efficiency between T-cell leukemic cells treated with MAD7 comprising one or more guide nucleic acids targeting the DNMT1 locus as compared to a control guide nucleic acid binned by editing frequency.

FIG. 12 shows editing frequency by PAM motif in T-cell leukemic cells using multiple guide nucleic acids complexed with MAD7 comprising one or more NLS.

FIG. 13A shows sequence logo plots for multiple guide nucleic acids binned by editing frequency in T-cell leukemic cells using when complexed with MAD7 comprising one or more NLS.

FIG. 13B shows nucleotide and dinucleotide frequency for multiple guide nucleic acids binned by editing frequency in T-cell leukemic cells using when complexed with MAD7 comprising one or more NLS.

FIG. 14 shows trinucleotide AAA or UUU frequency binned by editing frequency in T-cell leukemic cells following treatment with multiple guide nucleic acids complexed with MAD7 comprising one or more NLS.

FIG. 15 shows editing frequency for both INDELs and frameshift mutations at eight loci in T-cell leukemic cells following treatment with multiple guide nucleic acids complexed with MAD7 comprising one or more NLS.

FIG. 16 shows the correlation between INDEL frequency in the gNA validation experiment versus INDEL formation in the gNA screen experiment.

FIG. 17 shows the proportion of frameshift to INDELs at eight loci in T-cell leukemic cells following treatment with multiple guide nucleic acids complexed with MAD7 comprising one or more NLS.

FIG. 18 shows INDEL frequency for gNAs comprising representative spacer sequences complexed with MAD7 comprising one or more NLS in T-cell leukemic cells at predicted off-target sites.

FIG. 19 shows INDEL frequency for gNAs comprising representative spacer sequences complexed with MAD7 comprising one or more NLS in T-cell leukemic cells at predicted off-target sites.

FIG. 20 shows INDEL frequency at the AAVS1 locus in T-cell leukemic cells following treatment with a gNA: MAD7 complex.

FIG. 21 shows GFP insertion efficiency at the AAVS1 locus and cell viability following treatment for multiple primer constructs.

FIG. 22 shows GFP insertion efficiency at the AAVS1 locus with increasing concentrations of donor template (e.g., HDRT) and variable homology arm length.

FIG. 23 shows CAR insertion efficiency at the AAVS1 locus and cell viability with increasing concentrations of donor template and variable homology arm length.

FIG. 24 shows CAR insertion efficiency (A) at the AAVS1 locus and cell viability (B) in primary T-cells.

FIG. 25 illustrates an exemplary method for stabilizing nucleic acid-guided nucleases.

FIG. 26 illustrates an exemplary method for engineering a taget genome, e.g., human target genome.

FIG. 27 shows data for editing efficiency (as measured by # of reads modified/total # of reads) in primary T-cells in an Exon of an exemplary gene for a series of schematic representations of exemplary modifications to dual guide gRNA. Shown are editing results relative to the single gRNA design (left bar) vs, the negative control (far right bar).

FIG. 28 shows results of tiling experiment of TRBC and CD3E guides in Jurkat cells. (A) Schematic overview of the protein coding exons of TRBC1 and TRBC2 and the location of the designed gRNAs. (B) Tiling results of the TRBC gRNAs with the resulting INDEL and Substitution frequencies. (C) Schematic overview of the protein coding exons of CD3E and the location of the designed gRNAs. (D) Tiling results of the CD3E gRNAs with the resulting INDEL and substitution frequencies.

FIG. 29 shows results of tiling experiment of CD40LG and CSF2 guides. (A) Schematic overview of the protein coding exons of CD40LG and the location of the designed gRNAs. (B) Tiling results of the CD40LG gRNAs with the resulting INDEL and substitution frequencies. (C) Schematic overview of the protein coding exons of CSF2 and the location of the designed gRNAs. (D) Tiling results of the CSF2 gRNAs with the resulting INDEL and Substitution frequencies.

FIG. 30 shows gRNA verification for multiple TRBC1 and 2 gNAs. (A) TCR staining results after transfection of TRBC1 and TRBC2 RNPs and the control. (B) Viability of the RNP transfected cells and controls at day 1 and day 4.

FIG. 31 shows CD3E gRNA verification in Jurkat cells on the genomic and functional level. (A) Amplicon NGS results after transfection of CD3E RNPs. (B) and (C) TCR and CD3E staining results after transfection of gCD3E RNPs and the controls, respectively. (D) Viability of the RNP transfected cells and controls at day 1 and day 4.

FIG. 32 shows CD40LG and CSF2 gRNA verification in Jurkat cells on a genomic level. (A) Amplicon-NGS results following transfection of Jurkat cells with gCD40LG RNPs. (B) Viability of the Jurkat cells following transfection with gCD40LG RNPs. (C) Amplicon-NGS results following transfection of Jurkat cells with gCSF2 RNPs. (D) Viability of the Jurkat cells following transfection with gCSF2 RNPs.

FIG. 33 shows cutting, editing and functional KO efficiency of TRBC1, TRBC2, CD3E, CD40LG and CSF2 in Pan T-cells. (A), (B), (D) and (F) On-target verification of the TRBC, CD3E, CD40LG and CSF2 gRNAs treated Pan T-cells using Amplicon-NGS. (C) Functional KO verification of TRBC and CD3E RNP-treated Pan T-cells of TCR and CD3E surface expression using anti-TCR and anti-CD3E antibody staining. (E) Functional KO verification of CD40LG RNP-treated Pan T-cells of CD40LG surface expression using an anti-CD40LG antibody staining. Prior to staining, cells were treated with CD3/CD28. (G) Functional KO verification of CSF2 RNP-treated Pan T-cells by CSF2 intracellular expression using an anti-CSF2 antibody staining. Prior to staining, cells were treated with PMA and Ionomycin to increase CSF2 expression and Golgi-plug/Golgi-stop were used to inhibit its secretion.

FIG. 34 shows HDR enhancer shuts down the NHEJ pathway and enhances ssODN integration.

DETAILED DESCRIPTION

Outline

| I. | ssODN compositions and methods | |
|---|---|---|
| II. | High efficiency transgene insertion | |
| III. | Engineered non-naturally-occurring dual guide CRISPR-cas systems | |
| | A. | Cas proteins |
| | B. | Guide nucleic acids |
| | C. | gNA Modifications |
| IV. | Composition and methods for targeting, editing, and/or modifying genomic DNA | |
| | A. | Ribonucleoprotein (RNP) delivery and “cas RNA” delivery |
| | B. | CRISPR expression systems |
| | C. | Donor templates |
| | D. | Efficiency and specificity |
| | E. | Multiplex |
| | F. | Genes to be modified |
| V. | Pharmaceutical compositions | |
| VI. | Therapeutic uses | |
| | A. | Gene therapies |
| | B. | Immune cell engineering |

| | |
|---|---|
| VII. | Kits |
| VIII. | Embodiments |
| IX. | Examples |
| X. | Equivalents |

I, ssODN Compositions and Methods

Provided herein are methods and compositions utilizing single stranded oligo DNA nucleotides (ssODNs) in CRISPR systems. The methods and compositions are useful in favoring homology-driven recombination (HDR), and/or in correcting off-target modifications in nucleic acid. In certain embodiments, the CRISPR system includes a Type V endonuclease. In certain embodiments, ssODNs as described herein are used with a dual guide RNA. In certain embodiments, ssODNS as described herein are used with a guide RNA, such as a dual guide RNA, wherein one or more nucleotides of the RNA is a modified nucleotide. One purpose of the methods and compositions provided herein is to improve editing specificity for CRISPR systems. Specifically, ssODNs can be used for programming a precise on-target edit for improved functional disruption and for reducing off-target editing at other sites.

It is known that ssODNs can be incorporated into a target genome via homology directed repair (HDR) to program a precise edit. Combining ssODNs with a CRISPR endonuclease editing system to create a double stranded break at the target site for incorporation is well known to increase efficiencies significantly over the wild-type HDR alone and has been the basis of many CRISPR-based applications for genome engineering wherein the nuclease is a Cas9 nuclease. In certain embodiments, provided herein are systems utilizing a Type V, e.g., Type V-A nuclease. In addition, off-target editing can be reduced. The methods and compositions provided herein can reduce off-target effects by, e.g., using ssODNs engineered to preferentially bind to off-target DNA sites and to incorporate the wild type (wt) off-target gene back into the site, so that after repair the off-target site still comprises a functional wild type gene. In addition, in certain embodiments a composition is comprised of a ssODN or pool of ssODNs that are designed to have homology arms to an on-target or potential off-target editing site and, in certain embodiments, to have an editing window that creates a deletion of or edit that includes a PAM mutations, e.g., synonymous PAM mutation at the target PAM. In addition to the PAM modification, e.g., deletion, the ssODN can include additional edits such as stop codons or other changes that could change the coding sequence. The ssODNs are used in conjunction with a guide RNA (gRNA), which can be a single gRNA (sgRNA) or dual gRNA (dgRNA), depending on the nuclease used, and a nuclease. In certain embodiments, the gRNA comprises one or more modified nucleotides. The nuclease can be any suitable nuclease. In certain embodiments, the nuclease is a Type V nuclease, such as a Type Va nuclease. In certain embodiments, the nuclease is modified to include one or more nuclear localization sequences (NLSs) and/or tags such as a gly-polyHis tag. In certain embodiments, the gRNA comprises a spacer sequence that targets a specific gene as disclosed herein. In certain embodiments, after transfection the cells are treated with an HDR enhancer, for example for 24 hours, to block the NHEJ pathway and thereby increase the incorporation of the ssODN at the on-target side. In certain embodiments, an anionic polymer is used to increase transfection efficiency.

In certain embodiments provided herein is a composition comprising a plurality of ssODNs wherein each of the ssODNs comprises (i) a sequence that is complementary to and specific for a sequence flanking a double-stranded break at an off-target site for a nucleic acid-guided nuclease complex comprising a nucleic acid-guided nuclease and a gNA, e.g., gRNA, wherein the ssODNs each comprise different sequences for different off-target sites. As used herein, the terms “nucleic acid-guided nuclease complex,” “nucleic acid-guided nuclease system,”, and the like, include a system that comprises a CRISPR nuclease and a compatible gNA, e.g., gRNA. As used herein the term “complementary” includes a sequence of sufficient complementarity to hybridize with its intended hybridization partner, under conditions in which the sequence is used, unless otherwise indicated. In certain embodiments, the composition includes the nucleic acid-guided nuclease and gNA. It will be appreciated that a composition may instead provide one or more polynucleotides coding for one or both of the nuclease and the gNA, e.g., gRNA and that cellular machinery is relied on to provide the final nuclease and/or gNA, e.g., gRNA, and such embodiments are included herein. In certain embodiments some or all of the ssODNs comprise a sequence for a wild-type gene at the off-target site. In certain embodiments, more than one nucleic acid-guided nuclease complex is provided, where each complex has a different on-target site and, potentially, the same and/or different off-target sites. The nucleic acid-guided nuclease complex or complexes may be used to inactivate one or more genes and/or to insert a heterologous gene (transgene) at its on-target site. In certain embodiments, a plurality of nucleic acid-guided nucleases is provided. On-target sites for the one or more nuclease complexes can include safe harbor sites, such as the AAV1 site or other known or suitable safe harbor sites, for example one or more safe harbor sites in intergenic DNA. On-target sites for the one or more nuclease complexes can include one or more genes involved in host-versus-graft or graft-versus host disease, such as genes coding for one or more subunits of HLA-1 or HLA-2 proteins, and/or genes coding for transcription factors for the one or more subunits, such as CIITA, and/or genes coding for one or more subunits of the TCR. A transgene, provided as part of a donor template, may be inserted at one or more of the on-target sites, such as a transgene coding for a chimeric antigen receptor (CAR) or a portion thereof. The off-target ssODNs typically will comprise homology arms for the off-target site that are more complementary to the genomic sequence at the off-target site than homology arms of an on-target ssODN. In certain embodiments, at least 5, 10, 15, 20, 30, 40, 50, 60, 70, 80, 90, 95, 99 or 100% of the ssODNs further comprise at least one mutation, e.g., synonymous mutation, to prevent re-cleavage of the non-target DNA following incorporation of the ssODN into the genome of the cell, for example, a mutation in a PAM sequence of the off-target site, e.g., a mutation that decreases or eliminates recognition of the off-target site by the nucleic acid-guided nuclease complex, such as a decrease of at least 10, 20, 30, 40, 50, 60, 70, 80, 90, 92, 95, 97, or 99% in recognition of the off-target site: it will be appreciated that without recognition there will not be cleavage at the site. In certain embodiments, use of the composition in combination with one or more on-target gRNAs allows repair of off-target cleavage sites, where the off-target ssODNs comprising a replacement wt gene at the off-target site are thought to out-compete the on-target ssODN for repair of the off-target DNA breaks, then modification of the sites so that the RNP will not recognize the repaired sites and further off-target cleavage is avoided. The composition can further comprise an on-target ssODN, that is, an ssODN that comprises (i) a sequence that is complementary to and hybridizes with a genomic sequence flanking a double-stranded break, if present, at an on-target site for a gRNA that is complexed with a Cas nuclease; and (ii) a sequence to modify the coding region at the on-target site. The modification can include one or more insertions or deletions, or changes in the native sequence. In certain embodiments, the modification can include an insertion or a deletion that creates a frame shift in the reading frame of a protein and/or a stop codon or several stop codons to truncate translation of the protein. In certain embodiments, the composition comprises at least 2, 5, 7, 10, 12, 15, 17, 20, 25, 30, 35, 40, 45, 50, 60, 70, 80, 90, 100, 120, 150, or 200 different off-target ssODNs and/or not more than 5, 7, 10, 12, 15, 17, 20, 25, 30, 35, 40, 45, 50, 60, 70, 80, 90, 100, 120, 150, 200 or 500 off-target ssODNs. The length of the ssODN may be any suitable length, for example at least 20, 50, 70, 80, 90, 100, 110, 120, 130, 140, 150, 160, 170, 180, 190, 200, 210, 220, 230, 240, 250, 270, or 300 nucleotides and/or not more than 50, 70, 80, 90, 100, 110, 120, 130, 140, 150, 160, 170, 180, 190, 200, 210, 220, 230, 240, 250, 270, 300, or 400 nucleotides preferably 100-300 nucleotides, more preferably 150-250 nucleotides, even more preferably 180-220 nucleotides. The ratio of molar amount of off-target ssODN for a given off-target site to the molar amount of on-target ssODN can be any suitable ratio: the ratio may be different for different off-target sites or the same. Exemplary ratios (single off-target ssODN: on-target ssODN) include at least 0.1:1, 0.5:1, 1:1, 1.2:1, 1.4:1, 1.6:1, 1.8:1, 2:1, 2.5:1, 3:1, 4:1, 5:1, 7:1, 10:1, 15:1, 20:1, 50:1 or 100:1 and/or not more than 0.5:1, 1:1, 1.2:1, 1.4:1, 1.6:1, 1.8:1, 2:1, 2.5:1, 3:1, 4:1, 5:1, 7:1, 10:1, 15:1, 20:1, 50:1, 100:1, or 200:1. The ratio of off-target to on-target ssODN used can be dependent on the predicted likelihood of cleavage at a given off-target site vs, cleavage at the on-target site, which can be determined by methods known in the art. Typically the ssODN or plurality of ssODNs (off-target and on-target) will be used in conjunction with a nuclease and a gNA, e.g., gRNA. The nuclease can be any suitable Cas nuclease, such as a Class 1 or Class 2 nuclease, e.g., Type I, II, III, IV, V, or VI nuclease, in some cases a Type V nuclease, in preferred embodiments, a Type V-A, V-C, or V-D Cas nuclease, in more preferred embodiments a Type VA nuclease, including but not limited to a Cpf1 nuclease, derivative, or variant: a MAD nuclease, derivative, or variant: a ART nuclease, derivative, or variant: a Csm1 nuclease, derivative, or variant: or an ABW nuclease, derivative, or variant: specific examples are provided herein. In preferred embodiments the nuclease is a Type V-A nuclease. In a preferred embodiments the Type-V-A nuclease is a MAD, ART, or ABW nuclease. In more preferred embodiments Type-V-A nuclease is a MAD nuclease, such as a MAD1, MAD2, MAD3, MAD4, MAD5, MAD6, MAD7, MAD8, MAD9, MAD10, MAD11, MAD12, MAD13, MAD14, MAD15, MAD16, MAD17, MAD18, MAD19, or MAD20 nuclease, preferably a MAD7 nuclease. In other embodiments the nuclease is a ART nuclease, such as an ART1, ART2, ART3, ART4, ART5, ART6, ART7, ART8, ART9, ARTI0, ART11, ART11*, ART12, ART13, ART14, ART15, ART16, ART17, ART18, ART19, ART20, ART21, ART22, ART23, ART24, ART25, ART26, ART27, ART28, ART29, ART30, ART31, ART32, ART33, ART34, or ART35 nuclease, preferably an ART2, ART11, or ART11* nuclease. In certain embodiments the nuclease has an amino acid sequence at least 80, 85, 90, 95, 99, or 100% % identical to the amino acid sequence of MAD2, MAD7, ART2, ART11, or ART11 *. In certain embodiments the the nucleic acid-guided nuclease comprises an amino acid sequence that is at least 80, 85, 90, 95, 99, or 100% identical, preferably at least 90% identical, more preferably at least 95% identical to the amino acid sequence of SEQ ID NO: 37. For any nuclease, the nuclease may include at least one nuclear localization signal (NLS), at least one purification tag, or at least one cleavage site: it will be appreciated that the nuclease may include a purification tag which is removed by cleavage at the cleavage site. In certain embodiments the nuclease includes at least one, two, three, or four NLSs, preferably at least three, more preferably at least four, such as one N-terminal and three C-terminal NLS: this is merely exemplary and it will be appreciated that any combination can be used, e.g., all NLSs at the N-terminus. In preferred embodiments, the nucleic acid-guided nuclease comprises at least five NLS, which can be distributed in any suitable/desired combination of N- and C-terminus: in preferred embodiments, all at the N-terminus. Any suitable NLS or combination of NLSs can be used, in preferred embodiments one or more NLSs comprising any of SEQ ID NOs: 40-56, such as any of SEQ ID NOs: 40, 51, and 56. The guide nucleic acid (gNA), e.g., gRNA, can be any suitable gNA, e.g., gRNA, such as a sgRNA or dual gRNA, as appropriate for the nuclease used. In certain embodiments the gNA, e.g., gRNA, is a dual gNA, e.g., dual gRNA that is not found in natural systems that utilize the particular nuclease, e.g., a Type V-A nuclease, gRNAs can include one or more modified nucleotides, as described herein. The on-target site can be any suitable gene: specific genes can be as described herein. In general the gNA, e.g., gRNA, will comprise (A) a targeter nucleic acid comprising a targeter stem sequence and a spacer sequence; and (B) a modulator nucleic acid comprising a modulator stem sequence complementary to the targeter stem sequence, and, optionally, a 5′ sequence. In certain embodiments the gNA, e.g., gRNA, is an engineered, not naturally occurring gNA, e.g., gRNA. The gNA, e.g., gRNA, can comprise a single polynucleotide. In preferred embodiments the gNA, e.g., gRNA comprises a dual guide nucleic acid, wherein the targeter nucleic acid and the modulator nucleic acid are separate polynucleotides, the dual gNA is capable of binding to and activating a nucleic acid-guided nuclease, that, in a naturally occurring system, is activated by a single crRNA in the absence of a tracrRNA, e.g., a Type V-A nuclease. Any suitable spacer sequence may be used; in certain embodiments the spacer sequence comprises a spacer sequence of any one of SEQ ID NOs: 86-384 and 983-1798. In preferred embodiments, some or all of the gNA comprises RNA, e.g., at least 50%, at least 70%, at least 90%, at least 95%, or 100% of the gNA comprises RNA: in preferred embodiments the gNA is 100% RNA. The gNA, e.g., gRNA, can comprise one or more chemical modifications, such as one or more of a 2′-O-alkyl, a 2′-O-methyl, a phosphorothioate, a phosphonoacetate, a thiophosphonoacetate, a 2′-O-methyl-3′-phosphorothioate, a 2′-O-methyl-3′-phosphonoacetate, a 2′-O-methyl-3′-thiophosphonoacetate, a 2′-deoxy-3′-phosphonoacetate, a 2′-deoxy-3′-thiophosphonoacetate, or a combination thereof. One or more donor templates, e.g., for a mutation in a gene (e.g., a mutation in a PAM, and others as described herein), a transgene to be inserted, a wild-type gene, or other, as described herein, may be used. In certain embodiments, an ssODN that includes the donor template may be used, e.g., a single oligonucleotide comprising appropriate homology arms and the donor template. In other embodiments, two or more ssODNs may be used to provide a complete system for insertion, i.e., homology arms and donor template. In this case, a first ssODN can provide a first homology arm at the 3′ or 5′ end of the donor template, and also include a sequence complementary to a sequence at the 3′ or 5′ end of the donor template so that the two hybridize. In certain embodiments a second ssODN provides a second homology arm at the other end of the donor template, e.g., at the 5′ or 3′ end, and also include a sequence complementary to a sequence at the 5′ or 3′ end of the o the donor template so that the two hybridize. In certain embodiments provided is a kit comprising a composition of this paragraph. In certain embodiments provided is a cell comprising a composition of this paragraph. In certain embodiments provided herein is a method comprising introducing one or more of the compositions of this paragraph into a cell: any suitable method may be used. In a preferred embodiment electroporation is used. An HDR enhancer, e.g., M3814, and/or an anionic polymer, such as non-specific ssODNs or a peptide, e.g., poly-L-glutamic acid (PGA), both of which as described elsewhere herein, may be used. The cell can be any suitable cell, preferably a human cell, more preferably an immune or stem cell, as described below. Also provided is a cell comprising a composition of this paragraph, preferably a human cell, even more preferably a human immune cell or a human stem cell. Exemplary immune cells are a neutrophil, cosinophil, basophil, mast cell, monocyte, macrophage, dendritic cell, natural killer cell, or a lymphocyte: preferably a T cell, more preferably a CAR-T cell. Exemplary stem cells include a human pluripotent, multipotent stem cell, embryonic stem cell, induced pluripotent stem cell, or hematopoietic stem cell: preferably a CD34+ stem cell or an induced pluripotent stem cell (iPSC). In certain embodiments provided herein is a a method of cleaving at or near a target nucleic acid sequence which is at or near an on-target site within a target polynucleotide comprising contacting the target polynucleotide with any of the compositions of this paragraph that include the nucleic acid-guided nuclease complex, wherein the nucleic acid-guided nuclease complex cleaves at least one strand of the target polynucleotide within the on-target site. Also provided herein is a method of editing a genome of a eukaryotic cell comprising delivering any of the compositions of this paragraph that include the nucleic acid-guided nuclease complex into the eukaryotic cell, thereby resulting in editing of the genome of the eukaryotic cell. The composition may be transported into the cell by any suitable method, preferably electroporation. Also provided herein is a method of treating a disease or a disorder comprising administering to a subject in need thereof an effective amount of a composition of this paragraph that includes the nucleic acid-guided nuclease complex or an effective amount of cells modified by treatment with a composition of this paragraph that includes the nucleic acid-guided nuclease complex. Also provided is method of reducing a proportion of mutations in off-target sites in a genome of a cell comprising contacting the cell with a composition of this paragraph that includes the nucleic acid-guided nuclease complex, compared to the proportion if the composition is not used. The reduction can be at least 5, 10, 15, 20, 30, 40, 50, 60, 70, 80, 90, 95, or 99%, preferably at least 20%, more preferably at least 40%, even more preferably at least 60%. The method can also comprise increasing HDR and/or increasing viability and/or expansion capacity of the cells after editing. In certain embodiments provided herein is a method of both increasing HDR at an on-target site in a genome of a cell and decreasing mutations at one or more off-target sites in the genome of the cell comprising the cell with a composition of this paragraph that includes the nucleic acid-guided nuclease complex, thereby both increasing HDR at the on-target site and decreasing the proportion of mutations in off-target sites of the genome of the cell compared to the proportion if the composition is not used. The increase in HDR at the on-target site can be at least 5, 10, 15, 20, 30, 40, 50, 60, 70, 80, 90, 95, or 99%, preferably at least 20%, more preferably at least 40%, even more preferably at least 60%. The decrease in mutations in off-target sites of the genome can be at least 5, 10, 15, 20, 30, 40, 50, 60, 70, 80, 90, 95, or 99%, preferably at least 20%, more preferably at least 40%, even more preferably at least 60%.

In certain embodiments provided herein is a composition comprising (A) a nucleic acid-guided nuclease complex comprising a Type V nuclease and a compatible gNA, e.g., gRNA, wherein the the nucleic acid-guided nuclease complex specifically binds to a target nucleic acid sequence at or near an on-target site and cleaves at or near the target nucleic acid sequence to create a strand break in the on-target site; and (B) a first ssODN. It will be appreciated that a composition may instead provide one or more polynucleotides coding for one or both of the nuclease and the gNA, e.g., gRNA and that cellular machinery is relied on to provide the final nuclease and/or gNA, e.g., gRNA, and such embodiments are included herein wherever compositions and/or methods are described in terms of the nuclease and/or gNA. However, in preferred embodiments the nuclease and the gNA, e.g., gRNA, are provided as is, e.g., either delivered to a cell separately or, more preferably, combined to form a RNP in a form that can be transfected into the cell. Any suitable Type V nuclease complex and ssODN can be used. In certain embodiments, the first ssODN comprises a sequence that is complementary to a sequence flanking the strand break in the on-target site on the 3′ side of the strand break. Additionally or alternatively, the first ssODN can comprise a sequence that is complementary to a sequence flanking the strand break in the on-target site on the 5′ side of the strand break. In certain embodiments, the composition comprises a second ssODN, which can be the same as or different from the first ssODN, comprising a sequence that is complementary to a sequence flanking the strand break in the on-target site on the 5′ side of the strand break and/or on the 3″ side of the strand break. In certain embodiments at least a portion of the first and/or second ssODNs are capable of being integrated at or near the strand break. In certain embodiments the composition further comprises a donor template, which can be incorporated into an ssODN, or can be separate. One or more donor templates, e.g., for a mutation in a gene (e.g., a mutation in a PAM, and others as described herein), a transgene to be inserted, a wild-type gene, or other, as described herein, may be used. In certain embodiments, an ssODN that includes the donor template may be used, e.g., a single oligonucleotide comprising appropriate homology arms and the donor template. In other embodiments, two or more ssODNs may be used to provide a complete system for insertion, i.e., homology arms and donor template. In this case, a first ssODN can provide a first homology arm at the 3′ or 5′ end of the donor template, and also include a sequence complementary to a sequence at the 3′ or 5′ end of the donor template so that the two hybridize. In certain embodiments a second ssODN provides a second homology arm at the other end of the donor template, e.g., at the 5′ or 3′ end, and also include a sequence complementary to a sequence at the 5′ or 3′ end of the o the donor template so that the two hybridize. Generally, the the nucleic acid-guided nuclease complex also binds to one or more off-target nucleic acid sequences at or near one or more off-target sites and cleaves at or near the one or more off-target nucleic acid sequences to create a strand break in the one or more off-target sites. Thus, the composition may further comprise one or more ssODNs that are complementary to a sequence flanking the strand break in the one or more off-target sites, for example a plurality of ssODNs each of which comprises a different sequence complementary to sequences flanking the strand break in the different off-target sites, such as at least 1, 2, 3, 4, 5, 6, 7, 8, 9, 10, 20, 30, 40, 50, 60, 70, 80, 90, 100, 200, 300, 400, 500, 700, or 1000 and/or no more than 2, 3, 4, 5, 6, 7, 8, 9, 10, 20, 30, 40, 50, 60, 70, 80, 90, 100, 200, 300, 400, 500, 700, 1000 or 2000 ssODNs, preferably 10 to 1000 ssODNS, more preferably 100 to 1000 ssODNS, even more preferably 500 to 1000 ssODNs, each of which comprises a different sequence complementary to sequences flanking the strand break in the different off-target sites. In certain embodiments comprising off-target ssODNs, one or more of the ssODNs comprises s a mutation in the PAM, such as a synonymous mutation, as described elsewhere herein. In certain embodiments the Type V nuclease is a Type V-A, V-B, V-C, V-D, or V-E nuclease; in preferred embodiments the nuclease is a Type V-A nuclease. In a preferred embodiments the Type-V-A nuclease is a MAD, ART, or ABW nuclease. In more preferred embodiments Type-V-A nuclease is a MAD nuclease, such as a MAD1, MAD2, MAD3, MAD4, MAD5, MAD6, MAD7, MAD8, MAD9, MAD10, MAD1I, MAD12, MAD13, MAD14, MAD15, MAD16, MAD17, MAD18, MAD19, or MAD20 nuclease, preferably a MAD7 nuclease. In other embodiments the nuclease is a ART nuclease, such as an ART1, ART2, ART3, ART4, ART5, ART6, ART7, ART8, ART9, ART10, ART11, ART11*, ART12, ART13, ART14, ART15, ART16, ART17, ART18, ART19, ART20, ART21, ART22, ART23, ART24, ART25, ART26, ART27, ART28, ART29, ART30, ART31, ART32, ART33, ART34, or ART35 nuclease, preferably an ART2, ART11, or ART11* nuclease. In certain embodiments the nuclease has an amino acid sequence at least 80, 85, 90, 95, 99, or 100% % identical to the amino acid sequence of MAD2, MAD7, ART2, ART11, or ART11*. In certain embodiments the the nucleic acid-guided nuclease comprises an amino acid sequence that is at least 80, 85, 90, 95, 99, or 100% identical, preferably at least 90% identical, more preferably at least 95% identical to the amino acid sequence of SEQ ID NO: 37. For any nuclease, the nuclease may include at least one nuclear localization signal (NLS), at least one purification tag, or at least one cleavage site; it will be appreciated that the nuclease may include a purification tag which is removed by cleavage at the cleavage site. In certain embodiments the nuclease includes at least one, two, three, or four NLSs, preferably at least three, more preferably at least four, such as one N-terminal and three C-terminal NLS: this is merely exemplary and it will be appreciated that any combination can be used, e.g., all NLSs at the N-terminus. In preferred embodiments, the nucleic acid-guided nuclease comprises at least five NLS, which can be distributed in any suitable/desired combination of N- and C-terminus; in preferred embodiments, all at the N-terminus. Any suitable NLS or combination of NLSs can be used, in preferred embodiments one or more NLSs comprising any of SEQ ID NOs: 40-56, such as any of SEQ ID NOs: 40, 51, and 56. In general the gNA, e.g., gRNA, will comprise (A) a targeter nucleic acid comprising a targeter stem sequence and a spacer sequence; and (B) a modulator nucleic acid comprising a modulator stem sequence complementary to the targeter stem sequence, and, optionally, a 5′ sequence. In certain embodiments the gNA, e.g., gRNA, is an engineered, not naturally occurring gNA, e.g., gRNA. The gNA, e.g., gRNA, can comprise a single polynucleotide. In preferred embodiments the gNA, e.g., gRNA comprises a dual guide nucleic acid, wherein the targeter nucleic acid and the modulator nucleic acid are separate polynucleotides, the dual gNA is capable of binding to and activating a nucleic acid-guided nuclease, that, in a naturally occurring system, is activated by a single crRNA in the absence of a tracrRNA, e.g., a Type V-A nuclease. Any suitable spacer sequence may be used: in certain embodiments the spacer sequence comprises a spacer sequence of any one of SEQ ID NOs: 86-384 and 983-1798. In preferred embodiments, some or all of the gNA comprises RNA, e.g., at least 50%, at least 70%, at least 90%, at least 95%, or 100% of the gNA comprises RNA: in preferred embodiments the gNA is 100% RNA. The gNA, e.g., gRNA, can comprise one or more chemical modifications, such as one or more of a 2′-O-alkyl, a 2′-O-methyl, a phosphorothioate, a phosphonoacetate, a thiophosphonoacetate, a 2′-O-methyl-3′-phosphorothioate, a 2′-O-methyl-3′-phosphonoacetate, a 2′-O-methyl-3′-thiophosphonoacetate, a 2′-deoxy-3′-phosphonoacetate, a 2′-deoxy-3′-thiophosphonoacetate, or a combination thereof. The ssODN can be any suitable length, e.g., at least 100, 120, 140, 160, 180, 200, 220, 240, 260, 280, 300, 350, 400, 450, 500, or 1000 and/or not more than 120, 140, 160, 180, 200, 220, 240, 260, 280, 300, 350, 400, 450, 500, 1000, or 2000 nucleotides, for example 100-500 nucleotides. preferably 140-400 nucleotides. Each ssODN may be present in any suitable amount, e.g., at least 50, 60, 70, 80, 90, 100, 150, 200, 250, 300, 350, 400, 450, 500, 550, 600, 700, 800, or 900 and/or not more than 60, 70, 80, 90, 100, 150, 200, 250, 300, 350, 400, 450, 500, 550, 600, 700, 800, 900 or 1000 pmol of each ssODN, for example 50-1000 pmol of each ssODN. In certain embodiments provided herein is a method comprising introducing one or more of the compositions of this paragraph into a cell; any suitable method may be used. In a preferred embodiment electroporation is used. The method can include expaninding and/or differentiating the cell. An HDR enhancer, e.g., M3814, and/or an anionic polymer, such as non-specific ssODNs or a peptide, e.g., poly-L-glutamic acid (PGA), both of which as described elsewhere herein, may be used. The cell can be any suitable cell, preferably a human cell, more preferably an immune or stem cell, as described below. Also provided is a cell comprising a composition of this paragraph, preferably a human cell, even more preferably a human immune cell or a human stem cells. Exemplary immune cells are a neutrophil, eosinophil, basophil, mast cell, monocyte, macrophage, dendritic cell, natural killer cell, or a lymphocyte; preferably a T cell, more preferably a CAR-T cell. Exemplary stem cells include a human pluripotent, multipotent stem cell, embryonic stem cell, induced pluripotent stem cell, or hematopoietic stem cell; preferably a CD34+ stem cell or an induced pluripotent stem cell (iPSC)

In certain embodiments provided herein is a composition comprising a first single-ssODN comprising a sequence complementary to a nucleic acid sequence flanking the double stranded break at the on-target site flanking a double stranded break at an on-target site for a nucleic acid-guided nuclease complex; and a second ssODN comprising a sequence complementary to a nucleic acid sequence flanking a double stranded break at an off-target site (ssODN_{off}) for the nucleic acid-guided nuclease complex. The composition can further comprise the nucleic acid-guided nucleae complex. The composition can comprise, for each integer x representing an off-target site for the nucleic-acid guided nuclease complex, a (ssODN_{off})x wherein each (ssODN_{off})x comprises a sequence complementary to a nucleic acid sequence flanking a double stranded break at an off-target site (x). The number of different integers x can be any suitable number, e.g., at least 2, 3, 4, 5, 6, 7, 8, 9, 10, 20, 30, 40, 50, 60, 70, 80, 90, 100, 120, 140, 160, 180, 200, 250, 300, 350, 400, 450, 500, or 1000 and/or no more than 3, 4, 5, 6, 7, 8, 9, 10, 20, 30, 40, 50, 60, 70, 80, 90, 100, 120, 140, 160, 180, 200, 250, 300, 350, 400, 450, 500, 1000, or 2000, preferably 2-2000, more preferably 100-1000. In certain embodiments the ssODN comprising a sequence complementary to a nucleic acid sequence flanking a double stranded break at an on-target site comprises at least one mutation compared to the wildtype sequence at the on-target site, such as mutation comprising a SNP, an INDEL, and/or a missense mutation. In certain embodiments the ssODN or ssODNs comprising a sequence complementary to a nucleic acid sequence flanking a double stranded break at one or more off-target sites comprises the wildtype sequence for the one or more off-target sites. In certain embodiments the ssODN or ssODNS comprising a sequence complementary to a nucleic acid sequence flanking a double stranded break at one or more off-target sites comprises at least one mutation compared to the wildtype sequence at the one or more off-target sites, such as a synonymous mutation. In certain embodiments the mutation is in the PAM at the one or more off-target sites. In certain embodiments provided herein is a method comprising delivering a composition of this paragraph to a population of cells. The method can further comprise expanding and/or differentiating cells in the population of cells, for example, expanding, or differentiate then expand, or expanding then differentiating, then expanding. The method can produce a population of cells comprising a plurality of genotypes at the on-target site. Delivery can be by any suitable method, preferably electroporation. Nucleotide lengths of the ssODNs can be any suitable length, such as those described herein. Amounts and ratios of ssODNs may be any suitable ratios, also as described herein. In certain embodiments the gRNA is a single gRNA, in other embodiments the gRNA is a dual gRNA. In certain embodiments the gRNA comprises one or more modified nucleotides, as described herein. In certain embodiments, the gRNA targets a specific gene, as described herein. In certain embodiments, the Cas nuclease comprises a Type I, II, III, IV, V, or VI nuclease, in some cases a Type V nuclease, for example, a Type V-A, V-C, or V-D Cas nuclease, such as a Type VA nuclease, including but not limited to a Cpf1 nuclease, derivative, or variant: a MAD nuclease, derivative, or variant: a ART nuclease, derivative, or variant: a Csm1 nuclease, derivative, or variant: or an ABW nuclease, derivative, or variant: specific examples are provided herein.

In certain embodiments provided herein is a composition for integrating at least a portion of a donor template at or near a strand break at an on-target or off-target site in a genome of a cell comprising (A) a donor template lacking one or both homology arms complementary to a sequence or sequences flanking the strand break; and (B) a first ssODN comprising (i) a first portion comprising a sequence complementary to at least a 5′ or 3′ portion of the donor template, and (ii) a second portion comprising a sequence homologous to a sequence flanking the strand break. The composition can further comprise a second ssODN comprising (i) a first portion comprising a sequence complementary to at least a 5′ or 3′ portion of the donor template different from the first ssODN, and (ii) a second portion comprising a sequence homologous to a sequence flanking the strand break. Also provided herein is a method for integrating at least a portion of a donor template at a strand break in a target site in a genome of a cell comprising delivering to the cell a composition of this paragraph, and a nucleic acid guided nuclease complex comprising a nucleic acid-guided nuclease and a compatible gNA, e.g., gRNA, wherein the complex is capable of producing the strand break. The method can further comprise expanding and/or differentiating the cell. Suitable nucleases include any of the nucleases described herein, such as a Type V nuclease, preferably a Type V-A nuclease, such as a Type V-A nuclease as described in previous paragraphs. Suitable gNAs, e.g., gRNAs include any of the gNAs, e.g., gRNAs, described herein, preferably a dual gRNA, in certain embodiment with one or more chemical modifications, also as described herein.

In certain embodiments provided herein is a composition comprising a plurality of ssODNs comprising (A) a first ssODN comprising (i) a first portion comprising a sequence homologous to a sequence upstream of a target site in a genome of a target cell, and (ii) a second portion comprising a sequence comprising at least a portion of a heterologous sequence to be inserted into the genome of the target cell: (B) a second ssODN comprising (i) a first portion comprising a sequence homologous to a sequence downstream of a target site in a genome of a target cell, and (ii) a second portion comprising a sequence at least partially complementary to at least a portion of the heterologous sequence to be inserted into the genome of the target cell; and, optionally, (C) one or more additional ssODNs each comprising (i) a sequence comprising at least a portion of a heterologous sequence to be inserted into the genome of the target cell, and (ii) a second portion comprising a sequence at least partially complementary to at least a portion of the heterologous sequence to be inserted into the genome of the target cell: wherein the plurality of ssODNs comprises the entirety of heterologous sequence to be inserted into the genome of the target cell. The composition can further comprise a nucleic acid-guided nuclease complex comprising a nuclease and a gNA, e.g., gRNA. Suitable nucleases include any of the nucleases described herein, such as a Type V nuclease, preferably a Type V-A nuclease, such as a Type V-A nuclease as described in previous paragraphs. Suitable gNAs, e.g., gRNAs include any of the gNAs, e.g., gRNAs, described herein, preferably a dual gRNA, in certain embodiment with one or more chemical modifications, also as described herein. Also provided herein is a method for inserting a heterologous sequence at or near a target site in a genome of a cell comprising delivering a composition of this paragraph to the cell, where the composition includes a nucleic acid-guided nuclease complex capable of binding to the target site and cleaving at or near the target site. The method may further include expanding and/or differentiating the cell.

In certain embodiment provided herein is a method comprising contacting a population of cells with a composition comprising (A) a nucleic acid-guided nuclease complex comprising a nucleic acid-guided nuclease and a compatible gNA, wherein the complex can bind to and cleave at an on-target site and one or more off-target sites in the genomes of the cells in the population of cells. (B) a ssODN, and (C) one or more ssODNs for one or more of the off-target sites. Suitable nucleases include any of the nucleases described herein, such as a Type V nuclease, preferably a Type V-A nuclease, such as a Type V-A nuclease as described in previous paragraphs. Suitable gNAs, e.g., gRNAs include any of the gNAs, e.g., gRNAs, described herein, preferably a dual gRNA, in certain embodiment with one or more chemical modifications, also as described herein. The method can further comprise expanding and/or differentiating cells in the population of cells; in certain embodiments, at least 10, 20, 30, 40, 50, 60, 70, 80, 90, or 95%, preferably at least 20%, more preferably at least 40%, still more preferably at least 60%, of total genome edits at the target site occur through HDR. In certain embodiments a mutation rate at the one or more off-target sites is at least 10, 20, 30, 40, 50, 60, 70, 80, 90, or 95%, preferably at least 20%, more preferably at least 40%, still more preferably at least 60%, lower that that of the same population of cells treated with the composition lacking the one or more ssODNS for the one or more off-target sites.

In certain embodiment provided herein is a composition comprising (A) a guide RNA (gRNA) comprising (i) a first nucleotide sequence that hybridizes to a target nucleic acid sequence in a genome of a cell, and (ii) a second nucleotide sequence that interacts with a Cas nuclease: (B) the Cas nuclease, comprising an RNA-binding portion that interacts with the second nucleotide sequence of the guide RNA to form a ribonucleoprotein (RNP) complex, wherein the RNP complex (i) specifically binds to the target nucleic acid sequence at an on-target site and cleaves at or near the target nucleic acid sequence to create a double-stranded break in the on-target site, and (ii) also binds to one or more off-target nucleic acid sequences at one or more off-target sites and cleaves at or near the one or more off-target nucleic acid sequences to create a double-strand break in the one or more off-target sites: (C) a first, on-target ssODN comprising a sequence complementary to a sequence flanking the double stranded break in the on-target site, wherein the ssODN integrates into DNA in the on-target site; and (D) a second, off-target ssODN comprising a sequence complementary to a genomic sequence flanking a double stranded break in a first off-target site and integrates into the DNA in the off-target site, wherein the second ssODN comprises (i) homology arms for the off-target site that are more complementary to the genomic sequence at the off-target site than homology arms of the on-target ssODN. In certain embodiments the first ssODN comprises at least one nucleotide modification relative to nucleic acid sequence (native sequence) at the on-target site. In certain embodiments the second ssODN further comprises at least one mutation, e.g., synonymous mutation to reduce or eliminate re-cleavage at the off-target site following integration of the second ssODN, such as a mutation in a PAM sequence of the first off-target site. The composition can further comprise a nucleotide sequence to be inserted at the first off-target site that is identical to a wild-type gene at the first off-target site. The composition can further include a third, fourth, fifth, sixth, seventh, eight, ninth, and/or tenth ssODN, the ssODN(s) being for a second, third, fourth, fifth, sixth, seventh, eight, and/or ninth off-target site. In certain embodiments the gRNA is a dual gRNA. In certain embodiments one or more nucleotides of the gRNA is chemically modified, as described elsewhere herein. The gRNA can include any suitable spacer sequence, such as one of the spacer sequences described herein. In certain embodiments the nuclease is a Type V nuclease, such as a Type V-A, V-C, or V-D nuclease, preferably a Type V-A nuclease, such as Cpf1, MAD, Csm1, ART, or ABW nuclease, or derivative or variant thereof, as described more thoroughly elsewhere herein.

Generally, the homologous region(s) of a ssODN has at least 50% sequence identity to a genomic sequence with which recombination is desired. The homology arms are designed or selected such that they are capable of recombining with the nucleotide sequences flanking the target nucleotide sequence under intracellular conditions. In certain embodiments, where HDR of the non-target strand is desired, the ssODN comprises a first homology arm homologous to a sequence 5′ to the target nucleotide sequence and a second homology arm homologous to a sequence 3′ to the target nucleotide sequence. In certain embodiments, the first homology arm is at least 50% (e.g., at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%) identical to a sequence 5′ to the target nucleotide sequence. In certain embodiments, the second homology arm is at least 50% (e.g., at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%) identical to a sequence 3′ to the target nucleotide sequence. In certain embodiments, when the ssODN sequence and a polynucleotide comprising a target nucleotide sequence are optimally aligned, the nearest nucleotide of the ssODN is within about 1, 5, 10, 15, 20, 25, 50, 75, 100, 200, 300, 400, 500, 1000, 2000, 3000, 4000, or more nucleotides from the target nucleotide sequence.

In certain embodiments, the ssODN further comprises an engineered sequence not homologous to the sequence to be repaired. Such engineered sequence can harbor a barcode and/or a sequence capable of hybridizing with a ssODN-recruiting sequence disclosed herein.

As mentioned previously, in certain embodiments, the ssODN further comprises one or more mutations relative to the genomic sequence, wherein the one or more mutations reduce or prevent cleavage, by the same CRISPR-Cas system, of the ssODN or of a modified genomic sequence with at least a portion of the ssODN sequence incorporated. In certain embodiments, in the ssODN, the PAM adjacent to the target nucleotide sequence and recognized by the Cas nuclease is mutated to a sequence not recognized by the same Cas nuclease. In certain embodiments, in the ssODN, the target nucleotide sequence (e.g., the seed region) is mutated. In certain embodiments, the one or more mutations are silent with respect to the reading frame of a protein-coding sequence encompassing the mutated sites.

The ssODN can be introduced into a cell in linear or circular form. If introduced in linear form, the ends of the ssODN may be protected (e.g., from exonucleolytic degradation) by methods known to those of skill in the art. For example, one or more dideoxynucleotide residues are added to the 3′ terminus of a linear molecule and/or self-complementary oligonucleotides are ligated to one or both ends (see, for example, Chang et al. (1987) PROC. NATL. ACAD SCI USA, 84:4959; Nehls et al. (1996) SCIENCE, 272:886; see also the chemical modifications for increasing stability and/or specificity of RNA disclosed supra). Additional methods for protecting exogenous polynucleotides from degradation include, but are not limited to, addition of terminal amino group(s) and the use of modified internucleotide linkages such as, for example, phosphorothioates, phosphoramidates, and O-methyl ribose or deoxyribose residues. As an alternative to protecting the termini of a linear ssODN, additional lengths of sequence may be included outside of the regions of homology that can be degraded without impacting recombination.

A ssODN can be a component of a vector as described herein, contained in a separate vector, or provided as a separate polynucleotide, such as an oligonucleotide, linear polynucleotide, or synthetic polynucleotide. In certain embodiments, a ssODN is in the same nucleic acid as a sequence encoding the targeter nucleic acid, a sequence encoding the modulator nucleic acid, and/or a sequence encoding the Cas protein, where applicable. In certain embodiments, a ssODN is provided in a separate nucleic acid.

A ssODN can be introduced into a cell as an isolated nucleic acid. Alternatively, a ssODN can be introduced into a cell as part of a vector (e.g., a plasmid) having additional sequences such as, for example, replication origins, promoters and genes encoding antibiotic resistance, that are not intended for insertion into the DNA region of interest. Alternatively, a ssODN can be delivered by viruses (e.g., adenovirus, adeno-associated virus (AAV)). In certain embodiments, the ssODN is introduced as an AAV, e.g., a pseudotyped AAV. The capsid proteins of the AAV can be selected by a person skilled in the art based upon the tropism of the AAV and the target cell type. For example, in certain embodiments, ssODN is introduced into a hepatocyte as AAV8 or AAV9. In certain embodiments, the ssODN is introduced into a hematopoietic stem cell, a hematopoietic progenitor cell, or a T lymphocyte (e.g., CD8+T lymphocyte) as AAV6 or an AAVHSC (see, U.S. Pat. No. 9,890,396). It is understood that the sequence of a capsid protein (VP1, VP2, or VP3) may be modified from a wild-type AAV capsid protein, for example, having at least 50% (e.g., at least 60%, at least 70%, at least 75%, at least 80%, at least 85%, at least 90%, at least 91%, at least 92%, at least 93%, at least 94%, at least 95%, at least 96%, at least 97%, at least 98%, or at least 99%) sequence identity to a wild-type AAV capsid sequence.

The ssODN can be delivered to a cell (e.g., a primary cell) by various delivery methods, such as a viral or non-viral method disclosed herein. In certain embodiments, a non-viral ssODN is introduced into the target cell as a naked nucleic acid or in complex with a liposome or poloxamer. In certain embodiments, a non-viral ssODN is introduced into the target cell by electroporation. In other embodiments, a viral ssODN is introduced into the target cell by infection. The engineered, non-naturally occurring system can be delivered before, after, or simultaneously with the ssODN (see, International (PCT) Application Publication No. WO2017/053729). A skilled person in the art can choose proper timing based upon the form of delivery (consider, for example, the time needed for transcription and translation of RNA and protein components) and the half-life of the molecule(s) in the cell. In particular embodiments, where the modified guide CRISPR-Cas system, e.g., modified dual guide CRISPR-Cas system including the Cas protein is delivered by electroporation (e.g., as an RNP), the ssODN (e.g., as an AAV) is introduced into the cell within 4 hours (e.g., within 1, 2, 3, 4, 5, 6, 7, 8, 9, 10, 11, 12, 13, 14, 15, 16, 17, 18, 19, 20, 25, 30, 35, 40, 45, 50, 55, 60, 90, 120, 150, 180, 210, or 240 minutes) after the introduction of the engineered, non-naturally occurring system.

In certain embodiments, ssODN is conjugated covalently to the modulator nucleic acid. Covalent linkages suitable for this conjugation are known in the art and are described, for example, in U.S. Pat. No. 9,982,278 and Savic et al. (2018) ELIFE 7: e33761. In certain embodiments, the ssODN is covalently linked to the modulator nucleic acid (e.g., the 5′ end of the modulator nucleic acid) through an internucleotide bond. In certain embodiments, the ssODN is covalently linked to the modulator nucleic acid (e.g., the 5′ end of the modulator nucleic acid) through a linker.

In certain embodiments provided herein is a cell comprising any of the compositions of the preceding paragraphs. Accordingly, in another aspect, the present invention provides a cell comprising the non-naturally occurring system or a CRISPR expression system described herein. In certain embodiments, the cell is an immune cell. In certain embodiments, the cell is a T cell. In addition, the present invention provides a cell whose genome has been modified by the modified dual guide CRISPR-Cas system or complex disclosed herein.

The target cells can be mitotic or post-mitotic cells from any organism, such as a bacterial cell, an archaeal cell, a cell of a single-cell eukaryotic organism, a plant cell, an algal cell, e.g., Botryococcus braunii, Chlamydomonas reinhardtii, Nannochloropsis gaditana, Chlorella pyrenoidosa, Sargassum patens C. Agardh, and the like, a fungal cell (e.g., a yeast cell), an animal cell, a cell from an invertebrate animal (e.g. fruit fly, enidarian, cchinoderm, nematode, etc.), a cell from a vertebrate animal (e.g., fish, amphibian, reptile, bird, mammal), a cell from a mammal, a cell from a rodent, or a cell from a human. The types of target cells include but are not limited to a stem cell (e.g., an embryonic stem (ES) cell, an induced pluripotent stem (iPS) cell, a germ cell), a somatic cell (e.g., a fibroblast, a hematopoietic cell, a T lymphocyte (e.g., CD8+ T lymphocyte), an NK cell, a neuron, a muscle cell, a bone cell, a hepatocyte, a pancreatic cell), an in vitro or in vivo embryonic cell of an embryo at any stage (e.g., a 1-cell, 2-cell, 4-cell, 8-cell: stage zebrafish embryo). Cells may be from established cell lines or may be primary cells (i.e., cells and cells cultures that have been derived from a subject and allowed to grow in vitro for a limited number of passages of the culture). For example, primary cultures are cultures that may have been passaged within 0 times, 1 time, 2 times, 4 times, 5 times, 10 times, or 15 times, but not enough times to go through the crisis stage. Typically, the primary cell lines of the present invention are maintained for fewer than 10 passages in vitro. If the cells are primary cells, they may be harvested from an individual by any suitable method. For example, leukocytes may be harvested by apheresis, leukocytapheresis, or density gradient separation, while cells from tissues such as skin, muscle, bone marrow, spleen, liver, pancreas, lung, intestine, or stomach can be harvested by biopsy. The harvested cells may be used immediately, or may be stored under frozen conditions with a cryopreservative and thawed at a later time in a manner as commonly known in the art. In certain embodiments, provided herein is a method of treating a disease or disorder comprising administering to a subject in need thereof an effective amount of a composition as described in the previous paragraph, or an effective amount of cells modified as described in this paragraph. The disease or disorder can be any suitable disorder, such as a disease or disorder described herein.

An engineered, non-naturally occurring system disclosed herein can be delivered into a cell by suitable methods known in the art, including but not limited to ribonucleoprotein (RNP) delivery and “Cas RNA” delivery described below.

In certain embodiments, a guide RNA and a Cas protein can be combined into a RNP complex and then delivered into the cell as a pre-formed complex. This method is suitable for active modification of the genetic or epigenetic information in a cell during a limited time period. For example, where the Cas protein has nuclease activity to modify the genomic DNA of the cell, the nuclease activity only needs to be retained for a period of time to allow DNA cleavage, and prolonged nuclease activity may increase off-targeting. Similarly, certain epigenetic modifications can be maintained in a cell once established and can be inherited by daughter cells.

A “ribonucleoprotein” or “RNP,” as used herein, includes a complex comprising a nucleoprotein and a ribonucleic acid. A “nucleoprotein” as used herein includes a protein capable of binding a nucleic acid (e.g., RNA, DNA). Where the nucleoprotein binds a ribonucleic acid it is referred to as “ribonucleoprotein.” The interaction between the ribonucleoprotein and the ribonucleic acid may be direct, e.g., by covalent bond, or indirect, e.g., by non-covalent bond (e.g. electrostatic interactions (e.g. ionic bond, hydrogen bond, halogen bond), van der Waals interactions (e.g. dipole-dipole, dipole-induced dipole, London dispersion), ring stacking (pi effects), hydrophobic interactions, and the like). In certain embodiments, the ribonucleoprotein includes an RNA-binding motif non-covalently bound to the ribonucleic acid. For example, positively charged aromatic amino acid residues (e.g., lysine residues) in the RNA-binding motif may form electrostatic interactions with the negative nucleic acid phosphate backbones of the RNA.

To ensure efficient loading of the Cas protein, the targeter nucleic acid and the modulator nucleic acid can be provided in excess molar amount (e.g., about 2 fold, about 3 fold, about 4 fold, or about 5 fold) relative to the Cas protein. In certain embodiments, the targeter nucleic acid and the modulator nucleic acid are annealed under suitable conditions prior to complexing with the Cas protein. In other embodiments, the targeter nucleic acid, the modulator nucleic acid, and the Cas protein are directly mixed together to form an RNP.

A variety of delivery methods can be used to introduce an RNP disclosed herein into a cell. Exemplary delivery methods or vehicles include but are not limited to microinjection, liposomes (see, e.g., U.S. Patent Publication No. 2017/0107539) such as molecular trojan horses liposomes that delivers molecules across the blood brain barrier (see, Pardridge et al. (2010) COLD SPRING HARB. PROTOC., doi: 10.1101/pdb.prot5407), immunoliposomes, virosomes, microvesicles (e.g., exosomes and ARMMs), polycations, lipid: nucleic acid conjugates, electroporation, cell permeable peptides (see, U.S. Patent Publication No. 2018/0363009), nanoparticles, nanowires (see, Shalek et al. (2012) NANO LETTERS, 12:6498), exosomes, and perturbation of cell membrane (e.g., by passing cells through a constriction in a microfluidic system, see, U.S. Patent Publication No. 2018/0003696). In certain embodiments the delivery method is electroporation. Where the target cell is a proliferating cell, the efficiency of RNP delivery can be enhanced by cell cycle synchronization (see, U.S. Patent Publication No. 2018/0044700).

In other embodiments, a system is delivered into a cell in a “Cas RNA” approach, i.e., delivering a targeter nucleic acid, a modulator nucleic acid, and an RNA (e.g., messenger RNA (mRNA)) encoding a Cas protein. The RNA encoding the Cas protein can be translated in the cell and form a complex with the targeter nucleic acid and the modulator nucleic acid intracellularly. Similar to the RNP approach, RNAs have limited half-lives in cells, even though stability-increasing modification(s) can be made in one or more of the RNAs. Accordingly, the “Cas RNA” approach is suitable for active modification of the genetic or epigenetic information in a cell during a limited time period, such as DNA cleavage, and has the advantage of reducing off-targeting.

The mRNA can be produced by transcription of a DNA comprising a regulatory element operably linked to a Cas coding sequence. Given that multiple copies of Cas protein can be generated from one mRNA, the targeter nucleic acid and the modulator nucleic acid are generally provided in excess molar amount (e.g., at least 5 fold, at least 10 fold, at least 20 fold, at least 30 fold, at least 50 fold, or at least 100 fold) relative to the mRNA. In certain embodiments, the targeter nucleic acid and the modulator nucleic acid are annealed under suitable conditions prior to delivery into the cells. In other embodiments, the targeter nucleic acid and the modulator nucleic acid are delivered into the cells without annealing in vitro. In certain embodiments, a modified dual guide nucleic acid system is used. In certain embodiments, a modified single guide nucleic acid system is used.

A variety of delivery systems can be used to introduce an “Cas RNA” system into a cell. Non-limiting examples of delivery methods or vehicles include microinjection, biolistic particles, liposomes (see, e.g., U.S. Patent Publication No. 2017/0107539) such as molecular trojan horses liposomes that delivers molecules across the blood brain barrier (see, Pardridge et al. (2010) COLD SPRING HARB. PROTOC., doi:10.1101/pdb.prot5407), immunoliposomes, virosomes, polycations, lipid: nucleic acid conjugates, electroporation, nanoparticles, nanowires (see, Shalek et al. (2012) NANO LETTERS, 12:6498), exosomes, and perturbation of cell membrane (e.g., by passing cells through a constriction in a microfluidic system, see, U.S. Patent Publication No. 2018/0003696). Specific examples of the “nucleic acid only” approach by electroporation are described in International (PCT) Publication No. WO2016/164356.

In other embodiments, a composition is delivered into a cell in the form of a targeter nucleic acid, a modulator nucleic acid, and a DNA comprising a regulatory element operably linked to a Cas coding sequence. The DNA can be provided in a plasmid, viral vector, or any other form described in the “CRISPR Expression Systems” subsection. Such delivery method may result in constitutive expression of Cas protein in the target cell (e.g., if the DNA is maintained in the cell in an episomal vector or is integrated into the genome), and may increase the risk of off-targeting which is undesirable when the Cas protein has nuclease activity. Notwithstanding, this approach is useful when the Cas protein comprises a non-nuclease effector (e.g., a transcriptional activator or repressor). It is also useful for research purposes and for genome editing of plants.

In certain embodiments provided herein is a method of cleaving a target DNA having a target nucleotide sequence, the method comprising contacting the target DNA with a composition comprising (i) a guide RNA (gRNA) comprising (a) a first nucleotide sequence that hybridizes to the target DNA sequence in the genome of a cell, and (b) a second nucleotide sequence that interacts with a Cas nuclease; (ii) the Cas nuclease, comprising an RNA-binding portion that interacts with the second nucleotide sequence of the guide RNA to form a ribonucleoprotein (RNP) complex, wherein the RNP complex (a) specifically binds and cleaves the target DNA sequence to create a double-stranded break at an on-target site, and (b) potentially also binds and cleaves one or more non-target DNA sequences at one or more off-target sites; (iii) a ssODN comprising a sequence complementary to a nucleic acid sequence flanking a double stranded break at an on-target site for a nucleic acid-guided nuclease complex; and (iv) a ssODN comprising a sequence complementary to a nucleic acid sequence flanking a double stranded break at an off-target site (ssODN_{off}) for the nucleic acid-guided nuclease complex, wherein the second ssODN comprises (a) homology arms for the off-target site that are more complementary to the genomic sequence at the off-target site than homology arms of the on-target ssODN: thereby resulting in cleavage of the target DNA. The first ssODN can comprise at least one nucleotide modification relative to the target DNA sequence. The second ssODN can further comprise at least one synonymous mutation to prevent re-cleavage of the non-target DNA following incorporation of the second ssODN into the genome of the cell, such as a mutation in a PAM sequence of the first off-target site. The second ssODN can comprise a nucleotide sequence to be inserted at the off-target site that is identical to the wild-type gene at the first off-target site. The composition may further comprise additional off-target ssODNs, different from the second ssODN, for example, at least a third, fourth, fifth, sixth, seventh, eighth, ninth, tenth, ssODN, each of which targets a different off-target site and each of which has homology arms that are more specific to the genomic sequence at its particular off-target site than homology arms of the on-target ssODN. Further embodiments include even more off-target ssODNs, for example, at least 10, 12, 15, 17, 20, 25, 30, 35, 40, 45, 50, 60, 70, 80, 90, 100, 120, 150, or 200 different off-target ssODNs and/or not more than 12, 15, 17, 20, 25, 30, 35, 40, 45, 50, 60, 70, 80, 90, 100, 120, 150, 200 or 500 different off-target ssODNs. Nucleotide lengths of the ssODNs can be any suitable length, such as those described herein. Ratios of ssODNs may be any suitable ratios, also as described herein. In certain embodiments the gRNA is a single gRNA, in other embodiments the gRNA is a dual gRNA. In certain embodiments the gRNA comprises one or more modified nucleotides, as described herein. In certain embodiments, the gRNA targets a specific gene, as described herein. In certain embodiments, the Cas nuclease comprises a Type I, II, III, IV, V, or VI nuclease, in some cases a Type V nuclease, for example, a Type V-A, V-C, or V-D Cas nuclease, such as a Type VA nuclease, including but not limited to a Cpf1 nuclease, derivative, or variant; a MAD nuclease, derivative, or variant: a ART nuclease, derivative, or variant; a Csm1 nuclease, derivative, or variant; or an ABW nuclease, derivative, or variant; specific examples are provided herein.

In certain embodiments, provided herein is a method of reducing the proportion of mutations in off-target sites compared to an on-target site comprising a target DNA sequence in a genome of a cell comprising contacting the cell with a composition comprising (i) a guide RNA (gRNA) comprising (a) a first nucleotide sequence that hybridizes to the target DNA sequence, and (b) a second nucleotide sequence that interacts with a Cas nuclease: (ii) the Cas nuclease, comprising an RNA-binding portion that interacts with the second nucleotide sequence of the guide RNA to form a ribonucleoprotein (RNP) complex, wherein the RNP complex (a) specifically binds and cleaves the target DNA sequence to create a double-stranded break at the on-target site, and (b) potentially also binds and cleaves one or more non-target DNA sequences at one or more off-target sites; (iii) a first single-stranded DNA oligonucleotide (ssODN) that is complementary to and hybridizes with a genomic sequence flanking the double stranded break at the on-target site and integrates into DNA at the on-target site; and (iv) a second ssODN that comprises a sequence that is complementary to and hybridizes with a genomic sequence flanking a double stranded break, if present, at a first off-target site and integrates into the DNA at the off-target site, wherein the second ssODN comprises (a) homology arms for the off-target site that are more complementary to the genomic sequence at the off-target site than homology arms of the on-target ssODN: thereby reducing the proportion of mutations in off-target sites of the genome of the cell compared to the proportion if the composition is not used, e.g., if a composition comprising the on-target materials but not the off-target materials is used. The first ssODN can comprise at least one nucleotide modification relative to the target DNA sequence. The second ssODN can further comprise at least one synonymous mutation to prevent re-cleavage of the non-target DNA following incorporation of the second ssODN into the genome of the cell, such as a mutation in a PAM sequence of the first off-target site. The second ssODN can comprise a nucleotide sequence to be inserted at the off-target site that is identical to the wild-type gene at the first off-target site. The composition may further comprise additional off-target ssODNs, different from the second ssODN, for example, at least a third, fourth, fifth, sixth, seventh, eighth, ninth, tenth, ssODN, each of which targets a different off-target site and each of which has homology arms that are more specific to the genomic sequence at its particular off-target site than homology arms of the on-target ssODN. Further embodiments include even more off-target ssODNs, for example, at least 10, 12, 15, 17, 20, 25, 30, 35, 40, 45, 50, 60, 70, 80, 90, 100, 120, 150, or 200 different off-target ssODNs and/or not more than 12, 15, 17, 20, 25, 30, 35, 40, 45, 50, 60, 70, 80, 90, 100, 120, 150, 200 or 500 different off-target ssODNs. Nucleotide lengths of the ssODNs can be any suitable length, such as those described herein. Ratios of ssODNs may be any suitable ratios, also as described herein. In certain embodiments the gRNA is a single gRNA, in other embodiments the gRNA is a dual gRNA. In certain embodiments the gRNA comprises one or more modified nucleotides, as described herein. In certain embodiments, the gRNA targets a specific gene, as described herein. In certain embodiments, the Cas nuclease comprises a Type I, II, III, IV, V, or VI nuclease, in some cases a Type V nuclease, for example, a Type V-A, V-C, or V-D Cas nuclease, such as a Type VA nuclease, including but not limited to a Cpf1 nuclease, derivative, or variant; a MAD nuclease, derivative, or variant; a ART nuclease, derivative, or variant; a Csm1 nuclease, derivative, or variant; or an ABW nuclease, derivative, or variant; specific examples are provided herein.

It will be appreciated that the methods and compositions provided herein increase the proportion of HDR compared to NHEJ in a genome, while also decreasing the amount of off-target mutations. In addition, or alternatively, the methods and compositions provided herein can also increase the viability/expansion capacity of cells after editing. Thus, provided herein is a method of both increasing HDR at an on-target site in a genome of a cell and decreasing mutations at one or more off-target sites in the genome of the cell comprising contacting the cell with a composition comprising (i) a guide RNA (gRNA) comprising (a) a first nucleotide sequence that hybridizes to the target DNA sequence, and (b) a second nucleotide sequence that interacts with a Cas nuclease; (ii) the Cas nuclease, comprising an RNA-binding portion that interacts with the second nucleotide sequence of the guide RNA to form a ribonucleoprotein (RNP) complex, wherein the RNP complex (a) specifically binds and cleaves the target DNA sequence to create a double-stranded break at the on-target site, and (b) potentially also binds and cleaves one or more non-target DNA sequences at one or more off-target sites; (iii) a first single-stranded DNA oligonucleotide (ssODN) that is complementary to and hybridizes with a genomic sequence flanking the double stranded break at the on-target site and integrates into DNA at the on-target site; and (iv) a second ssODN that comprises a sequence that is complementary to and hybridizes with a genomic sequence flanking a double stranded break, if present, at a first off-target site and integrates into the DNA at the off-target site, wherein the second ssODN comprises (a) homology arms for the off-target site that are more complementary to the genomic sequence at the off-target site than homology arms of the on-target ssODN; thereby both increasing HDR at the on-target site and decreasing the proportion of mutations in the off-target site of the genome of the cell compared to if the composition is not used, e.g., if a composition comprising the on-target materials but not the off-target materials is used. The first ssODN can comprise at least one nucleotide modification relative to the target DNA sequence. The second ssODN can further comprise at least one synonymous mutation to prevent re-cleavage of the non-target DNA following incorporation of the second ssODN into the genome of the cell, such as a mutation in a PAM sequence of the first off-target site. The second ssODN can comprise a nucleotide sequence to be inserted at the off-target site that is identical to the wild-type gene at the first off-target site. The composition may further comprise additional off-target ssODNs, different from the second ssODN, for example, at least a third, fourth, fifth, sixth, seventh, eighth, ninth, tenth, ssODN, each of which targets a different off-target site and each of which has homology arms that are more specific to the genomic sequence at its particular off-target site than homology arms of the on-target ssODN. Further embodiments include even more off-target ssODNs, for example, at least 10, 12, 15, 17, 20, 25, 30, 35, 40, 45, 50, 60, 70, 80, 90, 100, 120, 150, or 200 different off-target ssODNs and/or not more than 12, 15, 17, 20, 25, 30, 35, 40, 45, 50, 60, 70, 80, 90, 100, 120, 150, 200 or 500 different off-target ssODNs. Nucleotide lengths of the ssODNs can be any suitable length, such as those described herein. Ratios of ssODNs may be any suitable ratios, also as described herein. In certain embodiments the gRNA is a single gRNA, in other embodiments the gRNA is a dual gRNA. In certain embodiments the gRNA comprises one or more modified nucleotides, as described herein. In certain embodiments, the gRNA targets a specific gene, as described herein. In certain embodiments, the Cas nuclease comprises a Type I, II, III, IV, V, or VI nuclease, in some cases a Type V nuclease, for example, a Type V-A. V-C, or V-D Cas nuclease, such as a Type VA nuclease, including but not limited to a Cpf1 nuclease, derivative, or variant; a MAD nuclease, derivative, or variant; a ART nuclease, derivative, or variant; a Csm1 nuclease, derivative, or variant; or an ABW nuclease, derivative, or variant; specific examples are provided herein.

In certain embodiments, the engineered, non-naturally occurring system has high efficiency. For example, in certain embodiments, at least 10%, at least 20%, at least 30%, at least 40%, at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, or at least 99% of a population of nucleic acids having the target nucleotide sequence and a cognate PAM, when contacted with the engineered, non-naturally occurring system, is targeted, cleaved, or modified. In certain embodiments, the genomes of at least 10%, at least 20%, at least 30%, at least 40%, at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, or at least 99% of a population of cells, when contacted with the engineered, non-naturally occurring system, are targeted, cleaved, or modified.

It has been observed that the occurrence of on-target events and the occurrence of off-target events are generally correlated. For certain therapeutic purposes, low on-target efficiency can be tolerated and low off-target frequency is more desirable. For example, when editing or modifying a proliferating cell that will be delivered to a subject and proliferate in vivo, tolerance to off-target events is low. Prior to delivery, however, it is possible to assess the on-target and off-target events, thereby selecting one or more colonies that have the desired edit or modification and lack any undesired edit or modification.

The methods disclosed herein can be suitable for such use. In certain embodiments, when a population of nucleic acids having the target nucleotide sequence and a cognate PAM is contacted with one of the engineered, non-naturally occurring systems disclosed herein, the frequency of off-target events (e.g., targeting, cleavage, or modification, depending on the function of the CRISPR-Cas system) is reduced by at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, or at least 99% relative to the frequency of off-target events when using the corresponding CRISPR system not containing off-target ssODNs under the same conditions. In certain embodiments, when genomic DNA having the target nucleotide sequence and a cognate PAM is contacted with one of the engineered, non-naturally occurring systems disclosed herein in a population of cells, the frequency of off-target events (e.g., targeting, cleavage, or modification, depending on the function of the CRISPR-Cas system) is reduced by at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, or at least 99% % relative to the frequency of off-target events when using the corresponding CRISPR system not containing off-target ssODN under the same conditions. In certain embodiments, when delivered into a population of cells comprising genomic DNA having the target nucleotide sequence and a cognate PAM, the frequency of off-target events (e.g., targeting, cleavage, or modification, depending on the function of the CRISPR-Cas system) in the cells receiving one of the engineered, non-naturally occurring systems disclosed herein is reduced by at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, or at least 99% % relative to the frequency of off-target events when using the corresponding CRISPR system not containing off-target ssODN under the same conditions. Methods of assessing off-target events were summarized in Lazzarotto et al. (2018) NAT PROTOC. 13 (11): 2615-42, and include discovery of in situ Cas off-targets and verification by sequencing (DISCOVER-seq) as disclosed in Wienert et al. (2019) SCIENCE 364 (6437): 286-89: genome-wide unbiased identification of double-stranded breaks (DSBs) enabled by sequencing (GUIDE-seq) as disclosed in Kleinstiver et al. (2016) NAT. BIOTECH. 34:869-74; circularization for in vitro reporting of cleavage effects by sequencing (CIRCLE-seq) as described in Kocak et al. (2019) NAT. BIOTECH. 37:657-66. In certain embodiments, the off-target events include targeting, cleavage, or modification at a given off-target locus (e.g., the locus with the highest occurrence of off-target events detected). In certain embodiments, the off-target events include targeting, cleavage, or modification at all the loci with detectable off-target events, collectively.

A composition comprising ssODNs as described herein, may be delivered to a cell by introducing a pre-formed ribonucleoprotein (RNP) complex into the cell. Alternatively, one or more components may be expressed in the cell: it will be appreciated that segments containing modified nucleotides should be introduced into the cells, but unmodified segments can be expressed in the cell. Exemplary methods of delivery are known in the art and described in, for example, U.S. Pat. Nos. 10,113,167 and 8,697,359 and U.S. Patent Application Publication Nos. 2015/0344912, 2018/0044700, 2018/0003696, 2018/0119140, 2017/0107539, 2018/0282763, and 2018/0363009.

It is understood that contacting a DNA (e.g., genomic DNA) in a cell with a composition comprising an ssODN as described herein does not require delivery of all components of the complex into the cell. For examples, one or more of the components may be pre-existing in the cell. In certain embodiments, the cell (or a parental/ancestral cell thereof) has been engineered to express the Cas protein, and the targeter nucleic acid (or a nucleic acid comprising a regulatory element operably linked to a nucleotide sequence encoding the targeter nucleic acid) and the modulator nucleic acid (or a nucleic acid comprising a regulatory element operably linked to a nucleotide sequence encoding the modulator nucleic acid) are delivered into the cell, in some cases, where one or the other, or both, contains one or more modified nucleotides at the 3′ and/or 5′ ends. In certain embodiments, the cell (or a parental/ancestral cell thereof) has been engineered to express the modulator nucleic acid, and the Cas protein (or a nucleic acid comprising a regulatory element operably linked to a nucleotide sequence encoding the Cas protein) and the targeter nucleic acid (or a nucleic acid comprising a regulatory element operably linked to a nucleotide sequence encoding the targeter nucleic acid) are delivered into the cell, in some cases where the targeter nucleic acid contains one or more modified nucleotides at the 3′ and/or 5′ ends. In certain embodiments, the cell (or a parental/ancestral cell thereof) has been engineered to express the Cas protein and the targeter nucleic acid, and the modulator nucleic acid (or a nucleic acid comprising a regulatory element operably linked to a nucleotide sequence encoding the modulator nucleic acid) is delivered into the cell, in some cases, where the modulator nucleic acid contains one or more modified nucleotides at the 3′ and/or 5′ ends.

In certain embodiments, the target DNA is in the genome of a target.

### II. High Efficiency Transgene Insertion

Recent advances have been made in precise genome targeting technologies. For example, specific loci in genomic DNA can be targeted, edited, or otherwise modified by designer meganucleases, zinc finger nucleases, or transcription activator-like effectors (TALEs). Furthermore, the CRISPR-Cas systems of bacterial and archaeal adaptive immunity have been adapted for precise targeting of genomic DNA in eukaryotic cells. Compared to the earlier generations of genome editing tools, the CRISPR-Cas systems are easy to set up, scalable, and amenable to targeting multiple positions within the eukaryotic genome, thereby providing a major resource for new applications in genome engineering. In certain embodiments, provided herein are compositions, methods, and/or kits for genome engineering. In certain embodiments, provided herein are compositions, methods, and/or kits for genome engineering of eukaryotic cells. In certain embodiments, provided herein are compositions, methods, and/or kits for genome engineering of human cells. In certain embodiments, provided herein are compositions, methods, and/or kits for genome engineering of human immune or stem cells. In certain embodiments, provided herein are compositions, methods, and/or kits for efficient genome engineering. In certain embodiments, provided herein are compositions, methods, and/or kits for efficient genome engineering via optimized compositions and/or methods. In certain embodiments, provided herein are compositions, methods, and/or kits comprising nucleases. In certain embodiments, provided herein are compositions, methods, and/or kits comprising nucleic acid-guided nucleases, e.g., CRISPR-cas nucleases. In certain embodiments, provided herein are compositions, methods, and/or kits comprising guide nucleic acids (gNAs). In certain embodiments, provided herein are compositions, methods, and/or kits comprising molecules that improve the efficiency of genome editing. In certain embodiments, provided herein are compositions, methods, and/or kits comprising molecules that stabilize RNPs, e.g., RNP stabilizer. In certain embodiments, provided herein are compositions, methods, and/or kits comprising molecules that inhibit non-homologous end joining (NHEJ), e.g., NHEJ inhibitor. In certain embodiments, provided herein are compositions, methods, and/or kits comprising improved combinations and/or concentrations of one or more of the following items: (1) one or more guide nucleic acids (gNA), (2) one or more nucleases, (3) one or more donor templates, (4) one or more RNP stabilizers, (5) one or more NHEJ inhibitors, (6) one or more cell growth and/or recovery mediums, and/or (7) one or more human target cells.

In certain embodiments, provided herein are compositions, methods, and/or kits comprising at least one of the seven items. In certain embodiments, provided herein are compositions, methods, and/or kits comprising at least two of the seven items. In certain embodiments, provided herein are compositions, methods, and/or kits comprising at least three of the seven items. In certain embodiments, provided herein are compositions, methods, and/or kits comprising at least four of the seven items. In certain embodiments, provided herein are compositions, methods, and/or kits comprising at least five of the seven items. In certain embodiments, provided herein are compositions, methods, and/or kits comprising at least six of the seven items. In certain embodiments, provided herein are compositions, methods, and/or kits comprising all seven items.

In certain embodiments, provided herein are compositions, methods, and/or kits comprising one or more nucleic acid guided nucleases, i.e., nuclease. In certain embodiments, provided herein are compositions, methods, and/or kits comprising one or more nucleases that further comprise at least one of the six additional items. In certain embodiments, provided herein are compositions, methods, and/or kits comprising one or more nucleases that further comprise at least two of the six additional items. In certain embodiments, provided herein are compositions, methods, and/or kits comprising one or more nucleases that further comprise at least three of the six additional items. In certain embodiments, provided herein are compositions, methods, and/or kits comprising one or more nucleases that further comprise at least four of the six additional items. In certain embodiments, provided herein are compositions, methods, and/or kits comprising one or more nucleases that further comprise at least five of the six additional items. In certain embodiments, provided herein are compositions, methods, and/or kits comprising one or more nucleases that further comprise all six additional items.

In certain embodiments, provided herein are compositions, methods, and/or kits comprising one or more guide nucleic acids. In certain embodiments, provided herein are compositions, methods, and/or kits comprising one or more guide nucleic acids that further comprise at least one of the six additional items. In certain embodiments, provided herein are compositions, methods, and/or kits comprising one or more guide nucleic acids that further comprise at least two of the six additional items. In certain embodiments, provided herein are compositions, methods, and/or kits comprising one or more guide nucleic acids that further comprise at least three of the six additional items. In certain embodiments, provided herein are compositions, methods, and/or kits comprising one or more guide nucleic acids that further comprise at least four of the six additional items. In certain embodiments, provided herein are compositions, methods, and/or kits comprising one or more guide nucleic acids that further comprise at least five of the six additional items. In certain embodiments, provided herein are compositions, methods, and/or kits comprising one or more guide nucleic acids that further comprise all six additional items.

In certain embodiments, provided herein are compositions, methods, and/or kits comprising one or more guide nucleic acids and one or more nucleases. In certain embodiments, provided herein are compositions, methods, and/or kits comprising one or more guide nucleic acids and one or more nucleases that further comprise at least one of the five additional items. In certain embodiments, provided herein are compositions, methods, and/or kits comprising one or more guide nucleic acids and one or more nucleases that further comprise at least two of the five additional items. In certain embodiments, provided herein are compositions, methods, and/or kits comprising one or more guide nucleic acids and one or more nucleases that further comprise at least three of the five additional items. In certain embodiments, provided herein are compositions, methods, and/or kits comprising one or more guide nucleic acids and one or more nucleases that further comprise at least four of the five additional items. In certain embodiments, provided herein are compositions, methods, and/or kits comprising one or more guide nucleic acids and one or more nucleases that further comprise all five additional items.

In certain embodiments, provided herein are compositions, methods, and/or kits comprising one or more guide nucleic acids and one or more RNP stabilizers. In certain embodiments, provided herein are compositions, methods, and/or kits comprising one or more guide nucleic acids and one or more RNP stabilizers that further comprise at least one of the five additional items. In certain embodiments, provided herein are compositions, methods, and/or kits comprising one or more guide nucleic acids and one or more RNP stabilizers that further comprise at least two of the five additional items. In certain embodiments, provided herein are compositions, methods, and/or kits comprising one or more guide nucleic acids and one or more RNP stabilizers that further comprise at least three of the five additional items. In certain embodiments, provided herein are compositions, methods, and/or kits comprising one or more guide nucleic acids and one or more RNP stabilizers that further comprise at least four of the five additional items. In certain embodiments, provided herein are compositions, methods, and/or kits comprising one or more guide nucleic acids and one or more RNP stabilizers that further comprise all five additional items.

In certain embodiments, provided herein are compositions, methods, and/or kits comprising one or more guide nucleic acids, one or more RNP stabilizers, and one or more nucleases. In certain embodiments, provided herein are compositions, methods, and/or kits comprising one or more guide nucleic acids, one or more RNP stabilizers, and one or more nucleases that further comprise at least one of the four additional items. In certain embodiments, provided herein are compositions, methods, and/or kits comprising one or more guide nucleic acids, one or more RNP stabilizers, and one or more nucleases that further comprise at least two of the four additional items. In certain embodiments, provided herein are compositions, methods, and/or kits comprising one or more guide nucleic acids, one or more RNP stabilizers, and one or more nucleases that further comprise at least three of the four additional items. In certain embodiments, provided herein are compositions, methods, and/or kits comprising one or more guide nucleic acids, one or more RNP stabilizers, and one or more nucleases that further comprise all four additional items.

In certain embodiments, provided herein are compositions, methods, and/or kits comprising one or more human target cells. In certain embodiments, provided herein are compositions, methods, and/or kits comprising one or more human target cells that further comprise at least one of the six additional items. In certain embodiments, provided herein are compositions, methods, and/or kits comprising one or more human target cells that further comprise at least two of the six additional items. In certain embodiments, provided herein are compositions, methods, and/or kits comprising one or more human target cells that further comprise at least three of the six additional items. In certain embodiments, provided herein are compositions, methods, and/or kits comprising one or more human target cells that further comprise at least four of the six additional items. In certain embodiments, provided herein are compositions, methods, and/or kits comprising one or more human target cells that further comprise at least five of the six additional items. In certain embodiments, provided herein are compositions, methods, and/or kits comprising one or more human target cells that further comprise all six additional items.

In certain embodiments, provided herein are compositions, methods, and/or kits comprising one or more human target cells and one or more NHEJ inhibitor. In certain embodiments, provided herein are compositions, methods, and/or kits comprising one or more human target cells and one or more NHEJ inhibitor that further comprise at least one of the five additional items. In certain embodiments, provided herein are compositions, methods, and/or kits comprising one or more human target cells and one or more NHEJ inhibitor that further comprise at least two of the five additional items. In certain embodiments, provided herein are compositions, methods, and/or kits comprising one or more human target cells and one or more NHEJ inhibitor that further comprise at least three of the five additional items. In certain embodiments, provided herein are compositions, methods, and/or kits comprising one or more human target cells and one or more NHEJ inhibitor that further comprise at least four of the five additional items. In certain embodiments, provided herein are compositions, methods, and/or kits comprising one or more human target cells and one or more NHEJ inhibitor that further comprise all five additional items.

In certain embodiments, provided herein are compositions, methods, and/or kits comprising one or more guide nucleic acids, one or more nucleases, and one or more human target cells. In certain embodiments, provided herein are compositions, methods, and/or kits comprising one or more guide nucleic acids, one or more nucleases, and one or more human target cells that further comprise at least one of the four additional items. In certain embodiments, provided herein are compositions, methods, and/or kits comprising one or more guide nucleic acids, one or more nucleases, and one or more human target cells that further comprise at least two of the four additional items. In certain embodiments, provided herein are compositions, methods, and/or kits comprising one or more guide nucleic acids, one or more nucleases, and one or more human target cells that further comprise at least three of the four additional items. In certain embodiments, provided herein are compositions, methods, and/or kits comprising one or more guide nucleic acids, one or more nucleases, and one or more human target cells that further comprise all four additional items. In certain embodiments comprising one or more nucleases, and one or more human target cells, the compositions, methods, and/or kits further can comprise one or more RNP stabilizers, one or more donor templates, and/or one or more NHEJ inhibitors

In certain embodiments, provide herein are compositions, methods, and/or kits wherein the optimized combinations and/or concentrations, e.g., condition and/or treatment, of gNA, nuclease, donor template, RNP stabilizers, and/or NHEJ inhibitors result in at least 1.1, 1.15, 1.2, 1.25, 1.3, 1.35, 1.4, 1.45, 1.5, 1.55, 1.6, 1.65, 1.7, 1.75, 1.8, 1.85, 1.9, 1.95, 2, 2.25, 2.5, 2.75, 3, 4, 5, 6, 7, 8, or 9-fold and/or not more than 1.15, 1.2, 1.25, 1.3, 1.35, 1.4, 1.45, 1.5, 1.55, 1.6, 1.65, 1.7, 1.75, 1.8, 1.85, 1.9, 1.95, 2, 2.25, 2.5, 2.75, 3, 4, 5, 6, 7, 8, 9, or 10-fold increased editing via homology directed repair (HDR) as compared to editing via NHEJ, for example 1.1-10-fold increased editing, preferably 1.1-5-fold increased editing, even more preferably 1.1-3-fold increased editing, yet more preferably 1.1-2-fold increased editing.

In certain embodiments, provided herein are compositions, methods, and/or kits comprising one or more additives that stabilize RNPs, e.g., RNP stabilizer. In certain embodiments, the one or more additives that stabilize RNPs are combined with the nuclease and the guide nucleic acid. In certain embodiments, the one or more additives that stabilize RNPs are combined with the guide nucleic acid prior to combination with the nuclease. In certain embodiments, the one or more additives that stabilize RNPs are combined with the nuclease prior to combination with the guide nucleic acid. In certain embodiments, the one or more additives that stabilize RNPs are combined with the pre-formed RNP complex comprising one or more nucleases and a guide nucleic acid. In certain embodiments, the one or more additives that stabilize RNPs prevent aggregation and/or support dispersion of RNP complexes in a population of RNPs.

In certain embodiments, an RNP stabilizer may comprise any suitable protein stabilizer, such as a protein stabilizer known in the art. In certain embodiments, an RNP stabilizer comprises 1,2,3-heptanetriol, 2-Amino-2-(hydroxymethyl)-1,3-propanediol (Tris), 3-(1-pyridino)-1-propane sulfonate (NDSB 201), 3-[(3-cholamidopropyl) dimethylammonio]-1-propanesulfonate (CHAPS), 6-aminocaproic acid, adenosine diphosphate (ADP), adenosine triphosphate (ATP), alpha-cyclodextrin, amidosulfobetaine-14 (ASB-14), ammonium acetate, ammonium nitrate, ammonium sulfate, arginine, arginine ethylester, barium chloride, barium iodide, benzamidine HCl, beta-cyclodextrin, beta-mercaptocthanol (BME), biotin, calcium chloride, cesium chloride, cesium sulfate, cetyltrimethylammonium bromide (CTAB), choline chloride, citric acid, cobalt chloride, copper (II) chloride, cyclohexanol, D-sorbitol, dimethylethylammoniumpropane sulfonate (NDSB 195), dithiothritol (DTT), erythritol, ethanol, ethylene glycol, ethylene glycol-bis (βbeta-aminoethyl ether)-N,N,N′,N′-tetraacetic acid (EGTA), ethylenediaminetetraacetic acid (EDTA), formamide, gadolinium bromide, gamma butyrolactone, glucose, glutamic acid, glutamine, glycerol, glycine, glycine betaine, glycine-glycine-glycine, guanidine HCl, guanosine triphosphate (GTP), holmium chloride, imidazole, iron (III) chloride, Jeffamine M-600, lanthanum acetate, lauryl sulfobetaine, lauryldimethylamine N-oxide (LDAO), lithium sulfate, magnesium chloride, magnesium sulfate, manganese chloride, mannitol, N-(2-hydroxyethyl) piperazine-N′-(3-propanesulfonic acid) (EPPS), N-dodecyl beta-D-maltoside (DDM), N-ethylurea, n-hexanol, N-lauryl sarcoside, N-lauryl sarcosine, N-methylformamide, N-methylurea, n-octyl-b-D-glucoside (OG: Octyl glucoside), n-penthanol, nickel chloride, non-detergent sulfo betaine (NDSB), Nonidet P40 (NP40), octyl beta-D-glucopyranoside, poly-L-glutamic acid, polyethylene glycol (for example, PEG 300, PEG 3350, PEG 4000), polyethyleneglycol lauryl ether (Brij 35), polyoxyethylene (2) oleyl ether (Brij 93), polyoxyethylene cetyl ether (Brij 56), polyvinylpyrrolidone 40 (PVP40), potassium chloride, potassium citrate, potassium nitrate, proline, putrescine, spermidine, spermine, riboflavin, samarium bromide, sarcosine, sodium acetate, sodium chloride, sodium dodecyl sulfate (SDS), sodium fluoride, sodium iodide, sodium lauroyl sarcosinate (Sarkosyl), sodium malonate, sodium molybdate, sodium selenite, sodium sulfate, sodium thiocyanate, sucrose, taurine, trehalose, tricine, triethylamine, trimethylamine N-oxide (TMAO), tris (2-carboxyethyl) phosphine (TCEP), Triton X-100, Tween 20, Tween 60, Tween 80, urea, vitamin B12, xylitol, yttrium chloride, yttrium nitrate, zinc chloride, Zwittergent 3-08, Zwittergent 3-14, or a combination thereof. In certain embodiments, the RNP stabilizer comprises a negatively charged polymer. In certain embodiments, the RNP stabilizer comprises poly-L-glutamic acid (PGA) or a suitable alternative. In certain embodiments, provided herein are compositions, methods, and/or kits comprising poly-L-glutamic acid.

The one or more RNP stabilizers can be present at any suitable concentration. In certain embodiments, the one or more RNP stabilizers are present at a concentration of at least 0.01, 0.05, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.5, 2, 2.5, 3, 3.5, 4, or 4.5 and/or not more than 0.05, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.5, 2, 2.5, 3, 3.5, 4, 4.5, or 5 μM per pmol RNP complex, for example 0.01-5 μM per pmol RNP complex, preferably 0.01-3 μM per pmol RNP complex, even more preferably 0.015-2.5 μM per pmol RNP complex, vet more preferably 0.01-1 μM per pmol RNP complex.

The one or more RNP stabilizers can be present at any suitable concentration. In certain embodiments where the one or more RNP stabilizers are a polymer product, the one or more RNP stabilizers are present at a concentration of at least 0.01, 0.05, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.5, 2, 2.5, 3, 3.5, 4, or 4.5 and/or not more than 0.05, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.5, 2, 2.5, 3, 3.5, 4, 4.5, or 5 μg μL⁻¹ per pmol RNP complex, for example 0.01-5 μg μL⁻¹ per pmol RNP complex, preferably 0.01-3 μg μL⁻¹ per pmol RNP complex, even more preferably 0.25-2.5 μg μL⁻¹ per pmol RNP complex, yet more preferably 0.5-1.5 μg μL⁻¹ per pmol RNP complex. In certain embodiments, the polymeric RNP stabilizer comprises PGA.

In certain embodiments, provided herein are compositions, methods, and/or kits comprising one or more additives that inhibit NHEJ, e.g., NHEJ inhibitor. In certain embodiments, the one or more additives that inhibit NHEJ are introduced to the target cell prior to delivery of the nucleic acid-guided nuclease, guide nucleic acid, and/or donor template, or one or more polynucleotides encoding the nucleic acid-guided nuclease, guide nucleic acid, and/or donor template. In certain embodiments, the one or more additives that inhibit NHEJ are introduced to the target cell after delivery of the nucleic acid-guided nuclease, guide nucleic acid, and/or donor template, or one or more polynucleotides encoding the nucleic acid-guided nuclease, guide nucleic acid, and/or donor template. In certain embodiments, the one or more additives that inhibit NHEJ are introduced to the target cell both prior to and after delivery of the nucleic acid-guided nuclease, guide nucleic acid, and/or donor template, or one or more polynucleotides encoding the nucleic acid-guided nuclease, guide nucleic acid, and/or donor template. In certain embodiments, the one or more additives that inhibit NHEJ are introduced into the cell medium, wherein the one or more NHEJ inhibitors can enter the cell.

In certain embodiments, the one or more additives that inhibit NHEJ comprise a molecule that indirectly or directly affects the interaction of p53-binding protein 1 (53BP1) with ubiquitylated histones at double stranded breaks, for example, iP53 or the like. In certain embodiments, the one or more additives that inhibit NHEJ comprise a molecule that directly or indirectly affects the interaction of Ku proteins with DNA, for example, STL127705 or the like. In certain embodiments, the one or more additives that inhibit NHEJ comprise a molecule that directly or indirectly affects the activity of DNA-dependent protein kinases, for example, M3814, KU-0060648, NU7026 or the like. In certain embodiments, the one or more additives that inhibit NHEJ comprise a molecule that directly or indirectly affects the activity of ATM-Rad3-related (ATR) proteins, for example VE-822 or the like. In certain embodiments, the one or more additives that inhibit NHEJ comprise a molecule that directly or indirectly affects the activity of ligases, e.g., ligase IV, for example SCR7 or the like. In certain embodiments, the one or more additives that inhibit NHEJ comprise a molecule that directly or indirectly affects the activity of RAD51 binding to ssDNA, for example RS-1 or the like. In certain embodiments, the one or more additives that inhibit NHEJ comprise a molecule that directly or indirectly affects the activity cell cycle stage progression, for example aphidicolin, mimosin, thymidine, hydroxy urea, nocodazole, ABT-751, XL413, or the like. In certain embodiments, the one or more additives that inhibit NHEJ comprise a molecule that directly or indirectly affects the activity beta-3-adrenergic receptors, for example L755507 or the like. In certain embodiments, the one or more additives that inhibit NHEJ comprise a molecule that directly or indirectly affects the activity of intracellular transport from endoplasmic reticulum (ER) to golgi, for example Brefeldin A or the like. In certain embodiments, the one or more additives that inhibit NHEJ comprise a molecule that directly or indirectly affects the activity histone deacetylases, for example valproic acid (VPA). In certain embodiments, the one or more additives that inhibit NHEJ comprise M3814.

In certain embodiments, the one or more NHEJ inhibitors are present at a concentration of at least 0.1, 0.25, 0.5, 0.75, 1, 1.25, 1.5, 1.75, 2, 2.5, 3, or 4 and/or not more than 0.25, 0.5, 0.75, 1, 1.25, 1.5, 1.75, 2, 2.5, 3, 4, or 5 μM, for example 0.1-5 μM, preferably 0.5-5 μM, even more preferably 1-3 μM, yet more preferably 2 μM. In certain embodiments, the one or more NHEJ inhibitors comprise M3814.

In certain embodiments, the NHEJ inhibitor reduces the activity of NHEJ-based repair, wherein the relative amount of repair via homology-directed repair (HDR) is increased. In certain embodiments, the amount of HDR compared to NHEJ is increased by at least 1.1. 1.15, 1.2. 1.25, 1.3, 1.35, 1.4, 1.45, 1.5, 1.55, 1.6, 1.65, 1.7, 1.75, 1.8, 1.85, 1.9, 1.95, 2, 2.25, 2.5, 2.75, 3, 4, 5, 6, 7, 8, or 9-fold and/or not more than 1.15, 1.2, 1.25, 1.3, 1.35, 1.4, 1.45, 1.5, 1.55, 1.6, 1.65, 1.7, 1.75, 1.8, 1.85, 1.9, 1.95, 2, 2.25, 2.5, 2.75, 3, 4, 5, 6, 7, 8, 9, or 10-fold increased editing via homology directed repair (HDR) as compared to editing via NHEJ in cells treated with the one or more NHEJ inhibitors as compared to those not treated with one or more NHEJ inhibitors, for example 1.1-10-fold increased editing, preferably 1.1-5-fold increased editing, even more preferably 1.1-3-fold increased editing, yet more preferably 1.1-2-fold increased editing. In certain embodiments, the amount of INDEL formation due to NHEJ as measured by sequencing is reduced by at least 1.1, 1.15, 1.2, 1.25, 1.3, 1.35, 1.4, 1.45, 1.5, 1.55, 1.6, 1.65, 1.7, 1.75, 1.8, 1.85, 1.9, 1.95, 2, 2.25, 2.5, 2.75, 3, 4, 5, 6, 7, 8, or 9-fold and/or not more than 1.15, 1.2. 1.25, 1.3. 1.35, 1.4, 1.45, 1.5, 1.55, 1.6, 1.65, 1.7, 1.75, 1.8, 1.85, 1.9, 1.95, 2, 2.25, 2.5, 2.75, 3, 4, 5, 6, 7, 8, 9, or 10-fold reduced INDEL formation due to NHEJ as compared to an untreated control, for example 1.1-10-fold reduced INDEL formation, preferably 1.1-5-fold reduced INDEL formation, even more preferably 1.1-3-fold reduced INDEL formation, yet more preferably 1.1-2-fold reduced INDEL formation. Any suitable sequencing method known in the art may be used to determine the relative types of edits generated following treatment.

In certain embodiments, provided herein are compositions, methods, and/or kits comprising nucleic acid-guided nucleases. In certain embodiments, provided herein are compositions, methods, and/or kits comprising engineered nucleic acid-guided nucleases. In certain embodiments, provided herein are compositions, methods, and/or kits wherein the nuclease comprises a Cas nuclease. In certain embodiments, provided herein are compositions, methods, and/or kits wherein the nuclease comprises a Class 1 or Class 2 Cas nuclease. In certain embodiments, provided herein are compositions, methods, and/or kits wherein the nuclease comprises a Type V nuclease. In certain embodiments, provided herein are compositions, methods, and/or kits wherein the nuclease comprises a Type V-A, V-B, V-C, V-D, or V-E nuclease. In certain embodiments, provided herein are compositions, methods, and/or kits wherein the nuclease comprises a Type V-A nuclease. In certain embodiments, provided herein are compositions, methods, and/or kits wherein the nuclease comprises a MAD, ABW, or ART nuclease. In certain embodiments, provided herein are compositions, methods, and/or kits wherein the nuclease comprises a MAD1, MAD2, MAD3, MAD4, MAD5, MAD6, MAD7, MAD8, MAD9, MAD10, MAD11, MAD12, MAD13, MAD14, MAD15, MAD16, MAD17, MAD18, MAD19, or MAD20 nuclease. In certain embodiments, provided herein are compositions, methods, and/or kits wherein the nuclease comprises an ART1, ART2, ART3, ART4, ART5, ART6, ART7, ART8, ART9, ART10, ART11, ART11*, ART12, ART13, ART14, ART15, ART16, ART17, ART18, ART19, ART20, ART21, ART22, ART23, ART24, ART25, ART26, ART27, ART28, ART29, ART30, ART31, ART32, ART33, ART34, or ART35 nuclease. In certain embodiments, provided herein are compositions, methods, and/or kits wherein the nuclease comprises a MAD2, MAD7, ART11, ART11*, or ART2 nuclease. In certain embodiments, provided herein are compositions, methods, and/or kits wherein the nuclease comprises one or more nuclear localization signals. In certain embodiments, provided herein are compositions, methods, and/or kits the nuclease comprises 1 or 4 nuclear localization signals, such as 1-4 NLS at the carboxy terminus, 1-4 NLS at the amino terminus, or a combination thereof. Additional nucleases and modifications thereof may be found in the Cas nuclease section below.

In certain embodiments, provided herein are compositions, methods, and/or kits wherein the relative amount (e.g., proportion) of gNA to nuclease results in improved editing efficiencies. In certain embodiments, provided herein are compositions, methods, and/or kits wherein the proportion of gNA to nuclease is at least 1, 1.05 1.1, 1.15, 1.2, 1.25, 1.3, 1.35, 1.4, 1.45, 1.5, 1.55, 1.6, 1.65, 1.7, 1.75, 1.8, 1.85, 1.9, or 1.95 and/or not more than 1.05 1.1, 1.15, 1.2, 1.25, 1.3, 1.35, 1.4, 1.45, 1.5, 1.55, 1.6, 1.65, 1.7, 1.75, 1.8, 1.85, 1.9, 1.95 or 2 parts for every part of nuclease, for example, 1-2 parts of gNA for every part of nuclease, preferably, 1.15-1.85 parts of gNA for every part of nuclease, even more preferably 1.25-1.75 parts of gNA for every part of nuclease, yet more preferably 1.5 parts of gNA for every part of nuclease. In certain embodiments, provided herein are compositions, methods, and/or kits the gNA and nuclease are present at 150:100 or 75:50 pmol respectively.

In certain embodiments, provided herein are compositions, methods, and/or kits wherein the amount of donor template delivered to the cell results affects editing efficiencies. In certain embodiments, provided herein are compositions, methods, and/or kits wherein the donor template is present at a concentration of at least 0.05, 0.01, 0.2, 0.3, 0.4, 0.5, 0.6, 0.7, 0.8, 0.9, 1, 1.25, 1.5, 1.75, 2, 3, or 4, and/or no more than 0.01, 0.2, 0.3, 0.4, 0.5, 0.6, 0.7, 0.8, 0.9, 1, 1.25, 1.5, 1.75, 2, 3, 4, or 5 μg μL⁻¹, for example 0.01-5 μg μL⁻¹, preferably 0.01-3 μg μL⁻¹, even more preferably 0.3-3 μg μL⁻¹, yet even more preferably 0.5-1.5 μg μL⁻¹.

In certain embodiments, provided herein are compositions comprising a nucleic acid-guided nuclease system and at least one additive that stabilizes the nucleic acid-guided nucleases. In certain embodiments, the nucleic acid-guided nuclease system comprises a naturally occurring system. In certain embodiments, the nucleic acid-guided nuclease system comprises an engineered, non-naturally occurring system. In certain embodiments, provided herein is a composition comprising one or more nucleases system comprising: a nucleic acid-guided nuclease; and a guide nucleic acid (gNA) compatible with and capable of binding to and activating the nucleic acid-guided nuclease, wherein the gNA comprises: a targeter nucleic acid comprising a targeter stem sequence and a spacer sequence, wherein the spacer sequence is complementary to a target nucleotide sequence within a target polynucleotide, for example a target polynucleotide of a genome of a human target cell; and a modulator nucleic acid comprising a modulator stem sequence complementary to the targeter stem sequence, and, optionally, a 5′ sequence; and at least one additive that stabilizes the nucleic acid-guided nuclease system. In certain embodiments, the composition comprises any nuclease disclosed herein in the Cas nuclease section. In certain embodiments, the composition comprises a single guide nucleic acid. In certain embodiments, the composition comprises a dual guide nucleic acid as disclosed herein in the Guide nucleic acids section. In certain embodiments, the composition comprises a guide nucleic acid comprising a spacer sequence comprising any one of SEQ ID NOs: 86-384 as shown in Table 5. In certain embodiments, the guide nucleic acid comprises one or more chemical modifications as disclosed herein in the gNA modifications section. In certain embodiments, the composition further comprises a donor template as disclosed herein in the Donor templates section. In certain embodiments, the composition is introduced into one or more cells, wherein the composition can bind to a target sequence within a target polynucleotide within the genome of a human target cell and generate a strand break in at least one strand at or near the target sequence. In certain embodiments, the NHEJ inhibitor is added to the one or more human target cells prior to or after delivery of the composition. In certain embodiments, at least a portion of the donor template is introduced into the target polynucleotide at or near the strand break via an innate cell repair mechanism. In certain embodiments the innate repair mechanism comprises homology directed repair (HDR), e.g., homologous recombination.

In certain embodiments, provided herein are compositions comprising one or more human target cells comprising at least one additive that reduces non-homologous end joining (NHEJ). In certain embodiments, provided herein are compositions further comprising a nucleic acid-guided nuclease as disclosed herein in Cas nuclease section. In certain embodiments, provided herein is a composition comprising: a nucleic acid-guided nuclease capable of binding to a compatible guide nucleic acid (gNA) comprising a spacer sequence complementary to a target nucleotide sequence within a target polynucleotide, e.g., a target polynucleotide of a genome of a human target cell and generating a strand break in one or both strands of the target polynucleotide: one or more human target cells; and at least one additive that reduces non-homologous end joining (NHEJ)-based DNA repair. In certain embodiments provided herein is a composition comprising a human cell comprising: a nuclease capable of binding to a compatible guide nucleic acid (gNA) comprising a spacer sequence complementary to a target nucleotide sequence within a target polynucleotide of a genome of the human cell and generating a strand break in one or both strands of the target polynucleotide; and at least one additive that reduces non-homologous end joining (NHEJ)-based DNA repair. In certain embodiments, the composition further comprises a guide nucleic acid as disclosed herein in the Guide nucleic acids section. In certain embodiments, the composition comprises a guide nucleic acid comprising a spacer sequence comprising any one of SEQ ID NOs: 86-384 as shown in Table 5. In certain embodiments, the guide nucleic acid comprises one or more chemical modifications as disclosed herein in the gNA modifications section. In certain embodiments, the nuclease forms a nucleic acid-guided nuclease complex with the guide nucleic acid. In certain embodiments, the composition further comprises a donor template as disclosed herein in the Donor templates section. In certain embodiments, the nuclease complex can bind to a target sequence within a target polynucleotide within the genome of a human target cell and generate a strand break in at least one strand at or near the target sequence. In certain embodiments, the NHEJ inhibitor is added to the one or more human target cells prior to or after delivery of the composition. In certain embodiments, at least a portion of the donor template is introduced into the target polynucleotide at or near the strand break via an innate cell repair mechanism. In certain embodiments the innate repair mechanism comprises homology directed repair (HDR), e.g., homologous recombination.

In certain embodiments, provided herein are methods. In certain embodiments, provided herein are methods for engineering cells. In certain embodiments, provided herein are methods for engineering human cells. In certain embodiments, provided herein are methods for efficiently engineering human cells. In certain embodiments, provided herein is a method for editing a target polynucleotide in the genome of a human target cell comprising one or more of steps (A) to (G), wherein step (A) comprises forming the nuclease complex by combining one or more nucleases with one or more guide nucleic acids and/or one or more RNP stabilizers: step (B) comprises delivering the nuclease system to the human target cell: step (C) comprises delivering one or more donor templates to the human target cell: step (D) comprises contacting the target polynucleotide with a nuclease system comprising: a nucleic acid-guided nuclease; and a guide nucleic acid (gNA) compatible with and capable of binding to and activating the nucleic acid-guided nuclease, wherein the gNA comprises: a targeter nucleic acid comprising a targeter stem sequence and a spacer sequence, wherein the spacer sequence is complementary to a target nucleotide sequence within a target polynucleotide, for example a target polynucleotide of a genome of a human target cell; and a modulator nucleic acid comprising a modulator stem sequence complementary to the targeter stem sequence, and, optionally, a 5′ sequence: step (E) comprises contacting the cell with at least one additive that reduces non-homologous end joining (NHEJ)-based DNA repair: step (F) comprises growing the cell in a suitable growth medium: step (G) isolating one or more cells that demonstrate the genotype and/or phenotype of interest. In certain embodiments, any number of steps (A) through (G) may be performed in any order. In certain embodiments, the one or more steps (A) through (G) may be performed on the same population of cells. In certain embodiments, the one or more steps (A) through (G) may be performed on the progeny of a first set of cells treated with the one or more steps (A) through (G).

In certain embodiments, the method comprises the following steps and order: step (A) is performed wherein the gNA is combined with the RNP stabilizer prior to addition of the nuclease to form a stabilized nucleic acid-guided nuclease complex; step (B) and step (C) are performed sequentially such that the one or more nucleic acid-guided nuclease complexes are combined with the one or more donor templates and delivered to the one or more human target cells; step (D); step (E) wherein the one or more NHEJ inhibitors are added to the cell recovery medium; step (F).

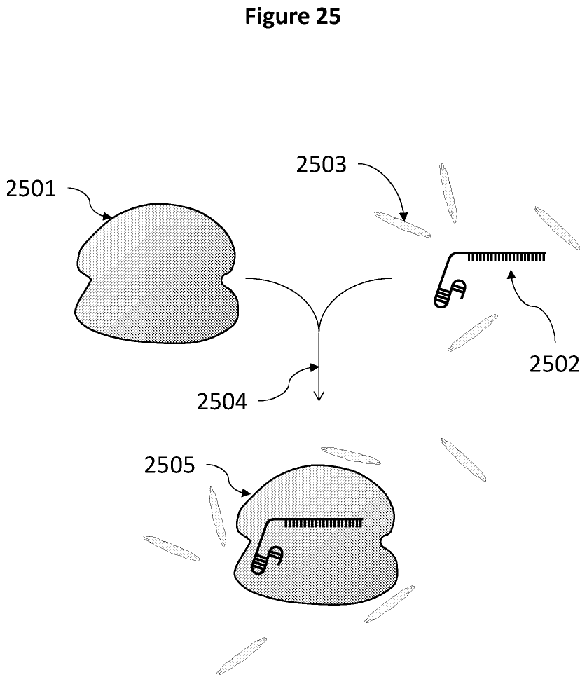

Step (A) is illustrated in FIG. 25. FIG. 25 shows the combination of a guide nucleic acid (2502) with one or more RNP stabilizers (2503). The nuclease (2501) is combined (2504) with the gNA-RNP stabilizer mixture, whereby a stabilized nucleic acid-guided nuclease complex (2505) is formed. The gNA molecule can comprise either a single or dual guide nucleic acid. A single gNA is shown in FIG. 25 for illustrative purposes only.

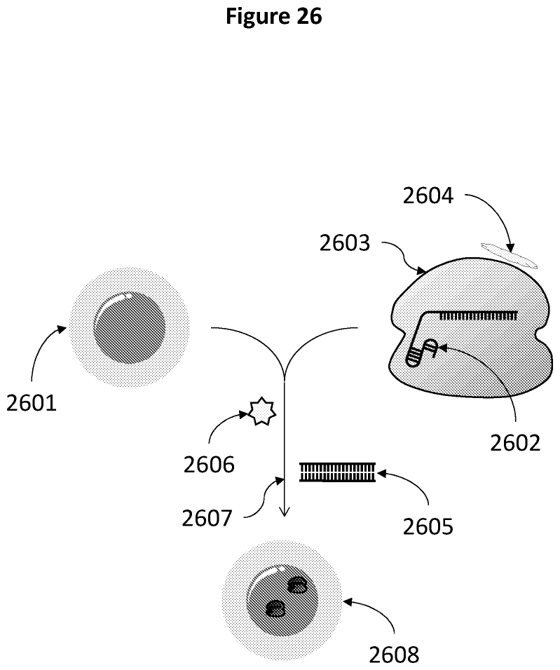

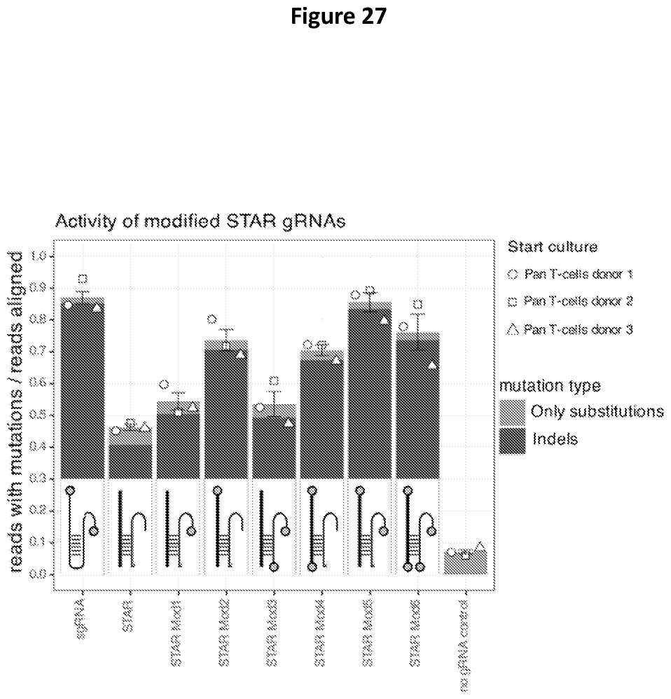

Steps (B) through (E) are illustrated in FIG. 26. FIG. 26 shows the delivery (2607) of the stabilized RNP complex (2603) comprising a nuclease, one or more RNP stabilizer (2604), and a guide nucleic acid (2602) along with, optionally, one or more donor templates (2605) to one or more human target cells (2601), resulting in a cell comprising a one or more nuclease complex and/or one or more donor templates (2608). The one or more NHEJ inhibitors (2606) may be added before or after delivery of the nucleic acid-guided nuclease complex and/or the one or more donor templates.

In certain embodiments, the human cell comprises an immune cell or a stem cell. In certain embodiments, the immune cell comprises a neutrophil, eosinophil, basophil, mast cell, monocyte, macrophage, dendritic cell, natural killer cell, or a lymphocyte. In certain embodiments, the immune cell comprises a T cell. In certain embodiments, the T cell comprises a CAR-T cell. In certain embodiments, the stem cell comprises a human pluripotent, multipotent stem cell, embryonic stem cell, induced pluripotent stem cell, CD34+ stem cell, or hematopoietic stem cell. In certain embodiments, the human cell is allogeneic, i.e., a cell that provokes little or no immune response when introduced into an allogeneic host and produces little or no graft versus host response.

### III. Engineered Non-Naturally-Occurring Dual Guide CRISPR-Cas Systems

A CRISPR-Cas system generally comprises a Cas protein and one or more guide nucleic acids (gNAs). The Cas protein can be directed to a specific location in a double-stranded DNA target by recognizing a protospacer adjacent motif (PAM) in the non-target strand of the DNA, and the one or more guide nucleic acids can be directed to a specific location by hybridizing with a target nucleotide sequence, also referred to herein as a target sequence, in the target strand of the target polynucleotide. Typically, both PAM recognition and target nucleotide sequence hybridization are required for stable binding of a CRISPR-Cas complex to the DNA target and, if the Cas protein has an effector function (e.g., nuclease activity), activation of the effector function. As a result, when creating a CRISPR-Cas system, a guide nucleic acid can be designed to comprise a nucleotide sequence called a spacer sequence that is at least partially complementary to and can hybridize with a target nucleotide sequence, where target nucleotide sequence is located adjacent to a PAM in an orientation operable with the Cas protein. It has been observed that not all CRISPR-Cas systems designed by these criteria are equally effective. The larger polynucleotide in which a target nucleotide sequence is located may be referred to as a target polynucleotide: e.g., a chromosome or other genomic DNA, or portion thereof, or any other suitable polynucleotide within which a target nucleotide sequence is located. The target polynucleotide in double stranded DNA comprises two strands. The strand of the DNA duplex to which the spacer sequence is complementary herein is called the “target strand,” while the strand to which the spacer sequence shares sequence identity herein is called the “non-target strand.”

Two distinct classes of CRISPR-Cas systems have been identified. Class 1 CRISPR-Cas systems utilize multi-protein effector complexes, whereas class 2 CRISPR-Cas systems utilize single-protein effectors (see, Makarova et al. (2017) CELL, 168:328). Among the types of class 2 CRISPR-Cas systems, type II and type V systems typically target DNA and type VI systems typically target RNA (id.). Naturally occurring type II effector complexes include Cas9, CRISPR RNA (crRNA), and trans-activating CRISPR RNA (tracrRNA), but the crRNA and tracrRNA can be fused as a single guide RNA in an engineered system for simplicity (see, Wang et al. (2016) ANNU. REV. BIOCHEM., 85:227). Certain naturally occurring type V systems, such as type V-A, type V-C, and type V-D systems, do not require tracrRNA and use crRNA alone as the guide for cleavage of target DNA (see, Zetsche et al. (2015) CELL, 163:759; Makarova et al. (2017) CELL, 168:328.

Naturally occurring type II CRISPR-Cas systems (e.g., CRISPR-Cas9 systems) generally comprise two guide nucleic acids, called crRNA and tracrRNA, which form a complex by nucleotide hybridization. Single guide nucleic acids capable of activating type II Cas nucleases have been developed, for example, by linking the crRNA and the tracrRNA (see, e.g., U.S. Pat. Nos. 10,266,850 and 8,906,616). Naturally occurring type II Cas proteins comprise a RuvC-like nuclease domain and an HNH endonuclease domain, and recognize a 3′ G-rich PAM located immediately downstream from the target nucleotide sequence, the orientation determined using the non-target strand (i.e., the strand not hybridized with the spacer sequence) as the coordinate. The CRISPR-Cas systems cleave a double-stranded DNA to generate a blunt end. The cleavage site is generally 3-4 nucleotides upstream from the PAM on the non-target strand.

Naturally occurring Type V-A, Type V-C, and Type V-D CRISPR-Cas systems lack a tracrRNA and rely on a single crRNA to guide the CRISPR-Cas complex to the target polynucleotide. Dual guide nucleic acids capable of activating type V-A, type V-C, or type V-D Cas nucleases have been developed, for example, by splitting the single crRNA into a targeter nucleic acid and a modulator nucleic acid (see, e.g., International (PCT) Application Publication No. WO 2021/067788). Naturally occurring type V-A Cas proteins comprise a RuvC-like nuclease domain but lack an HNH endonuclease domain, and recognize a 5′ T-rich PAM located immediately upstream from the target nucleotide sequence, the orientation determined using the non-target strand (i.e., the strand not hybridized with the spacer sequence) as the coordinate. These CRISPR-Cas systems cleave a double-stranded DNA to generate a staggered double-stranded break rather than a blunt end. The cleavage site is distant from the PAM site (e.g., separated by at least 10, 11, 12, 13, 14, or 15 nucleotides downstream from the PAM on the non-target strand and/or separated by at least 15, 16, 17, 18, or 19 nucleotides upstream from the sequence complementary to PAM on the target strand).

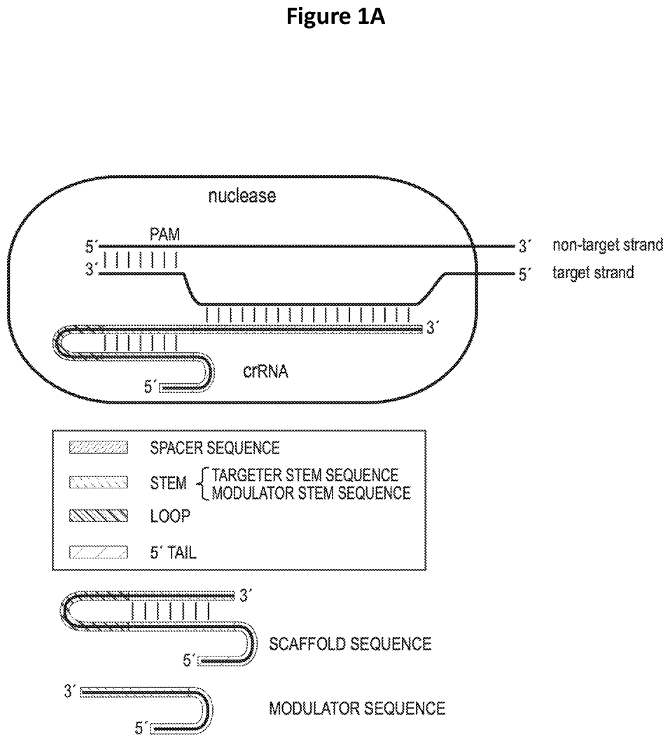

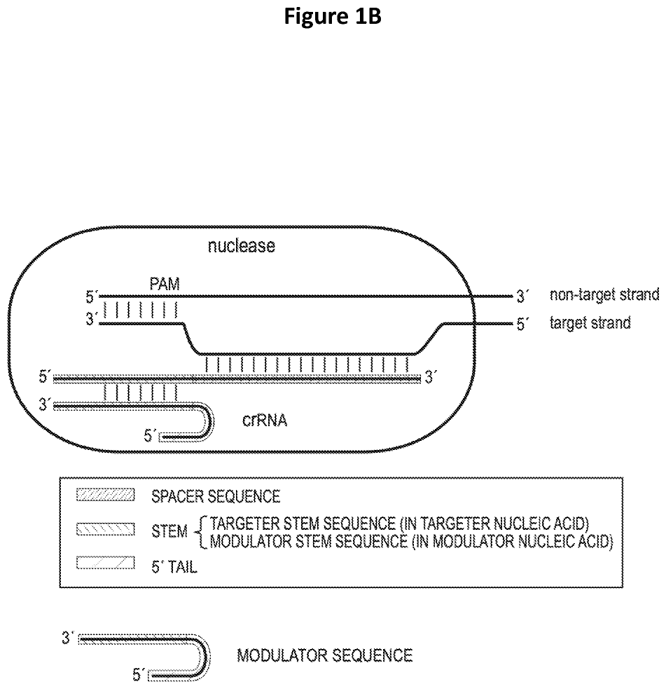

Elements in an exemplary single guide CRISPR Cas system, e.g., a type V-A CRISPR-Cas system, are shown in FIG. 1A. The single gNA can also be called a “crRNA” or “single gRNA” where it is present in the form of an RNA. It can comprise, from 5′ to 3′, an optional 5′ sequence, e.g., a tail, a modulator stem sequence, a loop, a targeter stem sequence complementary to the modulator stem sequence, and a spacer sequence that is at least partially complementary to and can hybridize with a target sequence in the target strand of the target polynucleotide. Where a 5′ tail is present, the sequence including the 5′ tail and the modulator stem sequence can also be called a “modulator sequence” herein. A fragment of the single guide nucleic acid from the optional 5′ tail to the targeter stem sequence, also called a “scaffold sequence” herein, bind the Cas protein. In addition, the PAM in the non-target strand of the target DNA binds the Cas protein.

Elements in an exemplary dual guide type CRISPR Cas system, e.g., a dual guide type V-A CRISPR-Cas system are shown in FIG. 1B. The first guide nucleic acid, which can be called a “modulator nucleic acid” herein, comprises, from 5′ to 3′, an optional 5′ tail and a modulator stem sequence. Where a 5′ tail is present, the sequence including the 5′ tail and the modulator stem sequence can also called a “modulator sequence” herein. The second guide nucleic acid, which can be called “targeter nucleic acid” herein, comprises, from 5′ to 3″, a targeter stem sequence complementary to the modulator stem sequence and a spacer sequence that is at least partially complementary to and can hybridize with the target sequence in the target strand of the target polynucleotide. The duplex between the modulator stem sequence and the targeter stem sequence, plus the optional 5′ tail, constitute a structure that binds the Cas protein. In addition, the PAM in the non-target strand of the target DNA binds the Cas protein. It is understood that, in a dual gNA, e.g., dual gRNA, the targeter nucleic acid and the modulator nucleic acid, while not in the same nucleic acids, i.e., not linked end-to-end through a traditional internucleotide bond, can be covalently conjugated to each other through one or more chemical modifications introduced into these nucleic acids, thereby increasing the stability of the double-stranded complex and/or improving other characteristics of the system.

The terms “targeter stem sequence” and “modulator stem sequence,” as used herein, can refer to a pair of nucleotide sequences in one or more guide nucleic acids that hybridize with each other. When a targeter stem sequence and a modulator stem sequence are contained in a single guide nucleic acid, the targeter stem sequence is proximal to a spacer sequence designed to hybridize with a target nucleotide sequence, and the modulator stem sequence is proximal to the targeter stem sequence. When a targeter stem sequence and a modulator stem sequence are in separate nucleic acids, the targeter stem sequence is in the same nucleic acid as a spacer sequence designed to hybridize with a target nucleotide sequence. In a CRISPR-Cas system that naturally includes separate crRNA and tracrRNA (e.g., a type II system), the duplex formed between the targeter stem sequence and the modulator stem sequence corresponds to the duplex formed between the crRNA and the tracrRNA. In a CRISPR-Cas system that naturally includes a single crRNA but no tracrRNA (e.g., a type V-A system), the duplex formed between the targeter stem sequence and the modulator stem sequence corresponds to the stem portion of a stem-loop structure in the scaffold sequence of the crRNA. It is understood that 100% complementarity is not required between the targeter stem sequence and the modulator stem sequence. In a type V-A CRISPR-Cas system, however, the targeter stem sequence is typically 100% complementary to the modulator stem sequence.

### A. Cas Proteins

A guide nucleic acid, either as a single guide nucleic acid alone (targeter and modulator nucleic acids are part of a single polynucleotide) or as a dual gNA comprising separate targeter nucleic acid used in combination with a cognate modulator nucleic acid, is capable of binding a CRISPR Associated (Cas) protein, e.g., a Cas nuclease. In certain embodiments, the guide nucleic acid, either as a single guide nucleic acid alone (targeter and modulator nucleic acids are part of a single polynucleotide) or as a dual gNA comprising separate targeter nucleic acid used in combination with a cognate modulator nucleic acid, is capable of activating a Cas nuclease. A gNA capable of activating a particular Cas nuclease is said to be “compatible” with the Cas nuclease; a Cas nuclease capable of being activated by a particular gNA is said to be “compatible” with the gNA.

The terms “CRISPR-Associated protein,” “Cas protein,” and “Cas,” as used interchangeably herein, can refer to a naturally occurring Cas protein or an engineered Cas protein. Non-limiting examples of Cas protein engineering include but are not limited to mutations and modifications of the Cas protein that alter the activity of the Cas, alter the PAM specificity, broaden the range of recognized PAMs, and/or reduce the ability to modify one or more off-target loci as compared to a corresponding unmodified Cas. In certain embodiments, the altered activity of engineered Cas comprises altered ability (e.g., specificity or kinetics) to bind a naturally occurring gNA, e.g., gRNA or engineered gNA, e.g., gRNA, altered ability (e.g., specificity or kinetics) to bind a target nucleotide sequence, altered processivity of nucleic acid scanning, and/or altered effector (e.g., nuclease) activity. A Cas protein having nuclease activity can be referred to as a “CRISPR-Associated nuclease” or “Cas nuclease,” or simply “nuclease,” as used interchangeably herein.

In certain embodiments, the Cas protein is a type V-A, type V-C, or type V-D Cas protein. In certain embodiments, the Cas protein is a type V-A Cas protein. In other embodiments, the Cas protein is a type II Cas protein, e.g., a Cas9 protein.

In certain embodiments, a type V-A Cas nucleases comprises Cpf1. Cpf1 proteins are known in the art and are described, e.g., in U.S. Pat. Nos. 9,790,490 and 10,113,179. Cpf1 orthologs can be found in various bacterial and archaeal genomes. For example, in certain embodiments, the Cpf1 protein is derived from Francisella novicida U112 (Fn), Acidaminococcus sp. BV3L6 (As), Lachnospiraceae bacterium ND2006 (Lb), Lachnospiraceae bacterium MA2020 (Lb2), Candidatus Methanoplasma termitum (CMt), Moraxella bovoculi 237 (Mb), Porphyromonas crevioricanis (Pc), Prevotella disiens (Pd), Francisella tularensis 1, Francisella tularensis subsp. novicida, Prevotella albensis, Lachnospiraceae bacterium MC2017 1, Butyrivibrio proteoclasticus, Peregrinibacteria bacterium GW2011_GWA2_33_10, Parcubacteria bacterium GW2011_GWC2 44 17, Smithella sp. SCADC, Eubacterium eligens, Leptospira inadai, Porphyromonas macacae. Prevotella bryantii. Proteocatella sphenisci. Anaerovibrio sp. RM50, Moraxella caprae. Lachnospiraceae bacterium COE1, or Eubacterium coprostanoligenes.

In certain embodiments, a type V-A Cas nuclease comprises AsCpf1 or a variant thereof. In certain embodiments, a type V-A Cas protein comprises an amino acid sequence at least 30%, 40%, 50%, 60%, 70%, 80%, 85%, 90%, 91%, 92%, 93%, 94%, 95%, 96%, 97%, 98%, 99%, or 100% identical to the amino acid sequence set forth in SEQ ID NO: 3 of International (PCT) Application Publication No. WO 2021/158918. In certain embodiments, a type V-A Cas protein comprises the amino acid sequence set forth in SEQ ID NO: 3 of International (PCT) Application Publication No. WO 2021/158918.

In certain embodiments, a type V-A Cas nuclease comprises LbCpf1 or a variant thereof. In certain embodiments, a type V-A Cas protein comprises an amino acid sequence at least 30%, 40%, 50%, 60%, 70%, 80%, 85%, 90%, 91%, 92%, 93%, 94%, 95%, 96%, 97%, 98%, 99%, or 100% identical to the amino acid sequence set forth in SEQ ID NO: 4 of International (PCT) Application Publication No. WO 2021158918. In certain embodiments, a type V-A Cas protein comprises the amino acid sequence set forth in SEQ ID NO: 4 of International (PCT) Application Publication No. WO 2021/158918.

In certain embodiments, a type V-A Cas nuclease comprises FnCpf1 or a variant thereof. In certain embodiments, a type V-A Cas protein comprises an amino acid sequence at least 30%, 40%, 50%, 60%, 70%, 80%, 85%, 90%, 91%, 92%, 93%, 94%, 95%, 96%, 97%, 98%, 99%, or 100% identical to the amino acid sequence set forth in SEQ ID NO: 5 of International (PCT) Application Publication No. WO 2021158918. In certain embodiments, a type V-A Cas protein comprises the amino acid sequence set forth in SEQ ID NO: 5 of International (PCT) Application Publication No. WO 2021/158918.

In certain embodiments, a type V-A Cas nuclease comprises Prevotella bryantii Cpf1 (PbCpf1) or a variant thereof. In certain embodiments, a type V-A Cas protein comprises an amino acid sequence at least 30%, 40%, 50%, 60%, 70%, 80%, 85%, 90%, 91%, 92%, 93%, 94%, 95%, 96%, 97%, 98%, 99%, or 100% identical to the amino acid sequence set forth in SEQ ID NO: 6 of International (PCT) Application Publication No. WO 2021/158918. In certain embodiments, a type V-A Cas protein comprises the amino acid sequence set forth in SEQ ID NO: 6 of International (PCT) Application Publication No. WO 2021/158918.

In certain embodiments, a type V-A Cas nuclease comprises Proteocatella sphenisci Cpf1 (PsCpf1) or a variant thereof. In certain embodiments, a type V-A Cas protein comprises an amino acid sequence at least 30%, 40%, 50%, 60%, 70%, 80%, 85%, 90%, 91%, 92%, 93%, 94%, 95%, 96%, 97%, 98%, 99%, or 100% identical to the amino acid sequence set forth in SEQ ID NO: 7 of International (PCT) Application Publication No. WO 2021158918. In certain embodiments, a type V-A Cas protein comprises the amino acid sequence set forth in SEQ ID NO: 7 of International (PCT) Application Publication No. WO 2021/158918.

In certain embodiments, a type V-A Cas nuclease comprises Anaerovibrio sp. RM50 Cpf1 (As2Cpf1) or a variant thereof. In certain embodiments, a type V-A Cas protein comprises an amino acid sequence at least 30%, 40%, 50%, 60%, 70%, 80%, 85%, 90%, 91%, 92%, 93%, 94%, 95%, 96%, 97%, 98%, 99%, or 100% identical to the amino acid sequence set forth in SEQ ID NO: 8 of International (PCT) Application Publication No. WO 2021158918. In certain embodiments, a type V-A Cas protein comprises the amino acid sequence set forth in SEQ ID NO: 8 of International (PCT) Application Publication No. WO 2021/158918.

In certain embodiments, a type V-A Cas nuclease comprises Moraxella caprae Cpf1 (McCpf1) or a variant thereof. In certain embodiments, a type V-A Cas protein comprises an amino acid sequence at least 30%, 40%, 50%, 60%, 70%, 80%, 85%, 90%, 91%, 92%, 93%, 94%, 95%, 96%, 97%, 98%, 99%, or 100% identical to the amino acid sequence set forth in SEQ ID NO: 9 of International (PCT) Application Publication No. WO 2021/158918. In certain embodiments, a type V-A Cas protein comprises the amino acid sequence set forth in SEQ ID NO: 9 of International (PCT) Application Publication No. WO 2021/158918.

In certain embodiments, a type V-A Cas nuclease comprises Lachnospiraceae bacterium COE1 Cpf1 (Lb3Cpf1) or a variant thereof. In certain embodiments, a type V-A Cas protein comprises an amino acid sequence at least 30%, 40%, 50%, 60%, 70%, 80%, 85%, 90%, 91%, 92%, 93%, 94%, 95%, 96%, 97%, 98%, 99%, or 100% identical to the amino acid sequence set forth in SEQ ID NO: 10 of International (PCT) Application Publication No. WO 2021158918. In certain embodiments, a type V-A Cas protein comprises the amino acid sequence set forth in SEQ ID NO: 10 of International (PCT) Application Publication No. WO 2021/158918.

In certain embodiments, a type V-A Cas nuclease comprises Eubacterium coprostanoligenes Cpf1 (EcCpf1) or a variant thereof. In certain embodiments, a type V-A Cas protein comprises an amino acid sequence at least 30%, 40%, 50%, 60%, 70%, 80%, 85%, 90%, 91%, 92%, 93%, 94%, 95%, 96%, 97%, 98%, 99%, or 100% identical to the amino acid sequence set forth in SEQ ID NO: 11 of International (PCT) Application Publication No. WO 2021158918. In certain embodiments, a type V-A Cas protein comprises the amino acid sequence set forth in SEQ ID NO: 11 of International (PCT) Application Publication No. WO 2021/158918.

In certain embodiments, a type V-A Cas nuclease is not Cpf1. In certain embodiments, a type V-A Cas nuclease is not AsCpf1.

In certain embodiments, a type V-A Cas nuclease comprises MAD1, MAD2, MAD3, MAD4, MAD5, MAD6, MAD7, MAD8, MAD9, MAD10, MAD11, MAD12, MAD13, MAD14, MAD15, MAD16, MAD17, MAD18, MAD19, or MAD20, or variants thereof. MAD1-MAD20 are known in the art and are described in U.S. Pat. No. 9,982,279.

In certain embodiments, a type V-A Cas nuclease comprises MAD7 or a variant thereof. In certain embodiments, a type V-A Cas protein comprises an amino acid sequence at least 30%, 40%, 50%, 60%, 70%, 80%, 85%, 90%, 91%, 92%, 93%, 94%, 95%, 96%, 97%, 98%, 99%, or 100% identical to the amino acid sequence set forth in SEQ ID NO: 37. In certain embodiments, a type V-A Cas protein comprises the amino acid sequence set forth in SEQ ID NO: 37.

MAD7
(SEQ ID NO: 37)
MNNGTNNFQNFIGISSLQKTLRNALIPTETTQQFIVKNGIIKEDE
LRGENRQILKDIMDDYYRGEISETLSSIDDIDWTSLFEKMEIQLK
NGDNKDTLIKEQTEYRKAIHKKFANDDRFKNMESAKLISDILPEF
VIHNNNYSASEKEEKTQVIKLESRFATSFKDYFKNRANCESADDI
SSSSCHRIVNDNAEIFFSNALVYRRIVKSLSNDDINKISGDMKDS
LKEMSLEEIYSYEKYGEFITQEGISFYNDICGKVNSFMNLYCQKN
KENKNLYKLQKLHKQILCIADTSYEVPYKFESDEEVYQSVNGELD
NISSKHIVERLRKIGDNYNGYNLDKIYIVSKFYESVSQKTYRDWE
TINTALEIHYNNILPGNGKSKADKVKKAVKNDLQKSITEINELVS
NYKLCSDDNIKAETYIHEISHILNNFEAQELKYNPEIHLVESELK
ASELKNVLDVIMNAFHWCSVEMTEELVDKDNNFYAELEEIYDEIY
PVISLYNLVRNYVTQKPYSTKKIKLNEGIPTLADGWSKSKEYSNN
AIILMRDNLYYLGIFNAKNKPDKKIIEGNTSENKGDYKKMIYNLL
PGPNKMIPKVFLSSKTGVETYKPSAYILEGYKQNKHIKSSKDEDI
TFCHDLIDYFKNCIAIHPEWKNFGFDESDTSTYEDISGFYREVEL
QGYKIDWTYISEKDIDLLQEKGQLYLFQIYNKDESKKSTGNDNLH
TMYLKNLFSEENLKDIVLKLNGEAEIFFRKSSIKNPIIHKKGSIL
VNRTYEAEEKDQFGNIQIVRKNIPENIYQELYKYFNDKSDKELSD
EAAKLKNVVGHHEAATNIVKDYRYTYDKYFLHMPITINFKANKTG
FINDRILQYIAKEKDLHVIGIDRGERNLIYVSVIDTCGNIVEQKS
ENIVNGYDYQIKLKQQEGARQIARKEWKEIGKIKEIKEGYLSLVI
HEISKMVIKYNAIIAMEDLSYGFKKGREKVERQVYQKFETMLINK
LNYLVEKDISITENGGLLKGYQLTYIPDKLKNVGHQCGCIFYVPA
AYTSKIDPTTGFVNIFKFKDLTVDAKREFIKKEDSIRYDSEKNLF
CFTEDYNNFITQNTVMSKSSWSVYTYGVRIKRRFVNGRESNESDT
IDITKDMEKTLEMTDINWRDGHDLRQDIIDYEIVQHIFEIFRLTV
QMRNSLSELEDRDYDRLISPVLNENNIFYDSAKAGDALPKDADAN
GAYCIALKGLYEIKQITENWKEDGKFSRDKLKISNKDWEDFIQNK
RYL

In certain embodiments, a type V-A Cas nuclease comprises MAD2 or a variant thereof. In certain embodiments, a type V-A Cas protein comprises an amino acid sequence at least 30%, 40%, 50%, 60%, 70%, 80%, 85%, 90%, 91%, 92%, 93%, 94%, 95%, 96%, 97%, 98%, 99%, or 100% identical to the amino acid sequence set forth in SEQ ID NO: 38. In certain embodiments, a type V-A Cas protein comprises the amino acid sequence set forth in SEQ ID NO: 38.

MAD2
(SEQ ID NO: 38)
MSSLTKFTNKYSKQLTIKNELIPVGKTLENIKENGLIDGDEQLNE
NYQKAKIIVDDELRDFINKALNNTQIGNWRELADALNKEDEDNIE
KLQDKIRGIIVSKFETEDLESSYSIKKDEKIIDDDNDVEEEELDL
GKKTSSFKYIFKKNLFKLVLPSYLKTINQDKLKIISSEDNESTYE
RGFFENRKNIFTKKPISTSIAYRIVHDNFPKELDNIRCENVWQTE
CPQLIVKADNYLKSKNVIAKDKSLANYFTVGAYDYFLSQNGIDFY
NNIIGGLPAFAGHEKIQGLNEFINQECQKDSELKSKLKNRHAFKM
AVLFKQILSDREKSFVIDEFESDAQVIDAVKNFYAEQCKDNNVIE
NLLNLIKNIAFLSDDELDGIFIEGKYLSSVSQKLYSDWSKLRNDI
EDSANSKQGNKELAKKIKTNKGDVEKAISKYEFSLSELNSIVHDN
TKESDLLSCTLHKVASEKLVKVNEGDWPKHLKNNEEKQKIKEPLD
ALLEIYNTLLIFNCKSENKNGNFYVDYDRCINELSSVVYLYNKTR
NYCTKKPYNTDKFKLNENSPQLGEGESKSKENDCLTLLEKKDDNY
YVGIIRKGAKINFDDTQAIADNIDNCIFKMNYELLKDAKKFIPKC
SIQLKEVKAHEKKSEDDYILSDKEKFASPLVIKKSTELLATAHVK
GKKGNIKKFQKEYSKENPTEYRNSLNEWIAFCKEFLKTYKAATIF
DITTLKKAEEYADIVEFYKDVDNLCYKLEFCPIKTSFIENLIDNG
DLYLERINNKDESSKSTGTKNLHTLYLQAIFDERNLNNPTIMLNG
GAELFYRKESIEQKNRITHKAGSILVNKVCKDGTSLDDKIRNEIY
QYENKFIDTLSDEAKKVLPNVIKKEATHDITKDKRFTSDKFFFHC
PLTINYKEGDTKQFNNEVLSFLRGNPDINIIGIDRGERNLIYVTV
INQKGEILDSVSENTVINKSSKIEQTVDYEEKLAVREKERIEAKR
SWDSISKIATLKEGYLSAIVHEICLLMIKHNAIVVLENLNAGFKR
IRGGLSEKSVYQKFEKMLINKLNYFVSKKESDWNKPSGLLNGLQL
SDQFESFEKLGIQSGFIFYVPAAYTSKIDPTTGFANVLNLSKVRN
VDAIKSFFSNFNEISYSKKEALFKESFDLDSLSKKGFSSFVKESK
SKWNVYTFGERIIKPKNKQGYREDKRINLTFEMKKLLNEYKVSED
LENNLIPNLTSANLKDTFWKELFFIFKTTLQLRNSVINGKEDVLI
SPVKNAKGEFFVSGTHNKTLPQDCDANGAYHIALKGLMILERNNL
VREEKDTKKIMAISNVDWFEYVQKRRGVL

In certain embodiments, a type V-A Cas nucleases comprises Csm1. Csm1 proteins are known in the art and are described in U.S. Pat. No. 9,896,696. Csm1 orthologs can be found in various bacterial and archaeal genomes. For example, in certain embodiments, a Csm1 protein is derived from Smithella sp. SCADC (Sm), Sulfuricurvum sp. (Ss), or Microgenomates (Roizmanbacteria) bacterium (Mb).

In certain embodiments, a type V-A Cas nuclease comprises SmCsm1 or a variant thereof. In certain embodiments, a type V-A Cas protein comprises an amino acid sequence at least 30%, 40%, 50%, 60%, 70%, 80%, 85%, 90%, 91%, 92%, 93%, 94%, 95%, 96%, 97%, 98%, 99%, or 100% identical to the amino acid sequence set forth in SEQ ID NO: 12 of International (PCT) Application Publication No. WO 2021/158918. In certain embodiments, a type V-A Cas protein comprises the amino acid sequence set forth in SEQ ID NO: 12 of International (PCT) Application Publication No. WO 2021/158918.

In certain embodiments, a type V-A Cas nuclease comprises SsCsm1 or a variant thereof. In certain embodiments, a type V-A Cas protein comprises an amino acid sequence at least 30%, 40%, 50%, 60%, 70%, 80%, 85%, 90%, 91%, 92%, 93%, 94%, 95%, 96%, 97%, 98%, 99%, or 100% identical to the amino acid sequence set forth in SEQ ID NO: 13 of International (PCT) Application Publication No. WO 2021/158918. In certain embodiments, a type V-A Cas protein comprises the amino acid sequence set forth in SEQ ID NO: 13 of International (PCT) Application Publication No. WO 2021/158918.

In certain embodiments, a type V-A Cas nuclease comprises MbCsm1 or a variant thereof. In certain embodiments, a type V-A Cas protein comprises an amino acid sequence at least 30%, 40%, 50%, 60%, 70%, 80%, 85%, 90%, 91%, 92%, 93%, 94%, 95%, 96%, 97%, 98%, 99%, or 100% identical to the amino acid sequence set forth in SEQ ID NO: 14 of International (PCT) Application Publication No. WO 2021/158918. In certain embodiments, a type V-A Cas protein comprises the amino acid sequence set forth in SEQ ID NO: 14 of International (PCT) Application Publication No. WO 2021/158918.

In certain embodiments, the type V-A Cas nuclease comprises an ART nuclease or a variant thereof. In general, such nucleases sequences have <60% AA sequence similarity to Cas12a, <60% AA sequence similarity to a positive control nuclease, and >80% query cover. In certain embodiments, the Type V-A nuclease comprises an ART1, ART2, ART3, ART4, ART5, ART6, ART7, ART8, ART9, ART10, ART11, ART12, ART13, ART14, ART15, ART16, ART17, ART18, ART19, ART20, ART21, ART22, ART23, ART24, ART25, ART26, ART27, ART28, ART28, ART30, ART31, ART32, ART33, ART34, ART35, or ART11* (i.e., ART11_L679F, i.e., ART11 wherein leucine (L) at amino acid position 679 is replaced with phenylalanine (F)) nuclease, as shown in Table 1. In certain embodiments, the type V-A Cas protein comprises an amino acid sequence at least 30%, 40%, 50%, 60%, 70%, 80%, 85%, 90%, 91%, 92%, 93%, 94%, 95%, 96%, 97%, 98%, 99%, or 100% identical to the amino acid sequence designated for the individual ART nuclease as shown in Table 1. In certain embodiments, provided is a nucleic acid-guided nuclease comprising a nucleic acid-guided nuclease polypeptide having at least 85% identity to an amino acid sequence represented by SEQ ID NOs: 1-36 or a nucleic acid encoding a nucleic acid-guided nuclease polypeptide comprising at least 85% identity with the polynucleotide represented by SEQ ID NOs: 1-36. In certain embodiments, provided is a nucleic acid-guided nuclease comprising a polypeptide having at least 90% identity to the amino acid sequence represented by SEQ ID NOs: 1-36, wherein the polypeptide does not contain a peptide motif of YLFQIYNKDF (SEQ ID NO: 39). In certain embodiments, provided is a nucleic acid-guided nuclease comprising a nucleic acid encoding a polypeptide having at least 90% identity to nucleic acids represented by SEQ ID NOs: 808-845 wherein an encoded polypeptide does not contain a peptide motif of YLFQIYNKDF (SEQ ID NO: 39). In certain embodiments, provided is a nucleic acid-guided nuclease wherein the polypeptide comprises at least 90% identity with the amino acid sequence represented by SEQ ID NOs: 1-9. In certain embodiments, provided is a nucleic acid-guided nuclease, wherein the polypeptide comprises a polypeptide comprising at least 90% identity with the amino acid sequence represented by SEQ ID NO: 2, 11, or 36.

TABLE 1
ART nucleases

| | SEQ | |
|---|---|---|
| | ID | |
| Name | NO | Amino Acid Sequence |
| ART1 | 1 | METFSGFTNLYPLSKTLRFRLIPVGETLKHFIDSGILEEDQHRAESYVK |
| | | VKAIIDDYHRAYIENSLSGFELPLESTKENSLEEYYLYHNIRNKTEEIQ |
| | | NLSSKVRTNLRKQVVAQLTKNEIFKRIDKKELIQSDLIDFVKNEPDANE |
| | | KIALISEFRNFTVYFKGEHENRRNMYSDEEKSTSIAFRLIHENLPKFID |
| | | NMEVFAKIQNTSISENFDAIQKELCPELVTLCEMEKLGYFNKTLSQKQI |
| | | DAYNTVIGGKTTSEGKKIKGLNEYINLYNQQHKQEKLPKMKLLFKQILS |
| | | DRESASWLPEKFENDSQVVGAIVNEWNTIHDTVLAEGGLKTIIASLGSY |
| | | GLEGIFLKNDLQLTDISQKATGSWGKISSEIKQKIEVMNPQKKKESYET |
| | | YQERIDKIFKSYKSFSLAFINECLRGEYKIEDYFLKLGAVNSSSLQKEN |
| | | HFSHILNTYTDVKEVIGLYSESTDTKLIQDNDSIQKIKQFLDAVKDLQA |
| | | YVKPLLGNGDETGKDERFYGDLIEYWSLLDLITPLYNMVRNYVTQKPYS |
| | | VDKIKINFQNPTLLNGWDLNKETDNTSVILRRDGKYYLAIMNNKSRKVF |
| | | LKYPSGTDRNCYEKMEYKLLPGANKMLPKVFFSKSRINEFMPNERLLSN |
| | | YEKGTHKKSGTCFSLDDCHTLIDFFKKSLDKHEDWKNFGFKESDTSTYE |
| | | DMSGFYKEVENQGYKLSFKPIDATYVDQLVDEGKIFLFQIYNKDESEHS |
| | | KGTPNMHTLYWKMLFDETNLGDVVYKLNGEAEVFFRKASINVSHPTHPA |
| | | NIPIKKKNLKHKDEERILKYDLIKDKRYTVDQFQFHVPITMNFKADGNG |
| | | NINQKAIDYLRSASDTHIIGIDRGERNLLYLVVIDGNGKICEQFSLNEI |
| | | EVEYNGEKYSTNYHDLLNVKENERKQARQSWQSIANIKDLKEGYLSQVI |
| | | HKISELMVKYNAIVVLEDLNAGFMRGRQKVEKQVYQKFEKKLIEKLNYL |
| | | VFKKQSSDLPGGLMHAYQLANKFESENTLGKQSGELFYIPAWNTSKMDP |
| | | VTGFVNLEDVKYESVDKAKSFFSKEDSIRYNVERDMFEWKENYGEFTKK |
| | | AEGTKTDWTVCSYGNRIITERNPDKNSQWDNKEINLTENIKLLFERFGI |
| | | DLSSNLKDEIMQRTEKEFFIELISLFKLVLQMRNSWTGTDIDYLVSPVC |
| | | NENGEFFDSRNVDETLPQNADANGAYNIARKGMILLDKIKKSNGEKKLA |
| | | LSITNREWLSFAQGCCKNG |
| ART2 | 2 | MLSNFTNQYQLSKTIRFELKPVGDILKHIEKSGLIAQDEIRSQEYQEVK |
| | | TIIDKYHKAFIDEALQNVVLSNLEEYEALFFERNRDEKAFEKLQAVERK |
| | | EIVAHFKQHPQYKTLFKKELIKADLKNWQELSDAEKELVSHEDNETTYF |
| | | TGEHENRANMYIDEAKHSSIAYRIIHENIPIFLINKKIFETIKQKAPHL |
| | | AQETQDALLEYLSGAIVEDMFELSYFNHLLSQTHIDLYNQMIGGVKQDS |
| | | LKIQGLNEKINLYRQANGLSKRELPNLKPLHKQILSDRETLSWIPESFE |
| | | SDEELMQGVQAYFESEVLAFECCDGKVNLLEKLPELLHQTQDYDESKVY |
| | | FKNDLALTAASQAIFKDYRIIKEALWEVNKPKKSKDLVADEEKFENKKN |
| | | SYFSIEQIDGALNSAQLSANMMHYFQSESTKVIEQIQLTYNDWKRNSSN |
| | | KELLKAFLDALLSYQRLLKPLNAPNDLEKDVAFYAYFDAYFTSLCGVVK |
| | | LYDKVRNFMIKKPYSLEKFKLNFENSTLLDGWDVNKESDNTAILFRKEG |
| | | LYYLGIMNKKYNKVERNISSSQDEGYQKIDYKLLPGANKMLPKVFESDK |
| | | NKEYFKPNAKLLERYKAGEHKKGDNFDLDECHELIDFEKTSIEKHQDWK |
| | | HFAYQFSPTESYEDISGFYREVEQQGYKISYKNIAASFIDTLVAEGKLY |
| | | FFQIYNKDFSPYSKGTPNMHTLYWRALFDEKNLADVIYKINGQAEIFER |
| | | KKSIEYSQEKLQKGHHHEMLKDKFAYPIIKDRREAFDKFQFHVPITINF |
| | | KAEGNENITPKTFEYIRSNPDNIKVIGIDRGERHLLYLSLIDAEGKIVE |
| | | QFTINQIINSYNGKDHVIDYHAKIDAKEKDRDKARKEWGIVENIKELKE |
| | | GYLSHVIHKIATLIIEHGAVVAMEDLNFGEKRGREKVEKQVYQKFEKAL |
| | | IDKLNYLVDKKKEPHKLGGLLNALQLTSKFQSFEKMGKQNGELFYVPAW |
| | | NTSKIDPVTGFVNLEDTRYASVEKSKAFFTKFQSICYNEAKDYFELVED |
| | | YNDFTEKAKETRSEWTLCTYGERIVSFRNAEKNHQWDSKTIHLTTEFKN |
| | | LEGELHGNDVKEYILEQNSVEFEKSLIYLLKITLQMRNSITGTDIDYLV |
| | | SPVADEAGNFYDSRKADTSLPKDADANGAYNIARKGIMIMHRIQNAEDL |
| | | KKVNLAISNRDWLRNAQGLDK |
| ART3 | 3 | MIDLKQFIGIYPVSKTLRFELRPVGKTQEWIEKNRVLEGDEQKAADYPV |
| | | VKKLIDDYHKVCIHDSLNHVHEDWEPLKDAIEIFQKTKSDEAKKRLEAE |
| | | QAMMRKKIAAAIKDFKHFKELTAATPSDLITSVLPEFSDDGSLKSERGE |
| | | ATYFSGFQENRNNIYSQEAISTGVPYRLVHDNFPKELSDLEVFERIKST |
| | | CPEVINQASAELQPFLEGVMIDDIFSLDFYNSLLTQNGIDFFNQVIGGV |
| | | SEKDKQKYRGINEFSNLYRQQHKEIAASKKAMTMIPLFKQILSDRDTLS |
| | | YIPAQIRTEDELVSSITQFYDHITHFEHDGKTINVLSEIVALLGKLDTY |
| | | DPNGICITARKLTDISQKVYGKWSVIEEKMKEKAIQQYGDISVAKNKKK |
| | | VDAFLSRKAYSLSDLCFDEEISESRYYSELPQTLNAISGYWLQFNEWCK |
| | | SDEKQKFLNNQTGTEVVKSLLDAMMELFHKCSVLVMPEEYEVDKSFYNE |
| | | FLPLYEELDTLFLLYNKVRNYLTQKPSDVKKEKLNFESPSLASGWDQNK |
| | | EMKNNAILLFKDGKSYLGVLNAKNKAKIKDAKGDVSSSSYKKMIYKLLS |
| | | DPSKDLPHKIFAKGNLDFYKPSEYILEGRELGKYKKGPNEDKKELHDFI |
| | | DFYKAAISIDPDWSKENFQYSPTESYDDIGMFFSEIKKQAYKIRFTDIS |
| | | EAQVNEWVDNGQLYLFQLYNKDYAEGAHGRKNLHTLYWENLFTDENLSN |
| | | LVLKLNGQAELFCRPQSIKKPVSHKIGSKMLNRRDKSGMPIPESIYRSL |
| | | YQYYNGKKKESELTVAEKQYIDQVIVKDVTHEIIKDRRYTRQEYFFHVP |
| | | LTFNANADGNEYINEHVLNYLKDNPDVNIIGIDRGERHLIYLTLINQRG |
| | | EILKQKTFNVVNSYNYQAKLEQREKERDEARKSWDSVGKIKDLKEGELS |
| | | AVIHEITNMMIENNAIVVLEDLNFGFKRGREKVERQVYQKFEKMLIDKL |
| | | NYLSFKDREAGEEGGILRGYQMAQKFISFQRLGKQSGELFYIPAAYTSK |
| | | IDPVSGFVNHENESDITNAEKRKDELMKMDRIEMKNGNIEFTFDYRKEK |
| | | TFQTDYQNVWTVSTFGKRIVMRIDEKGYKKMVDYEPTNDIIKAFKNKGI |
| | | LLSEGSDLKALIAEIEANATNAGFYSTLLYAFQKTLQMRNSNAVTEEDY |
| | | ILSPVAKDGHQFCSTDEANKGKDAQGNWVSKLPVDADANGAYHIALKGL |
| | | YLLRNPETKKIENEKWLQEMVEKPYLE |
| ART 4 | 4 | MSYNREKMEEKELGKNQNFQEFIGVSPLQKTLRNELIPTETTKKNIAQL |
| | | DLLTEDEVRAQNREKLKEMMDDYYRDVIDSTLRGELLIDWSYLFSCMRN |
| | | HLSENSKESKRELERTQDSVRSQIHDKFAERADFKDMFGASIITKLLPT |
| | | YIKQNSKYSERYDESVKIMKLYGKFTTSLTDYFETRKNIFSKEKISSAV |
| | | GYRIVEENAEIFLQNQNAYDRICKIAGLDLHGLDNEITAYVDGKTLKEV |
| | | CSDEGFAKVITQGGIDRYNEAIGAVNQYMNLLCQKNKALKPGQFKMKRL |
| | | HKQILCKGTTSFDIPKKFENDKQVYDAVNSFTEIVTKNNDLKRLLNITQ |
| | | NANDYDMNKIYVVADAYSMISQFISKKWNLIEECLLDYYSDNLPGKGNA |
| | | KENKVKKAVKEETYRSVSQLNEVIEKYYVEKTGQSVWKVESYISSLAEM |
| | | IKLELCHEIDNDEKHNLIEDDEKISEIKELLDMYMDVFHIIKVERVNEV |
| | | LNFDETFYSEMDEIYQDMQEIVPLYNHVRNYVTQKPYKQEKYRLYFHTP |
| | | TLANGWSKSKEYDNNAIILVREDKYYLGILNAKKKPSKEIMAGKEDCSE |
| | | HAYAKMNYYLLPGANKMLPKVFLSKKGIQDYHPSSYIVEGYNEKKHIKG |
| | | SKNEDIRFCRDLIDYFKECIKKHPDWNKENFEFSATETYEDISVFYREV |
| | | EKQGYRVEWTYINSEDIQKLEEDGQLFLFQIYNKDFAVGSTGKPNLHTL |
| | | YLKNLESEENLRDIVLKLNGEAEIFFRKSSVQKPVIHKCGSILVNRTYE |
| | | ITESGTTRVQSIPESEYMELYRYENSEKQIELSDEAKKYLDKVQCNKAK |
| | | TDIVKDYRYTMDKFFIHLPITINFKVDKGNNVNAIAQQYIAEQEDLHVI |
| | | GIDRGERNLIYVSVIDMYGRILEQKSENLVEQVSSQGTKRYYDYKEKLQ |
| | | NREEERDKARKSWKTIGKIKELKEGYLSSVIHEIAQMVVKYNAIIAMED |
| | | LNYGFKRGRFKVERQVYQKFETMLISKLNYLADKSQAVDEPGGILRGYQ |
| | | MTYVPDNIKNVGRQCGIIFYVPAAYTSKIDPTTGFINAFKRDVVSTNDA |
| | | KENFLMKEDSIQYDIEKGLFKFSFDYKNFATHKLTLAKTKWDVYTNGTR |
| | | IQNMKVEGHWLSMEVELTTKMKELLDDSHIPYEEGQNILDDLREMKDIT |
| | | TIVNGILEIFWLTVQLRNSRIDNPDYDRIISPVLNNDGEFFDSDEYNSY |
| | | IDAQKAPLPIDADANGAFCIALKGMYTANQIKENWVEGEKLPADCLKIE |
| | | HASWLAFMQGERG |
| ART5 | 5 | MSAVFKIKESTMKDFTHQYSLSKTLRFELKPVGETAERIEDFKNQGLKS |
| | | IVEEDRQRAEDYKKMKRILDDYHKEFIEEVLNDDIFTANEMESAFEVYR |
| | | KYMASKNDDKLKKEITEIFTDLRKKIAKAFENKSKEYCLYKGDESKLIN |
| | | EKKTGKDKGPGKLWYWLKAKADAGVNEFGDGQTFEQAEEALAKENNEST |
| | | YFTGENQNRDNIYTDAEQQTAISYRVINENMTRYEDNCIRYSSIENKYP |
| | | ELVKQLEPLSGKFAPGNYKDYLSQTAIDIYNEAVGHKSDDINAKGINQF |
| | | INEYRQRNSIKGRELPIMSVLYKQILSDINKDLIIDKFENAGELLDAVK |
| | | TLHRELTDKKILLKIKQTLNEFLTEDNSEDIYIKSGTDLTAVSNAIWGE |
| | | WSVIPKALEMYAENITDMNAKAREKWLKREAYHLKTVQEAIEAYLKDNE |
| | | EFETRNISEYFTNFKSGENDLIQVVQSAYAKMESIFGIEDEHKDRRPVT |
| | | ESGEPGEGFRQVELVREYLDSLINVEHFIKPLHMERSGKPIELEDCNSN |
| | | FYDPLNEAYKELDVVFGIYNKVRNYVTQKPYSKDKFKINFQNSTLLDGW |
| | | DVNKESANSSVLLLKNGKYYLGVMKQGASNILNYRPEPSDSKNKINAKK |
| | | QLSEIALAGATDDYYEKMIYKLLPDPAKMLPKVFFSAKNIEFYNPSQEI |
| | | IYIRENGLFKKDAGDKESLKKWIGEMKTSLLKHPEWGSYENFEFEPAED |
| | | YQDISIFYKQVAEQGYSVTEDKIKTSYIEEKVASGELYLFEIYNKDESP |
| | | HSKGRPNLHTMYWKSLFEKENLQNLVTKLNGEAEVFFRQHSIKRNEKVV |
| | | HRANRPIQNKNPLTEKKQSIFEYDLVKDRRFTKDKFFLHCPITLNFKEA |
| | | GPGRENDKVNKYIAGNPDIRIIGIDRGERHLLYYSLIDQSGRIVEQGTL |
| | | NQITSTLNSGGREIPKTTDYRGLLDTKEKERDKARKSWSMIENIKELKS |
| | | GYLSHIVHKLAKLMVKNNAVVVLEDLNFGEKRGREKVEKQVYQKFEKAL |
| | | IEKLNYLVFKDARPAEPGHYLNAYQLTAPLESFKKLGKQSGFIYYVPAW |
| | | NTSKIDPVTGFVNQFYIEKNSMQYLKNFFGKEDSIRENPDKNYFEFGED |
| | | YKNFHNKAAKSKWTICTHGDKRSWYNRKQRKLEIHNVTENLASLLSGKG |
| | | INFADGGSIKDKILSVDDASFFKSLAFNFKLTAQLRHTFEDNGEEIDCI |
| | | ISPVAAADGTFFCSETAKKLNMELPHDADANGAYNIARKGLMVLRQIRE |
| | | SGKPKPISNADWLDFAQQNED |
| ART6 | 6 | MQERKKISHLTHRNSVQKTIRMQLNPVGKTMDYFQAKQILENDEKLKEN |
| | | YQKIKEIADRFYRNLNEDVLSKTGLDKLKDYAEIYYHCNTDAERKRLDE |
| | | CASELRKEIVKNFKNRDEYNKLENKKMIEIVLPQHLKNEDEKEVVASFK |
| | | NFTTYFTGFFTNRKNMYSDGEESTAIAYRCINENLPKHLDNVKAFEKAI |
| | | SKLSKNAIDDLDATYSGLCGTNLYDVFTVDYENELLPQSGITEYNKIIG |
| | | GYTTSDGTKVKGINEYINLYNQQVSKRDKIPNLKILYKQILSESEKVSF |
| | | IPPKFEDDNELLSAVSEFYANDETFDGMPLKKAIDETKLLFGNLDNSSL |
| | | NGIYIQNDRSVINLSNSMFGSWSVIEDLWNKNYDSVNSNSRIKDIQKRE |
| | | DKRKKAYKAEKKLSLSFLQVLISNSENDEIREKSIVNYYKTSLMQLTDN |
| | | LSDKYNEAAPLLNKSYANEKGLKNDDKSISLIKNFLDAIKEIEKFIKPL |
| | | SETNITGEKNDLFYSQFTPLLDNISRIDILYDKVRNYVTQKPFSTDKIK |
| | | LNFGNSQLLNGWDRNKEKDCGAVWLCRDEKYYLAIIDKSNNSILENIDE |
| | | QDCDENDCYEKIIYKLLPGPNKMLPKVFFSEKCKKLLSPSDEILKIRKN |
| | | GTFKKGDKFSLDDCHKLIDFYKESFKKYPNWLIYNFKEKNTNEYNDIRE |
| | | FYNDVASQGYNISKMKIPTSFIDKLVDEGKIYLFQLYNKDESPHSKGTP |
| | | NLHTLYFKMLFDERNLEDVVYKLNGEAEMFYRPASIKYDKPTHPKNTPI |
| | | KNKNTLNDKKTSTFPYDLIKDKRYTKWQFSLHFPITMNFKAPDRAMIND |
| | | DVRNLLKSCNNNFIIGIDRGERNLLYVSVIDSNGAIIYQHSLNIIGNKE |
| | | KGKTYETNYREKLATREKERTEQRRNWKAIESIKELKEGYISQAVHVIC |
| | | QLVVKYDAIIVMEKLTDGFKRGRTKFEKQVYQKFEKMLIDKLNYYVDKK |
| | | LDPDEGGGLLHAYQLTNKLESFDKLGMQSGFIFYVRPDFTSKIDPVTGF |
| | | VNLLYPRYENIDKAKDMISREDDIGYNAGEDFFEFDIDYDKEPKTASDY |
| | | RKRWTICTNGERIEAFRNPAKNNEWSYRTIILAEKFKELEDNNSINYRD |
| | | SDDLKAEILSQTKGKFFEDFFKLLRLTLQMRNSNPETGEDRILSPVKDK |
| | | NGNFYDSSKYDEKSKLPCDADANGAYNIARKGLWIVEQFKKSDNVSTVG |
| | | PVIHNDKWLKFVQENDMANN |
| ART7 | 7 | MNILKENYMKEIKELTGLYSLTKTIGVELKPVGKTQELIEAKKLIEQDD |
| | | QRAEDYKIVKDIIDRYHKDFIDKCLNCVKIKKDDLEKYVSLAENSNRDA |
| | | EDFDKIKTKMRNQITEAFRKNSLFTNLFKKNLIKEYLPAFVSEEEKSVV |
| | | NKFSKFTTYFDAENDNRKNLYSGDAKSGTIAYRLIHENLPMELDNIASF |
| | | NAISGIGVNEYESSIETEFTDTLEGKRLTEFFQIDFENNTLTQKKIGNY |
| | | NYIVGAVNKAVNLYKQQHKTVRVPLLKPLYKMILSDRVTPSWLPERFES |
| | | DEEMLTAIKAAYESLREVLVGDNDESLRNLLLNIEHYDLEHIYIANDSG |
| | | LTSISQKIFGCYDTYTLAIKDQLQRDYPATKKQREAPDLYDERIDKLYK |
| | | KVGSFSIAYLNRLVDAKGHFTINEYYKQLGAYCREEGKEKDDFFKRIDG |
| | | AYCAISHLFFGEHGEIAQSDSDVELIQKLLEAYKGLQRFIKPLLGHGDE |
| | | ADKDNEFDAKLRKVWDELDIITPLYDKVRNWLSRKIYNPEKIKLCFENN |
| | | GKLLSGWVDSRTKSDNGTQYGGYIFRKKNEIGEYDFYLGISADTKLERR |
| | | DAAISYDDGMYERLDYYQLKSKTLLGNSYVGDYGLDSMNLLSAFKNAAV |
| | | KFQFEKEVVPKDKENVPKYLKRLKLDYAGFYQILMNDDKVVDAYKIMKQ |
| | | HILATLTSSIRVPAAIELATQKELGIDELIDEIMNLPSKSFGYFPIVTA |
| | | AIEEANKRENKPLFLFKMSNKDLSYAATASKGLRKGRGTENLHSMYLKA |
| | | LLGMTQSVEDIGSGMVFFRHQTKGLAETTARHKANEFVANKNKLNDKKK |
| | | SIFGYEIVKNKRFTVDKYLFKLSMNLNYSQPNNNKIDVNSKVREIISNG |
| | | GIKNIIGIDRGERNLLYLSLIDLKGNIVMQKSLNILKDDHNAKETDYKG |
| | | LLTEREGENKEARRNWKKIANIKDLKRGYLSQVVHIISKMMVEYNAIVV |
| | | LEDLNPGFIRGRQKIERNVYEQFERMLIDKLNFYVDKHKGANETGGLLH |
| | | ALQLTSEFKNFKKSEHQNGCLFYIPAWNTSKIDPATGFVNLENTKYTNA |
| | | VEAQEFFSKEDEIRYNEEKDWFEFEFDYDKFTQKAHGTRTKWTLCTYGM |
| | | RLRSFKNSAKQYNWDSEVVALTEEFKRILGEAGIDIHENLKDAICNLEG |
| | | KSQKYLEPLMQFMKLLLQLRNSKAGTDEDYILSPVADENGIFYDSRSCG |
| | | DQLPENADANGAYNIARKGLMLIEQIKNAEDLNNVKFDISNKAWINFAQ |
| | | QKPYKNG |
| ART8 | 8 | MAKENIFNELTGKYQLSKTLRLELKPVGNTQQMLKDEDVFEKDRIIREK |
| | | YRETRPHFDRLHREFIEQALKNQKLSDLGKYFQCLAKLQNNKKDKEAQE |
| | | EFKRISQNLRKEVNDLFKIDPLFGEGVFALLKEKYGEKDDAFLREQDGQ |
| | | YVLDENKKKISIFDSWKGFTGYFTKFQETRKNFYKDDGTATAVATRIID |
| | | QNLKRFCENIQIFKSIQKKVDFKEVEDNESVDLEDIFSLGFYSSCELQE |
| | | GIDVYNKILGGEPKTTGEKLRGLNELINRYRQDHKGEKLPFFKMLDKQI |
| | | LSEKEKFIESIEDDEELLKTLKEFYSSAEEKTTVLKELENDFIKNNENY |
| | | DLSEIYISREALNTISHRWVSAATLPEFEKSVYEVMKKDKPSGLSEDKD |
| | | DNSYKFPDFIALSYIKGSFEKLSGEKLWKDGYFRDETRNGDKGFLIGNE |
| | | SLWTQFIKIFEFEFNSLFEAKNTERSVGYYHFKKDFEKIITNDESVNPE |
| | | DKVIIREFADNVLAIYQMAKYFAIEKKRKWMDQYDTGDFYNHPDFGYKT |
| | | KFYDNAYEKIVKARMLLQSYLTKKPESTDKWKLNFECGYLLNGWSSSEN |
| | | TYGSLLFRTGNEYYLGVVNGSALRTEKIKRLTGNITEANSCHKMVYDFQ |
| | | KPDNKNVPRIFIRSKGDKFAPAVSELNLPVDSILEIYDKGLEKTENKNS |
| | | PFFKPSLKKLIDYFKLGFSRHASYKHYQFKWKDSSEYKNISEFYNDTIR |
| | | SCYQIKWEELNFEEVKKLTNSKDLFLFQIYNKDFSEKSTGNKNLHSIYF |
| | | DGLFLDNNINAQDGVILKLSGGGEIFFRPKTDVKKLGSRTDTKGKLVIK |
| | | NKRYSQDKIFLHEPIELNYSNTQESNENKLVRNFLADNPDINIIGVDRG |
| | | EKHLIYYAGIDQKGNTLKDKDDKDVLGSLNEINGVNYYKLLEERAKARE |
| | | KARQDWQNIQGIKDLKMGYISLVVRKLADLIIEYNAILVLEDLNMRFKQ |
| | | IHGGIEKSVYQQLEKALIEKLNFLVNKGEKDPERAGHLLRAYQLTAPES |
| | | TFKDMGKQTGVLFYTQASYTSKTCPQCGFRPNIKLHEDNLENAKKMLEK |
| | | INIVYKDNHFEIGYKVSDETKTEKTSRGNILYGDRQGKDTFVISSKAAI |
| | | RYKWFARNIKNNELNRGESLKEHTEKGVTIQYDITECLKILYEKNGIDH |
| | | SGDITKQSIRSELPAKFYKDLLFYLYLLTNTRSSISGTEIDYINCPDCG |
| | | FHSEKGENGCIFNGDANGAYNIARKGMLILKKINQYKDQHHTMDKMGWG |
| | | DLFIGIEEWDKYTQVVSRS |
| ART 9 | 9 | MKEIKELTGLYSLTKTIGVELKPVGKTQELIEAKKLIEQDDQRAEDYKI |
| | | VKDIIDRYHKDFIDKCLNCVKIKKDDLEKYVSLAENSNRDAEDEDKIKT |
| | | KMRNQITEAFRKNSLFTNLFKKNLIKEYLPAFVSEEEKSVVNKESKFTT |
| | | YFDAENDNRKNLYSGDAKSGTIAYRLIHENLPMELDNIASENAISGIGV |
| | | NEYFSSIETEFTDTLEGKRLTEFFQIDFENNTLTQKKIGNYNYIVGAVN |
| | | KAVNLYKQQHKTVRVPLLKPLYKMILSDRVTPSWLPERFESDEEMLTAI |
| | | KAAYESLREVLVGDNDESLRNLLLNIEHYDLEHIYIANDSGLTSISQKI |
| | | FGCYDTYTLAIKDQLQRDYPATKKQREAPDLYDERIDKLYKKVGSESIA |
| | | YLNRLVDAKGHETINEYYKQLGAYCREEGKEKDDFFKRIDGAYCAISHL |
| | | FFGEHGEIAQSDSDVELIQKLLEAYKGLQRFIKPLLGHGDEADKDNEED |
| | | AKLRKVWDELDIITPLYDKVRNWLSRKIYNPEKIKLCFENNGKLLSGWV |
| | | DSRTKSDNGTQYGGYIFRKKNEIGEYDFYLGISADTKLERRDAAISYDD |
| | | GMYERLDYYQLKSKTLLGNSYVGDYGLDSMNLLSAFKNAAVKFQFEKEV |
| | | VPKDKENVPKYLKRLKLDYAGFYQILMNDDKVVDAYKIMKQHILATLTS |
| | | SIRVPAAIELATQKELGIDELIDEIMNLPSKSEGYFPIVTAAIEEANKR |
| | | ENKPLFLFKMSNKDLSYAATASKGLRKGRGTENLHSMYLKALLGMTQSV |
| | | FDIGSGMVFFRHQTKGLAETTARHKANEFVANKNKLNDKKKSIFGYEIV |
| | | KNKRFTVDKYLFKLSMNLNYSQPNNNKIDVNSKVREIISNGGIKNIIGI |
| | | DRGERNLLYLSLIDLKGNIVMQKSLNILKDDHNAKETDYKGLLTEREGE |
| | | NKEARRNWKKIANIKDLKRGYLSQVVHIISKMMVEYNAIVVLEDLNPGE |
| | | IRGRQKIERNVYEQFERMLIDKLNFYVDKHKGANETGGLLHALQLTSEF |
| | | KNFKKSEHQNGCLFYIPAWNTSKIDPATGFVNLENTKYTNAVEAQEFFS |
| | | KFDEIRYNEEKDWFEFEFDYDKFTQKAHGTRTKWTLCTYGMRLRSEKNS |
| | | AKQYNWDSEVVALTEEFKRILGEAGIDIHENLKDAICNLEGKSQKYLEP |
| | | LMQFMKLLLQLRNSKAGTDEDYILSPVADENGIFYDSRSCGDQLPENAD |
| | | ANGAYNIARKGLMLIEQIKNAEDLNNVKEDISNKAWINFAQQKPYKNG |
| ART10 | 10 | MNFQPFFQKFVHLYPISKTLRFELIPQGATQKFISEKQVLLQDEIRARK |
| | | YPEMKQAIDGYHKDFIQRALSNIDSQVFEQALNTFEDLFLRSQAERATD |
| | | AYKKDFETAQTKLRELIVHSFEKGEFKQEYKSLFDKNLITNLLKPWVEQ |
| | | QNQIGDSNYTYHEDENKFTTYFLGFHENRKNIYSKDPHKTALAYRLIHE |
| | | NLPKFLENNKILLKIQNDHPSLWEQLQTLNQTMPQLEDGWDESQLMQVS |
| | | FFSNTLTQTGIDQYNTIIGGISEGENRQKIQGINELINLYNQKQDKKNR |
| | | VAKLKQLYKQILSDRSTLSFLPEKFVDDTELYHAINMFYLEHLHHQSMI |
| | | NGHSYTLLERVQLLINELANYDLSKVYLAPNQLSTVSHQMEGDEGYIGR |
| | | ALNYYYMQVIQPDYEQLLASAKTTKKIEATEKLKTIFLDTPQSLVVIQA |
| | | AIDEYIQLQPSTKPHTQLTDFIISLLKQYETVADDQSIKVINVESDIEG |
| | | KYSCIKGLVNTKSESKREVLQDEKLATDIKAFMDAVNNVIKLLKPFSLN |
| | | EKLVASVEKDARFYSDFEEIYQSLLIFVPLYNKVRNYITQKPYSTEKFK |
| | | LNFNKPTLLSGWDANKEADNLSILLRKNGNYYLAIMDTAKGANKAFEPK |
| | | TLNQLKVDDTTDCYEKMVYKLLSGPSKMFPKAFKAKNNEGNYYPTPELL |
| | | TSYNNNEHLKNDKNFTLASLHAYIDWCKEYINRNPSWHQENFKESPTQS |
| | | FQDISQFYSEVSSQSYKVHFQTIPSDYIDQLVAEGKLYLFQIYNKDESP |
| | | NAKGKENLHTLYFKALFSDENLKQPVFKLSGEAEMFYRPASLQLANTTI |
| | | HKAGEPMAAKNPLTPNATRTLAYDIIKDRRFTTDKYLLHVPISLNFHAQ |
| | | ESMSIKKHNDLVRQMIKHNHQDLHVIGIDRGEKHLLYVSVIDLKGNIVY |
| | | QESLNSIKSEAQNFETPYHQLLQHREEGRAQARTAWGKIENIKELKDGY |
| | | LSQVVHRIQQLILKYNAIVMLEDLNFGEKRGRFKIEKQIYQKFEKALIH |
| | | KLNYVVDKSTQADELGGVRKAYQLTAPFESFEKLGKQSGVLFYVPAWNT |
| | | SKIDPVTGFVDLLKPKYENLDKAQAFFNAFDSIHYNAQKNYFEFKVNLK |
| | | QFAGLKAQAAQAEWTICSYGDERHVYQKKNAQQGETVIVNVTEELKVLF |
| | | AKNNIEVAQSVELKETICTQTQVDFFKRLMWLLQVLLALRYSSSKDKLD |
| | | YILSPVANAQGEFFDSRHASVQLPQDSDANGAYHIALKGLWVIEQLKAA |
| | | DNLDKVKLAISNDDWLHFAQQKPYLA |
| ART11 | 11 | MYYQGLTKLYPISKTIRNELIPVGKTLEHIRMNNILEADIQRKSDYERV |
| | | KKLMDDYHKQLINESLQDVHLSYVEEAADLYLNASKDKDIVDKESKCQD |
| | | KLRKEIVNLLKSHENFPKIGNKEIIKLLQSLSDTEKDYNALDSFSKFYT |
| | | YFTSYNEVRKNLYSDEEKSSTAAYRLINENLPKELDNIKAYSIAKSAGV |
| | | RAKELTEEEQDCLFMTETFERTLTQDGIDNYNELIGKLNFAINLYNQQN |
| | | NKLKGFRKVPKMKELYKQILSEREASFVDEFVDDEALLTNVESESAHIK |
| | | EFLESDSLSRFAEVLEESGGEMVYIKNDTSKTTFSNIVEGSWNVIDERL |
| | | AEEYDSANSKKKKDEKYYDKRHKELKKNKSYSVEKIVSLSTETEDVIGK |
| | | YIEKLQADIIAIKETREVFEKVVLKEHDKNKSLRKNTKAIEAIKSELDT |
| | | IKDFERDIKLISGSEHEMEKNLAVYAEQENILSSIRNVDSLYNMSRNYL |
| | | TQKPFSTEKEKLNENRATLLNGWDKNKETDNLGILLVKEGKYYLGIMNT |
| | | KANKSFVNPPKPKTDNVYHKVNYKLLPGPNKMLPKVFFAKSNLEYYKPS |
| | | EDLLAKYQAGTHKKGENFSLEDCHSLISFFKDSLEKHPDWSEFGFKESD |
| | | TKKYDDLSGFYREVEKQGYKITYTDIDVEYIDSLVEKDELYLFQIYNKD |
| | | FSPYSKGNYNLHTLYLTMLFDERNLRNVVYKLNGEAEVFYRPASIGKDE |
| | | LIIHKSGEEIKNKNPKRAIDKPTSTFEYDIVKDRRYTKDKEMLHIPVTM |
| | | NFGVDETRRENEVVNDAIRGDDKVRVIGIDRGERNLLYVVVVDSDGTIL |
| | | EQISLNSIINNEYSIETDYHKLLDEKEGDRDRARKNWTTIENIKELKEG |
| | | YLSQVVNVIAKLVLKYDAIICLEDLNFGEKRGRQKVEKQVYQKFEKMLI |
| | | DKLNYLVIDKSRSQENPEEVGHVLNALQLTSKFTSFKELGKQTGIIYYV |
| | | PAYLTSKIDPTTGFANLFYVKYESVEKSKDFENREDSICENKVAGYFEF |
| | | SFDYKNFTDRACGMRSKWKVCTNGERIIKYRNEEKNSSFDDKVIVLTEE |
| | | FKKLFNEYGIAFNDCMDLTDAINAIDDASFERKLTKLFQQTLQMRNSSA |
| | | DGSRDYIISPVENDNGEFFNSEKCDKSKPKDADANGAFNIARKGLWVLE |
| | | QLYNSSSGEKLNLAMTNAEWLEYAQQHTI |
| ART12 | 12 | MAKNFEDFKRLYPLSKTLRFEAKPIGATLDNIVKSGLLEEDEHRAASYV |
| | | KVKKLIDEYHKVFIDRVLDNGCLPLDDKGDNNSLAEYYESYVSKAQDED |
| | | AIKKEKEIQQNLLSIIAKKLTDDKAYANLEGNKLIESYKDKADKTKLID |
| | | SDLIQFINTAESTQLVSMSQDEAKELVKEFWGETTYFEGFFKNRKNMYT |
| | | PEEKSTGIAYRLINENLPKFIDNMEAFKKAIARPEIQANMEELYSNESE |
| | | YLNVESIQEMFLLDYYNMLLTQKQIDVYNAIIGGKTDDEHDVKIKGINE |
| | | YINLYNQQHKDDKLPKLKALFKQILSDRNAISWLPEEENSDQEVLNAIK |
| | | DCYERLAENVLGDKVLKSLLGSLADYSLDGIFIRNDLQLTDISQKMEGN |
| | | WGVIQNAIMQNIKHVAPARKHKESEEDYEKRIAGIFKKADSESISYIND |
| | | CLNEADPNNAYFVENYFATFGAVNTPTMQRENLFALVQNAYTEVAALLH |
| | | SDYPTVKHLAQDKANVSKIKALLDAIKSLQHFVKPLLGKGDESDKDERF |
| | | YGELASLWAELDTVTPLYNMIRNYMTRKPYSQKKIKLNFENPQLLGGWD |
| | | ANKEKDYATIILRRNGLYYLAIMDKDSRKLLGKAMPSDGECYEKMVYKF |
| | | FKDVTTMIPKCSTQLKDVQAYFKVNTDDYVLNSKAFNRPLTITKEVEDL |
| | | NNVLYGKYKKFQKGYLTATGDNVGYTHAVNVWIKFCMDELDSYDSTCIY |
| | | DESSLKPESYLSLDSFYQDVNLLLYKLSFTDVSASFIDQLVEEGKMYLE |
| | | QIYNKDFSEYSKGTPNMHTLYWKALFDERNLADVVYKLNGQAEMFYRKK |
| | | SIENTHPTHPANHPILNKNKDNKKKESLFEYDLIKDRRYTVDKEMFHVP |
| | | ITMNEKSSGSENINQDVKAYLRHADDMHIIGIDRGERHLLYLVVIDLQG |
| | | NIKEQFSLNEIVNDYNGNTYHTNYHDLLDVREDERLKARQSWQTIENIK |
| | | ELKEGYLSQVIHKITQLMVRYHAIVVLEDLSKGFMRSRQKVEKQVYQKE |
| | | EKMLIDKLNYLVDKKTDVSTPGGLLNAYQLTCKSDSSQKLGKQSGFLFY |
| | | IPAWNTSKIDPVTGFVNLLDTHSLNSKEKIKAFFSKEDAIRYNKDKKWE |
| | | EFNLDYDKFGKKAEDTRTKWTLCTRGMRIDTFRNKEKNSQWDNQEVDLT |
| | | TEMKSLLEHYYIDIHGNLKDAISTQTDKAFFTGLLHILKLTLQMRNSIT |
| | | GTETDYLVSPVADENGIFYDSRSCGDQLPENADANGAYNIARKGLMLVE |
| | | QIKDAEDLDNVKEDISNKAWLNFAQQKPYKNG |
| ART13 | 13 | MAKNFEDFKRLYSLSKTLRFEAKPIGATLDNIVKSGLLDEDEHRAASYV |
| | | KVKKLIDEYHKVFIDRVLDDGCLPLENKGNNNSLAEYYESYVSRAQDED |
| | | AKKKFKEIQQNLRSVIAKKLTEDKAYANLEGNKLIESYKDKEDKKKIID |
| | | SDLIQFINTAESTQLDSMSQDEAKELVKEFWGFVTYFYGFFDNRKNMYT |
| | | AEEKSTGIAYRLVNENLPKFIDNIEAFNRAITRPEIQENMGVLYSDESE |
| | | YLNVESIQEMFQLDYYNMLLTQKQIDVYNAIIGGKTDDEHDVKIKGINE |
| | | YINLYNQQHKDDKLPKLKALFKQILSDRNAISWLPEEFNSDQEVLNAIK |
| | | DCYERLAENVLGDKVLKSLLGSLADYSLDGIFIRNDLQLTDISQKMFGN |
| | | WGVIQNAIMQNIKRVAPARKHKESEEDYEKRIAGIFKKADSESISYIND |
| | | CLNEADPNNAYFVENYFATFGAVNTPTMQRENLFALVQNAYTEVAALLH |
| | | SDYPTVKHLAQDKANVSKIKALLDAIKSLQHFVKPLLGKGDESDKDERF |
| | | YGELASLWAELDTVTPLYNMIRNYMTRKPYSQKKIKLNFENPQLLGGWD |
| | | ANKEKDYATIILRRNGLYYLAIMDKDSRKLLGKAMPSDGECYEKMVYKF |
| | | FKDVTTMIPKCSTQLKDVQAYFKVNTDDYVLNSKAFNKPLTITKEVEDL |
| | | NNVLYGKYKKFQKGYLTATGDNVGYTHAVNVWIKFCMDELNSYDSTCIY |
| | | DESSLKPESYLSLDAFYQDANLLLYKLSFARASVSYINQLVEEGKMYLE |
| | | QIYNKDFSEYSKGTPNMHTLYWKALFDERNLADVVYKLNGQAEMFYRKK |
| | | SIENTHPTHPANHPILNKNKDNKKKESLFDYDLIKDRRYTVDKEMFHVP |
| | | ITMNFKSVGSENINQDVKAYLRHADDMHIIGIDRGERHLLYLVVIDLQG |
| | | NIKEQYSLNEIVNEYNGNTYHTNYHDLLDVREEERLKARQSWQTIENIK |
| | | ELKEGYLSQVIHKITQLMVRYHAIVVLEDLSKGEMRSRQKVEKQVYQKE |
| | | EKMLIDKLNYLVDKKTDVSTPGGLLNAYQLTCKSDSSQKLGKQSGELFY |
| | | IPAWNTSKIDPVTGFVNLLDTHSLNSKEKIKAFFSKEDAIRYNKDKKWE |
| | | EFNLDYDKFGKKAEDTRTKWTLCTRGMRIDTERNKEKNSQWDNQEVDLT |
| | | TEMKSLLEHYYIDIHGNLKDAISAQTDKAFFTGLLHILKLTLQMRNSIT |
| | | GTETDYLVSPVADENGIFYDSRSCGNQLPENADANGAYNIARKGLMLIE |
| | | QIKNAEDLNNVKFDISNKAWINFAQQKPYKNG |
| ART14 | 14 | MAKNFEDFKRLYSLSKTLRFEAKPIGATLDNIVKSDLLDEDEHRAASYV |
| | | KVKKLIDEYHKVFIDRVLDDGCLPLENKGNNNSLAEYYESYVSRAQDED |
| | | AKKKFKEIQQNLRSVIAKKLTEDKAYANLFGNKLIESYKDKEDKKKIID |
| | | SDLIQFINTAESTQLDSMSQDEAKELVKEFWGFVTYFYGFFDNRKNMYT |
| | | AEEKSTGIAYRLVNENLPKFIDNIEAFNRAITRPEIQENMGVLYSDESE |
| | | YLNVESIQEMFQLDYYNMLLTQKQIDVYNAIIGGKTDDEHDVKIKGIND |
| | | YINLYNQKHKDDKLPKLKALFKQILSDRNAISWLPEEFNSDQEVLNAIK |
| | | DCYERLSENVLGDKVLKSMLGSLADYSLDGIFIRNDLQLTDISQKMEGN |
| | | WSVIQNAIMQNIKHVAPARKHKESEEEYENRIAGIFKKADSESISYIDA |
| | | CLNETDPNNAYFVENYFATLGAVDTPTMQRENLFALVQNAYTEITALLH |
| | | SDYPTEKNLAQDKANVAKIKALLDAIKSLQHFVKPLLGKGDESDKDERF |
| | | YGELASLWAELDTMTPLYNMIRNYMTRKPYSQKKIKLNFENPQLLGGWD |
| | | ANKEKDYATIILRRNGLYYLAIMNKDSKKLLGKAMPSDGECYEKMVYKL |
| | | LPGANKMLPKVFFAKSRMEDFKPSKELVEKYYNGTHKKGKNFNIQDCHN |
| | | LIDYFKQSIDKHEDWSKFGFKESDTSTYEDLSGFYREVEQQGYKLSFAR |
| | | VSVSYINQLVEEGKMYLFQIYNKDFSEYSKGTPNMHTLYWKALFDERNL |
| | | ADVVYKLNGQAEMFYRKKSIENTHPTHPANHPILNKNKDNKKKESLEGY |
| | | DLIKDRRYTVDKFLFHVPITMNFKSSGSENINQDVKAYLRHADDMHIIG |
| | | IDRGERHLLYLVVIDLQGNIKEQFSLNEIVNDYNGNTYHTNYHDLLDVR |
| | | EDERLKARQSWQTIENIKELKEGYLSQVIHKITQLMVKYHAIVVLEDLN |
| | | MGFMRGRQKVEKQVYQKFEKMLIEKLNYLVDKKADASVSGGLLNAYQLT |
| | | SKEDSFQKLGKQSGFLFYIPAWNTSKIDPVTGFVNLLDTRYQNVEKAKS |
| | | FFSKFDAIRYNKDKEWFEFNLDYDKFGKKAEGTRTKWTLCTRGMRIDTE |
| | | RNKEKNSQWDNQEVDLTAEMKSLLEHYYIDIHSNLKDAISAQTDKAFFT |
| | | GLLHILKLTLQMRNSITGTETDYLVSPVVDENGIFYDSRSCGDELPENA |
| | | DANGAYNIARKGLMMIEQIKDAKDLDNLKFDISNKAWLNFAQQKPYKNG |
| ART15 | 15 | MLFQDFTHLYPLSKTVRFELKPIGRTLEHIHAKNELSQDETMADMYQKV |
| | | KVILDDYHRDFIADMMGEVKLTKLAEFYDVYLKFRKNPKDDELQKQLKD |
| | | LQAVLRKESVKPIGNGGKYKAGHDRLFGAKLFKDGKELGDLAKEVIAQE |
| | | GKSSPKLAHLAHFEKESTYFTGFHDNRKNMYSDEDKHTAIAYRLIHENL |
| | | PRFIDNLQILTTIKQKHSALYDQIINELTASGLDVSLASHLDGYHKLLT |
| | | QEGITAYNRIIGEVNGYTNKHNQICHKSERIAKLRPLHKQILSDGMGVS |
| | | FLPSKFADDSEMCQAVNEFYRHYADVFAKVQSLEDGEDDHQKDGIYVEH |
| | | KNLNELSKQAFGDFALLGRVLDGYYVDVVNPEFNERFAKAKTDNAKAKL |
| | | TKEKDKFIKGVHSLASLEQAIKHHTARHDDESVQAGKLGQYFKHGLAGV |
| | | DNPIQKIHNNHSTIKGFLERERPAGERALPKIKSGKNPEMTQLRQLKEL |
| | | LDNALNVAHFAKLLMTKTTLDNQDGNFYGEFGVLYDELAKIPTLYNKVR |
| | | DYLSQKPFSTEKYKLNFGNPTLLNGWDLNKEKDNFGVILQKDGCYYLAL |
| | | LDKAHKKVFDNAPNTGKNVYQKMIYKLLPGPNKMLPRVEFAKSNLDYYN |
| | | PSAELLDKYAQGTHKKGDNFNLKDCHALIDFFKAGINKHPEWQNFGFKF |
| | | SPTSSYRDLSDFYREVEPQGYQVKFVDINADYIDELVEQGQLYLFQIYN |
| | | KDESPKAHGKPNLHTLYFRALESEDNLANPIYKLNGEAQIFYRKASLGM |
| | | NETTIHRAGEILENKNPDNPKERVFTYDIIKDRRYTQDKEMLHVPITMN |
| | | FGVQGMTIKEFNKKVNQSIRQYDDVNVIGIDRGERHLLYLTVINSKGEI |
| | | LEQRSLNDITTASANGTQMTTPYHKILDKREIERLNARVGWGEIETIKE |
| | | LKSGYLSHVVHQVSQLMLKYNAIVVLEDLNFGEKRGREKVEKQIYQNFE |
| | | NALIKKLNHLELKDKADDEIGSYKNALQLTNNFTDLKNIGKQTGELFYV |
| | | PAWNTSKIDPETGFVDLLKPRYENIAQSQAFFGKEDKICYNADKDYFEF |
| | | HIDYAKFTDKAKNSRQTWTICSHGDKRYVYDKTANQNKGATKGINVNDE |
| | | LKSLFARYHINEKQPNLVMDICQNNDKEFHKSLMYLLKTLLALRYSNAS |
| | | SDEDFILSPVANDEGVFENSALADDTQPQNADANGAYHIALKGLWLLNE |
| | | LKNSDDLNKVKLAIDNQTWLNFAQNR |
| ART16 | 16 | MLFQDFTHLYPLSKTVRFELKPIGKTLEHIHAKNFLSQDETMADMYQKV |
| | | KAILDDYHRDFITKMMSEVTLTKLPEFYEVYLALRKNPKDDTLQKQLTE |
| | | IQTALREEVVKPIDSGGKYKAGYERLFGAKLFKDGKELGDLAKEVIAQE |
| | | GESSPKLPQIAHFEKESTYFTGFHDNRKNMYSSDDKHTAIAYRLIHENL |
| | | PRFIDNLQILVTIKQKHSVLYDQIVNELNANGLDVSLASHLDGYHKLLT |
| | | QEGITAYNRIIGEVNSYTNKHNQICHKSERIAKLRPLHKQILSDGMGVS |
| | | FLPSKFADDSEMCQAVNEFYRHYAHVFAKVQSLEDREDDYQKDGIYVEH |
| | | KNLNELSKQAFGDFALLGRVLDGYYVDVVNPEENDKFAKAKTDNAKEKL |
| | | TKEKDKFIKGVHSLASLEQAIEHYIAGHDDESVQAGKLGQYFKHGLAGV |
| | | DNPIQKIHNSHSTIKGFLERERPAGERTLPKIKSDKSLEMTQLRQLKEL |
| | | LDNALNVVHFAKLLTTKTTLDNQDGNFYGEFGALYDELAKIATLYNKVR |
| | | DYLSQKPFSTEKYKLNFGNPTLLNGWDLNKEKDNFGVILQKDGCYYLAL |
| | | LDKAHKKVEDNAPNTGKSVYQKMVYKLLPGPNKMLPKVFFAKSNLDYYN |
| | | PSAELLDKYAQGTHKKGDNENLKDCHALIDFFKASINKHPEWQHFGFEF |
| | | SLTSSYQDLSDFYREVEPQGYQVKFVDIDADYIDELVEQGQLYLFQIYN |
| | | KDFSPKAHGKPNLHTLYFKALFSEDNLANPIYKLNGEAEIFYRKASLDM |
| | | NETTIHRAGEVLENKNPDNPKERQFVYDIIKDKRYTQDKEMLHVPITMN |
| | | FGVQGMTIKEFNKKVNQSIQQYDEVNVIGIDRGERHLLYLTVINSKGEI |
| | | LEQRSLNDIITTSANGTQMTTPYHKILDKREIERLNARVGWGEIETIKE |
| | | LKSGYLSHVVHQISQLMLKYNAIVVLEDLNFGEKRGREKVEKQIYQNFE |
| | | NALIKKLNHLVLKDKADNEIGSYKNALQLTNNFTDLKSIGKQTGFLFYV |
| | | PAWNTSKIDPVTGFVDLLKPRYENIAQSQAFEDKEDKICYNADKGYFEF |
| | | HIDYAKFTDKAKNSRQIWTICSHGDKRYVYDKTANQNKGATIGINVNDE |
| | | LKSLFARYRINDKQPNLVMDICQNNDKEFHKSLTYLLKALLALRYSNAS |
| | | SDEDFILSPVANDKGVFFNSALADDTQPQNADANGAYHIALKGLWLLNE |
| | | LKNSDDLDKVKLAIDNQTWLNFAQNR |
| ART17 | 17 | MLFQDFTHLYPLSKTVRFELKPIGKTLEHIHAKNELSQDETMADMYQKV |
| | | KAILDDYHRDFITKMMSEVTLTKLPEFYEVYLALRKNPKDDTLQKQLTE |
| | | IQTALREEVVKPIDSGGKYKAGYERLFGAKLFKDGKELGDLAKFVIAQE |
| | | GESSPKLPQIAHFEKESTYFTGFHDNRKNMYSSDDKHTAIAYRLIHENL |
| | | PRFIDNLQILVTIKQKHSVLYDQIVNELNANGLDVSLASHLDGYHKLLT |
| | | QEGITAYNRIIGEVNSYTNKHNQICHKSERIAKLRPLHKQILSDGMGVS |
| | | FLPSKFADDSEMCQAVNEFYRHYAHVFAKVQSLFDREDDYQKDGIYVEH |
| | | KNLNELSKQAFGDFALLGRVLDGYYVDVVNPEENDKFAKAKTDNAKEKL |
| | | TKEKDKFIKGVHSLASLEQAIEHYIAGHDDESVQAGKLGQYFKHGLAGV |
| | | DNPIQKIHNSHSTIKGFLERERPAGERTLPKIKSDKSLEMTQLRQLKEL |
| | | LDNALNVVHFAKLLTTKTTLDNQDGNFYGEFGALYDELAKIATLYNKVR |
| | | DYLSQKPFSTEKYKLNFGNPTLLNGWDLNKEKDNFGVILQKDGCYYLAL |
| | | LDKAHKKVEDNAPNTGKSVYQKMVYKLLPGSNKMLPKVFFAKSNLDYYN |
| | | PSAELLDKYAQGTHKKGDNENLKDCHALIDFFKASINKHPEWQHEGFEF |
| | | SLTSSYQDLSDFYREVEPQGYQVKFVDIDADYIDELVEQGQLYLFQIYN |
| | | KDESPKAHGKPNLHTLYFKALFSEDNLANPIYKLNGEAEIFYRKASLDM |
| | | NETTIHRAGEVLENKNPDNPKERQFVYDIIKDKRYTQDKEMLHVPITMN |
| | | FGVQGMTIKEFNKKVNQSIQQYDEVNVIGIDRGERHLLYLTVINSKGEI |
| | | LEQRSLNDIITTSANGTQMTTPYHKILDKREIERLNARVGWGEIETIKE |
| | | LKSGYLSHVVHQISQLMLKYNAIVVLEDLNFGFKRGREKVEKQIYQNFE |
| | | NALIKKLNHLVLKDKADNEIGSYKNALQLTNNFTDLKSIGKQTGELFYV |
| | | PAWNTSKIDPVTGFVDLLKPRYENIAQSQAFEDKEDKICYNADKGYFEF |
| | | HIDYAKFTDKAKNSRQIWTICSHGDKRYVYDKTANQNKGATIGINVNDE |
| | | LKSLFARYRINDKQPNLVMDICQNNDKEFHKSLTYLLKALLALRYSNAS |
| | | SDEDFILSPVANDKGVFFNSALADDTQPQNADANGAYHIALKGLWLLNE |
| | | LKNSDDLDKVKLAIDNQTWLNFAQNR |
| ART18 | 18 | MKYTDFTGIYPVSKTLRFELIPQGSTVENMKREGILNNDMHRADSYKEM |
| | | KKLIDEYHKVFIERCLSDESLKYDDTGKHDSLEEYFFYYEQKRNDKTKK |
| | | IFEDIQVALRKQISKRFTGDTAFKRLEKKELIKEDLPSFVKNDPVKTEL |
| | | IKEFSDFTTYFQEFHKNRKNMYTSDAKSTAIAYRIINENLPKFIDNINA |
| | | FHIVAKVPEMQEHFKTIADELRSHLQVGDDIDKMENLQFENKVLTQSQL |
| | | AVYNAVIGGKSEGNKKIQGINEYVNLYNQQHKKARLPMLKLLYKQILSD |
| | | RVAISWLQDEFDNDQDMLDTIEAFYNKLDSNETGVLGEGKLKQILMGLD |
| | | GYNLDGVFLRNDLQLSEVSQRLCGGWNIIKDAMISDLKRSVQKKKKETG |
| | | ADFEERVSKLFSAQNSFSIAYINQCLGQAGIRCKIQDYFACLGAKEGEN |
| | | EAETTPDIFDQIAEAYHGAAPILNARPSSHNLAQDIEKVKAIKALLDAL |
| | | KRLQRFVKPLLGRGDEGDKDSFFYGDEMPIWEVLDQLTPLYNKVRNRMT |
| | | RKPYSQEKIKLNFENSTLLNGWDLNKEHDNTSVILRREGLYYLGIMNKN |
| | | YNKIFDANNVETIGDCYEKMIYKLLPGPNKMLPKVFFSKSRVQEFSPSK |
| | | KILEIWESKSFKKGDNENLDDCHALIDFYKDSIAKHPDWNKENEKESDT |
| | | QSYTNISDFYRDVNQQGYSLSFTKVSVDYVNRMVDEGKLYLFQIYNKDE |
| | | SPQSKGTPNMHTLYWRMLFDERNLHNVIYKLNGEAEVFYRKASLRCDRP |
| | | THPAHQPITCKNENDSKRVCVEDYDIIKNRRYTVDKEMFHVPITINYKC |
| | | TGSDNINQQVCDYLRSAGDDTHIIGIDRGERNLLYLVIIDQHGTIKEQF |
| | | SLNEIVNEYKGNTYCTNYHTLLEEKEAGNKKARQDWQTIESIKELKEGY |
| | | LSQVIHKISMLMQRYHAIVVLEDLNGSFMRSRQKVEKQVYQKFEHMLIN |
| | | KLNYLVNKQYDAAEPGGLLHALQLTSRMDSFKKLGKQSGELFYIPAWNT |
| | | SKIDPVTGFVNLEDTRYCNEAKAKEFFEKEDDISYNDERDWFEFSFDYR |
| | | HFTNKPTGTRTQWTLCTQGTRVRTERNPEKSNHWDNEEFDLTQAFKDLE |
| | | NKYGIDIASGLKARIVNGQLTKETSAVKDFYESLLKLLKLTLQMRNSVT |
| | | GTDIDYLVSPVADKDGIFFDSRTCGSLLPANADANGAFNIARKGLMLLR |
| | | QIQQSSIDAEKIQLAPIKNEDWLEFAQEKPYL |
| ART19 | 19 | METFSGFTNLYPLSKTLRERLIPVGETLKYFIGSGILEEDQHRAESYVK |
| | | VKAIIDDYHRAYIENSLSGFELPLESTGKENSLEEYYLYHNIRNKTEEI |
| | | QNLSSKVRTNLRKQVVAQLTKNEIFKRIDKKELIQSDLIDFVKNEPDAN |
| | | EKIALISEFRNFTVYFKGFHENRRNMYSDEEKSTSIAFRLIHENLPKFI |
| | | DNMEVFAKIQNTSISENFDAIQKELCPELVTLCEMEKLGYENKTLSQKQ |
| | | IDAYNTVIGGKTTSEGKKIKGLNEYINLYNQQHKQEKLPKMKLLFKQIL |
| | | SDRESASWLPEKFENDSQVVGAIVNEWNTIHDTVLAEGGLKTIIASLGS |
| | | YGLEGIFLKNDLQLTDISQKATGSWGKISSEIKQKIEVMNPQKKKESYE |
| | | TYQERIDKIFKSYKSFSLAFINECLRGEYKIEDYFLKLGAVNSSSLQKE |
| | | NHFSHILNTYTDVKEVIGFYSESTDTKLIRDNGSIQKIKLFLDAVKDLQ |
| | | AYVKPLLGNGDETGKDERFYGDLIEYWSLLDLITPLYNMVRNYVTQKPY |
| | | SVDKIKINFQNPTLLNGWDLNKETDNTSVILRRDGKYYLAIMNNKSRKV |
| | | FLKYPSGTDRNCYEKMEYKLLPGANKMLPKVFFSKSRINEFMPNERLLS |
| | | NYEKGTHKKSGTCFSLDDCHTLIDFFKKSLDKHEDWKNFGFKESDTSTY |
| | | EDMSGFYKEVENQGYKLSFKPIDATYVDQLVDEGKIFLFQIYNKDESEH |
| | | SKGTPNMHTLYWKMLFDETNLGDVVYKLNGEAEVFFRKASINVSHPTHP |
| | | ANIPIKKKNLKHKDEERILKYDLIKDKRYTVDQFQFHVPITMNEKADGN |
| | | GNINQKAIDYLRSASDTHIIGIDRGERNLLYLVVIDGNGKICEQFSLNE |
| | | IEVEYNGEKYSTNYHDLLNVKENERKQARQSWQSIANIKDLKEGYLSQV |
| | | IHKISELMVKYNAIVVLEDLNAGEMRGRQKVEKQVYQKFEKKLIEKLNY |
| | | LVFKKQSSDLPGGLMHAYQLANKFESENTLGKQSGELFYIPAWNTSKMD |
| | | PVTGFVNLEDVKYESVDKAKSFFSKEDSIRYNVERDMFEWKENYGEFTK |
| | | KAEGTKTDWTVCSYGNRIITFRNPDKNSQWDNKEINLTENIKLLFERFG |
| | | IDLSSNLKDEIMQRTEKEFFIELISLEKLVLQMRNSWTGTDIDYLVSPV |
| | | CNENGEFFDSRNVDETLPQNADANGAYNIARKGMILLDKIKKSNGEKKL |
| | | ALSITNREWLSFAQGCCKNG |
| ART20 | 20 | METFSGFTNLYPLSKTLRFRLIPVGETLKHFIDSGILEEDQHRAESYVK |
| | | VKAIIDDYHRAYIENSLSGFELPLESTGKENSLEEYYLYHNIRNKTEEI |
| | | QNLSSKVRTNLRKQVVVQLTKNEIFKRIDKKELIQSDLIDEVKNEPDAN |
| | | EKIALISEFRNFTVYFKGFHENRRNMYSDEEKSTSIAFRLIHENLPKFI |
| | | DNMEVFAKIQNTSISENFDAIQKELCPELVTLCEMEKLGYENKTLSQKQ |
| | | IDAYNTVIGGKTTSEGKKIKGLNEYINLYNQQHKQEKLPKMKLLFKQIL |
| | | SDRESASWLPEKFENDSQVVGAMVNEWNTIHDTVLAEGGLKTIIASLGS |
| | | YGLEGIFLKNDLQLTDISQKATGSWSKISSEIKQKIEVMNPQKKKESYE |
| | | SYQERIDKLFKSYKSFSLAFINECLRGEYKIEDYFLKLGAVNSSSLQKE |
| | | NHFSHILNAYTDVKEAIGFYSESTDTKLIQDNDSIQKIKQFLDAVKDLQ |
| | | AYVKPLLGNGDETGKDERFYGDLIEYWSLLDLITPLYNMVRNYVTQKPY |
| | | SVDKIKINFQNPTLLNGWDLNKETDNTSVILRRDGKYYLAIMNNKSRKV |
| | | FLKYPSGTDGNCYEKMEYKLLPGANKMLPKVFFSKSRINEFMPNERLLS |
| | | NYEKGTHKKSGICFSLDDCHTLIDFFKKSLDKHEDWKNFGFKESDTSTY |
| | | EDMSGFYKEVENQGYKLSFKPIDATYVDQLVDEGKIFLFQIYNKDESEH |
| | | SKGTPNMHTLYWKMLFDETNLGDVVYKLNGEAEVFFRKASINVSHPTHP |
| | | ANIPIKKKNLKHKDEERILKYDLIKDKRYTVDQFQFHVPITMNEKADGN |
| | | GNINQKAIDYLCSASDTHIIGIDRGERNLLYLVVIDGNGKICEQFSLNE |
| | | IEVEYNGEKYSTNYHDLLNVKENERKQARQSWQSIANIKDLKEGYLSQV |
| | | IHKISELMVKYNAIVVLEDLNAGEMRGRQKVEKQVYQKFEKKLIEKLNY |
| | | LVFKKQSSDLPGGLMHAYQLANKFESENALGKQSGELFYIPAWNTSKMD |
| | | PVTGFVNLEDVKYESVDKAKSFFSKEDSMRYNVERDMFEWKENYGEFTK |
| | | KAEGTKTDWTVCSYGNRIITFRNPDKNSQWDNKEINLTENIKLLFERFG |
| | | IDLSSNLKDEIMQRTEKEFFIELISLFKLVLQMRNSWTGTDIDYLVSPV |
| | | CNENGEFFDSRNVDETLPQNADANGAYNIARKGMILLDKIKKSNGEKKL |
| | | ALSITNREWLSFAQGCCKNG |
| ART21 | 21 | METFSGFTNLYPLSKTLRERLIPVGETLKHFIGSGILEEDQHRAESYVK |
| | | VKAIIDDYHRTYIENSLSGFELPLESTGKENSLEEYYLYHNIRNKTEEI |
| | | QNLSSKVRTNLRKQVVTQLTKNEIFKRIDKKELIQSDLIDFVKNEPDAN |
| | | EKIALISEFRNFTVYFKGEHENRRNMYSDEEKSTSIAFRLIHENLPKFI |
| | | DNMEVFAKIQNTSISENFDAIQKELCPELVTLCEMEKLGYENKTLSQKQ |
| | | IDAYNTVIGGKTTSEGKKIKGLNEYINLYNQQHKQEKLPKMKLLFKQIL |
| | | SDRESASWLLEKFENDSQVVGAMVNEWNTIHDTVLAEGGLKTIIASLGS |
| | | YGLEGIFLKNDLQLTDISQKATGSWSKISSEIKQKIEAMNPQKKKESYE |
| | | SYQERIDKLFKSYKSFSLAFVNECLRGEYKIEDYFLKLGAVNSSLLQKE |
| | | NHESHILNTYTDVKEVIGFYSESTDTKLIQDNDSIQKIKQFLDAVKDLQ |
| | | AYVKPLLGNSDETGKDERFYGDLIEYWSLLDLITPLYNMVRNYVTQKPY |
| | | SVDKIKINFQNPTLLNGWDLNKEMDNTSVILRRDGKYYLAIMNNKSRKV |
| | | FLKYPSGTDRNCYEKMEYKLLPGANKMLPKVFFSKSRINEEMPNERLLS |
| | | NYEKGTHKKSGTCFSLDDCHTLIDFFKKSLNKHEDWKNFGFKESDTSTY |
| | | EDMSGFYKEVENQGYKLSFKPIDATYVDQLVDEGKIFLFQIYNKDFSEH |
| | | SKGTPNMHTLYWKMLFDETNLGDVVYKLNGEAEVFFRKASINVSHPTHP |
| | | ANIPIKKKNLKHKDEERILKYDLIKDKRYTVDQFQFHVPITMNEKANGN |
| | | GNINQKAIDYLRSASDTHIIGIDRGERNLLYLVVIDGNGKICEQFSLNE |
| | | IEVEYNGEKYSTNYHDLLNVKENERKQARQSWQSIANIKDLKEGYLSQV |
| | | IHKISELMVKYNAIVVLEDLNAGEMRGRQKVEKQVYQKFEKKLIEKLNY |
| | | LVFKKQSSDLPGGLMHAYQLANKFESENTLGKQSGELFYIPAWNTSKMD |
| | | PVTGFVNLEDVKYESVDKAKSFFSKEDSIRYNVERDMFEWKENYDEFTK |
| | | KAEGTKTDWTVCSYGNRIITFRNPDKNSQWDNKEINLTENIKLLFERFG |
| | | IDLSSNLKDEIMERTEKEFFIELISLFKLVLQMRNSWTGTDIDYLVSPV |
| | | CNENGEFFDSRNVDETLPQNADANGAYNIARKGMILLDKIKKNNGEKKL |
| | | TLSITNREWLSFAQGCCKNG |
| ART22 | 22 | MLFQDFTHLYPLSKTVRFELKPIGKTLEHIHAKNELSQDKTMADMYQKV |
| | | KAILDDYHRDFIADMMGEVKLTKLAEFCDVYLKERKNPKDDGLQKQLKD |
| | | LQAVLRKEIVKPIGNGGKYKVGYDRLFGAKLFKDGKELGDLAKEVIAQE |
| | | SESSPKLPQIAHFEKESTYFTGFHDNRKNMYSSDDKHTAIAYRLIHENL |
| | | PRFIDNLQILATIKQKHSALYDQIASELTASGLDVSLASHLGGYHKLLT |
| | | QEGITAYNRIIGEVNSYTNKHNQICHKSERIAKLRPLHKQILSDGMGVS |
| | | FLPSKFADDSEMCQAVNEFYRHYADVFAKVQSLEDREDDYQKDGIYVEH |
| | | KNLNELSKRAFGDFGELKRFLEEYYADVIDPEFNEKFAKTEPDSDEQKK |
| | | LAGEKDKFVKGVHSLASLEQVIEYYTAGYDDESVQADKLGQYFKHRLAG |
| | | VDNPIQKIHNSHSTIKGFLERERPAGERALPKIKSDKSPEMTQLRQLKE |
| | | LLDNALNVVHFAKLVSTETVLDTRSDKFYGEFRPLYVELAKITTLYNKV |
| | | RDYLSQKPFSTEKYKLNFGNPTLLNGWDLNKEKDNFGVILQKDGCYYLA |
| | | LLDKAHKKVFDNAPNTGKSVYQKMVYKQIANARRDLACLLIINGKVVRK |
| | | TKGLDDLREKYLPYDIYKIYQSESYKVLSPNFNHQDLVKYIDYNKILAS |
| | | GYFEYFDFRFKESSEYKSYKEFLDDVDNCGYKISFCNINADYIDELVEQ |
| | | GQLYLFQIYNKDFSPKAHGKPNLHTLYFKALFSEDNLANPIYKLNGEAQ |
| | | IFYRKASLDMNETTIHRAGEVLENKNPDNPKQRQFVYDIIKDKRYTQDK |
| | | FMLHVPITMNFGVQGMTIEGENKKVNQSIQQYDDVNVIGIDRGERHLLY |
| | | LTVINSKGEILEQRSLNDIITTSANGTQMTTPYHKILNKKKEGRLQARK |
| | | DWGEIETIKELKAGYLSHVVHQISQLMLKYNAIVVLEDLNFGFKRGRLK |
| | | VENQVYQNFENALIKKLNHLVLKDKTDDEIGSYKNALQLTNNFTDLKSI |
| | | GKQTGFLFYVPARNTSKIDPETGFVDLLKPRYENITQSQAFFGKEDKIC |
| | | YNTDKGYFEFHIDYAKFTDEAKNSRQTWVICSHGDKRYVYNKTANQNKG |
| | | ATKGINVNDELKSLFACHHINDKQPNLVMDICQNNDKEFHKSLMYLLKA |
| | | LLALRYSNANSDEDFILSPVANDEGVFENSALADDTQPQNADANGAYHI |
| | | ALKGLWVLEQIKNSDDLDKVDLEIKDDEWRNFAQNR |
| ART23 | 23 | MGKNQNFQEFIGVSPLQKTLRNELIPTETTKKNITQLDLLTEDEIRAQN |
| | | REKLKEMMDDYYRDVIDSTLHAGIAVDWSYLFSCMRNHLRENSKESKRE |
| | | LERTQDSIRSQIYNKFAERADFKDMFGASIITKLLPTYIKQNPEYSERY |
| | | DESMEILKLYGKFTTSLTDYFETRKNIFSKEKISSAVGYRIVEENAEIF |
| | | LQNQNAYDRICKIAGLDLHGLDNEITAYVDGKTLKEVCSDEGEAKAITQ |
| | | EGIDRYNEAIGAVNQYMNLLCQKNKALKPGQFKMKRLHKQILCKGTTSF |
| | | DIPKKFENDKQVYDAVNSFTEIVMKNNDLKRLLNITQNVNDYDMNKIYV |
| | | AADAYSTISQFISKKWNLIEECLLDYYSDNLPGKGNAKENKVKKAVKEE |
| | | TYRSVSQLNELIEKYYVEKTGQSVWKVESYISRLAETITLELCHEIEND |
| | | EKHNLIEDDDKISKIKELLDMYMDAFHIIKVERVNEVLNEDETFYSEMD |
| | | EIYQDMQEIVPLYNHVRNYVTQKPYKQEKYRLYENTPTLANGWSKNKEY |
| | | DNNAIILMRDDKYYLGILNAKKKPSKQTMAGKEDCLEHAYAKMNYYLLP |
| | | GANKMLPKVELSKKGIQDYHPSSYIVEGYNEKKHIKGSKNEDIRFCRDL |
| | | IDYFKECIKKHPDWNKENFEFSATETYEDISVFYREVEKQGYRVEWTYI |
| | | NSEDIQKLEEDGQLFLFQIYNKDFAVGSTGKPNLHTLYLKNLESEENLR |
| | | DIVLKLNGEAEIFFRKSSVQKPVIHKCGSILVNRTYEITESGTTRVQSI |
| | | PESEYMELYRYENSEKQIELSDEAKKYLDKVQCNKAKTDIVKDYRYTMD |
| | | KFFIHLPITINFKVDKGNNVNAIAQQYIAEQEDLHVIGIDRGERNLIYV |
| | | SVIDMYGRILEQKSENLVEQVSSQGTKRYYDYKEKLQNREEERDKARKS |
| | | WKTIGKIKELKEGYLSSVIHEIAQMVVKYNAIIAMEDLNYGEKRGREKV |
| | | ERQVYQKFETMLISKLNYLADKSQAVDEPGGILRGYQMTYVPDNIKNVG |
| | | RQCGIIFYVPAAYTSKIDPTTGFINAFKRDVVSTNDAKENELMKEDSIQ |
| | | YDIEKGLFKFSFDYKNFATHKLTLAKTKWDVYINGTRIQNMKVEGHWLS |
| | | MEVELTTKMKELLDDSHIPYEEGQNILDDLREMKDITTIVNGILEIFWL |
| | | TVQLRNSRIDNPDYDRIISPVLNNDGEFFDSDEYNSYIDAQKAPLPIDA |
| | | DANGAFCIALKGMYTANQIKENWVEGEKLPADCLKIEHASWLAFMQGER |
| | | G |
| ART24 | 24 | MNTSLFSSFTRQYPVTKTLRFELKPMGATLGHIQQKGFLHKDEELAKIY |
| | | KKIKELLDEYHRAFIADTLGDAQLVGLDDFYADYQALKQDSKNSHLKDK |
| | | LTKTQDNLRKQITKNFEKTPQLKERYKRLFTKELFKAGKDKGDLEKWLI |
| | | NHDSEPNKAEKISWIHQFENFTTYFQGFYENRKNMYSDEVKHTAIAYRL |
| | | IHENLPRFVDNIQVLSKIKSDYPDLYHELNHLDSRTIDFADEKEDDMLQ |
| | | MDFYHHLLIQSGITAYNTLLGGKVLEGGKKLQGINELINLYGQKHKIKI |
| | | AKLKPLHKQILSDGQSVSFLPKKFDNDYELCQTVNHFYREYVAIFDELV |
| | | VLFQKFYDYDKDNIYINHQQLNQLSHELFADERLLSRALDFYYCQIIDG |
| | | DENNKINNAKSQNAKEKLLKEKERYTKSNHSINELQKAINHYASHHEDT |
| | | EVKVISDYFSATNIRNMIDGIHHHESTIKGFLEKDNNQGESYLPKQKNS |
| | | NDVKNLKLFLDGVLRLIHFIKPLALKSDDTLEKEEHFYGEFMPLYDKLV |
| | | MFTLLYNKVRDYISQKPYNDEKIKLNFGNSTLLNGWDVNKEKDNFGVIL |
| | | CKEGLYYLAILDKSHKKVEDNAPKATSSHTYQKMVYKLLPGPNKMLPKV |
| | | FFAKSNIGYYQPSAQLLENYEKGTHKKGSNFSLTDCHHLIDFFKSSIAK |
| | | HPEWKEFGERESDTHTYQDLSDFYKEIEPQSYKVKFIDIDADYIDDLVE |
| | | KGQLYLFQLYNKDFSKQSYGKPNLHTLYFKSLFSDDNLKNPIYKLNGEA |
| | | EIFYRRASLSVSDTTIHQAGEILTPKNPNNTHNRTLSYDVIKNKRYTTD |
| | | KFFLHIPITMNFGIENTGFKAFNHQVNTTLKNADKKDVHIIGIDRGERH |
| | | LLYVSVIDGDGRIVEQRTLNDIVSISNNGMSMSTPYHQILDNREKERLA |
| | | ARTDWGDIKNIKELKAGYLSHVVHEVVQMMLKYNAMIVLEDLNFGEKHG |
| | | RFKVEKQVYQNFENALIKKLNYLVLKNADNHQLGSVRKALQLTNNFTDI |
| | | KSIGKQTGFIFYVPAWNTSKIDPTTGFVDLLKPRYENMAQAQSFISREK |
| | | KIAYNHQLDYFEFEFDYADFYQKTIDKKRIWTLCTYGDVRYYYDHKTKE |
| | | TKTVNITKELKSLLDKHDLSYQNGHNLVDELANSHDKSLLSGVMYLLKV |
| | | LLALRYSHAQKNEDFILSPVMNKDGVFFDSRFADDVLPNNADANGAYHI |
| | | ALKGLWVLNQIQSADNMDKIDLSISNEQWLHFTQSR |
| ART25 | 25 | MVGNKISNSFDSFTGINALSKTLRNELIPSDYTKRHIAESDFIAADINK |
| | | NEDQYVAKEMMDDYYRDFISKVLDNLHDIEWKNLFELMHKAKIDKSDAT |
| | | SKELIKIQDMLRKKIGKKESQDPEYKVMLSAGMITKILPKYILEKYETD |
| | | REDRLEAIKRFYGFTVYFKEFWASRQNVESDKAIASSISYRIIHENAKI |
| | | YMDNLDAYNRIKQIACEEIEKIEEEAYDFLQGDQLDVVYTEEAYGRFIS |
| | | QSGIDLYNNICGVINAHMNLYCQSKKCSRSKFKMQKLHKQILCKAETGE |
| | | EIPLGFQDDAQVINAINSENALIKEKNIISRLRTIGKSISLYDVNKIYI |
| | | SSKAFENVSVYIDHKWDVIASSLYKYFSEIVKGNKDNREEKIQKEIKKV |
| | | KSCSLGDLQRLVNSYYKIDSTCLEHEVTEFVTKIIDEIDNFQITDEKEN |
| | | DKISLIQNEQIVMDIKTYLDKYMSIYHWMKSFVIDELVDKDMEFYSELD |
| | | ELNEDMSEIVNLYNKVRNYVTQKPYSQEKIKLNFGSPTLADGWSKSKEF |
| | | DNNAIILIRDEKIYLAIFNPRNKPAKTVISGHDVCNSETDYKKMNYYLL |
| | | PGASKTLPHVFIKSRLWNESHGIPDEILRGYELGKHLKSSVNEDVEFCW |
| | | KLIDYYKECISCYPNYKAYNFKFADTESYNDISEFYREVECQGYKIDWT |
| | | YISSEDVEQLDRDGQIYLFQIYNKDFAPNSKGMDNLHTKYLKNIFSEDN |
| | | LKNIVIKLNGEAELFYRKSSVKKKVEHKKGTILVNKTYKVEDNTENSKE |
| | | KRVIIESVPDDCYMELVDYWRNGGIGILSDKAVQYKDKVSHYEATMDIV |
| | | KDRRYTVDKFFIHLPITINFKADGRININEKVLKYIAENDELHVIGIDR |
| | | GERNLLYVSVINKKGKIVEQKSENMIESYETVINIVRRYNYKDKLVNKE |
| | | SARTDARKNWKEIGKIKEIKEGYLSQVIHEISKMVLKYNAIIVMEDLNY |
| | | GFKRGRFRVERQVYQKFENMLISKLAYLVDKSRKADEPGGVLRGYQLTY |
| | | IPDSLEKLGSQCGIIFYVPAAYTSKIDPLTGFVNVENFREYSNFETKLD |
| | | FVRSLDSIRYDTEKKLESISFDYDNFKTHNTTLAKTKWVIYLRGERIKK |
| | | EHTSYGWKDDVWNVESRIKDLEDSSHMKYDDGHNLIEDILELESSVQKK |
| | | LINELIEIIRLTVQLRNSKSERYDRTEAEYDRIVSPVMDENGREYDSEN |
| | | YIFNEETELPKDADANGAYCIALKGLYNVIAIKNNWKEGEKENRKLLSL |
| | | NNYNWEDFIQNRRF |
| ART26 | 26 | MVGNKISNSFDSFTGINALSKTLRNELIPSDYTKRHIAESDFIAADTNK |
| | | NEDQYVAKEMMDDYYRDFISKVLDNLHDIEWKNLFELMHKAKIDKSDAT |
| | | SKELIKIQDMLRKKIGKKESQDPEYKVMLSAGMITKILPKYILEKYETD |
| | | REDRLEAIKRFYGFTVYFKEFWASRQNVESDKAIASSISYRIIHENAKI |
| | | YMDNLDAYNRIKQIACEEIEKIEEEAYDFLQGDQLDVVYTEEAYGRFIS |
| | | QSGIDLYNNICGVINAHMNLYCQSKKCSRSKFKMQKLHKQILCKAETGF |
| | | EIPLGFQDDAQVINAINSENALIKEKNIISRLRTIGKSISLYDVNKIYI |
| | | SSKAFENVSVYIDHKWDVIASSLYKYFSEIVKGNKDNREEKIQKEIKKV |
| | | KSCSLGDLQRLVNSYYKIDSTCLEHEVTEFVTKIIDEIDNFQITDEKEN |
| | | DKISLIQNEQIVMDIKTYLDKYMSIYHWMKSFVIDELVDKDMEFYSELD |
| | | ELNEDMSEIVNLYNKVRNYVTQKPYSQEKIKLNFGSPTLADGWSKSKEF |
| | | DNNAIILIRDEKIYLAIFNPRNKPAKTVISGHDVCNSETDYKKMNYYLL |
| | | PGASKTLPHVFIKSRLWNESHGIPDEILRGYELGKHLKSSVNEDVEFCW |
| | | KLIDYYKECISCYPNYKAYNEKFADTESYNDISEFYREVECQGYKIDWT |
| | | YISSEDVEQLDRDGQIYLFQIYNKDFAPNSKGMDNLHTKYLKNIFSEDN |
| | | LKNIVIKLNGEAELFYRKSSVKKKVEHKKGTILVNKTYKVEDNTENSKE |
| | | KRVIIESVPDDCYMELVDYWRNGGIGILSDKAVQYKDKVSHYEATMDIV |
| | | KDRRYTVDKFFIHLPITINFKADGRININEKVLKYIAENDELHVIGIDR |
| | | GERNLLYVSVINKKGKIVEQKSENMIESYETVTNIVRRYNYKDKLVNKE |
| | | SARTDARKNWKEIGKIKEIKEGYLSQVIHEISKMVLKYNAIIVMEDLNY |
| | | GFKRGRFRVERQVYQKFENMLISKLAYLVDKSRKADEPGGVLRGYQLTY |
| | | IPDSLEKLGSQCGIIFYVPAAYTSKIDPLTGFVNVENFREYSNFETKLD |
| | | FVRSLDSIRYDTEKKLESISFDYDNFKTHNTTLAKTKWVIYLRGERIKK |
| | | EHTSYGWKDDVWNVESRIKDLFDSSHMKYDDGHNLIEDILELESSVQKK |
| | | LINELIEIIRLTVQLRNSKSERYDRTEAEYDRIVSPVMDENGRFYDSEN |
| | | YIFNEETELPKDADANGAYCIALKGLYNVIAIKNNWKEGEKENRKLLSL |
| | | NNYNWFDFIQNRRFQIYLFQIYNKDFAPNSKGMDNLHTKYLKNIFSEDN |
| | | LKNIVIKLNGEAELFYRKSSVKKKVEHKKGTILVNKTYKVEDNTENSKE |
| | | KRVIIESVPDDCYMELVDYWRNGGIGILSDKAVQYKDKVSHYEATMDIV |
| | | KDRRYTVDKFFIHLPITINFKADGRININEKVLKYIAENDELHVIGIDR |
| | | GERNLLYVSVINKKGKIVEQKSENMIESYETVINIVRRYNYKDKLVNKE |
| | | SARTDARKNWKEIGKIKEIKEGYLSQVIHEISKMVLKYNAIIVMEDLNY |
| | | GFKRGRFRVERQVYQKFENMLISKLAYLVDKSRKADEPGGVLRGYQLTY |
| | | IPDSLEKLGSQCGIIFYVPAAYTSKIDPLTGFVNVENFREYSNFETKLD |
| | | FVRSLDSIRYDTEKRLFSISEDYDNEKTHNTTLAKTKWVIYLRGERIKK |
| | | EHTSYGWKDDVWNVESRIKDLFDSSHMKYDDGHNLIEDILELESSVQKK |
| | | LINELIEIIRLTVQLRNSKSERYDRTEAEYDRIVSPVMDEKGRFYDSEN |
| | | YIFNEETELPKDADANGAYCIALKGLYNVIAIKNNWKEGEKENRKLLSL |
| | | NNYNWEDFIQNRRE |
| ART27 | 27 | MQEHKKISHLTHRNSVQKTIRMQLNPVGKTMDYFQAKQILENDEKLKED |
| | | YQKIKEIADRFYRNLNEDVLSKTGLDKLKDYAEIYYHCNTDADRKRLDE |
| | | CASELRKEIVKNFKNRDEYNKLENKKMIEIVLPQHLKNEDEKEVVASFK |
| | | NFTTYFTGFFTNRKNMYSDGEESTAIAYRCINENLPKHLDNVKAFEKAI |
| | | SKLSKNAVDDLDTTYSGLCGTNLYDVFTVDYENELLPQSGITEYNKIIG |
| | | GYTTSDGTKVKGINEYINLYNQQVSKRYKIPNLKILYKQILSESEKVSF |
| | | IPPKFEDDNELLSAVSEFYANDETFDGMPLKKAIDETKLLFGNLDNSSL |
| | | NGIYIQNDRSVTNLSNSMFGSWSVIEDLWNKNYDSVNSNSRIKDIQKRE |
| | | DKRKKAYKAEKKLSLSFLQVLISNSENDEIREKSIVDYYKTSLMQLTDN |
| | | LSDKYKEAAPLENESYANEKGLKNDDKSISLIKNFLDAIKEIEKFIKPL |
| | | SETNITGEKNDLFYSQFTPLLDNISRIDILYDKVRNYVTQKPFSTDKIK |
| | | LNFGNSQLLNGWDRNKEKDCGAVWLCKDEKYYLAIIDKSNNSILENIDE |
| | | QDCDESDCYEKIIYKLLPGPNKMLPKVFFSEKCKKLLSPSDEILKIRKN |
| | | GTFKKGDKESLDDCHKLIDFYKESFKKYPNWLIYNFKFKKTNEYNDISE |
| | | FYNDVASQGYNISKMKIPTSFIDKLVDEGKIYLFQLYNKDESPHSKGTP |
| | | NLHTLYFKMLFDERNLEDVVYKLNGEAEMFYRPASIKYDKPTHPKNTPI |
| | | KNKNTLNDKRASTFPYDLIKDKRYTKWQFSLHEPITMNFKAPDRAMIND |
| | | DVRNLLKSCNNNFIIGIDRGERNLLYVSIIDSNGAIIYQHSLNIIGNKE |
| | | KGKTYETNYREKLETREKERTEQRRNWKAIESIKELKEGYISQAVHVIC |
| | | QLVVKYDAIIVMEKLTDGFKRGRTKFEKQVYQKFEKMLIDKLNYYVDKK |
| | | LDPDEEGGLLHAYQLTNKLESFDKLGMQSGFIFYVRPDFTSKIDPVTGF |
| | | VNLLYPRYENIDKAKDMISREDDIRYNAGEDFFEFDIDYDKFPKTASDY |
| | | RKKWTICTNGERIEAFRNPASNNEWSYRTIILAEKFKELEDNNSINYRD |
| | | SDNLKAEILSQTKGKFFEDFFKLLRLTLQMRNSNPETGEDRILSPVKDK |
| | | NGNFYDSSKYDEKSNLPCDADANGAYNIARKGLWIVEQFKKSDNVSTVE |
| | | PVIHNDKWLKFVQENDMANN |
| ART28 | 28 | MKNLANFTNLYSLQKTLRFELKPIGKTLDWIIKKDLLKQDEILAEDYKI |
| | | VKKIIDRYHKDFIDLAFESAYLQKKSSDSFTAIMEASIQSYSELYFIKE |
| | | KSDRDKKAMEEISGIMRKEIVECFTGKYSEVVKKKFGNLFKKELIKEDL |
| | | LNFCEPDELPIIQKFADETTYFTGEHENRENMYSNEEKATAIANRLIRE |
| | | NLPRYLDNLRIIRSIQGRYKDEGWKDLESNLKRIDKNLQYSDELTENGE |
| | | VYTFSQKGIDRYNLILGGQSVESGEKIQGLNELINLYRQKNQLDRRQLP |
| | | NLKELYKQILSDRTRHSFVPEKESSDKALLRSLLDFHKEVIQNKNLFEE |
| | | KQVSLLQAIRETLTDLKSEDLDRIYLINDTSLTQISNFVFGDWSKVKTI |
| | | LAIYFDENIANPKDRQRQSNSYLKAKENWLKKNYYSIHELNEAISVYGK |
| | | HSDEELPNTKIEDYFSGLQTKDETKKPIDVLDAIVSKYADLESLLTKEY |
| | | PEDKNLKSDKGSIEKIKNYLDSIKLLQNFLKPLKPKKVQDEKDLGFYND |
| | | LELYLESLESANSLYNKVRNYLTGKEYSDEKIKLNFKNSTLLDGWDENK |
| | | ETSNLSVIFRDINNYYLGILDKQNNRIFESIPEIQSGEETIQKMVYKLL |
| | | PGANNMLPKVFFSEKGLLKENPSDEITSLYSEGRFKKGDKFSINSLHTL |
| | | IDFYKKSLAVHEDWSVENFKFDETSHYEDISQFYRQVESQGYKITEKPI |
| | | SKKYIDTLVEDGKLYLFQIYNKDESQNKKGGGKPNLHTIYFKSLFEKEN |
| | | LKDVIVKLNGQAEVFFRKKSIHYDENITRYGHHSELLKGRFSYPILKDK |
| | | RFTEDKFQFHFPITLNFKSGEIKQFNARVNSYLKHNKDVKIIGIDRGER |
| | | HLLYLSLIDQDGKILRQESLNLIKNDQNFKAINYQEKLHKKEIERDQAR |
| | | KSWGSIENIKELKEGYLSQVVHTISKLMVEHNAIVVLEDLNFGEKRGRQ |
| | | KVERQVYQKFEKMLIEKLNFLVEKDKEMDEPGGILKAYQLTDNFVSFEK |
| | | MGKQTGFVFYVPAWNTSKIDPKTGFVNELHLNYENVNQAKELIGKEDQI |
| | | RYNQDRDWFEFQVTTDQFFTKENAPDTRTWIICSTPTKRFYSKRTVNGS |
| | | VSTIEIDVNQKLKELFNDCNYQDGEDLVDRILEKDSKDFFSKLIAYLRI |
| | | LTSLRQNNGEQGFEERDFILSPVVGSDGKFFNSLDASSQEPKDADANGA |
| | | YHIALKGLMNLHVINETDDESLGKPSWKISNKDWLNFVWQRPSLKA |
| ART29 | 29 | MQEHKKISHLTHRNSVQKTIRMQLNPVGKTMDYFQAKQILENDEKLKEN |
| | | YQKIKEIADRFYRNLNEDVLSKTRLDKLKDYTDIYYHCNTDADRKRLDE |
| | | CASELRKEIVKNEKNRDEYNKLENKKMIEIVLPKHLKNEDEKEVVTSEK |
| | | NFTTYFTGFFTNRKNMYSDGEESTAIAYRCINENLPKHLDNVKAFEKAI |
| | | SKLSKNAIDDLDTTYSGLCGTNLYDVFTVDYENELLPQSGITEYNKIIG |
| | | GYTTNDGTKVKGINEYINLYNQQVSKRDKIPNLKILYKQILSESEKVSF |
| | | IPPKFEDDNELLSAVSEFYANDETFDGMPLKKAIDETKLLEGNLDNPSL |
| | | NGIYIQNDRSVINLSNSMFGSWSVIEDLWNKNYDSVNSNSRIKDIQKRE |
| | | DKRKKAYKAEKKLSLSFLQVLISNSENDEIREKSIVDYYKTSLMQLTDN |
| | | LSDKYNEAAPLLNENYSNEKGLKNDDKSISLIKNFLDAIKEIEKFIKPL |
| | | SETNITGEKNDLFYSQFTPLLDNISRIDILYDKVRNYVTQKPFSTDKIK |
| | | LNFGNSQLLNGWDRNKEKDCGAVWLCKDEKYYLAIIDKSNNSILENIDE |
| | | QDCDESDCYEKIIYKLLPGPNKMLPKVFFSEKCKKLLSPSDEILKIYKS |
| | | GTFKTGDKFSLDDCHKLIDFYKESFKKYPNWLIYNEKFKKTNEYNDIRE |
| | | FYNDVALQGYNISKMKIPTSFIDKLVDEGKIYLFQLYNKDESPHSKGTP |
| | | NLHTLYFKMLFDERNLEDVVYRLNGEAEMFYRPASIKYDKPTHPKNTPI |
| | | KNKNTLNDKKTSTFPYDLIKDKRYTKWQFSLHFPITMNFKAPDKAMIND |
| | | DVRNLLKSCNNNFIIGIDRGERNLLYVSVIDSNGAIIYQHSLNIIGNKE |
| | | KEKTYETNYREKLATREKERTEQRRNWKAIESIKELKEGYISQAVHVIC |
| | | QLVVKYDAIIVMEKLTDGFKRGRTKFEKQVYQKFEKMLIDKLNYYVDKK |
| | | LDPDEEGGLLHAYQLTNKLESFDKLGMQSGFIFYVRPDFTSKIDPVTGF |
| | | VNLLYPQYENIDKAKDMISREDEIRYNAGEDFFEFDIDYDEFPKTASDY |
| | | RKKWTICTNGERIEAFRNPANNNEWSYRTIILAEKFKELFDNNSINYRD |
| | | SDDLKAEILSQTKGKFFEDFFKLLRLTLQMRNSNPETGEDRILSPVKDK |
| | | NGNFYDSSKYDEKSKLPCDADANGAYNIARKGLWIVEQFKKADNVSTVE |
| | | PVIHNDQWLKFVQENDMANN |
| ART30 | 30 | MQEHKKISHLTHRNSVQKTIRMQLNPVGKTMDYFQAKQILENDEKLKED |
| | | YQKIKEIADRFYRNLNEDVLSKTGLDKLKDYADIYYHCNTDADRKRLNE |
| | | CASELRKEIVKNEKNRDEYNKLENKKMIEIVLPKHLKNEDEKEVVASEK |
| | | NFTTYFTGFFTNRKNMYSDGEESTAIAYRCINENLPKHLDNVKVFEKAI |
| | | SKLSKNAIDDLGATYSGLCGTNLYDVFTVDYENELLPQSGITEYNKIIG |
| | | GYTTSDGTKVKGINEYINLYNQQVSKRDKIPNLKILYKQILSESEKVSF |
| | | IPPKFEDDNELLSAVSEFYANDETFDGMPLKKAIDETKLLEGNLDNSSL |
| | | NGIYIQNDRSVINLSNSMFGSWSVIEDLWNKNYDSVNSNSRIKDIQKRE |
| | | DKRKKAYKAEKKLSLSFLQVLISNSENDEIREKSIVDYYKTSLMQLTDN |
| | | LSDKYKEAAPLESENYDNEKGLKNDDKSISLIKNFLDAIKEIEKFIKPL |
| | | SETNITGEKNDLFYSQFTPLLDNISRIDILYDKVRNYVTQKPFSTDKIK |
| | | LNFGNSQLLNGWDKDKEREYGAVLLCKDEKYYLAIIDKSNNSILENIDE |
| | | QDCNESDYYEKIVYKLLTKINGNLPRVFFSEKRKKLLSPSDEILKIYKS |
| | | GTFKKGDKFSLDDCHKLIDFYKESFKKYPNWLIYNFKEKNTNEYNDISE |
| | | FYNDVASQGYNISKMKIPTTFIDKLVDEGKIYLFQLYNKDESPHSKGTP |
| | | NLHTLYFKMLFDERNLEDVVYKLNGEAEMFYRPASIKYDKPTHPKNTPI |
| | | KNKNTLNDKKASTFPYDLIKDKRYTKWQFSLHEPITMNFKAPDKAMIND |
| | | DVRNLLKSCNNNFIIGIDRGERNLLYVSVIDSNGAIIYQHSLNIIGNKE |
| | | KGKTYETNYREKLATREKDRTEQRRNWKAIESIKELKEGYISQAVHVIC |
| | | QLVVKYDAIIVMEKLTDGFKRGRTKFEKQVYQKFEKMLIDKLNYYVDKK |
| | | LDPDEEGGLLHAYQLTNKLESFDKLGTQSGFIFYVRPDETSKIDPVTGF |
| | | VNLLYPRYENIDKAKDMISREDDIRYNAGEDFFEFDIDYDKFPKTASDY |
| | | RKKWTICINGERIEAFRNPANNNEWSYRTIILAEKFKELEDNNSINYRD |
| | | SDDLKAEILSQTKGKFFEDFFKLLRLTLQMRNSNPETGEDRILSPVKDK |
| | | NGNFYDSSKYDEKSKLPCDADANGAYNIARKGLWIVEQFKKADNVSTVE |
| | | PVIHNDKWLKFVQENDMANN |
| ART31 | 31 | MQERKKISHLTHRNSVKKTIRMQLNPVGKTMDYFQAKQILENDEKLKEN |
| | | YQKIKEIADRFYRNLNEDVLSKTGLDKLKDYAEIYYHCNTDADRKRLNK |
| | | CASELRKEIVKNEKNRDEYNKLEDKRMIEIVLPKHLKNEDEKEVVASEK |
| | | NETTYFTGFFTNRKNMYSDGEESTAIAYRCINENLPKHLDNVKAFEKAI |
| | | SKLSKNAIDDLDAYSGLCGTNLYDVFTVDYFNELLPQSGITEYNKIIGG |
| | | YTTNDGTKVKGINEYINLYNQQVSKRDKIPNLQILYKQILSESEKVSFI |
| | | PPKFEDDNELLSAVSEFYANDETFDGMPLKKAIDETKLLFGNLDNSSLN |
| | | GIYIQNDRSVINLSNSMFGSWSVIEDLWNKNYDSVNSNSRIKDIQKRED |
| | | KRKKAYKAEKKLSLSFLQVLISNSENDEIRKKSIVDYYKTSLMQLTDNL |
| | | SDKYNEAAPLLNENYSNEKGLKNDDKSISLIKNFLDAIKEIEKFIKPLS |
| | | ETNITGEKNDLFYSQFTPLLDNISRIDILYDKVRNYVTQKPESTDKIKL |
| | | NFGNYQLLNGWDKDKEREYGAVLLCKDEKYYLAIIDKSNNRILENIDFQ |
| | | DCDESDCYEKIIYKLLPTPNKMLPKVFFAKKHKKLLSPSDEILKIYKNG |
| | | TFKKGDKESLDDCHKLIDFYKESFKKYPKWLIYNFKFKKINGYNDIREF |
| | | YNDVALQGYNISKMKIPTSFIDKLVDEGKIYLFQLYNKDESPHSKGTPN |
| | | LHTLYFKMLEDERNLEDVVYRLNGEAEMFYRPASIKYDKPTHPKNTPIK |
| | | NKNTLNDKRASTFPYDLIKDKRYTKWQFSLHFPITMNFKDPDKAMINDD |
| | | VRNLLKSCNNNFIIGIDRGERNLLYVSVINSNGAIIYQHSLNIIGNKEK |
| | | GKTYETNYREKLATREKDRTEQRRNWKAIESIKELKEGYISQAVHVICQ |
| | | LVVKYDAIIVMEKLTDGFKRGRTKFEKQVYQKFEKMLIDKLNYYVDKKL |
| | | DPDEEGGLLHAYQLTNKLESFDKLGTQSGFIFYVRPDFTSKIDPVTGFV |
| | | NLLYPRYEKIDKAKDMISREDDIRYNAGEDFFEFDIDYDKFPKTASDYR |
| | | KKWTICINGERIEAFRNPANNNEWSYRTIILAEKFKELEDNNSINYRDS |
| | | DDLKAEILSQTKGKFFEDFFKLLRLTLQMRNSNPETGEDRILSPVKDKN |
| | | GNFYDSSKYDEKSKLPCDADANGAYNIARKGLWIVEQFKKADNVSTVEP |
| | | VIHNDKWLKFVQENDMANN |
| ART32 | 32 | KTGLDKLKDYAEIYYHCNTDADRKRLNKCASELRKEIVKNEKNRDEYNK |
| | | LFDKRMIEIVLPKHLKNEDEKEVVASFKNFTTYFTGFFTNRKNMYSDGE |
| | | ESTAIAYRCINENLPKHLDNVKAFEKAISKLSKNAIDDLDATYSGLCGT |
| | | NLYDVFTVDYENELLPQSGITEYNKIIGGYTTSDGTKVKGINEYINLYN |
| | | QQVSKRDKIPNLQILYKQILSESEKVSFIPPKFEDDNELLSAVSEFYAN |
| | | DETFDEMPLKKAIDETKLLFGNLDNSSLNGIYIQNDRSVTNLSNSMEGS |
| | | WSVIEDLWNKNYDSVNSNSRIKDIQKREDKRKKAYKAEKKLSLSFLQVL |
| | | ISNSENNEIREKSIVDYYKTSLMQLTDNLSDKYNEVAPLLNENYSNEKG |
| | | LKNDDKSISLIKNFLDAIKEIEKFIKPLSETNITGEKNDLFYSQFTPLL |
| | | DNISRIDILYDKVRNYVTQKPFSTDKIKLNFGNYQLLNGWDKDKEREYG |
| | | AVLLCRDEKYYLAIIDKSNNRILENIDFQDCDESDCYEKIIYKLLPTPN |
| | | KMLPKVFFAKKHKKLLSPSDEILKIRKNGTFKKGDKFSLDDCHKLIDFY |
| | | KESFKKYPNWLIYNFKFKKTNEYNDIREFYNDVALQGYNISKMKIPTSF |
| | | IDKLVDEGKIYLFQLYNKDESPHSKGTPNLHTLYFKMLEDERNLEDVVY |
| | | KLNGEAKMFYRPASIKYDKPTHPKNTPIKNKNTLNDKKASTFPYDLIKD |
| | | KRYTKWQFSLHESITMNFKAPDKAMINDDVRNLLKSCNNNFIIGIDRGE |
| | | RNLLYVSVIDSNGAIIYQHSLNIIGNKEKGKTYETNYREKLATREKERT |
| | | EQRRNWKAIESIKELKEGYISQAVHVICQLVVKYDAIIVMEKLTDGEKR |
| | | GRTKFEKQVYQKFEKMLIDKLNYYVDKKLDPDEEGGLLHAYQLTNKLES |
| | | FDKLGTQSGFIFYVRPDFTSKIDPVTGFVNLLYPRYENIDKAKDMISRF |
| | | DDIRYNAGEDFFEFDIDYDKFPKTASDYRKKWTICINGERIEAFRNPAN |
| | | NNEWSYRTIILAEKFKELFDNNSINYRDSDDLKAEILSQTKGKFFEDFF |
| | | KLLRLTLQMRNSNPETGEDRILSPVKDKNGNFYDSSKYDEKSKLPCDAD |
| | | ANGAYNIARKGLWIVEQFKKSDNVSTVEPVIHNDKWLKFVQENDMANN |
| ART33 | 33 | MSININKESDECRKIDFFTDLYNIQKTLRESLIPIGATADNFEFKGRLS |
| | | KEKDLLDSAKRIKEYISKYLADESDICLSQPVKLKHLDEYYELYITKDR |
| | | DEQKFKSVEEKLRKELADLLKEILKRLNKKILSDYLPEYLEDDEKALED |
| | | IANLSSFSTYFNSYYDNCKNMYTDKEQSTAIPYRCINDNLPKFIDNMKA |
| | | YEKALEELKPSDLEELRNNFKGVYDTTVDDMFTLDYFNCVLSQSGIDSY |
| | | NAIIGNDKVKGINEYINLHNQTAEQGHKVPNLKRLYKQIGSQKKTISFL |
| | | PSKFESDNELLKAVYDFYNTGDAEKNFTALKDTITEFEKIFDNLSEYNL |
| | | DGVFVRNDISLTNLSQSMENDWSVERNLWNDQYDKVNNPEKAKDIDKYN |
| | | DKRHKVYKKSESFSINQLQELIATTLEEDINSKKITDYFSCDEHRVTTE |
| | | VENKYQLVKDLLSSDYPKNKNLKTSEEDVALIKDELDSVKSLESFVKIL |
| | | TGTGKESGKDELFYGSFTKWFDQLRYIDKLYDKVRNYITEKPYSLDKIK |
| | | LSFDNPQFLGGWQHSKETDYSAQLFMKDGLYYLGVMDKETKREFKTQYN |
| | | TPENDSDTMVKIEYNQIPNPGRVIQNLMLVDGKIVKKNGRKNADGVNAV |
| | | LEELKNQYLPENINRIRKTESYKTTSNNENKDDLKAYLEYYIARTKEYY |
| | | CKYNFVFKSADEYGSFNEFVDDVNNQAYQITKVKVSEKQLLSLVEQGKL |
| | | YLFKIYNKDFSEYSKGKKNLHTMYFQMLFDDRNLENLVYKLQGGAEMFY |
| | | RPASIKKDSEFKHDANVEIIKRTCEDKVNDKDNPTDDEKAKYYSKEDYD |
| | | IVKNKRFTKDQFSLHLTLAMNCNQPDHYWLNNDVRELLKKSNKNHIIGI |
| | | DRGERNLIYVTIINSDGVIVDQINENIIENSYNGKKYKTDYQKKLNQRE |
| | | EDRQKARKTWKTIETIKELKDGYISQVVHQICKLIVQYDAIVVMENING |
| | | GFKRGRTKVEKQVYQKFETMLINKLNYYVDKGTDYKECGGLLKAYQLTN |
| | | KFETFERIGKQSGIIFYVDPYLTSKIDPVTGFANLLYPKYETIPKTHNF |
| | | ISNIDDIRYNQSEDYFEFDIDYDKFPQGSYNYRKKWTICSYGNRIKYYK |
| | | DSRNKTASVVVDITEKFKETFTNAGIDFVNDNIKEKLLLVNSKELLKSF |
| | | MDTLKLTVQLRNSEINSDVDYIISPIKDRNGNFYYSENYKKSNNEVPSQ |
| | | PQDGDANGAYNIARKGLMIINKLKKADDVTNNELLKISKKEWLEFAQKG |
| | | DLGE |
| ART34 | 34 | MKATSIWDNFTRKYSVSKTLRFELRPVGKTEENIVKKEIIDAEWISGKN |
| | | IPKGTDADRARDYKIVKKLLNQLHILFINQALSSENVKEFEKEDKKSKT |
| | | FVAWSDLLATHEDNWIQYTRDKSNSTVLKSLEKSKKDLYSKLGKLLNSK |
| | | ANAWKAEFISYHKIKSPDNIKIRLSASNVQILFGNTSDPIQLLKYQIEL |
| | | DNIKFLKDDGSEYTTKELADLLSTFEKFGTYFSGENQNRANVYDIDGEI |
| | | STSIAYRLENQNIEFFFQNIKRWEQFTSSIGHKEAKENLKLVQWDIQSK |
| | | LKELDMEIVQPRENLKFEKLLTPQSFIYLLNQEGIDAFNTVLGGIPAEV |
| | | KAEKKQGVNELINLTRQKLNEDKRKFPSLQIMYKQIMSERKINFIDQYE |
| | | DDVEMLKEIQEFSNDWNEKKKRHSASSKEIKESAIAYIQREFHETEDSL |
| | | EERATVKEDFYLSEKSIQNLSIDIFGGYNTIHNLWYTEVEGMLKSGERP |
| | | LTRVEKEKLKKQEYISFAQIERLISKHSQQYLDSTPKEANDRSLEKEKW |
| | | KKTFKNGFKVSEYTNLKLNELISEGETFQKIDQETGKETTIKIPGLFES |
| | | YENAILVESIKNQSLGTNKKESVPSIKEYLDSCLRLSKFIESFLVNSKD |
| | | LKEDQSLDGCSDFQNTLTQWLNEEFDVFILYNKVRNHVTKKPGNTDKIK |
| | | INFDNATLLDGWDVDKEAANFGFLLKKADNYYLGIADSSFNQDLKYENE |
| | | GERLDEIEKNRKNLEKEESKNISKIDQEKVKKYKEVIDDLKAISNLNKG |
| | | RYSKAFYKQSKFTTLIPKCTTQLNEVIEHFKKEDTDYRIENKKFAKPFI |
| | | ITKEVFLLNNTVYDTATKKFTLKIGEDEDTKGLKKFQIGYYRATDDKKG |
| | | YESALRNWITFCIEFTKSYKSCLNYNYSSLKSVSEYKSLDEFYKDLNGI |
| | | GYTIDFVDISEEYINKKINEGKLYLFQIYNKDESEKSKGKENLHTTYWK |
| | | LLFDSKNLEDVVIKLNGQAEVFFRPASIHEKEKITHEKNQEIQNKNPNA |
| | | VKKTSKFEYDIIKDNRFTKNKFLFHCPITLNFKADGNPYVNNEVQENIA |
| | | KNPNVNIIGIDRGEKHLLYFTVINQQGQILDAGSLNSIKSEYKDKNQQS |
| | | VSFETPYHKILDKKESERKEARESWQEIENIKELKAGYLSHVVHQLSNL |
| | | IVKYNAIVVLEDLNKGFKRGRFKVEKQVYQKFEKSLIEKLNYLVEKDRK |
| | | ESNEPGHHLNAYQLTNKELSFERLGKQSGVLFYATASYTSKVDPVTGEM |
| | | QNIYDPYHKEKTREFYKNFTKIVYNGNYFEFNYDLNSVKPDSEEKRYRT |
| | | NWTVCSCVIRSEYDSNSKTQKTYNVNDQLVKLFEDAKIKIENGNDLKST |
| | | ILEQDDKFIRDLHFYFIAIQKMRVVDSKIEKGEDSNDYIQSPVYPFYCS |
| | | KEIQPNKKGFYELPSNGDSNGAYNIARKGIVILDKIRLRVQIEKLFEDG |
| | | TKIDWQKLPNLISKVKDKKLLMTVFEEWAELTHQGEVQQGDLLGKKMSK |
| | | KGEQFAEFIKGLNVTKEDWEIYTQNEKVVQKQIKTWKLESNST |
| ART35 | 35 | MKAINEYYKQLGAYCREEGKEKDDFFKRIDGAYCAISHLFFGEHGEIAQ |
| | | SDSDVELIQKLLEAYKGLQRFIKPLLGHGDEADKDNEFDAKLRKVWDEL |
| | | DIITPLYDKVRNWLSRKIYNPEKIKLCFENNGKLLSGWVDSRTKSDNGT |
| | | QYGGYIFRKKNEIGEYDFYLGISADTKLFRRDAAISYDDGMYERLDYYQ |
| | | LKSKTLLGNSYVGDYGLDSMNLLSAFKNAAVKFQFEKEVVPKDKENVPK |
| | | YLKRLKLDYAGFYQILMNDDKVVDAYKIMKQHILATLTSSIRVPAAIEL |
| | | ATQKELGIDELIDEIMNLPSKSFGYFPIVTAAIEEANKRENKPLFLFKM |
| | | SNKDLSYAATASKGLRKGRGTENLHSMYLKALLGMTQSVEDIGSGMVFF |
| | | RHQTKGLAETTARHKANEFVANKNKLNDKKKSIFGYEIVKNKRFTVDKY |
| | | LFKLSMNLNYSQPNNNKIDVNSKVREIISNGGIKNIIGIDRGERNLLYL |
| | | SLIDLKGNIVMQKSLNILKDDHNAKETDYKGLLTEREGENKEARRNWKK |
| | | IANIKDLKRGYLSQVVHIISKMMVEYNAIVVLEDLNPGFIRGRQKIERN |
| | | VYEQFERMLIDKLNFYVDKHKGANETGGLLHALQLTSEFKNEKKSEHQN |
| | | GCLFYIPAWNTSKIDPATGFVNLENTKYTNAVEAQEFFSKEDEIRYNEE |
| | | KDWFEFEFDYDKFTQKAHGTRTKWTLCTYGMRLRSFKNSAKQYNWDSEV |
| | | VALTEEFKRILGEAGIDIHENLKDAICNLEGKSQKYLEPLMQFMKLLLQ |
| | | LRNSKAGTDEDYILSPVADENGIFYDSRSCGDQLPENADANGAYNIARK |
| | | GLMLIEQIKNAEDLNNVKEDISNKAWLNFAQQKPYKNGMKAINEYYKQL |
| | | GAYCREEGKEKDDFFKRIDGAYCAISHLFFGEHGEIAQSDSDVELIQKL |
| | | LEAYKGLQRFIKPLLGHGDEADKDNEFDAKLRKVWDELDIITPLYDKVR |
| | | NWLSRKIYNPEKIKLCFENNGKLLSGWVDSRTKSDNGTQYGGYIFRKKN |
| | | EIGEYDFYLGISADTKLERRDAAISYDDGMYERLDYYQLKSKTLLGNSY |
| | | VGDYGLDSMNLLSAFKNAAVKFQFEKEVVPKDKENVPKYLKRLKLDYAG |
| | | FYQILMNDDKVVDAYKIMKQHILATLTSSIRVPAAIELATQKELGIDEL |
| | | IDEIMNLPSKSFGYFPIVTAAIEEANKRENKPLFLFKMSNKDLSYAATA |
| | | SKGLRKGRGTENLHSMYLKALLGMTQSVEDIGSGMVFFRHQTKGLAETT |
| | | ARHKANEFVANKNKLNDKKKSIFGYEIVKNKRFTVDKYLFKLSMNLNYS |
| | | QPNNNKIDVNSKVREIISNGGIKNIIGIDRGERNLLYLSLIDLKGNIVM |
| | | QKSLNILKDDHNAKETDYKGLLTEREGENKEARRNWKKIANIKDLKRGY |
| | | LSQVVHIISKMMVEYNAIVVLEDLNPGFIRGRQKIERNVYEQFERMLID |
| | | KLNFYVDKHKGANETGGLLHALQLTSEFKNFKKSEHQNGCLFYIPAWNT |
| | | SKIDPATGFVNLENTKYTNAVEAQEFFSKEDEIRYNEEKDWFEFEFDYD |
| | | KFTQKAHGTRTKWTLCTYGMRLRSFKNSAKQYNWDSEVVALTEEFKRIL |
| | | GEAGIDIHENLKDAICNLEGKSQKYLEPLMQFMKLLLQLRNSKAGTDED |
| | | YILSPVADENGIFYDSRSCGDQLPENADANGAYNIARKGLMLIEQIKNA |
| | | EDLNNVKFDISNKAWLNFAQQKPYKNG |
| ART11 | 36 | MYYQGLTKLYPISKTIRNELIPVGKTLEHIRMNNILEADIQRKSDYERV |
| * | | KKLMDDYHKQLINESLQDVHLSYVEEAADLYLNASKDKDIVDKESKCQD |
| | | KLRKEIVNLLKSHENFPKIGNKEIIKLLQSLSDTEKDYNALDSFSKFYT |
| | | YFTSYNEVRKNLYSDEEKSSTAAYRLINENLPKELDNIKAYSIAKSAGV |
| | | RAKELTEEEQDCLEMTETFERTLTQDGIDNYNELIGKLNFAINLYNQQN |
| | | NKLKGFRKVPKMKELYKQILSEREASFVDEFVDDEALLINVESESAHIK |
| | | EFLESDSLSRFAEVLEESGGEMVYIKNDTSKTTFSNIVEGSWNVIDERL |
| | | AEEYDSANSKKKKDEKYYDKRHKELKKNKSYSVEKIVSLSTETEDVIGK |
| | | YIEKLQADIIAIKETREVFEKVVLKEHDKNKSLRKNTKAIEAIKSELDT |
| | | IKDFERDIKLISGSEHEMEKNLAVYAEQENILSSIRNVDSLYNMSRNYL |
| | | TQKPFSTEKFKLNFNRATLLNGWDKNKETDNLGILLVKEGKYYLGIMNT |
| | | KANKSFVNPPKPKTDNVYHKVNYKLLPGPNKMLPKVFFAKSNLEYYKPS |
| | | EDLLAKYQAGTHKKGENFSLEDCHSLISFFKDSLEKHPDWSEFGFKESD |
| | | TKKYDDLSGFYREVEKQGYKITYTDIDVEYIDSLVEKDELYFFQIYNKD |
| | | FSPYSKGNYNLHTLYLTMLEDERNLRNVVYKLNGEAEVFYRPASIGKDE |
| | | LIIHKSGEEIKNKNPKRAIDKPTSTFEYDIVKDRRYTKDKFMLHIPVTM |
| | | NFGVDETRRENEVVNDAIRGDDKVRVIGIDRGERNLLYVVVVDSDGTIL |
| | | EQISLNSIINNEYSIETDYHKLLDEKEGDRDRARKNWTTIENIKELKEG |
| | | YLSQVVNVIAKLVLKYDAIICLEDLNFGFKRGRQKVEKQVYQKFEKMLI |
| | | DKLNYLVIDKSRSQENPEEVGHVLNALQLTSKFTSFKELGKQTGIIYYV |
| | | PAYLTSKIDPTTGFANLFYVKYESVEKSKDFENREDSICENKVAGYFEF |
| | | SFDYKNFTDRACGMRSKWKVCTNGERIIKYRNEEKNSSEDDKVIVLTEE |
| | | FKKLFNEYGIAFNDCMDLTDAINAIDDASFFRKLTKLFQQTLQMRNSSA |
| | | DGSRDYIISPVENDNGEFENSEKCDKSKPKDADANGAFNIARKGLWVLE |
| | | QLYNSSSGEKLNLAMTNAEWLEYAQQHTI |

In certain embodiments, a Cas nuclease comprises ABW1 (SEQ ID NO: 3), ABW2 (SEQ ID NO: 16), ABW3 (SEQ ID NO: 29), ABW4 (SEQ ID NO: 42), ABW5 (SEQ ID NO: 55), ABW6 (SEQ ID NO: 68), ABW7 (SEQ ID NO: 81), ABW8 (SEQ ID NO: 94), or ABW9 (SEQ ID NO: 107) (all SEQ ID NOs for ABW 1-9 and variants thereof from International (PCT) Application Publication No. WO 2021/108324), or variants thereof, such as any one of variants 1-10 of ABW1 (SEQ ID NOs: 4-13, respectively), any one of variants 1-10 of ABW2 (SEQ ID NOs: 17-26, respectively), any one of variants 1-10 of ABW3 (SEQ ID NOs: 30-39, respectively), any one of variants 1-10 of ABW4 (SEQ ID NOs: 43-52, respectively), any one of variants 1-10 of ABW5 (SEQ ID NOs: 56-65, respectively), any one of variants 1-10 of ABW6 (SEQ ID NOs: 69-78, respectively), any one of variants 1-10 of ABW7 (SEQ ID NOs: 82-91, respectively), any one of variants 1-10 of ABW8 (SEQ ID NOs: 95-104, respectively), any one of variants 1-10 of ABW9 (SEQ ID NOs: 108-117, respectively). ABW1-ABW9, and variants thereof are known in the art and are described in International (PCT) Application Publication No. WO 2021/108324.

More type V-A Cas nucleases and their corresponding naturally occurring CRISPR-Cas systems can be identified by computational and experimental methods known in the art, e.g., as described in U.S. Pat. No. 9,790,490 and Shmakov et al. (2015) MOL. CELL, 60:385. Exemplary computational methods include analysis of putative Cas proteins by homology modeling, structural BLAST, PSI-BLAST, or HHPred, and analysis of putative CRISPR loci by identification of CRISPR arrays. Exemplary experimental methods include in vitro cleavage assays and in-cell nuclease assays (e.g., the Surveyor assay) as described in Zetsche et al. (2015) CELL, 163:759.

In certain embodiments, the Cas protein is a Cas nuclease that directs cleavage of one or both strands at the target locus, such as the target strand (i.e., the strand having the target nucleotide sequence that is at least partially complementary to and can hybridize with a single guide nucleic acid or dual guide nucleic acids) and/or the non-target strand. In certain embodiments, the Cas nuclease directs cleavage of one or both strands within about 1, 2, 3, 4, 5, 6, 7, 8, 9, 10, 15, 20, 25, 50, 100, 200, 500, or more nucleotides from the first or last nucleotide of the target nucleotide sequence or its complementary sequence. In certain embodiments, the cleavage is staggered, i.e. generating sticky ends. In certain embodiments, the cleavage generates a staggered cut with a 5′ overhang. In certain embodiments, the cleavage generates a staggered cut with a 5′ overhang of 1 to 5 nucleotides, e.g., of 4 or 5 nucleotides. In certain embodiments, the cleavage site is distant from the PAM, e.g., the cleavage occurs after the 18th nucleotide on the non-target strand and after the 23rd nucleotide on the target strand.

In certain embodiments, a composition provided herein comprises a Cas nuclease that a compatible guide nucleic acid (gNA), e.g., a gRNA, is capable of activating. In certain embodiments, a composition provided herein further comprises a Cas protein that is related to the Cas nuclease that a compatible guide nucleic acid (gNA), e.g., a gRNA, is capable of activating. For example, in certain embodiments, a Cas protein comprises an amino acid sequence at least 80% (e.g., at least 85%, at least 90%, at least 91%, at least 92%, at least 93%, at least 94%, at least 95%, at least 96%, at least 97%, at least 98%, or at least 99%) identical to the Cas nuclease amino acid sequence. In certain embodiments, a Cas protein comprises a nuclease-inactive mutant of the Cas nuclease. In certain embodiments, a Cas protein further comprises an effector domain.

In certain embodiments, a Cas protein lacks substantially all DNA cleavage activity. Such a Cas protein can be generated, e.g., by introducing one or more mutations to an active Cas nuclease (e.g., a naturally occurring Cas nuclease). A mutated Cas protein is considered to lack substantially all DNA cleavage activity when the DNA cleavage activity of the protein has no more than about 25%, 10%, 5%, 1%, 0.1%, 0.01%, or less of the DNA cleavage activity of the corresponding non-mutated form, for example, nil or negligible as compared with the non-mutated form. Thus, a Cas protein may comprise one or more mutations (e.g., a mutation in the RuvC domain of a type V-A Cas protein) and be used as a generic DNA binding protein with or without fusion to an effector domain. Exemplary mutations include D908A, E993A, and D1263A with reference to the amino acid positions in AsCpf1: D832A, E925A, and D1180A with reference to the amino acid positions in LbCpf1; and D917A, E1006A, and D1255A with reference to the amino acid position numbering of the FnCpf1. More mutations can be designed and generated according to the crystal structure described in Yamano et al. (2016) CELL, 165:949.

It is understood that a Cas protein, rather than losing nuclease activity to cleave all DNA, may lose the ability to cleave only the target strand or only the non-target strand of a double-stranded DNA, thereby being functional as a nickase (see, Gao et al. (2016) CELL RES., 26:901). Accordingly, in certain embodiments, a Cas nuclease is a Cas nickase. In certain embodiments, a Cas nuclease has the activity to cleave the non-target strand but lacks substantially the activity to cleave the target strand, e.g., by a mutation in the Nuc domain. In certain embodiments, a Cas nuclease has the cleavage activity to cleave the target strand but lacks substantially the activity to cleave the non-target strand.

In certain embodiments, a Cas nuclease has the activity to cleave a double-stranded DNA and result in a double-strand break.

Cas proteins that lack substantially all DNA cleavage activity or have the ability to cleave only one strand may also be identified from naturally occurring systems. For example, certain naturally occurring CRISPR-Cas systems may retain the ability to bind the target nucleotide sequence but lose entire or partial DNA cleavage activity in eukaryotic (e.g., mammalian or human) cells. Such type V-A proteins are disclosed, for example, in Kim et al. (2017) ACS SYNTH. BIOL. 6 (7): 1273-82 and Zhang et al. (2017) CELL DISCOV. 3:17018.

The activity of a Cas protein (e.g., Cas nuclease) can be altered, e.g., by creating an engineered Cas protein. In certain embodiments, altered activity of an engineered Cas protein comprises increased targeting efficiency and/or decreased off-target binding. While not wishing to be bound by theory, it is hypothesized that off-target binding can be recognized by the Cas protein, for example, by the presence of one or more mismatches between the spacer sequence and the target nucleotide sequence, which may affect the stability and/or conformation of the CRISPR-Cas complex. In certain embodiments, altered activity comprises modified binding, e.g., increased binding to the target locus (e.g., the target strand or the non-target strand) and/or decreased binding to off-target loci. In certain embodiments, altered activity comprises altered charge in a region of the protein that associates with a single guide nucleic acid or dual guide nucleic acids. In certain embodiments, altered activity of an engineered Cas protein comprises altered charge in a region of the protein that associates with the target strand and/or the non-target strand. In certain embodiments, altered activity of an engineered Cas protein comprises altered charge in a region of the protein that associates with an off-target locus. The altered charge can include decreased positive charge, decreased negative charge, increased positive charge, or increased negative charge. For example, decreased negative charge and increased positive charge may generally strengthen binding to the nucleic acid(s) whereas decreased positive charge and increased negative charge may weaken binding to the nucleic acid(s). In certain embodiments, altered activity comprises increased or decreased steric hindrance between the protein and a single guide nucleic acid or dual guide nucleic acids. In certain embodiments, altered activity comprises increased or decreased steric hindrance between the protein and the target strand and/or the non-target strand. In certain embodiments, altered activity comprises increased or decreased steric hindrance between the protein and an off-target locus. In certain embodiments, a modification or mutation comprises one or more substitutions of Lys, His, Arg, Glu, Asp, Ser, Gly, and/or Thr. In certain embodiments, a modification or mutation comprises one or more substitutions with Gly, Ala, Ile, Glu, and/or Asp. In certain embodiments, modification or mutation comprises one or more amino acid substitutions in the groove between the WED and RuvC domain of the Cas protein (e.g., a type V-A Cas protein).

In certain embodiments, altered activity of an engineered Cas protein comprises increased nuclease activity to cleave the target locus. In certain embodiments, altered activity of an engineered Cas protein comprises decreased nuclease activity to cleave an off-target locus. In certain embodiments, altered activity of an engineered Cas protein comprises altered helicase kinetics. In certain embodiments, an engineered Cas protein comprises a modification that alters formation of the CRISPR complex.

In certain embodiments, a protospacer adjacent motif (PAM) or PAM-like motif directs binding of a Cas protein complex to a target locus. Many Cas proteins have PAM specificity. The precise sequence and length requirements for the PAM differ depending on the Cas protein used. PAM sequences are typically 2-5 base pairs in length and are adjacent to (but located on a different strand of target DNA from) the target nucleotide sequence. PAM sequences can be identified using any suitable method, such as testing cleavage, targeting, or modification of oligonucleotides having the target nucleotide sequence and different PAM sequences.

Exemplary PAM sequences are provided in Tables 2 and 3. In certain embodiments, a Cas protein comprises MAD7 and the PAM is TTTN, wherein N is A, C, G, or T. In certain embodiments, a Cas protein comprises MAD7 and the PAM is CTTN, wherein N is A, C, G, or T. In certain embodiments, a Cas protein comprises AsCpf1 and the PAM is TTTN, wherein N is A, C, G, or T. In certain embodiments, a Cas protein comprises FnCpf1 and the PAM is 5′ TTN, wherein N is A, C, G, or T. PAM sequences for certain other type V-A Cas proteins are disclosed in Zetsche et al. (2015) CELL, 163:759 and U.S. Pat. No. 9,982,279. Further, engineering of the PAM Interacting (PI) domain of a Cas protein may allow programing of PAM specificity, improve target site recognition fidelity, and/or increase the versatility of an engineered, non-naturally occurring system. Exemplary approaches to alter the PAM specificity of Cpf1 are described in Gao et al. (2017) NAT. BIOTECHNOL., 35:789.

In certain embodiments, an engineered Cas protein comprises a modification that alters the Cas protein specificity in concert with modification to targeting range. Cas mutants can be designed to have increased target specificity as well as accommodating modifications in PAM recognition, for example by choosing mutations that alter PAM specificity (e.g., in the PI domain) and combining those mutations with groove mutations that increase (or if desired, decrease) specificity for the on-target locus versus off-target loci. The Cas modifications described herein can be used to counter loss of specificity resulting from alteration of PAM recognition, enhance gain of specificity resulting from alteration of PAM recognition, counter gain of specificity resulting from alteration of PAM recognition, or enhance loss of specificity resulting from alteration of PAM recognition.

In certain embodiments, an engineered Cas protein comprises one or more nuclear localization signal (NLS) motifs. In certain embodiments, an engineered Cas protein comprises at least 2 (e.g., at least 3, at least 4, at least 5, at least 6, at least 7, at least 8, at least 9, or at least 10) NLS motifs. Non-limiting examples of NLS motifs include: the NLS of SV40 large T-antigen, having the amino acid sequence of PKKKRKV (SEQ ID NO: 40): the NLS from nucleoplasmin, e.g., the nucleoplasmin bipartite NLS having the amino acid sequence of KRPAATKKAGQAKKKK (SEQ ID NO: 41); the c-myc NLS, having the amino acid sequence of PAAKRVKLD (SEQ ID NO: 42) or RQRRNELKRSP (SEQ ID NO: 43); the hRNPA1 M9 NLS, having the amino acid sequence of NQSSNFGPMKGGNFGGRSSGPYGGGGQYFAKPRNQGGY (SEQ ID NO: 44); the importin-α IBB domain NLS, having the amino acid sequence of RMRIZFKNKGKDTAELRRRRVEVSVELRKAKKDEQILKRRNV (SEQ ID NO: 45); the myoma T protein NLS, having the amino acid sequence of VSRKRPRP (SEQ ID NO: 46) or PPKKARED (SEQ ID NO: 47); the human p53 NLS, having the amino acid sequence of PQPKKKPL (SEQ ID NO: 48); the mouse c-abl IV NLS, having the amino acid sequence of SALIKKKKKMAP (SEQ ID NO: 49); the influenza virus NS1 NLS, having the amino acid sequence of DRLRR (SEQ ID NO: 50) or PKQKKRK (SEQ ID NO: 51); the hepatitis virus 8 antigen NLS, having the amino acid sequence of RKLKKKIKKL (SEQ ID NO: 52); the mouse Mxl protein NLS, having the amino acid sequence of REKKKFLKRR (SEQ ID NO: 53); the human poly (ADP-ribose) polymerase NLS, having the amino acid sequence of KRKGDEVDGVDEVAKKKSKK (SEQ ID NO: 54); the human glucocorticoid receptor NLS, having the amino acid sequence of RKCLQAGMNLEARKTKK (SEQ ID NO: 55), and synthetic NLS motifs such as PAAKKKKLD (SEQ ID NO: 56).

In general, the one or more NLS motifs are of sufficient strength to drive accumulation of the Cas protein in a detectable amount in the nucleus of a eukaryotic cell. The strength of nuclear localization activity may derive from the number of NLS motif(s) in the Cas protein, the particular NLS motif(s) used, the position(s) of the NLS motif(s), or a combination of these and/or other factors. In certain embodiments, an engineered Cas protein comprises at least 1 (e.g., at least 2, at least 3, at least 4, at least 5, at least 6, at least 7, at least 8, at least 9, or at least 10) NLS motif(s) at or near the N-terminus (e.g., within about 1, 2, 3, 4, 5, 10, 15, 20, 25, 30, 40, 50, or more amino acids along the polypeptide chain from the N-terminus). In certain embodiments, an engineered Cas protein comprises at least 1 (e.g., at least 2, at least 3, at least 4, at least 5, at least 6, at least 7, at least 8, at least 9, or at least 10) NLS motif(s) at or near the C-terminus (e.g., within about 1, 2, 3, 4, 5, 10, 15, 20, 25, 30, 40, 50, or more amino acids along the polypeptide chain from the C-terminus). In certain embodiments, an engineered Cas protein comprises at least 1 (e.g., at least 2, at least 3, at least 4, at least 5, at least 6, at least 7, at least 8, at least 9, or at least 10) NLS motif(s) at or near the C-terminus and at least 1 (e.g., at least 2, at least 3, at least 4, at least 5, at least 6, at least 7, at least 8, at least 9, or at least 10) NLS motif(s) at or near the N-terminus. In certain embodiments, the engineered Cas protein comprises one, two, or three NLS motifs at or near the C-terminus. In certain embodiments, the engineered Cas protein comprises one NLS motif at or near the N-terminus and one, two, or three NLS motifs at or near the C-terminus. In certain embodiments, the engineered Cas protein comprises a nucleoplasmin NLS at or near the C-terminus.

Detection of accumulation in the nucleus may be performed by any suitable technique. For example, a detectable marker may be fused to a nucleic acid-targeting protein, such that location within a cell may be visualized. Cell nuclei may also be isolated from cells, the contents of which may then be analyzed by any suitable process for detecting the protein, such as immunohistochemistry, Western blot, or enzyme activity assay. Accumulation in the nucleus may also be determined indirectly, such as by an assay that detects the effect of the nuclear import of a Cas protein complex (e.g., assay for DNA cleavage or mutation at the target locus, or assay for altered gene expression activity) as compared to a control not exposed to the Cas protein or exposed to a Cas protein lacking one or more of the NLS motifs.

A Cas protein may comprise a chimeric Cas protein, e.g., a Cas protein having enhanced function by being a chimera. Chimeric Cas proteins may be new Cas proteins containing fragments from more than one naturally occurring Cas protein or variants thereof. For example, fragments of multiple type V-A Cas homologs (e.g., orthologs) may be fused to form a chimeric Cas protein. In certain embodiments, a chimeric Cas protein comprises fragments of Cpf1 orthologs from multiple species and/or strains.

In certain embodiments, a Cas protein comprises one or more effector domains. The one or more effector domains may be located at or near the N-terminus of the Cas protein and/or at or near the C-terminus of the Cas protein. In certain embodiments, an effector domain comprised in the Cas protein is a transcriptional activation domain (e.g., VP64), a transcriptional repression domain (e.g., a KRAB domain or an SID domain), an exogenous nuclease domain (e.g., FokI), a deaminase domain (e.g., cytidine deaminase or adenine deaminase), or a reverse transcriptase domain (e.g., a high fidelity reverse transcriptase domain). Other activities of effector domains include but are not limited to methylase activity, demethylase activity, transcription release factor activity, translational initiation activity, translational activation activity, translational repression activity, histone modification (e.g., acetylation or demethylation) activity, single-stranded RNA cleavage activity, double-strand RNA cleavage activity, single-strand DNA cleavage activity, double-strand DNA cleavage activity, and nucleic acid binding activity.

In certain embodiments, a Cas protein comprises one or more protein domains that enhance homology-directed repair (HDR) and/or inhibit non-homologous end joining (NHEJ). Exemplary protein domains having such functions are described in Jayavaradhan et al. (2019) NAT. COMMUN. 10 (1): 2866 and Janssen et al. (2019) MOL. THER. NUCLEIC ACIDS 16:141-54. In certain embodiments, a Cas protein comprises a dominant negative version of p53-binding protein 1 (53BP1), for example, a fragment of 53BP1 comprising a minimum focus forming region (e.g., amino acids 1231-1644 of human 53BP1). In certain embodiments, a Cas protein comprises a motif that is targeted by APC-Cdh1, such as amino acids 1-110 of human Geminin, thereby resulting in degradation of the fusion protein during the HDR non-permissive G1 phase of the cell cycle.

In certain embodiments, a Cas protein comprises an inducible or controllable domain. Non-limiting examples of inducers or controllers include light, hormones, and small molecule drugs. In certain embodiments, a Cas protein comprises a light inducible or controllable domain. In certain embodiments, a Cas protein comprises a chemically inducible or controllable domain.

In certain embodiments, a Cas protein comprises a tag protein or peptide for ease of tracking and/or purification. Non-limiting examples of tag proteins and peptides include fluorescent proteins (e.g., green fluorescent protein (GFP), YFP, RFP, CFP, mCherry, tdTomato), HIS tags (e.g., 6×His tag, or gly-6×His: 8×His, or gly-8×His), hemagglutinin (HA) tag, FLAG tag, 3×FLAG tag, and Myc tag.

In certain embodiments, a Cas protein is conjugated to a non-protein moiety, such as a fluorophore useful for genomic imaging. In certain embodiments, a Cas protein is covalently conjugated to the non-protein moiety. The terms “CRISPR-Associated protein,” “Cas protein,” “Cas,” “CRISPR-Associated nuclease,” and “Cas nuclease” are used herein to include such conjugates despite the presence of one or more non-protein moieties.

### B. Guide Nucleic Acids

A guide nucleic acid can be a single gNA (sgNA, e.g., sgRNA), in which the gNA is a single polynucleotide, or a dual gNA (e.g., dual gRNA), in which the gNA comprises two separate polynucleotides (these can in some cases be covalently linked, but not via a conventional internucleotide linkage). In certain embodiments, a single guide nucleic acid is capable of activating a Cas nuclease alone (e.g., in the absence of a tracrRNA).

In general, a gNA comprises a modulator nucleic acid and a targeter nucleic acid. In a sgNA the modulator and targeter nucleic acids are part of a single polynucleotide. In a dual gNA the modulator and targeter nucleic acids are separate, e.g., not joined by a conventional nucleotide linkage, such as not joined at all. The targeter nucleic acid comprises a spacer sequence and a targeter stem sequence. The modulator nucleic acid comprises a modulator stem sequence and, generally, further nucleotides, such as nucleotides comprising a 5′ tail. The modulator stem sequence and targeter stem sequence can each comprise any suitable number of nucleotides and are of sufficient complementarity that they can hybridize. In a single gNA there may be additional NTs between the targeter stem sequence and the modulator stem sequence: these can, in certain cases, form secondary structure, such as a loop.

In certain embodiments, the guide nucleic acid comprises a targeter nucleic acid that, in combination with a modulator nucleic acid, is capable of binding a Cas protein. In certain embodiments, the guide nucleic acid comprises a targeter nucleic acid that, in combination with a modulator nucleic acid, is capable of activating a Cas nuclease. In certain embodiments, the system further comprises the Cas protein that the targeter nucleic acid and the modulator nucleic acid are capable of binding or the Cas nuclease that the targeter nucleic acid and the modulator nucleic acid are capable of activating.

It is contemplated that the single or dual guide nucleic acids need to be the compatible with a Cas protein (e.g., Cas nuclease) to provide an operative CRISPR system. For example, the targeter stem sequence and the modulator stem sequence can be derived from a naturally occurring crRNA capable of activating a Cas nuclease in the absence of a tracrRNA. Alternatively, the targeter stem sequence and the modulator stem sequence can be derived from a naturally occurring set of crRNA and tracrRNA, respectively, that are capable of activating a Cas nuclease. In certain embodiments, the nucleotide sequences of the targeter stem sequence and the modulator stem sequence are identical to the corresponding stem sequences of a stem-loop structure in such naturally occurring crRNA.

Guide nucleic acid sequences that are operative with a type II or type V Cas protein are known in the art and are disclosed, for example, in U.S. Pat. Nos. 9,790,490, 9,896,696, 10,113,179, and 10,266,850, and U.S. Patent Application Publication No. 2014/0242664. It is understood that these sequences are merely illustrative, and other guide nucleic acid sequences may also be used with these Cas proteins.

TABLE 2
Type V-A Cas Protein and Corresponding Single Guide Nucleic Acid Sequences

| Cas Protein | Scaffold Sequence¹ | PAM² |
|---|---|---|
| MAD7 (SEQ ID | UAAUUUCUACUCUUGUAGA (SEQ ID NO: 57), | 5′ TTTN |
| NO: 37) | AUCUACAACAGUAGA (SEQ ID NO: 58), | or 5′ |
| | AUCUACAAAAGUAGA (SEQ ID NO: 59), | CTTN |
| | GGAAUUUCUACUCUUGUAGA (SEQ ID NO: 60), | |
| | UAAUUCCCACUCUUGUGGG (SEQ ID NO: 61) | |
| MAD2 (SEQ ID | AUCUACAAGAGUAGA (SEQ ID NO: 62), | 5′ TTTN |
| NO: 38) | AUCUACAACAGUAGA (SEQ ID NO: 58), | |
| | AUCUACAAAAGUAGA (SEQ ID NO: 59), | |
| | AUCUACACUAGUAGA (SEQ ID NO: 63) | |
| AsCpf1 (SEQ | UAAUUUCUACUCUUGUAGA (SEQ ID NO: 57) | 5′ TTTN |
| ID NO: 3 of | | |
| WO | | |
| 2021/158918) | | |
| LbCpf1 (SEQ | UAAUUUCUACUAAGUGUAGA (SEQ ID NO: 64) | 5′ TTTN |
| ID NO: 4 of | | |
| WO | | |
| 2021/158918) | | |
| FnCpf1 (SEQ | UAAUUUUCUACUUGUUGUAGA (SEQ ID NO: 65) | 5′ TTN |
| ID NO: 5 of | | |
| WO | | |
| 2021/158918) | | |
| PbCpf1 (SEQ | AAUUUCUACUGUUGUAGA (SEQ ID NO: 66) | 5′ TTTC |
| ID NO: 6 of | | |
| WO | | |
| 2021/158918) | | |
| PsCpf1 (SEQ | AAUUUCUACUGUUGUAGA (SEQ ID NO: 66) | 5′ TTTC |
| ID NO: 7 of | | |
| WO | | |
| 2021/158918) | | |
| As2Cpf1 (SEQ | AAUUUCUACUGUUGUAGA (SEQ ID NO: 66) | 5′ TTTC |
| ID NO: 8 of | | |
| WO | | |
| 2021/158918) | | |
| McCpf1 (SEQ | GAAUUUCUACUGUUGUAGA (SEQ ID NO: 67) | 5′ TTTC |
| ID NO: 9 of | | |
| WO | | |
| 2021/158918) | | |
| Lb3Cpf1 (SEQ | GAAUUUCUACUGUUGUAGA (SEQ ID NO: 67) | 5′ TTTC |
| ID NO: 10 of | | |
| WO | | |
| 2021/158918) | | |
| EcCpf1 (SEQ | GAAUUUCUACUGUUGUAGA (SEQ ID NO: 67) | 5′ TTTC |
| ID NO: 11 of | | |
| WO | | |
| 2021/158918) | | |
| SmCsm1 (SEQ | GAAUUUCUACUGUUGUAGA (SEQ ID NO: 67) | 5′ TTTC |
| ID NO: 12 of | | |
| WO | | |
| 2021/158918) | | |
| SsCsm1 (SEQ | GAAUUUCUACUGUUGUAGA (SEQ ID NO: 67) | 5′ TTTC |
| ID NO: 13 of | | |
| WO | | |
| 2021/158918) | | |
| MbCsm1 (SEQ | GAAUUUCUACUGUUGUAGA (SEQ ID NO: 67) | 5′ TTTC |
| ID NO: 14 of | | |
| WO | | |
| 2021/158918) | | |
| ART2 (SEQ ID | GUCUAAAGGUACCACCAAAUUUCUACUGUUGUAGAU | 5′ TTTN |
| NO: 2 | (SEQ ID NO: 68) | or 5′ |
| | | NTTN |
| ART11 (SEQ ID | GCUUAGAACCUUUAAAUAAUUUCUACUAUUGUAGAU | 5′ TTTN |
| NO: 11 | (SEQ ID NO: 69) | or 5′ |
| | | NTTN |
| ART11* (SEQ | GCUUAGAACCUUUAAAUAAUUUCUACUAUUGUAGAU | 5′ TTTN |
| ID NO: 36 | (SEQ ID NO: 69) | or 5′ |
| | | NTTN |

¹The modulator sequence in the scaffold sequence is underlined; the targeter stem sequence in the scaffold sequence is bold-underlined. It is understood that a “scaffold sequence” listed herein constitutes a portion of a single guide nucleic acid. Additional nucleotide sequences, other than the spacer sequence, can be comprised in the single guide nucleic acid.
²In the consensus PAM sequences, N represents A, C, G, or T. Where the PAM sequence is preceded by “5′,” it means that the PAM is located immediately upstream of the target nucleotide sequence when using the non-target strand (i.e., the strand not hybridized with the spacer sequence) as the coordinate.

TABLE 3
Type V-A Cas Protein and Corresponding Dual Guide Nucleic Acid Sequences

| | | Targeter | |
|---|---|---|---|
| | | Stem | |
| Cas Protein | Modulator Sequence¹ | Sequence | PAM² |
| MAD7 (SEQ ID NO: | UAAUUUCUAC (SEQ ID NO: | GUAGA | 5′ TTTN |
| 37) | 70) | | or 5′ |
| | AUCUAC (SEQ ID NO: 71) | GUAGA | CTTN |
| | GGAAUUUCUAC (SEQ ID NO: | GUAGA | |
| | 72) | | |
| | UAAUUCCCAC (SEQ ID NO: | GUGGG | |
| | 73) | | |
| MAD2 (SEQ ID NO: | AUCUAC (SEQ ID NO: 71) | GUAGA | 5′ TTTN |
| 38) | | | |
| AsCpf1 (SEQ ID NO: | UAAUUUCUAC (SEQ ID NO: | GUAGA | 5′ TTTN |
| 3 of WO | 70) | | |
| 2021/158918) | | | |
| LbCpf1 (SEQ ID NO: | UAAUUUCUAC (SEQ ID NO: | GUAGA | 5′ TTTN |
| 4 of WO | 70) | | |
| 2021/158918) | | | |
| FnCpf1 (SEQ ID NO: | UAAUUUUCUACU (SEQ ID NO: | GUAGA | 5′ TTN |
| 5 of WO | 74) | | |
| 2021/158918) | | | |
| PbCpf1 (SEQ ID NO: | AAUUUCUAC (SEQ ID NO: 75) | GUAGA | 5′ TTTC |
| 6 of WO | | | |
| 2021/158918) | | | |
| PsCpf1 (SEQ ID NO: | AAUUUCUAC (SEQ ID NO: 75) | GUAGA | 5′ TTTC |
| 7 of WO | | | |
| 2021/158918) | | | |
| As2Cpf1 (SEQ ID | AAUUUCUAC (SEQ ID NO: 75) | GUAGA | 5′ TTTC |
| NO: 8 of WO | | | |
| 2021/158918) | | | |
| McCpf1 (SEQ ID NO: | GAAUUUCUAC (SEQ ID NO: | GUAGA | 5′ TTTC |
| 9 of WO | 76) | | |
| 2021/158918) | | | |
| Lb3Cpf1 (SEQ ID | GAAUUUCUAC (SEQ ID NO: | GUAGA | 5′ TTTC |
| NO: 10 of WO | 76) | | |
| 2021/158918) | | | |
| EcCpf1 (SEQ ID NO: | GAAUUUCUAC (SEQ ID NO: | GUAGA | 5′ TTTC |
| 11 of WO | 76) | | |
| 2021/158918) | | | |
| SmCsm1 (SEQ ID NO: | GAAUUUCUAC (SEQ ID NO: | GUAGA | 5′ TTTC |
| 12 of WO | 76) | | |
| 2021/158918) | | | |
| SsCsm1 (SEQ ID NO: | GAAUUUCUAC (SEQ ID NO: | GUAGA | 5′ TTTC |
| 13 of WO | 76) | | |
| 2021/158918) | | | |
| MbCsm1 (SEQ ID NO: | GAAUUUCUAC (SEQ ID NO: | GUAGA | 5′ TTTC |
| 14 of WO | 76) | | |
| 2021/158918) | | | |
| ART2 (SEQ ID NO: 2) | AAAUUUCUAC (SEQ ID NO: | GUAGA | 5′ TTTN |
| | 77) | | or 5′ |
| | | | NTTN |
| ART11 (SEQ ID NO: | UAAUUUCUAC (SEQ ID NO: | GUAGA | 5′ TTTN |
| 11) | 70) | | or 5′ |
| | | | NTTN |
| ART11* (SEQ ID NO: | UAAUUUCUAC (SEQ ID NO: | GUAGA | 5′ TTTN |
| 36) | 70) | | or 5′ |
| | | | NTTN |

¹It is understood that a “modulator sequence” listed herein may constitute the nucleotide sequence of a modulator nucleic acid. Alternatively, additional nucleotide sequences can be comprised in the modulator nucleic acid 5′ and/or 3′ to a ″modulator sequence″ listed herein.
²In the consensus PAM sequences, N represents A, C, G, or T. Where the PAM sequence is preceded by “5′,” it means that the PAM is located immediately upstream of the target nucleotide sequence when using the non-target strand (i.e., the strand not hybridized with the spacer sequence) as the coordinate.

In certain embodiments, a guide nucleic acid, in the context of a type V-A CRISPR-Cas system, comprises a targeter stem sequence listed in Table 3. The same targeter stem sequences, as a portion of scaffold sequences, are bold-underlined in Table 2.

In certain embodiments, a guide nucleic acid is a single guide nucleic acid that comprises, from 5′ to 3′, a modulator stem sequence, a loop sequence, a targeter stem sequence, and a spacer sequence. In certain embodiments, the targeter stem sequence in the single guide nucleic acid is listed in Table 2 as a bold-underlined portion of scaffold sequence, and the modulator stem sequence is complementary (e.g., 100% complementary) to the targeter stem sequence. In certain embodiments, the single guide nucleic acid comprises, from 5′ to 3′, a modulator sequence listed in Table 2 as an underlined portion of a scaffold sequence, a loop sequence, a targeter stem sequence a bold-underlined portion of the same scaffold sequence, and a spacer sequence. In certain embodiments, an engineered, non-naturally occurring system comprises a single guide nucleic acid comprising a scaffold sequence listed in Table 2. In certain embodiments, the system further comprises a Cas protein (e.g., Cas nuclease) comprising an amino acid sequence at least 30%, 40%, 50%, 60%, 70%, 80%, 85%, 90%, 91%, 92%, 93%, 94%, 95%, 96%, 97%, 98%, 99%, or 100% identical to the amino acid sequence set forth in the SEQ ID NO listed in the same line of Table 2. In certain embodiments, the system further comprises a Cas protein (e.g., Cas nuclease) comprising the amino acid sequence set forth in the SEQ ID NO listed in the same line of Table 2. In certain embodiments, the system is useful for targeting, editing, or modifying a nucleic acid comprising a target nucleotide sequence close or adjacent to (e.g., immediately downstream of) a PAM listed in the same line of Table 2 when using the non-target strand (i.e., the strand not hybridized with the spacer sequence) as the coordinate.

In certain embodiments, a guide nucleic acid, e.g., dual gNA, comprises a targeter guide nucleic acid that comprises, from 5′ to 3′, a targeter stem sequence and a spacer sequence. In certain embodiments, the targeter stem sequence in the targeter nucleic acid is listed in Table 3. In certain embodiments, an engineered, non-naturally occurring system comprises the targeter nucleic acid and a modulator stem sequence complementary (e.g., 100% complementary) to the targeter stem sequence. In certain embodiments, the modulator nucleic acid comprises a modulator sequence listed in the same line of Table 3. In certain embodiments, the system further comprises a Cas protein (e.g., Cas nuclease) comprising an amino acid sequence at least 30%, 40%, 50%, 60%, 70%, 80%, 85%, 90%, 91%, 92%, 93%, 94%, 95%, 96%, 97%, 98%, 99%, or 100% identical to the amino acid sequence set forth in the SEQ ID NO listed in the same line of Table 3. In certain embodiments, the system further comprises a Cas protein (e.g., Cas nuclease) comprising the amino acid sequence set forth in the SEQ ID NO listed in the same line of Table 3. In certain embodiments, the system is useful for targeting, editing, or modifying a nucleic acid comprising a target nucleotide sequence close or adjacent to (e.g., immediately downstream of) a PAM listed in the same line of Table 3 when using the non-target strand (i.e., the strand not hybridized with the spacer sequence) as the coordinate.

A single guide nucleic acid, the targeter nucleic acid, and/or the modulator nucleic acid can be synthesized chemically or produced in a biological process (e.g., catalyzed by an RNA polymerase in an in vitro reaction). Such reaction or process may limit the lengths of the single guide nucleic acid, targeter nucleic acid, and/or modulator nucleic acid. In certain embodiments, a single guide nucleic acid is no more than 100, 90, 80, 70, 60, 50, 40, 30, or 25 nucleotides in length. In certain embodiments, a single guide nucleic acid is at least 20, 25, 30, 40, 50, 60, 70, 80, or 90 nucleotides in length. In certain embodiments, the single guide nucleic acid is 20-100, 20-90, 20-80, 20-70, 20-60, 20-50, 20-40, 20-30, 20-25, 25-100, 25-90, 25-80, 25-70, 25-60, 25-50, 25-40, 25-30, 30-100, 30-90, 30-80, 30-70, 30-60, 30-50, 30-40, 40-100, 40-90, 40-80, 40-70, 40-60, 40-50, 50-100, 50-90, 50-80, 50-70, 50-60, 60-100, 60-90, 60-80, 60-70, 70-100, 70-90, 70-80, 80-100, 80-90, or 90-100 nucleotides in length. In certain embodiments, a targeter nucleic acid is no more than 100, 90, 80, 70, 60, 50, 40, 30, or 25 nucleotides in length. In certain embodiments, a targeter nucleic acid is at least 20, 25, 30, 40, 50, 60, 70, 80, or 90 nucleotides in length. In certain embodiments, the targeter nucleic acid is 20-100, 20-90, 20-80, 20-70, 20-60, 20-50, 20-40, 20-30, 20-25, 25-100, 25-90, 25-80, 25-70, 25-60, 25-50, 25-40, 25-30, 30-100, 30-90, 30-80, 30-70, 30-60, 30-50, 30-40, 40-100, 40-90, 40-80, 40-70, 40-60, 40-50, 50-100, 50-90, 50-80, 50-70, 50-60, 60-100, 60-90, 60-80, 60-70, 70-100, 70-90, 70-80, 80-100, 80-90, or 90-100 nucleotides in length. In certain embodiments, a modulator nucleic acid is no more than 100, 90, 80, 70, 60, 50, 40, 30, or 20 nucleotides in length. In certain embodiments, a modulator nucleic acid is at least 10, 15, 20, 25, 30, 40, 50, 60, 70, 80, or 90 nucleotides in length. In certain embodiments, the modulator nucleic acid is 10-100, 10-90, 10-80, 10-70, 10-60, 10-50, 10-40, 10-30, 10-20, 15-100, 15-90, 15-80, 15-70, 15-60, 15-50, 15-40, 15-30, 15-20, 20-100, 20-90, 20-80, 20-70, 20-60, 20-50, 20-40, 20-30, 25-100, 25-90, 25-80, 25-70, 25-60, 25-50, 25-40, 25-30, 30-100, 30-90, 30-80, 30-70, 30-60, 30-50, 30-40, 40-100, 40-90, 40-80, 40-70, 40-60, 40-50, 50-100, 50-90, 50-80, 50-70, 50-60, 60-100, 60-90, 60-80, 60-70, 70-100, 70-90, 70-80, 80-100, 80-90, or 90-100 nucleotides in length.

It is contemplated that the length of the duplex formed within the single guide nuclei acid or formed between the targeter nucleic acid and the modulator nucleic acid, e.g. in a dual gNA, may be a factor in providing an operative CRISPR system. In certain embodiments, the targeter stem sequence and the modulator stem sequence each consist of 4-10 nucleotides that base pair with each other. In certain embodiments, the targeter stem sequence and the modulator stem sequence each consist of 4-9, 4-8, 4-7, 4-6, 4-5, 5-10, 5-9, 5-8, 5-7, or 5-6 nucleotides that base pair with each other. In certain embodiments, the targeter stem sequence and the modulator stem sequence each consist of 4, 5, 6, 7, 8, 9, or 10 nucleotides. It is understood that the composition of the nucleotides in each sequence affects the stability of the duplex, and a C-G base pair confers greater stability than an A-U base pair. In certain embodiments, 20%-80%, 20%-70%, 20%-60%, 20%-50%, 20%-40%, 20%-30%, 30%-80%, 30%-70%, 30%-60%, 30%-50%, 30%-40%, 40%-80%, 40%-70%, 40%-60%, 40%-50%, 50%-80%, 50%-70%, 50%-60%, 60%-80%, 60%-70%, or 70%-80% of the base pairs are C-G base pairs.

In certain embodiments, the targeter stem sequence and the modulator stem sequence each consist of 5 nucleotides. As such, the targeter stem sequence and the modulator stem sequence form a duplex of 5 base pairs. In certain embodiments, 0-4, 0-3, 0-2, 0-1, 1-5, 1-4, 1-3, 1-2, 2-5, 2-4, 2-3, 3-5, 3-4, or 4-5 out of the 5 base pairs are C-G base pairs. In certain embodiments, 0, 1, 2, 3, 4, or 5 out of the 5 base pairs are C-G base pairs. In certain embodiments, the targeter stem sequence consists of 5′-GUAGA-3′ and the modulator stem sequence consists of 5′-UCUAC-3′. In certain embodiments, the targeter stem sequence consists of 5′-GUGGG-3′ and the modulator stem sequence consists of 5′-CCCAC-3′.

In certain embodiments, in a type V-A system, the 3′ end of the targeter stem sequence is linked by no more than 1, 2, 3, 4, 5, 6, 7, 8, 9, or 10 nucleotides to the 5′ end of the spacer sequence. In certain embodiments, the targeter stem sequence and the spacer sequence are adjacent to each other, directly linked by an internucleotide bond. In certain embodiments, the targeter stem sequence and the spacer sequence are linked by one nucleotide, e.g., a uridine. In certain embodiments, the targeter stem sequence and the spacer sequence are linked by two or more nucleotides. In certain embodiments, the targeter stem sequence and the spacer sequence are linked by 1, 2, 3, 4, 5, 6, 7, 8, 9, or 10 nucleotides.

In certain embodiments, the targeter nucleic acid further comprises an additional nucleotide sequence 5′ to the targeter stem sequence. In certain embodiments, the additional nucleotide sequence comprises at least 1 (e.g., at least 2, at least 3, at least 4, at least 5, at least 6, at least 7, at least 8, at least 9, at least 10, at least 15, at least 20, at least 25, at least 30, at least 35, at least 40, at least 45, or at least 50) nucleotides. In certain embodiments, the additional nucleotide sequence consists of 1, 2, 3, 4, 5, 6, 7, 8, 9, 10, 15, 20, 25, 30, 35, 40, 45, or 50 nucleotides. In certain embodiments, the additional nucleotide sequence consists of 2 nucleotides. In certain embodiments, the additional nucleotide sequence is reminiscent to the loop or a fragment thereof (e.g., one, two, three, or four nucleotides at the 3′ end of the loop) in a crRNA of a corresponding single guide CRISPR-Cas system. It is understood that an additional nucleotide sequence 5′ to the targeter stem sequence can be dispensable. Accordingly, in certain embodiments, the targeter nucleic acid does not comprise any additional nucleotide 5′ to the targeter stem sequence.

In certain embodiments, the targeter nucleic acid or the single guide nucleic acid further comprises an additional nucleotide sequence containing one or more nucleotides at the 3′ end that does not hybridize with the target nucleotide sequence. The additional nucleotide sequence may protect the targeter nucleic acid from degradation by 3′-5′ exonuclease. In certain embodiments, the additional nucleotide sequence is no more than 100 nucleotides in length. In certain embodiments, the additional nucleotide sequence is no more than 90, 80, 70, 60, 50, 40, 30, 20, or 10 nucleotides in length. In certain embodiments, the additional nucleotide sequence is at least 1, 2, 3, 4, 5, 6, 7, 8, 9, 10, 15, 20, 25, 30, 35, 40, 45, or 50 nucleotides in length. In certain embodiments, the additional nucleotide sequence is 5-100, 5-50, 5-40, 5-30, 5-25, 5-20, 5-15, 5-10, 10-100, 10-50, 10-40, 10-30, 10-25, 10-20, 10-15, 15-100, 15-50, 15-40, 15-30, 15-25, 15-20, 20-100, 20-50, 20-40, 20-30, 20-25, 25-100, 25-50, 25-40, 25-30, 30-100, 30-50, 30-40, 40-100, 40-50, or 50-100 nucleotides in length.

In certain embodiments, the additional nucleotide sequence forms a hairpin with the spacer sequence. Such secondary structure may increase the specificity of guide nucleic acid or the engineered, non-naturally occurring system (see, Kocak et al. (2019) Nat. Biotech. 37:657-66). In certain embodiments, the free energy change during the hairpin formation is greater than or equal to −20 kcal/mol, −15 kcal/mol, −14 kcal/mol, −13 kcal/mol, −12 kcal/mol, −11 kcal/mol, or −10 kcal/mol. In certain embodiments, the free energy change during the hairpin formation is greater than or equal to −5 kcal/mol, −6 kcal/mol, −7 kcal/mol, −8 kcal/mol, −9 kcal/mol, −10 kcal/mol, −11 kcal/mol, −12 kcal/mol, −13 kcal/mol, −14 kcal/mol, or −15 kcal/mol. In certain embodiments, the free energy change during the hairpin formation is in the range of −20 to −10 kcal/mol, −20 to −11 kcal/mol, −20 to −12 kcal/mol, −20 to −13 kcal/mol, −20 to −14 kcal/mol, −20 to −15 kcal/mol, −15 to −10 kcal/mol, −15 to −11 kcal/mol, −15 to −12 kcal/mol, −15 to −13 kcal/mol, −15 to −14 kcal/mol, −14 to −10 kcal/mol, −14 to −11 kcal/mol, −14 to −12 kcal/mol, −14 to −13 kcal/mol, −13 to −10 kcal/mol, −13 to −11 kcal/mol, −13 to −12 kcal/mol, −12 to −10 kcal/mol, −12 to −11 kcal/mol, or −11 to −10 kcal/mol. In other embodiments, the targeter nucleic acid or the single guide nucleic acid does not comprise any nucleotide 3′ to the spacer sequence.

In certain embodiments, the modulator nucleic acid further comprises an additional nucleotide sequence 3′ to the modulator stem sequence. In certain embodiments, the additional nucleotide sequence comprises at least 1 (e.g., at least 2, at least 3, at least 4, at least 5, at least 6, at least 7, at least 8, at least 9, at least 10, at least 15, at least 20, at least 25, at least 30, at least 35, at least 40, at least 45, or at least 50) nucleotides. In certain embodiments, the additional nucleotide sequence consists of 1, 2, 3, 4, 5, 6, 7, 8, 9, 10, 15, 20, 25, 30, 35, 40, 45, or 50 nucleotides. In certain embodiments, the additional nucleotide sequence consists of 1 nucleotide (e.g., uridine). In certain embodiments, the additional nucleotide sequence consists of 2 nucleotides. In certain embodiments, the additional nucleotide sequence is reminiscent to the loop or a fragment thereof (e.g., one, two, three, or four nucleotides at the 5′ end of the loop) in a crRNA of a corresponding single guide CRISPR-Cas system. It is understood that an additional nucleotide sequence 3′ to the modulator stem sequence can be dispensable. Accordingly, in certain embodiments, the modulator nucleic acid does not comprise any additional nucleotide 3″ to the modulator stem sequence.

It is understood that the additional nucleotide sequence 5′ to the targeter stem sequence and the additional nucleotide sequence 3′ to the modulator stem sequence, if present, may interact with each other. For example, although the nucleotide immediately 5′ to the targeter stem sequence and the nucleotide immediately 3′ to the modulator stem sequence do not form a Watson-Crick base pair (otherwise they would constitute part of the targeter stem sequence and part of the modulator stem sequence, respectively), other nucleotides in the additional nucleotide sequence 5′ to the targeter stem sequence and the additional nucleotide sequence 3′ to the modulator stem sequence may form one, two, three, or more base pairs (e.g., Watson-Crick base pairs). Such interaction may affect the stability of a complex comprising the targeter nucleic acid and the modulator nucleic acid.

The stability of a complex comprising a targeter nucleic acid and a modulator nucleic acid can be assessed by the Gibbs free energy change (AG) during the formation of the complex, cither calculated or actually measured. Where all the predicted base pairing in the complex occurs between a base in the targeter nucleic acid and a base in the modulator nucleic acid, i.e., there is no intra-strand secondary structure, the ΔG during the formation of the complex correlates generally with the ΔG during the formation of a secondary structure within the corresponding single guide nucleic acid. Methods of calculating or measuring the ΔG are known in the art. An exemplary method is RNAfold (rna.tbi.univie.ac.at/cgi-bin/RNA WebSuite/RNAfold.cgi) as disclosed in Gruber et al. (2008) Nucleic Acids Res., 36 (Web Server issue): W70-W74. Unless indicated otherwise, the ΔG values in the present disclosure are calculated by RNAfold for the formation of a secondary structure within a corresponding single guide nucleic acid. In certain embodiments, the ΔG is lower than or equal to −1 kcal/mol, e.g., lower than or equal to −2 kcal/mol, lower than or equal to −3 kcal/mol, lower than or equal to −4 kcal/mol, lower than or equal to −5 kcal/mol, lower than or equal to −6 kcal/mol, lower than or equal to −7 kcal/mol, lower than or equal to −7.5 kcal/mol, or lower than or equal to −8 kcal/mol. In certain embodiments, the ΔG is greater than or equal to −10 kcal/mol, e.g., greater than or equal to −9 kcal/mol, greater than or equal to −8.5 kcal/mol, or greater than or equal to −8 kcal/mol. In certain embodiments, the ΔG is in the range of-10 to −4 kcal/mol. In certain embodiments, the ΔG is in the range of −8 to −4 kcal/mol, −7 to −4 kcal/mol, −6 to −4 kcal/mol, −5 to −4 kcal/mol, −8 to −4.5 kcal/mol, −7 to −4.5 kcal/mol, −6 to −4.5 kcal/mol, or −5 to −4.5 kcal/mol. In certain embodiments, the ΔG is about −8 kcal/mol, −7 kcal/mol, −6 kcal/mol, −5 kcal/mol, −4.9 kcal/mol, −4.8 kcal/mol, −4.7 kcal/mol, −4.6 kcal/mol, −4.5 kcal/mol, −4.4 kcal/mol, −4.3 kcal/mol, −4.2 kcal/mol, −4.1 kcal/mol, or −4 kcal/mol.

It is understood that the ΔG may be affected by a sequence in the targeter nucleic acid that is not within the targeter stem sequence, and/or a sequence in the modulator nucleic acid that is not within the modulator stem sequence. For example, one or more base pairs (e.g., Watson-Crick base pair) between an additional sequence 5′ to the targeter stem sequence and an additional sequence 3′ to the modulator stem sequence may reduce the ΔG, i.e., stabilize the nucleic acid complex. In certain embodiments, the nucleotide immediately 5′ to the targeter stem sequence comprises a uracil or is a uridine, and the nucleotide immediately 3′ to the modulator stem sequence comprises a uracil or is a uridine, thereby forming a nonconventional U-U base pair.

In certain embodiments, the modulator nucleic acid or the single guide nucleic acid comprises a nucleotide sequence referred to herein as a “5′ tail” positioned 5′ to the modulator stem sequence. In a naturally occurring type V-A CRISPR-Cas system, the 5′ tail is a nucleotide sequence positioned 5′ to the stem-loop structure of the crRNA. A 5′ tail in an engineered type V-A CRISPR-Cas system, whether single guide or dual guide, can be reminiscent to the 5′ tail in a corresponding naturally occurring type V-A CRISPR-Cas system.

Without being bound by theory, it is contemplated that the 5′ tail may participate in the formation of the CRISPR-Cas complex. For example, in certain embodiments, the 5′ tail forms a pseudoknot structure with the modulator stem sequence, which is recognized by the Cas protein (see, Yamano et al. (2016) Cell, 165:949). In certain embodiments, the 5′ tail is at least 3 (e.g., at least 4 or at least 5) nucleotides in length. In certain embodiments, the 5′ tail is 3, 4, or 5 nucleotides in length. In certain embodiments, the nucleotide at the 3′ end of the 5′ tail comprises a uracil or is a uridine. In certain embodiments, the second nucleotide in the 5′ tail, the position counted from the 3′ end, comprises a uracil or is a uridine. In certain embodiments, the third nucleotide in the 5′ tail, the position counted from the 3′ end, comprises an adenine or is an adenosine. This third nucleotide may form a base pair (e.g., a Watson-Crick base pair) with a nucleotide 5′ to the modulator stem sequence. Accordingly, in certain embodiments, the modulator nucleic acid comprises a uridine or a uracil-containing nucleotide 5′ to the modulator stem sequence. In certain embodiments, the 5′ tail comprises the nucleotide sequence of 5′-AUU-3′. In certain embodiments, the 5′ tail comprises the nucleotide sequence of 5′-AAUU-3″. In certain embodiments, the 5′ tail comprises the nucleotide sequence of 5′-UAAUU-3′. In certain embodiments, the 5′ tail is positioned immediately 5′ to the modulator stem sequence.

In certain embodiments, the single guide nucleic acid, the targeter nucleic acid, and/or the modulator nucleic acid are designed to reduce the degree of secondary structure other than the hybridization between the targeter stem sequence and the modulator stem sequence. In certain embodiments, no more than about 75%, 50%, 40%, 30%, 25%, 20%, 15%, 10%, 5%, 1%, or fewer of the nucleotides of the single guide nucleic acid other than the targeter stem sequence and the modulator stem sequence participate in self-complementary base pairing when optimally folded. In certain embodiments, no more than about 75%, 50%, 40%, 30%, 25%, 20%, 15%, 10%, 5%, 1%, or fewer of the nucleotides of the targeter nucleic acid and/or the modulator nucleic acid participate in self-complementary base pairing when optimally folded. Optimal folding may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see e.g., A. R. Gruber et al., 2008, Cell 106 (1): 23-24; and PA Carr and GM Church, 2009, Nature Biotechnology 27 (12): 1151-62).

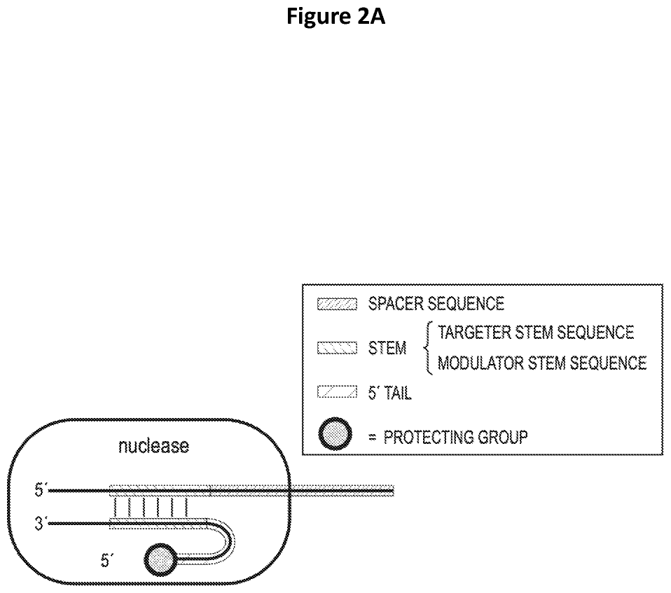

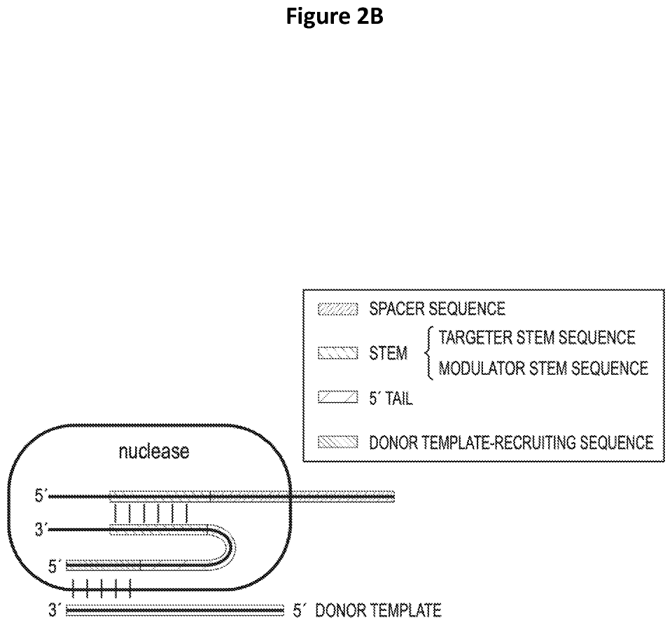

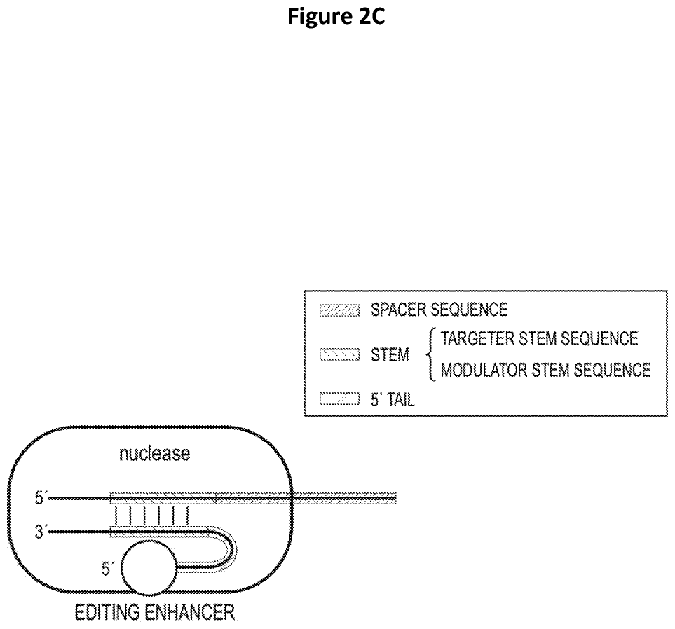

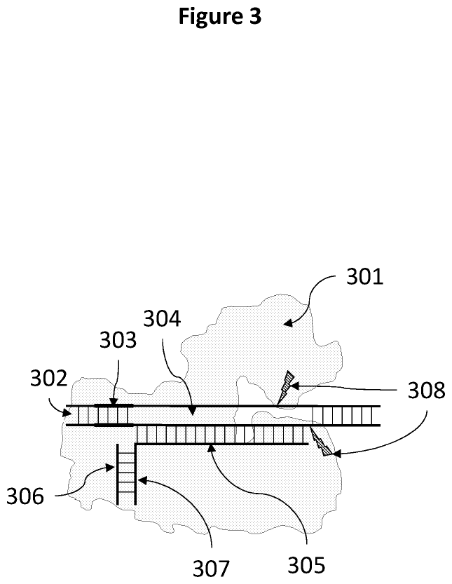

The targeter nucleic acid is directed to a specific target nucleotide sequence, and a donor template can be designed to modify the target nucleotide sequence or a sequence nearby. It is understood, therefore, that association of the single guide nucleic acid, the targeter nucleic acid, or the modulator nucleic acid with a donor template can increase editing efficiency and reduce off-targeting. Accordingly, in certain embodiments, the single guide nucleic acid or the modulator nucleic acid further comprises a donor template-recruiting sequence capable of hybridizing with a donor template (see FIG. 2B). Donor templates are described in the “Donor Templates” subsection of section II infra. The donor template and donor template-recruiting sequence can be designed such that they bear sequence complementarity. In certain embodiments, the donor template-recruiting sequence is at least 90% (e.g., at least 91%, at least 92%, at least 93%, at least 94%, at least 95%, at least 96%, at least 97%, at least 98%, or at least 99%) complementary to at least a portion of the donor template. In certain embodiments, the donor template-recruiting sequence is 100% complementary to at least a portion of the donor template. In certain embodiments, where the donor template comprises an engineered sequence not homologous to the sequence to be repaired, the donor template-recruiting sequence is capable of hybridizing with the engineered sequence in the donor template. In certain embodiments, the donor template-recruiting sequence is at least 20, 25, 30, 35, 40, 45, 50, 55, 60, 65, 70, 75, 80, 85, 90, 95, or 100 nucleotides in length. In certain embodiments, the donor template-recruiting sequence is positioned at or near the 5′ end of the single guide nucleic acid or at or near the 5′ end of the modulator nucleic acid. In certain embodiments, the donor template-recruiting sequence is linked to the 5′ tail, if present, or to the modulator stem sequence, of the single guide nucleic acid or the modulator nucleic acid through an internucleotide bond or a nucleotide linker.

In certain embodiments, the single guide nucleic acid or the modulator nucleic acid further comprises an editing enhancer sequence, which increases the efficiency of gene editing and/or homology-directed repair (HDR) (see FIG. 2C). Exemplary editing enhancer sequences are described in Park et al. (2018) Nat. Commun. 9:3313. In certain embodiments, the editing enhancer sequence is positioned 5′ to the 5′ tail, if present, or 5′ to the single guide nucleic acid or the modulator stem sequence. In certain embodiments, the editing enhancer sequence is 1-50, 4-50, 9-50, 15-50, 25-50, 1-25, 4-25, 9-25, 15-25, 1-15, 4-15, 9-15, 1-9, 4-9, or 1-4 nucleotides in length. In certain embodiments, the editing enhancer sequence is about 1, 2, 3, 4, 5, 6, 7, 8, 9, 10, 15, 20, 25, 30, 35, 40, 45, 50, or 55 nucleotides in length. The editing enhancer sequence is designed to minimize homology to the target nucleotide sequence or any other sequence that the engineered, non-naturally occurring system may be contacted to, e.g., the genome sequence of a cell into which the engineered, non-naturally occurring system is delivered. In certain embodiments, the editing enhancer is designed to minimize the presence of hairpin structure. The editing enhancer can comprise one or more of the chemical modifications disclosed herein.

The single guide nucleic acid, the modulator nucleic acid, and/or the targeter nucleic acid can further comprise a protective nucleotide sequence that prevents or reduces nucleic acid degradation. In certain embodiments, the protective nucleotide sequence is at least 5 (e.g., at least 10, at least 15, at least 20, at least 25, at least 30, at least 35, at least 40, at least 45, or at least 50) nucleotides in length. The length of the protective nucleotide sequence increases the time for an exonuclease to reach the 5′ tail, modulator stem sequence, targeter stem sequence, and/or spacer sequence, thereby protecting these portions of the single guide nucleic acid, the modulator nucleic acid, and/or the targeter nucleic acid from degradation by an exonuclease. In certain embodiments, the protective nucleotide sequence forms a secondary structure, such as a hairpin or a tRNA structure, to reduce the speed of degradation by an exonuclease (see, for example, Wu et al. (2018) Cell. Mol. Life Sci., 75 (19): 3593-3607). Secondary structures can be predicted by methods known in the art, such as the online webserver RNAfold developed at University of Vienna using the centroid structure prediction algorithm (see, Gruber et al. (2008) Nucleic Acids Res., 36: W70). Certain chemical modifications, which may be present in the protective nucleotide sequence, can also prevent or reduce nucleic acid degradation, as disclosed in the “RNA Modifications” subsection infra.

A protective nucleotide sequence is typically located at the 5′ or 3′ end of the single guide nucleic acid, the modulator nucleic acid, and/or the targeter nucleic acid. In certain embodiments, the single guide nucleic acid comprises a protective nucleotide sequence at the 5′ end, at the 3′ end, or at both ends, optionally through a nucleotide linker. In certain embodiments, the modulator nucleic acid comprises a protective nucleotide sequence at the 5′ end, at the 3′ end, or at both ends, optionally through a nucleotide linker. In particular embodiments, the modulator nucleic acid comprises a protective nucleotide sequence at the 5′ end (see FIG. 2A). In certain embodiments, the targeter nucleic acid comprises a protective nucleotide sequence at the 5′ end, at the 3′ end, or at both ends, optionally through a nucleotide linker.

As described above, various nucleotide sequences can be present in the 5′ portion of a single nucleic acid or a modulator nucleic acid, including but not limited to a donor template-recruiting sequence, an editing enhancer sequence, a protective nucleotide sequence, and a linker connecting such sequence to the 5′ tail, if present, or to the modulator stem sequence. It is understood that the functions of donor template recruitment, editing enhancement, protection against degradation, and linkage are not exclusive to each other, and one nucleotide sequence can have one or more of such functions. For example, in certain embodiments, the single guide nucleic acid or the modulator nucleic acid comprises a nucleotide sequence that is both a donor template-recruiting sequence and an editing enhancer sequence. In certain embodiments, the single guide nucleic acid or the modulator nucleic acid comprises a nucleotide sequence that is both a donor template-recruiting sequence and a protective sequence. In certain embodiments, the single guide nucleic acid or the modulator nucleic acid comprises a nucleotide sequence that is both an editing enhancer sequence and a protective sequence. In certain embodiments, the single guide nucleic acid or the modulator nucleic acid comprises a nucleotide sequence that is a donor template-recruiting sequence, an editing enhancer sequence, and a protective sequence. In certain embodiments, the nucleotide sequence 5′ to the 5′ tail, if present, or 5′ to the modulator stem sequence is 1-90, 1-80, 1-70, 1-60, 1-50, 1-40, 1-30, 1-20, 1-10, 10-90, 10-80, 10-70, 10-60, 10-50, 10-40, 10-30, 10-20, 20-90, 20-80, 20-70, 20-60, 20-50, 20-40, 20-30, 30-90, 30-80, 30-70, 30-60, 30-50, 30-40, 40-90, 40-80, 40-70, 40-60, 40-50, 50-90, 50-80, 50-70, 50-60, 60-90, 60-80, 60-70, 70-90, 70-80, or 80-90 nucleotides in length.

In certain embodiments, an engineered, non-naturally occurring system further comprises one or more compounds (e.g., small molecule compounds) that enhance HDR and/or inhibit NHEJ. Exemplary compounds having such functions are described in Maruyama et al. (2015) Nat Biotechnol. 33 (5): 538-42: Chu et al. (2015) Nat Biotechnol. 33 (5): 543-48; Yu et al. (2015) Cell Stem Cell 16 (2): 142-47: Pinder et al. (2015) Nucleic Acids Res. 43 (19): 9379-92; and Yagiz et al. (2019) Commun. Biol. 2:198. In certain embodiments, an engineered, non-naturally occurring system further comprises one or more compounds selected from the group consisting of DNA ligase IV antagonists (e.g., SCR7 compound, Ad4 E1B55K protein, and Ad4 E4orf6 protein), RAD51 agonists (e.g., RS-1), DNA-dependent protein kinase (DNA-PK) antagonists (e.g., NU7441 and KU0060648), B3-adrenergic receptor agonists (e.g., L755507), inhibitors of intracellular protein transport from the ER to the Golgi apparatus (e.g., brefeldin A), and any combinations thereof.

In certain embodiments, an engineered, non-naturally occurring system comprising a targeter nucleic acid and a modulator nucleic acid is tunable or inducible. For example, in certain embodiments, the targeter nucleic acid, the modulator nucleic acid, and/or the Cas protein can be introduced to the target nucleotide sequence at different times, the system becoming active only when all components are present. In certain embodiments, the amounts of the targeter nucleic acid, the modulator nucleic acid, and/or the Cas protein can be titrated to achieve desired efficiency and specificity. In certain embodiments, excess amount of a nucleic acid comprising the targeter stem sequence or the modulator stem sequence can be added to the system, thereby dissociating the complex of the targeter nucleic and modulator nucleic acid and turning off the system.

### C. gNA Modifications

Guide nucleic acids, including a single guide nucleic acid, a targeter nucleic acid, and/or a modulator nucleic acid, may comprise a DNA (e.g., modified DNA), an RNA (e.g., modified RNA), or a combination thereof. In certain embodiments, the single guide nucleic acid comprises a DNA (e.g., modified DNA), an RNA (e.g., modified RNA), or a combination thereof. In certain embodiments, the targeter nucleic acid comprises a DNA (e.g., modified DNA), an RNA (e.g., modified RNA), or a combination thereof. In certain embodiments, the modulator nucleic acid comprises a DNA (e.g., modified DNA), an RNA (e.g., modified RNA), or a combination thereof. Spacer sequences can be presented as DNA sequences by including thymidines (T) rather than uridines (U). It is understood that corresponding RNA sequences and DNA/RNA chimeric sequences are also contemplated. For example, where the spacer sequence is an RNA, its sequence can be derived from a DNA sequence disclosed herein by replacing each T with U. As a result, for the purpose of describing a nucleotide sequence, T and U are used interchangeably herein.

In certain embodiments engineered, non-naturally occurring systems comprising a targeter nucleic acid comprising: a spacer sequence designed to hybridize with a target nucleotide sequence and a targeter stem sequence; and a modulator nucleic acid comprising a modulator stem sequence complementary to the targeter stem sequence, and, optionally, a 5′ sequence, e.g., a tail sequence, wherein, in a single guide nucleic acid the targeter nucleic acid and the modulator nucleic acid are part of a single polynucleotide, and in a dual guide nucleic acid, the targeter nucleic acid and the modulator nucleic acid are separate nucleic acids: modifications can include one or more chemical modifications to one or more nucleotides or internucleotide linkages at or near the 3′ end of the targeter nucleic acid (dual and single gNA), at or near the 5′ end of the targeter nucleic acid (dual gNA), at or near the 3′ end of the modulator nucleic acid (dual gNA), at or near the 5′ end of the modulator nucleic acid (single and dual gNA), or combinations thereof as appropriate for single or dual gNA. In certain embodiments, the Cas nuclease is a type V-A Cas nuclease. Modulator and/or targeter nucleic sequences can include further sequences, as detailed in the Guide Nucleic Acids section, and modifications can be in these further sequences, as appropriate and apparent to one of skill in the art. In embodiments described in this section, below, in certain embodiments, guide nucleic acid is oriented from 5′ at the modulator nucleic acid to 3′ at the modulator stem sequence, and 5′ at the targeter stem sequence to 3′ at the targeter sequence (see, e.g., FIGS. 1A and 1B): in certain embodiments, as appropriate, guide nucleic acid is oriented from 3′ at the modulator nucleic acid to 5′ at the modulator stem sequence, and 3′ at the targeter stem sequence to 5′ at the targeter sequence.

The targeter nucleic acid may comprise a DNA (e.g., modified DNA), an RNA (e.g., modified RNA), or a combination thereof. The modulator nucleic acid may comprise a DNA (e.g., modified DNA), an RNA (e.g., modified RNA), or a combination thereof. In certain embodiments, the targeter nucleic acid is an RNA and the modulator nucleic acid is an RNA. A targeter nucleic acid in the form of an RNA is also called targeter RNA, and a modulator nucleic acid in the form of an RNA is also called modulator RNA. The nucleotide sequences disclosed herein are presented as DNA sequences by including thymidines (T) and/or RNA sequences including uridines (U). It is understood that corresponding DNA sequences, RNA sequences, and DNA/RNA chimeric sequences are also contemplated. For example, where a spacer sequence is presented as a DNA sequence, a nucleic acid comprising this spacer sequence as an RNA can be derived from the DNA sequence disclosed herein by replacing each T with U. As a result, for the purpose of describing a nucleotide sequence, T and U are used interchangeably herein.

In certain embodiments some or all of the gNA is RNA, e.g., a gRNA. In certain embodiments, 5-100%, 10-100%, 20-100%, 30-100%, 40-100%, 50-100%, 60-100%, 70-100%, 80-100%, 90-100%, 95-100%, 99-100%, 99.5-100% of the gNA is gRNA. In certain embodiments, 20%-80%, 20%-70%, 20%-60%, 20%-50%, 20%-40%, 20%-30%, 30%-80%, 30%-70%, 30%-60%, 30%-50%, 30%-40%, 40%-80%, 40%-70%, 40%-60%, 40%-50%, 50%-80%, 50%-70%, 50%-60%, 60%-80%, 60%-70%, or 70%-80% of gNA is RNA. In certain embodiments, 50% of the gNA is RNA. In certain embodiments, 70% of the gNA is RNA. In certain embodiments, 90% of the gNA is RNA. In certain embodiments, 100% of the gNA is RNA, e.g., a gRNA. In further embodiments, the remaining portion of the gNA that is not RNA comprises a modified ribonucleotide, a deoxyribonucleotide, a modified deoxyribonucleotide, or a synthetic, e.g., unnatural nucleotide, for example, not intended to be limiting, threose nucleic acid, locked nucleic acid, peptide nucleic acid, arabinonucleic acid, hexose nucleic acid, among others.

In certain embodiments, the targeter nucleic acid and/or the modulator nucleic acid are RNAs with one or more modifications in a ribose group, one or more modifications in a phosphate group, one or more modifications in a nucleobase, one or more terminal modifications, or a combination thereof. Exemplary modifications are disclosed in U.S. Pat. Nos. 10,900,034 and 10,767,175, U.S. Patent Application Publication No. 2018/0119140, Watts et al. (2008) Drug Discov. Today 13:842-55, and Hendel et al. (2015) NAT. BIOTECHNOL. 33:985.

In certain embodiments, a targeter nucleic acid, e.g., RNA, comprises at least one nucleotide at or near the 3′ end comprising a modification to a ribose, phosphate group, nucleobase, or terminal modification. In certain embodiments, the 3′ end of the targeter nucleic acid comprises the spacer sequence. In certain embodiments, the 3′ end of the targeter nucleic acid comprises the targeter stem sequence. Exemplary modifications are disclosed in Dang et al. (2015) Genome Biol. 16:280. Kocaz et al. (2019) Nature Biotech. 37:657-66, Liu et al. (2019) Nucleic Acids Res. 47 (8): 4169-4180. Schubert et al. (2018) J. Cytokine Biol. 3 (1): 121. Tong et al. (2019) Genome Biol. 20 (1): 15. Watts et al. (2008) Drug Discov. Today 13 (19-20): 842-55, and Wu et al. (2018) Cell Mol. Life. Sci. 75 (19): 3593-607.

Modifications in a ribose group include but are not limited to modifications at the 2′ position or modifications at the 4′ position. For example, in certain embodiments, the ribose comprises 2′-O—C1-4alkyl, such as 2′-O-methyl (2′-OMe, or M). In certain embodiments, the ribose comprises 2′-O—C1-3alkyl-O-C1-3alkyl, such as 2′-methoxyethoxy (2′-O—CH₂CH₂OCH₃) also known as 2′-O-(2-methoxyethyl) or 2′-MOE. In certain embodiments, the ribose comprises 2′-O-allyl. In certain embodiments, the ribose comprises 2′-O-2,4-Dinitrophenol (DNP). In certain embodiments, the ribose comprises 2′-halo, such as 2′-F, 2′-Br, 2′-Cl, or 2′-I. In certain embodiments, the ribose comprises 2′—NH₂. In certain embodiments, the ribose comprises 2′-H (e.g., a deoxynucleotide). In certain embodiments, the ribose comprises 2′-arabino or 2′-F-arabino. In certain embodiments, the ribose comprises 2′-LNA or 2′-ULNA. In certain embodiments, the ribose comprises a 4′-thioribosyl.

Modifications can also include a deoxy group, for example a 2′-deoxy-3′-phosphonoacetate (DP), a 2′-deoxy-3′-thiophosphonoacetate (DSP).

Internucleotide linkage modifications in a phosphate group include but are not limited to a phosphorothioate(S), a chiral phosphorothioate, a phosphorodithioate, a boranophosphonatc. a C₁₋₄alkyl phosphonate such as a methylphosphonate, a boranophosphonate, a phosphonocarboxylate such as a phosphonoacetate (P), a phosphonocarboxylate ester such as a phosphonoacetate ester, an amide, a thiophosphonocarboxylate such as a thiophosphonoacetate (SP), a thiophosphonocarboxylate ester such as a thiophosphonoacetate ester, and a 2′,5′-linkage having a phosphodiester or any of the modified phosphates above. Various salts, mixed salts and free acid forms are also included.

Modifications in a nucleobase include but are not limited to 2-thiouracil, 2-thiocytosine, 4-thiouracil, 6-thioguanine, 2-aminoadenine, 2-aminopurine, pseudouracil, hypoxanthine, 7-deazaguanine, 7-deaza-8-azaguanine, 7-deazaadenine, 7-deaza-8-azaadenine, 5-methylcytosine, 5-methyluracil, 5-hydroxymethylcytosine, 5-hydroxymethyluracil, 5,6-dehydrouracil, 5-propynylcytosine, 5-propynyluracil, 5-ethynyleytosine, 5-ethynyluracil, 5-allyluracil, 5-allylcytosine, 5-aminoallyluracil, 5-aminoallyl-cytosine, 5-bromouracil, 5-iodouracil, diaminopurine, difluorotoluene, dihydrouracil, an abasic nucleotide, Z base, P base, Unstructured Nucleic Acid, isoguanine, isocytosine (see. Piccirilli et al. (1990) NATURE. 343: 33), 5-methyl-2-pyrimidine (see, Rappaport (1993) BIOCHEMISTRY, 32:3047), x(A,G,C,T), and y(A,G,C,T).

Terminal modifications include but are not limited to polyethyleneglycol (PEG), hydrocarbon linkers (such as heteroatom (O,S,N)-substituted hydrocarbon spacers; halo-substituted hydrocarbon spacers; keto-, carboxyl-, amido-, thionyl-, carbamoyl-, thionocarbamaoyl-containing hydrocarbon spacers, propanediol), spermine linkers, dyes such as fluorescent dyes (for example, fluoresceins, rhodamines, cyanines), quenchers (for example, dabcyl, BHQ), and other labels (for example biotin, digoxigenin, acridine, streptavidin, avidin, peptides and/or proteins). In certain embodiments, a terminal modification comprises a conjugation (or ligation) of the RNA to another molecule comprising an oligonucleotide (such as deoxyribonucleotides and/or ribonucleotides), a peptide, a protein, a sugar, an oligosaccharide, a steroid, a lipid, a folic acid, a vitamin and/or other molecule. In certain embodiments, a terminal modification incorporated into the RNA is located internally in the RNA sequence via a linker such as 2-(4-butylamidofluorescein) propane-1.3-diol bis (phosphodiester) linker, which is incorporated as a phosphodiester linkage and can be incorporated anywhere between two nucleotides in the RNA.

The modifications disclosed above can be combined in the targeter nucleic acid and/or the modulator nucleic acid that are in the form of RNA. In certain embodiments, the modification in the RNA is selected from the group consisting of incorporation of 2′-O-methyl-3′phosphorothioate (MS), 2′-O-methyl-3′-phosphonoacetate (MP), 2′-O-methyl-3′-thiophosphonoacetate (MSP), 2′-halo-3′-phosphorothioate (e.g., 2′-fluoro-3′-phosphorothioate), 2′-halo-3′-phosphonoacetate (e.g., 2′-fluoro-3′-phosphonoacetate), and 2′-halo-3′-thiophosphonoacetate (e.g., 2′-fluoro-3′-thiophosphonoacetate).

In certain embodiments, modifications can include 2′-O-methyl (M), a phosphorothioate(S), a phosphonoacetate (P), a thiophosphonoacetate (SP), a 2′-O-methyl-3′-phosphorothioate (MS), a 2′-O-methyl-3′-phosphonoacetate (MP), a 2′-O-methyl-3′-thiophosphonoacetate (MSP), a 2′-deoxy-3′-phosphonoacetate (DP), a 2′-deoxy-3′-thiophosphonoacetate (DSP), or a combination thereof, at or near either the 3′ or 5′ end of either the targeter or modulator nucleic acid, as appropriate for single or dual gNA. In certain embodiments, modifications can include either a 5′ or a 3′ propanediol or C3 linker modification.

In certain embodiments, the modification alters the stability of the RNA. In certain embodiments, the modification enhances the stability of the RNA, e.g., by increasing nuclease resistance of the RNA relative to a corresponding RNA without the modification. Stability-enhancing modifications include but are not limited to incorporation of 2′-O-methyl, a 2′-O—C₁-alkyl, 2′-halo (e.g., 2′-F, 2′-Br, 2′-Cl, or 2′-I), 2′MOE, a 2′-O—C₁₋₃alkyl-O—C₁₋₃alkyl, 2′—NH₂, 2′-H (or 2′-deoxy), 2′-arabino, 2′-F-arabino, 4′-thioribosyl sugar moiety, 3′-phosphorothioate, 3′-phosphonoacetate, 3′-thiophosphonoacetate, 3′-methylphosphonate, 3′-boranophosphate, 3′-phosphorodithioate, locked nucleic acid (“LNA”) nucleotide which comprises a methylene bridge between the 2′ and 4′ carbons of the ribose ring, and unlocked nucleic acid (“ULNA”) nucleotide. Such modifications are suitable for use as a protecting group to prevent or reduce degradation of the 5′ sequence, e.g., a tail sequence, modulator stem sequence (dual guide nucleic acids), targeter stem sequence (dual guide nucleic acids), and/or spacer sequence (see, the “Targeter and Modulator nucleic acids” subsection).

In certain embodiments, the modification alters the specificity of the engineered, non-naturally occurring system. In certain embodiments, the modification enhances the specificity of the engineered, non-naturally occurring system, e.g., by enhancing on-target binding and/or cleavage, or reducing off-target binding and/or cleavage, or a combination thereof. Specificity-enhancing modifications include but are not limited to 2-thiouracil, 2-thiocytosine, 4-thiouracil, 6-thioguanine, 2-aminoadenine, and pseudouracil. Within 10, 5, 4, 3, 2, or 1 nucleotide of the 3″ end, for example the 3′ end nucleotide, is modified

In certain embodiments, the modification alters the immunostimulatory effect of the RNA relative to a corresponding RNA without the modification. For example, in certain embodiments, the modification reduces the ability of the RNA to activate TLR7, TLR8, TLR9, TLR3, RIG-I, and/or MDA5.

In certain embodiments, the targeter nucleic acid and/or the modulator nucleic acid comprise at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 modified nucleotides or internucleotide linkages. The modification can be made at one or more positions in the targeter nucleic acid and/or the modulator nucleic acid such that these nucleic acids retain functionality. For example, the modified nucleic acids can still direct the Cas protein to the target nucleotide sequence and allow the Cas protein to exert its effector function. It is understood that the particular modification(s) at a position may be selected based on the functionality of the nucleotide or internucleotide linkage at the position. For example, a specificity-enhancing modification may be suitable for a nucleotide or internucleotide linkage in the spacer sequence, the targeter stem sequence, or the modulator stem sequence. A stability-enhancing modification may be suitable for one or more terminal nucleotides or internucleotide linkages in the targeter nucleic acid and/or the modulator nucleic acid. In certain embodiments, at least 1 (e.g., at least 2, at least 3, at least 4, or at least 5) terminal nucleotides or internucleotide linkages at or near the 5′ end and/or at least 1 (e.g., at least 2, at least 3, at least 4, or at least 5) terminal nucleotides or internucleotide linkages at or near the 3′ end of the targeter nucleic acid are modified. In certain embodiments, 5 or fewer (e.g., 1 or fewer, 2 or fewer, 3 or fewer, or 4 or fewer) terminal nucleotides or internucleotide linkages at or near the 5′ end and/or 5 or fewer (e.g., 1 or fewer, 2 or fewer, 3 or fewer, or 4 or fewer) terminal nucleotides or internucleotide linkages at or near the 3′ end of the targeter nucleic acid are modified. In certain embodiments, at least 1 (e.g., at least 2, at least 3, at least 4, or at least 5) terminal nucleotides or internucleotide linkages at or near the 5′ end and/or at least 1 (e.g., at least 2, at least 3, at least 4, or at least 5) terminal nucleotides or internucleotide linkages at or near the 3′ end of the modulator nucleic acid are modified. In certain embodiments, 5 or fewer (e.g., 1 or fewer, 2 or fewer, 3 or fewer, or 4 or fewer) terminal nucleotides or internucleotide linkages at or near the 5′ end and/or 5 or fewer (e.g., 1 or fewer, 2 or fewer, 3 or fewer, or 4 or fewer) terminal nucleotides or internucleotide linkages at or near the 3′ end of the modulator nucleic acid are modified. Selection of positions for modifications is described in U.S. Pat. Nos. 10,900,034 and 10,767,175. As used in this paragraph, where the targeter or modulator nucleic acid is a combination of DNA and RNA, the nucleic acid as a whole is considered as an RNA, and the DNA nucleotide(s) are considered as modification(s) of the RNA, including a 2′-H modification of the ribose and optionally a modification of the nucleobase.

It is understood that, in dual guide nucleic acid systems the targeter nucleic acid and the modulator nucleic acid, while not in the same nucleic acids, i.e., not linked end-to-end through a traditional internucleotide bond, can be covalently conjugated to each other through one or more chemical modifications introduced into these nucleic acids, thereby increasing the stability of the double-stranded complex and/or improving other characteristics of the system.

IV. Composition and Methods for Targeting, Editing, and/or Modifying Genomic DNA

An engineered, non-naturally occurring system, such as disclosed herein, can be useful for targeting, editing, and/or modifying a target nucleic acid, such as a DNA (e.g., genomic DNA) in a cell or organism.

The present invention provides a method of cleaving a target nucleic acid (e.g., DNA) comprising the sequence of a preselected target sequence or a portion thereof, the method comprising contacting the target DNA with an engineered, non-naturally occurring system disclosed herein, thereby resulting in cleavage of the target DNA.

In addition, the present invention provides a method of binding a target nucleic acid (e.g., DNA) comprising the sequence of a preselected target sequence or a portion thereof, the method comprising contacting the target DNA with an engineered, non-naturally occurring system disclosed herein, thereby resulting in binding of the system to the target DNA. This method can be useful, e.g., for detecting the presence and/or location of the a preselected target gene, for example, if a component of the system (e.g., the Cas protein) comprises a detectable marker.

In addition, provided are methods of modifying a target nucleic acid (e.g., DNA) comprising the sequence of a preselected target sequence or a portion thereof, or a structure (e.g., protein) associated with the target DNA (e.g., a histone protein in a chromosome), the method comprising contacting the target DNA with an engineered, non-naturally occurring system disclosed herein, wherein the Cas protein comprises an effector domain or is associated with an effector protein, thereby resulting in modification of the target DNA or the structure associated with the target DNA. The modification corresponds to the function of the effector domain or effector protein. Exemplary functions described in the “Cas Proteins” subsection in Section I supra are applicable hereto.

An engineered, non-naturally occurring system can be contacted with the target nucleic acid as a complex. Accordingly, in certain embodiments, a method comprises contacting the target nucleic acid with a CRISPR-Cas complex comprising a targeter nucleic acid, a modulator nucleic acid, and a Cas protein disclosed herein. In certain embodiments, the Cas protein is a type V-A, type V-C, or type V-D Cas protein (e.g., Cas nuclease). In certain embodiments, the Cas protein is a type V-A Cas protein (e.g., Cas nuclease).

In certain embodiments, provided is a method of editing a human genomic sequence at one of a group of preselected target gene loci, the method comprising delivering an engineered, non-naturally occurring system disclosed herein into a human cell, thereby resulting in editing of the genomic sequence at the target gene locus in the human cell. In certain embodiments, provided herein is a method of detecting a human genomic sequence at one of a group of preselected target gene loci, the method comprising delivering the engineered, non-naturally occurring system disclosed herein into a human cell, wherein a component of the system (e.g., the Cas protein) comprises a detectable marker, thereby detecting the target gene locus in the human cell. In certain embodiments, provided herein is a method of modifying a human chromosome at one of a group of preselected target gene loci, the method comprising delivering the engineered, non-naturally occurring system disclosed herein into a human cell, wherein the Cas protein comprises an effector domain or is associated with an effector protein, thereby resulting in modification of the chromosome at the target gene locus in the human cell.

The CRISPR-Cas complex may be delivered to a cell by introducing a pre-formed ribonucleoprotein (RNP) complex into the cell. Alternatively, one or more components of the CRISPR-Cas complex may be expressed in the cell. Exemplary methods of delivery are known in the art and described in, for example, U.S. Pat. Nos. 8,697,359, 10,113,167, 10,570,418, 10,829,787, 11,118,194, and 11,125,739 and U.S. Patent Application Publication Nos. 2015/0344912. 2018/0119140, and 2018/0282763.

It is understood that contacting a DNA (e.g., genomic DNA) in a cell with a CRISPR-Cas complex does not require delivery of all components of the complex into the cell. For example, one or more of the components may be pre-existing in the cell. In certain embodiments, the cell (or a parental/ancestral cell thereof) has been engineered to express the Cas protein, and the single guide nucleic acid (or a nucleic acid comprising a regulatory element operably linked to a nucleotide sequence encoding the single guide nucleic acid), the targeter nucleic acid (or a nucleic acid comprising a regulatory element operably linked to a nucleotide sequence encoding the targeter nucleic acid), and/or the modulator nucleic acid (or a nucleic acid comprising a regulatory element operably linked to a nucleotide sequence encoding the modulator nucleic acid) are delivered into the cell. In certain embodiments, the cell (or a parental/ancestral cell thereof) has been engineered to express the modulator nucleic acid, and the Cas protein (or a nucleic acid comprising a regulatory element operably linked to a nucleotide sequence encoding the Cas protein) and the targeter nucleic acid (or a nucleic acid comprising a regulatory element operably linked to a nucleotide sequence encoding the targeter nucleic acid) are delivered into the cell. In certain embodiments, the cell (or a parental/ancestral cell thereof) has been engineered to express the Cas protein and the modulator nucleic acid, and the targeter nucleic acid (or a nucleic acid comprising a regulatory element operably linked to a nucleotide sequence encoding the targeter nucleic acid) is delivered into the cell.

In certain embodiments, the target DNA is in the genome of a target cell. Accordingly, the present invention also provides a cell comprising the non-naturally occurring system or a CRISPR expression system described herein. In addition, the present invention provides a cell whose genome has been modified by the CRISPR-Cas system or complex disclosed herein.

The target cells can be mitotic or post-mitotic cells from any organism, such as a bacterial cell (e.g., E coli), an archaeal cell, a cell of a single-cell eukaryotic organism, a plant cell, an algal cell, e.g., Botryococcus braunii, Chlamydomonas reinhardtii, Nannochloropsis gaditana, Chlorella pyrenoidosa, Sargassum patens C. Agardh, or the like, a fungal cell (e.g., a yeast cell, such as S, cervisiae), an animal cell, a cell from an invertebrate animal (e.g. fruit fly, enidarian, echinoderm, nematode, etc.), a cell from a vertebrate animal (e.g., fish, amphibian, reptile, bird, mammal), a cell from a mammal, a cell from a rodent, or a cell from a human. The types of target cells include but are not limited to a stem cell (e.g., an embryonic stem (ES) cell, an induced pluripotent stem (iPS) cell, a germ cell), a somatic cell (e.g., a fibroblast, a hematopoietic cell, a T lymphocyte (e.g., CD8+ T lymphocyte), an NK cell, a neuron, a muscle cell, a bone cell, a hepatocyte, a pancreatic cell), an in vitro or in vivo embryonic cell of an embryo at any stage (e.g., a 1-cell, 2-cell, 4-cell, 8-cell: stage zebrafish embryo). Cells may be from established cell lines or may be primary cells (i.e., cells and cells cultures that have been derived from a subject and allowed to grow in vitro for a limited number of passages of the culture). For example, primary cultures are cultures that may have been passaged within 0 times, 1 time, 2 times, 4 times, 5 times, 10 times, or 15 times, but not enough times to go through the crisis stage. Typically, the primary cell lines are maintained for fewer than 10 passages in vitro. If the cells are primary cells, they may be harvest from an individual by any suitable method. For example, leukocytes may be harvested by apheresis, leukocytapheresis, or density gradient separation, while cells from tissues such as skin, muscle, bone marrow, spleen, liver, pancreas, lung, intestine, or stomach can be harvested by biopsy. The harvested cells may be used immediately, or may be stored under frozen conditions with a cryopreservative and thawed at a later time in a manner as commonly known in the art.

### A. Ribonucleoprotein (RNP) Delivery and “Cas RNA” Delivery

An engineered, non-naturally occurring system disclosed herein can be delivered into a cell by suitable methods known in the art, including but not limited to ribonucleoprotein (RNP) delivery and “Cas RNA” delivery described below.

In certain embodiments, a CRISPR-Cas system including a single guide nucleic acid and a Cas protein, or a CRISPR-Cas system including a targeter nucleic acid, a modulator nucleic acid, and a Cas protein, can be combined into a RNP complex and then delivered into the cell as a pre-formed complex. This method is suitable for active modification of the genetic or epigenetic information in a cell during a limited time period. For example, where the Cas protein has nuclease activity to modify the genomic DNA of the cell, the nuclease activity only needs to be retained for a period of time to allow DNA cleavage, and prolonged nuclease activity may increase off-targeting. Similarly, certain epigenetic modifications can be maintained in a cell once established and can be inherited by daughter cells.

A “ribonucleoprotein” or “RNP,” as used herein, can refer to a complex comprising a nucleoprotein and a ribonucleic acid. A “nucleoprotein” as provided herein can refer to a protein capable of binding a nucleic acid (e.g., RNA, DNA). Where the nucleoprotein binds a ribonucleic acid it can be referred to as “ribonucleoprotein.” The interaction between the ribonucleoprotein and the ribonucleic acid may be direct, e.g., by covalent bond, or indirect, e.g., by non-covalent bond (e.g. electrostatic interactions (e.g. ionic bond, hydrogen bond, halogen bond), van der Waals interactions (e.g. dipole-dipole, dipole-induced dipole, London dispersion), ring stacking (pi effects), hydrophobic interactions, or the like). In certain embodiments, the ribonucleoprotein includes an RNA-binding motif non-covalently bound to the ribonucleic acid. For example, positively charged aromatic amino acid residues (e.g., lysine residues) in the RNA-binding motif may form electrostatic interactions with the negative nucleic acid phosphate backbones of the RNA.

To ensure efficient loading of the Cas protein, the single guide nucleic acid, or the combination of the targeter nucleic acid and the modulator nucleic acid, can be provided in excess molar amount (e.g., at least 2 fold, at least 3 fold, at least 4 fold, or at least 5 fold) relative to the Cas protein. In certain embodiments, the targeter nucleic acid and the modulator nucleic acid are annealed under suitable conditions prior to complexing with the Cas protein. In other embodiments, the targeter nucleic acid, the modulator nucleic acid, and the Cas protein are directly mixed together to form an RNP.

A variety of delivery methods can be used to introduce an RNP disclosed herein into a cell. Exemplary delivery methods or vehicles include but are not limited to microinjection, liposomes (see, e.g., U.S. Pat. No. 10,829,787,) such as molecular trojan horses liposomes that delivers molecules across the blood brain barrier (see, Pardridge et al. (2010) Cold Spring Harb. Protoc., doi: 10.1101/pdb.prot5407), immunoliposomes, virosomes, microvesicles (e.g., exosomes and ARMMs), polycations, lipid: nucleic acid conjugates, electroporation, cell permeable peptides (see, U.S. Pat. No. 11,118,194), nanoparticles, nanowires (see, Shalek et al. (2012) Nano Letters, 12:6498), exosomes, and perturbation of cell membrane (e.g., by passing cells through a constriction in a microfluidic system, see, U.S. Pat. No. 11,125,739). Where the target cell is a proliferating cell, the efficiency of RNP delivery can be enhanced by cell cycle synchronization (see, U.S. Pat. No. 10,570,418). In certain embodiments, an RNP is delivered into a cell by electroporation.

In certain embodiments, a CRISPR-Cas system is delivered into a cell in a “approach, i.e., delivering (a) a single guide nucleic acid, or a combination of a targeter nucleic acid and a modulator nucleic acid, and (b) an RNA (e.g., messenger RNA (mRNA)) encoding a Cas protein. The RNA encoding the Cas protein can be translated in the cell and form a complex with the single guide nucleic acid or combination of the targeter nucleic acid and the modulator nucleic acid intracellularly. Similar to the RNP approach, RNAs have limited half-lives in cells, even though stability-increasing modification(s) can be made in one or more of the RNAs. Accordingly, the “Cas RNA” approach is suitable for active modification of the genetic or epigenetic information in a cell during a limited time period, such as DNA cleavage, and has the advantage of reducing off-targeting.

The mRNA can be produced by transcription of a DNA comprising a regulatory element operably linked to a Cas coding sequence. Given that multiple copies of Cas protein can be generated from one mRNA, the single guide nucleic acid, or the targeter nucleic acid and the modulator nucleic acid are generally provided in excess molar amount (e.g., at least 5 fold, at least 10 fold, at least 20 fold, at least 30 fold, at least 50 fold, or at least 100 fold) relative to the mRNA. In certain embodiments, the targeter nucleic acid and the modulator nucleic acid are annealed under suitable conditions prior to delivery into the cells. In other embodiments, the targeter nucleic acid and the modulator nucleic acid are delivered into the cells without annealing in vitro.

A variety of delivery systems can be used to introduce an “Cas RNA” system into a cell. Non-limiting examples of delivery methods or vehicles include microinjection, biolistic particles, liposomes (see, e.g., U.S. Pat. No. 10,829,787) such as molecular trojan horses liposomes that delivers molecules across the blood brain barrier (see, Pardridge et al. (2010) Cold Spring Harb. Protoc., doi:10.1101/pdb.prot5407), immunoliposomes, virosomes, polycations, lipid: nucleic acid conjugates, electroporation, nanoparticles, nanowires (see, Shalek et al. (2012) Nano Letters, 12:6498), exosomes, and perturbation of cell membrane (e.g., by passing cells through a constriction in a microfluidic system, see, U.S. Pat. No. 11,125,739). Specific examples of the “nucleic acid only” approach by electroporation are described in International (PCT) Publication No. WO 2016/164356.

In certain embodiments, the CRISPR-Cas system is delivered into a cell in the form of (a) a single guide nucleic acid or a combination of a targeter nucleic acid and a modulator nucleic acid, and (b) a DNA comprising a regulatory element operably linked to a Cas coding sequence. The DNA can be provided in a plasmid, viral vector, or any other form described in the “CRISPR Expression Systems” subsection. Such delivery method may result in constitutive expression of Cas protein in the target cell (e.g., if the DNA is maintained in the cell in an episomal vector or is integrated into the genome), and may increase the risk of off-targeting which is undesirable when the Cas protein has nuclease activity. Notwithstanding, this approach is useful when the Cas protein comprises a non-nuclease effector (e.g., a transcriptional activator or repressor). It is also useful for research purposes and for genome editing of plants.

### B. CRISPR Expression Systems

Also provided herein is a nucleic acid comprising a regulatory element operably linked to a nucleotide sequence encoding a guide nucleic acid disclosed herein. In certain embodiments, the nucleic acid comprises a regulatory element operably linked to a nucleotide sequence encoding a single guide nucleic acid: this nucleic acid alone can constitute a CRISPR expression system. In certain embodiments, the nucleic acid comprises a regulatory element operably linked to a nucleotide sequence encoding a targeter nucleic acid. In certain embodiments, the nucleic acid further comprises a nucleotide sequence encoding a modulator nucleic acid, wherein the nucleotide sequence encoding the modulator nucleic acid is operably linked to the same regulatory element as the nucleotide sequence encoding the targeter nucleic acid or a different regulatory element: this nucleic acid alone can constitute a CRISPR expression system.

In addition, the present invention provides a CRISPR expression system comprising: (a) a nucleic acid comprising a first regulatory element operably linked to a nucleotide sequence encoding a targeter nucleic acid and (b) a nucleic acid comprising a second regulatory element operably linked to a nucleotide sequence encoding a modulator nucleic acid.

In certain embodiments, a CRISPR expression system further comprises a nucleic acid comprising a third regulatory element operably linked to a nucleotide sequence encoding a Cas protein, such as a Cas protein disclosed herein. In certain embodiments, the Cas protein is a type V-A, type V-C, or type V-D Cas protein (e.g., Cas nuclease). In certain embodiments, the Cas protein is a type V-A Cas protein (e.g., Cas nuclease).

As used in this context, the term “operably linked” can mean that the nucleotide sequence of interest is linked to the regulatory element in a manner that allows for expression of the nucleotide sequence (e.g., in an in vitro transcription/translation system or in a host cell when the vector is introduced into the host cell).

The nucleic acids of a CRISPR expression system described above may be independently selected from various nucleic acids such as DNA (e.g., modified DNA) and RNA (e.g., modified RNA). In certain embodiments, the nucleic acids comprising a regulatory element operably linked to one or more nucleotide sequences encoding the guide nucleic acids are in the form of DNA. In certain embodiments, the nucleic acid comprising a third regulatory element operably linked to a nucleotide sequence encoding the Cas protein is in the form of DNA. The third regulatory element can be a constitutive or inducible promoter that drives the expression of the Cas protein. In other embodiments, the nucleic acid comprising a third regulatory element operably linked to a nucleotide sequence encoding the Cas protein is in the form of RNA (e.g., mRNA).

Nucleic acids of a CRISPR expression system can be provided in one or more vectors. The term “vector,” as used herein, can refer to a nucleic acid molecule capable of transporting another nucleic acid to which it has been linked. Conventional viral and non-viral based gene transfer methods can be used to introduce nucleic acids in cells, such as prokaryotic cells, eukaryotic cells, mammalian cells, or target tissues. Non-viral vector delivery systems include DNA plasmids, RNA (e.g. a transcript of a vector described herein), naked nucleic acid, and nucleic acid complexed with a delivery vehicle, such as a liposome. Viral vector delivery systems include DNA and RNA viruses, which have either episomal or integrated genomes after delivery to the cell. Gene therapy procedures are known in the art and disclosed in Van Brunt (1988) BIOTECHNOLOGY, 6:1149; Anderson (1992) SCIENCE, 256:808; Nabel & Feigner (1993) TIBTECH, 11:211; Mitani & Caskey (1993) TIBTECH, 11:162: Dillon (1993) TIBTECH, 11:167; Miller (1992) NATURE, 357:455: Vigne, (1995) RESTORATIVE NEUROLOGY AND NEUROSCIENCE, 8:35: Kremer & Perricaudet (1995) BRITISH MEDICAL BULLETIN, 51:31: Haddada et al. (1995) CURRENT TOPICS IN MICROBIOLOGY AND IMMUNOLOGY, 199:297: Yu et al. (1994) GENE THERAPY, 1:13; and Doerfler and Bohm (Eds.) (2012) The Molecular Repertoire of Adenoviruses II: Molecular Biology of Virus-Cell Interactions. In certain embodiments, at least one of the vectors is a DNA plasmid. In certain embodiments, at least one of the vectors is a viral vector (e.g., retrovirus, adenovirus, or adeno-associated virus).

Certain vectors are capable of autonomous replication in a host cell into which they are introduced (e.g., bacterial vectors having a bacterial origin of replication and episomal mammalian vectors). Other vectors (e.g., non-episomal mammalian vectors and replication defective viral vectors) do not autonomously replicate in the host cell. Certain vectors, however, may be integrated into the genome of the host cell and thereby are replicated along with the host genome. A skilled person in the art will appreciate that different vectors may be suitable for different delivery methods and have different host tropism, and will be able to select one or more vectors suitable for the use.

The term “regulatory element.” as used herein, can refer to a transcriptional and/or translational control sequence, such as a promoter, enhancer, transcription termination signal (e.g., polyadenylation signal), internal ribosomal entry sites (IRES), protein degradation signal, or the like, that provide for and/or regulate transcription of a non-coding sequence (e.g., a targeter nucleic acid or a modulator nucleic acid) or a coding sequence (e.g., a Cas protein) and/or regulate translation of an encoded polypeptide. Such regulatory elements are described, for example, in Goeddel, GENE EXPRESSION TECHNOLOGY: METHODS IN ENZYMOLOGY, 185, Academic Press, San Diego, Calif. (1990). Regulatory elements include those that direct constitutive expression of a nucleotide sequence in many types of host cell and those that direct expression of the nucleotide sequence only in certain host cells (e.g., tissue-specific regulatory sequences). A tissue-specific promoter may direct expression primarily in a desired tissue of interest, such as muscle, neuron, bone, skin, blood, specific organs (e.g., liver, pancreas), or particular cell types (e.g., lymphocytes). Regulatory elements may also direct expression in a temporal-dependent manner, such as in a cell-cycle dependent or developmental stage-dependent manner, which may or may not also be tissue or cell-type specific. In certain embodiments, a vector comprises one or more pol III promoter (e.g., 1, 2, 3, 4, 5, or more pol III promoters), one or more pol II promoters (e.g., 1, 2, 3, 4, 5, or more pol II promoters), one or more pol I promoters (e.g., 1, 2, 3, 4, 5, or more pol I promoters), or combinations thereof. Examples of pol III promoters include, but are not limited to, U6 and HI promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer), the SV40 promoter, the dihydrofolate reductase promoter, the B-actin promoter, the phosphoglycerol kinase (PGK) promoter, and the EF1α promoter. Also encompassed by the term “regulatory element” are enhancer elements, such as WPRE: CMV enhancers: the R-U5′ segment in LTR of HTLV-I (see, Takebe et al. (1988) MOL. CELL. BIOL., 8:466): SV40 enhancer; and the intron sequence between exons 2 and 3 of rabbit β-globin (see, O'Hare et al. (1981) PROC. NATL. ACAD. SCI. USA., 78:1527). It will be appreciated by those skilled in the art that the design of the expression vector can depend on factors such as the choice of the host cell to be transformed, the level of expression desired, etc. A vector can be introduced into host cells to produce transcripts, proteins, or peptides, including fusion proteins or peptides, encoded by nucleic acids as described herein (e.g., CRISPR transcripts, proteins, enzymes, mutant forms thereof, or fusion proteins thereof).

In certain embodiments, the nucleotide sequence encoding the Cas protein is codon optimized for expression in a prokaryotic cell, e.g., E coli, eukaryotic host cell, e.g., a yeast cell (e.g., S, cerevisiae), a mammalian cell (e.g., a mouse cell, a rat cell, or a human cell), or a plant cell. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at kazusa.or.jp/codon/and these tables can be adapted in a number of ways (see, Nakamura et al. (2000) NUCL. ACIDS RES., 28:292). Computer algorithms for codon optimizing a particular sequence for expression in a particular host cell, such as Gene Forge (Aptagen: Jacobus, Pa.), are also available. In certain embodiments, the codon optimization facilitates or improves expression of the Cas protein in the host cell.

### C. Donor Templates

Cleavage of a target nucleotide sequence in the genome of a cell by a CRISPR-Cas system or complex can activate DNA damage pathways, which may rejoin the cleaved DNA fragments by NHEJ or HDR. HDR requires a repair template, either endogenous or exogenous, to transfer the sequence information from the repair template to the target.

In certain embodiments, an engineered, non-naturally occurring system or CRISPR expression system further comprises a donor template. As used herein, the term “donor template” can refer to a nucleic acid designed to serve as a repair template at or near the target nucleotide sequence upon introduction into a cell or organism. In certain embodiments, the donor template is complementary to a polynucleotide comprising the target nucleotide sequence or a portion thereof. When optimally aligned, a donor template may overlap with one or more nucleotides of a target nucleotide sequences (e.g. about or more than about 1, 5, 10, 15, 20, 25, 30, 35, 40, or more nucleotides). The nucleotide sequence of the donor template is typically not identical to the genomic sequence that it replaces. Rather, the donor template may contain one or more substitutions, insertions, deletions, inversions or rearrangements with respect to the genomic sequence, so long as sufficient homology is present to support homology-directed repair. In certain embodiments, the donor template comprises a non-homologous sequence flanked by two regions of homology (i.e., homology arms), such that homology-directed repair between the target DNA region and the two flanking sequences results in insertion of the non-homologous sequence at the target region. In certain embodiments, the donor template comprises a non-homologous sequence 10-100 nucleotides, 50-500 nucleotides, 100-1,000 nucleotides, 200-2,000 nucleotides, or 500-5,000 nucleotides in length positioned between two homology arms.

Generally, the homologous region(s) of a donor template has at least 50% sequence identity to a genomic sequence with which recombination is desired. The homology arms are designed or selected such that they are capable of recombining with the nucleotide sequences flanking the target nucleotide sequence under intracellular conditions. In certain embodiments, where HDR of the non-target strand is desired, the donor template comprises a first homology arm homologous to a sequence 5′ to the target nucleotide sequence and a second homology arm homologous to a sequence 3′ to the target nucleotide sequence. In certain embodiments, the first homology arm is at least 50% (e.g., at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%) identical to a sequence 5′ to the target nucleotide sequence. In certain embodiments, the second homology arm is at least 50% (e.g., at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%) identical to a sequence 3′ to the target nucleotide sequence. In certain embodiments, when the donor template sequence and a polynucleotide comprising a target nucleotide sequence are optimally aligned, the nearest nucleotide of the donor template is within about 1, 5, 10, 15, 20, 25, 50, 75, 100, 200, 300, 400, 500, 1000, 2000, 3000, 4000, or more nucleotides from the target nucleotide sequence.

In certain embodiments, the donor template further comprises an engineered sequence not homologous to the sequence to be repaired. Such engineered sequence can harbor a barcode and/or a sequence capable of hybridizing with a donor template-recruiting sequence disclosed herein.

In certain embodiments, the donor template further comprises one or more mutations relative to the genomic sequence, wherein the one or more mutations reduce or prevent cleavage, by the same CRISPR-Cas system, of the donor template or of a modified genomic sequence with at least a portion of the donor template sequence incorporated. In certain embodiments, in the donor template, the PAM adjacent to the target nucleotide sequence and recognized by the Cas nuclease is mutated to a sequence not recognized by the same Cas nuclease. In certain embodiments, in the donor template, the target nucleotide sequence (e.g., the seed region) is mutated. In certain embodiments, the one or more mutations are silent with respect to the reading frame of a protein-coding sequence encompassing the mutated sites.

The donor template can be provided to the cell as single-stranded DNA, single-stranded RNA, double-stranded DNA, or double-stranded RNA. It is understood that a CRISPR-Cas system, such as a system disclosed herein, may possess nuclease activity to cleave the target strand, the non-target strand, or both. When HDR of the target strand is desired, a donor template having a nucleic acid sequence complementary to the target strand is also contemplated.

The donor template can be introduced into a cell in linear or circular form. If introduced in linear form, the ends of the donor template may be protected (e.g., from exonucleolytic degradation) by methods known to those of skill in the art. For example, one or more dideoxynucleotide residues are added to the 3′ terminus of a linear molecule and/or self-complementary oligonucleotides are ligated to one or both ends (see, for example, Chang et al. (1987) PROC. NATL. ACAD SCI USA, 84:4959; Nehls et al. (1996) SCIENCE, 272:886; see also the chemical modifications for increasing stability and/or specificity of RNA disclosed supra). Additional methods for protecting exogenous polynucleotides from degradation include, but are not limited to, addition of terminal amino group(s) and the use of modified internucleotide linkages such as, for example, phosphorothioates, phosphoramidates, and O-methyl ribose or deoxyribose residues. As an alternative to protecting the termini of a linear donor template, additional lengths of sequence may be included outside of the regions of homology that can be degraded without impacting recombination.

A donor template can be a component of a vector as described herein, contained in a separate vector, or provided as a separate polynucleotide, such as an oligonucleotide, linear polynucleotide, or synthetic polynucleotide. In certain embodiments, the donor template is a DNA. In certain embodiments, a donor template is in the same nucleic acid as a sequence encoding the single guide nucleic acid, a sequence encoding the targeter nucleic acid, a sequence encoding the modulator nucleic acid, and/or a sequence encoding the Cas protein, where applicable. In certain embodiments, a donor template is provided in a separate nucleic acid. A donor template polynucleotide may be of any suitable length, such as about or at least about 50, 75, 100, 150, 200, 500, 1000, 2000, 3000, 4000, or more nucleotides in length.

A donor template can be introduced into a cell as an isolated nucleic acid. Alternatively, a donor template can be introduced into a cell as part of a vector (e.g., a plasmid) having additional sequences such as, for example, replication origins, promoters and genes encoding antibiotic resistance, that are not intended for insertion into the DNA region of interest. Alternatively, a donor template can be delivered by viruses (e.g., adenovirus, adeno-associated virus (AAV)). In certain embodiments, the donor template is introduced as an AAV, e.g., a pseudotyped AAV. The capsid proteins of the AAV can be selected by a person skilled in the art based upon the tropism of the AAV and the target cell type. For example, in certain embodiments, the donor template is introduced into a hepatocyte as AAV8 or AAV9. In certain embodiments, the donor template is introduced into a hematopoietic stem cell, a hematopoietic progenitor cell, or a T lymphocyte (e.g., CD8+ T lymphocyte) as AAV6 or an AAVHSC (see, U.S. Pat. No. 9,890,396). It is understood that the sequence of a capsid protein (VP1, VP2, or VP3) may be modified from a wild-type AAV capsid protein, for example, having at least 50% (e.g., at least 60%, at least 70%, at least 75%, at least 80%, at least 85%, at least 90%, at least 91%, at least 92%, at least 93%, at least 94%, at least 95%, at least 96%, at least 97%, at least 98%, or at least 99%) sequence identity to a wild-type AAV capsid sequence.

The donor template can be delivered to a cell (e.g., a primary cell) by various delivery methods, such as a viral or non-viral method disclosed herein. In certain embodiments, a non-viral donor template is introduced into the target cell as a naked nucleic acid or in complex with a liposome or poloxamer. In certain embodiments, a non-viral donor template is introduced into the target cell by electroporation. In other embodiments, a viral donor template is introduced into the target cell by infection. The engineered, non-naturally occurring system can be delivered before, after, or simultaneously with the donor template (see, International (PCT) Application Publication No. WO 2017/053729). A skilled person in the art will be able to choose proper timing based upon the form of delivery (consider, for example, the time needed for transcription and translation of RNA and protein components) and the half-life of the molecule(s) in the cell. In particular embodiments, where the CRISPR-Cas system including the Cas protein is delivered by electroporation (e.g., as an RNP), the donor template (e.g., as an AAV) is introduced into the cell within 4 hours (e.g., within 1, 2, 3, 4, 5, 6, 7, 8, 9, 10, 11, 12, 13, 14, 15, 16, 17, 18, 19, 20, 25, 30, 35, 40, 45, 50, 55, 60, 90, 120, 150, 180, 210, or 240 minutes) after the introduction of the engineered, non-naturally occurring system.

In certain embodiments, the donor template is conjugated covalently to a modulator nucleic acid. Covalent linkages suitable for this conjugation are known in the art and are described, for example, in U.S. Pat. No. 9,982,278 and Savic et al. (2018) ELIFE 7: e33761. In certain embodiments, the donor template is covalently linked to a modulator nucleic acid (e.g., the 5′ end of the modulator nucleic acid) through an internucleotide bond. In certain embodiments, the donor template is covalently linked to a modulator nucleic acid (e.g., the 5′ end of the modulator nucleic acid) through a linker.

In certain embodiments, the donor template can comprise any nucleic acid chemistry. In certain embodiments, the donor template can comprise DNA and/or RNA nucleotides. In certain embodiments, the donor template can comprise single-stranded DNA, linear single-stranded RNA, linear double-stranded DNA, linear double-stranded RNA, circular single-stranded DNA, circular single-stranded RNA, circular double-stranded DNA, or circular double-stranded RNA. In certain embodiments, the donor template comprises a mutation in a PAM sequence to partially or completely abolish binding of the RNP to the DNA. In certain embodiments, the donor template is present at a concentration of at least 0.05, 0.01, 0.2, 0.3, 0.4, 0.5, 0.6, 0.7, 0.8, 0.9, 1, 1.25, 1.5, 1.75, 2, 3, or 4, and/or no more than 0.01, 0.2, 0.3, 0.4, 0.5, 0.6, 0.7, 0.8, 0.9, 1, 1.25, 1.5, 1.75, 2, 3, 4, or 5 μg μL⁻¹, for example 0.01-5 μg μL⁻¹. In certain embodiments, the donor template comprises one or more promoters. In certain embodiments, the donor template comprises a promoter that shares at least 70%, at least 75%, at least 80%, at least 85%, at least 90%, at least 95%, at least 99.5% sequence identity with any one of SEQ ID NOs: 78-85 of Table 4.

TABLE 4
Promoter sequences

| | SEQ ID | |
|---|---|---|
| Name | NO | Sequence |
| CMV | 78 | CGTTACATAACTTACGGTAAATGGCCCGCCTGGCTGACCGCCCAACG |
| | | ACCCCCGCCCATTGACGTCAATAATGACGTATGTTCCCATAGTAACG |
| | | CCAATAGGGACTTTCCATTGACGTCAATGGGTGGAGTATTTACGGTA |
| | | AACTGCCCACTTGGCAGTACATCAAGTGTATCATATGCCAAGTACGC |
| | | CCCCTATTGACGTCAATGACGGTAAATGGCCCGCCTGGCATTATGCC |
| | | CAGTACATGACCTTATGGGACTTTCCTACTTGGCAGTACATCTACGT |
| | | ATTAGTCATCGCTATTACCATGGTGATGCGGTTTTGGCAGTACATCA |
| | | ATGGGCGTGGATAGCGGTTTGACTCACGGGGATTTCCAAGTCTCCAC |
| | | CCCATTGACGTCAATGGGAGTTTGTTTTGGCACCAAAATCAACGGGA |
| | | CTTTCCAAAATGTCGTAACAACTCCGCCCCATTGACGCAAATGGGCG |
| | | GTAGGCGTGTACGGTGGGAGGTCTATATAAGCAGAGCT |
| SCP | 79 | GTACTTATATAAGGGGGTGGGGGCGCGTTCGTCCTCAGTCGCGATCG |
| | | AACACTCGAGCCGAGCAGACGTGCCTACGGACCG |
| CMVe- | 80 | CGTTACATAACTTACGGTAAATGGCCCGCCTGGCTGACCGCCCAACG |
| SCP | | ACCCCCGCCCATTGACGTCAATAATGACGTATGTTCCCATAGTAACG |
| | | CCAATAGGGACTTTCCATTGACGTCAATGGGTGGAGTATTTACGGTA |
| | | AACTGCCCACTTGGCAGTACATCAAGTGTATCATATGCCAAGTACGC |
| | | CCCCTATTGACGTCAATGACGGTAAATGGCCCGCCTGGCATTATGCC |
| | | CAGTACATGACCTTATGGGACTTTCCTACTTGGCAGTACATCTACGT |
| | | ATTAGTCATCGCTATTACCATGGTACTTATATAAGGGGGTGGGGGCG |
| | | CGTTCGTCCTCAGTCGCGATCGAACACTCGAGCCGAGCAGACGTGCC |
| | | TACGGACCG |
| CMVmax | 81 | TCAATATTGGCCATTAGCCATATTATTCATTGGTTATATAGCATAAA |
| | | TCAATATTGGCTATTGGCCATTGCATACGTTGTATCTATATCATAAT |
| | | ATGTACATTTATATTGGCTCATGTCCAATATGACCGCCATGTTGGCA |
| | | TTGATTATTGACTAGTTATTAATAGTAATCAATTACGGGGTCATTAG |
| | | TTCATAGCCCATATATGGAGTTCCGCGTTACATAACTTACGGTAAAT |
| | | GGCCCGCCTGGCTGACCGCCCAACGACCCCCGCCCATTGACGTCAAT |
| | | AATGACGTATGTTCCCATAGTAACGCCAATAGGGACTTTCCATTGAC |
| | | GTCAATGGGTGGAGTATTTACGGTAAACTGCCCACTTGGCAGTACAT |
| | | CAAGTGTATCATATGCCAAGTCCGCCCCCTATTGACGTCAATGACGG |
| | | TAAATGGCCCGCCTGGCATTATGCCCAGTACATGACCTTACGGGACT |
| | | TTCCTACTTGGCAGTACATCTACGTATTAGTCATCGCTATTACCATG |
| | | GTGATGCGGTTTTGGCAGTACACCAATGGGCGTGGATAGCGGTTTGA |
| | | CTCACGGGGATTTCCAAGTCTCCACCCCATTGACGTCAATGGGAGTT |
| | | TGTTTTGGCACCAAAATCAACGGGACTTTCCAAAATGTCGTAATAAC |
| | | CCCGCCCCGTTGACGCAAATGGGCGGTAGGCGTGTACGGTGGGAGGT |
| | | CTATATAAGCAGAGGTCGTTTAGTGAACCGTCAGATCACTAGTAGCT |
| | | TTATTGCGGTAGTTTATCACAGTTAAATTGCTAACGCAGTCAGTGCT |
| | | CGACTGATCACAGGTAAGTATCAAGGTTACAAGACAGGTTTAAGGAG |
| | | GCCAATAGAAACTGGGCTTGTCGAGACAGAGAAGATTCTTGCGTTTC |
| | | TGATAGGCACCTATTGGTCTTACTGACATCCACTTTGCCTTTCTCTC |
| | | CACAGGG |
| JET | 82 | GAATTCGGGCGGAGTTAGGGCGGAGCCAATCAGCGTGCGCCGTTCCG |
| | | AAAGTTGCCTTTTATGGCTGGGCGGAGAATGGGCGGTGAACGCCGAT |
| | | GATTATATAAGGACGCGCCGGGTGTGGCACAGCTAGTTCCGTCGCAG |
| | | CCGGGATTTGGGTCGCGGTTCTTGTTTGTGGATCCCTGTGATCGTCA |
| | | CTTGACA |
| CAG | 83 | ATCTCGACTAGTTATTAATAGTAATCAATTACGGGGTCATTAGTTCA |
| | | TAGCCCATATATGGAGTTCCGCGTTACATAACTTACGGTAAATGGCC |
| | | CGCCTGGCTGACCGCCCAACGACCCCCGCCCATTGACGTCAATAATG |
| | | ACGTATGTTCCCATAGTAACGCCAATAGGGACTTTCCATTGACGTCA |
| | | ATGGGTGGAGTATTTACGGTAAACTGCCCACTTGGCAGTACATCAAG |
| | | TGTATCATATGCCAAGTACGCCCCCTATTGACGTCAATGACGGTAAA |
| | | TGGCCCGCCTGGCATTATGCCCAGTACATGACCTTATGGGACTTTCC |
| | | TACTTGGCAGTACATCTACGTATTAGTCATCGCTATTACCATGGTCG |
| | | AGGTGAGCCCCACGTTCTGCTTCACTCTCCCCATCTCCCCCCCCTCC |
| | | CCACCCCCAATTTTGTATTTATTTATTTTTTAATTATTTTGTGCAGC |
| | | GATGGGGGCGGGGGGGGGGGGGGGGCGCGCGCCAGGCGGGGCGGGGC |
| | | GGGGCGAGGGGCGGGGCGGGGCGAGGCGGAGAGGTGCGGCGGCAGCC |
| | | AATCAGAGCGGCGCGCTCCGAAAGTTTCCTTTTATGGCGAGGCGGCG |
| | | GCGGCGGCGGCCCTATAAAAAGCGAAGCGCGCGGCGGGGGGGGAGTC |
| | | GCTGCGACGCTGCCTTCGCCCCGTGCCCCGCTCCGCCGCCGCCTCGC |
| | | GCCGCCCGCCCCGGCTCTGACTGACCGCGTTACTCCCACAGGTGAGC |
| | | GGGCGGGACGGCCCTTCTCCTCCGGGCTGTAATTAGCGCTTGGTTTA |
| | | ATGACGGCTTGTTTCTTTTCTGTGGCTGCGTGAAAGCCTTGAGGGGC |
| | | TCCGGGAGGGCCCTTTGTGCGGGGGGAGCGGCTCGGGGGGTGCGTGC |
| | | GTGTGTGTGTGCGTGGGGAGCGCCGCGTGCGGCTCCGCGCTGCCCGG |
| | | CGGCTGTGAGCGCTGCGGGCGCGGCGCGGGGCTTTGTGCGCTCCGCA |
| | | GTGTGCGCGAGGGGAGCGCGGCCGGGGGCGGTGCCCCGCGGTGCGGG |
| | | GGGGGCTGCGAGGGGAACAAAGGCTGCGTGCGGGGTGTGTGCGTGGG |
| | | GGGGTGAGCAGGGGGTGTGGGCGCGTCGGTCGGGCTGCAACCCCCCC |
| | | TGCACCCCCCTCCCCGAGTTGCTGAGCACGGCCCGGCTTCGGGTGCG |
| | | GGGCTCCGTACGGGGCGTGGCGCGGGGCTCGCCGTGCCGGGCGGGGG |
| | | GTGGCGGCAGGTGGGGGTGCCGGGGGGGGCGGGGCCGCCTCGGGCCG |
| | | GGGAGGGCTCGGGGGAGGGGCGCGGCGGCCCCCGGAGCGCCGGCGGC |
| | | TGTCGAGGCGCGGCGAGCCGCAGCCATTGCCTTTTATGGTAATCGTG |
| | | CGAGAGGGCGCAGGGACTTCCTTTGTCCCAAATCTGTGCGGAGCCGA |
| | | AATCTGGGAGGCGCCGCCGCACCCCCTCTAGCGGGCGCGGGGCGAAG |
| | | CGGTGCGGCGCCGGCAGGAAGGAAATGGGGGGGGAGGGCCTTCGTGC |
| | | GTCGCCGCGCCGCCGTCCCCTTCTCCCTCTCCAGCCTCGGGGCTGTC |
| | | CGCGGGGGGACGGCTGCCTTCGGGGGGGACGGGGCAGGGGGGGGTTC |
| | | GGCTTCTGGCGTGTGACCGGCGGCTCTAGAGCCTCTGCTAACCATGT |
| | | TCATGCCTTCTTCTTTTTCCTACAGCTCCTGGGCAACGTGCTGGTTA |
| | | TTGTGCTGTCTCATCATTTTGGCAAAGAATT |
| PGK | 84 | GGGGTTGGGGTTGCGCCTTTTCCAAGGCAGCCCTGGGTTTGCGCAGG |
| | | GACGCGGCTGCTCTGGGCGTGGTTCCGGGAAACGCAGCGGCGCCGAC |
| | | CCTGGGTCTCGCACATTCTTCACGTCCGTTCGCAGCGTCACCCGGAT |
| | | CTTCGCCGCTACCCTTGTGGGCCCCCCGGCGACGCTTCCTGCTCCGC |
| | | CCCTAAGTCGGGAAGGTTCCTTGCGGTTCGCGGCGTGCCGGACGTGA |
| | | CAAACGGAAGCCGCACGTCTCACTAGTACCCTCGCAGACGGACAGCG |
| | | CCAGGGAGCAATGGCAGCGCGCCGACCGCGATGGGCTGTGGCCAATA |
| | | GCGGCTGCTCAGCAGGGCGCGCCGAGAGCAGCGGCCGGGAAGGGGCG |
| | | GTGCGGGAGGCGGGGTGTGGGGCGGTAGTGTGGGCCCTGTTCCTGCC |
| | | CGCGCGGTGTTCCGCATTCTGCAAGCCTCCGGAGCGCACGTCGGCAG |
| | | TCGGCTCCCTCGTTGACCGAATCACCGACCTCTCTCCCCAG |
| EF-1a | 85 | GAATTCAGGCTCCGGTGCCCGTCAGTGGGCAGAGCGCACATCGCCCA |
| | | CAGTCCCCGAGAAGTTGGGGGGAGGGGTCGGCAATTGAACCGGTGCC |
| | | TAGAGAAGGTGGCGCGGGGTAAACTGGGAAAGTGATGTCGTGTACTG |
| | | GCTCCGCCTTTTTCCCGAGGGTGGGGGAGAACCGTATATAAGTGCAG |
| | | TAGTCGCCGTGAACGTTCTTTTTCGCAACGGGTTTGCCGCCAGAACA |
| | | CAGGTAAGTGCCGTGTGTGGTTCCCGCGGGCCTGGCCTCTTTACGGG |
| | | TTATGGCCCTTGCGTGCCTTGAATTACTTCCACCTGGCTGCAGTACG |
| | | TGATTCTTGATCCCGAGCTTCGGGTTGGAAGTGGGTGGGAGAGTTCG |
| | | AGGCCTTGCGCTTAAGGAGCCCCTTCGCCTCGTGCTTGAGTTGAGGC |
| | | CTGGCCTGGGCGCTGGGGCCGCCGCGTGCGAATCTGGTGGCACCTTC |
| | | GCGCCTGTCTCGCTGCTTTCGATAAGTCTCTAGCCATTTAAAATTTT |
| | | TGATGACCTGCTGCGACGCTTTTTTTCTGGCAAGATAGTCTTGTAAA |
| | | TGCGGGCCAAGATCTGCACACTGGTATTTCGGTTTTTGGGGCCGCGG |
| | | GCGGCGACGGGGCCCGTGCGTCCCAGCGCACATGTTCGGCGAGGCGG |
| | | GGCCTGCGAGCGCGGCCACCGAGAATCGGACGGGGGTAGTCTCAAGC |
| | | TGGCCGGCCTGCTCTGGTGCCTGGTCTCGCGCCGCCGTGTATCGCCC |
| | | CGCCCTGGGCGGCAAGGCTGGCCCGGTCGGCACCAGTTGCGTGAGCG |
| | | GAAAGATGGCCGCTTCCCGGCCCTGCTGCAGGGAGCTCAAAATGGAG |
| | | GACGCGGCGCTCGGGAGAGCGGGCGGGTGAGTCACCCACACAAAGGA |
| | | AAAGGGCCTTTCCGTCCTCAGCCGTCGCTTCATGTGACTCCACGGAG |
| | | TACCGGGCGCCGTCCAGGCACCTCGATTAGTTCTCGAGCTTTTGGAG |
| | | TACGTCGTCTTTAGGTTGGGGGGAGGGGTTTTATGCGATGGAGTTTC |
| | | CCCACACTGAGTGGGTGGAGACTGAAGTTAGGCCAGCTTGGCACTTG |
| | | ATGTAATTCTCCTTGGAATTTGCCCTTTTTGAGTTTGGATCTTGGTT |
| | | CATTCTCAAGCCTCAGACAGTGGTTCAAAGTTTTTTTCTTCCATTTC |
| | | AGGTGTCGTGACATCATTTT |

D. Efficiency and specificity

An engineered, non-naturally occurring system can be evaluated in terms of efficiency and/or specificity in nucleic acid targeting, cleavage, or modification.

In certain embodiments, an engineered, non-naturally occurring system has high efficiency. For example, in certain embodiments, at least 1%, at least 1.5%, at least 2%, at least 2.5%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, or at least 99% of a population of nucleic acids having the target nucleotide sequence and a cognate PAM, when contacted with the engineered, non-naturally occurring system, is targeted, cleaved, or modified. In certain embodiments, the genomes of at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, or at least 99% of a population of cells, when the engineered, non-naturally occurring system is delivered into the cells, are targeted, cleaved, or modified.

It has been observed that for a given spacer sequence, the occurrence of on-target events and the occurrence of off-target events are generally correlated. For certain therapeutic purposes, lower on-target efficiency can be tolerated and low off-target frequency is more desirable. For example, when editing or modifying a proliferating cell that will be delivered to a subject and proliferate in vivo, tolerance to off-target events is low. Prior to delivery, it is possible to assess the on-target and off-target events, thereby selecting one or more colonies that have the desired edit or modification and lack any undesired edit or modification. Notwithstanding, the on-target efficiency may need to meet a certain standard to be suitable for therapeutic use. High editing efficiency in a standard CRISPR-Cas system allows tuning of the system, for example, by reducing the binding of the guide nucleic acids to the Cas protein, without losing therapeutic applicability.

In certain embodiments, when a population of nucleic acids having the target nucleotide sequence and a cognate PAM is contacted with the engineered, non-naturally occurring system disclosed herein, the frequency of off-target events (e.g., targeting, cleavage, or modification, depending on the function of the CRISPR-Cas system) is reduced. Methods of assessing off-target events were summarized in Lazzarotto et al. (2018) Nat Protoc. 13 (11): 2615-42, and include discovery of in situ Cas off-targets and verification by sequencing (DISCOVER-seq) as disclosed in Wienert et al. (2019) Science 364 (6437): 286-89: genome-wide unbiased identification of double-stranded breaks (DSBs) enabled by sequencing (GUIDE-seq) as disclosed in Kleinstiver et al. (2016) Nat. Biotech. 34:869-74; circularization for in vitro reporting of cleavage effects by sequencing (CIRCLE-seq) as described in Kocak et al. (2019) Nat. Biotech. 37:657-66. In certain embodiments, the off-target events include targeting, cleavage, or modification at a given off-target locus (e.g., the locus with the highest occurrence of off-target events detected). In certain embodiments, the off-target events include targeting, cleavage, or modification at all the loci with detectable off-target events, collectively.

In certain embodiments, genomic mutations are detected in no more than 0.0001%, 0.0002%, 0.0003%, 0.0004%, 0.0005%, 0.0006%, 0.0007%, 0.0008%, 0.0009%, 0.001%, 0.002%, 0.003%, 0.004%, 0.005%, 0.006%, 0.007%, 0.008%, 0.009%, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, or 5% of the cells at any off-target loci (in aggregate). In certain embodiments, the ratio of the percentage of cells having an on-target event to the percentage of cells having any off-target event (e.g., the ratio of the percentage of cells having an on-target editing event to the percentage of cells having a mutation at any off-target loci) is at least 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, or 10000. It is understood that genetic variation may be present in a population of cells, for example, by spontaneous mutations, and such mutations are not included as off-target events.

### Multiplexing

The method of targeting, editing, and/or modifying a genomic DNA disclosed herein can be conducted in multiplicity. For example, a library of targeter nucleic acids can be used to target multiple genomic loci: a library of donor templates can also be used to generate multiple insertions, deletions, and/or substitutions. The multiplex assay can be conducted in a screening method wherein each separate cell culture (e.g., in a well of a 96-well plate or a 384-well plate) is exposed to a different guide nucleic acid having a different targeter stem sequence and/or a different donor template. The multiplex assay can also be conducted in a selection method wherein a cell culture is exposed to a mixed population of different guide nucleic acids and/or donor templates, and the cells with desired characteristics (e.g., functionality) are enriched or selected by advantageous survival or growth, resistance to a certain agent, expression of a detectable protein (e.g., a fluorescent protein that is detectable by flow cytometry), etc.

In certain embodiments, the plurality of guide nucleic acids and/or the plurality of donor templates are designed for saturation editing. For example, in certain embodiments, each nucleotide position in a sequence of interest is systematically modified with each of all four traditional bases, A, T, G and C. In other embodiments, at least one sequence in each gene from a pool of genes of interest is modified, for example, according to a CRISPR design algorithm. In certain embodiments, each sequence from a pool of exogenous elements of interest (e.g., protein coding sequences, non-protein coding genes, regulatory elements) is inserted into one or more given loci of the genome.

It is understood that the multiplex methods suitable for the purpose of carrying out a screening or selection method, which is typically conducted for research purposes, may be different from the methods suitable for therapeutic purposes. For example, constitutive expression of certain elements (e.g., a Cas nuclease and/or a guide nucleic acid) may be undesirable for therapeutic purposes due to the potential of increased off-targeting. Conversely, for research purposes, constitutive expression of a Cas nuclease and/or a guide nucleic acid may be desirable. For example, the constitutive expression provides a large window during which other elements can be introduced. When a stable cell line is established for the constitutive expression, the number of exogenous elements that need to be co-delivered into a single cell is also reduced. Therefore, constitutive expression of certain elements can increase the efficiency and reduce the complexity of a screening or selection process. Inducible expression of certain elements of the system disclosed herein may also be used for research purposes given similar advantages. Expression may be induced by an exogenous agent (e.g., a small molecule) or by an endogenous molecule or complex present in a particular cell type (e.g., at a particular stage of differentiation). Methods known in the art, such as those described herein, can be used for constitutively or inducibly expressing one or more elements. For example, the specificity of CRISPR nucleases is at least partially dictated by the uniqueness of the spacer (in combination with spacer sequence's proximity to a requisite PAM) and its off-target score can be calculated with algorithms, such as crispr.mit.edu (Hsu et al. (2013) Nat. Biotech. 31:827-832). The highest possible score is 100, which shows probability for high specificity and few off targets. Because our SHS library targets intergenic regions, the algorithm for gRNA prediction should be able to make alignments with repeated regions and low-complexity sequences.

It is further understood that despite the need to introduce multiple elements—the single guide nucleic acid and the Cas protein: or the targeter nucleic acid, the modulator nucleic acid, and the Cas protein—these elements can be delivered into the cell as a single complex of pre-formed RNP. Therefore, the efficiency of the screening or selection process can also be achieved by pre-assembling a plurality of RNP complexes in a multiplex manner.

In certain embodiments, the method disclosed herein further comprises a step of identifying a guide nucleic acid, a Cas protein, a donor template, or a combination of two or more of these elements from the screening or selection process. A set of barcodes may be used, for example, in the donor template between two homology arms, to facilitate the identification. In specific embodiments, the method further comprises harvesting the population of cells; selectively amplifying a genomic DNA or RNA sample including the target nucleotide sequence(s) and/or the barcodes; and/or sequencing the genomic DNA or RNA sample and/or the barcodes that has been selectively amplified.

In addition, the present invention provides a library comprising a plurality of guide nucleic acids, such as a plurality of guide nucleic acids disclosed herein. In another aspect, the present invention provides a library comprising a plurality of nucleic acids each comprising a regulatory element operably linked to a different guide nucleic acid such as a different guide nucleic acid disclosed herein. These libraries can be used in combination with one or more Cas proteins or Cas-coding nucleic acids, such as disclosed herein, and/or one or more donor templates, such as disclosed herein, for a screening or selection method.

### E. Genes to be Modified

The gene to be targeted in a genome can be any suitable gene. A spacer sequence for use in a gRNA system that also includes one or more of the ssODN compositions provided herein can thus be capable of hybridizing with any suitable gene. Non-limiting examples of genes include human ADORA2A, ALPNR, B2M, BBS1, CALR, CARD11, CD3G, CD52, CD58, CD247, CIITA, COL17A1, CSF1R, CTLA4, DCK, DEFB134, DHODH, ERAP1, ERAP2, FAS, mir-101-2, HAVCR2 (also called TIM3), IFNGR1, IFNGR2, IL7R, JAK1, JAK2, LAG3, LCK, LCK1, MLANA, MVD, PDCD1 (also called PD-1), PLCG1, PLK1, PSMB5, PSMB8, PSMB9, PTCD2, PTPNI, PTPN6, PTPN11, RFX5, RFXAP, RPL23, RXANK, SOX10, SRP54, STAT1, Tap1, TAP2, TAPBP, TGFBR2, TIGIT, TIM3, TRAC, TRBC1, TRBC1+2, TRBC2, TUBB, TWF1, U6. Further non-limiting examples include CSF2, CD40LG, CD3E, and CD38.

In certain embodiments of the engineered, non-naturally occurring system, the spacer sequence comprises a nucleotide sequence selected from the group consisting of SEQ ID NOs: 983, 1024-1030, and 1084-1105, wherein the spacer sequence is capable of hybridizing with the human ADORA2A gene. In certain embodiments, when the system is delivered into a population of human cells ex vivo, the genomic sequence at the ADORA2A gene locus is edited in at least 1.5% of the cells.

In certain embodiments of the engineered, non-naturally occurring system, the spacer sequence comprises a nucleotide sequence selected from the group consisting of SEQ ID NOs: 1549-1558, wherein the spacer sequence is capable of hybridizing with the human APLNR gene. In certain embodiments, when the system is delivered into a population of human cells ex vivo, the genomic sequence at the APLNR gene locus is edited in at least 1.5% of the cells.

In certain embodiments of the engineered, non-naturally occurring system, the spacer sequence comprises a nucleotide sequence selected from the group consisting of SEQ ID NOs: 984, 989-991, 1031-1038, 1106-1115, and 1285-1302, wherein the spacer sequence is capable of hybridizing with the human B2M gene. In certain embodiments, when the system is delivered into a population of human cells ex vivo, the genomic sequence at the B2M gene locus is edited in at least 1.5% of the cells.

In certain embodiments of the engineered, non-naturally occurring system, the spacer sequence comprises a nucleotide sequence selected from the group consisting of SEQ ID NOs: 1559-1568, wherein the spacer sequence is capable of hybridizing with the human BBSI gene. In certain embodiments, when the system is delivered into a population of human cells ex vivo, the genomic sequence at the BBS1 gene locus is edited in at least 1.5% of the cells.

In certain embodiments of the engineered, non-naturally occurring system, the spacer sequence comprises a nucleotide sequence selected from the group consisting of SEQ ID NOs: 1539-1548, wherein the spacer sequence is capable of hybridizing with the human CALR gene. In certain embodiments, when the system is delivered into a population of human cells ex vivo, the genomic sequence at the CALR gene locus is edited in at least 1.5% of the cells.

In certain embodiments of the engineered, non-naturally occurring system, the spacer sequence comprises a nucleotide sequence selected from the group consisting of SEQ ID NOs: 1388-1390, wherein the spacer sequence is capable of hybridizing with the human CARD11 gene. In certain embodiments, when the system is delivered into a population of human cells ex vivo, the genomic sequence at the CARD11 gene locus is edited in at least 1.5% of the cells.

In certain embodiments of the engineered, non-naturally occurring system, the spacer sequence comprises a nucleotide sequence selected from the group consisting of SEQ ID NOs: 86-111 and 1528, wherein the spacer sequence is capable of hybridizing with the human CD247 gene. In certain embodiments, when the system is delivered into a population of human cells ex vivo, the genomic sequence at the CD247 gene locus is edited in at least 1.5% of the cells.

In certain embodiments of the engineered, non-naturally occurring system, the spacer sequence comprises a nucleotide sequence selected from the group consisting of SEQ ID NOs: 1729-1765, wherein the spacer sequence is capable of hybridizing with the human CD38 gene. In certain embodiments, when the system is delivered into a population of human cells ex vivo, the genomic sequence at the CD38 gene locus is edited in at least 1.5% of the cells.

In certain embodiments of the engineered, non-naturally occurring system, the spacer sequence comprises a nucleotide sequence selected from the group consisting of SEQ ID NOs: 1795-1796, wherein the spacer sequence is capable of hybridizing with the human CD3E gene. In certain embodiments, when the system is delivered into a population of human cells ex vivo, the genomic sequence at the CD3E gene locus is edited in at least 1.5% of the cells.

In certain embodiments of the engineered, non-naturally occurring system, the spacer sequence comprises a nucleotide sequence selected from the group consisting of SEQ ID NOs: 1518-1527, wherein the spacer sequence is capable of hybridizing with the human CD3G gene. In certain embodiments, when the system is delivered into a population of human cells ex vivo, the genomic sequence at the CD3G gene locus is edited in at least 1.5% of the cells.

In certain embodiments of the engineered, non-naturally occurring system, the spacer sequence comprises a nucleotide sequence selected from the group consisting of SEQ ID NOs: 1798, wherein the spacer sequence is capable of hybridizing with the human CD40LG gene. In certain embodiments, when the system is delivered into a population of human cells ex vivo, the genomic sequence at the CD40LG gene locus is edited in at least 1.5% of the cells.

In certain embodiments of the engineered, non-naturally occurring system, the spacer sequence comprises a nucleotide sequence selected from the group consisting of SEQ ID NOs: 985, 1039, 1116-1121, and 1466-1467, wherein the spacer sequence is capable of hybridizing with the human CD52 gene. In certain embodiments, when the system is delivered into a population of human cells ex vivo, the genomic sequence at the CD52 gene locus is edited in at least 1.5% of the cells.

In certain embodiments of the engineered, non-naturally occurring system, the spacer sequence comprises a nucleotide sequence selected from the group consisting of SEQ ID NOs: 1569-1578, wherein the spacer sequence is capable of hybridizing with the human CD58 gene. In certain embodiments, when the system is delivered into a population of human cells ex vivo, the genomic sequence at the CD58 gene locus is edited in at least 1.5% of the cells.

In certain embodiments of the engineered, non-naturally occurring system, the spacer sequence comprises a nucleotide sequence selected from the group consisting of SEQ ID NOs: 986, 1040-1041, 1122-1149, and 1303-1371, wherein the spacer sequence is capable of hybridizing with the human CIITA gene. In certain embodiments, when the system is delivered into a population of human cells ex vivo, the genomic sequence at the CIITA gene locus is edited in at least 1.5% of the cells.

In certain embodiments of the engineered, non-naturally occurring system, the spacer sequence comprises a nucleotide sequence selected from the group consisting of SEQ ID NOs: 1579-1588, wherein the spacer sequence is capable of hybridizing with the human COL17A1 gene. In certain embodiments, when the system is delivered into a population of human cells ex vivo, the genomic sequence at the COL 17Al gene locus is edited in at least 1.5% of the cells.

In certain embodiments of the engineered, non-naturally occurring system, the spacer sequence comprises a nucleotide sequence selected from the group consisting of SEQ ID NOs: 1792-1793, wherein the spacer sequence is capable of hybridizing with the human CSF1R gene. In certain embodiments, when the system is delivered into a population of human cells ex vivo, the genomic sequence at the CSF1R gene locus is edited in at least 1.5% of the cells.

In certain embodiments of the engineered, non-naturally occurring system, the spacer sequence comprises a nucleotide sequence selected from the group consisting of SEQ ID NOs: 1797, wherein the spacer sequence is capable of hybridizing with the human CSF2 gene. In certain embodiments, when the system is delivered into a population of human cells ex vivo, the genomic sequence at the CSF2 gene locus is edited in at least 1.5% of the cells.

In certain embodiments of the engineered, non-naturally occurring system, the spacer sequence comprises a nucleotide sequence selected from the group consisting of SEQ ID NOs: 112-152, wherein the spacer sequence is capable of hybridizing with the human CTLA4 gene. In certain embodiments, when the system is delivered into a population of human cells ex vivo, the genomic sequence at the CTLA4 gene locus is edited in at least 1.5% of the cells.

In certain embodiments of the engineered, non-naturally occurring system, the spacer sequence comprises a nucleotide sequence selected from the group consisting of SEQ ID NOs: 992-995, 1042-1045, 1150-1171, and 1433, wherein the spacer sequence is capable of hybridizing with the human DCK gene. In certain embodiments, when the system is delivered into a population of human cells ex vivo, the genomic sequence at the DCK gene locus is edited in at least 1.5% of the cells.

In certain embodiments of the engineered, non-naturally occurring system, the spacer sequence comprises a nucleotide sequence selected from the group consisting of SEQ ID NOs: 1589-1598, wherein the spacer sequence is capable of hybridizing with the human DEFB134 gene. In certain embodiments, when the system is delivered into a population of human cells ex vivo, the genomic sequence at the DEFB134 gene locus is edited in at least 1.5% of the cells.

In certain embodiments of the engineered, non-naturally occurring system, the spacer sequence comprises a nucleotide sequence selected from the group consisting of SEQ ID NOs: 1414-1416, wherein the spacer sequence is capable of hybridizing with the human DHODH gene. In certain embodiments, when the system is delivered into a population of human cells ex vivo, the genomic sequence at the DHODH gene locus is edited in at least 1.5% of the cells.

In certain embodiments of the engineered, non-naturally occurring system, the spacer sequence comprises a nucleotide sequence selected from the group consisting of SEQ ID NOs: 1659-1668, wherein the spacer sequence is capable of hybridizing with the human ERAPI gene. In certain embodiments, when the system is delivered into a population of human cells ex vivo, the genomic sequence at the ERAP1 gene locus is edited in at least 1.5% of the cells.

In certain embodiments of the engineered, non-naturally occurring system, the spacer sequence comprises a nucleotide sequence selected from the group consisting of SEQ ID NOs: 1669-1678, wherein the spacer sequence is capable of hybridizing with the human ERAP2 gene. In certain embodiments, when the system is delivered into a population of human cells ex vivo, the genomic sequence at the ERAP2 gene locus is edited in at least 1.5% of the cells.

In certain embodiments of the engineered, non-naturally occurring system, the spacer sequence comprises a nucleotide sequence selected from the group consisting of SEQ ID NOs: 996-1000, 1046-1059, 1172-1243, and 1781-1791, wherein the spacer sequence is capable of hybridizing with the human FAS gene. In certain embodiments, when the system is delivered into a population of human cells ex vivo, the genomic sequence at the FAS gene locus is edited in at least 1.5% of the cells.

In certain embodiments of the engineered, non-naturally occurring system, the spacer sequence comprises a nucleotide sequence selected from the group consisting of SEQ ID NOs: 1619-1621, wherein the spacer sequence is capable of hybridizing with the human mir-101-2 gene. In certain embodiments, when the system is delivered into a population of human cells ex vivo, the genomic sequence at the mir-101-2 gene locus is edited in at least 1.5% of the cells.

In certain embodiments of the engineered, non-naturally occurring system, the spacer sequence comprises a nucleotide sequence selected from the group consisting of SEQ ID NOs: 333-383 and 1244, wherein the spacer sequence is capable of hybridizing with the human HAVCR2 (TIM3) gene. In certain embodiments, when the system is delivered into a population of human cells ex vivo, the genomic sequence at the HAVCR2 (TIM3) gene locus is edited in at least 1.5% of the cells.

In certain embodiments of the engineered, non-naturally occurring system, the spacer sequence comprises a nucleotide sequence selected from the group consisting of SEQ ID NOs: 1679-1688, wherein the spacer sequence is capable of hybridizing with the human IFNGR1 gene. In certain embodiments, when the system is delivered into a population of human cells ex vivo, the genomic sequence at the IFNGR1 gene locus is edited in at least 1.5% of the cells.

In certain embodiments of the engineered, non-naturally occurring system, the spacer sequence comprises a nucleotide sequence selected from the group consisting of SEQ ID NOs: 1689-1698, wherein the spacer sequence is capable of hybridizing with the human IFNGR2 gene. In certain embodiments, when the system is delivered into a population of human cells ex vivo, the genomic sequence at the IFNGR2 gene locus is edited in at least 1.5% of the cells.

In certain embodiments of the engineered, non-naturally occurring system, the spacer sequence comprises a nucleotide sequence selected from the group consisting of SEQ ID NOs: 1391-1398, wherein the spacer sequence is capable of hybridizing with the human IL7R gene. In certain embodiments, when the system is delivered into a population of human cells ex vivo, the genomic sequence at the IL7R gene locus is edited in at least 1.5% of the cells.

In certain embodiments of the engineered, non-naturally occurring system, the spacer sequence comprises a nucleotide sequence selected from the group consisting of SEQ ID NOs: 1699-1718, wherein the spacer sequence is capable of hybridizing with the human JAKI gene. In certain embodiments, when the system is delivered into a population of human cells ex vivo, the genomic sequence at the JAKI gene locus is edited in at least 1.5% of the cells.

In certain embodiments of the engineered, non-naturally occurring system, the spacer sequence comprises a nucleotide sequence selected from the group consisting of SEQ ID NOs: 154-208, and 1245, wherein the spacer sequence is capable of hybridizing with the human LAG3 gene. In certain embodiments, when the system is delivered into a population of human cells ex vivo, the genomic sequence at the LAG3 gene locus is edited in at least 1.5% of the cells.

In certain embodiments of the engineered, non-naturally occurring system, the spacer sequence comprises a nucleotide sequence selected from the group consisting of SEQ ID NOs: 1399-1401, wherein the spacer sequence is capable of hybridizing with the human LCK1 gene. In certain embodiments, when the system is delivered into a population of human cells ex vivo, the genomic sequence at the LCK1 gene locus is edited in at least 1.5% of the cells.

In certain embodiments of the engineered, non-naturally occurring system, the spacer sequence comprises a nucleotide sequence selected from the group consisting of SEQ ID NOs: 1609-1618, wherein the spacer sequence is capable of hybridizing with the human MLANA gene. In certain embodiments, when the system is delivered into a population of human cells ex vivo, the genomic sequence at the MLANA gene locus is edited in at least 1.5% of the cells.

In certain embodiments of the engineered, non-naturally occurring system, the spacer sequence comprises a nucleotide sequence selected from the group consisting of SEQ ID NOs: 1427-1429, wherein the spacer sequence is capable of hybridizing with the human MVD gene. In certain embodiments, when the system is delivered into a population of human cells ex vivo, the genomic sequence at the MVD gene locus is edited in at least 1.5% of the cells.

In certain embodiments of the engineered, non-naturally occurring system, the spacer sequence comprises a nucleotide sequence selected from the group consisting of SEQ ID NOs: 209-238, wherein the spacer sequence is capable of hybridizing with the human PDCD1 (PD) gene. In certain embodiments, when the system is delivered into a population of human cells ex vivo, the genomic sequence at the PDCD1 (PD) gene locus is edited in at least 1.5% of the cells.

In certain embodiments of the engineered, non-naturally occurring system, the spacer sequence comprises a nucleotide sequence selected from the group consisting of SEQ ID NOs: 1402-1406, wherein the spacer sequence is capable of hybridizing with the human PLCG1 gene. In certain embodiments, when the system is delivered into a population of human cells ex vivo, the genomic sequence at the PLCG1 gene locus is edited in at least 1.5% of the cells.

In certain embodiments of the engineered, non-naturally occurring system, the spacer sequence comprises a nucleotide sequence selected from the group consisting of SEQ ID NOs: 1417-1426, wherein the spacer sequence is capable of hybridizing with the human PLK1 gene. In certain embodiments, when the system is delivered into a population of human cells ex vivo, the genomic sequence at the PLK1 gene locus is edited in at least 1.5% of the cells.

In certain embodiments of the engineered, non-naturally occurring system, the spacer sequence comprises a nucleotide sequence selected from the group consisting of SEQ ID NOs: 1529-1538, wherein the spacer sequence is capable of hybridizing with the human PSMB5 gene. In certain embodiments, when the system is delivered into a population of human cells ex vivo, the genomic sequence at the PSMB5 gene locus is edited in at least 1.5% of the cells.

In certain embodiments of the engineered, non-naturally occurring system, the spacer sequence comprises a nucleotide sequence selected from the group consisting of SEQ ID NOs: 1478-1487, wherein the spacer sequence is capable of hybridizing with the human PSMB8 gene. In certain embodiments, when the system is delivered into a population of human cells ex vivo, the genomic sequence at the PSMB8 gene locus is edited in at least 1.5% of the cells.

In certain embodiments of the engineered, non-naturally occurring system, the spacer sequence comprises a nucleotide sequence selected from the group consisting of SEQ ID NOs: 1468-1477, wherein the spacer sequence is capable of hybridizing with the human PSMB9 gene. In certain embodiments, when the system is delivered into a population of human cells ex vivo, the genomic sequence at the PSMB98 gene locus is edited in at least 1.5% of the cells.

In certain embodiments of the engineered, non-naturally occurring system, the spacer sequence comprises a nucleotide sequence selected from the group consisting of SEQ ID NOs: 1638-1647, wherein the spacer sequence is capable of hybridizing with the human PTCD2 gene. In certain embodiments, when the system is delivered into a population of human cells ex vivo, the genomic sequence at the PTCD2 gene locus is edited in at least 1.5% of the cells.

In certain embodiments of the engineered, non-naturally occurring system, the spacer sequence comprises a nucleotide sequence selected from the group consisting of SEQ ID NOs: 239-241, wherein the spacer sequence is capable of hybridizing with the human PTPNI gene. In certain embodiments, when the system is delivered into a population of human cells ex vivo, the genomic sequence at the PTPNI gene locus is edited in at least 1.5% of the cells.

In certain embodiments of the engineered, non-naturally occurring system, the spacer sequence comprises a nucleotide sequence selected from the group consisting of SEQ ID NOs: 242-248 wherein the spacer sequence is capable of hybridizing with the human PTPN11 gene. In certain embodiments, when the system is delivered into a population of human cells ex vivo, the genomic sequence at the PTPN11 gene locus is edited in at least 1.5% of the cells.

In certain embodiments of the engineered, non-naturally occurring system, the spacer sequence comprises a nucleotide sequence selected from the group consisting of SEQ ID NOs: 249-301 and 1246-1248, wherein the spacer sequence is capable of hybridizing with the human PTPN6 gene. In certain embodiments, when the system is delivered into a population of human cells ex vivo, the genomic sequence at the PTPN6 gene locus is edited in at least 1.5% of the cells.

In certain embodiments of the engineered, non-naturally occurring system, the spacer sequence comprises a nucleotide sequence selected from the group consisting of SEQ ID NOs: 1488-1497, wherein the spacer sequence is capable of hybridizing with the human RFX5 gene. In certain embodiments, when the system is delivered into a population of human cells ex vivo, the genomic sequence at the RFX5 gene locus is edited in at least 1.5% of the cells.

In certain embodiments of the engineered, non-naturally occurring system, the spacer sequence comprises a nucleotide sequence selected from the group consisting of SEQ ID NOs: 1498-1507, wherein the spacer sequence is capable of hybridizing with the human RFXAP gene. In certain embodiments, when the system is delivered into a population of human cells ex vivo, the genomic sequence at the RFXAP gene locus is edited in at least 1.5% of the cells.

In certain embodiments of the engineered, non-naturally occurring system, the spacer sequence comprises a nucleotide sequence selected from the group consisting of SEQ ID NOs: 1628-1637, wherein the spacer sequence is capable of hybridizing with the human RPL23 gene. In certain embodiments, when the system is delivered into a population of human cells ex vivo, the genomic sequence at the RPL23 gene locus is edited in at least 1.5% of the cells.

In certain embodiments of the engineered, non-naturally occurring system, the spacer sequence comprises a nucleotide sequence selected from the group consisting of SEQ ID NOs: 1508-1517, wherein the spacer sequence is capable of hybridizing with the human RFXANK gene. In certain embodiments, when the system is delivered into a population of human cells ex vivo, the genomic sequence at the RFXANK gene locus is edited in at least 1.5% of the cells.

In certain embodiments of the engineered, non-naturally occurring system, the spacer sequence comprises a nucleotide sequence selected from the group consisting of SEQ ID NOs: 1622-1627, wherein the spacer sequence is capable of hybridizing with the human SOX10 gene. In certain embodiments, when the system is delivered into a population of human cells ex vivo, the genomic sequence at the SOX10 gene locus is edited in at least 1.5% of the cells.

In certain embodiments of the engineered, non-naturally occurring system, the spacer sequence comprises a nucleotide sequence selected from the group consisting of SEQ ID NOs: 1648-1658, wherein the spacer sequence is capable of hybridizing with the human SRP54 gene. In certain embodiments, when the system is delivered into a population of human cells ex vivo, the genomic sequence at the SRP54 gene locus is edited in at least 1.5% of the cells.

In certain embodiments of the engineered, non-naturally occurring system, the spacer sequence comprises a nucleotide sequence selected from the group consisting of SEQ ID NOs: 1719-1728, wherein the spacer sequence is capable of hybridizing with the human STAT1 gene. In certain embodiments, when the system is delivered into a population of human cells ex vivo, the genomic sequence at the STAT1 gene locus is edited in at least 1.5% of the cells.

In certain embodiments of the engineered, non-naturally occurring system, the spacer sequence comprises a nucleotide sequence selected from the group consisting of SEQ ID NOs: 1436-1445, wherein the spacer sequence is capable of hybridizing with the human TAP1 gene. In certain embodiments, when the system is delivered into a population of human cells ex vivo, the genomic sequence at the TAP1 gene locus is edited in at least 1.5% of the cells.

In certain embodiments of the engineered, non-naturally occurring system, the spacer sequence comprises a nucleotide sequence selected from the group consisting of SEQ ID NOs: 1446-1455, wherein the spacer sequence is capable of hybridizing with the human TAP2 gene. In certain embodiments, when the system is delivered into a population of human cells ex vivo, the genomic sequence at the TAP2 gene locus is edited in at least 1.5% of the cells.

In certain embodiments of the engineered, non-naturally occurring system, the spacer sequence comprises a nucleotide sequence selected from the group consisting of SEQ ID NOs: 1456-1465, wherein the spacer sequence is capable of hybridizing with the human TAPBP gene. In certain embodiments, when the system is delivered into a population of human cells ex vivo, the genomic sequence at the TAPBP gene locus is edited in at least 1.5% of the cells.

In certain embodiments of the engineered, non-naturally occurring system, the spacer sequence comprises a nucleotide sequence selected from the group consisting of SEQ ID NOs: 1766-1780, wherein the spacer sequence is capable of hybridizing with the human TGFBR2 gene. In certain embodiments, when the system is delivered into a population of human cells ex vivo, the genomic sequence at the TGFBR2 gene locus is edited in at least 1.5% of the cells.

In certain embodiments of the engineered, non-naturally occurring system, the spacer sequence comprises a nucleotide sequence selected from the group consisting of SEQ ID NOs: 302-332, wherein the spacer sequence is capable of hybridizing with the human TIGIT gene. In certain embodiments, when the system is delivered into a population of human cells ex vivo, the genomic sequence at the TIGIT gene locus is edited in at least 1.5% of the cells.

In certain embodiments of the engineered, non-naturally occurring system, the spacer sequence comprises a nucleotide sequence selected from the group consisting of SEQ ID NOs: 1001-1023, 1060-1083, 1249-1283, and 1434-1435, wherein the spacer sequence is capable of hybridizing with the human TRAC gene. In certain embodiments, when the system is delivered into a population of human cells ex vivo, the genomic sequence at the TRAC gene locus is edited in at least 1.5% of the cells.

In certain embodiments of the engineered, non-naturally occurring system, the spacer sequence comprises a nucleotide sequence selected from the group consisting of SEQ ID NOs: 1372-1373, wherein the spacer sequence is capable of hybridizing with the human TRBC1+2 gene. In certain embodiments, when the system is delivered into a population of human cells ex vivo, the genomic sequence at the TRBC1+2 gene locus is edited in at least 1.5% of the cells.

In certain embodiments of the engineered, non-naturally occurring system, the spacer sequence comprises a nucleotide sequence selected from the group consisting of SEQ ID NOs: 1794, wherein the spacer sequence is capable of hybridizing with the human TRBC1 gene. In certain embodiments, when the system is delivered into a population of human cells ex vivo, the genomic sequence at the TRBC1 gene locus is edited in at least 1.5% of the cells.

In certain embodiments of the engineered, non-naturally occurring system, the spacer sequence comprises a nucleotide sequence selected from the group consisting of SEQ ID NOs: 1374-1387, wherein the spacer sequence is capable of hybridizing with the human TRBC2 gene. In certain embodiments, when the system is delivered into a population of human cells ex vivo, the genomic sequence at the TRBC2 gene locus is edited in at least 1.5% of the cells.

In certain embodiments of the engineered, non-naturally occurring system, the spacer sequence comprises a nucleotide sequence selected from the group consisting of SEQ ID NOS: 1430-1432, wherein the spacer sequence is capable of hybridizing with the human TUBB gene. In certain embodiments, when the system is delivered into a population of human cells ex vivo, the genomic sequence at the TUBB gene locus is edited in at least 1.5% of the cells.

In certain embodiments of the engineered, non-naturally occurring system, the spacer sequence comprises a nucleotide sequence selected from the group consisting of SEQ ID NOs: 1599-1608, wherein the spacer sequence is capable of hybridizing with the human TWF1. In certain embodiments, when the system is delivered into a population of human cells ex vivo, the genomic sequence at the TWFI gene locus is edited in at least 1.5% of the cells.

In certain embodiments of the engineered, non-naturally occurring system, the spacer sequence comprises a nucleotide sequence selected from the group consisting of SEQ ID NOs: 1407-1413, wherein the spacer sequence is capable of hybridizing with the human U6 gene. In certain embodiments, when the system is delivered into a population of human cells ex vivo, the genomic sequence at the U6 gene locus is edited in at least 1.5% of the cells.

In certain embodiments of the engineered, non-naturally occurring system, genomic mutations are detected in no more than 2% of the cells at any off-target loci by CIRCLE-Seq. In certain embodiments, genomic mutations are detected in no more than 1% of the cells at any off-target loci by CIRCLE-Seq.

Table 10 shows an exemplary list of the spacer sequences of tested guide nucleic acids. In particular, Table 7 lists the spacer sequences of guide nucleic acids that showed the best editing efficiency for each target gene. Table 8 lists the spacer sequences of guide nucleic acids that showed at least 10% editing efficiency. Table 9 lists the spacer sequences of guide nucleic acids that showed at least 1.5% and lower than 10% editing efficiency.

In certain embodiments, a guide nucleic acid of the present invention is capable of binding the genomic locus of the corresponding target gene in the human genome. In certain embodiments, a guide nucleic acid of the present invention, alone or in combination with a modulator nucleic acid, is capable of directing a Cas protein to the genomic locus of the corresponding target gene in the human genome. In certain embodiments, a guide nucleic acid of the present invention, alone or in combination with a modulator nucleic acid, is capable of directing a Cas nuclease to the genomic locus of the corresponding target gene in the human genome, thereby resulting in cleavage of the genomic DNA at the genomic locus.

TABLE 5
Spacer sequences

| Target Gene | Name | SEQ ID NO | PAM | Spacer Sequence |
|---|---|---|---|---|
| CD247 | crCD247_1 | 86 | TTTC | ACCGCGGCCATCCTGCAGGCA |
| CD247 | crCD247_2 | 87 | TTTC | TGAGGGAAAGGACAAGATGAA |
| CD247 | crCD247_3 | 88 | TTTG | GGATCCAGCAGGCCAAAGCTC |
| CD247 | crCD247_4 | 89 | TTTC | CTAGCAGAGAAGGAAGAACCC |
| CD247 | crCD247_5 | 90 | TTTC | TGTGTTGCAGTTCAGCAGGAG |
| CD247 | crCD247_6 | 91 | CTTC | CTGAGGGTTCTTCCTTCTCTG |
| CD247 | crCD247_7 | 92 | CTTC | CCGTTGTCTTTCCTAGCAGAG |
| CD247 | crCD247_8 | 93 | TTTC | TGCAGTTCCTGCAGAAGAGGG |
| CD247 | crCD247_9 | 94 | CTTC | TGCAGGAACTGCAGAAAGATA |
| CD247 | crCD247_10 | 95 | TTTC | ATCCCAATCTCACTGTAGGCC |
| CD247 | crCD247_11 | 96 | CTTT | CATCCCAATCTCACTGTAGGC |
| CD247 | crCD247_12 | 97 | TTTT | CTCATTTCACTCCCAAACAAC |
| CD247 | crCD247_13 | 98 | TTTC | TCATTTCACTCCCAAACAACC |
| CD247 | crCD247_14 | 99 | TTTC | ACTCCCAAACAACCAGCGCCG |
| CD247 | crCD247_15 | 100 | CTTA | CGTTATAGAGCTGGTTCTGGC |
| CD247 | crCD247_16 | 101 | TTTG | TTTTCTGATTTGCTTTCACGC |
| CD247 | crCD247_17 | 102 | TTTC | TGATTTGCTTTCACGCCAGGG |
| CD247 | crCD247_18 | 103 | TTTG | CTTTCACGCCAGGGTCTCAGT |
| CD247 | crCD247_19 | 104 | TTTC | ACGCCAGGGTCTCAGTACAGC |
| CD247 | crCD247_20 | 105 | TTTC | CGGAGGGTCTACGGCGAGGCT |
| CD247 | crCD247_21 | 106 | TTTC | TTATCTGTTATAGGAGCTCAA |
| CD247 | crCD247_22 | 107 | CTTA | TCTGTTATAGGAGCTCAATCT |
| CD247 | crCD247_23 | 108 | CTTG | TCCAAAACATCGTACTCCTCT |
| CD247 | crCD247_24 | 109 | TTTC | CCCCCATCTCAGGGTCCCGGC |
| CD247 | crCD247_25 | 110 | TTTG | GACAAGAGACGTGGCCGGGAC |
| CD247 | crCD247_26 | 111 | TTTC | TCTCCCTCTAACGTCTTCCCG |
| CTLA4 | crCTLA4_1 | 112 | TTTG | CCTGGAGATGCATACTCACAC |
| CTLA4 | crCTLA4_2 | 113 | TTTG | CAGAAGACAGGGATGAAGAGA |
| CTLA4 | crCTLA4_3 | 114 | TTTC | CACTGGAGGTGCCCGTGCAGA |
| CTLA4 | crCTLA4_4 | 115 | TTTG | TGTGTGAGTATGCATCTCCAG |
| CTLA4 | crCTLA4_5 | 116 | TTTC | AGCGGCACAAGGCTCAGCTGA |
| CTLA4 | crCTLA4_6 | 117 | CTTG | TGCCGCTGAAATCCAAGGCAA |
| CTLA4 | crCTLA4_7 | 118 | CTTT | TCCATGCTAGCAATGCACGTG |
| CTLA4 | crCTLA4_8 | 119 | TTTT | CCATGCTAGCAATGCACGTGG |
| CTLA4 | crCTLA4_9 | 120 | CTTT | GTGTGTGAGTATGCATCTCCA |
| CTLA4 | crCTLA4_10 | 121 | CTTT | GCCTGGAGATGCATACTCACA |
| CTLA4 | crCTLA4_11 | 122 | CTTC | GGCAGGCTGACAGCCAGGTGA |
| CTLA4 | crCTLA4_12 | 123 | CTTC | AGTCACCTGGCTGTCAGCCTG |
| CTLA4 | crCTLA4_13 | 124 | CTTC | CTAGATGATTCCATCTGCACG |
| CTLA4 | crCTLA4_14 | 125 | CTTG | CCTTGGATTTCAGCGGCACAA |
| CTLA4 | crCTLA4_15 | 126 | CTTG | ATTTCCACTGGAGGTGCCCGT |
| CTLA4 | crCTLA4_16 | 127 | CTTG | GATAGTGAGGTTCACTTGATT |
| CTLA4 | crCTLA4_17 | 128 | CTTG | CAGATGTAGAGTCCCGTGTCC |
| CTLA4 | crCTLA4_18 | 129 | TTTG | CTCACCAATTACATAAATCTG |
| CTLA4 | crCTLA4_19 | 130 | CTTT | GCTCACCAATTACATAAATCT |
| CTLA4 | crCTLA4_20 | 131 | CTTT | GTTTTCTGTTGCAGATCCAGA |
| CTLA4 | crCTLA4_21 | 132 | TTTG | TTTTCTGTTGCAGATCCAGAA |
| CTLA4 | crCTLA4_22 | 133 | TTTT | CTGTTGCAGATCCAGAACCGT |
| CTLA4 | crCTLA4_23 | 134 | CTTC | CTCCTCTGGATCCTTGCAGCA |
| CTLA4 | crCTLA4_24 | 135 | CTTG | CAGCAGTTAGTTCGGGGTTGT |
| CTLA4 | crCTLA4_25 | 136 | CTTG | GATTTCAGCGGCACAAGGCTC |
| CTLA4 | crCTLA4_26 | 137 | TTTT | TTTATAGCTTTCTCCTCACAG |
| CTLA4 | crCTLA4_27 | 138 | CTTT | CTCCTCACAGCTGTTTCTTTG |
| CTLA4 | crCTLA4_28 | 139 | TTTC | TCCTCACAGCTGTTTCTTTGA |
| CTLA4 | crCTLA4_29 | 140 | TTTT | GCTCAAAGAAACAGCTGTGAG |
| CTLA4 | crCTLA4_30 | 141 | TTTC | TTTTTGTGTTTGACAGCTAAA |
| CTLA4 | crCTLA4_31 | 142 | TTTT | TGTGTTTGACAGCTAAAGAAA |
| CTLA4 | crCTLA4_32 | 143 | TTTG | ACAGCTAAAGAAAAGAAGCCC |
| CTLA4 | crCTLA4_33 | 144 | TTTT | CACATAGACCCCTGTTGTAAG |
| CTLA4 | crCTLA4_34 | 145 | TTTT | CACATTCTGGCTCTGTTGGGG |
| CTLA4 | crCTLA4_35 | 146 | CTTT | TCACATTCTGGCTCTGTTGGG |
| CTLA4 | crCTLA4_36 | 147 | TTTC | AGCCTTATTTTATTCCCATCA |
| CTLA4 | crCTLA4_37 | 148 | TTTC | TCAATTGATGGGAATAAAATA |
| CTLA4 | crCTLA4_38 | 149 | TTTT | TTCTTCTCTTCATCCCTGTCT |
| CTLA4 | crCTLA4_39 | 150 | CTTT | GCAGAAGACAGGGATGAAGAG |
| CTLA4 | crCTLA4_40 | 151 | CTTT | GGCTTTTCCATGCTAGCAATG |
| CTLA4 | crCTLA4_41 | 152 | TTTG | GCTTTTCCATGCTAGCAATGC |
| LAG3 | crLAG3_1 | 153 | TTTG | GGGTGCATACCTGTCTGGCTG |
| LAG3 | crLAG3_2 | 154 | TTTG | GGTCACCTGGATCCCTGGGGA |
| LAG3 | crLAG3_3 | 155 | TTTC | TCAGGACCTTGGCTGGAGGCA |
| LAG3 | crLAG3_4 | 156 | TTTC | CCAGCCTTGGCAATGCCAGCT |
| LAG3 | crLAG3_5 | 157 | TTTG | TGAGGTGACTCCAGTATCTGG |
| LAG3 | crLAG3_6 | 158 | CTTG | CTGTTTCTGCAGCCGCTTTGG |
| LAG3 | crLAG3_7 | 159 | CTTG | CACAGTGACTGCCAGCCCCCC |
| LAG3 | crLAG3_8 | 160 | TTTT | GAACTGCTCCTTCAGCCGCCC |
| LAG3 | crLAG3_9 | 161 | CTTC | AGCCGCCCTGACCGCCCAGCC |
| LAG3 | crLAG3_10 | 162 | TTTC | CGCTAAGTGGTGATGGGGGGA |
| LAG3 | crLAG3_11 | 163 | CTTT | CCGCTAAGTGGTGATGGGGGG |
| LAG3 | crLAG3_12 | 164 | CTTA | GCGGAAAGCTTCCTCTTCCTG |
| LAG3 | crLAG3_13 | 165 | CTTG | GGGCAGGAAGAGGAAGCTTTC |
| LAG3 | crLAG3_14 | 166 | CTTC | CTCTTCCTGCCCCAAGTCAGC |
| LAG3 | crLAG3_15 | 167 | CTTC | AACGTCTCCATCATGTATAAC |
| LAG3 | crLAG3_16 | 168 | TTTT | CTTTTCTCTTCAGGTCTGGAG |
| LAG3 | crLAG3_17 | 169 | TTTC | TGCAGCCGCTTTGGGTGGCTC |
| LAG3 | crLAG3_18 | 170 | TTTT | CTCTTCAGGTCTGGAGCCCCC |
| LAG3 | crLAG3_19 | 171 | CTTG | ACAGTGTACGCTGGAGCAGGT |
| LAG3 | crLAG3_20 | 172 | CTTG | GCAGTGAGGAAAGACCGGGTC |
| LAG3 | crLAG3_21 | 173 | TTTC | CTCACTGCCAAGTGGACTCCT |
| LAG3 | crLAG3_22 | 174 | CTTT | ACCCTTCGACTAGAGGATGTG |
| LAG3 | crLAG3_23 | 175 | TTTA | CCCTTCGACTAGAGGATGTGA |
| LAG3 | crLAG3_24 | 176 | CTTC | GACTAGAGGATGTGAGCCAGG |
| LAG3 | crLAG3_25 | 177 | TTTC | CCACCTGAGGCTGACCTGTGA |
| LAG3 | crLAG3_26 | 178 | CTTT | CCCACCTGAGGCTGACCTGTG |
| LAG3 | crLAG3_27 | 179 | CTTC | TACTCTTTTCAGTGACTCCCA |
| LAG3 | crLAG3_28 | 180 | TTTT | ACCTGGAGCCACCCAAAGCGG |
| LAG3 | crLAG3_29 | 181 | TTTT | CAGTGACTCCCAAATCCTTTG |
| LAG3 | crLAG3_30 | 182 | CTTC | CCCAGGGATCCAGGTGACCCA |
| LAG3 | crLAG3_31 | 183 | CTTT | GGGTCACCTGGATCCCTGGGG |
| LAG3 | crLAG3_32 | 184 | CTTT | GTGAGGTGACTCCAGTATCTG |
| LAG3 | crLAG3_33 | 185 | CTTT | GTGTGGAGCTCTCTGGACACC |
| LAG3 | crLAG3_34 | 186 | TTTG | TGTGGAGCTCTCTGGACACCC |
| LAG3 | crLAG3_35 | 187 | CTTG | GCTGGAGGCACAGGAGGCCCA |
| LAG3 | crLAG3_36 | 188 | TTTT | GCTCACCTAGTGAAGCCTCTC |
| LAG3 | crLAG3_37 | 189 | CTTT | CCCAGCCTTGGCAATGCCAGC |
| LAG3 | crLAG3_38 | 190 | CTTG | GCAATGCCAGCTGTACCAGGG |
| LAG3 | crLAG3_39 | 191 | CTTC | TTGGAGCAGCAGTGTACTTCA |
| LAG3 | crLAG3_40 | 192 | CTTC | ACAGAGCTGTCTAGCCCAGGT |
| LAG3 | crLAG3_41 | 193 | CTTT | CTCCATAGGTGCCCAACGCTC |
| LAG3 | crLAG3_42 | 194 | TTTC | TCCATAGGTGCCCAACGCTCT |
| LAG3 | crLAG3_43 | 195 | TTTC | TCATCCTTGGTGTCCTTTCTC |
| LAG3 | crLAG3_44 | 196 | CTTG | GTGTCCTTTCTCTGCTCCTTT |
| LAG3 | crLAG3_45 | 197 | CTTT | CTCTGCTCCTTTTGGTGACTG |
| LAG3 | crLAG3_46 | 198 | CTTC | TGCGAAGAGCAGGGGTCACTT |
| LAG3 | crLAG3_47 | 199 | CTTT | TGGTGACTGGAGCCTTTGGCT |
| LAG3 | crLAG3_48 | 200 | TTTT | GGTGACTGGAGCCTTTGGCTT |
| LAG3 | crLAG3_49 | 201 | CTTT | GGCTTTCACCTTTGGAGAAGA |
| LAG3 | crLAG3_50 | 202 | TTTG | GCTTTCACCTTTGGAGAAGAC |
| LAG3 | crLAG3_51 | 203 | CTTG | CTCTAAGGCAGAAAATCGTCT |
| LAG3 | crLAG3_52 | 204 | TTTT | CTGCCTTAGAGCAAGGGATTC |
| LAG3 | crLAG3_53 | 205 | CTTA | GAGCAAGGGATTCACCCTCCG |
| LAG3 | crLAG3_54 | 206 | TTTC | CCGCCCAGTGGCCCGCCCGCT |
| LAG3 | crLAG3_55 | 207 | CTTC | TCGCTATGGCTGCGCCCAGCC |
| LAG3 | crLAG3_56 | 208 | TTTA | TCCTTGCACAGTGACTGCCAG |
| PDCD1 | crPDCD1_1 | 209 | TTTA | GCACGAAGCTCTCCGATGTGT |
| PDCD1 | crPDCD1_2 | 210 | TTTC | TCTGCAGGGACAATAGGAGCC |
| PDCD1 | crPDCD1_3 | 211 | TTTC | CAGTGGCGAGAGAAGACCCCG |
| PDCD1 | crPDCD1_4 | 212 | TTTC | CTAGCGGAATGGGCACCTCAT |
| PDCD1 | crPDCD1_5 | 213 | CTTC | GTGCTAAACTGGTACCGCATG |
| PDCD1 | crPDCD1_6 | 214 | CTTC | AACCTGACCTGGGACAGTTTC |
| PDCD1 | crPDCD1_7 | 215 | CTTG | TCCGTCTGGTTGCTGGGGCTC |
| PDCD1 | crPDCD1_8 | 216 | CTTC | CCCGAGGACCGCAGCCAGCCC |
| PDCD1 | crPDCD1_9 | 217 | CTTC | CGTGTCACACAACTGCCCAAC |
| PDCD1 | crPDCD1_10 | 218 | CTTC | CACATGAGCGTGGTCAGGGCC |
| PDCD1 | crPDCD1_11 | 219 | CTTT | GATCTGCGCCTTGGGGGCCAG |
| PDCD1 | crPDCD1_12 | 220 | TTTG | ATCTGCGCCTTGGGGGCCAGG |
| PDCD1 | crPDCD1_13 | 221 | CTTG | GGGGCCAGGGAGATGGCCCCA |
| PDCD1 | crPDCD1_14 | 222 | CTTT | GTGCCCTTCCAGAGAGAAGGG |
| PDCD1 | crPDCD1_15 | 223 | TTTG | TGCCCTTCCAGAGAGAAGGGC |
| PDCD1 | crPDCD1_16 | 224 | TTTC | CCTTCCGCTCACCTCCGCCTG |
| PDCD1 | crPDCD1_17 | 225 | CTTC | CAGAGAGAAGGGCAGAAGTGC |
| PDCD1 | crPDCD1_18 | 226 | CTTC | TGCCCTTCTCTCTGGAAGGGC |
| PDCD1 | crPDCD1_19 | 227 | TTTG | GAACTGGCCGGCTGGCCTGGG |
| PDCD1 | crPDCD1_20 | 228 | CTTT | CTCCTCAAAGAAGGAGGACCC |
| PDCD1 | crPDCD1_21 | 229 | TTTC | TCCTCAAAGAAGGAGGACCCC |
| PDCD1 | crPDCD1_22 | 230 | CTTC | TCTCGCCACTGGAAATCCAGC |
| PDCD1 | crPDCD1_23 | 231 | CTTT | CCTAGCGGAATGGGCACCTCA |
| PDCD1 | crPDCD1_24 | 232 | CTTC | CGCTCACCTCCGCCTGAGCAG |
| PDCD1 | crPDCD1_25 | 233 | CTTG | GCCCCTCTGACCGGCTTCCTT |
| PDCD1 | crPDCD1_26 | 234 | CTTC | TCCACTGCTCAGGCGGAGGTG |
| PDCD1 | crPDCD1_27 | 235 | CTTC | TCCCCAGCCCTGCTCGTGGTG |
| PDCD1 | crPDCD1_28 | 236 | CTTC | GGTCACCACGAGCAGGGCTGG |
| PDCD1 | crPDCD1_29 | 237 | CTTC | ACCTGCAGCTTCTCCAACACA |
| PDCD1 | crPDCD1_30 | 238 | CTTC | TCCAACACATCGGAGAGCTTC |
| PTPN1 | crPTPN1_1 | 239 | TTTA | CCTGACAGCGAATCATAACAT |
| PTPN1 | crPTPN1_2 | 240 | TTTC | ATTCCAACTTACCTAACGGAA |
| PTPN1 | crPTPN1_3 | 241 | TTTC | TGTGCGCACTGGTGATGACAA |
| PTPN11 | crPTPN11_4 | 242 | TTTC | CAATCTGCTCACCTGCTTGAG |
| PTPN11 | crPTPN11_5 | 243 | TTTC | TTCTAGTTGATCATACCAGGG |
| PTPN11 | crPTPN11_6 | 244 | TTTA | ATAACTTACCTCAAATTCTTC |
| PTPN11 | crPTPN11_7 | 245 | CTTA | CCTAACGGAAAGTGTGAAGTC |
| PTPN11 | crPTPN11_8 | 246 | TTTC | CAGACACTACAACAACAGGAG |
| PTPN11 | crPTPN11_9 | 247 | TTTA | GGTGGTTTCATGGACATCTCT |
| PTPN11 | crPTPN11_10 | 248 | TTTC | CCAGAGAGATGTCCATGAAAC |
| PTPN6 | crPTPN6_1 | 249 | TTTC | TATGACCTGTATGGAGGGGAG |
| PTPN6 | crPTPN6_2 | 250 | TTTG | CGACTCTGACAGAGCTGGTGG |
| PTPN6 | crPTPN6_3 | 251 | TTTG | CAGAAGCAGGAGGTGAAGAAC |
| PTPN6 | crPTPN6_4 | 252 | TTTG | ACTGCCCCCCACCCAGGCCTG |
| PTPN6 | crPTPN6_5 | 253 | CTTA | TGGGCCCTACTCTGTGACCAA |
| PTPN6 | crPTPN6_6 | 254 | TTTC | ACCGAGACCTCAGTGGGCTGG |
| PTPN6 | crPTPN6_7 | 255 | CTTC | TCTAGGTGGTACCATGGCCAC |
| PTPN6 | crPTPN6_8 | 256 | CTTG | GCCTGCAGCAGCGTCTCTGCC |
| PTPN6 | crPTPN6_9 | 257 | TTTC | TTGTGCGTGAGAGCCTCAGCC |
| PTPN6 | crPTPN6_10 | 258 | CTTC | GTGCTTTCTGTGCTCAGTGAC |
| PTPN6 | crPTPN6_11 | 259 | CTTG | GGCTGGTCACTGAGCACAGAA |
| PTPN6 | crPTPN6_12 | 260 | CTTT | CTGTGCTCAGTGACCAGCCCA |
| PTPN6 | crPTPN6_13 | 261 | TTTC | TGTGCTCAGTGACCAGCCCAA |
| PTPN6 | crPTPN6_14 | 262 | CTTG | ATGTGGGTGACCCTGAGCGGG |
| PTPN6 | crPTPN6_15 | 263 | CTTA | CCTCGCACATGACCTTGATGT |
| PTPN6 | crPTPN6_16 | 264 | TTTG | GCTCCCCCCAGGGTGGACGCT |
| PTPN6 | crPTPN6_17 | 265 | CTTG | AGCAGGGTCTCTGCATCCAGC |
| PTPN6 | crPTPN6_18 | 266 | TTTG | GAGACCTTCGACAGCCTCACG |
| PTPN6 | crPTPN6_19 | 267 | CTTC | GACAGCCTCACGGACCTGGTG |
| PTPN6 | crPTPN6_20 | 268 | TTTC | AAGAAGACGGGGATTGAGGAG |
| PTPN6 | crPTPN6_21 | 269 | CTTC | TTGTTCAGTTCCAACACTCGG |
| PTPN6 | crPTPN6_22 | 270 | CTTG | GCTGTATCCTCGGACTCCTGC |
| PTPN6 | crPTPN6_23 | 271 | TTTC | CCCACCCACATCTCAGAGTTT |
| PTPN6 | CrPTPN6_24 | 272 | CTTC | CAGACGCTGGTGCAAGTTCTT |
| PTPN6 | crPTPN6_25 | 273 | CTTG | CACCAGCGTCTGGAAGGGCAG |
| PTPN6 | crPTPN6_26 | 274 | CTTG | TTCTCTGGCCGCTGCCCTTCC |
| PTPN6 | crPTPN6_27 | 275 | CTTG | ATGTAGTTGGCATTGATGTAG |
| PTPN6 | crPTPN6_28 | 276 | CTTG | CGTCCAGAACCAGCTGCTAGG |
| PTPN6 | crPTPN6_29 | 277 | CTTC | TGGCAGATGGCGTGGCAGGAG |
| PTPN6 | crPTPN6_30 | 278 | TTTC | TCCACCTCTCGGGTGGTCATG |
| PTPN6 | crPTPN6_31 | 279 | CTTT | CTCCACCTCTCGGGTGGTCAT |
| PTPN6 | crPTPN6_32 | 280 | CTTT | CCAGAACAAATGCGTCCCATA |
| PTPN6 | crPTPN6_33 | 281 | TTTC | CAGAACAAATGCGTCCCATAC |
| PTPN6 | crPTPN6_34 | 282 | TTTG | TATTCGGTTGTGTCATGCTCC |
| PTPN6 | crPTPN6_35 | 283 | CTTA | CAGGTCTCCCCGCTGGACAAT |
| PTPN6 | crPTPN6_36 | 284 | CTTC | CTGGCTCGGCCCAGTCGCAAG |
| PTPN6 | crPTPN6_37 | 285 | CTTA | GGGAGACCTGATTCGGGAGAT |
| PTPN6 | crPTPN6_38 | 286 | CTTC | CTGGACCAGATCAACCAGCGG |
| PTPN6 | crPTPN6_39 | 287 | TTTC | CTGCCGCTGGTTGATCTGGTC |
| PTPN6 | crPTPN6_40 | 288 | CTTT | CCTGCCGCTGGTTGATCTGGT |
| PTPN6 | crPTPN6_41 | 289 | CTTG | GTGGAGATGTTCTCCATGAGC |
| PTPN6 | crPTPN6_42 | 290 | CTTG | TACTGCGCCTCCGTCTGCACC |
| PTPN6 | crPTPN6_43 | 291 | TTTC | AATGAACTGGGCGATGGCCAC |
| PTPN6 | crPTPN6_44 | 292 | CTTC | TTCTTAGTGGTTTCAATGAAC |
| PTPN6 | crPTPN6_45 | 293 | CTTC | TCCCCTCCATACAGGTCATAG |
| PTPN6 | crPTPN6_46 | 294 | CTTG | GAGTCTAGTGCAGGGACCGTG |
| PTPN6 | crPTPN6_47 | 295 | CTTG | CCCCCCTGCACCCGGCTGCAG |
| PTPN6 | crPTPN6_48 | 296 | CTTG | TGTCTGCAGCCGGGTGCAGGG |
| PTPN6 | crPTPN6_49 | 297 | TTTC | TCCTCCCTCTTGTTCTTAGTG |
| PTPN6 | crPTPN6_50 | 298 | CTTT | CTCCTCCCTCTTGTTCTTAGT |
| PTPN6 | crPTPN6_51 | 299 | CTTC | TTCACTTTCTCCTCCCTCTTG |
| PTPN6 | crPTPN6_52 | 300 | CTTG | AGGTGGATGATGGTGCCGTCG |
| PTPN6 | crPTPN6_53 | 301 | CTTC | CCTGACGCTGCCTTCTCTAGG |
| TIGIT | CrTIGIT_1 | 302 | TTTC | AGGCCTTACCTGAGGCGAGGG |
| TIGIT | crTIGIT_2 | 303 | TTTT | GTCCTCCCTCTAGTGGCTGAG |
| TIGIT | CrTIGIT_3 | 304 | CTTG | GGGTGGCACATCTCCCCATCC |
| TIGIT | crTIGIT_4 | 305 | TTTC | TGCAGAGAAAGGTGGCTCTAT |
| TIGIT | CrTIGIT_5 | 306 | TTTG | TAATGCTGACTTGGGGTGGCA |
| TIGIT | crTIGIT_6 | 307 | CTTA | CCTGAGGCGAGGGGAGCCTGC |
| TIGIT | crTIGIT_7 | 308 | CTTG | AAGGATGGGGAGATGTGCCAC |
| TIGIT | CrTIGIT_8 | 309 | CTTC | AAGGATCGAGTGGCCCCAGGT |
| TIGIT | CrTIGIT_9 | 310 | CTTC | TGCATCTATCACACCTACCCT |
| TIGIT | CrTIGIT_10 | 311 | TTTC | TAGGACCTCCAGGAAGATTCT |
| TIGIT | crTIGIT_11 | 312 | CTTT | CTAGGACCTCCAGGAAGATTC |
| TIGIT | crTIGIT_12 | 313 | CTTG | CTCCAGCAGGAATACCTGAGC |
| TIGIT | CrTIGIT_13 | 314 | CTTG | GAGCCATGGCCGCGACGCTGG |
| TIGIT | CrTIGIT_14 | 315 | TTTC | TAGTCAACGCGACCACCACGA |
| TIGIT | CrTIGIT_15 | 316 | CTTT | CTAGTCAACGCGACCACCACG |
| TIGIT | CrTIGIT_16 | 317 | TTTG | TAGTTTGTTTGTTTTTAGAAG |
| TIGIT | CrTIGIT_17 | 318 | TTTG | TTTGTTTTTAGAAGAAAGCCC |
| TIGIT | CrTIGIT_18 | 319 | TTTG | TTTTTAGAAGAAAGCCCTCAG |
| TIGIT | CrTIGIT_19 | 320 | TTTT | TAGAAGAAAGCCCTCAGAATC |
| TIGIT | CrTIGIT_20 | 321 | CTTC | CACAGAATGGATTCTGAGGGC |
| TIGIT | CrTIGIT_21 | 322 | TTTT | CTCCTGAGGTCACCTTCCACA |
| TIGIT | CrTIGIT_22 | 323 | CTTC | CTGGGGGTGAGGGAGCACTGG |
| TIGIT | CrTIGIT_23 | 324 | CTTC | TGCCTGGACACAGCTTCCTGG |
| TIGIT | CrTIGIT_24 | 325 | CTTC | GTCCTCTTCCCTAGGAATGAT |
| TIGIT | CrTIGIT_25 | 326 | CTTC | TGTAACTCAGGACATTGAAGT |
| TIGIT | CrTIGIT_26 | 327 | CTTC | AATGTCCTGAGTTACAGAAGC |
| TIGIT | CrTIGIT_27 | 328 | TTTC | TATTGTGCCTGTCATCATTCC |
| TIGIT | CrTIGIT_28 | 329 | TTTC | TCTGCAGAAATGTTCCCCGTT |
| TIGIT | CrTIGIT_29 | 330 | CTTT | CTCTGCAGAAATGTTCCCCGT |
| TIGIT | CrTIGIT_30 | 331 | CTTG | TGCCGTGGTGGAGGAGAGGTG |
| TIGIT | crTIGIT_31 | 332 | CTTC | TGGCCATTTGTAATGCTGACT |
| TIM3 | crTIM3_1 | 333 | CTTA | CTTGTAAGTAGTAGCAGCAGC |
| TIM3 | crTIM3_2 | 334 | TTTC | CAAGGATGCTTACCACCAGGG |
| TIM3 | crTIM3_3 | 335 | CTTG | TAAGTAGTAGCAGCAGCAGCA |
| TIM3 | crTIM3_4 | 336 | CTTA | CCACCAGGGGACATGGCCCAG |
| TIM3 | crTIM3_5 | 337 | TTTG | AATGTGGCAACGTGGTGCTCA |
| TIM3 | crTIM3_6 | 338 | CTTT | TCTTCTGCAAGCTCCATGTTT |
| TIM3 | crTIM3_7 | 339 | CTTT | GCCCCAGCAGACGGGCACGAG |
| TIM3 | crTIM3_8 | 340 | TTTC | ATCAGTCCTGAGCACCACGTT |
| TIM3 | crTIM3_9 | 341 | CTTT | CATCAGTCCTGAGCACCACGT |
| TIM3 | crTIM3_10 | 342 | TTTA | GCCAGTATCTGGATGTCCAAT |
| TIM3 | crTIM3_11 | 343 | TTTG | CGGAAATCCCCATTTAGCCAG |
| TIM3 | crTIM3_12 | 344 | CTTT | GCGGAAATCCCCATTTAGCCA |
| TIM3 | crTIM3_13 | 345 | TTTC | CGCAAAGGAGATGTGTCCCTG |
| TIM3 | crTIM3_14 | 346 | TTTG | GATCCGGCAGCAGTAGATCCC |
| TIM3 | crTIM3_15 | 347 | TTTT | TCATCATTCATTATGCCTGGG |
| TIM3 | crTIM3_16 | 348 | TTTT | CTTCTGCAAGCTCCATGTTTT |
| TIM3 | crTIM3_17 | 349 | CTTC | AGGTTAAATTTTTCATCATTC |
| TIM3 | crTIM3_18 | 350 | TTTG | ATGACCAACTTCAGGTTAAAT |
| TIM3 | crTIM3_19 | 351 | TTTA | ACCTGAAGTTGGTCATCAAAC |
| TIM3 | crTIM3_20 | 352 | CTTA | TGTTGTTTCTGACATTAGCCA |
| TIM3 | crTIM3_21 | 353 | TTTC | TGACATTAGCCAAGGTCACCC |
| TIM3 | crTIM3_22 | 354 | CTTG | GAAAGGCTGCAGTGAAGTCTC |
| TIM3 | crTIM3_23 | 355 | CTTC | ACTGCAGCCTTTCCAAGGATG |
| TIM3 | crTIM3_24 | 356 | CTTT | CCAAGGATGCTTACCACCAGG |
| TIM3 | crTIM3_25 | 357 | TTTT | CACATCTTCCCTTTGACTGTG |
| TIM3 | crTIM3_26 | 358 | TTTT | TATAGCAGAGACACAGACACT |
| TIM3 | crTIM3_27 | 359 | TTTA | TATCAGGGAGGCTCCCCAGTG |
| TIM3 | CrTIM3_28 | 360 | CTTA | CTGTTAGATTTATATCAGGGA |
| TIM3 | CrTIM3_29 | 361 | TTTG | TGTTTCCATAGCAAATATCCA |
| TIM3 | crTIM3_30 | 362 | TTTC | CATAGCAAATATCCACATTGG |
| TIM3 | crTIM3_31 | 363 | CTTA | CGGGACTCTGGAGCAACCATC |
| TIM3 | crTIM3_32 | 364 | TTTG | AAAATTAAAGCGCCGAAGATA |
| TIM3 | crTIM3_33 | 365 | CTTA | CATTTGAAAATTAAAGCGCCG |
| TIM3 | crTIM3_34 | 366 | CTTT | TGTTTCCCCCTTACTAGGGTA |
| TIM3 | crTIM3_35 | 367 | TTTT | GTTTCCCCCTTACTAGGGTAT |
| TIM3 | CrTIM3_36 | 368 | CTTT | GACTGTGTCCTGCTGCTGCTG |
| TIM3 | crTIM3_37 | 369 | TTTC | CCCCTTACTAGGGTATTCTCA |
| TIM3 | crTIM3_38 | 370 | CTTA | CTAGGGTATTCTCATAGCAAA |
| TIM3 | crTIM3_39 | 371 | CTTA | AATTCTGTATCTTCTCTTTGC |
| TIM3 | crTIM3_40 | 372 | CTTT | ATTTCCACAGCCTCATCTCTT |
| TIM3 | crTIM3_41 | 373 | TTTA | TTTCCACAGCCTCATCTCTTT |
| TIM3 | crTIM3_42 | 374 | TTTC | CACAGCCTCATCTCTTTGGCC |
| TIM3 | crTIM3_43 | 375 | TTTG | GCCAACCTCCCTCCCTCAGGA |
| TIM3 | crTIM3_44 | 376 | TTTG | CCAATCCTGAGGGAGGGAGGT |
| TIM3 | crTIM3_45 | 377 | TTTT | CTTCTGAGCGAATTCCCTCTG |
| TIM3 | crTIM3_46 | 378 | CTTC | ATATACGTTCTCTTCAATGGT |
| TIM3 | crTIM3_47 | 379 | CTTT | GGGTTGTCGCTTTGCAATGCC |
| TIM3 | crTIM3_48 | 380 | TTTG | GGTTGTCGCTTTGCAATGCCA |
| TIM3 | crTIM3_49 | 381 | CTTC | TCTCTCTATGCAGGGTCCTCA |
| TIM3 | crTIM3_50 | 382 | CTTC | TACACCCCAGCCGCCCCAGGG |
| TIM3 | crTIM3_51 | 383 | TTTG | CCCCAGCAGACGGGCACGAGG |
| AAVS1 | crAAVS1 | 384 | TTTC | TTAGGATGGCCTTCTCCGACG |

TABLE 6
Spacer sequences

| Target | | SEQ ID | |
|---|---|---|---|
| Gene | Name | NO | Spacer Sequence |
| APLNR | RNA0235_gAPLNR_002 | 1549 | CAGTCTGTGTACTCACACTCA |
| APLNR | RNA0236_gAPLNR_001 | 1550 | ACAACTACTATGGGGCAGACA |
| APLNR | RNA0237_gAPLNR_008 | 1551 | CCCTGTGCTGGATGCCCTACC |
| APLNR | RNA0238_gAPLNR_011 | 1552 | TCGTGCATCTGTTCTCCACCC |
| APLNR | RNA0239_gAPLNR_003 | 1553 | GGAGCAGCCGGGAGAAGAGGC |
| APLNR | RNA0240_gAPLNR_010 | 1554 | GACCCCCGCTTCCGCCAGGCC |
| APLNR | RNA0241_gAPLNR_007 | 1555 | GGCGATGAAGAAGTAACAGGT |
| APLNR | RNA0242_gAPLNR_009 | 1556 | ACCTCTTCCTCATGAACATCT |
| APLNR | RNA0243_gAPLNR_004 | 1557 | GGACCTTCTTCTGCAAGCTCA |
| APLNR | RNA0244_gAPLNR_006 | 1558 | TGGTGCCCTTCACCATCATGC |
| BBS1 | RNA0245_gBBS1_005 | 1559 | CATGGGGATGGGGAATACAAG |
| BBS1 | RNA0246_gBBS1_015 | 1560 | ACTTAGCTCCAGCTGCAGAAA |
| BBS1 | RNA0247_gBBS1_007 | 1561 | GGTCATCACCAGTGGTCCITT |
| BBS1 | RNA0248_gBBS1_032 | 1562 | CGTGGATCAGACACTGCGAGA |
| BBS1 | RNA0249_gBBS1_016 | 1563 | CAAATGCCTCCATTTCACTTA |
| BBS1 | RNA0250_gBBS1_018 | 1564 | TAAACCAACACAAGTCCAACT |
| BBS1 | RNA0251_gBBS1_009 | 1565 | GCCTGGTTCCAAAGGTCTTGT |
| BBS1 | RNA0252_gBBS1_033 | 1566 | TCCACCCACCCTCTCCATAGG |
| BBS1 | RNA0253_gBBS1_028 | 1567 | CACTGTCCACTTCCCTAGGTG |
| BBS1 | RNA0254_gBBS1_017 | 1568 | TGCAGCTGGAGCTAAGTGAAA |
| CALR | RNA0225_gCALR_019 | 1539 | TGGGTGGATCCAAGTGCCCTT |
| CALR | RNA0226_gCALR_013 | 1540 | GACCAGACAGACATGCACGGA |
| CALR | RNA0227_gCALR_015 | 1541 | CACACCTGTACACACTGATTG |
| CALR | RNA0228_gCALR_012 | 1542 | CTAATAGTTTGGACCAGACAG |
| CALR | RNA0229_gCALR_001 | 1543 | GATTCGATCCAGCGGGAAGTC |
| CALR | RNA0230_gCALR_021 | 1544 | CTCCAAGTCTCACCTGCCAGA |
| CALR | RNA0231_gCALR_006 | 1545 | CAGACAAGCCAGGATGCACGC |
| CALR | RNA0232_gCALR_011 | 1546 | ACCGTGAACTGCACCACCAGC |
| CALR | RNA0233_gCALR_014 | 1547 | CCACCACCCCCAGGCACACCT |
| CALR | RNA0234_gCALR_017 | 1548 | AAGCATCAGGATCCTTTATCT |
| CD247 | RNA0210_gCD247_002 | 86 | ACCGCGGCCATCCTGCAGGCA |
| CD247 | RNA0207_gCD247_001 | 87 | TGAGGGAAAGGACAAGATGAA |
| CD247 | RNA0206_gCD247_004 | 88 | GGATCCAGCAGGCCAAAGCTC |
| CD247 | RNA0208_gCD247_011 | 89 | CTAGCAGAGAAGGAAGAACCC |
| CD247 | RNA0214_gCD247_007 | 90 | TGTGTTGCAGTTCAGCAGGAG |
| CD247 | RNA0213_gCD247_012 | 95 | ATCCCAATCTCACTGTAGGCC |
| CD247 | RNA0205_gCD247_013 | 99 | ACTCCCAAACAACCAGCGCCG |
| CD247 | RNA0212_gCD247_015 | 103 | CTTTCACGCCAGGGTCTCAGT |
| CD247 | RNA0211_gCD247_016 | 104 | ACGCCAGGGTCTCAGTACAGC |
| CD247 | RNA0209_gCD247_005 | 1528 | GCCTGCTGGATCCCAAACTCT |
| CD38 | RNA0415_gCD38_001 | 1729 | TCCCCGGACACCGGGCTGAAC |
| CD38 | RNA0416_gCD38_002 | 1730 | AGTGTACTTGACGCATCGCGC |
| CD38 | RNA0417_gCD38_003 | 1731 | CCGAGACCGTCCTGGCGCGAT |
| CD38 | RNA0418_gCD38_004 | 1732 | GCAGTCTACATGTCTGAGATA |
| CD38 | RNA0419_gCD38_005 | 1733 | TGTGTTTTATCTCAGACATGT |
| CD38 | RNA0420_gCD38_006 | 1734 | TCTCAGACATGTAGACTGCCA |
| CD38 | RNA0421_gCD38_007 | 1735 | AAATAAATGCACCCTTGAAAG |
| CD38 | RNA0422_gCD38_008 | 1736 | AAGGGTGCATTTATTTCAAAA |
| CD38 | RNA0423_gCD38_009 | 1737 | TTTCAAAACATCCTTGCAACA |
| CD38 | RNA0424_gCD38_010 | 1738 | AAAACATCCTTGCAACATTAC |
| CD38 | RNA0425_gCD38_011 | 1739 | TTCTGCTCCAAAGAAGAATCT |
| CD38 | RNA0426_gCD38_012 | 1740 | TTCTTCCTTAGATTCTTCTTT |
| CD38 | RNA0427_gCD38_013 | 1741 | GAGCAGAATAAAAGATCTGGC |
| CD38 | RNA0428_gCD38_014 | 1742 | TACAAACTATGTCTTTTAGAA |
| CD38 | RNA0429_gCD38_015 | 1743 | TCCAGTCTGGGCAAGATTGAT |
| CD38 | RNA0430_gCD38_016 | 1744 | GAAATAAACTATCAATCTTGC |
| CD38 | RNA0431_gCD38_017 | 1745 | CAGAATACTGAAACAGGGTTG |
| CD38 | RNA0432_gCD38_018 | 1746 | AGTATTCTGGAAAACGGTTTC |
| CD38 | RNA0433_gCD38_019 | 1747 | ACTACTTGGTACTTACCCTGC |
| CD38 | RNA0434_gCD38_020 | 1748 | AGTTTGCAGAAGCTGCCTGTG |
| CD38 | RNA0435_gCD38_021 | 1749 | CAGAAGCTGCCTGTGATGTGG |
| CD38 | RNA0436_gCD38_022 | 1750 | CTGCGGGATCCATTGAGCATC |
| CD38 | RNA0437_gCD38_023 | 1751 | TCAAAGATTTTACTGCGGGAT |
| CD38 | RNA0438_gCD38_024 | 1752 | GGGTTCTTTGTTTCTTCTATT |
| CD38 | RNA0439_gCD38_025 | 1753 | TTTCTTCTATTTTAGCACTTT |
| CD38 | RNA0440_gCD38_026 | 1754 | TTCTATTTTAGCACTTTTGGG |
| CD38 | RNA0441_gCD38_027 | 1755 | GCACTTTTGGGAGTGTGGAAG |
| CD38 | RNA0442_gCD38_028 | 1756 | GGAGTGTGGAAGTCCATAATT |
| CD38 | RNA0443_gCD38_029 | 1757 | CAACCAGAGAAGGTTCAGACA |
| CD38 | RNA0444_gCD38_030 | 1758 | TGGTGGGATCCTGGCATAAGT |
| CD38 | RNA0445_gCD38_031 | 1759 | TTCCCCAGAGACTTATGCCAG |
| CD38 | RNA0446_gCD38_032 | 1760 | CTTATAATCGATTCCAGCTCT |
| CD38 | RNA0447_gCD38_033 | 1761 | CTTTTTTGCTTTCTTGTCATA |
| CD38 | RNA0448_gCD38_034 | 1762 | CTTTCTTGTCATAGACCTGAC |
| CD38 | RNA0449_gCD38_035 | 1763 | ACACACTGAAGAAACTTGTCA |
| CD38 | RNA0450_gCD38_036 | 1764 | TTGTCATAGACCTGACAAGTT |
| CD38 | RNA0451_gCD38_037 | 1765 | TTCAGTGTGTGAAAAATCCTG |
| CD3G | RNA0195_gCD3G_017 | 1518 | CCTCTCGACTGGCGAACTCCA |
| CD3G | RNA0196_gCD3G_004 | 1519 | GCTTCTGCATCACAAGTCAGA |
| CD3G | RNA0197_gCD3G_011 | 1520 | GTTCAATGCAGTTCTGACACA |
| CD3G | RNA0198_gCD3G_001 | 1521 | CCGGAGGACAGAGACTGACAT |
| CD3G | RNA0199_gCD3G_012 | 1522 | CCTACAGTGTGTCAGAACTGC |
| CD3G | RNA0200_gCD3G_007 | 1523 | AAGATGGGAAGATGATCGGCT |
| CD3G | RNA0201_gCD3G_022 | 1524 | CTTGAAGGTGGCTGTACTGGT |
| CD3G | RNA0202_gCD3G_008 | 1525 | CACTGATACATCCCTCGAGGG |
| CD3G | RNA0203_gCD3G_006 | 1526 | TCTTCAGTTAGGAAGCCGATC |
| CD3G | RNA0204_gCD3G_023 | 1527 | CAGGTACTTTGGCCCAGTCAA |
| CD52 | RNA0143_gCD52_1 | 985 | CTCTTCCTCCTACTCACCATC |
| CD52 | RNA0144_gCD52_4 | 1039 | GCTGGTGTCGTTTTGTCCTGA |
| CD52 | RNA0141_gCD52_9 | 1466 | TTCGTGGCCAATGCCATAATC |
| CD52 | RNA0142_gCD52_10 | 1467 | TCCTGAGAGTCCAGTTTGTAT |
| CD58 | RNA0255_gCD58_033 | 1569 | GGTATTCTGAAATGTGACAGA |
| CD58 | RNA0256_gCD58_005 | 1570 | AAGGCACATTGCTTGGTACAT |
| CD58 | RNA0257_gCD58_004 | 1571 | CCAACAAATATATGGTGTTGT |
| CD58 | RNA0258_gCD58_020 | 1572 | CATTGCTCCATAGGACAATCC |
| CD58 | RNA0259_gCD58_023 | 1573 | AGATGGAAAATGATCTTCCAC |
| CD58 | RNA0260_gCD58_012 | 1574 | AAAGATGAGAAAGCTCTGAAT |
| CD58 | RNA0261_gCD58_028 | 1575 | TAGGTCATTCAAGACACAGAT |
| CD58 | RNA0262_gCD58_019 | 1576 | CAGAGTCTCTTCCATCTCCCA |
| CD58 | RNA0263_gCD58_010 | 1577 | AAAGAGGTCCTATGGAAAAAA |
| CD58 | RNA0264_gCD58_018 | 1578 | GCGATTCCATTTCATACTCAT |
| COL17A1 | RNA0265_gCOL17A1_084 | 1579 | AGAGGGGTCATCGATGCTCAC |
| COL17A1 | RNA0266_gCOL17A1_070 | 1580 | GGTGACAAAGGACCAATGGGA |
| COL17A1 | RNA0267_gCOL17A1_006 | 1581 | GCATAGCCATTGCTGGTCCCG |
| COL17A1 | RNA0268_gCOL17A1_024 | 1582 | CAGTGTCAGGCACCTACGATG |
| COL17A1 | RNA0269_gCOL17A1_094 | 1583 | ATGCCGGCTCTACTGTACCTT |
| COL17A1 | RNA0270_gCOL17A1_054 | 1584 | AGGTGACATGGGAAGTCCAGG |
| COL17A1 | RNA0271_gCOL17A1_005 | 1585 | TAGTTGTCACTGAAACAGTAA |
| COL17A1 | RNA0272_gCOL17A1_065 | 1586 | CAAGAAGCAGCAAACTGACCT |
| COL17A1 | RNA0273_gCOL17A1_047 | 1587 | CTGTTCCATCATTAGCTTCTT |
| COL17A1 | RNA0274_gCOL17A1_017 | 1588 | ACTCCGTCCTCTGGTTGAAGA |
| CSF1R | RNA0478_gCSF1R_001 | 1792 | CAGAGAGTGCCTACTTGAACT |
| CSF1R | RNA0479_gCSF1R_002 | 1793 | TGGTCCCTCCCACCCTCAGGA |
| DEFB134 | RNA0275_gDEFB134_007 | 1589 | CTTCCAGGTATAAATTCATTA |
| DEFB134 | RNA0276_gDEFB134_001 | 1590 | CCTGCCAGCACTGGATCCCAA |
| DEFB134 | RNA0277_gDEFB134_012 | 1591 | CTTTGACACAGCACTCCAGCT |
| DEFB134 | RNA0278_gDEFB134_010 | 1592 | ACTCTCATAGCATTCAAGTCT |
| DEFB134 | RNA0279_gDEFB134_004 | 1593 | CTTTGGGATCCAGTGCTGGCA |
| DEFB134 | RNA0280_gDEFB134_013 | 1594 | AGCTGGAGTGCTGTGTCAAAG |
| DEFB134 | RNA0281_gDEFB134_014 | 1595 | TTATGTCAGGGTGCAGGATTT |
| DEFB134 | RNA0282_gDEFB134_011 | 1596 | ACACAGCACTCCAGCTGAAAC |
| DEFB134 | RNA0283_gDEFB134_009 | 1597 | TAGCATTTCTTGTGCATTTCT |
| DEFB134 | RNA0284_gDEFB134_008 | 1598 | TTGTGCATTTCTGATGATAAT |
| ERAP1 | RNA0345_gERAP1_037 | 1659 | AGCATACCGTATCCCCTACCC |
| ERAP1 | RNA0346_gERAP1_077 | 1660 | CCCTAATAACCATCACAGTGA |
| ERAP1 | RNA0347_gERAP1_035 | 1661 | GGTAGGGGATACGGTATGCTG |
| ERAP1 | RNA0348_gERAP1_078 | 1662 | CTCTAGGAGCATTACCCAGTG |
| ERAP1 | RNA0349_gERAP1_029 | 1663 | AGTCTGTCAGCAAGATAACCA |
| ERAP1 | RNA0350_gERAP1_008 | 1664 | CATGGATCAAGAGATCATAAT |
| ERAP1 | RNA0351_gERAP1_065 | 1665 | AATGCGTCAGCACTAAGATAC |
| ERAP1 | RNA0352_gERAP1_061 | 1666 | CCTTATCATAAGAAACATCAT |
| ERAP1 | RNA0353_gERAP1_039 | 1667 | CATAGCACCAGACTGAAAGTC |
| ERAP1 | RNA0354_gERAP1_015 | 1668 | CAAAAGCACCTACAGAACCAA |
| ERAP2 | RNA0355_gERAP2_046 | 1669 | GAGAGTGGATAGTAGATATCA |
| ERAP2 | RNA0356_gERAP2_018 | 1670 | AGTTACCCTGCTCATGAACAA |
| ERAP2 | RNA0357_gERAP2_099 | 1671 | ATGTGGACTCAAATGGTTACT |
| ERAP2 | RNA0358_gERAP2_118 | 1672 | GAGCAATATGAACTGTCAATG |
| ERAP2 | RNA0359_gERAP2_001 | 1673 | TGTGTGAATTAACCATTGCAG |
| ERAP2 | RNA0360_gERAP2_134 | 1674 | ACTTGGGCTCATATGACATAA |
| ERAP2 | RNA0361_gERAP2_048 | 1675 | ATATCTACTATCCACTCTCCA |
| ERAP2 | RNA0362_gERAP2_261 | 1676 | TCCTTACCATGTTACTTGTCA |
| ERAP2 | RNA0363_gERAP2_108 | 1677 | CCTGTCAATCACTGGCTTAAA |
| ERAP2 | RNA0364_gERAP2_014 | 1678 | ATGTATCTTGAATCTTCCTCT |
| FAS | RNA0467_gFAS_93 | 1781 | TATTTTTCAGATGTTGACTTG |
| FAS | RNA0468_gFAS_94 | 1782 | AGATGTTGACTTGAGTAAATA |
| FAS | RNA0469_gFAS_95 | 1783 | ACTTGACTTAGTGTCATGACT |
| FAS | RNA0470_gFAS_96 | 1784 | GCTTCATTGACACCATTCTTT |
| FAS | RNA0471_gFAS_97 | 1785 | AGATCTTTAATCAATGTGTCA |
| FAS | RNA0472_gFAS_98 | 1786 | TCTGCAAGAGTACAAAGATTG |
| FAS | RNA0473_gFAS_99 | 1787 | TGAGTCACTAGTAATGTCCTT |
| FAS | RNA0474_gFAS_100 | 1788 | CTTTCTAGGAAACAGTGGCAA |
| FAS | RNA0475_gFAS_101 | 1789 | CTTTCTGTGCTTTCTGCATGT |
| FAS | RNA0476_gFAS_102 | 1790 | CCAATTCCACTAATTGTTTGG |
| FAS | RNA0477_gFAS_103 | 1791 | TAGATGTGAACATGGAATCAT |
| mir-101-2 | RNA0305_gmir-101- | 1619 | GGTTATCATGGTACCGATGCT |
| | 2_001 | | |
| mir-101-2 | RNA0306_gmir-101- | 1620 | AGATATACAGCATCGGTACCA |
| | 2_002 | | |
| mir-101-2 | RNA0307_gmir-101- | 1621 | TCAATGTGATGGCACCACCAT |
| | 2_003 | | |
| IFNGR1 | RNA0365_gIFNGR1_025 | 1679 | AGTTGTAACACCCCACACATG |
| IFNGR1 | RNA0366_gIFNGR1_006 | 1680 | CCGTAGAGGTAAAGAACTATG |
| IFNGR1 | RNA0367_gIFNGR1_042 | 1681 | GAGACAAAACCTGAATCAAAA |
| IFNGR1 | RNA0368_gIFNGR1_008 | 1682 | GTGTTAAGAATTCAGAATGGA |
| IFNGR1 | RNA0369_gIFNGR1_010 | 1683 | ATGGATCACCAACATGATCAG |
| IFNGR1 | RNA0370_gIFNGR1_004 | 1684 | TTACAGTGCCTACACCAACTA |
| IFNGR1 | RNA0371_gIFNGR1_049 | 1685 | AGTAGTAACCAGTCTGAACCT |
| IFNGR1 | RNA0372_gIFNGR1_012 | 1686 | ACTCTGACCCAAAGAGAATTT |
| IFNGR1 | RNA0373_gIFNGR1_021 | 1687 | GGGATCATAATCGACTTCCTG |
| IFNGR1 | RNA0374_gIFNGR1_052 | 1688 | TGGAGTGATCACTCTCAGAAC |
| IFNGR2 | RNA0375_gIFNGR2_012 | 1689 | CCAGTAATGGACATAATAACA |
| IFNGR2 | RNA0376_gIFNGR2_021 | 1690 | GTAGCAAGATATGTTGCTTAA |
| IFNGR2 | RNA0377_gIFNGR2_001 | 1691 | TCTGTCCCCCTCAAGACCCTC |
| IFNGR2 | RNA0378_gIFNGR2_006 | 1692 | AATGTCACTCTACGCCTTCGA |
| IFNGR2 | RNA0379_gIFNGR2_017 | 1693 | ATTGGATAACTTAAAACCCTC |
| IFNGR2 | RNA0380_gIFNGR2_005 | 1694 | CTTCCCAGCACCGACAGTAAA |
| IFNGR2 | RNA0381_gIFNGR2_015 | 1695 | AGTTATCCAATGAAATGGAGT |
| IFNGR2 | RNA0382_gIFNGR2_031 | 1696 | ACACTCCACCAAGCATCCCAT |
| IFNGR2 | RNA0383_gIFNGR2_026 | 1697 | GCCTCCACTGAGCTTCAGCAA |
| IFNGR2 | RNA0384_gIFNGR2_003 | 1698 | AACTGCACTTGGTAGACAACA |
| JAK1 | RNA0385_gJAK1_021 | 1699 | GCTACAAGCGATATATTCCAG |
| JAK1 | RNA0386_gJAK1_090 | 1700 | AGATCAGCTATGTGGTTACCT |
| JAK1 | RNA0387_gJAK1_100 | 1701 | CCTTACAAATCTGAACGGCAT |
| JAK1 | RNA0388_gJAK1_108 | 1702 | ACCAAAGCAATTGAAACCGAT |
| JAK1 | RNA0389_gJAK1_075 | 1703 | CCAGAGCGTGGTTCCAAAGCT |
| JAK1 | RNA0390_gJAK1_002 | 1704 | CTTCCACAACAGTATCTAAAT |
| JAK1 | RNA0391_gJAK1_074 | 1705 | GTACACACATTTCCATGGACC |
| JAK1 | RNA0392_gJAK1_059 | 1706 | GCATGAAGCTGATGTTATCCG |
| JAK1 | RNA0393_gJAK1_037 | 1707 | ATTCGAATGACGGTGGAAACG |
| JAK1 | RNA0394_gJAK1_111 | 1708 | GATTGCATTAAACATTCTGGA |
| JAK2 | RNA0395_gJAK2_187 | 1709 | GGTTAACCAAAGTCTTGCCAC |
| JAK2 | RNA0396_gJAK2_118 | 1710 | AGATATGTATCTAGTGATCCA |
| JAK2 | RNA0397_gJAK2_137 | 1711 | CCACAAAGTGGTACCAAAACT |
| JAK2 | RNA0398_gJAK2_009 | 1712 | GAAGCAGCAATACAGATTTCT |
| JAK2 | RNA0399_gJAK2_132 | 1713 | AATGCATTCAGGTGGTACCCA |
| JAK2 | RNA0400_gJAK2_191 | 1714 | CAGGTATGCTCCAGAATCACT |
| JAK2 | RNA0401_gJAK2_175 | 1715 | AAGATAGTCTCGTAAACTTCC |
| JAK2 | RNA0402_gJAK2_101 | 1716 | AAGGCGTACGAAGAGAAGTAG |
| JAK2 | RNA0403_gJAK2_121 | 1717 | GATCACTAGATACATATCTGA |
| JAK2 | RNA0404_gJAK2_126 | 1718 | GCACATACATTCCCATGAATA |
| MLANA | RNA0295_gMLANA_003 | 1609 | GTCTTCTACAATACCAACAGC |
| MLANA | RNA0296_gMLANA_020 | 1610 | TCATAAGCAGGTGGAGCATTG |
| MLANA | RNA0297_gMLANA_010 | 1611 | CTGTCCCGATGATCAAACCCT |
| MLANA | RNA0298_gMLANA_004 | 1612 | CCAACCATCAAGGCTCTGTAT |
| MLANA | RNA0299_gMLANA_011 | 1613 | TCTTGAAGAGACACTTTGCTG |
| MLANA | RNA0300_gMLANA_009 | 1614 | AGGATAAAAGTCTTCATGTTG |
| MLANA | RNA0301_gMLANA_001 | 1615 | AACTTACTCTTCAGCCGTGGT |
| MLANA | RNA0302_gMLANA_008 | 1616 | CATTTCAGGATAAAAGTCTTC |
| MLANA | RNA0303_gMLANA_002 | 1617 | TCTATCTCTTGGGCCAGGGCC |
| MLANA | RNA0304_gMLANA_012 | 1618 | ATCATCGGGACAGCAAAGTGT |
| PSMB5 | RNA0215_gPSMB5_007 | 1529 | GAGGCAGCTGCTACAGAGATG |
| PSMB5 | RNA0216_gPSMB5_005 | 1530 | CTCTGATCTTAACAGTTCCGC |
| PSMB5 | RNA0217_gPSMB5_006 | 1531 | GAAGCTCATAGATTCGACATT |
| PSMB5 | RNA0218_gPSMB5_011 | 1532 | AGGGGCCACCTTCTCTGTAGG |
| PSMB5 | RNA0219_gPSMB5_012 | 1533 | AGGGGGTAGAGCCACTATACT |
| PSMB5 | RNA0220_gPSMB5_010 | 1534 | CAGGCCTCTACTACGTGGACA |
| PSMB5 | RNA0221_gPSMB5_002 | 1535 | GGACTTGGGGGTCGTGCAGAT |
| PSMB5 | RNA0222_gPSMB5_001 | 1536 | TGCCCACACTAGACATGGCGC |
| PSMB5 | RNA0223_gPSMB5_003 | 1537 | GATTCCTGGCTCTTCTGGGAC |
| PSMB5 | RNA0224_gPSMB5_008 | 1538 | TACTGATACACCATGTTGGCA |
| PSMB8 | RNA0155_gPSMB8_011 | 1478 | CTGAGAGCCGAGTCCCATGTT |
| PSMB8 | RNA0156_gPSMB8_001 | 1479 | TCTATGCGATCTCCAGAGCTC |
| PSMB8 | RNA0157_gPSMB8_004 | 1480 | TCTTATCAGCCCACAGAATTC |
| PSMB8 | RNA0158_gPSMB8_014 | 1481 | TCCACAGTGTACCACATGAAG |
| PSMB8 | RNA0159_gPSMB8_010 | 1482 | ATCTTATAGGGTCCTGGACTC |
| PSMB8 | RNA0160_gPSMB8_013 | 1483 | ACCCAACCATCTTCCTTCATG |
| PSMB8 | RNA0161_gPSMB8_008 | 1484 | AGTGTCGGCAGCCTCCAAGCT |
| PSMB8 | RNA0162_gPSMB8_015 | 1485 | TACTTTCACCCAACCATCTTC |
| PSMB8 | RNA0163_gPSMB8_012 | 1486 | TCATTTGTCCACAGTGTACCA |
| PSMB8 | RNA0164_gPSMB8_005 | 1487 | TCCGTCCCCACCCAGGGACTG |
| PSMB9 | RNA0145_gPSMB9_010 | 1468 | GGAGAAACTCACCTGACCTCC |
| PSMB9 | RNA0146_gPSMB9_011 | 1469 | ACCTGAGGATCCCTTTCCCAG |
| PSMB9 | RNA0147_gPSMB9_005 | 1470 | CCTCAGGATAGAACTGGAGGA |
| PSMB9 | RNA0148_gPSMB9_009 | 1471 | GCTGCTGCAAATGTGGTGAGA |
| PSMB9 | RNA0149_gPSMB9_012 | 1472 | CCAGGTATATGGAACCCTGGG |
| PSMB9 | RNA0150_gPSMB9_015 | 1473 | GCAGTTCATTGCCCAAGATGA |
| PSMB9 | RNA0151_gPSMB9_007 | 1474 | TCACCACATTTGCAGCAGCCA |
| PSMB9 | RNA0152_gPSMB9_001 | 1475 | ACGGGGGCGTTGTGATGGGTT |
| PSMB9 | RNA0153_gPSMB9_014 | 1476 | TCTATGGTTATGTGGATGCAG |
| PSMB9 | RNA0154_gPSMB9_002 | 1477 | CTCACCCTGCAGACACTCGGG |
| PTCD2 | RNA0324_gPTCD2_018 | 1638 | ATTACCAGGTACCATGCAGAG |
| PTCD2 | RNA0325_gPTCD2_043 | 1639 | GCTGTGGCATTAGCTCTGAAT |
| PTCD2 | RNA0326_gPTCD2_042 | 1640 | CCTGATTCAGAGCTAATGCCA |
| PTCD2 | RNA0327_gPTCD2_011 | 1641 | GTGCCAGAAAGATTACATGCA |
| PTCD2 | RNA0328_gPTCD2_033 | 1642 | GCAGGTGCTTTGCAAGTATTG |
| PTCD2 | RNA0329_gPTCD2_064 | 1643 | ATAGCAACGTGTGAGATTTCC |
| PTCD2 | RNA0330_gPTCD2_032 | 1644 | ATCTCTATCAATACTTGCAAA |
| PTCD2 | RNA0331_gPTCD2_007 | 1645 | GCTAAAAGATACCTACTTACA |
| PTCD2 | RNA0332_gPTCD2_026 | 1646 | TTCTCAGACTCCACATCATTC |
| PTCD2 | RNA0333_gPTCD2_005 | 1647 | ACCACATTATCTGTAAGTAGG |
| RFX5 | RNA0165_gRFX5_028 | 1488 | GCATCACTTGCTGTATCCTCT |
| RFX5 | RNA0166_gRFX5_015 | 1489 | GTACTTACACTCTCAGAACCC |
| RFX5 | RNA0167_gRFX5_018 | 1490 | GATGACCGTTCCCGAGGTGCA |
| RFX5 | RNA0168_gRFX5_008 | 1491 | TGTAGCTCAGAGCCAAGTACA |
| RFX5 | RNA0169_gRFX5_017 | 1492 | GTACCTCTGCAGAAGAGGACG |
| RFX5 | RNA0170_gRFX5_016 | 1493 | AGGATCCGCTCTGCCCAGTCA |
| RFX5 | RNA0171_gRFX5_026 | 1494 | GCTGGTGGAGCCTGCCCACTG |
| RFX5 | RNA0172_gRFX5_013 | 1495 | ACTTGCATCAGATATTGCTAC |
| RFX5 | RNA0173_gRFX5_012 | 1496 | GCAAGATCATCAGAGAGATCT |
| RFX5 | RNA0174_gRFX5_038 | 1497 | GCTTCTGCTGCCCTTGATGAC |
| RFXAP | RNA0175_gRFXAP_016 | 1498 | GAACAAGTGTTAAATCAAAAA |
| REXAP | RNA0176_gRFXAP_012 | 1499 | GGGATCGTCCTGCAAGACCTA |
| REXAP | RNA0177_gRFXAP_025 | 1500 | GAGCAAAGACAACAGCAGTTT |
| RFXAP | RNA0178_gRFXAP_021 | 1501 | TGTAAAAATTGCACTACTTCT |
| REXAP | RNA0179_gRFXAP_023 | 1502 | CAGAAACAGCAACAGCTATTA |
| REXAP | RNA0180_gRFXAP_001 | 1503 | GAGGATCTAGAGGACGAGGAG |
| REXAP | RNA0181_gRFXAP_009 | 1504 | ACAATGGAGAGTATGTTATCT |
| REXAP | RNA0182_gRFXAP_005 | 1505 | CCGCGCTGCCAGTCGAGGCAG |
| REXAP | RNA0183_gRFXAP_020 | 1506 | TAAGTCGTTACTAAGAAGTCC |
| RFXAP | RNA0184_gRFXAP_004 | 1507 | TACTTGTCCTTGTACATCTTG |
| RPL23 | RNA0314_gRPL23_003 | 1628 | GCACCAGAGGACCCACCACGT |
| RPL23 | RNA0315_gRPL23_025 | 1629 | ATGCAGGTTCTGCCATTACAG |
| RPL23 | RNA0316_gRPL23_019 | 1630 | AAGATAATGCAGGAGTCATAG |
| RPL23 | RNA0317_gRPL23_008 | 1631 | TAGGAGCCAAAAACCTGTATA |
| RPL23 | RNA0318_gRPL23_027 | 1632 | CCTTCCCTTTATATCCACAGG |
| RPL23 | RNA0319_gRPL23_021 | 1633 | CTACCTTTCATCTCGCCTTTA |
| RPL23 | RNA0320_gRPL23_026 | 1634 | CAAATATACTGGAGAATCATG |
| RPL23 | RNA0321_gRPL23_014 | 1635 | TTCTCTCAGTACATCCAGCAG |
| RPL23 | RNA0322_gRPL23_013 | 1636 | GTTGTCGAATGACCACTGCTG |
| RPL23 | RNA0323_gRPL23_004 | 1637 | TATCCACAGGACGTGGTGGGT |
| RFXANK | RNA0185_gRFXANK_007 | 1508 | TCCTGCCCCTACCCACGACAG |
| REXANK | RNA0186_gRFXANK_011 | 1509 | CCTGCCCCATCTCAGTGCAAC |
| RFXANK | RNA0187_gRFXANK_005 | 1510 | GAGAGATTGAGACCGTTCGCT |
| RFXANK | RNA0188_gRFXANK_002 | 1511 | CCTGCACCCCTGAGCCTGTGA |
| RFXANK | RNA0189_gRFXANK_001 | 1512 | CCCATGGAGCTTACCCAGCCT |
| REXANK | RNA0190_gRFXANK_008 | 1513 | ACGTGGTTCCCGCGCACAGCG |
| REXANK | RNA0191_gRFXANK_003 | 1514 | CCAGCAGGCAGCTCCCTGAAG |
| REXANK | RNA0192_gRFXANK_010 | 1515 | CGGTATCCCAGGGCCACGGCA |
| REXANK | RNA0193_gRFXANK_006 | 1516 | CCAGGATGTGGGGGTCGGCAC |
| REXANK | RNA0194_gRFXANK_009 | 1517 | CAGCCCGAGGCGCTGACCTCA |
| SOX10 | RNA0308_gSOX10_005 | 1622 | ACTACTCTGACCATCAGCCCT |
| SOX10 | RNA0309_gSOX10_006 | 1623 | GGGCCGGGACAGTGTCGTATA |
| SOX10 | RNA0310_gSOX10_004 | 1624 | GCATCCACACCAGGTGGTGAG |
| SOX10 | RNA0311_gSOX10_001 | 1625 | CTGGCGCCGTTGACGCGCACG |
| SOX10 | RNA0312_gSOX10_003 | 1626 | ATGTGGCTGAGTTGGACCAGT |
| SOX10 | RNA0313_gSOX10_002 | 1627 | TTGTGCTGCATACGGAGCCGC |
| SRP54 | RNA0334_gSRP54_139 | 1648 | AGGATAACTAACCAAGATCTG |
| SRP54 | RNA0335_gSRP54_020 | 1649 | GTGGGTGTCCATGCCTTAACT |
| SRP54 | RNA0336_gSRP54_087 | 1650 | GCACCATCCGTACTGTCTAGT |
| SRP54 | RNA0337_gSRP54_064 | 1651 | ATTGGTACAGGGGAACATATA |
| SRP54 | RNA0338_gSRP54_030 | 1652 | ATATGTGCAGACACATTCAGA |
| SRP54 | RNA0339_gSRP54_096 | 1653 | CCCTCAGGTGGCGACATGTCT |
| SRP54 | RNA0340_gSRP54_011 | 1654 | TCTTAGTTGCTTCACTAGTTT |
| SRP54 | RNA0341_gSRP54_029 | 1655 | TCACCCAGCTAGCATATTATT |
| SRP54 | RNA0342_gSRP54_024 | 1656 | CCACTCCCTTGCAATCCAACA |
| SRP54 | RNA0343_gSRP54_021 | 1657 | GCTTGTAGACCCTGGAGTTAA |
| SRP54 | RNA0344_gSRP54_090 | 1658 | GTAAACAACCAGGAAGAATCC |
| STAT1 | RNA0405_gSTAT1_102 | 1719 | CCTGACATCATTCGCAATTAC |
| STAT1 | RNA0406_gSTAT1_113 | 1720 | GTCACCCTTCTAGACTTCAGA |
| STAT1 | RNA0407_gSTAT1_013 | 1721 | TTCTAACCACTCAAATCTAGG |
| STAT1 | RNA0408_gSTAT1_014 | 1722 | AGGAAGACCCAATCCAGATGT |
| STAT1 | RNA0409_gSTAT1_003 | 1723 | CATGGGAAAACTGTCATCATA |
| STAT1 | RNA0410_gSTAT1_103 | 1724 | GATACAGATACTTCAGGGGAT |
| STAT1 | RNA0411_gSTAT1_005 | 1725 | TAACCACTGTGCCAGGTACTG |
| STAT1 | RNA0412_gSTAT1_026 | 1726 | TAGTGTATAGAGCATGAAATC |
| STAT1 | RNA0413_gSTAT1_032 | 1727 | TGATCACTCTTTGCCACACCA |
| STAT1 | RNA0414_gSTAT1_009 | 1728 | ATGACCTCCTGTCACAGCTGG |
| Tap1 | RNA0111_gTap1_026 | 1436 | GGGAAAAGCTGCAAGAAATAA |
| Tap1 | RNA0112_gTap1_035 | 1437 | GGTAGGCAAAGGAGACATCTT |
| Tap1 | RNA0113_gTap1_039 | 1438 | GAAGAAGTCTTCAAGAAAATA |
| Tap1 | RNA0114_gTap1_033 | 1439 | TCTGAGGAGCCCACAGCCTTC |
| Tap1 | RNA0115_gTap1_016 | 1440 | AGGAGAAACCTGTCTGGTTCT |
| Tap1 | RNA0116_gTap1_011 | 1441 | GAGTGAAGGTATCGGCTGAGC |
| Tap1 | RNA0117_gTap1_036 | 1442 | CCTACCCAAACCGCCCAGATG |
| Tap1 | RNA0118_gTap1_020 | 1443 | CTTCTGCCCAAGAAGGTGGGA |
| Tap1 | RNA0119_gTap1_030 | 1444 | AGGTATGCTGCTGAAAGTGGG |
| Tap1 | RNA0120_gTap1_012 | 1445 | AGCCCCCAGACCTGGCTATGG |
| TAP2 | RNA0121_gTAP2_014 | 1446 | AAGGAAGCCAGTTACTCATCA |
| TAP2 | RNA0122_gTAP2_004 | 1447 | GCAGCCCCCACAGCCCTCCCA |
| TAP2 | RNA0123_gTAP2_027 | 1448 | CAGACCCTGGTATACATATAT |
| TAP2 | RNA0124_gTAP2_028 | 1449 | GCTGTCGGTCCATGTAGGAGA |
| TAP2 | RNA0125_gTAP2_029 | 1450 | TCCTACATGGACCGACAGCCA |
| TAP2 | RNA0126_gTAP2_008 | 1451 | AGGTGAGACATTAATCCCTCA |
| TAP2 | RNA0127_gTAP2_037 | 1452 | ATCCAGCAGCACCTGTCCCCC |
| TAP2 | RNA0128_gTAP2_030 | 1453 | ACAACCCCCTGCAGAGTGGTG |
| TAP2 | RNA0129_gTAP2_038 | 1454 | AGTTGGGCAGGAGCCTGTGCT |
| TAP2 | RNA0130_gTAP2_040 | 1455 | TAGAAGATACCTGTGTATATT |
| TAPBP | RNA0131_gTAPBP_016 | 1456 | CCCACAGCTGTCTACCTGTCC |
| TAPBP | RNA0132_gTAPBP_011 | 1457 | CCCAGAACCCCCCAAAGTGTC |
| TAPBP | RNA0133_gTAPBP_007 | 1458 | AGGAGGGCACCTATCIGGCCA |
| TAPBP | RNA0134_gTAPBP_003 | 1459 | CCTACATGCCCCCCACCTCCG |
| TAPBP | RNA0135_gTAPBP_004 | 1460 | GGCTAGAGTGGCGACGCCAGC |
| TAPBP | RNA0136_gTAPBP_001 | 1461 | CGCTCGCATCCTCCACGAACC |
| TAPBP | RNA0137_gTAPBP_002 | 1462 | GCAGAGGCGGGGAGAGGCACG |
| TAPBP | RNA0138_gTAPBP_013 | 1463 | CTGTCTGCCTTTCTTCTGCTT |
| TAPBP | RNA0139_gTAPBP_010 | 1464 | GTCCTCTTTCCCCAGAACCCC |
| TAPBP | RNA0140_gTAPBP_012 | 1465 | AGGGCCCTCCCTTGAGGACAG |
| TGFBR2 | RNA0452_gTGFBR2_001 | 1766 | GATCTCTTTCCCGCTACAGGG |
| TGFBR2 | RNA0453_gTGFBR2_002 | 1767 | CCGCTACAGGGCATCCAGATG |
| TGFBR2 | RNA0454_gTGFBR2_003 | 1768 | GGGAGCCGTCTTCAGGAATCT |
| TGFBR2 | RNA0455_gTGFBR2_004 | 1769 | GTAGTGTTTAGGGAGCCGTCT |
| TGFBR2 | RNA0456_gTGFBR2_005 | 1770 | CTATAGGTGGGAACTGCAAGA |
| TGFBR2 | RNA0457_gTGFBR2_006 | 1771 | GAGAATGTTGAGTCCTTCAAG |
| TGFBR2 | RNA0458_gTGFBR2_007 | 1772 | CCAGAGCACCAGAGCCATGGA |
| TGFBR2 | RNA0459_gTGFBR2_008 | 1773 | CCAGGTTGAACTCAGCTTCTG |
| TGFBR2 | RNA0460_gTGFBR2_009 | 1774 | CCCACCAGGGTGTCCAGCTCA |
| TGFBR2 | RNA0461_gTGFBR2_010 | 1775 | CTGAGGTCTATAAGGCCAAGC |
| TGFBR2 | RNA0462_gTGFBR2_011 | 1776 | AGACAGTGGCAGTCAAGATCT |
| TGFBR2 | RNA0463_gTGFBR2_012 | 1777 | CCTATGAGGAGTATGCCTCTT |
| TGFBR2 | RNA0464_gTGFBR2_013 | 1778 | CCCAACTCCGTCTTCCGCTCC |
| TGFBR2 | RNA0465_gTGFBR2_014 | 1779 | GGCTTTCCCTGCGTCTGGACC |
| TGFBR2 | RNA0466_gTGFBR2_015 | 1780 | CCTGCGTCTGGACCCTACTCT |
| TWF1 | RNA0285_gTWF1_012 | 1599 | ATAGAGCAACTTGTGATTGGA |
| TWF1 | RNA0286_gTWF1_060 | 1600 | ATGTGATGACTTTAATCAGTA |
| TWF1 | RNA0287_gTWF1_020 | 1601 | GAGGTGGCCACATTAAAGATG |
| TWF1 | RNA0288_gTWF1_053 | 1602 | TGAAGAAGTACATCCCAAGCA |
| TWF1 | RNA0289_gTWF1_101 | 1603 | AAATAGGTGGGCTACCTTTCT |
| TWF1 | RNA0290_gTWF1_018 | 1604 | ATGTGGCCACCTCCAAATTCC |
| TWF1 | RNA0291_gTWF1_022 | 1605 | ATCTGTCGTAGTTCTTCCTCA |
| TWF1 | RNA0292_gTWF1_015 | 1606 | CCCCTGTTGGAGGACAAACAA |
| TWF1 | RNA0293_gTWF1_005 | 1607 | CACAGCAAGTGAAGATGTTAA |
| TWF1 | RNA0294_gTWF1_051 | 1608 | CAGATCGAGATAGACAATGGG |

TABLE 7
Selected spacer sequences targeting human genes

| Target Gene | Name | SEQ ID NO | Spacer Sequence |
|---|---|---|---|
| ADORA2A | gADORA2A_12 | 983 | AGGATGTGGTCCCCATGAACT |
| B2M | gB2M_41 | 1302 | ATAGATCGAGACATGTAAGCA |
| CARD11 | gCARD11_1 | 1388 | TAGTACCGCTCCTGGAAGGTT |
| CD247 | gCD247_12 | 89 | CTAGCAGAGAAGGAAGAACCC |
| CD52 | gCD52_1 | 985 | CTCTTCCTCCTACTCACCATC |
| CIITA | gCIITA_32 | 1303 | CCTTGGGGCTCTGACAGGTAG |
| CTLA4 | gCTLA4_4 | 116 | AGCGGCACAAGGCTCAGCTGA |
| DCK | gDCK_6 | 1433 | CGGAGGCTCCTTACCGATGTT |
| FAS | gFAS_36 | 987 | GTGTTGCTGGTGAGTGTGCAT |
| HAVCR2 | gTIM3_6 | 333 | CTTGTAAGTAGTAGCAGCAGC |
| IL7R | gIL7R_3 | 1393 | CAGGGGAGATGGATCCTATCT |
| LAG3 | gLAG3_6 | 153 | GGGTGCATACCTGTCTGGCTG |
| LCK | gLCK1_3 | 1401 | ACCCATCAACCCGTAGGGATG |
| PDCD1 | gPD_23 | 210 | TCTGCAGGGACAATAGGAGCC |
| PLCG1 | gPLCG1_2 | 1403 | CCTTTCTGCGCTTCGTGGTGT |
| PTPN6 | gPTPN6_6 | 249 | TATGACCTGTATGGAGGGGAG |
| TIGIT | gTIGIT_2 | 302 | AGGCCTTACCTGAGGCGAGGG |
| TRAC | gTRAC006 | 988 | TGAGGGTGAAGGATAGACGCT |
| TRBC1 + 2 | gTRBC1 + 2_3 | 1373 | CGCTGTCAAGTCCAGTTCTAC |
| TRBC2 | gTRBC2_12 | 1379 | CCGGAGGTGAAGCCACAGTCT |

TABLE 8
Selected spacer sequences targeting human genes

| Target Gene | Name | SEQ ID NO | Spacer Sequence |
|---|---|---|---|
| ADORA2A | gADORA2A_12 | 983 | AGGATGTGGTCCCCATGAACT |
| B2M | gB2M_7 | 989 | ACTTTCCATTCTCTGCTGGAT |
| B2M | gB2M_30 | 1292 | AGTGGGGGTGAATTCAGTGTA |
| B2M | gB2M_41 | 1302 | ATAGATCGAGACATGTAAGCA |
| B2M | gB2M_4 | 984 | CTCACGTCATCCAGCAGAGAA |
| B2M | gB2M_17 | 991 | TATCTCTTGTACTACACTGAA |
| B2M | gB2M_2 | 990 | TGGCCTGGAGGCTATCCAGCG |
| CD247 | gCD247_19 | 99 | ACTCCCAAACAACCAGCGCCG |
| CD247 | gCD247_15 | 95 | ATCCCAATCTCACTGTAGGCC |
| CD247 | gCD247_3 | 105 | CGGAGGGTCTACGGCGAGGCT |
| CD247 | gCD247_12 | 89 | CTAGCAGAGAAGGAAGAACCC |
| CD247 | gCD247_8 | 110 | GACAAGAGACGTGGCCGGGAC |
| CD247 | gCD247_18 | 98 | TCATTTCACTCCCAAACAACC |
| CD247 | gCD247_1 | 90 | TGTGTTGCAGTTCAGCAGGAG |
| CD247 | gCD247_4 | 106 | TTATCTGTTATAGGAGCTCAA |
| CD3E | gCD3E_24 | 1795 | AGATCCAGGATACTGAGGGCA |
| CD3E | gCD3E_34 | 1796 | CTTCCTCTGGGGTAGCAGACA |
| CD40LG | gCD40LG_40 | 1798 | CTGCTGGCCTCACTTATGACA |
| CD52 | gCD52_1 | 985 | CTCTTCCTCCTACTCACCATC |
| CIITA | gCIITA_71 | 1341 | AAAGCCAAGTCCCTGAAGGAT |
| CIITA | gCIITA_33 | 1304 | ACCTTGGGGCTCTGACAGGTA |
| CIITA | gCIITA_59 | 1329 | AGAGCTCAGGGATGACAGAGC |
| CIITA | gCIITA_80 | 1349 | CAAGGACTTCAGCTGGGGGAA |
| CIITA | gCIITA_57 | 1327 | CAGAAGAAGCTGCTCCGAGGT |
| CIITA | gCIITA_70 | 1340 | CCAGGTCTTCCACATCCTTCA |
| CIITA | gCIITA_32 | 1303 | CCTTGGGGCTCTGACAGGTAG |
| CIITA | gCIITA_82 | 1351 | CGACAGCTTGTACAATAACTG |
| CIITA | gCIITA_35 | 1306 | CTCCCAGAACCCGACACAGAC |
| CIITA | gCIITA_48 | 1319 | CTCGGGAGGTCAGGGCAGGTT |
| CIITA | gCIITA_38 | 1309 | CTTGTCTGGGCAGCGGAACTG |
| CIITA | gCIITA_65 | 1335 | GCAGCACGTGGTACAGGAGCT |
| CIITA | gCIITA_63 | 1333 | GCCACTCAGAGCCAGCCACAG |
| CIITA | gCIITA_76 | 1346 | GGGAAAGCCTGGGGGCCTGAG |
| CITTA | gCIITA_72 | 1342 | GGTCCCGAACAGCAGGGAGCT |
| CIITA | gCIITA_81 | 1350 | TAGGCACCCAGGTCAGTGATG |
| CIITA | gCIITA_4 | 986 | TAGGGGCCCCAACTCCATGGT |
| CIITA | gCIITA_40 | 1311 | TCAAAGTAGAGCACATAGGAC |
| CIITA | gCIITA_44 | 1315 | TCCAGGCGCATCTGGCCGGAG |
| CIITA | gCIITA_43 | 1314 | TCTGCAGCCTTCCCAGAGGAG |
| CIITA | gCIITA_41 | 1312 | TGCCCAACTTCTGCTGGCATC |
| CIITA | gCIITA_60 | 1330 | TGCCGGGCAGTGTGCCAGCTC |
| CIITA | gCIITA_67 | 1337 | TGGGCACCCGCCTCACGCCTC |
| CIITA | gCIITA_36 | 1307 | TGGGCTCAGGTGCTTCCTCAC |
| CIITA | gCIITA_73 | 1343 | TTTAGGTCCCGAACAGCAGGG |
| CSF2 | gCSF2_007 | 1797 | CACAGGAGCCGACCTGCCTAC |
| CTLA4 | gCTLA4_4 | 116 | AGCGGCACAAGGCTCAGCTGA |
| CTLA4 | gCTLA4_19 | 114 | CACTGGAGGTGCCCGTGCAGA |
| CTLA4 | gCTLA4_6 | 113 | CAGAAGACAGGGATGAAGAGA |
| CTLA4 | gCTLA4_14 | 112 | CCTGGAGATGCATACTCACAC |
| CTLA4 | gCTLA4_13 | 115 | TGTGTGAGTATGCATCTCCAG |
| DCK | gDCK_26 | 994 | AGCTTGCCATTCAGAGAGGCA |
| DCK | gDCK_6 | 1433 | CGGAGGCTCCTTACCGATGTT |
| DCK | gDCK_8 | 993 | CTCACAACAGCTGCAGGGAAG |
| DCK | gDCK_30 | 995 | TACATACCTGTCACTATACAC |
| DCK | gDCK_2 | 992 | TCAGCCAGCTCTGAGGGGACC |
| FAS | gFAS_35 | 997 | ATGATTCCATGTTCACATCTA |
| FAS | gFAS_1 | 999 | GGAGGATTGCTCAACAACCAT |
| FAS | gFAS_12 | 998 | GTGTAACATACCTGGAGGACA |
| FAS | gFAS_36 | 987 | GTGTTGCTGGTGAGTGTGCAT |
| FAS | gFAS_59 | 1000 | TAGGAAACAGTGGCAATAAAT |
| FAS | gFAS_34 | 996 | TTTTTCTAGATGTGAACATGG |
| HAVCR2 | gTIM3_12 | 337 | AATGTGGCAACGTGGTGCTCA |
| HAVCR2 | gTIM3_29 | 334 | CAAGGATGCTTACCACCAGGG |
| HAVCR2 | gTIM3_30 | 336 | CCACCAGGGGACATGGCCCAG |
| HAVCR2 | gTIM3_18 | 345 | CGCAAAGGAGATGTGTCCCTG |
| HAVCR2 | gTIM3_6 | 333 | CTTGTAAGTAGTAGCAGCAGC |
| HAVCR2 | gTIM3_6 | 335 | TAAGTAGTAGCAGCAGCAGCA |
| HAVCR2 | gTIM3_32 | 359 | TATCAGGGAGGCTCCCCAGTG |
| HAVCR2 | gTIM3_25 | 353 | TGACATTAGCCAAGGTCACCC |
| IL7R | gIL7R_3 | 1393 | CAGGGGAGATGGATCCTATCT |
| IL7R | gIL7R_8 | 1398 | CATAACACACAGGCCAAGATG |
| LAG3 | gLAG3_6 | 153 | GGGTGCATACCTGTCTGGCTG |
| LAG3 | gLAG3_33 | 154 | GGTCACCTGGATCCCTGGGGA |
| LAG3 | gLAG3_38 | 155 | TCAGGACCTTGGCTGGAGGCA |
| LCK | gLCK1_3 | 1401 | ACCCATCAACCCGTAGGGATG |
| PDCD1 | gPD_27 | 211 | CAGTGGCGAGAGAAGACCCCG |
| PDCD1 | gPD_2 | 224 | CCTTCCGCTCACCTCCGCCTG |
| PDCD1 | gPD_29 | 212 | CTAGCGGAATGGGCACCTCAT |
| PDCD1 | gPD_8 | 209 | GCACGAAGCTCTCCGATGTGT |
| PDCD1 | gPD_23 | 210 | TCTGCAGGGACAATAGGAGCC |
| PTPN6 | gPTPN6_22 | 268 | AAGAAGACGGGGATTGAGGAG |
| PTPN6 | gPTPN6_1 | 254 | ACCGAGACCTCAGTGGGCTGG |
| PTPN6 | gPTPN6_46 | 252 | ACTGCCCCCCACCCAGGCCTG |
| PTPN6 | gPTPN6_26 | 251 | CAGAAGCAGGAGGTGAAGAAC |
| PTPN6 | gPTPN6_25 | 271 | CCCACCCACATCTCAGAGTTT |
| PTPN6 | gPTPN6_7 | 250 | CGACTCTGACAGAGCTGGTGG |
| PTPN6 | gPTPN6_19 | 264 | GCTCCCCCCAGGGTGGACGCT |
| PTPN6 | gPTPN6_14 | 259 | GGCTGGTCACTGAGCACAGAA |
| PTPN6 | gPTPN6_6 | 249 | TATGACCTGTATGGAGGGGAG |
| PTPN6 | gPTPN6_5 | 293 | TCCCCTCCATACAGGTCATAG |
| PTPN6 | gPTPN6_37 | 253 | TGGGCCCTACTCTGTGACCAA |
| PTPN6 | gPTPN6_16 | 261 | TGTGCTCAGTGACCAGCCCAA |
| PTPN6 | gPTPN6_12 | 257 | TTGTGCGTGAGAGCCTCAGCC |
| TIGIT | gTIGIT_2 | 302 | AGGCCTTACCTGAGGCGAGGG |
| TIGIT | gTIGIT_18 | 303 | GTCCTCCCTCTAGTGGCTGAG |
| TRAC | gTRAC079 | 1006 | ATTCCTCCACTTCAACACCTG |
| TRAC | gTRAC017 | 1434 | CAGGTGAAATTCCTGAGATGT |
| TRAC | gTRAC078 | 1002 | CCAGCTCACTAAGTCAGTCTC |
| TRAC | gTRAC082 | 1016 | CCAGCTGACAGATGGGCTCCC |
| TRAC | gTRAC028 | 1022 | CCATGCCTGCCTTTACTCTGC |
| TRAC | gTRAC041 | 1018 | CCCCAACCCAGGCTGGAGTCC |
| TRAC | gTRAC040 | 1017 | CCGTATAAAGCATGAGACCGT |
| TRAC | gTRAC067 | 1005 | CCGTGTCATTCTCTGGACTGC |
| TRAC | gTRAC018 | 1013 | CTCGATATAAGGCCTTGAGCA |
| TRAC | gTRAC029 | 1021 | CTCTGCCAGAGTTATATTGCT |
| TRAC | gTRAC058 | 1009 | CTTGCTTCAGGAATGGCCAGG |
| TRAC | gTRAC059 | 1435 | GACATCATTGACCAGAGCTCT |
| TRAC | gTRAC043 | 1014 | GAGTCTCTCAGCTGGTACACG |
| TRAC | gTRAC073 | 1001 | GCAGACAGGGAGAAATAAGGA |
| TRAC | gTRAC074 | 1012 | GGCAGACAGGGAGAAATAAGG |
| TRAC | gTRAC050 | 1023 | GTCTGTGATATACACATCAGA |
| TRAC | gTRAC061 | 1008 | GTGGCAATGGATAAGGCCGAG |
| TRAC | gTRAC039 | 1004 | TAAGATGCTATTTCCCGTATA |
| TRAC | gTRAC038 | 1007 | TACGGGAAATAGCATCTTAGA |
| TRAC | gTRAC021 | 1010 | TAGTTCAAAACCTCTATCAAT |
| TRAC | gTRAC012 | 1003 | TATGGAGAAGCTCTCATTTCT |
| TRAC | gTRAC014 | 1020 | TCAGAAGAGCCTGGCTAGGAA |
| TRAC | gTRAC049 | 1011 | TCTGTGATATACACATCAGAA |
| TRAC | gTRAC006 | 988 | TGAGGGTGAAGGATAGACGCT |
| TRAC | gTRAC075 | 1015 | TGGCAGACAGGGAGAAATAAG |
| TRAC | gTRAC076 | 1019 | TTGGCAGACAGGGAGAAATAA |
| TRBC1 + 2 | gTRBC1 + 2_1 | 1372 | AGCCATCAGAAGCAGAGATCT |
| TRBC1 + 2 | gTRBC1 + 2_3 | 1373 | CGCTGTCAAGTCCAGTTCTAC |
| TRBC1 + 2 | gTRBC1_3_001 | 1794 | GGTGTGGGAGATCTCTGCTTC |
| TRBC2 | gTRBC2_11 | 1378 | AGACTGTGGCTTCACCTCCGG |
| TRBC2 | gTRBC2_12 | 1379 | CCGGAGGTGAAGCCACAGTCT |
| TRBC2 | gTRBC2_15 | 1382 | CTAGGGAAGGCCACCTTGTAT |
| TRBC2 | gTRBC2_21 | 1387 | GAGCTAGCCTCTGGAATCCTT |

TABLE 9
Selected spacer sequences targeting human genes

| Target Gene | Name | SEQ ID NO | Spacer Sequence |
|---|---|---|---|
| ADORA2A | gADORA2A_28 | 1025 | AAGGCAGCTGGCACCAGTGCC |
| ADORA2A | gADORA2A_4 | 1030 | CCATCACCATCAGCACCGGGT |
| ADORA2A | gADORA2A_8 | 1029 | CCATCGGCCTGACTCCCATGC |
| ADORA2A | gADORA2A_16 | 1024 | CGGATCTTCCTGGCGGCGCGA |
| ADORA2A | gADORA2A_7 | 1028 | GTGACCGGCACGAGGGCTAAG |
| ADORA2A | gADORA2A_2 | 1026 | TGGTGTCACTGGCGGCGGCCG |
| ADORA2A | gADORA2A_23 | 1027 | TTCTGCCCCGACTGCAGCCAC |
| B2M | gB2M_27 | 1289 | AATTCTCTCTCCATTCTTCAG |
| B2M | gB2M_10 | 1036 | ATCCATCCGACATTGAAGTTG |
| B2M | gB2M_31 | 1293 | CAGTGGGGGTGAATTCAGTGT |
| B2M | gB2M_40 | 1301 | CATAGATCGAGACATGTAAGC |
| B2M | gB2M_5 | 1035 | CATTCTCTGCTGGATGACGTG |
| B2M | gB2M_22 | 1037 | CCCCACTTAACTATCTTGGGC |
| B2M | gB2M_11 | 1033 | CTGAAGAATGGAGAGAGAATT |
| B2M | gB2M_8 | 1032 | CTGAATTGCTATGTGTCTGGG |
| B2M | gB2M_1 | 1038 | GCTGTGCTCGCGCTACTCTCT |
| B2M | gB2M_21 | 1031 | TCACAGCCCAAGATAGTTAAG |
| B2M | gB2M_18 | 1034 | TCAGTGGGGGTGAATTCAGTG |
| CD247 | gCD247_7 | 109 | CCCCCATCTCAGGGTCCCGGC |
| CD247 | gCD247_22 | 103 | CTTTCACGCCAGGGTCTCAGT |
| CD247 | gCD247_9 | 111 | TCTCCCTCTAACGTCTTCCCG |
| CD247 | gCD247_21 | 102 | TGATTTGCTTTCACGCCAGGG |
| CD247 | gCD247_14 | 94 | TGCAGGAACTGCAGAAAGATA |
| CD247 | gCD247_13 | 93 | TGCAGTTCCTGCAGAAGAGGG |
| CD52 | gCD52_4 | 1039 | GCTGGTGTCGTTTTGTCCTGA |
| CIITA | gCIITA_55 | 1325 | AGCCACATCTTGAAGAGACCT |
| CIITA | gCIITA_58 | 1328 | AGCTGTCCGGCTTCTCCATGG |
| CIITA | gCIITA_51 | 1322 | CAGAGCCGGTGGAGCAGTTCT |
| CIITA | gCIITA_46 | 1317 | CCAGAGCCCATGGGGCAGAGT |
| CIITA | gCIITA_52 | 1323 | CCCAGCACAGCAATCACTCGT |
| CIITA | gCIITA_68 | 1338 | CCCCTCTGGATTGGGGAGCCT |
| CIITA | gCIITA_34 | 1305 | CCGGCCTTTTTACCTTGGGGC |
| CIITA | gCIITA_75 | 1345 | CCTCCTAGGCTGGGCCCTGTC |
| CIITA | gCIITA_29 | 1041 | GTCTCTTGCAGTGCCTTTCTC |
| CIITA | gCIITA_47 | 1318 | TCCCCACCATCTCCACTCTGC |
| CIITA | gCIITA_83 | 1352 | TCTTGCCAGCGTCCAGTACAA |
| CIITA | gCIITA_42 | 1313 | TGACTTTTCTGCCCAACTTCT |
| CIITA | gCIITA_18 | 1040 | TGCTGGCATCTCCATACTCTC |
| CTLA4 | gCTLA4_36 | 143 | ACAGCTAAAGAAAAGAAGCCC |
| CTLA4 | gCTLA4_37 | 144 | CACATAGACCCCTGTTGTAAG |
| CTLA4 | gCTLA4_18 | 124 | CTAGATGATTCCATCTGCACG |
| CTLA4 | gCTLA4_28 | 134 | CTCCTCTGGATCCTTGCAGCA |
| CTLA4 | gCTLA4_27 | 133 | CTGTTGCAGATCCAGAACCGT |
| CTLA4 | gCTLA4_41 | 148 | TCAATTGATGGGAATAAAATA |
| CTLA4 | gCTLA4_5 | 149 | TTCTTCTCTTCATCCCTGTCT |
| DCK | gDCK_9 | 1042 | AGGATATTCACAAATGTTGAC |
| DCK | gDCK_7 | 1045 | ATCTTTCCTCACAACAGCTGC |
| DCK | gDCK_22 | 1043 | GAAGGTAAAAGACCATCGTTC |
| DCK | gDCK_21 | 1044 | TCATACATCATCTGAAGAACA |
| FAS | gFAS_4 | 1058 | ACAGGTTCTTACGTCTGTTGC |
| FAS | gFAS_47 | 1046 | AGTGAAGAGAAAGGAAGTACA |
| FAS | gFAS_25 | 1048 | CTAGGCTTAGAAGTGGAAATA |
| FAS | gFAS_38 | 1056 | CTCTTTGCACTTGGTGTTGCT |
| FAS | gFAS_71 | 1055 | CTGTTCTGCTGTGTCTTGGAC |
| FAS | gFAS_33 | 1054 | CTTGGTGCAAGGGTCACAGTG |
| FAS | gFAS_10 | 1049 | GAAGGCCTGCATCATGATGGC |
| FAS | gFAS_5 | 1051 | GGACGATAATCTAGCAACAGA |
| FAS | gFAS_15 | 1059 | GGCAGGTGAAAGGAAAGCTAG |
| FAS | gFAS_32 | 1050 | GTGCAAGGGTCACAGTGTTCA |
| FAS | gFAS_29 | 1053 | GTTTACATCTGCACTTGGTAT |
| FAS | gFAS_70 | 1057 | TGTTCTGCTGTGTCTTGGACA |
| FAS | gFAS_14 | 1052 | TTCCTTGGGCAGGTGAAAGGA |
| FAS | gFAS_45 | 1047 | TTTGTTCTTTCAGTGAAGAGA |
| HAVCR2 | gTIM3_23 | 351 | ACCTGAAGTTGGTCATCAAAC |
| HAVCR2 | gTIM3_27 | 355 | ACTGCAGCCTTTCCAAGGATG |
| HAVCR2 | gTIM3_13 | 340 | ATCAGTCCTGAGCACCACGTT |
| HAVCR2 | gTIM3_28 | 356 | CCAAGGATGCTTACCACCAGG |
| HAVCR2 | gTIM3_48 | 376 | CCAATCCTGAGGGAGGGAGGT |
| HAVCR2 | gTIM3_10 | 383 | CCCCAGCAGACGGGCACGAGG |
| HAVCR2 | gTIM3_41 | 369 | CCCCTTACTAGGGTATTCTCA |
| HAVCR2 | gTIM3_36 | 363 | CGGGACTCTGGAGCAACCATC |
| HAVCR2 | gTIM3_42 | 370 | CTAGGGTATTCTCATAGCAAA |
| HAVCR2 | gTIM3_19 | 346 | GATCCGGCAGCAGTAGATCCC |
| HAVCR2 | gTIM3_47 | 375 | GCCAACCTCCCTCCCTCAGGA |
| HAVCR2 | gTIM3_15 | 342 | GCCAGTATCTGGATGTCCAAT |
| HAVCR2 | gTIM3_40 | 367 | GTTTCCCCCTTACTAGGGTAT |
| HAVCR2 | gTIM3_34 | 361 | TGTTTCCATAGCAAATATCCA |
| IL7R | gIL7R_2 | 1392 | CCAGGGGAGATGGATCCTATC |
| IL7R | gIL7R_7 | 1397 | TCTGTCGCTCTGTTGGTCATC |
| LAG3 | gLAG3_3 | 180 | ACCTGGAGCCACCCAAAGCGG |
| LAG3 | gLAG3_27 | 177 | CCACCTGAGGCTGACCTGTGA |
| LAG3 | gLAG3_41 | 156 | CCAGCCTTGGCAATGCCAGCT |
| LAG3 | gLAG3_31 | 182 | CCCAGGGATCCAGGTGACCCA |
| LAG3 | gLAG3_25 | 175 | CCCTTCGACTAGAGGATGTGA |
| LAG3 | gLAG3_13 | 162 | CGCTAAGTGGTGATGGGGGGA |
| LAG3 | gLAG3_22 | 172 | GCAGTGAGGAAAGACCGGGTC |
| LAG3 | gLAG3_16 | 165 | GGGCAGGAAGAGGAAGCTTTC |
| LAG3 | gLAG3_46 | 194 | TCCATAGGTGCCCAACGCTCT |
| LAG3 | gLAG3_35 | 157 | TGAGGTGACTCCAGTATCTGG |
| LAG3 | gLAG3_37 | 186 | TGTGGAGCTCTCTGGACACCC |
| PDCD1 | gPD_20 | 225 | CAGAGAGAAGGGCAGAAGTGC |
| PDCD1 | gPD_22 | 227 | GAACTGGCCGGCTGGCCTGGG |
| PDCD1 | gPD_18 | 222 | GTGCCCTTCCAGAGAGAAGGG |
| PLCG1 | gPLCG1_2 | 1403 | CCTTTCTGCGCTTCGTGGTGT |
| PLCG1 | gPLCG1_5 | 1406 | GTGGTGTATGAGGAAGACATG |
| PLCG1 | gPLCG1_4 | 1405 | TGCGCTTCGTGGTGTATGAGG |
| PTPN6 | gPTPN6_48 | 291 | AATGAACTGGGCGATGGCCAC |
| PTPN6 | gPTPN6_8 | 300 | AGGTGGATGATGGTGCCGTCG |
| PTPN6 | gPTPN6_28 | 273 | CACCAGCGTCTGGAAGGGCAG |
| PTPN6 | gPTPN6_39 | 283 | CAGGTCTCCCCGCTGGACAAT |
| PTPN6 | gPTPN6_53 | 295 | CCCCCCTGCACCCGGCTGCAG |
| PTPN6 | gPTPN6_42 | 287 | CTGCCGCTGGTTGATCTGGTC |
| PTPN6 | gPTPN6_41 | 286 | CTGGACCAGATCAACCAGCGG |
| PTPN6 | gPTPN6_4 | 284 | CTGGCTCGGCCCAGTCGCAAG |
| PTPN6 | gPTPN6_20 | 266 | GAGACCTTCGACAGCCTCACG |
| PTPN6 | gPTPN6_40 | 285 | GGGAGACCTGATTCGGGAGAT |
| PTPN6 | gPTPN6_10 | 255 | TCTAGGTGGTACCATGGCCAC |
| PTPN6 | gPTPN6_32 | 277 | TGGCAGATGGCGTGGCAGGAG |
| TIGIT | gTIGIT_27 | 322 | CTCCTGAGGTCACCTTCCACA |
| TIGIT | gTIGIT_11 | 304 | GGGTGGCACATCTCCCCATCC |
| TIGIT | gTIGIT_10 | 306 | TAATGCTGACTTGGGGTGGCA |
| TIGIT | gTIGIT_7 | 305 | TGCAGAGAAAGGTGGCTCTAT |
| TRAC | gTRAC019 | 1070 | AACTATAAATCAGAACACCTG |
| TRAC | gTRAC044 | 1063 | AGAATCAAAATCGGTGAATAG |
| TRAC | gTRAC035 | 1062 | AGGTTTCCTTGAGTGGCAGGC |
| TRAC | gTRAC007 | 1081 | ATAAACTGTAAAGTACCAAAC |
| TRAC | gTRAC030 | 1077 | ATAGGATCTTCTTCAAAACCC |
| TRAC | gTRAC048 | 1071 | ATTCTCAAACAAATGTGTCAC |
| TRAC | gTRAC056 | 1073 | CATGTGCAAACGCCTTCAACA |
| TRAC | gTRAC083 | 1083 | CCCAGCTGACAGATGGGCTCC |
| TRAC | gTRAC072 | 1064 | CCCCTTACTGCTCTTCTAGGC |
| TRAC | gTRAC068 | 1068 | CCCGTGTCATTCTCTGGACTG |
| TRAC | gTRAC042 | 1061 | CCTCTTTGCCCCAACCCAGGC |
| TRAC | gTRAC066 | 1060 | CTAAGAAACAGTGAGCCTTGT |
| TRAC | gTRAC071 | 1075 | CTCAGACTGTTTGCCCCTTAC |
| TRAC | gTRAC025 | 1069 | CTGGGCCTTTTTCCCATGCCT |
| TRAC | gTRAC036 | 1072 | CTTGAGTGGCAGGCCAGGCCT |
| TRAC | gTRAC020 | 1066 | GAACTATAAATCAGAACACCT |
| TRAC | gTRAC033 | 1078 | GAAGAAGATCCTATTAAATAA |
| TRAC | gTRAC084 | 1082 | GACTTTTCCCAGCTGACAGAT |
| TRAC | gTRAC062 | 1065 | GGTGGCAATGGATAAGGCCGA |
| TRAC | gTRAC009 | 1080 | GTACTTTACAGTTTATTAAAT |
| TRAC | gTRAC081 | 1076 | TAATTCCTCCACTTCAACACC |
| TRAC | gTRAC064 | 1074 | TACTAAGAAACAGTGAGCCTT |
| TRAC | gTRAC001 | 1079 | TGTTTTTAATGTGACTCTCAT |
| TRAC | gTRAC013 | 1067 | TTTCTCAGAAGAGCCTGGCTA |
| TRBC2 | gTRBC2_19 | 1386 | CACAGGTCAAGAGAAAGGATT |
| TRBC2 | gTRBC2_14 | 1381 | CCAGCAAGGGGTCCTGTCTGC |
| TRBC2 | gTRBC2_17 | 1384 | CCATGGCCATCAGCACGAGGG |

The spacer sequences provided in Tables 7-9 are designed based upon identification of target nucleotide sequences associated with a PAM in a given target gene locus, and are selected based upon the editing efficiency detected in human cells. Further exemplary spacer sequences useful in embodiments of the methods and compositions disclosed herein are shown in Table 10.

TABLE 10
Tested crRNAs targeting human genes

| Target | | SEQ | | |
|---|---|---|---|---|
| Gene | Name | ID NO | Spacer Sequence | % Indel |
| ADORA2A | gADORA2A_26 | 1102 | AAAGGTTCTTGCTGCCTCAGG | 0.1 |
| ADORA2A | gADORA2A_13 | 1091 | AACTTCTTTGCCTGTGTGCTG | 0.1 |
| ADORA2A | gADORA2A_28 | 1025 | AAGGCAGCTGGCACCAGTGCC | 5.8 |
| ADORA2A | gADORA2A_21 | 1098 | ACTTTCTTCTGCCCCGACTGC | 0.6 |
| ADORA2A | gADORA2A_29 | 1104 | AGCTCATGGCTAAGGAGCTCC | 0.2 |
| ADORA2A | gADORA2A_17 | 1094 | AGCTGTCGTCGCGCCGCCAGG | 0.1 |
| ADORA2A | gADORA2A_12 | 983 | AGGATGTGGTCCCCATGAACT | 18.2 |
| ADORA2A | gADORA2A_24 | 1100 | ATCTACGCCTACCGTATCCGC | 0 |
| ADORA2A | gADORA2A_27 | 1103 | CAAGGCAGCTGGCACCAGTGC | 0.1 |
| ADORA2A | gADORA2A_4 | 1030 | CCATCACCATCAGCACCGGGT | 2.1 |
| ADORA2A | gADORA2A_8 | 1029 | CCATCGGCCTGACTCCCATGC | 2.2 |
| ADORA2A | gADORA2A_20 | 1097 | CCCTCTGCTGGCTGCCCCTAC | 0.6 |
| ADORA2A | gADORA2A_15 | 1093 | CCTGTGTGCTGGTGCCCCTGC | 1.1 |
| ADORA2A | gADORA2A_25 | 1101 | CGCAAGATCATTCGCAGCCAC | 0.1 |
| ADORA2A | gADORA2A_16 | 1024 | CGGATCTTCCTGGCGGCGCGA | 7.8 |
| ADORA2A | gADORA2A_22 | 1099 | CTTCTGCCCCGACTGCAGCCA | 1 |
| ADORA2A | gADORA2A_19 | 1096 | GCAGCATGGACCTCCTTCTGC | 0.4 |
| ADORA2A | gADORA2A_3 | 1085 | GCCATCACCATCAGCACCGGG | 0.5 |
| ADORA2A | gADORA2A_30 | 1105 | GCCATGAGCTCAAGGGAGTGT | 0.5 |
| ADORA2A | gADORA2A_11 | 1090 | GCCCTCCCCGCAGCCCTGGGA | 1.3 |
| ADORA2A | gADORA2A_6 | 1087 | GCCCTCGTGCCGGTCACCAAG | 0.9 |
| ADORA2A | gADORA2A_9 | 1088 | GCTGACCGCAGTTGTTCCAAC | 1.1 |
| ADORA2A | gADORA2A_10 | 1089 | GGCTGACCGCAGTTGTTCCAA | 0.5 |
| ADORA2A | gADORA2A_5 | 1086 | GTCCTGGTCCTCACGCAGAGC | 0.1 |
| ADORA2A | gADORA2A_7 | 1028 | GTGACCGGCACGAGGGCTAAG | 2.8 |
| ADORA2A | gADORA2A_1 | 1084 | GTGGTGTCACTGGCGGCGGCC | 0.3 |
| ADORA2A | gADORA2A_18 | 1095 | TGCAGTGTGGACCGTGCCCGC | 0.2 |
| ADORA2A | gADORA2A_2 | 1026 | TGGTGTCACTGGCGGCGGCCG | 3.9 |
| ADORA2A | gADORA2A_23 | 1027 | TTCTGCCCCGACTGCAGCCAC | 2.8 |
| ADORA2A | gADORA2A_14 | 1092 | TTTGCCTGTGTGCTGGTGCCC | 0.2 |
| B2M | gB2M_9 | 1108 | AATGTCGGATGGATGAAACCC | 0.5 |
| B2M | gB2M_27 | 1289 | AATTCTCTCTCCATTCTTCAG | 2.7 |
| B2M | gB2M_19 | 1114 | ACTATCTTGGGCTGTGACAAA | 0.1 |
| B2M | gB2M_7 | 989 | ACTTTCCATTCTCTGCTGGAT | 17.9 |
| B2M | gB2M_16 | 1113 | AGCAAGGACTGGTCTTTCTAT | 0.3 |
| B2M | gB2M_26 | 1288 | AGTAAGTCAACTTCAATGTCG | 0.11 |
| B2M | gB2M_30 | 1292 | AGTGGGGGTGAATTCAGTGTA | 91.96 |
| B2M | gB2M_41 | 1302 | ATAGATCGAGACATGTAAGCA | 93.92 |
| B2M | gB2M_10 | 1036 | ATCCATCCGACATTGAAGTTG | 2 |
| B2M | gB2M_36 | 1297 | CAAAAGAATGTAAGACTTACC | 0.13 |
| B2M | gB2M_28 | 1290 | CAATTCTCTCTCCATTCTTCA | 0.26 |
| B2M | gB2M_29 | 1291 | CAGCAAGGACTGGTCTTTCTA | 0.19 |
| B2M | gB2M_31 | 1293 | CAGTGGGGGTGAATTCAGTGT | 8.1 |
| B2M | gB2M_40 | 1301 | CATAGATCGAGACATGTAAGC | 4.25 |
| B2M | gB2M_5 | 1035 | CATTCTCTGCTGGATGACGTG | 2.2 |
| B2M | gB2M_6 | 1107 | CCATTCTCTGCTGGATGACGT | 1 |
| B2M | gB2M_22 | 1037 | CCCCACTTAACTATCTTGGGC | 2 |
| B2M | gB2M_3 | 1106 | CCCGATATTCCTCAGGTACTC | 0.1 |
| B2M | gB2M_25 | 1287 | CCGATATTCCTCAGGTACTCC | 0.14 |
| B2M | gB2M_37 | 1298 | CCTCCATGATGCTGCTTACAT | 0.81 |
| B2M | gB2M_33 | 1294 | CTATCTCTTGTACTACACTGA | 0.21 |
| B2M | gB2M_4 | 984 | CTCACGTCATCCAGCAGAGAA | 74.1 |
| B2M | gB2M_14 | 1111 | CTGAAAGACAAGTCTGAATGC | 0.4 |
| B2M | gB2M_11 | 1033 | CTGAAGAATGGAGAGAGAATT | 3.4 |
| B2M | gB2M_8 | 1032 | CTGAATTGCTATGTGTCTGGG | 3.5 |
| B2M | gB2M_23 | 1285 | CTGGCCTGGAGGCTATCCAGC | 0.77 |
| B2M | gB2M_1 | 1038 | GCTGTGCTCGCGCTACTCTCT | 1.8 |
| B2M | gB2M_35 | 1296 | GGCTGTGACAAAGTCACATGG | 0.18 |
| B2M | gB2M_20 | 1115 | GTCACAGCCCAAGATAGTTAA | 0.8 |
| B2M | gB2M_34 | 1295 | TACTACACTGAATTCACCCCC | 0.8 |
| B2M | gB2M_17 | 991 | TATCTCTTGTACTACACTGAA | 15.3 |
| B2M | gB2M_12 | 1109 | TCAATTCTCTCTCCATTCTTC | 0.7 |
| B2M | gB2M_21 | 1031 | TCACAGCCCAAGATAGTTAAG | 5.3 |
| B2M | gB2M_18 | 1034 | TCAGTGGGGGTGAATTCAGTG | 3 |
| B2M | gB2M_39 | 1300 | TCATAGATCGAGACATGTAAG | 0.2 |
| B2M | gB2M_24 | 1286 | TCCCGATATTCCTCAGGTACT | 0.54 |
| B2M | gB2M_15 | 1112 | TCTTTCAGCAAGGACTGGTCT | 0.9 |
| B2M | gB2M_2 | 990 | TGGCCTGGAGGCTATCCAGCG | 17.4 |
| B2M | gB2M_13 | 1110 | TTCAATTCTCTCTCCATTCTT | 0.7 |
| B2M | gB2M_38 | 1299 | TTCATAGATCGAGACATGTAA | 0.18 |
| CD52 | gCD52_6 | 1119 | CCTTTTCTTCGTGGCCAATGC | 0.2 |
| CD52 | gCD52_1 | 985 | CTCTTCCTCCTACTCACCATC | 28.4 |
| CD52 | gCD52_8 | 1121 | CTTCGTGGCCAATGCCATAAT | 0.15 |
| CD52 | gCD52_4 | 1039 | GCTGGTGTCGTTTTGTCCTGA | 4.1 |
| CD52 | gCD52_3 | 1117 | GTCCTGAGAGTCCAGTTTGTA | N.D. |
| CD52 | gCD52_2 | 1116 | TCCTCCTACAGATACAAACTG | N.D. |
| CD52 | gCD52_7 | 1120 | TCTTCGTGGCCAATGCCATAA | 0.2 |
| CD52 | gCD52_5 | 1118 | TGTTGCTGGATGCTGAGGGGC | 1.1 |
| CIITA | gCIITA_71 | 1341 | AAAGCCAAGTCCCTGAAGGAT | 39.5 |
| CIITA | gCIITA_69 | 1339 | AAAGGCTCGATGGTGAACTTC | 1.17 |
| CIITA | gCIITA_33 | 1304 | ACCTTGGGGCTCTGACAGGTA | 11.83 |
| CIITA | gCIITA_59 | 1329 | AGAGCTCAGGGATGACAGAGC | 16.35 |
| CIITA | gCIITA_55 | 1325 | AGCCACATCTTGAAGAGACCT | 5.71 |
| CIITA | gCIITA_58 | 1328 | AGCTGTCCGGCTTCTCCATGG | 3.25 |
| CIITA | gCIITA_24 | 1143 | AGGTCTGCCGGAAGCTCCTCT | 0.1 |
| CIITA | gCIITA_89 | 1357 | ATCACCTTCCATGTCACACAA | 0.31 |
| CIITA | gCIITA_17 | 1137 | ATCTGGTCCTATGTGCTCTAC | 0.2 |
| CIITA | gCIITA_61 | 1331 | ATGTCTGCGGCCCAGCTCCCA | 1.25 |
| CIITA | gCIITA_80 | 1349 | CAAGGACTTCAGCTGGGGGAA | 87.87 |
| CIITA | gCIITA_57 | 1327 | CAGAAGAAGCTGCTCCGAGGT | 12.02 |
| CIITA | gCIITA_51 | 1322 | CAGAGCCGGTGGAGCAGTTCT | 8.94 |
| CIITA | gCIITA_92 | 1360 | CAGGACTCCCAGCTGGAGGGC | 0.61 |
| CIITA | gCIITA_94 | 1362 | CAGTGCCTTTCTCCAGTTCCT | 0.25 |
| CIITA | gCIITA_25 | 1144 | CAGTGCTTCAGGTCTGCCGGA | 0.2 |
| CIITA | gCIITA_9 | 1129 | CATGTCACACAACAGCCTGCT | 0.1 |
| CIITA | gCIITA_56 | 1326 | CCAGAAGAAGCTGCTCCGAGG | 0.52 |
| CIITA | gCIITA_46 | 1317 | CCAGAGCCCATGGGGCAGAGT | 1.51 |
| CIITA | gCIITA_23 | 1142 | CCAGAGGAGCTTCCGGCAGAC | 0.9 |
| CIITA | gCIITA_70 | 1340 | CCAGGTCTTCCACATCCTTCA | 38.98 |
| CIITA | gCIITA_77 | 1347 | CCCAAACTGGTGCGGATCCTC | 0.57 |
| CIITA | gCIITA_52 | 1323 | CCCAGCACAGCAATCACTCGT | 2.63 |
| CIITA | gCIITA_68 | 1338 | CCCCTCTGGATTGGGGAGCCT | 4.61 |
| CIITA | gCIITA_84 | 1353 | CCCGGCCTTTTTACCTTGGGG | 0.38 |
| CIITA | gCIITA_34 | 1305 | CCGGCCTTTTTACCTTGGGGC | 2.26 |
| CIITA | gCIITA_8 | 1128 | CCTCCCAGAACCCGACACAGA | 0.1 |
| CIITA | gCIITA_85 | 1354 | CCTCCCAGGCAGCTCACAGTG | 0.74 |
| CIITA | gCIITA_75 | 1345 | CCTCCTAGGCTGGGCCCTGTC | 2.78 |
| CIITA | gCIITA_97 | 1365 | CCTGTCATGTTTGCTCGGGAG | 0.27 |
| CIITA | gCIITA_32 | 1303 | CCTTGGGGCTCTGACAGGTAG | 93.85 |
| CIITA | gCIITA_12 | 1132 | CCTTGTCTGGGCAGCGGAACT | 0.4 |
| CIITA | gCIITA_82 | 1351 | CGACAGCTTGTACAATAACTG | 34.37 |
| CIITA | gCIITA_26 | 1145 | CGGCAGACCTGAAGCACTGGA | 0.3 |
| CIITA | gCIITA_39 | 1310 | CTCAAAGTAGAGCACATAGGA | 0.25 |
| CIITA | gCIITA_27 | 1146 | CTCACAGCTGAGCCCCCCACT | 0.4 |
| CIITA | gCIITA_10 | 1130 | CTCACCGATATTGGCATAAGC | 0.1 |
| CIITA | gCIITA_14 | 1134 | CTCAGGCCCTCCAGCTGGGAG | 0.2 |
| CIITA | gCIITA_28 | 1147 | CTCCAGGCGCATCTGGCCGGA | 0.7 |
| CIITA | gCIITA_31 | 1149 | CTCCAGTTCCTCGTTGAGCTG | 0.1 |
| CIITA | gCIITA_35 | 1306 | CTCCCAGAACCCGACACAGAC | 48.7 |
| CIITA | gCIITA_79 | 1348 | CTCCCTGCAGCATCTGGAGTG | 1.12 |
| CIITA | gCIITA_48 | 1319 | CTCGGGAGGTCAGGGCAGGTT | 61.63 |
| CIITA | gCIITA_22 | 1141 | CTCTGCAGCCTTCCCAGAGGA | 0.6 |
| CIITA | gCIITA_15 | 1135 | CTGAAAATGTCCTTGCTCAGG | 0.2 |
| CITTA | gCIITA_21 | 1140 | CTGACTTTTCTGCCCAACTTC | 0.1 |
| CIITA | gCIITA_19 | 1138 | CTGCCCAACTTCTGCTGGCAT | 0.5 |
| CIITA | gCIITA_101 | 1369 | CTGCTGCTCCTCTCCAGCCTG | 0.23 |
| CIITA | gCIITA_66 | 1336 | CTGGGCACCCGCCTCACGCCT | 0.31 |
| CIITA | gCIITA_37 | 1308 | CTGGGCTCAGGTGCTTCCTCA | 0.45 |
| CIITA | gCIITA_74 | 1344 | CTTACGCAAACTCCAGTTTCT | 0.79 |
| CIITA | gCIITA_38 | 1309 | CTTGTCTGGGCAGCGGAACTG | 38.38 |
| CIITA | gCIITA_49 | 1320 | GAAGCTTGTTGGAGACCTCTC | 0.67 |
| CIITA | gCIITA_100 | 1368 | GCAGAGCCGGTGGAGCAGTTC | 0.46 |
| CIITA | gCIITA_65 | 1335 | GCAGCACGTGGTACAGGAGCT | 70.73 |
| CIITA | gCIITA_103 | 1370 | GCAGCCAACAGCACCTCAGCC | 0.22 |
| CIITA | gCIITA_63 | 1333 | GCCACTCAGAGCCAGCCACAG | 35.47 |
| CIITA | gCIITA_62 | 1332 | GCCATCGCCCAGGTCCTCACG | 1.29 |
| CIITA | gCIITA_104 | 1371 | GCCCAGCACAGCAATCACTCG | 0.07 |
| CIITA | gCIITA_96 | 1364 | GCTCCATCAGCCACTGACCTG | 0.29 |
| CIITA | gCIITA_95 | 1363 | GCTGGCCTGGGGCACCTCACC | 0.59 |
| CIITA | gCIITA_50 | 1321 | GGAAGCTTGTTGGAGACCTCT | 0.57 |
| CIITA | gCIITA_76 | 1346 | GGGAAAGCCTGGGGGCCTGAG | 68.93 |
| CIITA | gCIITA_1 | 1122 | GGGCTCTGACAGGTAGGACCC | 0.5 |
| CIITA | gCIITA_72 | 1342 | GGTCCCGAACAGCAGGGAGCT | 89.25 |
| CIITA | gCIITA_29 | 1041 | GTCTCTTGCAGTGCCTTTCTC | 2.4 |
| CIITA | gCIITA_2 | 1123 | TACCTTGGGGCTCTGACAGGT | 0 |
| CIITA | gCIITA_81 | 1350 | TAGGCACCCAGGTCAGTGATG | 44.56 |
| CIITA | gCIITA_4 | 986 | TAGGGGCCCCAACTCCATGGT | 13.5 |
| CIITA | gCIITA_6 | 1126 | TATGACCAGATGGACCTGGCT | 0.2 |
| CIITA | gCIITA_40 | 1311 | TCAAAGTAGAGCACATAGGAC | 15.68 |
| CIITA | gCIITA_87 | 1355 | TCCAGCCAGGTCCATCTGGTC | 0.15 |
| CIITA | gCIITA_44 | 1315 | TCCAGGCGCATCTGGCCGGAG | 39.16 |
| CIITA | gCIITA_45 | 1316 | TCCAGTTCCTCGTTGAGCTGC | 0.22 |
| CIITA | gCIITA_98 | 1366 | TCCATCTCCAGAGCACAAGAC | 0.23 |
| CIITA | gCIITA_47 | 1318 | TCCCCACCATCTCCACTCTGC | 2.05 |
| CIITA | gCIITA_7 | 1127 | TCCTCCCAGAACCCGACACAG | 0.1 |
| CIITA | gCIITA_11 | 1131 | TCCTTGTCTGGGCAGCGGAAC | 0.1 |
| CIITA | gCIITA_16 | 1136 | TCTCAAAGTAGAGCACATAGG | 0.1 |
| CIITA | gCIITA_30 | 1148 | TCTCTTGCAGTGCCTTTCTCC | 0.1 |
| CIITA | gCIITA_93 | 1361 | TCTGACTTTTCTGCCCAACTT | 0.21 |
| CIITA | gCIITA_43 | 1314 | TCTGCAGCCTTCCCAGAGGAG | 55.09 |
| CIITA | gCIITA_20 | 1139 | TCTGCCCAACTTCTGCTGGCA | 0.1 |
| CIITA | gCIITA_13 | 1133 | TCTGGGCAGCGGAACTGGACC | 0.1 |
| CIITA | gCIITA_90 | 1358 | TCTGGGCTCAGGTGCTTCCTC | 0.25 |
| CIITA | gCIITA_53 | 1324 | TCTTCTCTGTCCCCTGCCATT | 0.28 |
| CIITA | gCIITA_83 | 1352 | TCTTGCCAGCGTCCAGTACAA | 5.62 |
| CIITA | gCIITA_42 | 1313 | TGACTTTTCTGCCCAACTTCT | 2.72 |
| CIITA | gCIITA_91 | 1359 | TGCCAATATCGGTGAGGAAGC | 0.17 |
| CIITA | gCIITA_41 | 1312 | TGCCCAACTTCTGCTGGCATC | 46.21 |
| CIITA | gCIITA_60 | 1330 | TGCCGGGCAGTGTGCCAGCTC | 11.98 |
| CIITA | gCIITA_18 | 1040 | TGCTGGCATCTCCATACTCTC | 4.8 |
| CIITA | gCIITA_64 | 1334 | TGGCTGGGCTGATCTTCCAGC | 0.5 |
| CIITA | gCIITA_67 | 1337 | TGGGCACCCGCCTCACGCCTC | 12.57 |
| CIITA | gCIITA_36 | 1307 | TGGGCTCAGGTGCTTCCTCAC | 85.46 |
| CIITA | gCIITA_5 | 1125 | TTAACAGCGATGCTGACCCCC | 0.1 |
| CIITA | gCIITA_3 | 1124 | TTACCTTGGGGCTCTGACAGG | 0 |
| CIITA | gCIITA_88 | 1356 | TTCTCCAGCCAGGTCCATCTG | 0.21 |
| CIITA | gCIITA_99 | 1367 | TTGGAGACCTCTCCAGCTGCC | 0.99 |
| CIITA | gCIITA_73 | 1343 | TTTAGGTCCCGAACAGCAGGG | 10.88 |
| CTLA4 | gCTLA4_36 | 143 | ACAGCTAAAGAAAAGAAGCCC | 3.9 |
| CTLA4 | gCTLA4_40 | 147 | AGCCTTATTTTATTCCCATCA | 0.3 |
| CTLA4 | gCTLA4_4 | 116 | AGCGGCACAAGGCTCAGCTGA | 58.4 |
| CTLA4 | gCTLA4_17 | 123 | AGTCACCTGGCTGTCAGCCTG | 0.4 |
| CTLA4 | gCTLA4_20 | 126 | ATTTCCACTGGAGGTGCCCGT | 0.1 |
| CTLA4 | gCTLA4_37 | 144 | CACATAGACCCCTGTTGTAAG | 2.9 |
| CTLA4 | gCTLA4_38 | 145 | CACATTCTGGCTCTGTTGGGG | 0.2 |
| CTLA4 | gCTLA4_19 | 114 | CACTGGAGGTGCCCGTGCAGA | 42.5 |
| CTLA4 | gCTLA4_6 | 113 | CAGAAGACAGGGATGAAGAGA | 44.6 |
| CTLA4 | gCTLA4_22 | 128 | CAGATGTAGAGTCCCGTGTCC | 0.6 |
| CTLA4 | gCTLA4_29 | 135 | CAGCAGTTAGTTCGGGGTTGT | 0.7 |
| CTLA4 | gCTLA4_11 | 119 | CCATGCTAGCAATGCACGTGG | 0.1 |
| CTLA4 | gCTLA4_14 | 112 | CCTGGAGATGCATACTCACAC | 47.4 |
| CTLA4 | gCTLA4_2 | 125 | CCTTGGATTTCAGCGGCACAA | 0.8 |
| CTLA4 | gCTLA4_18 | 124 | CTAGATGATTCCATCTGCACG | 2 |
| CTLA4 | gCTLA4_23 | 129 | CTCACCAATTACATAAATCTG | 0.8 |
| CTLA4 | gCTLA4_31 | 138 | CTCCTCACAGCTGTTTCTTTG | 1 |
| CTLA4 | gCTLA4_28 | 134 | CTCCTCTGGATCCTTGCAGCA | 3 |
| CTLA4 | gCTLA4_27 | 133 | CTGTTGCAGATCCAGAACCGT | 5 |
| CTLA4 | gCTLA4_21 | 127 | GATAGTGAGGTTCACTTGATT | 0.6 |
| CTLA4 | gCTLA4_3 | 136 | GATTTCAGCGGCACAAGGCTC | 0.6 |
| CTLA4 | gCTLA4_7 | 150 | GCAGAAGACAGGGATGAAGAG | 0.2 |
| CTLA4 | gCTLA4_15 | 121 | GCCTGGAGATGCATACTCACA | 0.2 |
| CTLA4 | gCTLA4_33 | 140 | GCTCAAAGAAACAGCTGTGAG | 0.8 |
| CTLA4 | gCTLA4_24 | 130 | GCTCACCAATTACATAAATCT | 1 |
| CTLA4 | gCTLA4_9 | 152 | GCTTTTCCATGCTAGCAATGC | 0.2 |
| CTLA4 | gCTLA4_16 | 122 | GGCAGGCTGACAGCCAGGTGA | 1.2 |
| CTLA4 | gCTLA4_8 | 151 | GGCTTTTCCATGCTAGCAATG | 0.1 |
| CTLA4 | gCTLA4_12 | 120 | GTGTGTGAGTATGCATCTCCA | 0.8 |
| CTLA4 | gCTLA4_25 | 131 | GTTTTCTGTTGCAGATCCAGA | 0.1 |
| CTLA4 | gCTLA4_41 | 148 | TCAATTGATGGGAATAAAATA | 3 |
| CTLA4 | gCTLA4_39 | 146 | TCACATTCTGGCTCTGTTGGG | 0.3 |
| CTLA4 | gCTLA4_10 | 118 | TCCATGCTAGCAATGCACGTG | 0.1 |
| CTLA4 | gCTLA4_32 | 139 | TCCTCACAGCTGTTTCTTTGA | 0.7 |
| CTLA4 | gCTLA4_1 | 117 | TGCCGCTGAAATCCAAGGCAA | 1.3 |
| CTLA4 | gCTLA4_13 | 115 | TGTGTGAGTATGCATCTCCAG | 12.6 |
| CTLA4 | gCTLA4_35 | 142 | TGTGTTTGACAGCTAAAGAAA | 0.1 |
| CTLA4 | gCTLA4_5 | 149 | TTCTTCTCTTCATCCCTGTCT | 1.7 |
| CTLA4 | gCTLA4_30 | 137 | TTTATAGCTTTCTCCTCACAG | 0.6 |
| CTLA4 | gCTLA4_26 | 132 | TTTTCTGTTGCAGATCCAGAA | 0.1 |
| CTLA4 | gCTLA4_34 | 141 | TTTTTGTGTTTGACAGCTAAA | 0.5 |
| DCK | gDCK_12 | 1156 | AACAATTGTGTGAAGATTGGG | 0.8 |
| DCK | gDCK_13 | 1157 | AACATTGCACCATCTGGCAAC | 1.2 |
| DCK | gDCK_17 | 1161 | AATTTTATTTTCATACCTCAA | 0 |
| DCK | gDCK_23 | 1165 | ACCTTCCAAACATATGCCTGT | 1.2 |
| DCK | gDCK_26 | 994 | AGCTTGCCATTCAGAGAGGCA | 13.3 |
| DCK | gDCK_9 | 1042 | AGGATATTCACAAATGTTGAC | 8.1 |
| DCK | gDCK_31 | 1171 | AGGTATATTTTTGCATCTAAT | 0.05 |
| DCK | gDCK_7 | 1045 | ATCTTTCCTCACAACAGCTGC | 1.5 |
| DCK | gDCK_16 | 1160 | ATTTTCATACCTCAAATTCAT | 0.1 |
| DCK | gDCK_24 | 1166 | CAAACATATGCCTGTCTCAGT | 1.1 |
| DCK | gDCK_20 | 1164 | CAATGTCTCAGAAAAATGGTG | 0.6 |
| DCK | gDCK_15 | 1159 | CATACCTCAAATTCATCTTGA | 0.3 |
| DCK | gDCK_11 | 1155 | CCAATCTTCACACAATTGTTT | 0.1 |
| DCK | gDCK_25 | 1167 | CCATTCAGAGAGGCAAGCTGA | 0.9 |
| DCK | gDCK_5 | 1153 | CCGATGTTCCCTTCGATGGAG | 0.5 |
| DCK | gDCK_27 | 1168 | CCTCTCTGAATGGCAAGCTCA | 1.1 |
| DCK | gDCK_6 | 1433 | CGGAGGCTCCTTACCGATGTT | 85.1 |
| DCK | gDCK_8 | 993 | CTCACAACAGCTGCAGGGAAG | 31.7 |
| DCK | gDCK_3 | 1151 | CTTGATGCGGGTCCCCTCAGA | 0.3 |
| DCK | gDCK_14 | 1158 | GAACATTGCACCATCTGGCAA | 0.6 |
| DCK | gDCK_22 | 1043 | GAAGGTAAAAGACCATCGTTC | 5.6 |
| DCK | gDCK_4 | 1152 | GATGGAGATTTTCTTGATGCG | 0.3 |
| DCK | gDCK_30 | 995 | TACATACCTGTCACTATACAC | 12.8 |
| DCK | gDCK_2 | 992 | TCAGCCAGCTCTGAGGGGACC | 50.4 |
| DCK | gDCK_21 | 1044 | TCATACATCATCTGAAGAACA | 3.6 |
| DCK | gDCK_19 | 1163 | TCTGAGACATTGTAAGTTCCT | 0.7 |
| DCK | gDCK_28 | 1169 | TCTGCATCTTTGAGCTTGCCA | 0.1 |
| DCK | gDCK_1 | 1150 | TCTTGGGCGGGGTGGCCATTC | 0.1 |
| DCK | gDCK_10 | 1154 | TGAATATCCTTAAACAATTGT | 1 |
| DCK | gDCK_18 | 1162 | TGCACATTCAAAATAGGAACT | 0.4 |
| DCK | gDCK_29 | 1170 | TTGAACGATCTGTGTATAGTG | 0.2 |
| FAS | gFAS_44 | 1200 | AACAAAGCAAGAACTTACCCC | 0.3 |
| FAS | gFAS_64 | 1217 | AACTTGACTTAGTGTCATGAC | 0.4 |
| FAS | gFAS_23 | 1187 | AAGACTCTTACCATGTCCTTC | 0.6 |
| FAS | gFAS_55 | 1209 | AAGTTGGAGATTCATGAGAAC | 0.4 |
| FAS | gFAS_56 | 1210 | AATACCTACAGGATTTAAAGT | 0.3 |
| FAS | gFAS_84 | 1235 | AATTTTCTGAGTCACTAGTAA | 0.6 |
| FAS | gFAS_4 | 1058 | ACAGGTTCTTACGTCTGTTGC | 1.5 |
| FAS | gFAS_89 | 1240 | AGAAATGAAATCCAAAGCTTG | 0.5 |
| FAS | gFAS_82 | 1233 | AGGATGATAGTCTGAATTTTC | 0.4 |
| FAS | gFAS_63 | 1216 | AGTAAATATATCACCACTATT | 0.8 |
| FAS | gFAS_47 | 1046 | AGTGAAGAGAAAGGAAGTACA | 9.8 |
| FAS | gFAS_77 | 1228 | ATCAATGTGTCATACGCTTCT | 0.8 |
| FAS | gFAS_22 | 1186 | ATCACACAATCTACATCTTCT | 0.5 |
| FAS | gFAS_35 | 997 | ATGATTCCATGTTCACATCTA | 58.5 |
| FAS | gFAS_76 | 1227 | ATGGAAAGAAAGAAGCGTATG | 1.3 |
| FAS | gFAS_67 | 1220 | ATTGACACCATTCTTTCGAAC | 0.5 |
| FAS | gFAS_86 | 1237 | ATTTCTGAAGTTTGAATTTTC | 0.3 |
| FAS | gFAS_3 | 1173 | ATTTTACAGGTTCTTACGTCT | 0.7 |
| FAS | gFAS_24 | 1188 | CAAACTGATTTTCTAGGCTTA | 0.1 |
| FAS | gFAS_9 | 1177 | CAAGTTCTGAGTCTCAACTGT | 0.1 |
| FAS | gFAS_37 | 1194 | CACTTGGTGTTGCTGGTGAGT | 1.3 |
| FAS | gFAS_28 | 1191 | CATCTGCACTTGGTATTCTGG | 1.2 |
| FAS | gFAS_73 | 1224 | CATGAAGTTGATGCCAATTAC | 0.8 |
| FAS | gFAS_60 | 1213 | CCAGATAAATTTATTGCCACT | 0.7 |
| FAS | gFAS_43 | 1199 | CCCCAAACAATTAGTGGAATT | 0.4 |
| FAS | gFAS_17 | 1181 | CCTTCTTGGCAGGGCACGCAG | 0.8 |
| FAS | gFAS_53 | 1207 | CCTTTCTGTGCTTTCTGCATG | 0.3 |
| FAS | gFAS_58 | 1212 | CTAGGAAACAGTGGCAATAAA | 1.3 |
| FAS | gFAS_25 | 1048 | CTAGGCTTAGAAGTGGAAATA | 3.5 |
| FAS | gFAS_61 | 1214 | CTATTTTTCAGATGTTGACTT | 0.1 |
| FAS | gFAS_80 | 1231 | CTCTGCAAGAGTACAAAGATT | 0.2 |
| FAS | gFAS_38 | 1056 | CTCTTTGCACTTGGTGTTGCT | 1.5 |
| FAS | gFAS_83 | 1234 | CTGAGTCACTAGTAATGTCCT | 0.7 |
| FAS | gFAS_50 | 1204 | CTGCATGTTTTCTGTACTTCC | 0.4 |
| FAS | gFAS_48 | 1202 | CTGTACTTCCTTTCTCTTCAC | 0.8 |
| FAS | gFAS_52 | 1206 | CTGTGCTTTCTGCATGTTTTC | 0.3 |
| FAS | gFAS_71 | 1055 | CTGTTCTGCTGTGTCTTGGAC | 1.5 |
| FAS | gFAS_33 | 1054 | CTTGGTGCAAGGGTCACAGTG | 1.6 |
| FAS | gFAS_65 | 1218 | GAACAAAGCCTTTAACTTGAC | 0.5 |
| FAS | gFAS_20 | 1184 | GAAGAAAAATGGGCTTTGTCT | 0.7 |
| FAS | gFAS_10 | 1049 | GAAGGCCTGCATCATGATGGC | 2.4 |
| FAS | gFAS_26 | 1189 | GAAGTGGAAATAAACTGCACC | 0.3 |
| FAS | gFAS_8 | 1176 | GAGTTGATGTCAGTCACTTGG | 0.1 |
| FAS | gFAS_87 | 1238 | GATTTCATTTCTGAAGTTTGA | 0.5 |
| FAS | gFAS_42 | 1198 | GCCAATTCCACTAATTGTTTG | 0.4 |
| FAS | gFAS_5 | 1051 | GGACGATAATCTAGCAACAGA | 1.9 |
| FAS | gFAS_1 | 999 | GGAGGATTGCTCAACAACCAT | 22.6 |
| FAS | gFAS_88 | 1239 | GGATTTCATTTCTGAAGTTTG | 0.5 |
| FAS | gFAS_15 | 1059 | GGCAGGTGAAAGGAAAGCTAG | 1.5 |
| FAS | gFAS_7 | 1175 | GGCATTAACACTTTTGGACGA | 0.1 |
| FAS | gFAS_69 | 1222 | GGCTTCATTGACACCATTCTT | 0.4 |
| FAS | gFAS_39 | 1195 | GGGTGGCTTTGTCTTCTTCTT | 0.1 |
| FAS | gFAS_72 | 1223 | GTAATTGGCATCAACTTCATG | 0.3 |
| FAS | gFAS_27 | 1190 | GTATTCTGGGTCCGGGTGCAG | 1.3 |
| FAS | gFAS_92 | 1243 | GTCTAGAGTGAAAAACAACAA | 0.5 |
| FAS | gFAS_19 | 1183 | GTCTGTGTACTCCTTCCCTTC | 0.6 |
| FAS | gFAS_40 | 1196 | GTCTTCTTCTTTTGCCAATTC | 0.6 |
| FAS | gFAS_32 | 1050 | GTGCAAGGGTCACAGTGTTCA | 2.4 |
| FAS | gFAS_12 | 998 | GTGTAACATACCTGGAGGACA | 29.9 |
| FAS | gFAS_36 | 987 | GTGTTGCTGGTGAGTGTGCAT | 61.9 |
| FAS | gFAS_66 | 1219 | GTTCGAAAGAATGGTGTCAAT | 0.9 |
| FAS | gFAS_29 | 1053 | GTTTACATCTGCACTTGGTAT | 1.6 |
| FAS | gFAS_54 | 1208 | GTTTTCCTTTCTGTGCTTTCT | 0.4 |
| FAS | gFAS_81 | 1232 | TACTCTTGCAGAGAAAATTCA | 0.2 |
| FAS | gFAS_59 | 1000 | TAGGAAACAGTGGCAATAAAT | 11 |
| FAS | gFAS_2 | 1172 | TATTTTACAGGTTCTTACGTC | 0.1 |
| FAS | gFAS_90 | 1241 | TCACTCTAGACCAAGCTTTGG | 0.5 |
| FAS | gFAS_62 | 1215 | TCAGATGTTGACTTGAGTAAA | 0.6 |
| FAS | gFAS_18 | 1182 | TCTGTGTACTCCTTCCCTTCT | 1 |
| FAS | gFAS_21 | 1185 | TCTTCCAAATGCAGAAGATGT | 0.7 |
| FAS | gFAS_41 | 1197 | TCTTCTTCTTTTGCCAATTCC | 0.1 |
| FAS | gFAS_85 | 1236 | TGAAGTTTGAATTTTCTGAGT | 0.4 |
| FAS | gFAS_49 | 1203 | TGCATGTTTTCTGTACTTCCT | 0.6 |
| FAS | gFAS_6 | 1174 | TGGACGATAATCTAGCAACAG | 0 |
| FAS | gFAS_11 | 1178 | TGGCAGAATTGGCCATCATGA | 0.8 |
| FAS | gFAS_51 | 1205 | TGTGCTTTCTGCATGTTTTCT | 0.3 |
| FAS | gFAS_70 | 1057 | TGTTCTGCTGTGTCTTGGACA | 1.5 |
| FAS | gFAS_14 | 1052 | TTCCTTGGGCAGGTGAAAGGA | 1.7 |
| FAS | gFAS_68 | 1221 | TTCGAAAGAATGGTGTCAATG | 0.7 |
| FAS | gFAS_46 | 1201 | TTCTTTCAGTGAAGAGAAAGG | 0.9 |
| FAS | gFAS_78 | 1229 | TTGAGATCTTTAATCAATGTG | 1 |
| FAS | gFAS_57 | 1211 | TTGCTTTCTAGGAAACAGTGG | 1.1 |
| FAS | gFAS_16 | 1180 | TTGGCAGGGCACGCAGTCTGG | 0.7 |
| FAS | gFAS_91 | 1242 | TTGTTTTTCACTCTAGACCAA | 0.7 |
| FAS | gFAS_74 | 1225 | TTTCCATGAAGTTGATGCCAA | 0.4 |
| FAS | gFAS_13 | 1179 | TTTCCTTGGGCAGGTGAAAGG | 1.1 |
| FAS | gFAS_75 | 1226 | TTTCTTTCCATGAAGTTGATG | 0.5 |
| FAS | gFAS_79 | 1230 | TTTGAGATCTTTAATCAATGT | 0.9 |
| FAS | gFAS_31 | 1193 | TTTGTAACTCTACTGTATGTG | 1.4 |
| FAS | gFAS_45 | 1047 | TTTGTTCTTTCAGTGAAGAGA | 6 |
| FAS | gFAS_30 | 1192 | TTTTGTAACTCTACTGTATGT | 0.8 |
| FAS | gFAS_34 | 996 | TTTTTCTAGATGTGAACATGG | 59.1 |
| TIM3 | gTIM3_37 | 364 | AAAATTAAAGCGCCGAAGATA | 0.2 |
| TIM3 | gTIM3_12 | 337 | AATGTGGCAACGTGGTGCTCA | 21.9 |
| TIM3 | gTIM3_43 | 371 | AATTCTGTATCTTCTCTTTGC | 0.7 |
| TIM3 | gTIM3_23 | 351 | ACCTGAAGTTGGTCATCAAAC | 2.2 |
| TIM3 | gTIM3_27 | 355 | ACTGCAGCCTTTCCAAGGATG | 2.6 |
| TIM3 | gTIM3_21 | 349 | AGGTTAAATTTTTCATCATTC | 0.1 |
| TIM3 | gTIM3_50 | 378 | ATATACGTTCTCTTCAATGGT | 0.5 |
| TIM3 | gTIM3_13 | 340 | ATCAGTCCTGAGCACCACGTT | 1.5 |
| TIM3 | gTIM3_22 | 350 | ATGACCAACTTCAGGTTAAAT | 0.1 |
| TIM3 | gTIM3_44 | 372 | ATTTCCACAGCCTCATCTCTT | 0.4 |
| TIM3 | gTIM3_29 | 334 | CAAGGATGCTTACCACCAGGG | 59.8 |
| TIM3 | gTIM3_46 | 374 | CACAGCCTCATCTCTTTGGCC | 0.5 |
| TIM3 | gTIM3_4 | 357 | CACATCTTCCCTTTGACTGTG | 0.8 |
| TIM3 | gTIM3_35 | 362 | CATAGCAAATATCCACATTGG | 1 |
| TIM3 | gTIM3_14 | 341 | CATCAGTCCTGAGCACCACGT | 0.1 |
| TIM3 | gTIM3_38 | 365 | CATTTGAAAATTAAAGCGCCG | 0.1 |
| TIM3 | gTIM3_28 | 356 | CCAAGGATGCTTACCACCAGG | 1.9 |
| TIM3 | gTIM3_48 | 376 | CCAATCCTGAGGGAGGGAGGT | 4.5 |
| TIM3 | gTIM3_30 | 336 | CCACCAGGGGACATGGCCCAG | 22.1 |
| TIM3 | gTIM3_10 | 383 | CCCCAGCAGACGGGCACGAGG | 7.3 |
| TIM3 | gTIM3_41 | 369 | CCCCTTACTAGGGTATTCTCA | 2.2 |
| TIM3 | gTIM3_18 | 345 | CGCAAAGGAGATGTGTCCCTG | 14.4 |
| TIM3 | gTIM3_16 | 343 | CGGAAATCCCCATTTAGCCAG | 0.4 |
| TIM3 | gTIM3_36 | 363 | CGGGACTCTGGAGCAACCATC | 3.3 |
| TIM3 | gTIM3_42 | 370 | CTAGGGTATTCTCATAGCAAA | 8.5 |
| TIM3 | gTIM3_33 | 360 | CTGTTAGATTTATATCAGGGA | 1.4 |
| TIM3 | gTIM3_49 | 377 | CTTCTGAGCGAATTCCCTCTG | 0.7 |
| TIM3 | gTIM3_3 | 348 | CTTCTGCAAGCTCCATGTTTT | 0.1 |
| TIM3 | gTIM3_7 | 333 | CTTGTAAGTAGTAGCAGCAGC | 64.4 |
| TIM3 | gTIM3_26 | 354 | GAAAGGCTGCAGTGAAGTCTC | 0.1 |
| TIM3 | gTIM3_5 | 368 | GACTGTGTCCTGCTGCTGCTG | 0.8 |
| TIM3 | gTIM3_19 | 346 | GATCCGGCAGCAGTAGATCCC | 5.1 |
| TIM3 | gTIM3_47 | 375 | GCCAACCTCCCTCCCTCAGGA | 6 |
| TIM3 | gTIM3_15 | 342 | GCCAGTATCTGGATGTCCAAT | 2.9 |
| TIM3 | gTIM3_11 | 339 | GCCCCAGCAGACGGGCACGAG | 0.6 |
| TIM3 | gTIM3_17 | 344 | GCGGAAATCCCCATTTAGCCA | 0.1 |
| TIM3 | gTIM3_51 | 379 | GGGTTGTCGCTTTGCAATGCC | 0.5 |
| TIM3 | gTIM3_40 | 367 | GTTTCCCCCTTACTAGGGTAT | 1.7 |
| TIM3 | gTIM3_6 | 335 | TAAGTAGTAGCAGCAGCAGCA | 53.7 |
| TIM3 | gTIM3_9 | 382 | TACACCCCAGCCGCCCCAGGG | 1 |
| TIM3 | gTIM3_31 | 358 | TATAGCAGAGACACAGACACT | 0.3 |
| TIM3 | gTIM3_32 | 359 | TATCAGGGAGGCTCCCCAGTG | 22.4 |
| TIM3 | gTIM3_20 | 347 | TCATCATTCATTATGCCTGGG | 0.1 |
| TIM3 | gTIM3_8 | 381 | TCTCTCTATGCAGGGTCCTCA | 0.1 |
| TIM3 | gTIM3_1 | 338 | TCTTCTGCAAGCTCCATGTTT | 0.1 |
| TIM3 | gTIM3_2 | 1244 | TCTTCTGCAAGCTCCATGTTT | 0.07 |
| TIM3 | gTIM3_25 | 353 | TGACATTAGCCAAGGTCACCC | 15.7 |
| TIM3 | gTIM3_24 | 352 | TGTTGTTTCTGACATTAGCCA | 0.7 |
| TIM3 | gTIM3_34 | 361 | TGTTTCCATAGCAAATATCCA | 5.6 |
| TIM3 | gTIM3_39 | 366 | TGTTTCCCCCTTACTAGGGTA | 0.7 |
| TIM3 | gTIM3_45 | 373 | TTTCCACAGCCTCATCTCTTT | 1 |
| LAG3 | gLAG3_18 | 167 | AACGTCTCCATCATGTATAAC | 1.1 |
| LAG3 | gLAG3_44 | 192 | ACAGAGCTGTCTAGCCCAGGT | 0.4 |
| LAG3 | gLAG3_21 | 171 | ACAGTGTACGCTGGAGCAGGT | 0.1 |
| LAG3 | gLAG3_24 | 174 | ACCCTTCGACTAGAGGATGTG | 0.8 |
| LAG3 | gLAG3_3 | 180 | ACCTGGAGCCACCCAAAGCGG | 3.1 |
| LAG3 | gLAG3_12 | 161 | AGCCGCCCTGACCGCCCAGCC | 0.1 |
| LAG3 | gLAG3_10 | 159 | CACAGTGACTGCCAGCCCCCC | N.D. |
| LAG3 | gLAG3_30 | 181 | CAGTGACTCCCAAATCCTTTG | 0.1 |
| LAG3 | gLAG3_27 | 177 | CCACCTGAGGCTGACCTGTGA | 3.4 |
| LAG3 | gLAG3_41 | 156 | CCAGCCTTGGCAATGCCAGCT | 8.3 |
| LAG3 | gLAG3_28 | 178 | CCCACCTGAGGCTGACCTGTG | 0.8 |
| LAG3 | gLAG3_40 | 189 | CCCAGCCTTGGCAATGCCAGC | 0.8 |
| LAG3 | gLAG3_31 | 182 | CCCAGGGATCCAGGTGACCCA | 3.1 |
| LAG3 | gLAG3_25 | 175 | CCCTTCGACTAGAGGATGTGA | 2.7 |
| LAG3 | gLAG3_7 | 206 | CCGCCCAGTGGCCCGCCCGCT | N.D. |
| LAG3 | gLAG3_14 | 163 | CCGCTAAGTGGTGATGGGGGG | 0.3 |
| LAG3 | gLAG3_13 | 162 | CGCTAAGTGGTGATGGGGGGA | 2.3 |
| LAG3 | gLAG3_23 | 173 | CTCACTGCCAAGTGGACTCCT | 0.4 |
| LAG3 | gLAG3_45 | 193 | CTCCATAGGTGCCCAACGCTC | 1.3 |
| LAG3 | gLAG3_55 | 203 | CTCTAAGGCAGAAAATCGTCT | 0.1 |
| LAG3 | gLAG3_49 | 197 | CTCTGCTCCTTTTGGTGACTG | 0.2 |
| LAG3 | gLAG3_20 | 170 | CTCTTCAGGTCTGGAGCCCCC | 0.2 |
| LAG3 | gLAG3_17 | 166 | CTCTTCCTGCCCCAAGTCAGC | 1.3 |
| LAG3 | gLAG3_56 | 204 | CTGCCTTAGAGCAAGGGATTC | 0.1 |
| LAG3 | gLAG3_1 | 158 | CTGTTTCTGCAGCCGCTTTGG | 0.2 |
| LAG3 | gLAG3_19 | 168 | CTTTTCTCTTCAGGTCTGGAG | 0.2 |
| LAG3 | gLAG3_11 | 160 | GAACTGCTCCTTCAGCCGCCC | 0.1 |
| LAG3 | gLAG3_26 | 176 | GACTAGAGGATGTGAGCCAGG | 1 |
| LAG3 | gLAG3_57 | 205 | GAGCAAGGGATTCACCCTCCG | 0.2 |
| LAG3 | gLAG3_42 | 190 | GCAATGCCAGCTGTACCAGGG | 0.6 |
| LAG3 | gLAG3_22 | 172 | GCAGTGAGGAAAGACCGGGTC | 2.1 |
| LAG3 | gLAG3_15 | 164 | GCGGAAAGCTTCCTCTTCCTG | 1 |
| LAG3 | gLAG3_4 | 188 | GCTCACCTAGTGAAGCCTCTC | 1.3 |
| LAG3 | gLAG3_39 | 187 | GCTGGAGGCACAGGAGGCCCA | 0.3 |
| LAG3 | gLAG3_54 | 202 | GCTTTCACCTTTGGAGAAGAC | 0.2 |
| LAG3 | gLAG3_53 | 201 | GGCTTTCACCTTTGGAGAAGA | 0.1 |
| LAG3 | gLAG3_16 | 165 | GGGCAGGAAGAGGAAGCTTTC | 6.4 |
| LAG3 | gLAG3_32 | 183 | GGGTCACCTGGATCCCTGGGG | 0.2 |
| LAG3 | gLAG3_6 | 153 | GGGTGCATACCTGTCTGGCTG | 52.4 |
| LAG3 | gLAG3_33 | 154 | GGTCACCTGGATCCCTGGGGA | 17.1 |
| LAG3 | gLAG3_52 | 200 | GGTGACTGGAGCCTTTGGCTT | 0.2 |
| LAG3 | gLAG3_34 | 184 | GTGAGGTGACTCCAGTATCTG | 0.7 |
| LAG3 | gLAG3_48 | 196 | GTGTCCTTTCTCTGCTCCTTT | 0.1 |
| LAG3 | gLAG3_36 | 185 | GTGTGGAGCTCTCTGGACACC | 0.9 |
| LAG3 | gLAG3_29 | 179 | TACTCTTTTCAGTGACTCCCA | 0.3 |
| LAG3 | gLAG3_38 | 155 | TCAGGACCTTGGCTGGAGGCA | 17.7 |
| LAG3 | gLAG3_47 | 195 | TCATCCTTGGTGTCCTTTCTC | 0.4 |
| LAG3 | gLAG3_46 | 194 | TCCATAGGTGCCCAACGCTCT | 4 |
| LAG3 | gLAG3_9 | 208 | TCCTTGCACAGTGACTGCCAG | N.D. |
| LAG3 | gLAG3_8 | 207 | TCGCTATGGCTGCGCCCAGCC | 0.1 |
| LAG3 | gLAG3_50 | 1245 | TCTGCTCCTTTTGGTGACTGG | 0.1 |
| LAG3 | gLAG3_35 | 157 | TGAGGTGACTCCAGTATCTGG | 9.3 |
| LAG3 | gLAG3_2 | 169 | TGCAGCCGCTTTGGGTGGCTC | 0.2 |
| LAG3 | gLAG3_5 | 198 | TGCGAAGAGCAGGGGTCACTT | 0.8 |
| LAG3 | gLAG3_51 | 199 | TGGTGACTGGAGCCTTTGGCT | 0.6 |
| LAG3 | gLAG3_37 | 186 | TGTGGAGCTCTCTGGACACCC | 6.9 |
| LAG3 | gLAG3_43 | 191 | TTGGAGCAGCAGTGTACTTCA | 0.8 |
| PD | gPD_1 | 214 | AACCTGACCTGGGACAGTTTC | 0.2 |
| PD | gPD_7 | 237 | ACCTGCAGCTTCTCCAACACA | 0.2 |
| PD | gPD_16 | 220 | ATCTGCGCCTTGGGGGCCAGG | 1.2 |
| PD | gPD_14 | 218 | CACATGAGCGTGGTCAGGGCC | 0.1 |
| PD | gPD_20 | 225 | CAGAGAGAAGGGCAGAAGTGC | 2.5 |
| PD | gPD_27 | 211 | CAGTGGCGAGAGAAGACCCCG | 23.7 |
| PD | gPD_12 | 216 | CCCGAGGACCGCAGCCAGCCC | 0.4 |
| PD | gPD_28 | 231 | CCTAGCGGAATGGGCACCTCA | 0.1 |
| PD | gPD_2 | 224 | CCTTCCGCTCACCTCCGCCTG | 46.9 |
| PD | gPD_3 | 232 | CGCTCACCTCCGCCTGAGCAG | 1 |
| PD | gPD_13 | 217 | CGTGTCACACAACTGCCCAAC | 0.5 |
| PD | gPD_29 | 212 | CTAGCGGAATGGGCACCTCAT | 30.3 |
| PD | gPD_24 | 228 | CTCCTCAAAGAAGGAGGACCC | 0.1 |
| PD | gPD_22 | 227 | GAACTGGCCGGCTGGCCTGGG | 1.7 |
| PD | gPD_15 | 219 | GATCTGCGCCTTGGGGGCCAG | 0.1 |
| PD | gPD_8 | 209 | GCACGAAGCTCTCCGATGTGT | 41.7 |
| PD | gPD_30 | 233 | GCCCCTCTGACCGGCTTCCTT | 0.3 |
| PD | gPD_17 | 221 | GGGGCCAGGGAGATGGCCCCA | 0.6 |
| PD | gPD_6 | 236 | GGTCACCACGAGCAGGGCTGG | 0.7 |
| PD | gPD_18 | 222 | GTGCCCTTCCAGAGAGAAGGG | 1.7 |
| PD | gPD_10 | 213 | GTGCTAAACTGGTACCGCATG | 0.2 |
| PD | gPD_9 | 238 | TCCAACACATCGGAGAGCTTC | 0.2 |
| PD | gPD_4 | 234 | TCCACTGCTCAGGCGGAGGTG | 0.6 |
| PD | gPD_5 | 235 | TCCCCAGCCCTGCTCGTGGTG | 1.2 |
| PD | gPD_11 | 215 | TCCGTCTGGTTGCTGGGGCTC | 0.1 |
| PD | gPD_25 | 229 | TCCTCAAAGAAGGAGGACCCC | 0.5 |
| PD | gPD_26 | 230 | TCTCGCCACTGGAAATCCAGC | 0.2 |
| PD | gPD_23 | 210 | TCTGCAGGGACAATAGGAGCC | 57.6 |
| PD | gPD_19 | 223 | TGCCCTTCCAGAGAGAAGGGC | 0.9 |
| PD | gPD_21 | 226 | TGCCCTTCTCTCTGGAAGGGC | 1.4 |
| PTPN6 | gPTPN6_22 | 268 | AAGAAGACGGGGATTGAGGAG | 22.3 |
| PTPN6 | gPTPN6_48 | 291 | AATGAACTGGGCGATGGCCAC | 3.3 |
| PTPN6 | gPTPN6_1 | 254 | ACCGAGACCTCAGTGGGCTGG | 58.2 |
| PTPN6 | gPTPN6_46 | 252 | ACTGCCCCCCACCCAGGCCTG | 80.3 |
| PTPN6 | gPTPN6_2 | 265 | AGCAGGGTCTCTGCATCCAGC | 0.3 |
| PTPN6 | gPTPN6_8 | 300 | AGGTGGATGATGGTGCCGTCG | 3.5 |
| PTPN6 | gPTPN6_30 | 275 | ATGTAGTTGGCATTGATGTAG | 0.2 |
| PTPN6 | gPTPN6_17 | 262 | ATGTGGGTGACCCTGAGCGGG | 0.9 |
| PTPN6 | gPTPN6_28 | 273 | CACCAGCGTCTGGAAGGGCAG | 5.4 |
| PTPN6 | gPTPN6_36 | 281 | CAGAACAAATGCGTCCCATAC | 0.5 |
| PTPN6 | gPTPN6_26 | 251 | CAGAAGCAGGAGGTGAAGAAC | 77.5 |
| PTPN6 | gPTPN6_27 | 272 | CAGACGCTGGTGCAAGTTCTT | 0.3 |
| PTPN6 | gPTPN6_39 | 283 | CAGGTCTCCCCGCTGGACAAT | 1.6 |
| PTPN6 | gPTPN6_35 | 280 | CCAGAACAAATGCGTCCCATA | 0.2 |
| PTPN6 | gPTPN6_25 | 271 | CCCACCCACATCTCAGAGTTT | 34.8 |
| PTPN6 | gPTPN6_44 | 1246 | CCCAGCGCCGGCATCGGCCGC | N.D. |
| PTPN6 | gPTPN6_53 | 295 | CCCCCCTGCACCCGGCTGCAG | 7 |
| PTPN6 | gPTPN6_18 | 263 | CCTCGCACATGACCTTGATGT | 1.4 |
| PTPN6 | gPTPN6_9 | 301 | CCTGACGCTGCCTTCTCTAGG | 0.8 |
| PTPN6 | gPTPN6_43 | 288 | CCTGCCGCTGGTTGATCTGGT | 0.3 |
| PTPN6 | gPTPN6_7 | 250 | CGACTCTGACAGAGCTGGTGG | 78.1 |
| PTPN6 | gPTPN6_31 | 276 | CGTCCAGAACCAGCTGCTAGG | 0.3 |
| PTPN6 | gPTPN6_34 | 279 | CTCCACCTCTCGGGTGGTCAT | 1.2 |
| PTPN6 | gPTPN6_56 | 298 | CTCCTCCCTCTTGTTCTTAGT | 0.1 |
| PTPN6 | gPTPN6_42 | 287 | CTGCCGCTGGTTGATCTGGTC | 5.3 |
| PTPN6 | gPTPN6_41 | 286 | CTGGACCAGATCAACCAGCGG | 8.4 |
| PTPN6 | gPTPN6_4 | 284 | CTGGCTCGGCCCAGTCGCAAG | 4.3 |
| PTPN6 | gPTPN6_15 | 260 | CTGTGCTCAGTGACCAGCCCA | 0.5 |
| PTPN6 | gPTPN6_21 | 267 | GACAGCCTCACGGACCTGGTG | 0.5 |
| PTPN6 | gPTPN6_51 | 1248 | GACGAGGTGCGGGAGGCCTTG | N.D. |
| PTPN6 | gPTPN6_20 | 266 | GAGACCTTCGACAGCCTCACG | 9.7 |
| PTPN6 | gPTPN6_52 | 294 | GAGTCTAGTGCAGGGACCGTG | 0.1 |
| PTPN6 | gPTPN6_50 | 1247 | GCATGGGCATTCTTCATGGCT | N.D. |
| PTPN6 | gPTPN6_11 | 256 | GCCTGCAGCAGCGTCTCTGCC | 0.2 |
| PTPN6 | gPTPN6_19 | 264 | GCTCCCCCCAGGGTGGACGCT | 13.5 |
| PTPN6 | gPTPN6_24 | 270 | GCTGTATCCTCGGACTCCTGC | 0.4 |
| PTPN6 | gPTPN6_14 | 259 | GGCTGGTCACTGAGCACAGAA | 10.4 |
| PTPN6 | gPTPN6_40 | 285 | GGGAGACCTGATTCGGGAGAT | 3.4 |
| PTPN6 | gPTPN6_13 | 258 | GTGCTTTCTGTGCTCAGTGAC | 0.8 |
| PTPN6 | gPTPN6_45 | 289 | GTGGAGATGTTCTCCATGAGC | N.D. |
| PTPN6 | gPTPN6_47 | 290 | TACTGCGCCTCCGTCTGCACC | 0.1 |
| PTPN6 | gPTPN6_6 | 249 | TATGACCTGTATGGAGGGGAG | 83.4 |
| PTPN6 | gPTPN6_38 | 282 | TATTCGGTTGTGTCATGCTCC | 0.1 |
| PTPN6 | gPTPN6_33 | 278 | TCCACCTCTCGGGTGGTCATG | 0.7 |
| PTPN6 | gPTPN6_5 | 293 | TCCCCTCCATACAGGTCATAG | 14.8 |
| PTPN6 | gPTPN6_55 | 297 | TCCTCCCTCTTGTTCTTAGTG | 0 |
| PTPN6 | gPTPN6_10 | 255 | TCTAGGTGGTACCATGGCCAC | 2.4 |
| PTPN6 | gPTPN6_32 | 277 | TGGCAGATGGCGTGGCAGGAG | 4.4 |
| PTPN6 | gPTPN6_37 | 253 | TGGGCCCTACTCTGTGACCAA | 51.3 |
| PTPN6 | gPTPN6_54 | 296 | TGTCTGCAGCCGGGTGCAGGG | 0.9 |
| PTPN6 | gPTPN6_16 | 261 | TGTGCTCAGTGACCAGCCCAA | 37.5 |
| PTPN6 | gPTPN6_57 | 299 | TTCACTTTCTCCTCCCTCTTG | 0.2 |
| PTPN6 | gPTPN6_29 | 274 | TTCTCTGGCCGCTGCCCTTCC | 0.1 |
| PTPN6 | gPTPN6_49 | 292 | TTCTTAGTGGTTTCAATGAAC | 0.1 |
| PTPN6 | gPTPN6_12 | 257 | TTGTGCGTGAGAGCCTCAGCC | 29.4 |
| PTPN6 | gPTPN6_23 | 269 | TTGTTCAGTTCCAACACTCGG | 0.1 |
| TIGIT | gTIGIT_13 | 309 | AAGGATCGAGTGGCCCCAGGT | 0.2 |
| TIGIT | gTIGIT_12 | 308 | AAGGATGGGGAGATGTGCCAC | 0.4 |
| TIGIT | gTIGIT_31 | 327 | AATGTCCTGAGTTACAGAAGC | 0.5 |
| TIGIT | gTIGIT_2 | 302 | AGGCCTTACCTGAGGCGAGGG | 81.7 |
| TIGIT | gTIGIT_26 | 321 | CACAGAATGGATTCTGAGGGC | 0.3 |
| TIGIT | gTIGIT_1 | 307 | CCTGAGGCGAGGGGAGCCTGC | 0.2 |
| TIGIT | gTIGIT_16 | 312 | CTAGGACCTCCAGGAAGATTC | 0.5 |
| TIGIT | gTIGIT_21 | 316 | CTAGTCAACGCGACCACCACG | 0.1 |
| TIGIT | gTIGIT_17 | 313 | CTCCAGCAGGAATACCTGAGC | 0.8 |
| TIGIT | gTIGIT_27 | 322 | CTCCTGAGGTCACCTTCCACA | 1.6 |
| TIGIT | gTIGIT_6 | 330 | CTCTGCAGAAATGTTCCCCGT | 0.1 |
| TIGIT | gTIGIT_28 | 323 | CTGGGGGTGAGGGAGCACTGG | 0.5 |
| TIGIT | gTIGIT_19 | 314 | GAGCCATGGCCGCGACGCTGG | 0.9 |
| TIGIT | gTIGIT_11 | 304 | GGGTGGCACATCTCCCCATCC | 9.7 |
| TIGIT | gTIGIT_18 | 303 | GTCCTCCCTCTAGTGGCTGAG | 72.4 |
| TIGIT | gTIGIT_3 | 325 | GTCCTCTTCCCTAGGAATGAT | 1.3 |
| TIGIT | gTIGIT_10 | 306 | TAATGCTGACTTGGGGTGGCA | 1.6 |
| TIGIT | gTIGIT_25 | 320 | TAGAAGAAAGCCCTCAGAATC | 1.2 |
| TIGIT | gTIGIT_15 | 311 | TAGGACCTCCAGGAAGATTCT | 0.4 |
| TIGIT | gTIGIT_20 | 315 | TAGTCAACGCGACCACCACGA | 0.1 |
| TIGIT | gTIGIT_22 | 317 | TAGTTTGTTTGTTTTTAGAAG | 0.6 |
| TIGIT | gTIGIT_4 | 328 | TATTGTGCCTGTCATCATTCC | 1 |
| TIGIT | gTIGIT_5 | 329 | TCTGCAGAAATGTTCCCCGTT | 1.1 |
| TIGIT | gTIGIT_7 | 305 | TGCAGAGAAAGGTGGCTCTAT | 6 |
| TIGIT | gTIGIT_14 | 310 | TGCATCTATCACACCTACCCT | 1.4 |
| TIGIT | gTIGIT_8 | 331 | TGCCGTGGTGGAGGAGAGGTG | 0.3 |
| TIGIT | gTIGIT_29 | 324 | TGCCTGGACACAGCTTCCTGG | 0.3 |
| TIGIT | gTIGIT_9 | 332 | TGGCCATTTGTAATGCTGACT | 0.8 |
| TIGIT | gTIGIT_30 | 326 | TGTAACTCAGGACATTGAAGT | 0.5 |
| TIGIT | gTIGIT_23 | 318 | TTTGTTTTTAGAAGAAAGCCC | 1 |
| TIGIT | gTIGIT_24 | 319 | TTTTTAGAAGAAAGCCCTCAG | 0.4 |
| TRAC | gTRAC019 | 1070 | AACTATAAATCAGAACACCTG | 4.5 |
| TRAC | gTRAC089 | 1283 | AACTCAGGGTTGAGAAAACAG | 0.7 |
| TRAC | gTRAC034 | 1265 | AAGAAGATCCTATTAAATAAA | 0.1 |
| TRAC | gTRAC080 | 1278 | AATTCCTCCACTTCAACACCT | 0.5 |
| TRAC | gTRAC015 | 1256 | ACCTGCAAAATGAATATGGTG | 0 |
| TRAC | gTRAC065 | 1274 | ACTAAGAAACAGTGAGCCTTG | 0.2 |
| TRAC | gTRAC090 | 1284 | ACTCAGGGTTGAGAAAACAGC | 0.1 |
| TRAC | gTRAC044 | 1063 | AGAATCAAAATCGGTGAATAG | 7.4 |
| TRAC | gTRAC060 | 1272 | AGACATCATTGACCAGAGCTC | 1.3 |
| TRAC | gTRAC035 | 1062 | AGGTTTCCTTGAGTGGCAGGC | 7.5 |
| TRAC | gTRAC037 | 1266 | AGTGAACGTTCACGGCCAGGC | 0.7 |
| TRAC | gTRAC007 | 1081 | ATAAACTGTAAAGTACCAAAC | 1.7 |
| TRAC | gTRAC030 | 1077 | ATAGGATCTTCTTCAAAACCC | 2.2 |
| TRAC | gTRAC079 | 1006 | ATTCCTCCACTTCAACACCTG | 45.4 |
| TRAC | gTRAC048 | 1071 | ATTCTCAAACAAATGTGTCAC | 4.5 |
| TRAC | gTRAC032 | 1264 | ATTTAATAGGATCTTCTTCAA | 0.1 |
| TRAC | gTRAC055 | 1270 | CACATGCAAAGTCAGATTTGT | 1 |
| TRAC | gTRAC017 | 1434 | CAGGTGAAATTCCTGAGATGT | 63.6 |
| TRAC | gTRAC010 | 1254 | CAGTTTATTAAATAGATGTTT | 0.5 |
| TRAC | gTRAC056 | 1073 | CATGTGCAAACGCCTTCAACA | 3.9 |
| TRAC | gTRAC023 | 1259 | CCAACTTAATGCCAACATACC | 1.4 |
| TRAC | gTRAC078 | 1002 | CCAGCTCACTAAGTCAGTCTC | 47.4 |
| TRAC | gTRAC082 | 1016 | CCAGCTGACAGATGGGCTCCC | 21.5 |
| TRAC | gTRAC028 | 1022 | CCATGCCTGCCTTTACTCTGC | 15.3 |
| TRAC | gTRAC083 | 1083 | CCCAGCTGACAGATGGGCTCC | 1.6 |
| TRAC | gTRAC027 | 1262 | CCCATGCCTGCCTTTACTCTG | 0.7 |
| TRAC | gTRAC041 | 1018 | CCCCAACCCAGGCTGGAGTCC | 18.7 |
| TRAC | gTRAC072 | 1064 | CCCCTTACTGCTCTTCTAGGC | 6.9 |
| TRAC | gTRAC068 | 1068 | CCCGTGTCATTCTCTGGACTG | 5.3 |
| TRAC | gTRAC040 | 1017 | CCGTATAAAGCATGAGACCGT | 21.5 |
| TRAC | gTRAC067 | 1005 | CCGTGTCATTCTCTGGACTGC | 45.4 |
| TRAC | gTRAC042 | 1061 | CCTCTTTGCCCCAACCCAGGC | 7.6 |
| TRAC | gTRAC005 | 1252 | CCTTAGTGCTGAGACTCATTC | 0.6 |
| TRAC | gTRAC003 | 1250 | CGTAGGATTTTGTGTTTTTAA | 0.1 |
| TRAC | gTRAC066 | 1060 | CTAAGAAACAGTGAGCCTTGT | 9.5 |
| TRAC | gTRAC086 | 1280 | CTCAACCCTGAGTTAAAACAC | 0.2 |
| TRAC | gTRAC071 | 1075 | CTCAGACTGTTTGCCCCTTAC | 3.4 |
| TRAC | gTRAC018 | 1013 | CTCGATATAAGGCCTTGAGCA | 26 |
| TRAC | gTRAC029 | 1021 | CTCTGCCAGAGTTATATTGCT | 15.8 |
| TRAC | gTRAC025 | 1069 | CTGGGCCTTTTTCCCATGCCT | 4.6 |
| TRAC | gTRAC004 | 1251 | CTTAGTGCTGAGACTCATTCT | 0.7 |
| TRAC | gTRAC036 | 1072 | CTTGAGTGGCAGGCCAGGCCT | 4.4 |
| TRAC | gTRAC058 | 1009 | CTTGCTTCAGGAATGGCCAGG | 27.8 |
| TRAC | gTRAC024 | 1260 | CTTTGCTGGGCCTTTTTCCCA | 1 |
| TRAC | gTRAC020 | 1066 | GAACTATAAATCAGAACACCT | 6.4 |
| TRAC | gTRAC033 | 1078 | GAAGAAGATCCTATTAAATAA | 2 |
| TRAC | gTRAC059 | 1435 | GACATCATTGACCAGAGCTCT | 50.1 |
| TRAC | gTRAC084 | 1082 | GACTTTTCCCAGCTGACAGAT | 1.6 |
| TRAC | gTRAC043 | 1014 | GAGTCTCTCAGCTGGTACACG | 25.9 |
| TRAC | gTRAC047 | 1269 | GATTCTCAAACAAATGTGTCA | 0.1 |
| TRAC | gTRAC073 | 1001 | GCAGACAGGGAGAAATAAGGA | 66.9 |
| TRAC | gTRAC016 | 1257 | GCAGGTGAAATTCCTGAGATG | 0.2 |
| TRAC | gTRAC074 | 1012 | GGCAGACAGGGAGAAATAAGG | 27.1 |
| TRAC | gTRAC062 | 1065 | GGTGGCAATGGATAAGGCCGA | 6.5 |
| TRAC | gTRAC009 | 1080 | GTACTTTACAGTTTATTAAAT | 1.7 |
| TRAC | gTRAC088 | 1282 | GTCCTGAAGGTAGCTGTTTTC | 0.1 |
| TRAC | gTRAC050 | 1023 | GTCTGTGATATACACATCAGA | 11.4 |
| TRAC | gTRAC057 | 1271 | GTGCCTTCGCAGGCTGTTTCC | 0.9 |
| TRAC | gTRAC061 | 1008 | GTGGCAATGGATAAGGCCGAG | 38.8 |
| TRAC | gTRAC002 | 1249 | GTGTTTTTAATGTGACTCTCA | 0.4 |
| TRAC | gTRAC039 | 1004 | TAAGATGCTATTTCCCGTATA | 45.8 |
| TRAC | gTRAC081 | 1076 | TAATTCCTCCACTTCAACACC | 2.3 |
| TRAC | gTRAC038 | 1007 | TACGGGAAATAGCATCTTAGA | 40.7 |
| TRAC | gTRAC064 | 1074 | TACTAAGAAACAGTGAGCCTT | 3.5 |
| TRAC | gTRAC021 | 1010 | TAGTTCAAAACCTCTATCAAT | 27.7 |
| TRAC | gTRAC012 | 1003 | TATGGAGAAGCTCTCATTTCT | 46.7 |
| TRAC | gTRAC085 | 1279 | TCAACCCTGAGTTAAAACACA | 0.5 |
| TRAC | gTRAC014 | 1020 | TCAGAAGAGCCTGGCTAGGAA | 16.6 |
| TRAC | gTRAC026 | 1261 | TCCCATGCCTGCCTTTACTCT | 0.6 |
| TRAC | gTRAC069 | 1275 | TCCCGTGTCATTCTCTGGACT | 1 |
| TRAC | gTRAC077 | 1277 | TCCCTGTCTGCCAAAAAATCT | 1.1 |
| TRAC | gTRAC087 | 1281 | TCCTGAAGGTAGCTGTTTTCT | 0.2 |
| TRAC | gTRAC049 | 1011 | TCTGTGATATACACATCAGAA | 27.6 |
| TRAC | gTRAC046 | 1268 | TGACACATTTGTTTGAGAATC | 0.2 |
| TRAC | gTRAC006 | 988 | TGAGGGTGAAGGATAGACGCT | 81.8 |
| TRAC | gTRAC075 | 1015 | TGGCAGACAGGGAGAAATAAG | 25.2 |
| TRAC | gTRAC022 | 1258 | TGGTATGTTGGCATTAAGTTG | 1 |
| TRAC | gTRAC001 | 1079 | TGTTTTTAATGTGACTCTCAT | 1.8 |
| TRAC | gTRAC011 | 1255 | TTAAATAGATGTTTATATGGA | 0 |
| TRAC | gTRAC063 | 1273 | TTAGTAAAAAGAGGGTTTTGG | 1.4 |
| TRAC | gTRAC070 | 1276 | TTCCCGTGTCATTCTCTGGAC | 0.3 |
| TRAC | gTRAC076 | 1019 | TTGGCAGACAGGGAGAAATAA | 16.7 |
| TRAC | gTRAC031 | 1263 | TTTAATAGGATCTTCTTCAAA | 0.3 |
| TRAC | gTRAC013 | 1067 | TTTCTCAGAAGAGCCTGGCTA | 5.8 |
| TRAC | gTRAC045 | 1267 | TTTGAGAATCAAAATCGGTGA | 1.3 |
| TRAC | gTRAC008 | 1253 | TTTGGTACTTTACAGTTTATT | 0.2 |
| TRBC1 + 2 | gTRBC1 + 2_1 | 1372 | AGCCATCAGAAGCAGAGATCT | 66.40 |
| | | | | (TRBC1); |
| | | | | 74.7 |
| | | | | (TRBC2) |
| TRBC1 + 2 | gTRBC1 + 2_3 | 1373 | CGCTGTCAAGTCCAGTTCTAC | 71.28 |
| | | | | (TRBC1) |
| TRBC2 | gTRBC2_11 | 1378 | AGACTGTGGCTTCACCTCCGG | 19.97 |
| TRBC2 | gTRBC2_19 | 1386 | CACAGGTCAAGAGAAAGGATT | 1.58 |
| TRBC2 | gTRBC2_10 | 1377 | CAGACTGTGGCTTCACCTCCG | 0.16 |
| TRBC2 | gTRBC2_14 | 1381 | CCAGCAAGGGGTCCTGTCTGC | 6.69 |
| TRBC2 | gTRBC2_17 | 1384 | CCATGGCCATCAGCACGAGGG | 1.75 |
| TRBC2 | gTRBC2_7 | 1374 | CCCTGTTTTCTTTCAGACTGT | 0.09 |
| TRBC2 | gTRBC2_12 | 1379 | CCGGAGGTGAAGCCACAGTCT | 33.14 |
| TRBC2 | gTRBC2_18 | 1385 | CCTAGCAAGATCTCATAGAGG | 0.37 |
| TRBC2 | gTRBC2_15 | 1382 | CTAGGGAAGGCCACCTTGTAT | 21.74 |
| TRBC2 | gTRBC2_8 | 1375 | CTTTCAGACTGTGGCTTCACC | 0.24 |
| TRBC2 | gTRBC2_21 | 1387 | GAGCTAGCCTCTGGAATCCTT | 11.89 |
| TRBC2 | gTRBC2_16 | 1383 | TATGCCGTGCTGGTCAGTGCC | 0.2 |
| TRBC2 | gTRBC2_13 | 1380 | TCAACAGAGTCTTACCAGCAA | 1.2 |
| TRBC2 | gTRBC2_9 | 1376 | TTTCAGACTGTGGCTTCACCT | 0.24 |
| CARD11 | gCARD11_2 | 1389 | ATCTTGTAGTACCGCTCCTGG | 0.07 |
| CARD11 | gCARD11_3 | 1390 | CTTCATCTTGTAGTACCGCTC | 0.08 |
| CARD11 | gCARD11_1 | 1388 | TAGTACCGCTCCTGGAAGGTT | 1.37 |
| CD247 | gCD247_23 | 104 | ACGCCAGGGTCTCAGTACAGC | 0.3 |
| CD247 | gCD247_19 | 99 | ACTCCCAAACAACCAGCGCCG | 43.17 |
| CD247 | gCD247_15 | 95 | ATCCCAATCTCACTGTAGGCC | 31.12 |
| CD247 | gCD247_16 | 96 | CATCCCAATCTCACTGTAGGC | 0.1 |
| CD247 | gCD247_7 | 109 | CCCCCATCTCAGGGTCCCGGC | 6.43 |
| CD247 | gCD247_11 | 92 | CCGTTGTCTTTCCTAGCAGAG | 1.18 |
| CD247 | gCD247_3 | 105 | CGGAGGGTCTACGGCGAGGCT | 20.79 |
| CD247 | gCD247_2 | 100 | CGTTATAGAGCTGGTTCTGGC | 0.2 |
| CD247 | gCD247_12 | 89 | CTAGCAGAGAAGGAAGAACCC | 70.64 |
| CD247 | gCD247_17 | 97 | CTCATTTCACTCCCAAACAAC | 0.3 |
| CD247 | gCD247_10 | 91 | CTGAGGGTTCTTCCTTCTCTG | 0.05 |
| CD247 | gCD247_22 | 103 | CTTTCACGCCAGGGTCTCAGT | 8.24 |
| CD247 | gCD247_8 | 110 | GACAAGAGACGTGGCCGGGAC | 40.95 |
| CD247 | gCD247_18 | 98 | TCATTTCACTCCCAAACAACC | 44.34 |
| CD247 | gCD247_6 | 108 | TCCAAAACATCGTACTCCTCT | 0.34 |
| CD247 | gCD247_9 | 111 | TCTCCCTCTAACGTCTTCCCG | 4.13 |
| CD247 | gCD247_5 | 107 | TCTGTTATAGGAGCTCAATCT | 0.24 |
| CD247 | gCD247_21 | 102 | TGATTTGCTTTCACGCCAGGG | 5.23 |
| CD247 | gCD247_14 | 94 | TGCAGGAACTGCAGAAAGATA | 2.91 |
| CD247 | gCD247_13 | 93 | TGCAGTTCCTGCAGAAGAGGG | 4.93 |
| CD247 | gCD247_1 | 90 | TGTGTTGCAGTTCAGCAGGAG | 55.77 |
| CD247 | gCD247_4 | 106 | TTATCTGTTATAGGAGCTCAA | 12.31 |
| CD247 | gCD247_20 | 101 | TTTTCTGATTTGCTTTCACGC | 0.1 |
| IL7R | gIL7R_6 | 1396 | AGTTTTTTCTCTGTCGCTCTG | 0.06 |
| IL7R | gIL7R_3 | 1393 | CAGGGGAGATGGATCCTATCT | 87.87 |
| IL7R | gIL7R_8 | 1398 | CATAACACACAGGCCAAGATG | 25.83 |
| IL7R | gIL7R_2 | 1392 | CCAGGGGAGATGGATCCTATC | 8.35 |
| IL7R | gIL7R_4 | 1394 | CTAACCATCAGCATTTTGAGT | 0.11 |
| IL7R | gIL7R_1 | 1391 | CTTTCCAGGGGAGATGGATCC | 0.25 |
| IL7R | gIL7R_5 | 1395 | GAGTTTTTTCTCTGTCGCTCT | 0.07 |
| IL7R | gIL7R_7 | 1397 | TCTGTCGCTCTGTTGGTCATC | 2.61 |
| LCK1 | gLCK1_3 | 1401 | ACCCATCAACCCGTAGGGATG | 16.21 |
| LCK1 | gLCK1_1 | 1399 | ATGTCCTTTCACCCATCAACC | 0.06 |
| LCK1 | gLCK1_2 | 1400 | CACCCATCAACCCGTAGGGAT | 0.17 |
| PLCG1 | gPLCG1_2 | 1403 | CCTTTCTGCGCTTCGTGGTGT | 5.14 |
| PLCG1 | gPLCG1_1 | 1402 | CTCATACACCACGAAGCGCAG | 0.09 |
| PLCG1 | gPLCG1_3 | 1404 | CTGCGCTTCGTGGTGTATGAG | 0.05 |
| PLCG1 | gPLCG1_5 | 1406 | GTGGTGTATGAGGAAGACATG | 3.53 |
| PLCG1 | gPLCG1_4 | 1405 | TGCGCTTCGTGGTGTATGAGG | 1.91 |
| DHODH | gDHODH_3 | 1416 | TATGCTGAACACCTGATGCCG | 74.94 |
| DHODH | gDHODH_1 | 1414 | TTGCAGAAGCGGGCCCAGGAT | 0.6 |
| DHODH | gDHODH_2 | 1415 | TTGCAGAAGCGGGCCCAGGAT | 0.59 |
| MVD | gMVD_1 | 1427 | CAGTTAAAAACCACCACAACA | 1.42 |
| MVD | gMVD_2 | 1428 | GCTGAATGGCCGGGAGGAGGA | 14.06 |
| MVD | gMVD_3 | 1429 | TGGAGTGGCAGATGGGAGAGC | 63.22 |
| PLK1 | gPLK1_7 | 1423 | CATGGACATCTTCTCCCTCTG | 90.07 |
| PLK1 | gPLK1_6 | 1422 | CCAAGTGCTTCGAGATCTCGG | 2.07 |
| PLK1 | gPLK1_1 | 1417 | CCAGGGTCGGCCGGTGCCCGT | 29.06 |
| PLK1 | gPLK1_9 | 1425 | CGAGGACAACGACTTCGTGTT | 6.84 |
| PLK1 | gPLK1_10 | 1426 | GAGGACAACGACTTCGTGTTC | 8.52 |
| PLK1 | gPLK1_2 | 1418 | GCCGGTGGAGCCGCCGCCGGA | 2.01 |
| PLK1 | gPLK1_5 | 1421 | GGCAAGGGCGGCTTTGCCAAG | 28.41 |
| PLK1 | gPLK1_4 | 1420 | GGGCAAGGGCGGCTTTGCCAA | 28.24 |
| PLK1 | gPLK1_8 | 1424 | TCGAGGACAACGACTTCGTGT | 0.16 |
| PLK1 | gPLK1_3 | 1419 | TGGGCAAGGGCGGCTTTGCCA | 2.26 |
| TUBB | gTUBB_1 | 1430 | AACCATGAGGGAAATCGTGCA | 2.61 |
| TUBB | gTUBB_2 | 1431 | ACCATGAGGGAAATCGTGCAC | 68.4 |
| TUBB | gTUBB_3 | 1432 | TTCTCTGTAGGTGGCAAATAT | 18.67 |
| U6 | gU6_5 | 1411 | ATATATCTTGTGGAAAGGACG | 0.39 |
| U6 | gU6_2 | 1408 | GATTTCTTGGCTTTATATATC | 0.71 |
| U6 | gU6_4 | 1410 | GCTTTATATATCTTGTGGAAA | 0.37 |
| U6 | gU6_1 | 1407 | GTCCTTTCCACAAGATATATA | 68.1 |
| U6 | gU6_6 | 1412 | TATATCTTGTGGAAAGGACGA | 0.39 |
| U6 | gU6_7 | 1413 | TGGAAAGGACGAAACACCGTG | 0.24 |
| U6 | gU6_3 | 1409 | TTGGCTTTATATATCTTGTGG | 2.83 |

To provide sufficient targeting to the target nucleotide sequence, the spacer sequence can be 16 or more nucleotides in length. In certain embodiments, the spacer sequence is at least 16, 17, 18, 19, 20, 21, 22, 23, 24, 25, 26, 27, 28, 29, 30, 35, 40, 45, 50, or 75 nucleotides in length. In certain embodiments, the spacer sequence is shorter than or equal to 75, 50, 45, 40, 35, 30, 25, or 20 nucleotides in length. Shorter spacer sequence may be desirable for reducing off-target events. Accordingly, in certain embodiments, the spacer sequence is shorter than or equal to 21, 20, 19, 18, or 17 nucleotides. In certain embodiments, the spacer sequence is 17-30 nucleotides in length, e.g., 17-21, 17-22, 17-23, 17-24, 17-25, 17-30, 20-21, 20-22, 20-23, 20-24, 20-25, or 20-30 nucleotides in length. In certain embodiments, the spacer sequence is about 20 nucleotides in length. In certain embodiments, the spacer sequence is about 21 nucleotides in length. In certain embodiments, the spacer sequence is 20 nucleotides in length.

In certain embodiments, the spacer sequence comprises a portion of a spacer sequence listed in Tables 7-9, wherein the portion is 16, 17, 18, 19, or 20 nucleotides in length. In certain embodiments, the spacer sequence comprises nucleotides 1-16, 1-17, 1-18, 1-19, or 1-20 of a spacer sequence listed in Tables 7-9. In specific embodiments, the spacer sequence consists of nucleotides 1-16, 1-17, 1-18, 1-19, or 1-20 of a spacer sequence listed in Tables 7-9.

In certain embodiments, the spacer sequence is 21 nucleotides in length. In certain embodiments, the spacer sequence consists of a spacer sequence shown in Tables 7-9.

In certain embodiments, the spacer sequence, where it is longer than 21 nucleotides in length, comprises a spacer sequence shown in Tables 7-9 and one or more nucleotides. In certain embodiments, the one or more nucleotides are 3′ to the spacer sequence shown in Tables 7-9.

In certain embodiments, the spacer sequence is at least 70%, at least 75%, at least 80%, at least 85%, at least 90%, at least 93%, at least 94%, at least 95%, at least 96%, at least 97%, at least 98%, or at least 99% complementary to the target nucleotide sequence. In certain embodiments, the spacer sequence is 100% complementary to the target nucleotide sequence in the seed region (about 5 base pairs proximal to the PAM). In certain embodiments, the spacer sequence is 100% complementary to the target nucleotide sequence. The spacer sequences listed in Tables 7-9 are designed to be 100% complementary to the wild-type sequence of the corresponding target gene. Accordingly, it is contemplated that a spacer sequence useful for targeting a gene listed in Tables 7-9 can be at least 70%, at least 75%, at least 80%, at least 85%, at least 90%, at least 93%, at least 94%, at least 95%, at least 96%, at least 97%, at least 98%, or at least 99% identical to a corresponding spacer sequence listed in Tables 7-9, or a portion thereof disclosed herein. In certain embodiments, the spacer sequence is 1, 2, 3, 4, 5, 6, 7, 8, 9, or 10 nucleotides different from a sequence listed in Tables 7-9. In certain embodiments, the spacer sequence is 100% identical to a sequence listed in Tables 7-9 in the seed region (about 5 base pairs proximal to the PAM). It has been reported that compared to DNA binding, DNA cleavage is less tolerant to mismatches between the spacer sequence and the target nucleotide sequence (see, Klein et al. (2018) Cell Reports, 22:1413). Accordingly, in certain embodiments, a guide nucleic acid to be used with a Cas nuclease comprises a spacer sequence 100% complementary to the target nucleotide sequence. In certain embodiments, a guide nucleic acid to be used with a Cas nuclease comprises a spacer sequence listed in Tables 7-9, or a portion thereof disclosed herein.

The present invention also provides guide nucleic acids targeting human DHODH, PLK1, MVD, TUBB, or U6 gene comprising the spacer sequences provided below in Table 10. DHODH, PLK1, MVD, and TUBB are known to be essential genes. It is contemplated that the guide nucleic acids targeting these genes, particularly the ones that edit the respective genomic locus at hight efficiency (e.g., at least 50%, at least 60%, at least 70%, at least 80%, or at least 90%), can be used as positive controls for assessing transfection efficiency and other experimental processes. The spacer sequences targeting U6 in Table 10 are designed to hybridize with the promoter region of human U6 gene and can be used to assess expression of an inserted gene from the endogenous U6 promoter.

### V. Pharmaceutical Compositions

Provided herein is a composition (e.g., pharmaceutical composition) comprising a guide nucleic acid, an engineered, non-naturally occurring system, or a eukaryotic cell, such as a guide nucleic acid, an engineered, non-naturally occurring system, or a eukaryotic cell, disclosed herein. In certain embodiments, the composition comprises an RNP comprising a guide nucleic acid, such as a guide nucleic acid disclosed herein, and a Cas protein (e.g., Cas nuclease). In certain embodiments, the composition comprises a single guide nucleic acid, such as a single guide nucleic acid disclosed herein. In certain embodiments, the composition comprises an RNP comprising the single guide nucleic acid, and a Cas protein (e.g., Cas nuclease). In certain embodiments, the composition comprises an RNP comprising the targeter nucleic acid, the modulator nucleic acid, and a Cas protein (e.g., Cas nuclease). In certain embodiments, the composition comprises a complex of a targeter nucleic acid and a modulator nucleic acid, such as a complex of a targeter nucleic acid and a modulator nucleic acid disclosed herein. In certain embodiments, the composition comprises an RNP comprising the targeter nucleic acid, the modulator nucleic acid, and a Cas protein (e.g., Cas nuclease).

In certain embodiments provided herein is a method of producing a composition, the method comprising incubating a single guide nucleic acid, such as a single guide nucleic acid disclosed herein, with a Cas protein, thereby producing a complex of the single guide nucleic acid and the Cas protein (e.g., an RNP). In certain embodiments, the method further comprises purifying the complex (e.g., the RNP).

In certain embodiments, provided is a method of producing a composition, the method comprising incubating a targeter nucleic acid and a modulator nucleic acid, such as a targeter nucleic acid and a modulator nucleic acid disclosed herein, under suitable conditions, thereby producing a composition (e.g., pharmaceutical composition) comprising a complex of the targeter nucleic acid and the modulator nucleic acid. In certain embodiments, the method further comprises incubating the targeter nucleic acid and the modulator nucleic acid with a Cas protein (e.g., the Cas nuclease that the targeter nucleic acid and the modulator nucleic acid are capable of activating or a related Cas protein), thereby producing a complex of the targeter nucleic acid, the modulator nucleic acid, and the Cas protein (e.g., an RNP). In certain embodiments, the method further comprises purifying the complex (e.g., the RNP).

For therapeutic use, a guide nucleic acid, an engineered, non-naturally occurring system, a CRISPR expression system, or a cell comprising such system or modified by such system disclosed herein is combined with a pharmaceutically acceptable carrier. The term “pharmaceutically acceptable” as used herein can refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit-to-risk ratio.

The term “pharmaceutically acceptable carrier” as used herein includes buffers, carriers, and excipients suitable for use in contact with the tissues of human beings and animals without excessive toxicity, irritation, allergic response, or other problem or complication, commensurate with a reasonable benefit/risk ratio. Pharmaceutically acceptable carriers include any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see, e.g., Martin, Remington's Pharmaceutical Sciences, 15th Ed., Mack Publ. Co., Easton, PA (1975). Pharmaceutically acceptable carriers include buffers, solvents, dispersion media, coatings, isotonic and absorption delaying agents, or the like, that are compatible with pharmaceutical administration. The use of such media and agents for pharmaceutically active substances is known in the art.

In certain embodiments, a pharmaceutical composition disclosed herein comprises a salt, e.g., NaCl, MgCl2, KCl, MgSO4, etc.: a buffering agent, e.g., a Tris buffer, N-(2-Hydroxyethyl)piperazine-N′-(2-ethanesulfonic acid) (HEPES), 2-(N-Morpholino)ethanesulfonic acid (MES), MES sodium salt, 3-(N-Morpholino) propanesulfonic acid (MOPS), N-tris[Hydroxymethyl] methyl-3-aminopropanesulfonic acid (TAPS), etc.; a solubilizing agent; a detergent, e.g., a non-ionic detergent such as Tween-20, etc.; a nuclease inhibitor; or the like. For example, in certain embodiments, a subject composition comprises a subject DNA-targeting RNA, e.g., gRNA, and a buffer for stabilizing nucleic acids.

In certain embodiments, a pharmaceutical composition may contain formulation materials for modifying, maintaining or preserving, for example, the pH, osmolarity, viscosity, clarity, color, isotonicity, odor, sterility, stability, rate of dissolution or release, adsorption or penetration of the composition. In such embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides: disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants (see, Remington's Pharmaceutical Sciences, 18th ed. (Mack Publishing Company, 1990).

In certain embodiments, a pharmaceutical composition may contain nanoparticles, e.g., polymeric nanoparticles, liposomes, or micelles (See Anselmo et al. (2016) Bioeng. Transl. Med. 1:10-29). In certain embodiment, the pharmaceutical composition comprises an inorganic nanoparticle. Exemplary inorganic nanoparticles include, e.g., magnetic nanoparticles (e.g., Fe3MnO2) or silica. The outer surface of the nanoparticle can be conjugated with a positively charged polymer (e.g., polyethylenimine, polylysine, polyserine) which allows for attachment (e.g., conjugation or entrapment) of payload. In certain embodiment, the pharmaceutical composition comprises an organic nanoparticle (e.g., entrapment of the payload inside the nanoparticle). Exemplary organic nanoparticles include, e.g., SNALP liposomes that contain cationic lipids together with neutral helper lipids which are coated with polyethylene glycol (PEG) and protamine and nucleic acid complex coated with lipid coating. In certain embodiment, the pharmaceutical composition comprises a liposome, for example, a liposome disclosed in International (PCT) Application Publication No. WO 2015/148863.

In certain embodiments, the pharmaceutical composition comprises a targeting moiety to increase target cell binding or update of nanoparticles and liposomes. Exemplary targeting moieties include cell specific antigens, monoclonal antibodies, single chain antibodies, aptamers, polymers, sugars, and cell penetrating peptides. In certain embodiments, the pharmaceutical composition comprises a fusogenic or endosome-destabilizing peptide or polymer.

In certain embodiments, a pharmaceutical composition may contain a sustained- or controlled-delivery formulation. Techniques for formulating sustained- or controlled-delivery means, such as liposome carriers, bio-erodible microparticles or porous beads and depot injections, are also known to those skilled in the art. Sustained-release preparations may include, e.g., porous polymeric microparticles or semipermeable polymer matrices in the form of shaped articles, e.g., films, or microcapsules. Sustained release matrices may include polyesters, hydrogels, polylactides, copolymers of L-glutamic acid and gamma ethyl-L-glutamate, poly (2-hydroxyethyl-inethacrylate), ethylene vinyl acetate, or poly-D(-)-3-hydroxybutyric acid. Sustained release compositions may also include liposomes that can be prepared by any of several methods known in the art.

A pharmaceutical composition of the invention can be administered by a variety of methods known in the art. The route and/or mode of administration vary depending upon the desired results. Administration can be intravenous, intramuscular, intraperitoneal, or subcutaneous, or administered proximal to the site of the target. The pharmaceutically acceptable carrier should be suitable for intravenous, intramuscular, subcutaneous, parenteral, spinal or epidermal administration (e.g., by injection or infusion). Depending on the route of administration, the active compound (e.g., the guide nucleic acid, engineered, non-naturally occurring system, or CRISPR expression system disclosed herein) may be coated in a material to protect the compound from the action of acids and other natural conditions that may inactivate the compound.

Formulation components suitable for parenteral administration include a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as EDTA; buffers such as acetates, citrates or phosphates; and agents for the adjustment of tonicity such as sodium chloride or dextrose.

For intravenous administration, suitable carriers include physiological saline, bacteriostatic water, Cremophor ELTM (BASF, Parsippany, NJ) or phosphate buffered saline (PBS). The carrier should be stable under the conditions of manufacture and storage, and should be preserved against microorganisms. The carrier can be a solvent or dispersion medium containing, for example, water, ethanol, polyol (for example, glycerol, propylene glycol, and liquid polyetheylene glycol), and suitable mixtures thereof.

Pharmaceutical formulations preferably are sterile. Sterilization can be accomplished by any suitable method, e.g., filtration through sterile filtration membranes. Where the composition is lyophilized, filter sterilization can be conducted prior to or following lyophilization and reconstitution. In certain embodiments, the pharmaceutical composition is lyophilized, and then reconstituted in buffered saline, at the time of administration.

Pharmaceutical compositions of the invention can be prepared in accordance with methods well known and routinely practiced in the art. See, e.g., Remington: The Science and Practice of Pharmacy, Mack Publishing Co., 20th ed., 2000; and Sustained and Controlled Release Drug Delivery Systems, J. R. Robinson, ed., Marcel Dekker, Inc., New York, 1978. Pharmaceutical compositions are preferably manufactured under GMP conditions. Typically, a therapeutically effective dose or efficacious dose of the guide nucleic acid, engineered, non-naturally occurring system, or CRISPR expression system disclosed herein is employed in the pharmaceutical compositions of the invention. The compositions disclosed herein are formulated into pharmaceutically acceptable dosage forms by conventional methods known to those of skill in the art. Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated: each unit contains a predetermined quantity of active compound calculated to produce the desired therapeutic effect in association with the required pharmaceutical carrier.

Actual dosage levels of the active ingredients in the pharmaceutical compositions of the invention can be varied so as to obtain an amount of the active ingredient which is effective to achieve the desired therapeutic response for a particular patient, composition, and mode of administration, without being toxic to the patient. The selected dosage level depends upon a variety of pharmacokinetic factors including the activity of the particular compositions disclosed herein employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors.

### VI. Therapeutic Uses

Guide nucleic acids, engineered, non-naturally occurring systems, and the CRISPR expression systems, e.g., as disclosed herein, are useful for targeting, editing, and/or modifying the genomic DNA in a cell or organism. These guide nucleic acids and systems, as well as a cell comprising one of the systems or a cell whose genome has been modified by one of the systems, can be used to treat a disease or disorder in which modification of genetic or epigenetic information is desirable. Accordingly, provided herein is a method of treating a disease or disorder, the method comprising administering to a subject in need thereof a guide nucleic acid, a non-naturally occurring system, a CRISPR expression system, or a cell disclosed herein.

The term “subject” includes human and non-human animals. Non-human animals include all vertebrates, e.g., mammals and non-mammals, such as non-human primates, sheep, dog, cow, chickens, amphibians, and reptiles. Except when noted, the terms “patient” or “subject” are used herein interchangeably.

The terms “treatment”, “treating”, “treat”, “treated”, or the like, as used herein, can refer to obtaining a desired pharmacologic and/or physiologic effect. The effect may be therapeutic in terms of a partial or complete cure for a disease and/or adverse effect attributable to the disease or delaying the disease progression. “Treatment”, as used herein, covers any treatment of a disease in a mammal, e.g., in a human, and includes: (a) inhibiting the disease, i.e., arresting its development; and (b) relieving the disease, i.e., causing regression of the disease. It is understood that a disease or disorder may be identified by genetic methods and treated prior to manifestation of any medical symptom.

For minimization of toxicity and off-target effect, it can be important to control the concentration of the CRISPR-Cas system delivered. Optimal concentrations can be determined by testing different concentrations in a cellular, tissue, or non-human eukaryote animal model and using deep sequencing to analyze the extent of modification at potential off-target genomic loci. The concentration that gives the highest level of on-target modification while minimizing the level of off-target modification is generally selected for ex vivo or in vivo delivery.

It is understood that the guide nucleic acid, the engineered, non-naturally occurring system, and the CRISPR expression system disclosed herein can be used to treat any suitable disease or disorder that can be improved by the system in a cell.

For therapeutic purposes, certain methods disclosed herein is particularly suitable for editing or modifying a proliferating cell, such as a stem cell (e.g., a hematopoietic stem cell), a progenitor cell (e.g., a hematopoietic progenitor cell or a lymphoid progenitor cell), or a memory cell (e.g., a memory T cell). Given that such cell is delivered to a subject and will proliferate in vivo, tolerance to off-target events is low. Prior to delivery, however, it is possible to assess the on-target and off-target events, thereby selecting one or more colonies that have the desired edit or modification and lack any undesired edit or modification. Therefore, lower editing or modifying efficiency can be tolerated for such cell. The engineered, non-naturally occurring system of the present invention has the advantage of increasing or decreasing the efficiency of nucleic acid cleavage by, for example, adjusting the hybridization of dual guide nucleic acids. As a result, it can be used to minimize off-target events when creating genetically engineered proliferating cells.

In certain embodiments, the guide nucleic acid, the engineered, non-naturally occurring system, and/or the CRISPR expression system disclosed herein can be used to engineer an immune cell. Immune cells include but are not limited to lymphocytes (e.g., B lymphocytes or B cells, T lymphocytes or T cells, and natural killer cells), myeloid cells (e.g., monocytes, macrophages, eosinophils, mast cells, basophils, and granulocytes), and the stem and progenitor cells that can differentiate into these cell types (e.g., hematopoietic stem cells, hematopoietic progenitor cells, and lymphoid progenitor cells). The cells can include autologous cells derived from a subject to be treated, or alternatively allogenic cells derived from a donor.

In certain embodiments, the immune cell is a T cell, which can be, for example, a cultured T cell, a primary T cell, a T cell from a cultured T cell line (e.g., Jurkat, SupTi), or a T cell obtained from a mammal, for example, from a subject to be treated. If obtained from a mammal, the T cell can be obtained from numerous sources, including but not limited to blood, bone marrow, lymph node, the thymus, or other tissues or fluids. T cells can also be enriched or purified. The T cell can be any type of T cell and can be of any developmental stage, including but not limited to, CD4⁺/CD8⁺ double positive T cells, CD4⁺ helper T cells (e.g., Th1 and Th2 cells), CD8⁺ T cells (e.g., cytotoxic T cells), tumor infiltrating lymphocytes (TILs), memory T cells (e.g., central memory T cells and effector memory T cells), regulatory T cells, naive T cells, or the like.

In certain embodiments, an immune cell, e.g., a T cell, is engineered to express an exogenous gene. For example, in certain embodiments, an engineered CRISPR system disclosed herein may catalyze DNA cleavage at the gene locus, allowing for site-specific integration of the exogenous gene at the gene locus by HDR.

In certain embodiments, an immune cell, e.g., a T cell, is engineered to express a chimeric antigen receptor (CAR), i.e., the T cell comprises an exogenous nucleotide sequence encoding a CAR. As used herein, the term “chimeric antigen receptor” or “CAR” includes any artificial receptor including an antigen-specific binding moiety and one or more signaling chains derived from an immune receptor. CARs can comprise a single chain fragment variable (scFv) of an antibody specific for an antigen coupled via hinge and transmembrane regions to cytoplasmic domains of T cell signaling molecules, e.g. a T cell costimulatory domain (e.g., from CD28, CD137, OX40, ICOS, or CD27) in tandem with a T cell triggering domain (e.g. from CD35). A T cell expressing a chimeric antigen receptor is referred to as a CAR T cell. Exemplary CAR T cells include CD19 targeted CTL019 cells (sec. Grupp et al. (2015) BLOOD. 126:4983), 19-28% cells (see, Park et al. (2015) J. CLIN. ONCOL., 33:7010), and KTE-C19 cells (sec. Locke et al. (2015) BLOOD. 126:3991). Additional exemplary CAR T cells are described in U.S. Pat. Nos. 7,446,190, 8,399,645, 8,906,682, 9,181,527, 9,272,002, 9,266,960, 10,253,086, 10,640,569, and 10,808,035, and International (PCT) Publication Nos. WO 2013/142034, WO 2015/120180, WO 2015/188141, WO 2016/120220, and WO 2017/040945. Exemplary approaches to express CARs using CRISPR systems are described in Hale et al. (2017) MOL THER METHODS CLIN DEV., 4:192. MacLeod et al. (2017) MOL THER. 25:949, and Eyquem et al. (2017) NATURE. 543:113.

In certain embodiments, an immune cell, e.g., a T cell, binds an antigen, e.g., a cancer antigen, through an endogenous T cell receptor (TCR). In certain embodiments, an immune cell. e.g., a T cell, is engineered to express an exogenous TCR, e.g., an exogenous naturally occurring TCR or an exogenous engineered TCR. T cell receptors comprise two chains referred to as the α- and β-chains, that combine on the surface of a T cell to form a heterodimeric receptor that can recognize MHC-restricted antigens. Each of α- and β-chain comprises a constant region and a variable region. Each variable region of the α- and β-chains defines three loops, referred to as complementary determining regions (CDRs) known as CDR₁, CDR₂, and CDR₃ that confer the T cell receptor with antigen binding activity and binding specificity.

In certain embodiments, a CAR or TCR binds a cancer antigen selected from B-cell maturation antigen (BCMA), mesothelin, prostate specific membrane antigen (PSMA), prostate stem cell antigen (PSCA), carbonic anhydrase IX (CAIX), carcinoembryonic antigen (CEA). CD5, CD7, CD10, CD19, CD20, CD22, CD30, CD33, CD34, CD38, CD41, CD44, CD49f, CD56, CD70, CD74, CD123, CD133, CD138, epithelial glycoprotein2 (EGP 2), epithelial glycoprotein-40 (EGP-40), epithelial cell adhesion molecule (EpCAM), receptor-type tyrosine-protein kinase (FLT3), folate-binding protein (FBP), fetal acetylcholine receptor (AChR), folate receptor-a and β (FRa and β), Ganglioside G2 (GD2), Ganglioside G3 (GD3), epidermal growth factor receptor 2 (HER-2/ERB2), epidermal growth factor receptor vIII (EGFRvIII), ERB3, ERB4, human telomerase reverse transcriptase (hTERT), Interleukin-13 receptor subunit alpha-2 (IL-13Ra2), K-light chain, kinase insert domain receptor (KDR), Lewis A (CA19.9), Lewis Y (LeY), LI cell adhesion molecule (LICAM), melanoma-associated antigen 1 (melanoma antigen family A1, MAGE-A1), Mucin 16 (MUC-16), Mucin 1 (MUC-1; e.g., a truncated MUC-1), KG2D ligands, cancer-testis antigen NY-ESO-1, oncofetal antigen (h5T4), tumor-associated glycoprotein 72 (TAG-72), vascular endothelial growth factor R2 (VEGF-R2), Wilms tumor protein (WT-1), type 1 tyrosine-protein kinase transmembrane receptor (ROR1), B7-H3 (CD276), B7-H6 (Nkp30), Chondroitin sulfate proteoglycan-4 (CSPG4), DNAX Accessory Molecule (DNAM-1), Ephrin type A Receptor 2 (EpHA2), Fibroblast Associated Protein (FAP), Gp100/HLA-A2, Glypican 3 (GPC3), HA-IH, HERK-V, IL-1 IRa, Latent Membrane Protein 1 (LMP1), Neural cell-adhesion molecule (N-CAM/CD56), and Trail Receptor (TRAIL-R).

Genetic loci suitable for insertion of a CAR- or exogenous TCR-encoding sequence include but are not limited to safe harbor loci (e.g., the AAVS1 locus) TCR subunit loci (e.g., the TCRα constant (TRAC) locus, the TCRβ constant 1 (TRBC1) locus, and the TCRβ constant 2 (TRBC2) locus). It is understood that insertion in the TRAC locus reduces tonic CAR signaling and enhances T cell potency (see, Eyquem et al. (2017) NATURE, 543:113). Furthermore, inactivation of the endogenous TRAC, TRBC1, or TRBC2 gene may reduce a graft-versus-host disease (GVHD) response, thereby allowing use of allogeneic T cells as starting materials for preparation of CAR-T cells. Accordingly, in certain embodiments, an immune cell, e.g., a T cell, is engineered to have reduced expression of an endogenous TCR or TCR subunit, e.g., TRAC, TRBC1, and/or TRBC2. The cell may be engineered to have partially reduced or no expression of the endogenous TCR or TCR subunit. For example, in certain embodiments, the immune cell, e.g., a T cell, is engineered to have less than 80% (e.g., less than 70%, less than 60%, less than 50%, less than 40%, less than 30%, less than 20%, less than 10%, or less than 5%) of the expression of the endogenous TCR or TCR subunit relative to a corresponding unmodified or parental cell. In certain embodiments, the immune cell, e.g., a T cell, is engineered to have no detectable expression of the endogenous TCR or TCR subunit. Exemplary approaches to reduce expression of TCRs using CRISPR systems are described in U.S. Pat. No. 9,181,527, Liu et al. (2017) CELL RES, 27:154, Ren et al. (2017) CLIN CANCER RES, 23:2255, Cooper et al. (2018) LEUKEMIA, 32:1970, and Ren et al. (2017) ONCOTARGET, 8:17002.

It is understood that certain immune cells, such as T cells, also express major histocompatibility complex (MHC) or human leukocyte antigen (HLA) genes, and inactivation of these endogenous gene may reduce an immune response, thereby allowing use of allogeneic T cells as starting materials for preparation of CAR-T cells. Accordingly, in certain embodiments, an immune cell, e.g., a T-cell, is engineered to have reduced expression of one or more endogenous class I or class II MHCs or HLAs (e.g., beta 2-microglobulin (B2M), class II major histocompatibility complex transactivator (CIITA)). The cell may be engineered to have partially reduced or no expression of an endogenous MHC or HLA. For example, in certain embodiments, the immune cell, e.g., a T-cell, is engineered to have less than less than 80% (e.g., less than 70%, less than 60%, less than 50%, less than 40%, less than 30%, less than 20%, less than 10%, or less than 5%) of the expression of endogenous MHC (e.g., B2M, CIITA) relative to a corresponding unmodified or parental cell. In certain embodiments, the immune cell, e.g., a T cell, is engineered to have no detectable expression of an endogenous MHC (e.g., B2M, CIITA). In certain cases, a cell may be engineered to have expression of, e.g., HLA-E and/or HLA-G, in order to avoid attack by natural killer (NK) cells. Exemplary approaches to reduce expression of MHCs using CRISPR systems are described in Liu et al. (2017) CELL RES, 27:154, Ren et al. (2017) CLIN CANCER RES, 23:2255, and Ren et al. (2017) ONCOTARGET, 8:17002.

Other genes that may be inactivated include but are not limited to CD3, CD52, and deoxycytidine kinase (DCK). For example, inactivation of DCK may render the immune cells (e.g., T cells) resistant to purine nucleotide analogue (PNA) compounds, which are often used to compromise the host immune system in order to reduce a GVHD response during an immune cell therapy. In certain embodiments, the immune cell, e.g., a T-cell, is engineered to have less than less than 80% (e.g., less than 70%, less than 60%, less than 50%, less than 40%, less than 30%, less than 20%, less than 10%, or less than 5%) of the expression of endogenous CD52 or DCK relative to a corresponding unmodified or parental cell.

It is understood that the activity of an immune cell (e.g., T cell) may be enhanced by inactivating or reducing the expression of an immune suppressor such as an immune checkpoint protein. Accordingly, in certain embodiments, an immune cell, e.g., a T cell, is engineered to have reduced expression of an immune checkpoint protein. Exemplary immune checkpoint proteins expressed by wild-type T cells include but are not limited to PDCD1 (PD-1), CTLA4, ADORA2A (A2AR), B7-H3, B7-H4, BTLA, KIR, LAG3, HAVCR2 (TIM3), TIGIT, VISTA, PTPN6 (SHP-1), and FAS. The cell may be modified to have partially reduced or no expression of the immune checkpoint protein. For example, in certain embodiments, the immune cell, e.g., a T cell, is engineered to have less than 80% (e.g., less than 70%, less than 60%, less than 50%, less than 40%, less than 30%, less than 20%, less than 10%, or less than 5%) of the expression of the immune checkpoint protein relative to a corresponding unmodified or parental cell. In certain embodiments, the immune cell, e.g., a T cell, is engineered to have no detectable expression of the immune checkpoint protein. Exemplary approaches to reduce expression of immune checkpoint proteins using CRISPR systems are described in International (PCT) Publication No. WO 2017/017184, Cooper et al. (2018) LEUKEMIA, 32:1970, Su et al. (2016) ONCOIMMUNOLOGY, 6:1249558, and Zhang et al. (2017) FRONT MED, 11:554.

The immune cell can be engineered to have reduced expression of an endogenous gene, e.g., an endogenous genes described above, by gene editing or modification. For example, in certain embodiments, an engineered CRISPR system disclosed herein may result in DNA cleavage at a gene locus, thereby inactivating the targeted gene. In other embodiments, an engineered CRISPR system disclosed herein may be fused to an effector domain (e.g., a transcriptional repressor or histone methylase) to reduce the expression of the target gene.

The immune cell can also be engineered to express an exogenous protein (besides an antigen-binding protein described above) at the locus of a human ADORA2A, B2M, CD52, CIITA, CTLA4, DCK, FAS, HAVCR2, LAG3, PDCD1, PTPN6, TIG1T, TRAC, TRBC1, TRBC2, CARD11, CD247, IL7R, LCK, or PLCG1 gene.

In certain embodiments, an immune cell, e.g., a T cell, is modified to express a dominant-negative form of an immune checkpoint protein. In certain embodiments, the dominant-negative form of the checkpoint inhibitor can act as a decoy receptor to bind or otherwise sequester the natural ligand that would otherwise bind and activate the wild-type immune checkpoint protein. Examples of engineered immune cells, for example, T cells containing dominant-negative forms of an immune suppressor are described, for example, in International (PCT) Publication No. WO 2017/040945.

In certain embodiments, an immune cell, e.g., a T cell, is modified to express a gene (e.g., a transcription factor, a cytokine, or an enzyme) that regulates the survival, proliferation, activity, or differentiation (e.g., into a memory cell) of the immune cell. In certain embodiments, the immune cell is modified to express TET2, FOXO1, IL-12, IL-15, IL-18, IL-21, IL-7, GLUT1, GLUT3, HK1, HK2, GAPDH, LDHA, PDK1, PKM2, PFKFB3, PGK1, ENO1, GYSI, and/or ALDOA. In certain embodiments, the modification is an insertion of a nucleotide sequence encoding the protein operably linked to a regulatory element. In certain embodiments, the modification is a substitution of a single nucleotide polymorphism (SNP) site in the endogenous gene. In certain embodiments, an immune cell, e.g., a T cell, is modified to express a variant of a gene, for example, a variant that has greater activity than the respective wild-type gene. In certain embodiments, the immune cell is modified to express a variant of CARDI1, CD247, IL7R, LCK, or PLCG1. For example, certain gain-of-function variants of IL7R were disclosed in Zenatti et al., (2011) NAT. GENET. 43 (10): 932-39. The variant can be expressed from the native locus of the respective wild-type gene by delivering an engineered system described herein for targeting the native locus in combination with a donor template that carries the variant or a portion thereof.

In certain embodiments, an immune cell, e.g., a T cell, is modified to express a protein (e.g., a cytokine or an enzyme) that regulates the microenvironment that the immune cell is designed to migrate to (e.g., a tumor microenvironment). In certain embodiments, the immune cell is modified to express CA9, CA12, a V-ATPase subunit, NHE1, and/or MCT-1.

### A. Gene Therapies

It is understood that the engineered, non-naturally occurring system and CRISPR expression system, e.g., as disclosed herein, can be used to treat a genetic disease or disorder, i.e., a disease or disorder associated with or otherwise mediated by an undesirable mutation in the genome of a subject.

Exemplary genetic diseases or disorders include age-related macular degeneration, adrenoleukodystrophy (ALD), Alagille syndrome, alpha-1-antitrypsin deficiency, argininemia, argininosuccinic aciduria, ataxia (e.g., Friedreich ataxia, spinocerebellar ataxias, ataxia telangiectasia, essential tremor, spastic paraplegia), autism, biliary atresia, biotinidase deficiency, carbamoyl phosphate synthetase I deficiency, carbohydrate deficient glycoprotein syndrome (CDGS), a central nervous system (CNS)-related disorder (e.g., Alzheimer's disease, amyotrophic lateral sclerosis (ALS), canavan disease (CD), ischemia, multiple sclerosis (MS), neuropathic pain, Parkinson's disease), Bloom's syndrome, cancer, Charcot-Marie-Tooth disease (e.g., peroneal muscular atrophy, hereditary motor sensory neuropathy), congenital hepatic porphyria, citrullinemia, Crigler-Najjar syndrome, cystic fibrosis (CF), Dentatorubro-Pallidoluysian Atrophy (DRPLA), diabetes insipidus, Fabry, familial hypercholesterolemia (LDL receptor defect), Fanconi's anemia, fragile X syndrome, a fatty acid oxidation disorder, galactosemia, glucose-6-phosphate dehydrogenase (G6PD), glycogen storage diseases (e.g., type I (glucose-6-phosphatase deficiency, Von Gierke II (alpha glucosidase deficiency, Pompe), III (debrancher enzyme deficiency, Cori), IV (brancher enzyme deficiency, Anderson), V (muscle glycogen phosphorylase deficiency, McArdle), VII (muscle phosphofructokinase deficiency, Tauri), VI (liver phosphorylase deficiency, Hers), IX (liver glycogen phosphorylase kinase deficiency)), hemophilia A (associated with defective factor VIII), hemophilia B (associated with defective factor IX), Huntington's disease, glutaric aciduria, hypophosphatemia, Krabbe, lactic acidosis, Lafora disease, Leber's Congenital Amaurosis, Lesch Nyhan syndrome, a lysosomal storage disease, metachromatic leukodystrophy disease (MLD), mucopolysaccharidosis (MPS) (e.g., Hunter syndrome, Hurler syndrome, Maroteaux-Lamy syndrome, Sanfilippo syndrome, Scheie syndrome, Morquio syndrome, other, MPSI, MPSII, MPSIII, MSIV, MPS 7), a muscular/skeletal disorder (e.g., muscular dystrophy, Duchenne muscular dystrophy), myotonic Dystrophy (DM), neoplasia, N-acetylglutamate synthase deficiency, ornithine transcarbamylase deficiency, phenylketonuria, primary open angle glaucoma, retinitis pigmentosa, schizophrenia, Severe Combined Immune Deficiency (SCID), Spinobulbar Muscular Atrophy (SBMA), sickle cell anemia, Usher syndrome, Tay-Sachs disease, thalassemia (e.g., β-Thalassemia), trinucleotide repeat disorders, tyrosinemia, Wilson's disease, Wiskott-Aldrich syndrome, X-linked chronic granulomatous disease (CGD), X-linked severe combined immune deficiency, and xeroderma pigmentosum.

Additional exemplary genetic diseases or disorders and associated information are available on the world wide web at kumc.edu/gec/support, genome.gov/10001200, and ncbi.nlm.nih.gov/books/NBK22183/. Additional exemplary genetic diseases or disorders, associated genetic mutations, and gene therapy approaches to treat genetic diseases or disorders are described in International (PCT) Publication Nos. WO 2013/126794, WO 2013/163628, WO 2015/048577, WO 2015/070083, WO 2015/089354, WO 2015/134812, WO 2015/138510, WO 2015/148670, WO 2015/148860, WO 2015/148863, WO 2015/153780, WO 2015/153789, and WO 2015/153791, U.S. Pat. Nos. 8,383,604, 8,859,597, 8,956,828, 9,255,130, and 9,273,296, and U.S. Patent Application Publication Nos. 2009/0222937, 2009/0271881, 2010/0229252, 2010/0311124, 2011/0016540, 2011/0023139, 2011/0023144, 2011/0023145, 2011/0023146, 2011/0023153, 2011/0091441, 2012/0159653, and 2013/0145487.

### B. Immune Cell Engineering

It is understood that the engineered, non-naturally occurring systems comprising ssODNs disclosed herein can be used to engineer an immune cell. Immune cells include but are not limited to lymphocytes (e.g., B lymphocytes or B cells, T lymphocytes or T cells, and natural killer cells), myeloid cells (e.g., monocytes, macrophages, cosinophils, mast cells, basophils, and granulocytes), and the stem and progenitor cells that can differentiate into these cell types (e.g., hematopoietic stem cells, hematopoietic progenitor cells, and lymphoid progenitor cells). The cells can include autologous cells derived from a subject to be treated, or alternatively allogenic cells derived from a donor.

It is understood that CRISPR systems comprising ssODNs disclosed herein can be used to treat any disease or disorder that can be improved by editing or modifying a target sequence: exemplary genes containing target sequences to be modified for therapeutic purposes include ADORA2A, ALPNR, B2M, BBS1, CALR, CARD11, CD3E, CD3G, CD38, CD40LG, CD52, CD58, CD247, CIITA, COL17A1, CSF1R, CSF2, CTLA4, DCK, DEFB134, DHODH, ERAP1, ERAP2, FAS, mir-101-2, HAVCR2 (also called TIM3), IFNGR1, IFNGR2, IL7R, JAK1, JAK2, LAG3, LCK, LCK1, MLANA, MVD, PDCD1 (also called PD-1), PLCG1, PLK1, PSMB5, PSMB8, PSMB9, PTCD2, PTPN1, PTPN6, PTPN11, RFX5, RFXAP, RPL23, RXANK, SOX10, SRP54, STAT1, Tap1, TAP2, TAPBP, TGFBR2, TIGIT, TIM3, TRAC, TRBC1, TRBC1+2, TRBC2, TUBB, TWF1, and/or U6 gene in a cell.

In certain embodiments, the immune cell is a T cell, which can be, for example, a cultured T cell, a primary T cell, a T cell from a cultured T cell line (e.g., Jurkat, SupTi), or a T cell obtained from a mammal, for example, from a subject to be treated. If obtained from a mammal, the T cell can be obtained from numerous sources, including but not limited to blood, bone marrow, lymph node, the thymus, or other tissues or fluids. T cells can also be enriched or purified. The T cell can be any type of T cell and can be of any developmental stage, including but not limited to, CD4+/CD8+double positive T cells, CD4+ helper T cells (e.g., Th1 and Th2 cells), CD8+ T cells (e.g., cytotoxic T cells), tumor infiltrating lymphocytes (TILs), memory T cells (e.g., central memory T cells and effector memory T cells), regulatory T cells, naive T cells, and the like.

In certain embodiments, an immune cell, e.g., a T cell, is engineered to express an exogenous gene. For example, in certain embodiments, an engineered CRISPR system disclosed herein may be used to engineer an immune cell to express an exogenous gene. For example, in certain embodiments, the guide nucleic acid, the engineered, non-naturally occurring system, and the CRISPR expression system disclosed herein may be used to engineer an immune cell to express an exogenous gene at the locus of a human ADORA2A, ALPNR, B2M, BBS1, CALR, CARD11, CD3E, CD3G, CD38, CD40LG, CD52, CD58, CD247, CIITA, COL17A1, CSF1R, CSF2, CTLA4, DCK, DEFB134, DHODH, ERAP1, ERAP2, FAS, mir-101-2, HAVCR2 (also called TIM3), IFNGR1, IFNGR2, IL7R, JAKI, JAK2, LAG3, LCK, LCK1, MLANA, MVD, PDCD1 (also called PD-1), PLCG1, PLK1, PSMB5, PSMB8, PSMB9, PTCD2, PTPN1, PTPN6, PTPN11, RFX5, RFXAP, RPL23, RXANK, SOX10, SRP54, STAT1, Tap1, TAP2, TAPBP, TGFBR2, TIGIT, TIM3, TRAC, TRBC1, TRBC1+2, TRBC2, TUBB, TWF1, and/or U6 gene. For example, in certain embodiments, an engineered CRISPR system comprising ssODNs disclosed herein may catalyze DNA cleavage at a gene locus, allowing for site-specific integration of the exogenous gene at the gene locus by HDR, while decreasing off-target effects by incorporating wild-type gene back into off-target cleaved sites by HDR.

In certain embodiments, an immune cell, e.g., a T cell, is engineered to express a chimeric antigen receptor (CAR), i.e., the T cell comprises an exogenous nucleotide sequence encoding a CAR. As used herein, the term “chimeric antigen receptor” or “CAR” includes any artificial receptor including an antigen-specific binding moiety and one or more signaling chains derived from an immune receptor. CARs can comprise a single chain fragment variable (scFv) of an antibody specific for an antigen coupled via hinge and transmembrane regions to cytoplasmic domains of T cell signaling molecules, e.g. a T cell costimulatory domain (e.g., from CD28, CD137, OX40, ICOS, or CD27) in tandem with a T cell triggering domain (e.g. from CD3(E∂). A T cell expressing a chimeric antigen receptor is referred to as a CAR T cell. Exemplary CAR T cells include CD19 targeted CTL019 cells (see. Grupp et al. (2015) BLOOD. 126:4983). 19-282 cells (see, Park et al. (2015) J. CLIN. ONCOL., 33:7010), and KTE-C19 cells (see. Locke et al. (2015) BLOOD, 126:3991). Additional exemplary CAR T cells are described in U.S. Pat. Nos. 8,399,645, 8,906,682, 7,446,190, 9,181,527, 9,272,002, and 9,266,960, U.S. Patent Publication Nos. 2016/0362472, 2016/0200824, and 2016/0311917, and International (PCT) Publication Nos. WO2013/142034, WO2015/120180, WO2015/188141, WO2016/120220, and WO2017/040945. Exemplary approaches to express CARs using CRISPR systems are described in Hale et al. (2017) MOL THER METHODS CLIN DEV., 4:192. Macleod et al. (2017) MOL THER, 25:949, and Eyquem et al. (2017) NATURE. 543:113.

In certain embodiments, an immune cell, e.g., a T cell, binds an antigen, e.g., a cancer antigen, through an endogenous T cell receptor (TCR). In certain embodiments, an immune cell, e.g., a T cell, is engineered to express an exogenous TCR, e.g., an exogenous naturally occurring TCR or an exogenous engineered TCR. T cell receptors comprise two chains referred to as the α- and β-chains, that combine on the surface of a T cell to form a heterodimeric receptor that can recognize MHC-restricted antigens. Each of α- and β-chain comprises a constant region and a variable region. Each variable region of the α- and β-chains defines three loops, referred to as complementary determining regions (CDRs) known as CDR1, CDR2, and CDR3 that confer the T cell receptor with antigen binding activity and binding specificity.

In certain embodiments, a CAR or TCR binds a cancer antigen selected from B-cell maturation antigen (BCMA), mesothelin, prostate specific membrane antigen (PSMA), prostate stem cell antigen (PCSA), carbonic anhydrase IX (CAIX), carcinoembryonic antigen (CEA). CD5, CD7, CD10, CD19, CD20, CD22, CD30, CD33, CD34, CD38, CD41, CD44, CD49f, CD56, CD70, CD74, CD123, CD133, CD138, epithelial glycoprotein2 (EGP 2), epithelial glycoprotein-40 (EGP-40), epithelial cell adhesion molecule (EpCAM), receptor-type tyrosine-protein kinase (FLT3), folate-binding protein (FBP), fetal acetylcholine receptor (AChR), folate receptor-α and β (FRα and β), Ganglioside G2 (GD2), Ganglioside G3 (GD3), epidermal growth factor receptor 2 (HER-2/ERB2), epidermal growth factor receptor vIII (EGFRvIII), ERB3. ERB4, human telom erase reverse transcriptase (hTERT). Interleukin-13 receptor subunit alpha-2 (IL-13Ra2), K-light chain, kinase insert domain receptor (KDR), Lewis A (CA19.9), Lewis Y (LeY), LI cell adhesion molecule (LICAM), melanoma-associated antigen 1 (melanoma antigen family Al, MAGE-A1), Mucin 16 (MUC-16), Mucin 1 (MUC-1; e.g., a truncated MUC-1), KG2D ligands, cancer-testis antigen NY-ESO-1, oncofetal antigen (h5T4), tumor-associated glycoprotein 72 (TAG-72), vascular endothelial growth factor R2 (VEGF-R2), Wilms tumor protein (WT-1), type 1 tyrosine-protein kinase transmembrane receptor (ROR1), B7-H3 (CD276), B7-H6 (Nkp30), Chondroitin sulfate proteoglycan-4 (CSPG4), DNAX Accessory Molecule (DNAM-1), Ephrin type A Receptor 2 (EpHA2), Fibroblast Associated Protein (FAP), Gp100/HLA-A2, Glypican 3 (GPC3), HA-IH, HERK-V, IL-1 IRa, Latent Membrane Protein 1 (LMP1), Neural cell-adhesion molecule (N-CAM/CD56), and Trail Receptor (TRAIL-R).

Genetic loci suitable for insertion of a CAR- or exogenous TCR-encoding sequence include but are not limited to safe harbor loci (e.g., the AAVS1 locus), TCR subunit loci (e.g., the TCRα constant (TRAC) locus), and other loci associated with certain advantages (e.g., the CCR5 locus, the inactivation of which may prevent or reduce HIV infection). It is understood that insertion in the TRAC locus reduces tonic CAR signaling and enhances T cell potency (see, Eyquem et al. (2017) NATURE, 543:113). Furthermore, inactivation of the endogenous TRAC gene may reduce a graft-versus-host disease (GVHD) response, thereby allowing use of allogeneic T cells as starting materials for preparation of CAR-T cells. Accordingly, in certain embodiments, an immune cell, e.g., a T cell, is engineered to have reduced expression of an endogenous TCR or TCR subunit, e.g., TCRα subunit constant (TRAC). The cell may be engineered to have partially reduced or no expression of the endogenous TCR or TCR subunit. For example, in certain embodiments, the immune cell, e.g., a T cell, is engineered to have less than 80% (e.g., less than 70%, less than 60%, less than 50%, less than 40%, less than 30%, less than 20%, less than 10%, or less than 5%) of the expression of the endogenous TCR or TCR subunit relative to a corresponding unmodified or parental cell. In certain embodiments, the immune cell, e.g., a T cell, is engineered to have no detectable expression of the endogenous TCR or TCR subunit. Exemplary approaches to reduce expression of TCRs using CRISPR systems are described in U.S. Pat. No. 9,181,527, Liu et al. (2017) CELL RES, 27:154, Ren et al. (2017) CLIN CANCER RES, 23:2255, Cooper et al. (2018) LEUKEMIA, 32:1970, and Ren et al. (2017) ONCOTARGET, 8:17002.

It is understood that certain immune cells, such as T cells, also express major histocompatibility complex (MHC) or human leukocyte antigen (HLA) genes, and inactivation of these endogenous gene may reduce a GVHD response, thereby allowing use of allogeneic T cells as starting materials for preparation of CAR-T cells. Accordingly, in certain embodiments, an immune cell, e.g., a T-cell, is engineered to have reduced expression of one or more endogenous class I or class II MHCs or HLAs (e.g., beta 2-microglobulin (B2M), class II major histocompatibility complex transactivator (CIITA), HLA-E, and/or HLA-G). The cell may be engineered to have partially reduced or no expression of an endogenous MHC or HLA. For example, in certain embodiments, the immune cell, e.g., a T-cell, is engineered to have less than less than 80% (e.g., less than 70%, less than 60%, less than 50%, less than 40%, less than 30%, less than 20%, less than 10%, or less than 5%) of the expression of endogenous MHC (e.g., B2M, CIITA, HLA-E, or HLA-G) relative to a corresponding unmodified or parental cell. In certain embodiments, the immune cell, e.g., a T cell, is engineered to have no detectable expression of an endogenous MHC (e.g., B2M, CIITA, HLA-E, or HLA-G). Exemplary approaches to reduce expression of MHCs using CRISPR systems are described in Liu et al. (2017) CELL RES, 27:154, Ren et al. (2017) CLIN CANCER RES, 23:2255, and Ren et al. (2017) ONCOTARGET, 8:17002.

Other genes that may be inactivated to reduce a GVHD response include but are not limited to CD3, CD52, and deoxycytidine kinase (DCK). For example, inactivation of DCK may render the immune cells (e.g., T cells) resistant to purine nucleotide analogue (PNA) compounds, which are often used to compromise the host immune system in order to reduce a GVHD response during an immune cell therapy. In certain embodiments, the immune cell, e.g., a T-cell, is engineered to have less than less than 80% (e.g., less than 70%, less than 60%, less than 50%, less than 40%, less than 30%, less than 20%, less than 10%, or less than 5%) of the expression of endogenous CD52 or DCK relative to a corresponding unmodified or parental cell.

In certain embodiments, an immune cell, e.g., a T cell, is engineered to have reduced expression of an endogenous gene. For example, in certain embodiments, an engineered CRISPR system disclosed herein may be used to engineer an immune cell to have reduced expression of an endogenous gene. For example, in certain embodiments, an engineered CRISPR system disclosed herein may result in DNA cleavage at a gene locus, thereby inactivating the targeted gene. In other embodiments, an engineered CRISPR system disclosed herein may be fused to an effector domain (e.g., a transcriptional repressor or histone methylase) to reduce the expression of the target gene.

It is understood that the activity of an immune cell (e.g., T cell) may be enhanced by inactivating or reducing the expression of an immune suppressor such as an immune checkpoint protein. Accordingly, in certain embodiments, an immune cell, e.g., a T cell, is engineered to have reduced expression of an immune checkpoint protein. Exemplary immune checkpoint proteins expressed by wild-type T cells include but are not limited to PDCD1 (PD-1), CTLA4, ADORA2A (A2AR), B7-H3, B7-H4, BTLA, KIR, LAG3, HAVCR2 (TIM3), TIGIT, VISTA, PTPN6 (SHP-1), and FAS. The cell may be modified to have partially reduced or no expression of the immune checkpoint protein. For example, in certain embodiments, the immune cell, e.g., a T cell, is engineered to have less than 80% (e.g., less than 70%, less than 60%, less than 50%, less than 40%, less than 30%, less than 20%, less than 10%, or less than 5%) of the expression of the immune checkpoint protein relative to a corresponding unmodified or parental cell. In certain embodiments, the immune cell, e.g., a T cell, is engineered to have no detectable expression of the immune checkpoint protein. Exemplary approaches to reduce expression of immune checkpoint proteins using CRISPR systems are described in International (PCT) Publication No. WO2017/017184, Cooper et al. (2018) LEUKEMIA, 32:1970, Su et al. (2016) ONCOIMMUNOLOGY, 6: e1249558, and Zhang et al. (2017) FRONT MED, 11:554.

The immune cell can also be engineered to express an exogenous protein (besides an antigen-binding protein described above) at the locus of a human ADORA2A, ALPNR, B2M, BBS1, CALR, CARD11, CD3E, CD3G, CD38, CD40LG, CD52, CD58, CD247, CIITA, COL17A1, CSF1R, CSF2, CTLA4, DCK, DEFB134, DHODH, ERAP1, ERAP2, FAS, mir-101-2, HAVCR2 (also called TIM3), IFNGR1, IFNGR2, IL7R, JAK1, JAK2, LAG3, LCK, LCK1, MLANA, MVD, PDCD1 (also called PD-1), PLCG1, PLK1, PSMB5, PSMB8, PSMB9, PTCD2, PTPN1, PTPN6, PTPN11, RFX5, RFXAP, RPL23, RXANK, SOX10, SRP54, STAT1, Tap1, TAP2, TAPBP, TGFBR2, TIG1T, TIM3, TRAC, TRBC1, TRBC1+2, TRBC2, TUBB, TWFI, and/or U6 gene.

In certain embodiments, an immune cell, e.g., a T cell, is modified to express a dominant-negative form of an immune checkpoint protein. In certain embodiments, the dominant-negative form of the checkpoint inhibitor can act as a decoy receptor to bind or otherwise sequester the natural ligand that would otherwise bind and activate the wild-type immune checkpoint protein. Examples of engineered immune cells, for example, T cells containing dominant-negative forms of an immune suppressor are described, for example, in International (PCT) Publication No. WO2017/040945.

In certain embodiments, an immune cell, e.g., a T cell, is modified to express a gene (e.g., a transcription factor, a cytokine, or an enzyme) that regulates the survival, proliferation, activity, or differentiation (e.g., into a memory cell) of the immune cell. In certain embodiments, the immune cell is modified to express TET2, FOXO1, IL-12, IL-15, IL-18, IL-21, IL-7, GLUT1, GLUT3, HK1, HK2, GAPDH, LDHA, PDK1, PKM2, PFKFB3, PGK1, ENO1, GYS1, and/or ALDOA. In certain embodiments, the modification is an insertion of a nucleotide sequence encoding the protein operably linked to a regulatory element. In certain embodiments, the modification is a substitution of a single nucleotide polymorphism (SNP) site in the endogenous gene. In certain embodiments, an immune cell, e.g., a T cell, is modified to express a variant of a gene, for example, a variant that has greater activity than the respective wild-type gene. In certain embodiments, the immune cell is modified to express a variant of CARD11, CD247, IL7R, LCK, or PLCG1. For example, certain gain-of-function variants of IL7R were disclosed in Zenatti et al., (2011) NAT. GENET. 43 (10): 932-39. The variant can be expressed from the native locus of the respective wild-type gene by delivering an engineered system described herein for targeting the native locus in combination with a donor template that carries the variant or a portion thereof.

In certain embodiments, an immune cell, e.g., a T cell, is modified to express a protein (e.g., a cytokine or an enzyme) that regulates the microenvironment that the immune cell is designed to migrate to (e.g., a tumor microenvironment). In certain embodiments, the immune cell is modified to express CA9, CA12, a V-ATPase subunit, NHE1, and/or MCT-1.

In certain embodiments, provided is a method for treatment of a disease, e.g., a cancer, by administering to a subject suffering from the disease an effective amount of T cells modified to express a CAR specific to the disease using the modified guide nucleic acids and CRISPR-Cas systems described herein, e.g., in sections IA, IA1, IB, IC, and IVB. In certain embodiments, the T cells are autologous cells removed from the subject, treated to modify genomic DNA to express CAR, expanded, and administered to the subject: in certain embodiments, the T cells are allogeneic T cells that have been treated to modify genomic DNA to express CAR. In certain embodiments, the disease is a blood cancer, such as leukemia or lymphoma; in certain embodiments the disease is a solid tumor cancer.

### VII. Kits

It is understood that the guide nucleic acid, the engineered, non-naturally occurring system, the CRISPR expression system, and/or a library disclosed herein can be packaged in a kit suitable for use by a medical provider. Accordingly, in another aspect, the invention provides kits containing any one or more of the elements disclosed in the above systems, libraries, methods, and compositions. In certain embodiments, the kit comprises an engineered, non-naturally occurring system as disclosed herein and instructions for using the kit. The instructions may be specific to the applications and methods described herein. In certain embodiments, one or more of the elements of the system are provided in a solution. In certain embodiments, one or more of the elements of the system are provided in lyophilized form, and the kit further comprises a diluent. Elements may be provided individually or in combinations, and may be provided in any suitable container, such as a vial, a bottle, a tube, or immobilized on the surface of a solid base (e.g., chip or microarray). In certain embodiments, the kit comprises one or more of the nucleic acids and/or proteins described herein. In certain embodiments, the kit provides all elements of the systems of the invention.

In certain embodiments of a kit comprising the engineered, non-naturally occurring dual guide system, the targeter nucleic acid and the modulator nucleic acid are provided in separate containers. In other embodiments, the targeter nucleic acid and the modulator nucleic acid are pre-complexed, and the complex is provided in a single container.

In certain embodiments, the kit comprises a Cas protein or a nucleic acid comprising a regulatory element operably linked to a nucleic acid encoding a Cas protein provided in a separate container. In other embodiments, the kit comprises a Cas protein pre-complexed with the single guide nucleic acid or a combination of the targeter nucleic acid and the modulator nucleic acid, and the complex is provided in a single container.

In certain embodiments, the kit further comprises one or more donor templates provided in one or more separate containers. In certain embodiments, the kit comprises a plurality of donor templates as disclosed herein (e.g., in separate tubes or immobilized on the surface of a solid base such as a chip or a microarray), one or more guide nucleic acids disclosed herein, and optionally a Cas protein or a regulatory element operably linked to a nucleic acid encoding a Cas protein as disclosed herein. Such kits are useful for identifying a donor template that introduces optimal genetic modification in a multiplex assay. The CRISPR expression systems as disclosed herein are also suitable for use in a kit.

In certain embodiments, a kit further comprises one or more reagents and/or buffers for use in a process utilizing one or more of the elements described herein. Reagents may be provided in any suitable container and may be provided in a form that is usable in a particular assay, or in a form that requires addition of one or more other components before use (e.g., in concentrate or lyophilized form). A buffer may be a reaction or storage buffer, including but not limited to a sodium carbonate buffer, a sodium bicarbonate buffer, a borate buffer, a Tris buffer, a MOPS buffer, a HEPES buffer, and combinations thereof. In some embodiments, the buffer is alkaline. In certain embodiments, the buffer has a pH from about 7 to about 10. In certain embodiments, the kit further comprises a pharmaceutically acceptable carrier. In certain embodiments, the kit further comprises one or more devices or other materials for administration to a subject.

### VIII. Embodiments

In embodiment 1 provided herein is a composition comprising a plurality of ssODNs wherein each of the ssODNs comprises a sequence that is complementary to and specific for a sequence flanking a strand break at an off-target site for a nucleic acid-guided nuclease complex comprising a nucleic acid-guided nuclease and a guide nucleic acid (gNA) wherein the ssODNs each comprise different sequences for different off-target sites. In embodiment 2 provided herein is the composition of claim 1 further comprising the nucleic acid-guided nuclease and gNA. In embodiment 3 provided herein is the composition of embodiment 1 or embodiment 2 wherein each ssODN further comprises a sequence coding for a wild-type gene at the off-target site. In embodiment 4 provided herein is the composition of any previous embodiment wherein at least 10, 20, 30, 40, 50, 60, 70, 80, 90, 95, 99, or 100% of the ssODNs comprise at least one mutation compared to the wild-type sequence. In embodiment 5 provided herein is the composition of embodiment 4 wherein the mutation comprises a mutation to a PAM. In embodiment 6 provided herein is the composition of embodiment 5 wherein the mutation to the PAM decreases or eliminates recognition of the off-target site by the nucleic acid-guided nuclease complex. In embodiment 7 provided herein is the composition of any previous embodiment further comprising a HDR enhancer. In embodiment 8 provided herein is the composition of embodiment 7 wherein the HDR enhancer comprises M3814. In embodiment 9 provided herein is the composition of embodiment 8 wherein the M3814 is present at a concentration of at least 0.1, 0.25, 0.5, 0.75, 1, 1.25, 1.5, 1.75, 2, 2.5, 3, or 4 and/or not more than 0.25, 0.5, 0.75, 1, 1.25, 1.5, 1.75, 2, 2.5, 3, 4, or 5 μM, for example 0.1-5 μM. In embodiment 10 provided herein is the composition of any previous embodiment further comprising an anionic polymer. In embodiment 11 provided herein is the composition of embodiment 10 wherein the anionic polymer comprises a non-specific ssODN or a peptide. In embodiment 12 provided herein is the composition of embodiment 11 wherein the peptide comprises poly-L-glutamic acid (PGA). In embodiment 13 provided herein is the composition of embodiment 11 comprising at least 50, 60, 70, 80, 90, 100, 150, 200, 250, 300, 350, 400, 450, 500, 550, 600, 700, 800, or 900 and/or not more than 60, 70, 80, 90, 100, 150, 200, 250, 300, 350, 400, 450, 500, 550, 600, 700, 800, 900 or 1000 pmol non-specific ssODN, for example 50-1000 pmol non-specific ssODN. In embodiment 14 provided herein is the composition of embodiment 12 wherein the PGA is present at a concentration of at least 0.01, 0.05, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.5, 2, 2.5, 3, 3.5, 4, or 4.5 and/or not more than 0.05, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.5, 2, 2.5, 3, 3.5, 4, 4.5, or 5 μg μL⁻¹ per pmol nucleic acid-guided nuclease complex, for example 0.01-5 μg μL⁻¹ per pmol nucleic acid-guided complex. In embodiment 15 provided herein is the composition of any previous embodiment wherein the ssODN or ssODNs that are complementary to and specific for a sequence flanking a strand break have a length of at least 100, 120, 140, 160, 180, 200, 220, 240, 260, 280, 300, 350, 400, 450, 500, or 1000 and/or not more than 120, 140, 160, 180, 200, 220, 240, 260, 280, 300, 350, 400, 450, 500, 1000, or 2000 nucleotides, for example 100-500 nucleotides. In embodiment 16 provided herein is the composition of any previous embodiment wherein the nucleic acid-guided nuclease is a Class 1 nuclease. In embodiment 17 provided herein is the composition of any one of embodiments 1 through 15 wherein the nucleic acid-guided nuclease is a Class 2 nuclease. In embodiment 18 provided herein is the composition of embodiment 17 wherein the nucleic acid-guided nuclease is a Type II or a Type V nuclease. In embodiment 19 provided herein is the composition of embodiment 18 wherein the nucleic acid-guided nuclease is a Type V-A, V-B, V-C, V-D, or V-E nuclease. In embodiment 20 provided herein is the composition of embodiment 19 wherein the nucleic acid-guided nuclease is a Type V-A nuclease. In embodiment 21 provided herein is the composition of embodiment 20 wherein the nucleic acid-guided nuclease is a MAD nuclease, an ART nuclease, or an ABW nuclease. In embodiment 22 provided herein is the composition of embodiment 21 wherein the nucleic acid-guided nuclease is a MAD1, MAD2, MAD3, MAD4, MAD5, MAD6, MAD7, MAD8, MAD9, MAD10, MAD1I, MAD12, MAD13, MAD14, MAD15, MAD16, MAD17, MAD18, MAD19, or MAD20 nuclease. In embodiment 23 provided herein is the composition of embodiment 21 wherein the nucleic acid-guided nuclease is an ART1, ART2, ART3, ART4, ART5, ART6, ART7, ART8, ART9, ART10, ART11, ART11*, ART12, ART13, ART14, ART15, ART16, ART17, ART18, ART19, ART20, ART21, ART22, ART23, ART24, ART25, ART26, ART27, ART28, ART29, ART30, ART31, ART32, ART33, ART34, or ART35 nuclease. In embodiment 24 provided herein is the composition of any one of embodiments 20 through 23 wherein the nucleic acid-guided nuclease has an amino acid sequence at least 80, 85, 90, 95, 97, 98, 99, or 100% identical to the amino acid sequence of MAD2, MAD7, ART2, ART11, or ART11*. In embodiment 25 provided herein is the composition of embodiment 19 wherein the nucleic acid-guided nuclease has an amino acid sequence that is at least 80, 85, 90, 95, 99, or 100% identical to the amino acid sequence of SEQ ID NO: 37. In embodiment 26 provided herein is the composition of any previous embodiment wherein the nucleic acid-guided nuclease comprises at least one nuclear localization signal (NLS), at least one purification tag, or at least one cleavage site. In embodiment 27 provided herein is the composition of embodiment 26 wherein the nucleic acid-guided nuclease comprises at least 4 NLS. In embodiment 28 provided herein is the composition of embodiment 27 wherein the nucleic acid-guided nuclease comprises one N-terminal and three C-terminal NLS. In embodiment 29 provided herein is the composition of embodiment 27 wherein the nucleic acid-guided nuclease comprises at least five NLS. In embodiment 30 provided herein is the composition of embodiment 29 wherein the nucleic acid-guided nuclease comprises five N-terminal NLS. In embodiment 31 provided herein is the composition of any one of embodiments 26 through 30 wherein the NLSs comprise any of SEQ ID NOs: 40-56. In embodiment 32 provided herein is the composition of embodiment 31 wherein the NLSs comprise any of SEQ ID NOs: 40. 51, and 56. In embodiment 33 provided herein is the composition of any previous embodiment wherein the gNA comprises (A) a targeter nucleic acid comprising a targeter stem sequence and a spacer sequence; and (B) a modulator nucleic acid comprising a modulator stem sequence complementary to the targeter stem sequence, and. optionally, a 5′ sequence. In embodiment 34 provided herein is the composition of any previous embodiment wherein the gNA is an engineered, non-naturally occurring guide nucleic acid. In embodiment 35 provided herein is the composition of any previous embodiment wherein the gNA comprises a single polynucleotide. In embodiment 36 provided herein is the composition of any one of embodiments 1 through 34 wherein the gNA comprises a dual guide nucleic acid, wherein the targeter nucleic acid and the modulator nucleic acid are separate polynucleotides. In embodiment 37 provided herein is the composition of embodiment 36 wherein the dual gNA is capable of binding to and activating a nucleic acid-guided nuclease, that, in a naturally occurring system, is activated by a single crRNA in the absence of a tracrRNA. In embodiment 38 provided herein is the composition of any previous embodiment wherein the gNA comprises a spacer sequence of any one of SEQ ID NOs: 86-384 and 983-1798. In embodiment 39 provided herein is the composition of any previous embodiment wherein some or all of the gNA is RNA. In embodiment 40 provided herein is the composition of embodiment 39 wherein at least 50%, at least 70%, at least 90%, at least 95%, or 100% of the gNA comprises RNA. In embodiment 41 provided herein is the composition of any previous embodiment wherein the gNA comprises one or more chemical modifications. In embodiment 42 provided herein is the composition of embodiment 41 wherein the chemical modification comprises a 2′-O-alkyl, a 2′-O-methyl, a phosphorothioate, a phosphonoacetate, a thiophosphonoacetate, a 2′-O-methyl-3′-phosphorothioate, a 2′-O-methyl-3′-phosphonoacetate, a 2′-O-methyl-3′-thiophosphonoacetate, a 2′-deoxy-3′-phosphonoacetate, a 2′-deoxy-3′-thiophosphonoacetate, or a combination thereof.

In embodiment 43 provided herein is a kit comprising the composition of any previous embodiment.

In embodiment 44 provided herein is a cell comprising the composition of any one of embodiments 1 through 42. In embodiment 45 provided herein is the cell of embodiment 44 wherein the cell is a human cell. In embodiment 46 provided herein is the cell of embodiment 45 wherein the human cell comprises an immune cell or a stem cell. In embodiment 47 provided herein is the cell of embodiment 46 wherein the immune cell is a neutrophil, eosinophil, basophil, mast cell, monocyte, macrophage, dendritic cell, natural killer cell, or a lymphocyte. In embodiment 48 provided herein is the cell of embodiment 47 wherein the immune cell is a T cell. In embodiment 49 provided herein is the cell of embodiment 48 wherein the immune cell is a CAR-T cell. In embodiment 50 provided herein is the cell of embodiment 46 wherein the stem cell is a human pluripotent, multipotent stem cell, embryonic stem cell, induced pluripotent stem cell, or hematopoietic stem cell. In embodiment 51 provided herein is the cell of embodiment 50 wherein the stem cell is a CD34+ stem cell or an induced pluripotent stem cell (iPSC).

In embodiment 52 provided herein is a method of cleaving at or near a target nucleic acid sequence which is at or near an on-target site within a target polynucleotide comprising contacting the target polynucleotide with the composition of any one of embodiments 2 through 42, wherein the nucleic acid-guided nuclease complex cleaves at least one strand of the target polynucleotide within the on-target site.

In embodiment 53 provided herein is a method of editing a genome of a eukaryotic cell comprising delivering the composition of any one of embodiments 2 through 42 into the eukaryotic cell, thereby resulting in editing of the genome of the eukaryotic cell. In embodiment 54 provided herein is the method of embodiment 53, wherein the composition is delivered by electroporation.

In embodiment 55 provided herein is a method of treating a disease or a disorder comprising administering to a subject in need thereof an effective amount of the composition of any one of embodiments 2 through 42, or an effective amount of cells modified by treatment with a composition of any one of embodiments 2 through 42.

In embodiment 56 provided herein is a method of reducing the proportion of mutations in off-target sites in a genome of a cell comprising contacting the cell with the composition any one of embodiments 2 through 42, compared to the proportion if the composition is not used. In embodiment 57 provided herein is the method of embodiment 56 also comprising increasing homology-directed repair (HDR). In embodiment 58 provided herein is the method of embodiment 56 also comprising increasing viability and/or expansion capacity of cells after editing.

In embodiment 59 provided herein is a method of both increasing HDR at an on-target site in a genome of a cell and decreasing mutations at one or more off-target sites in the genome of the cell comprising contacting the cell with a composition of any one of embodiments 2 through 42, thereby both increasing HDR at the on-target site and decreasing the proportion of mutations in off-target sites of the genome of the cell compared to the proportion if the composition is not used.

In embodiment 60 provided herein is a composition comprising (A) a nucleic acid-guided nuclease complex comprising a Type V nuclease and a compatible gNA wherein the nucleic acid-guided nuclease complex specifically binds to a target nucleic acid sequence at or near an on-target site and cleaves at or near the target nucleic acid sequence to create a strand break in the on-target site; and (B) a first ssODN. In embodiment 61 provided herein is the composition of embodiment 60 wherein the first ssODN comprises a sequence that is complementary to a sequence flanking the strand break in the on-target site on the 3′ side of the strand break. In embodiment 62 provided herein is the composition of embodiment 60 wherein the first ssODN comprises a sequence that is complementary to a sequence flanking the strand break in the on-target site on the 5′ side of the strand break. In embodiment 63 provided herein is the composition of embodiment 61 further comprising a second ssODN comprising a sequence that is complementary to a sequence flanking the strand break in the on-target site on the 5′ side of the strand break. In embodiment 64 provided herein is the composition of embodiment 62 further comprising a second ssODN comprising a sequence that is complementary to a sequence flanking the strand break in the on-target site on the 3′ side of the strand break. In embodiment 65 provided herein is the composition of embodiment 63 or embodiment 64 wherein the first and second ssODNs are the same. In embodiment 66 provided herein is the composition of embodiment 63 or embodiment 64 wherein the first and second ssODNs are different. In embodiment 67 provided herein is the composition of any one of embodiments 60 through 66 wherein at least a portion of the first and/or second ssODNs are capable of being integrated at or near the strand break. In embodiment 68 provided herein is the composition of any one of embodiments 60 through 67 further comprising a donor template separate from ssODNs. In embodiment 69 provided herein is the composition of any one of embodiments 60 through 68 wherein the nucleic acid-guided nuclease complex also binds to one or more off-target nucleic acid sequences at or near one or more off-target sites and cleaves at or near the one or more off-target nucleic acid sequences to create a strand break in the one or more off-target sites. In embodiment 70 provided herein is the composition of embodiment 69 further comprising one or more ssODNs that are complementary to a sequence flanking the strand break in the one or more off-target sites. In embodiment 71 provided herein is the composition of embodiment 70 comprising a plurality of ssODNs each of which comprises a different sequence complementary to sequences flanking the strand break in the different off-target sites. In embodiment 72 provided herein is the composition of embodiment 71 comprising at least 1, 2, 3, 4, 5, 6, 7, 8, 9, 10, 20, 30, 40, 50, 60, 70, 80, 90, 100, 200, 300, 400, 500, 700, or 1000 and/or no more than 2, 3, 4, 5, 6, 7, 8, 9, 10, 20, 30, 40, 50, 60, 70, 80, 90, 100, 200, 300, 400, 500, 700, 1000 or 2000 ssODNs each of which comprises a different sequence complementary to sequences flanking the strand break in the different off-target sites. In embodiment 73 provided herein is the composition of any one of embodiments 70 through 72 wherein one or more of the ssODNs comprising sequences complementary to a sequence flanking the double stranded break at the one or more off-target sites comprise a mutation in the PAM. In embodiment 74 provided herein is the composition of any one of embodiments 60 through 73 wherein the nucleic acid-guided nuclease is a Class 1 nuclease. In embodiment 75 provided herein is the composition of any one of embodiments 60 through 73 wherein the nucleic acid-guided nuclease is a Class 2 nuclease. In embodiment 76 provided herein is the composition of embodiment 75 wherein the nucleic acid-guided nuclease is a Type II or a Type V nuclease. In embodiment 77 provided herein is the composition of embodiment 76 wherein the nucleic acid-guided nuclease is a Type V-A, V-B, V-C, V-D, or V-E nuclease. In embodiment 78 provided herein is the composition of embodiment 77 wherein the nuclease is a Type V-A nuclease. In embodiment 79 provided herein is the composition of embodiment 77 wherein the nucleic acid-guided nuclease is a MAD nuclease, an ART nuclease, or an ABW nuclease. In embodiment 80 provided herein is the composition of embodiment 77 wherein the nucleic acid-guided nuclease is a MAD1, MAD2, MAD3, MAD4, MAD5, MAD6, MAD7, MAD8, MAD9, MAD10, MAD1I, MAD12, MAD13, MAD14, MAD15, MAD16, MAD17, MAD18, MAD19, or MAD20 nuclease. In embodiment 81 provided herein is the composition of embodiment 77 wherein the nucleic acid-guided nuclease is an ART1, ART2, ART3, ART4, ART5, ART6, ART7, ART8, ART9, ART10, ART11, ART11*, ART12, ART13, ART14, ART15, ART16, ART17, ART18, ART19, ART20, ART21, ART22, ART23, ART24, ART25, ART26, ART27, ART28, ART29, ART30, ART31, ART32, ART33, ART34, or ART35 nuclease. In embodiment 82 provided herein is the composition of embodiment 77 wherein the nucleic acid-guided nuclease has an amino acid sequence at least 80, 85, 90, 95, 99, or 100% % identical to the amino acid sequence of MAD2, MAD7, ART2, ART11, or ART11*. In embodiment 83 provided herein is the composition of embodiment 77, wherein the nucleic acid-guided nuclease comprises an amino acid sequence that is at least 80, 85, 90, 95, 99, or 100% identical to the amino acid sequence of SEQ ID NO: 37. In embodiment 84 provided herein is the composition of any one of embodiments 60 through 83 wherein the nucleic acid-guided nuclease comprises at least one nuclear localization signal (NLS), at least one purification tag, or at least one cleavage site. In embodiment 85 provided herein is the composition of embodiment 84 wherein the nucleic acid-guided nuclease comprises at least 4 NLSs. In embodiment 86 provided herein is the composition of embodiment 85 wherein the nucleic acid-guided nuclease comprises one N-terminal and three C-terminal NLS. In embodiment 87 provided herein is the composition of embodiment 85 wherein the nucleic acid-guided nuclease comprises at least five NLS. In embodiment 88 provided herein is the composition of embodiment 87 wherein the nucleic acid-guided nuclease comprises five N-terminal NLS. In embodiment 89 provided herein is the composition of any one of embodiments 84 through 88 wherein the NLSs comprise any of SEQ ID NOs: 40-56. In embodiment 90 provided herein is the composition of embodiment 89 wherein the NLSs comprises any of SEQ ID NOs: 40. 51, and 56. In embodiment 91 provided herein is the composition of any one of embodiments 60 through 90 wherein the gNA comprises (A) a targeter nucleic acid comprising a targeter stem sequence and a spacer sequence; and (B) a modulator nucleic acid comprising a modulator stem sequence complementary to the targeter stem sequence, and, optionally, a 5′ sequence. In embodiment 92 provided herein is the composition of any one of embodiments any one of embodiments 60 through 91 wherein the gNA is an engineered, non-naturally occurring guide nucleic acid. In embodiment 93 provided herein is the composition of any one of embodiments any one of embodiments 60 through 92 wherein the gNA comprises a single polynucleotide. In embodiment 94 provided herein is the composition of any one of embodiments any one of embodiments 60 through 92 wherein the gNA comprises a dual guide nucleic acid, wherein the targeter nucleic acid and the modulator nucleic acid are separate polynucleotides. In embodiment 95 provided herein is the composition of embodiment 94 wherein the dual gNA is capable of binding to and activating a nucleic acid-guided nuclease. that, in a naturally occurring system, is activated by a single crRNA in the absence of a tracrRNA. In embodiment 96 provided herein is the composition of any one of embodiments 60 through 95 wherein the gNA comprises a spacer sequence of any one of SEQ ID NOs: 86-384 and 983-1798. In embodiment 97 provided herein is the composition of any one of embodiments 60 through 96 wherein some or all of the gNA is RNA. In embodiment 98 provided herein is the composition of embodiment 97 wherein at least 50%, at least 70%, at least 90%, at least 95%, or 100% of the gNA comprises RNA. In embodiment 99 provided herein is the composition of any one of embodiments 60 through 98 wherein the gNA comprises one or more chemical modifications. In embodiment 100 provided herein is the composition of embodiment 99 wherein the chemical modification comprises a 2′-O-alkyl, a 2′-O-methyl, a phosphorothioate, a phosphonoacetate, a thiophosphonoacetate, a 2′-O-methyl-3′-phosphorothioate, a 2′-O-methyl-3′-phosphonoacetate, a 2′-O-methyl-3′-thiophosphonoacetate, a 2′-deoxy-3′-phosphonoacetate, a 2′-deoxy-3′-thiophosphonoacetate or a combination thereof. In embodiment 101 provided herein is the composition of any one of embodiments 60 through 100 wherein the ssODN is at least 100, 120, 140, 160, 180, 200, 220, 240, 260, 280, 300, 350, 400, 450, 500, or 1000 and/or not more than 120, 140, 160, 180, 200, 220, 240, 260, 280, 300, 350, 400, 450, 500, 1000, or 2000 nucleotides, for example 100-500 nucleotides. In embodiment 102 provided herein is the composition of any one of embodiments 60 through 101 comprising at least 50, 60, 70, 80, 90, 100, 150, 200, 250, 300, 350, 400, 450, 500, 550, 600, 700, 800, or 900 and/or not more than 60, 70, 80, 90, 100, 150, 200, 250, 300, 350, 400, 450, 500, 550, 600, 700, 800, 900 or 1000 pmol of each ssODN, for example 50-1000 pmol of each ssODN. In embodiment 103 provided herein is the composition of any one of embodiments 60 through 102 further comprising a HDR enhancer. In embodiment 104 provided herein is the composition of embodiment 103 wherein the HDR enhancer comprises M3814. In embodiment 105 provided herein is the composition of embodiment 104 wherein the M3814 concentration is at least 0.1, 0.25, 0.5, 0.75, 1, 1.25, 1.5, 1.75, 2, 2.5, 3, or 4 and/or not more than 0.25, 0.5, 0.75, 1, 1.25, 1.5, 1.75, 2, 2.5, 3, 4, or 5 μM, for example 0.1-5 μM. In embodiment 106 provided herein is the composition of any one of embodiments 60 through 105 further comprising an anionic polymer. In embodiment 107 provided herein is the composition of embodiment 106 wherein the anionic polymer comprises a non-specific ssODN or a peptide, or poly-L-glutamic acid (PGA). In embodiment 108 provided herein is the composition of embodiment 107 wherein the peptide comprises poly-L-glutamic acid (PGA). In embodiment 109 provided herein is the composition of embodiment 107 comprising at least 50, 60, 70, 80, 90, 100, 150, 200, 250, 300, 350, 400, 450, 500, 550, 600, 700, 800, or 900 and/or not more than 60, 70, 80, 90, 100, 150, 200, 250, 300, 350, 400, 450, 500, 550, 600, 700, 800, 900 or 1000 pmol non-specific ssODN. In embodiment 110 provided herein is the composition of embodiment 108 wherein the PGA is present at a concentration of at least 0.01, 0.05. 0.1, 0.15, 0.2, 0.25, 0.3, 0.35, 0.4, 0.45, 0.5, 0.55, 0.6, 0.65, 0.7, 0.75, 0.8, 0.85, 0.9. 0.95, 1, 1.5, 2, 2.5, 3, 3.5, 4, or 4.5 and/or not more than 0.05, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.5, 2, 2.5, 3, 3.5, 4, 4.5, or 5 μg μL⁻¹ per pmol RNP complex, for example 0.01-5 μg μL⁻¹ per pmol RNP complex.

In embodiment 111 provided herein is a cell comprising the composition of any one of embodiments 60 through 110. In embodiment 112 provided herein is the cell of embodiment 111, wherein the cell is a human cell. In embodiment 113 provided herein is the cell of embodiment 112 wherein the human cell is an immune cell or a stem cell. In embodiment 114 provided herein is the cell of embodiment 113 wherein the immune cell is a neutrophil, cosinophil, basophil, mast cell, monocyte, macrophage, dendritic cell, natural killer cell, or a lymphocyte. In embodiment 115 provided herein is the cell of embodiment 114 wherein the immune cell is a T cell. In embodiment 116 provided herein is the cell of embodiment 115 wherein the immune cell is a CAR-T cell. In embodiment 117 provided herein is the cell of embodiment 113 wherein the stem cell is a human pluripotent, multipotent stem cell, embryonic stem cell, induced pluripotent stem cell, or hematopoietic stem cell. In embodiment 118 provided herein is the cell of embodiment 117 wherein the stem cell is a CD34+ stem cell or an iPSC.

In embodiment 119 provided herein is a composition comprising (A) a first ssODN; and (B) a HDR enhancer. In embodiment 120 provided herein is the composition of embodiment 119, wherein the first ssODN comprises a sequence complementary to a sequence flanking a double stranded break at an on-target site. In embodiment 121 provided herein is the composition of embodiment 119 or embodiment 120, further comprising (C) nucleic acid-guided nuclease complex comprising a Type V nucleic acid-guided nuclease and a compatible gNA, wherein the nucleic acid-guided nuclease complex specifically binds to a target nucleic acid sequence at or near an on-target site and cleaves at or near the target nucleic acid sequence to create a double-stranded break in the on-target site. In embodiment 122 provided herein is the composition of embodiment 121 wherein the nucleic acid-guided nuclease complex also binds to one or more off-target nucleic acid sequences at one or more off-target sites and cleaves at or near the one or more off-target nucleic acid sequences to create on or more double-strand breaks at the one or more off-target sites. In embodiment 123 provided herein is the composition of embodiment 122 further comprising a ssODN comprising a sequence complementary to a sequence flanking a double stranded break at an off-target site In embodiment 124 provided herein is the composition of embodiment 123 comprising a plurality of ssODNs each of which comprises a different sequence complementary to a sequence flanking a double stranded break at different off-target sites. In embodiment 125 provided herein is the composition of embodiment 124 comprising at least 1, 2, 3, 4, 5, 6, 7, 8, 9, 10, 20, 30, 40, 50, 60, 70, 80, 90, 100, 200, 300, 400, 500, 700, or 1000 and/or no more than 2, 3, 4, 5, 6, 7, 8, 9, 10, 20, 30, 40, 50, 60, 70, 80, 90, 100, 200, 300, 400, 500, 700, 1000 or 2000 ssODNs each of which comprises a different sequence complementary to a sequence flanking a double stranded break at different off-target sites. In embodiment 126 provided herein is the composition of any one of embodiments 123 through 125 wherein one or more of the ssODNs complementary to a sequence flanking the double stranded break at the one or more off-target sites comprise a mutation in the PAM. In embodiment 127 provided herein is the composition of any one of embodiments 121 through 126 wherein the nucleic acid-guided nuclease is a Class 1 nuclease. In embodiment 128 provided herein is the composition any one of embodiments 121 through 126 wherein the nucleic acid-guided nuclease is a Class 2 nuclease. In embodiment 129 provided herein is the composition of embodiment 128 wherein the nucleic acid-guided nuclease is a Type II or a Type V nuclease. In embodiment 130 provided herein is the composition of embodiment 129 wherein the nucleic acid-guided nuclease is a Type V-A, V-B, V-C, V-D, or V-E nuclease. In embodiment 131 provided herein is the composition of embodiment 130 wherein the nuclease is a Type V-A nuclease. In embodiment 132 provided herein is the composition of embodiment 131 wherein the nucleic acid-guided nuclease is a MAD nuclease, an ART nuclease, or an ABW nuclease. In embodiment 133 provided herein is the composition of embodiment 132 wherein the nucleic acid-guided nuclease comprises a MAD1, MAD2, MAD3, MAD4, MAD5, MAD6, MAD7, MAD8, MAD9, MAD10, MAD11, MAD12, MAD13, MAD14, MAD15, MAD16, MAD17, MAD18, MAD19, or MAD20 nuclease. In embodiment 134 provided herein is the composition of embodiment 132 wherein the nucleic acid-guided nuclease is an ART1, ART2, ART3, ART4, ART5, ART6, ART7, ART8, ART9, ART10, ART11, ART11*, ART12, ART13, ART14, ART15, ART16, ART17, ART18, ART19, ART20, ART21, ART22, ART23, ART24, ART25, ART26, ART27, ART28, ART29, ART30, ART31, ART32, ART33, ART34, or ART35 nuclease. In embodiment 135 provided herein is the composition of embodiment 132 wherein the nucleic acid-guided nuclease comprises an amino acid sequence at least 80% identical to the amino acid sequence of MAD2, MAD7, ART2, ART11, or ART11*. In embodiment 136 provided herein is the composition of embodiment 132 wherein the nucleic acid-guided nuclease comprises an amino acid sequence that is at least 80, 85, 90, 95, 99, or 100% identical to the amino acid sequence of SEQ ID NO: 37. In embodiment 137 provided herein is the composition of any one of embodiments 121 through 136 wherein the nucleic acid-guided nuclease comprises at least one nuclear localization signal (NLS), at least one purification tag, or at least one cleavage site. In embodiment 138 provided herein is the composition of embodiment 137 wherein the nucleic acid-guided nuclease comprises at least 4 NLS. In embodiment 139 provided herein is the composition of embodiment 138 wherein the nucleic acid-guided nuclease comprises one N-terminal and three C-terminal NLS. In embodiment 140 provided herein is the composition of embodiment 138 wherein the nucleic acid-guided nuclease comprises at least five NLS. In embodiment 141 provided herein is the composition of embodiment 140 wherein the nucleic acid-guided nuclease comprises five N-terminal NLS. In embodiment 142 provided herein is the composition of any one of embodiments 137 through 141 wherein the NLSs comprise any of SEQ ID NOs: 40-56. In embodiment 143 provided herein is the composition of embodiment 142 wherein the NLSs comprises any of SEQ ID NOs: 40, 51, and 56. In embodiment 144 provided herein is the composition of any one of embodiments 121 through 143 wherein the nucleic acid-guided nuclease complex comprises a guide nucleic acid (gNA). In embodiment 145 provided herein is the composition of embodiment 144 wherein the gNA comprises: (A) a targeter nucleic acid comprising a targeter stem sequence and a spacer sequence; and (B) a modulator nucleic acid comprising a modulator stem sequence complementary to the targeter stem sequence, and, optionally, a 5′ sequence. In embodiment 146 provided herein is the composition of any one of embodiments 144 or embodiment 145 wherein the gNA an engineered, non-naturally occurring guide nucleic acid. In embodiment 147 provided herein is the composition of any one of embodiments 144 through 146 wherein the gNA comprises a single polynucleotide. In embodiment 148 provided herein is the composition of any one of embodiments 144 through 146 wherein the gNA comprises a dual guide nucleic acid, wherein the targeter nucleic acid and the modulator nucleic acid are separate polynucleotides. In embodiment 149 provided herein is the composition of embodiment 148 wherein the dual gNA is capable of binding to and activating a nucleic acid-guided nuclease, that, in a naturally occurring system, is activated by a single crRNA in the absence of a tracrRNA. In embodiment 150 provided herein is the composition of any one of embodiments 144 through 149 wherein the gNA comprises a spacer sequence of any one of SEQ ID NOs: 86-384 and 983-1798. In embodiment 151 provided herein is the composition of any one of embodiments 144 through 150 wherein some or all of the gNA is RNA. In embodiment 152 provided herein is the composition of embodiment 151 wherein at least 50%, at least 70%, at least 90%, at least 95%, or 100% of the gNA comprises RNA. In embodiment 153 provided herein is the composition of any one of embodiments 144 through 152 wherein the gNA comprises one or more chemical modifications. In embodiment 154 provided herein is the composition of embodiment 153 wherein the chemical modification comprises a 2′-O-alkyl, a 2′-O-methyl, a phosphorothioate, a phosphonoacetate, a thiophosphonoacetate, a 2′-O-methyl-3′-phosphorothioate, a 2′-O-methyl-3′-phosphonoacetate, a 2′-O-methyl-3′-thiophosphonoacetate, a 2′-deoxy-3′-phosphonoacetate, a 2′-deoxy-3′-thiophosphonoacetate or a combination thereof. In embodiment 155 provided herein is the composition of any one of embodiments 119 through 154 wherein the ssODN or ssODNS have a length of at least 100, 120, 140, 160, 180, 200, 220, 240, 260, 280, 300, 350, 400, 450, 500, or 1000 and/or not more than 120, 140, 160, 180, 200, 220, 240, 260, 280, 300, 350, 400, 450. 500, 1000, or 2000 nucleotides, for example 100-500 nucleotides. In embodiment 156 provided herein is the composition of any one of embodiments 119 through 155 comprising at least 50, 60, 70, 80, 90, 100, 150, 200, 250, 300, 350, 400, 450, 500, 550, 600, 700, 800, or 900 and/or not more than 60, 70, 80, 90, 100, 150, 200, 250, 300, 350, 400, 450, 500, 550, 600, 700, 800, 900 or 1000 pmol of each ssODN, for example 50-1000 pmol of each ssODN. In embodiment 157 provided herein is the composition of any one of embodiments 119 through 156 wherein the HDR enhancer comprises M3814. In embodiment 158 provided herein is the composition of embodiment 157 wherein the M3814 is present at a concentration of at least 0.1, 0.25, 0.5, 0.75, 1, 1.25, 1.5, 1.75, 2, 2.5, 3, or 4 and/or not more than 0.25, 0.5, 0.75, 1, 1.25, 1.5, 1.75, 2, 2.5, 3, 4, or 5 μM, for example 0.1-5 μM. In embodiment 159 provided herein is the composition of any one of embodiments 119 through 158 further comprising an anionic polymer. In embodiment 160 provided herein is the composition of embodiment 159, wherein the anionic polymer comprises a non-specific ssODN or a peptide. In embodiment 161 provided herein is the composition of embodiment 160 comprising a peptide. In embodiment 162 provided herein is the composition of embodiment 161 wherein the peptide comprises poly-L-glutamic acid (PGA). In embodiment 163 provided herein is the composition of embodiment 160 comprising at least 50, 60, 70, 80, 90, 100, 150, 200, 250, 300, 350, 400, 450, 500, 550, 600, 700, 800, or 900 and/or not more than 60, 70, 80, 90, 100, 150, 200, 250, 300, 350, 400, 450, 500, 550, 600, 700, 800, 900 or 1000 pmol non-specific ssODN. In embodiment 164 provided herein is the composition of embodiment 162 wherein the PGA is present at a concentration of at least 0.01, 0.05, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.5, 2, 2.5, 3, 3.5, 4, or 4.5 and/or not more than 0.05, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.5, 2, 2.5, 3, 3.5, 4, 4.5, or 5 μg μL⁻¹ per pmol RNP complex, for example 0.01-5 μg μL⁻¹ per pmol RNP complex.

In embodiment 165 provided herein is a cell comprising the composition of any one of embodiments 119 through 164. In embodiment 166 provided herein is the cell of embodiment 165 wherein the cell is a human cell. In embodiment 167 provided herein is the cell of embodiment 166 wherein the human cell is an immune cell or a stem cell. In embodiment 168 provided herein is the cell of embodiment 167 wherein the immune cell is a neutrophil, cosinophil, basophil, mast cell, monocyte, macrophage, dendritic cell, natural killer cell, or a lymphocyte. In embodiment 169 provided herein is the cell of embodiment 168 wherein the immune cell is a T cell. In embodiment 170 provided herein is the cell of embodiment 169 wherein the immune cell is a CAR-T cell. In embodiment 171 provided herein is the cell of embodiment 167 wherein the stem cell is a human pluripotent, multipotent stem cell, embryonic stem cell, induced pluripotent stem cell, or hematopoietic stem cell. In embodiment 172 provided herein is the cell of embodiment 171 wherein the stem cell is a CD34+ stem cell or an iPSC.

In embodiment 173 provided herein is a composition comprising (A) a ssODN comprising a sequence complementary to a nucleic acid sequence flanking a double stranded break at an on-target site for a nucleic acid-guided nuclease complex; and (B) a ssODN comprising a sequence complementary to a nucleic acid sequence flanking a double stranded break at an off-target site (ssODNoff) for the nucleic acid-guided nuclease complex. In embodiment 174 provided herein is the composition of embodiment 173 further comprising, for each integer x representing an off-target site for the nucleic-acid guided nuclease complex, a (ssODNoff)x wherein each (ssODNoff)x comprises a sequence complementary to a nucleic acid sequence flanking a double stranded break at an off-target site (x). In embodiment 175 provided herein is the composition of embodiment 174 wherein the number of different integers x is at least 2, 3, 4, 5, 6, 7, 8, 9, 10, 20, 30, 40, 50, 60, 70, 80, 90, 100, 120, 140, 160, 180, 200, 250, 300, 350, 400, 450, 500, or 1000 and/or no more than 3, 4, 5, 6, 7, 8, 9, 10, 20, 30, 40, 50, 60, 70, 80, 90, 100, 120, 140, 160, 180, 200, 250, 300, 350, 400, 450, 500, 1000, or 2000. In embodiment 176 provided herein is the composition of embodiment 175 where the number of different integers x is 2-2000. In embodiment 177 provided herein is the composition of embodiment 175 wherein the number of different integers x is 2-1000. In embodiment 178 provided herein is the composition of any one of embodiments 173 through 177 wherein the ssODN comprising a sequence complementary to a nucleic acid sequence flanking a double stranded break at an on-target site comprises at least one mutation compared to the wildtype sequence at the on-target site. In embodiment 179 provided herein is the composition of embodiment 178 wherein the mutation comprises a SNP, an INDEL, and/or a missense mutation. In embodiment 180 provided herein is the composition of any one of embodiments 173 through 179 wherein the ssODN or ssODNs comprising a sequence complementary to a nucleic acid sequence flanking a double stranded break at one or more off-target sites comprises the wildtype sequence for the one or more off-target sites. In embodiment 181 provided herein is the composition of any one of embodiments 173 through 180 wherein the ssODN or ssODNS comprising a sequence complementary to a nucleic acid sequence flanking a double stranded break at one or more off-target sites comprises at least one mutation compared to the wildtype sequence at the one or more off-target sites. In embodiment 182 provided herein is the composition of embodiment 181 wherein the mutation comprises a synonymous mutation. In embodiment 183 provided herein is the composition of embodiment 181 or embodiment 182, wherein the mutation is in the PAM at the one or more off-target sites.

In embodiment 184 provided herein is a method comprising delivering the composition of any one of embodiments 121 through 183 to a population of cells. In embodiment 185 provided herein is the method of embodiment 184 further comprising expanding and/or differentiating cells in the population of cells. In embodiment 186 provided herein is the method of embodiment 184 or embodiment 185 further comprising adding a HDR enhancer to the growth medium prior to expanding and/or differentiating cells in the population of cells. In embodiment 187 provided herein is the method of embodiment 186 wherein the HDR enhancer comprises M3814. In embodiment 188 provided herein is the method of embodiment 187 wherein the M3814 concentration is at least 0.1, 0.25, 0.5, 0.75, 1, 1.25, 1.5, 1.75, 2, 2.5, 3, or 4 and/or not more than 0.25, 0.5, 0.75, 1, 1.25, 1.5, 1.75, 2, 2.5, 3, 4, or 5 μM, for example 0.1-5 μM. In embodiment 189 provided herein is the method of any one of embodiments 184 through 188 further comprising, before delivering the composition, combining the nucleic acid-guided nuclease complex with an anionic polymer. In embodiment 190 provided herein is the method of embodiment 189 wherein the anionic polymer comprises a non-specific ssODN or a peptide. In embodiment 191 provided herein is the method of embodiment 190 wherein the peptide comprises poly-L-glutamic acid (PGA). In embodiment 192 provided herein is the method of embodiment 190 wherein the composition comprises at least 50, 60, 70, 80, 90, 100, 150, 200, 250, 300, 350, 400, 450, 500, 550, 600, 700, 800, or 900 and/or not more than 60, 70, 80, 90, 100, 150, 200, 250, 300, 350, 400, 450, 500, 550, 600, 700, 800, 900 or 1000 pmol non-specific ssODN. In embodiment 193 provided herein is the method of embodiment 190 wherein the PGA is present at a concentration of at least 0.01, 0.05, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.5, 2, 2.5, 3, 3.5, 4, or 4.5 and/or not more than 0.05, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.5, 2, 2.5, 3, 3.5, 4, 4.5, or 5 μg μL⁻¹ per pmol RNP complex, for example 0.01-5 μg μL⁻¹ per pmol RNP complex. In embodiment 194 provided herein is the method of any one of embodiments 184 through 193 wherein the method produces a population of cells comprising a plurality of genotypes at the on-target site. In embodiment 195 provided herein is the method of any one of embodiments 184 through 194 wherein delivering comprises electroporation. In embodiment 196 provided herein is the method of any one of embodiments 184 through 195 wherein, after delivery, one or more cells in the population of cells are (A) expanded: (B) differentiated and then expanded: or (C) expanded, differentiated, and then expanded.

In embodiment 197 provided herein is a method comprising delivering a composition to a cell, wherein the composition comprises (A) a Type V nucleic acid-guided nuclease and a compatible gNA, or one or more polynucleotides encoding the nuclease and/or the gNA, and (B) a ssODN. In embodiment 198 provided herein is the method of embodiment 197 further comprising expanding and/or differentiating the cell.

In embodiment 199 provided herein is a composition for integrating at least a portion of a donor template at or near a strand break at an on-target or off-target site in a genome of a cell comprising (A) a donor template lacking one or both homology arms complementary to a sequence or sequences flanking the strand break; and (B) a first ssODN comprising (i) a first portion comprising a sequence complementary to at least a 5′ or 3′ portion of the donor template, and (ii) a second portion comprising a sequence homologous to a sequence flanking the strand break. In embodiment 200 provided herein is the composition of embodiment 199 further comprising: (C) a second ssODN comprising (i) a first portion comprising a sequence complementary to at least a 5′ or 3′ portion of the donor template different from the first ssODN, and (ii) a second portion comprising a sequence homologous to a sequence flanking the strand break.

In embodiment 201 provided herein is a method for integrating at least a portion of a donor template at a strand break in a target site in a genome of a cell comprising delivering to a cell a composition comprising (A) a composition of any one of embodiments 199 or 200 to the target cell; and (B) a nucleic acid guided nuclease complex comprising a nucleic acid-guided nuclease and a compatible gNA, wherein the complex is capable of producing the strand break. In embodiment 202 provided herein is the method of embodiment 201 further comprising expanding and/or differentiating the cell.

In embodiment 203 provided herein is a composition comprising a plurality of ssODNs comprising (A) a first ssODN comprising (i) a first portion comprising a sequence homologous to a sequence upstream of a target site in a genome of a target cell, and (ii) a second portion comprising a sequence comprising at least a portion of a heterologous sequence to be inserted into the genome of the target cell; (B) a second ssODN comprising (i) a first portion comprising a sequence homologous to a sequence downstream of a target site in a genome of a target cell, and (ii) a second portion comprising a sequence at least partially complementary to at least a portion of the heterologous sequence to be inserted into the genome of the target cell; and, optionally, (C) one or more additional ssODNs each comprising (i) a sequence comprising at least a portion of a heterologous sequence to be inserted into the genome of the target cell, and (ii) a second portion comprising a sequence at least partially complementary to at least a portion of the heterologous sequence to be inserted into the genome of the target cell; wherein the plurality of ssODNs comprises the entirety of heterologous sequence to be inserted into the genome of the target cell.

In embodiment 204 provided herein is a method for inserting a heterologous sequence at or near a target site in a genome of a cell comprising delivering the composition of embodiment 203 to the cell and a nucleic acid-guided nuclease complex capable of binding to and cleaving at the target site. In embodiment 205 provided herein is the method of embodiment 204 further comprising expanding and/or differentiating the cell.

In embodiment 206 provided herein is a method comprising contacting a population of cells with a composition comprising (A) a nucleic acid-guided nuclease complex comprising a nucleic acid-guided nuclease and a compatible gNA, wherein the complex can bind to and cleave at an on-target site and one or more off-target sites in the genomes of the cells in the population of cells, (B) a ssODN, and (C) one or more ssODNs for one or more of the off-target sites. In embodiment 207 provided herein is the method of embodiment 206 further comprising expanding and/or differentiating cells in the population of cells. In embodiment 208 provided herein is the method of any one of embodiments 206 or 207 wherein at least 20% of total genomic edits at the target site occurs through HDR. In embodiment 209 provided herein is the method of any one of embodiments 206 through 208 wherein a mutation rate at the one or more off-target sites is at least 20% lower than that of the same population of cells treated with the composition of embodiment 206 lacking (iii). In embodiment 210 provided herein is the method of embodiment any one of embodiments 206 through 209 further comprising adding a HDR enhancer to the growth medium prior to expanding and/or differentiating.

In embodiment 211 provided herein is a composition comprising (A) a guide RNA (gRNA) comprising (i) a first nucleotide sequence that hybridizes to a target nucleic acid sequence in a genome of a cell, and (ii) a second nucleotide sequence that interacts with a Cas nuclease; (B) the Cas nuclease, comprising an RNA-binding portion that interacts with the second nucleotide sequence of the guide RNA to form a ribonucleoprotein (RNP) complex, wherein the RNP complex (i) specifically binds to the target nucleic acid sequence at an on-target site and cleaves at or near the target nucleic acid sequence to create a double-stranded break in the on-target site, and (ii) also binds to one or more off-target nucleic acid sequences at one or more off-target sites and cleaves at or near the one or more off-target nucleic acid sequences to create a double-strand break in the one or more off-target sites; (C) a first, on-target ssODN comprising a sequence complementary to a sequence flanking the double stranded break in the on-target site, wherein the ssODN integrates into DNA in the on-target site; and (D) a second, off-target ssODN comprising a sequence complementary to a genomic sequence flanking a double stranded break in a first off-target site and integrates into the DNA in the off-target site, wherein the second ssODN comprises (i) homology arms for the off-target site that are more complementary to the genomic sequence at the off-target site than homology arms of the on-target ssODN. In embodiment 212 provided herein is the composition of embodiment 211 wherein the first ssODN comprises at least one nucleotide modification relative to nucleic acid sequence at the on-target site. In embodiment 213 provided herein is the composition of embodiment 211 wherein the second ssODN further comprises at least one synonymous mutation to reduce or eliminate re-cleavage at the off-target site following integration of the second ssODN. In embodiment 214 provided herein is the composition of embodiment 213 wherein the mutation is in a PAM sequence of the first off-target site. In embodiment 215 provided herein is the composition of embodiment 211 further comprising (ii) a nucleotide sequence to be inserted at the off-target site that is identical to a wild-type gene at the first off-target site. In embodiment 216 provided herein is the composition of embodiment 211 further comprising an HDR enhancer. In embodiment 217 provided herein is the composition of embodiment 211 further comprising a third ssODN for a second off-target site. In embodiment 218 provided herein is the composition of embodiment 211 further comprising a fourth ssODN for third off-target site. In embodiment 219 provided herein is the composition of embodiment 211 wherein gRNA is dual gRNA. In embodiment 220 provided herein is the composition of embodiment 211 wherein one or more nucleotides of the gRNA is chemically modified. In embodiment 221 provided herein is the composition of embodiment 211 wherein the nuclease is a Type V nuclease. In embodiment 222 provided herein is the composition of embodiment 221, wherein the Cas nuclease is a type V-A, type V-C, or type V-D Cas nuclease. In embodiment 223 provided herein is the composition of embodiment 222, wherein the Cas nuclease is a type V-A Cas nuclease. In embodiment 224 provided herein is the composition of embodiment 223 wherein the Type V-A Cas nuclease is a Cpf1, MAD, Csm1, ART, or ABW nuclease, or derivative or variant thereof.

### IX. EXAMPLES

### Example 1

In this example, a gRNA was selected for programmed disruption and single-stranded oligos (200 bp) were designed to create a targeted deletion of 25nt (spacer sequence+PAM) thereby creating a modified coding sequence with a frameshift and increasing the likelihood of dysfunctional protein.

The targeted ssODN template (Table 11) was transfected with RNPs in three primary Pan-T donors. Cell pools were harvested post recovery for genotypic evaluation (i.e. modification incorporation via NGS) and also functional analysis (FACS staining). The incorporation of randomized, NHEJ INDELs relative to HDR programmed in all cases show a significant increase in total modified cells. The increase in overall genomic modifications was conserved across a wide range of conditions (Lonza programs) and donors (three donors tested) and implies that the HDR-based approach can be a robust option for disruption optimization for further gene targets.

In addition, FACS analysis for functional disruption was completed in conjunction with genotypic characterization presented above and confirm a significant reduction in functional expression when the ssODN template is present in the RNP transfection for both the single or split (STAR) gRNA configurations. Further, it was observed that results in higher functional knock-out potential when compared with the other top gRNA's for the gene tested and that incorporation of the ssODN template results in complete TCR disruption.

Cell stocks transfected with RNP either with or without the ssODN appear to have slightly reduced viability when compared to the controls that were not transfected with RNP either by not including the buffer or electroporation step at Day 2 (77-86% compared to >95% in the no buffer, spin, and program controls). The impact is reduced by Day 3 (86-90% compared to >90% for the controls) and remains slightly lower throughout the 10-day time course.

Interestingly, inclusion of the ssODN appears to improve viability compared to RNP alone. This implies that the HDR pathway somewhat rescues cells from some of the toxicity effects of NHEJ repair alone.

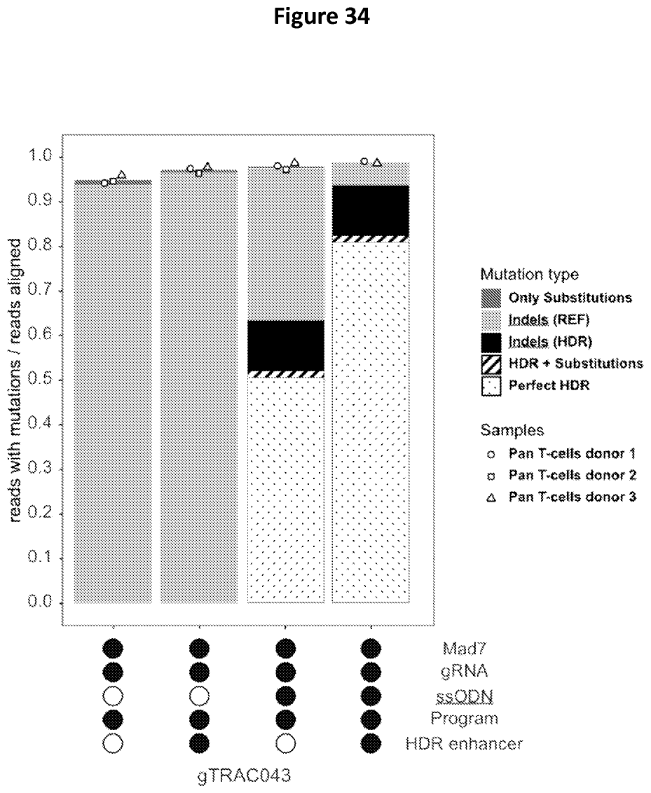

Similar to the observation for viability, transfection with RNP alone (no ssODN) resulted in reduced expansion compared to the no program control and the no buffer control in which no editing occurs. Additionally, the incorporation of the ssODN resulted in expansion similar to that observed for the no buffer and no program controls. Data shown in FIG. 34. Briefly, FIG. 34, shows that inclusion of ssODN dramatically increases perfect HDR.

TABLE 11
exemplary ssODNs

| Name | SEQ ID NO | Sequence |
|---|---|---|
| SDN0001_T | 1799 | GCACAGTTTTGTCTGTGATATACACATCAGAATCCTTACTT |
| RAC43_del | | TGTGACACATTTGTTTGAGAATCAAAATCGGTGAATAGGCA |
| _2 | | GACAGACTTGTCACTGGAGCAGGGTCAGGGTTCTGGATATC |
| | | TGTGGGACAAGAGGATCAGGGTTAGGACATGATCTCATTTC |
| | | CCTCTTTGCCCCAACCCAGGCTGGAGTCCAGATGCC |
| SDN0002_B | 1800 | CAACTTTCAGCAGCTTACAAAAGAATGTAAGACTTACCCCA |
| 2M30_del | | CTTAACTATCTTGGGCTGTGACAAAGTCACATGGTTCACAC |
| | | GGCAGGCATACTCATCTTGTACAAGAGATAGAAAGACCAGT |
| | | CCTTGCTGAAAGACAAGTCTGAATGCTCCACTTTTTCAATT |
| | | CTCTCTCCATTCTTCAGTAAGTCAACTTCAATGTCG |
| SDN0003_C | 1801 | ATCCTCACCCCCATCCCCAATTCAGAATGGTTTCTCTGTTT |
| IITA32_del | | ATCTGGAATGGCAGGACCAGCTGAGACTGCACGCTAAATTA |
| | | AGATGCTTTCCCGGCCTTGACCCAGCAGGGCGTGGAGCCAG |
| | | GCAACGCATTGTGTAGGAATCCCAGCCAGGCAGCAGCTCCC |
| | | GGAGTCTGGCAGCCCCTCCTCGTGCCCTCAGCTTCC |
| SDN000_T | 1802 | GCACAGTTTTGTCTGTGATATACACATCAGAATCCTTACTT |
| RAC43_del | | TGTGACACATTTGTTTGAGAATCAAAATCGGTGAATAGGCA |
| _2_mod | | GACAGACTTGTCACTGGAGCAGGGTCAGGGTTCTGGATATC |
| | | TGTGGGACAAGAGGATCAGGGTTAGGACATGATCTCATTTC |
| | | CCTCTTTGCCCCAACCCAGGCTGGAGTCCAGATGCC |
| SDN0005_T | 1803 | ACCCTGCCGTGTACCAGCTGAGAGACTCTAAATCCAGTGAC |
| RAC049_del | | AAGTCTGTCTGCCTATTCACCGATTTTGATTCTCAAACAAA |
| 2stop_s | | TGTGTCACAAAGTAAGGATTGATAAACTGTGCTAGACATGA |
| | | GGTCTATGGACTTCAAGAGCAACAGTGCTGTGGCCTGGAGC |
| | | AACAAATCTGACTTTGCATGTGCAAACGCCTTCAAC |
| SDN0006_T | 1804 | GTTGAAGGCGTTTGCACATGCAAAGTCAGATTTGTTGCTCC |
| RAC049_del | | AGGCCACAGCACTGTTGCTCTTGAAGTCCATAGACCTCATG |
| 2stop_as | | TCTAGCACAGTTTATCAATCCTTACTTTGTGACACATTTGT |
| | | TTGAGAATCAAAATCGGTGAATAGGCAGACAGACTTGTCAC |
| | | TGGATTTAGAGTCTCTCAGCTGGTACACGGCAGGGT |
| SDN0007_T | 1805 | CGAAGGCACCAAAGCTGCCCTTACCTGGGCIGGGGAAGAAG |
| RAC051_del | | GTGTCTTCTGGAATAATGCTGTTGTTGAAGGCGTTTGCACA |
| 2stop_as | | TGCAAAGTCAGATTATCAGTTGCTCTTGAAGTCCATAGACC |
| | | TCATGTCTAGCACAGTTTTGTCTGTGATATACACATCAGAA |
| | | TCCTTACTTTGTGACACATTTGTTTGAGAATCAAAA |
| T12_g1_TR | 1806 | TCCGTGCTGACCCCACTGTGCACCTCCTTCCCATTCACCCA |
| BC1_del | | CCAGCTCAGCTCCACGTGGTCAGGGAAGAAGCCTGTGGCCA |
| | | GGCACACCAGTGTGGCCTTGATGGCTCAAACACAGCGACCT |
| | | CGGGTGGGAACACCTTGTTCAGGTCCTCTGGAAAGGGAAGA |
| | | GGGGTTGGAGCCAGGGTTGCTCTGAGAGCTGTCTGG |
| T12_g1_TR | 1807 | TCCGTGCTGACCCCACTGTGCACCTCCTTCCCATTCACCCA |
| BC2_del | | CCAGCTCAGCTCCACGTGGTCAGGGAAGAAGCCTGTGGCCA |
| | | GGCACACCAGTGTGGCCTTGATGGCTCAAACACAGCGACCT |
| | | CGGGTGGGAACACCTTGTTCAGGTCCTCTGGAAAGGGAAGA |
| | | GGGGTTGGAGCCAGGGTTGCTCTGAGAGCTGTCTGG |
| T12_g3_TR | 1808 | CCCCTACCAGAACCAGACAGCTCTCAGAGCAACCCIGGCTC |
| BC1_del | | CAACCCCTCTTCCCTTTCCAGAGGACCTGAACAAGGTGTTC |
| | | CCACCCGAGGTCGCTGTGCCCACACCCAAAAGGCCACACTG |
| | | GTGTGCCTGGCCACAGGCTTCTTCCCTGACCACGTGGAGCT |
| | | GAGCTGGTGGGTGAATGGGAAGGAGGTGCACAGTGG |
| T12_g3_TR | 1809 | CCCCTACCAGGACCAGACAGCTCTTAGAGCAACCCTAGCCC |
| BC2_del | | CATTACCTCTTCCCTTTCCAGAGGACCTGAAAAACGTGTTC |
| | | CCACCCAAGGTCGCTGTGCCCACACCCAAAAGGCCACACTG |
| | | GTGTGCCTGGCCACAGGCTTCTACCCCGACCACGTGGAGCT |
| | | GAGCTGGTGGGTGAATGGGAAGGAGGTGCACAGTGG |
| CSF2_g3_ | 1810 | ACACTGCTGCTGAGATGGTAAGTGAGAGAATGTGGGCCTGT |
| del | | GCCTAGGCCACCCAGCTGGCCCCTGACTGGCCACGCCTGTC |
| | | AGCTTGATAACATGACATTAGAAGTCATCTCAGAAATGTTT |
| | | GACCTCCAGGTAAGATGCTTCTCTCTGACATAGCTTTCCAG |
| | | AAGCCCCTGCCCTGGGGTGGAGGTGGGGACTCCATT |
| CSF2_g5_ | 1811 | CTGCTGAGATGGTAAGTGAGAGAATGTGGGCCTGTGCCTAG |
| del | | GCCACCCAGCTGGCCCCTGACTGGCCACGCCTGTCAGCTTG |
| | | ATAACATGACATTTTCCTTCATCTCAGAAATGTTTGACCTC |
| | | CAGGTAAGATGCTTCTCTCTGACATAGCTTTCCAGAAGCCC |
| | | CTGCCCTGGGGTGGAGGTGGGGACTCCATTTTAGAT |
| CSF2_g7_ | 1812 | AAGCCCTACTCCTGGGGGCTGGGGGCAGCAGCAAAAAGGAG |
| del | | TGGTGGAGAGTTCTTGTACCACTGTGGGCACTTGGCCACTG |
| | | CTCACCGACGAACGACATAGACCCGCCTGGAGCTGTACAAG |
| | | CAGGGCCTGCGGGGCAGCCTCACCAAGCTCAAGGGCCCCTT |
| | | GACCATGATGGCCAGCCACTACAAGCAGCACTGCCC |
| CD3E_24_ | 1813 | AATTCTGAAAATTCCTTCAGTGACAGGTGatcctcatcact |
| del | | gcctatgtttttatcatcctcatcaccgcctatgtttttat |
| | | catTGTGTTGCCATAGTATGTCAATATTACTGTGGTTCCAG |
| | | AGATGGAGACTTTATATGCTGGGGAGAAAGAAGGGAAATTG |
| | | GCAGAAGAAACCAGGACAATTTTAGAAAAGGCAAAT |
| CD3E_34_ | 1814 | GCCCTTTTGAATGGTCCTCCCTAAAGAGCCGGTGGTACCTG |
| del | | TTCTGGAGACCTGGATTACCTCTTGCCCTCAGGTAGAGATA |
| | | AAAGTTCGCATCTTCTGGTAATAACCACTTTGCTCCAATTC |
| | | TGAAAATTCCTTCAGTGACAGGTGatcctcatcactgccta |
| | | tgtttttatcatcctcatcaccgcctatgtttttat |
| SDN0017_C | 1815 | AGAGGAGTTTAACCATTAGGTAACATGACTTCGGCATCCCA |
| D40LG_40_ | | GCCTTTCCCCTTGGGTGGCTACCGCTCAGATGCTGTGTGAC |
| delstop | | TTACCAGATGTTGTTttaATGTGCCGCAATTTGAGGATTCT |
| | | GATCACCTGAAATGGAACCAAAAACTGTCAGGCTAAAATAA |
| | | TGCAAAAACTGCCCACAAAACTATCTGGTCCAGTTC |
| SDN0018_C | 1816 | AAATGGGAAACAGCTGACCGTTAAAAGACAAGGACTCTATT |
| D40LG_53_ | | ATATCTATGCCCAAGTCACCTTCTGTTCCAATCGGGAAGCT |
| delstop | | TCGAGTCAAGCTCCATTaCCCCCGGTAGATTCGAGAGAATC |
| | | TTACTCAGAGCTGCAAATACCCACAGTTCCGCCAAACCTTG |
| | | CGGGCAACAATCCATTCACTTGGGAGGAGTATTTGA |
| SDN0019_C | 1817 | TGCGAGGTACCTGAAGCGGCTGCAGCCGGGGACACTGCGGG |
| IITA_65_ | | CGCGGCAGCTGCTGGAGCTGCTGCACTGCGCCCACGAGGCC |
| delstop | | GAGGAGGCTGGAATTTGaCCCCGGCCGCCTCTCTTTTCTGG |
| | | GCACCCGCCTCACGCCTCCTGATGCACATGTACTGGGCAAG |
| | | GCCTTGGAGGCGGCGGGCCAAGACTTCTCCCTGGAC |
| SDN0020_C | 1818 | tctaaaaaaacaaaTTTAAATTAATTTTGAAAAAGTCAGCC |
| IITA_80_ | | GGACTTTGGGGGCCCGATTCAGCAGGAAGGGCAGGCCCAGC |
| delstop | | TCACTCACTTGAGGGtaaGGTGGCTGAGAGCTGCGAGACAC |
| | | CCTCGTCCCCGATCTTGTTCTCACTCAGCGCATCCAGGCTG |
| | | CAGGTGGAATCAGATGGGGGCCATCAGCTAGCGTCC |
| SDN0021_T | 1819 | TGCTGACCCCACTGTGCACCTCCTTCCCATTCACCCACCAG |
| 12_g1_TRB | | CTCAGCTCCACGTGGTCGGGGTAGAAGCCTGTGGCCAGGCA |
| C2_del | | CACCAGTGTGGCCTTTTGTGATGGCTCAAACACAGCGACCT |
| | | TGGGTGGGAACACGTTTTTCAGGTCCTCTGGAAAGGGAAGA |
| | | GGTAATGGGGCTAGGGTTGCTCTAAGAGCTGTCTGG |

### Example 2

Conditions were the same as Example 1, and in addition after transfection the cells were treated with the HDR enhancer M3814 for 24 hours to block the NHEJ pathway and 5 thereby increase the incorporation of the ssODN at the on-target side. The enhancer increased perfect HDR by 1.5 fold.

### Example 3: Culture of Jurkat Human T-Cell Leukemia Cell Line and Primary Human T-Cells

Human Jurkat T-cell leukemia cells (Leibniz Institute DSMZ-German Collection of Microorganisms and Cell Cultures GmbH (ACC 282)) were propagated in RPMI 1640 medium (Thermo Fisher Scientific) with 10% heat-inactivated fetal bovine serum (FBS) (ThermoFisher Scientific) supplemented with 1% penicillin-streptomycin antibiotic mix (ThermoFisher Scientific). Cells were cultured at 37° C., in 5% CO2 incubators and maintained at a density of 0.5 to 1.5×10⁶ cells mL⁻¹. 24 hours before transfection, cells were passaged at 0. 1×10⁶ cell mL⁻¹. Cell culture media supernatant was periodically tested for mycoplasma contamination using the MycoAlert PLUS mycoplasma detection kit (Lonza).

### Example 4: Primary T-Cell Isolation and Culture

T-cells were isolated from human peripheral blood obtained from healthy adults by immune-magnetic negative selection using the EasySep Human T-cell Isolation Kit (STEMCELL Technologies). After isolation, T-cells were activated in 25 μL mL⁻¹ ImmunoCult Human CD3/CD28/CD2 T-Cell Activator (STEMCELL Technologies) in ImmunoCult-XF T-Cell Expansion Medium (STEMCELL Technologies) containing 12.5 ng mL⁻¹ Human Recombinant IL-2, 5 ng mL⁻¹ IL-7, and 5 ng mL⁻¹ IL-15 (STEMCELL Technologies) and seeded at 1.0×10⁶ cells mL⁻¹. Until transfection 48 hours later, the cells were cultured at 37° C., in 5% CO2 incubators.

### Example 5: RNP Formulation

Ribonucleoprotein complexes (RNPs) were generated by incubating respective guide nucleic acids (gNAs) with MAD7 in the molar ratio of 3:2 gNA: MAD7 for 15 minutes at room temperature immediately before transfection. For Jurkat experiments, the RNP complexes were generated by mixing the respective gNA (150 μmol), MAD7 (100 μmol), and nuclease-free water, unless otherwise stated. For T-cell experiments, 1.6 μL of an aqueous solution of 15-50 kDa poly-L-glutamic acid (PGA, 100 μg μL⁻¹, Alamanda Polymers) was added to gNAs, followed by the addition of MAD7 and nuclease-free water.

### Example 6: Generation of Donor Template Via PCR Amplification

Donor templates comprising site-specific homology arms, respective promoter, and respective gene (GFP or Hu19 scFv-CD8a-CD28-CD3ζ CAR) were amplified from corresponding pTwist Ampicillin high-copy plasmids (Twist Bioscience) using homology arms-specific PCR primers. Donor templates were amplified in a two-step PCR program: initial denaturation at 98° C., for 30 seconds, cycle denaturation at 98° C., for 10 seconds, extension at 72° C., for 30 seconds per kb amplicon for 40-cycles with a hold at 72° C., for 10 minutes. Each 50 μL PCR reaction contained 10 ng amplification template (plasmid DNA), 0.5 μM homology arm-specific forward and reverse primers, nuclease-free water (IDT), 3% DMSO, and 1x Phusion High-Fidelity PCR Master Mix with HF Buffer (ThermoFisher Scientific). PCR products were purified using NucleoSpin Gel and PCR Clean-up Kit (Macherey-Nagel) with two 20 μL elutions. Purified HDR templates were collected and quantified on NanoDrop One Microvolume UV-Vis Spectrophotometer (ThermoFisher Scientific). Templates were concentrated using Amicon Ultra 0.5 mL 30K Centrifugal Filters: 100 μg DNA per unit was transferred, filled with nuclease-free water to 500 μL, and centrifuged at 10,000 g for 10 minutes to reduce volume to 50 μL. DNA was washed twice with nuclease-free water and recovered into a fresh tube by inversion and centrifugation at 10,000 g for 15 seconds. HDR templates were collected, diluted, and concentrations quantified using Qubit dsDNA HS Assay Kit (ThermoFisher Scientific). HDR templates of 0.5 to 1 μg μL⁻¹ were used for cellular studies.

### Example 7: Jurkat Cell Transfection

Lonza 4D Nucleofector with Shuttle unit (V4SC-2960 Nucleocuvette Strips) was used for transfection, following the manufacturer's instructions. For transfection, cells were harvested by centrifugation (200 g. RT, 5 minutes) and re-suspended in 20 μL at 10×10⁶ cells mL⁻¹ in the SF Cell Line Nucleofector X Kit buffer (Lonza), unless stated otherwise. The cell suspension was mixed with the RNPs, immediately transferred to the nucleocuvette, and transfected. After transfection, the cells were immediately re-suspended in the pre-warmed cultivation medium and plated onto 96-well, flat-bottom, non-cell culture treated plates (Falcon), and cultured at 37° C., in 5% CO₂ incubators and maintained at a density of 0.5 to 1.0×10⁶ cells mL⁻¹. After 48 hours, the cells were harvested for the viability assay and genomic DNA, as described below. For the Homology-Directed Repair Template insertion, the HDR template was added to the cells and the suspension transferred to the RNPs immediately before transfection. The transfection parameters, cell recovery step, and proliferation conditions as described in Example 1. The cells were harvested 48 hours post-transfection for the viability assessment, after 7 days for CAR insertion efficiency, or after 7 days, 14 days, and 21 days for GFP insertion efficiency.

### Example 8: Primary T-Cell Transfection

48 hours after isolation, the cells were harvested by centrifugation (300 g, RT, 5 minutes) and re-suspended in 20 μL at 50×10⁶ cells mL⁻¹ in the supplemented P3 Primary Cell Nucleofector Kit buffer (Lonza). The cells were mixed with HDR templates and the suspension transferred to the RNPs immediately before transfection (Nucleofection program EH-115). After transfection, 80 μL of pre-warmed cultivation medium without IL-2 was added to the electroporation cuvettes. When using M3814 (Selleckchem), 80 μL of pre-warmed cultivation medium containing 2 μM M3814 final concentration without IL-2 was added to the electroporation cuvettes. After 10 minutes of incubation at 37° C., T-cells were transferred onto 96-well, flat-bottom, non-cell culture treated plates (Falcon) containing pre-warmed cultivation medium pretreated with 2 μM M3814 final concentration and 12.5 ng mL⁻¹ IL-2. The cells were seeded at a density of 0.25×10⁶ cells mL⁻¹, or 1.3×10⁶ cells mL⁻¹ in the experiment with M3814, and kept at 37° C., in 5% CO₂ incubators. The viability assay was carried out 24 hours post-transfection after which the cells were reseeded in the fresh cultivation medium containing IL-2. Insertion efficiency of CAR was measured after 7 days, and 11 days or 13 days post-transfection.

### Example 9: Flow Cytometry

Flow cytometric assessments were carried out on a CytoFLEX S instrument (Beckmen Coulter) using a 96-well plate format. Measurements of cell viability, PDCD1 expression, GFP expression, and CAR expression were performed on 10,000 or 20,000 single cell events in Jurkat or primary T-cells, respectively.

For the cell viability and GFP knock-in measurements, approximately 250,000 cells per sample were transferred onto 96-well V-bottom cell culture plates and assessed following a series of consecutive washing and staining steps. The first step included centrifuging the cells at 300 g for 5 minutes at room temperature, discarding the supernatant, and washing cells in 150 μL Dulbecco's PBS/2% FBS (STEMCELL Technologies) or Cell Staining Buffer (Biolegend), respectively, followed by the second centrifugation and removal of supernatant. The final step included viability staining of cells using 150 μL Dulbecco's PBS/2% FBS with 7-amino-actinomycin D (7-AAD, 1:1,000: ThermoFisher) or 50 μL Cell Staining Buffer with Zombie Violet Dye (1:200: Biolegend), respectively. The measurements of cell viability and GFP expression were collected simultaneously for 7-AAD (excitation: yellow-green laser; emission; 561 nm), Zombie Violet (excitation: violet laser; emission 405 nm), and GFP (excitation: blue laser; emission 488 nm) as needed.

For detection of CAR knock-in efficiency, approx. 250,000 cells per sample were transferred onto 96-well V-bottom, washed as described above using Cell Staining Buffer, and re-suspended in 50 μL Cell Staining Buffer with PE Anti-Myc tag antibody [9E10] (1:50; Abcam) and Zombie Violet Dye (1:200; Biolegend) for 30 minutes. Afterwards, the cells were washed in two subsequent washing steps using 150 μL Cell Staining Buffer, and finally re-suspended in 100 μL Cell Staining Buffer for the flow cytometry measurements (excitation: yellow-green laser; emission: 561 nm).

For detection of PDCD1 knock-out efficiency, approx. 250,000 Jurkat cells per sample were transferred onto 96-well V-bottom cell culture plates and assessed following a series of consecutive washing and staining steps. The first step included centrifuging the cells at 300 g for 5 minutes at 4° C., and discarding the supernatant. Afterwards, the cells were stained using 100 μL Cell Staining Buffer (Biolegend) with APC/Cyanine7 anti-human CD279 (PD-1) antibody (1:100: Biolegend) and incubated for 30 minutes at 4° C., in the dark. The cells were then centrifuged at 300 g for 5 minutes at 4° C., and the supernatant discarded. The next step included two repeats of centrifugation at 300 g for 5 minutes at 4° C., supernatant removal, and cell washing in 150 μL ice-cold Cell Staining Buffer (Biolegend). In the final step, the cells were re-suspended in 100 μL Cell Staining Buffer for the flow cytometry measurements (excitation: red laser; emission: 633 nm).

### Example 10: DNA Extraction

Cells were harvested 48-h post-transfection by centrifugation (1,000 g, 10 minutes) in 96-well, V-bottom plates (Greiner), washed with PBS (Sigma Aldrich) and lysed in 20 μL Quick Extract DNA Extraction Solution (Epicentre, Lucigen). DNA was extracted following the manufacturer's protocol: 15 minutes at 65° C., 15 minutes at 68° C., 10 minutes at 95° C., cooled to 4° C., and stored at 4° C. Genomic DNA was diluted 20-fold in nuclease-free water before amplicon PCR reactions.

### Example 11: Amplicon Sequencing

Extracted genomic DNA was quantified using the NanoDrop (ThermoFisher Scientific). Amplicons were constructed in two PCR steps: in the first PCR, regions of interest (150-400 bp) were amplified from 10 to 30 ng of genomic DNA with primers containing Illumina forward and reverse adapters on both ends comprising loci-specific complementary sequences as shown in Table 12, using Phusion High-Fidelity PCR Master Mix (ThermoFisher Scientific). Amplification products were purified with Agencourt AMPure XP beads (Ramcon), using the sample to beads ratio of 1:1.8. The DNA was eluted from the beads with nuclease-free water and the size of the purified amplicons analyzed on a 2% agarose E-gel using the E-gel electrophoresis system (ThermoFisher Scientific). In the second PCR, unique pairs of Illumina-compatible indexes (Nextera XT Index Kit v2) were added to the amplicons using the KAPA HiFi HotStart Ready Mix (Roche). The amplified products were purified with Agencourt AMPure XP beads (Ramcon), using the sample to bead ratio of 1:1.8. The DNA was eluted from the beads with 10 mM Tris-HCl pH 8.5, 0.1% Tween 20. Sizes of the purified DNA fragments were validated on a 2% agarose gel using the E-gel electrophoresis system (ThermoFisher Scientific), quantified using Qubit dsDNA HS Assay Kit (Thermo Fisher) and then pooled in equimolar concentrations. Quality of the amplicon library was validated using Bioanalyzer, High Sensitivity DNA Kit (Agilent) before sequencing. The final library was sequenced on Illumina MiSeq System using the MiSeq Reagent Kit v.2 (300 cycles, 2×250 bp, paired-end reads). De-multiplexed FASTQ files were obtained from BaseSpace (Illumina).

TABLE 12
Primer sequences

| | SEQ | | SEQ | |
|---|---|---|---|---|
| | ID | | ID | |
| Name | NO | Forward primer | NO | Reverse primer |
| crCD247 | 385 | TGGGGAGGTAGCTGC | 684 | CTAGAAGTTCCCTGCCG |
| _1 | | AGAAT | | TCG |
| crCD247 | 386 | TGGGGAGGTAGCTGC | 685 | CTAGAAGTTCCCTGCCG |
| _2 | | AGAAT | | TCG |
| crCD247 | 387 | TGGGGATGTGTTCTC | 686 | GCCCCTCTGAACATCCA |
| _3 | | GTCAC | | TCA |
| crCD247 | 388 | GGTAGCACAGGGAGG | 687 | GCCCTTCCTCCAACTTT |
| _4 | | AGAGA | | CCA |
| crCD247 | 389 | TTAGTTGCCAAGGAG | 688 | GGCGAGGCTGACTTACG |
| _5 | | CGGAG | | TTA |
| crCD247 | 390 | GCCCTTCCTCCAACT | 689 | GGTAGCACAGGGAGGAG |
| _6 | | TTCCA | | AGA |
| crCD247 | 391 | GGTAGCACAGGGAGG | 690 | GCCCTTCCTCCAACTTT |
| _7 | | AGAGA | | CCA |
| crCD247 | 392 | CGTGTCTGGAGGACC | 691 | CTGGTTGTGGGCAGAGA |
| _8 | | AAGAG | | AGT |
| crCD247 | 393 | CTGGTTGTGGGCAGA | 692 | CGTGTCTGGAGGACCAA |
| _9 | | GAAGT | | GAG |
| crCD247 | 394 | TGCAGCTGGGATGAG | 693 | TGGAGCCTTGATTGTGG |
| _10 | | AAGTG | | GAG |
| crCD247 | 395 | TGCAGCTGGGATGAG | 694 | TGGAGCCTTGATTGTGG |
| _11 | | AAGTG | | GAG |
| crCD247 | 396 | GGCCTCACCTTACTC | 695 | ATCTTGCCCCTTGTCAG |
| _12 | | TGCAG | | GTG |
| crCD247 | 397 | GGCCTCACCTTACTC | 696 | ATCTTGCCCCTTGTCAG |
| _13 | | TGCAG | | GTG |
| crCD247 | 398 | GGCCTCACCTTACTC | 697 | ATCTTGCCCCTTGTCAG |
| _14 | | TGCAG | | GTG |
| crCD247 | 399 | TAAACCCAAGACTCT | 698 | TTAGTTGCCAAGGAGCG |
| _15 | | GGCGG | | GAG |
| crCD247 | 400 | ACAGCACCCATCTAC | 699 | GTCTGGCCTTTGAGTGG |
| _16 | | CAACG | | TGA |
| crCD247 | 401 | ACAGCACCCATCTAC | 700 | GTCTGGCCTTTGAGTGG |
| _17 | | CAACG | | TGA |
| crCD247 | 402 | ACAGCACCCATCTAC | 701 | GTCTGGCCTTTGAGTGG |
| _18 | | CAACG | | TGA |
| crCD247 | 403 | CAGGGGGATTATTCC | 702 | ATAATCTGGGCGTCTGC |
| _19 | | TGGGC | | AGG |
| crCD247 | 404 | TATGGCGCCCTTTGA | 703 | TGTGTTGCAGTTCAGCA |
| _20 | | GACAG | | GGA |
| crCD247 | 405 | GCCCCTGCCCCTCTT | 704 | TGGTTGCAGAGTGAGCT |
| _21 | | TTTAT | | GAG |
| crCD247 | 406 | GCCCCTGCCCCTCTT | 705 | TGGTTGCAGAGTGAGCT |
| _22 | | TTTAT | | GAG |
| crCD247 | 407 | TGGTTGCAGAGTGAG | 706 | GCCCCTGCCCCTCTTTT |
| _23 | | CTGAG | | TAT |
| crCD247 | 408 | TGGTTGCAGAGTGAG | 707 | GCCCCTGCCCCTCTTTT |
| _24 | | CTGAG | | TAT |
| crCD247 | 409 | GCCCCTGCCCCTCTT | 708 | TGGTTGCAGAGTGAGCT |
| _25 | | TTTAT | | GAG |
| crCD247 | 410 | GGTAGCACAGGGAGG | 709 | GCCCTTCCTCCAACTTT |
| _26 | | AGAGA | | CCA |
| crCTLA4 | 411 | ATCATGTAGGTTGCC | 710 | GGCCATGAAGGAGCATG |
| _1 | | GCACA | | AGT |
| crCTLA4 | 412 | TCACTGCCTTTGACT | 711 | TGAAGACCTGAACACCG |
| _2 | | GCTGA | | CTC |
| crCTLA4 | 413 | AAATCTGGGTTCCGT | 712 | AGGTGACTGAAGTCTGT |
| _3 | | TGCCT | | GCG |
| crCTLA4 | 414 | GGCCATGAAGGAGCA | 713 | ATCATGTAGGTTGCCGC |
| _4 | | TGAGT | | ACA |
| crCTLA4 | 415 | AGTCCTTGATTCTGT | 714 | CCTCCTCCATCTTCATG |
| _5 | | GTGGGT | | CTCC |
| crCTLA4 | 416 | CCTCCTCCATCTTCA | 715 | AGTCCTTGATTCTGTGT |
| _6 | | TGCTCC | | GGGT |
| crCTLA4 | 417 | AAGCTAGAAGGCAGA | 716 | ATCATGTAGGTTGCCGC |
| _7 | | AGGGC | | ACA |
| crCTLA4 | 418 | AAGCTAGAAGGCAGA | 717 | ATCATGTAGGTTGCCGC |
| _8 | | AGGGC | | ACA |
| crCTLA4 | 419 | GGCCATGAAGGAGCA | 718 | ATCATGTAGGTTGCCGC |
| _9 | | TGAGT | | ACA |
| crCTLA4 | 420 | GGCCATGAAGGAGCA | 719 | ATCATGTAGGTTGCCGC |
| _10 | | TGAGT | | ACA |
| crCTLA4 | 421 | CATGCTAGCAATGCA | 720 | TGATTTCCACTGGAGGT |
| _11 | | CGTGG | | GCC |
| crCTLA4 | 422 | CATGCTAGCAATGCA | 721 | TGATTTCCACTGGAGGT |
| _12 | | CGTGG | | GCC |
| crCTLA4 | 423 | CATCGCCAGCTTTGT | 722 | GAGCTCCACCTTGCAGA |
| _13 | | GTGTG | | TGT |
| crCTLA4 | 424 | AGTCCTTGATTCTGT | 723 | CCTCCTCCATCTTCATG |
| _14 | | GTGGGT | | CTCC |
| crCTLA4 | 425 | AGGTGACTGAAGTCT | 724 | AAATCTGGGTTCCGTTG |
| _15 | | GTGCG | | CCT |
| crCTLA4 | 426 | AGGTGACTGAAGTCT | 725 | AAATCTGGGTTCCGTTG |
| _16 | | GTGCG | | CCT |
| crCTLA4 | 427 | AGGTGACTGAAGTCT | 726 | AAATCTGGGTTCCGTTG |
| _17 | | GTGCG | | CCT |
| crCTLA4 | 428 | CATCTGCAAGGTGGA | 727 | GGTTGCCACCCACAATA |
| _18 | | GCTCA | | AGC |
| crCTLA4 | 429 | TCTGCAAGGTGGAGC | 728 | GGTTGCCACCCACAATA |
| _19 | | TCATG | | AGC |
| crCTLA4 | 430 | GCAATTTAGGGGTGG | 729 | CATCAGCACCACACTCA |
| _20 | | ACCTCA | | CCA |
| crCTLA4 | 431 | GCAATTTAGGGGTGG | 730 | CATCAGCACCACACTCA |
| _21 | | ACCTCA | | CCA |
| crCTLA4 | 432 | AATGTTGGGGAGTAG | 731 | ATCCCCATCAGACATGG |
| _22 | | AGCCC | | TGC |
| crCTLA4 | 433 | CAATGTTGGGGAGTA | 732 | GCACCACACTCACCATT |
| _23 | | GAGCCCT | | TTGCT |
| crCTLA4 | 434 | ATGTTGGGGAGTAGA | 733 | ATCCCCATCAGACATGG |
| _24 | | GCCCT | | TGC |
| crCTLA4 | 435 | AGTCCTTGATTCTGT | 734 | CCTCCTCCATCTTCATG |
| _25 | | GTGGGT | | CTCC |
| crCTLA4 | 436 | ATGTTGGGGAGTAGA | 735 | ATCCCCATCAGACATGG |
| _26 | | GCCCT | | TGC |
| crCTLA4 | 437 | ATGTTGGGGAGTAGA | 736 | ATCCCCATCAGACATGG |
| _27 | | GCCCT | | TGC |
| crCTLA4 | 438 | ATGTTGGGGAGTAGA | 737 | ATCCCCATCAGACATGG |
| _28 | | GCCCT | | TGC |
| crCTLA4 | 439 | ATGTTGGGGAGTAGA | 738 | ATCCCCATCAGACATGG |
| _29 | | GCCCT | | TGC |
| crCTLA4 | 440 | AGGGACCCAATATGT | 739 | TGCCTCAGCTCTTGGAA |
| _30 | | GTTGAGT | | ATTG |
| crCTLA4 | 441 | AGGGACCCAATATGT | 740 | TGCCTCAGCTCTTGGAA |
| _31 | | GTTGAGT | | ATTG |
| crCTLA4 | 442 | AGGGACCCAATATGT | 741 | TGCCTCAGCTCTTGGAA |
| _32 | | GTTGAGT | | ATTG |
| crCTLA4 | 443 | TGGTTAGAAGTGGCT | 742 | AGAATTGCCTCAGCTCT |
| _33 | | TCCGT | | TGGA |
| crCTLA4 | 444 | TGGTTAGAAGTGGCT | 743 | AGAATTGCCTCAGCTCT |
| _34 | | TTCCG | | TGGA |
| crCTLA4 | 445 | TGGTTAGAAGTGGCT | 744 | AGAATTGCCTCAGCTCT |
| _35 | | TTCCG | | TGGA |
| crCTLA4 | 446 | CCCTCTTACAACAGG | 745 | TGGGTTCCGCATCCAAC |
| _36 | | GGTCT | | TTT |
| crCTLA4 | 447 | CCCTCTTACAACAGG | 746 | TGGGTTCCGCATCCAAC |
| _37 | | GGTCT | | TTT |
| crCTLA4 | 448 | TGAAGACCTGAACAC | 747 | TCACTGCCTTTGACTGC |
| _38 | | CGCTC | | TGA |
| crCTLA4 | 449 | TGAAGACCTGAACAC | 748 | TCACTGCCTTTGACTGC |
| _39 | | CGCTC | | TGA |
| crCTLA4 | 450 | AAGCTAGAAGGCAGA | 749 | ATCATGTAGGTTGCCGC |
| _40 | | AGGGC | | ACA |
| crCTLA4 | 451 | AAGCTAGAAGGCAGA | 750 | ATCATGTAGGTTGCCGC |
| _41 | | AGGGC | | ACA |
| crLAG3_ | 452 | TAGTGAAGCCTCTCC | 751 | AGGGAGTGACACCTCAG |
| 1 | | AGCCA | | GG |
| crLAG3_ | 453 | CCAAGTGAGTGCAGG | 752 | GTGTCCAGAGAGCTCCA |
| 2 | | GTGAT | | CAC |
| crLAG3_ | 454 | TGGGGAAGCTGCTTT | 753 | TTTGGGTCCTGGCATTC |
| 3 | | GTGAG | | TGG |
| crLAG3_ | 455 | CTGGATCCCTGGGGA | 754 | TGGCGTTTGGGTCCTGG |
| 4 | | AGCTGCT | | CATTC |
| crLAG3_ | 456 | CCAAGTGAGTGCAGG | 755 | CCAGCCAAGGTCCTGAG |
| 5 | | GTGAT | | AAA |
| crLAG3_ | 457 | CCTTTTGGAGGGCTC | 756 | CCAGAGAGGCTTTCGGG |
| 6 | | AGCGCTG | | GTGGA |
| crLAG3_ | 458 | CTGAGATGGGGAGAG | 757 | TTCCGGAACCAATGCAC |
| 7 | | GGTGA | | AGA |
| crLAG3_ | 459 | TCCAGTGGGCTGATG | 758 | CTTGGGGCAGGAAGAGG |
| 8 | | AAGTC | | AAG |
| crLAG3_ | 460 | TCCAGTGGGCTGATG | 759 | CTTGGGGCAGGAAGAGG |
| 9 | | AAGTC | | AAG |
| crLAG3_ | 461 | GGATCTCTCAGAGCC | 760 | CTGTAGGTGAGGATGCA |
| 10 | | TCCGA | | GCC |
| crLAG3_ | 462 | GGATCTCTCAGAGCC | 761 | CTGTAGGTGAGGATGCA |
| 11 | | TCCGA | | GCC |
| crLAG3_ | 463 | GCCCAGCCTCTGTGC | 762 | GGGGGCAGGAAGGAGTT |
| 12 | | ATTGGTT | | GTGGT |
| crLAG3_ | 464 | GCCCAGCCTCTGTGC | 763 | GGGGGCAGGAAGGAGTT |
| 13 | | ATTGGTT | | GTGGT |
| crLAG3_ | 465 | GCCCAGCCTCTGTGC | 764 | GGGGGCAGGAAGGAGTT |
| 14 | | ATTGGTT | | GTGGT |
| crLAG3_ | 466 | CTTCCTCTTCCTGCC | 765 | ACCCACAGCAATGACGT |
| 15 | | CCAAG | | AGG |
| crLAG3_ | 467 | TGAGCCAGACCATCT | 766 | CAGTGAGGAAAGACCGG |
| 16 | | CCTGA | | GTC |
| crLAG3_ | 468 | CCTTTTGGAGGGCTC | 767 | CCAGAGAGGCTTTCGGG |
| 17 | | AGCGCTG | | GTGGA |
| crLAG3_ | 469 | TGAGCCAGACCATCT | 768 | CAGTGAGGAAAGACCGG |
| 18 | | CCTGA | | GTC |
| crLAG3_ | 470 | TGAGCCAGACCATCT | 769 | CAGTGAGGAAAGACCGG |
| 19 | | CCTGA | | GTC |
| crLAG3_ | 471 | GTCTGGAGCCCCCAA | 770 | CTGGGCCTGGCTCACAT |
| 20 | | CTCCCTT | | CCTCT |
| crLAG3_ | 472 | GTCTGGAGCCCCCAA | 771 | CTGGGCCTGGCTCACAT |
| 21 | | CTCCCTT | | CCTCT |
| crLAG3_ | 473 | GACCCGGTCTTTCCT | 772 | GAGGGCAGCTACTCCTT |
| 22 | | CACTG | | TCC |
| crLAG3_ | 474 | GACCCGGTCTTTCCT | 773 | GAGGGCAGCTACTCCTT |
| 23 | | CACTG | | TCC |
| crLAG3_ | 475 | GACCCGGTCTTTCCT | 774 | GAGGGCAGCTACTCCTT |
| 24 | | CACTG | | TCC |
| crLAG3_ | 476 | TGGCGACTTTACCCT | 775 | CTCTGGAACTTGTGCCC |
| 25 | | TCGAC | | AGT |
| crLAG3_ | 477 | TGGCGACTTTACCCT | 776 | CTCTGGAACTTGTGCCC |
| 26 | | TCGAC | | AGT |
| crLAG3_ | 478 | CCAAGTGAGTGCAGG | 777 | GTGTCCAGAGAGCTCCA |
| 27 | | GTGAT | | CAC |
| crLAG3_ | 479 | CCTTTTGGAGGGCTC | 778 | CCAGAGAGGCTTTCGGG |
| 28 | | AGCGCTG | | GTGGA |
| crLAG3_ | 480 | CCAAGTGAGTGCAGG | 779 | GTGTCCAGAGAGCTCCA |
| 29 | | GTGAT | | CAC |
| crLAG3_ | 481 | CCAAGTGAGTGCAGG | 780 | GTGTCCAGAGAGCTCCA |
| 30 | | GTGAT | | CAC |
| crLAG3_ | 482 | CCAAGTGAGTGCAGG | 781 | GTGTCCAGAGAGCTCCA |
| 31 | | GTGAT | | CAC |
| crLAG3_ | 483 | CCAAGTGAGTGCAGG | 782 | CCAGCCAAGGTCCTGAG |
| 32 | | GTGAT | | AAA |
| crLAG3_ | 484 | TCCTTTGGGTCACCT | 783 | CTGCTCCAAGAAGCCTC |
| 33 | | GGATC | | TCC |
| crLAG3_ | 485 | TCCTTTGGGTCACCT | 784 | CTGCTCCAAGAAGCCTC |
| 34 | | GGATC | | TCC |
| crLAG3_ | 486 | AGAACGCTTTGTGTG | 785 | TTTGGGTCCTGGCATTC |
| 35 | | GAGCT | | TGG |
| crLAG3_ | 487 | TTCCTGCACCCTGTT | 786 | GCAGAAGGCTGAGATCC |
| 36 | | TCTCC | | TGG |
| crLAG3_ | 488 | AGAACGCTTTGTGTG | 787 | TTTGGGTCCTGGCATTC |
| 37 | | GAGCT | | TGG |
| crLAG3_ | 489 | CTGGATCCCTGGGGA | 788 | TGGCGTTTGGGTCCTGG |
| 38 | | AGCTGCT | | CATTC |
| crLAG3_ | 490 | TTTCTCAGGACCTTG | 789 | AAGCCAGAGATCAGGTC |
| 39 | | GCTGG | | CCT |
| crLAG3_ | 491 | CTTTCCCAGCCTTGG | 790 | AAGCCAGAGATCAGGTC |
| 40 | | CAATG | | CCT |
| crLAG3_ | 492 | GCTGAATGACCCTGG | 791 | GGCTCCAGTCACCAAAA |
| 41 | | GACAA | | GGA |
| crLAG3_ | 493 | GCTGAATGACCCTGG | 792 | GGCTCCAGTCACCAAAA |
| 42 | | GACAA | | GGA |
| crLAG3_ | 494 | CCATAGGTGCCCAAC | 793 | TGAGGGCAAGTTCAGGG |
| 43 | | GCTCTGG | | TCCCA |
| crLAG3_ | 495 | CCATAGGTGCCCAAC | 794 | TGAGGGCAAGTTCAGGG |
| 44 | | GCTCTGG | | TCCCA |
| crLAG3_ | 496 | CCATAGGTGCCCAAC | 795 | TGAGGGCAAGTTCAGGG |
| 45 | | TGCTCGG | | TCCCA |
| crLAG3_ | 497 | GGCCTCTCTTTTGCT | 796 | GGTTGAGTGCTGGATTC |
| 46 | | CACCT | | GGA |
| crLAG3_ | 498 | CCATAGGTGCCCAAC | 797 | TGAGGGCAAGTTCAGGG |
| 47 | | GCTCTGG | | TCCCA |
| crLAG3_ | 499 | CCATAGGTGCCCAAC | 798 | TGAGGGCAAGTTCAGGG |
| 48 | | GCTCTGG | | TCCCA |
| crLAG3_ | 500 | CCATAGGTGCCCAAC | 799 | TGAGGGCAAGTTCAGGG |
| 49 | | GCTCTGG | | TCCCA |
| crLAG3_ | 501 | CCATAGGTGCCCAAC | 800 | TGAGGGCAAGTTCAGGG |
| 50 | | GCTCTGG | | TCCCA |
| crLAG3_ | 502 | CATCCTTCTCCTCCT | 801 | GACTGGGCTGCTGAGAT |
| 51 | | TCCGC | | CTG |
| crLAG3_ | 503 | CATCCTTCTCCTCCT | 802 | GACTGGGCTGCTGAGAT |
| 52 | | TCCGC | | CTG |
| crLAG3_ | 504 | CATCCTTCTCCTCCT | 803 | GACTGGGCTGCTGAGAT |
| 53 | | TCCGC | | CTG |
| crLAG3_ | 505 | GACGGTTGGTGGTCA | 804 | CACGCTCAGCACCGTGT |
| 54 | | AGAGA | | A |
| crLAG3_ | 506 | CGCTACACGGTGCTG | 805 | CACATACTCGAGGCCTG |
| 55 | | AGC | | GC |
| crLAG3_ | 507 | CTGAGATGGGGAGAG | 806 | TTCCGGAACCAATGCAC |
| 56 | | GGTGA | | AGA |
| crPDCD1 | 508 | TCTCTCAGACTCCCC | 807 | AGCTTGTCCGTCTGGTT |
| _1 | | AGACAGG | | GCT |
| crPDCD1 | 509 | CTAAGTCCCTGATGA | 808 | AGGAAGGAAGGCACAGT |
| _2 | | AGGCCCC | | GGATC |
| crPDCD1 | 510 | GCTGACTCCCTCTCC | 809 | CGCTAGGAAAGACAATG |
| _3 | | CTTTCTC | | GTGGC |
| crPDCD1 | 511 | TCTCTGTGGACTATG | 810 | CCAAGAGCAGTGTCCAT |
| _4 | | GGGAGCT | | CCTCA |
| crPDCD1 | 512 | CTGCAGCTTCTCCAA | 811 | GAGGTAGGTGCCGCTGT |
| _5 | | CACATCG | | CATT |
| crPDCD1 | 513 | GATGTGGAGGAAGAG | 812 | TACCTAAGAACCATCCT |
| _6 | | GGGGC | | GGCCG |
| crPDCD1 | 514 | CTGCAGCTTCTCCAA | 813 | GAGGTAGGTGCCGCTGT |
| _7 | | CACATCG | | CATT |
| crPDCD1 | 515 | CTGCAGCTTCTCCAA | 814 | GAGGTAGGTGCCGCTGT |
| _8 | | CACATCG | | CATT |
| crPDCD1 | 516 | CTGCAGCTTCTCCAA | 815 | GAGGTAGGTGCCGCTGT |
| _9 | | CACATCG | | CATT |
| crPDCD1 | 517 | CTGCAGCTTCTCCAA | 816 | GAGGTAGGTGCCGCTGT |
| _10 | | CACATCG | | CATT |
| crPDCD1 | 518 | GCGTGACTTCCACAT | 817 | AGCTCCTGATCCTGTGC |
| _11 | | GAGCG | | AG |
| crPDCD1 | 519 | GCGTGACTTCCACAT | 818 | AGCTCCTGATCCTGTGC |
| _12 | | GAGCG | | AG |
| crPDCD1 | 520 | GCGTGACTTCCACAT | 819 | AGCTCCTGATCCTGTGC |
| _13 | | GAGCG | | AG |
| crPDCD1 | 521 | CTCTAGTCTGCCCTC | 820 | GACCCAGACTAGCAGCA |
| _14 | | ACCCCT | | CCAG |
| crPDCD1 | 522 | CTCTAGTCTGCCCTC | 821 | GACCCAGACTAGCAGCA |
| _15 | | ACCCCT | | CCAG |
| crPDCD1 | 523 | GATGTGGAGGAAGAG | 822 | TACCTAAGAACCATCCT |
| _16 | | GGGGC | | GGCCG |
| crPDCD1 | 524 | CTCTAGTCTGCCCTC | 823 | GACCCAGACTAGCAGCA |
| _17 | | ACCCCT | | CCAG |
| crPDCD1 | 525 | CTCTAGTCTGCCCTC | 824 | GACCCAGACTAGCAGCA |
| _18 | | ACCCCT | | CCAG |
| crPDCD1 | 526 | CTCTAGTCTGCCCTC | 825 | GACCCAGACTAGCAGCA |
| _19 | | ACCCCT | | CCAG |
| crPDCD1 | 527 | CAGCTCAGGGTAAGC | 826 | GGTCTTCTCTCGCCACT |
| _20 | | AGCTCAT | | GGAAA |
| crPDCD1 | 528 | CAGCTCAGGGTAAGC | 827 | GGTCTTCTCTCGCCACT |
| _21 | | AGCTCAT | | GGAAA |
| crPDCD1 | 529 | GCTGACTCCCTCTCC | 828 | CGCTAGGAAAGACAATG |
| _22 | | CTTTCTC | | GTGGC |
| crPDCD1 | 530 | TCTCTGTGGACTATG | 829 | CCAAGAGCAGTGTCCAT |
| _23 | | GGGAGCT | | CCTCA |
| crPDCD1 | 531 | GATGTGGAGGAAGAG | 830 | TACCTAAGAACCATCCT |
| _24 | | GGGGC | | GGCCG |
| crPDCD1 | 532 | GCCACCATTGTCTTT | 831 | TTCTCCTGAGGAAATGC |
| _25 | | CCTAGCG | | GCTGA |
| crPDCD1 | 533 | GATGTGGAGGAAGAG | 832 | TACCTAAGAACCATCCT |
| _26 | | GGGGC | | GGCCG |
| crPDCD1 | 534 | TCTCTCAGACTCCCC | 833 | AGCTTGTCCGTCTGGTT |
| _27 | | AGACAGG | | GCT |
| crPDCD1 | 535 | TCTCTCAGACTCCCC | 834 | AGCTTGTCCGTCTGGTT |
| _28 | | AGACAGG | | GCT |
| crPDCD1 | 536 | TCTCTCAGACTCCCC | 835 | AGCTTGTCCGTCTGGTT |
| _29 | | AGACAGG | | GCT |
| crPDCD1 | 537 | TCTCTCAGACTCCCC | 836 | AGCTTGTCCGTCTGGTT |
| _30 | | AGACAGG | | GCT |
| crPTPN1 | 538 | TGGTGTCTGTCTTCT | 837 | TTCTTGTACGAGAGAGC |
| _1 | | GTCAGC | | CAGAG |
| crPTPN1 | 539 | CGAAATGCAGGCAGC | 838 | CACCCAAATATCACTGG |
| _2 | | AAGCTAT | | TGTGGA |
| crPTPN1 | 540 | CTCTGGGAAAGAAGC | 839 | GGTAACATCTTGCCAGA |
| _3 | | AGAGAA | | CCCA |
| CrPTPN1 | 541 | TTCTGTCTACCTCTG | 840 | GAAATACGACGTTGGTG |
| 1_4 | | TATGTTTGC | | GAGGAG |
| crPTPN1 | 542 | CTTGGACTAGGCTGG | 841 | TGGTCAGAAAACACTGT |
| 1_5 | | GGAGTA | | GAAAAG |
| crPTPN1 | 543 | AGGACGTCAGTTTCA | 842 | GATCAGCCCCTTAACAC |
| 1_6 | | AGTCTCTC | | GACTC |
| crPTPN1 | 544 | TCCAAGCATGGTTTT | 843 | GTTGTTGTGGAAAGTAG |
| 1_7 | | ACCACTTC | | TGCTGA |
| crPTPN1 | 545 | CGCACACAATTCTGA | 844 | AGGTACAGAGGTGCTAG |
| 1_8 | | ACATTTCC | | GAATC |
| crPTPN1 | 546 | CCCTTGGAGGAATGT | 845 | GAACAAAATCTCCAGGG |
| 1_9 | | GTCTACTTTT | | TGGCTC |
| crPTPN1 | 547 | G+F96AACAAAATCT | 846 | CCCTTGGAGGAATGTGT |
| 1_10 | | CCAGGGTGGCTC | | CTACTTTT |
| crPTPN6 | 548 | CTCTACTCCTGCACC | 847 | GCGGGTACTTGAGGTGG |
| _1 | | GACTGG | | ATGAT |
| crPTPN6 | 549 | GGGGGATCAGGTGAC | 848 | GGAGCCCTCACCTCTCA |
| _2 | | CCATA | | CTA |
| crPTPN6 | 550 | CCCGATGGATGCCCT | 849 | GAGGGTGGAGACCTGTG |
| _3 | | CTTTG | | AGA |
| crPTPN6 | 551 | GCACAGGCACCATCA | 850 | TGAACTTGTACTGCGCC |
| _4 | | TTGTC | | TCC |
| crPTPN6 | 552 | CGACCCTCCCTTTCC | 851 | AGAACAAGTCCAGGGAG |
| _5 | | AGAAC | | GGA |
| crPTPN6 | 553 | GATGGTGAGGTAAGG | 852 | TACCTGACGGAGAGCGA |
| _6 | | GCCTG | | GAA |
| crPTPN6 | 554 | GGCCCCTCTCTGTGA | 853 | ACTGAGCACAGAAAGCA |
| _7 | | ATGTC | | CGA |
| crPTPN6 | 555 | GTGGCCTGGGTCTTA | 854 | CTGCCTTACCTCGCACA |
| _8 | | CCTTC | | TGA |
| CrPTPN6 | 556 | GTGGCCTGGGTCTTA | 855 | CTGCCTTACCTCGCACA |
| _9 | | CCTTC | | TGA |
| crPTPN6 | 557 | GTGGCCTGGGTCTTA | 856 | CTGCCTTACCTCGCACA |
| _10 | | CCTTC | | TGA |
| crPTPN6 | 558 | GTGGCCTGGGTCTTA | 857 | CTGCCTTACCTCGCACA |
| _11 | | CCTTC | | TGA |
| crPTPN6 | 559 | GTGGCCTGGGTCTTA | 858 | CTGCCTTACCTCGCACA |
| _12 | | CCTTC | | TGA |
| crPTPN6 | 560 | GTGGCCTGGGTCTTA | 859 | CTGCCTTACCTCGCACA |
| _13 | | CCTTC | | TGA |
| crPTPN6 | 561 | CTGGACGTTTCTTGT | 860 | GGTCCCCAGCCTTGAAT |
| _14 | | GCGTG | | TCA |
| crPTPN6 | 562 | CTGGACGTTTCTTGT | 861 | GGTCCCCAGCCTTGAAT |
| _15 | | GCGTG | | TCA |
| crPTPN6 | 563 | GGAGGGTCTGCCTGG | 862 | GTAGACAAAGGCGCCTG |
| _16 | | GCTTGAA | | AGGCC |
| crPTPN6 | 564 | GATGGTGAGGTAAGG | 863 | TACCTGACGGAGAGCGA |
| _17 | | GCCTG | | GAA |
| crPTPN6 | 565 | CTGAGGCTCCTGTCT | 864 | GTAGACAAAGGCGCCTG |
| _18 | | GTGAC | | AGG |
| crPTPN6 | 566 | CTCAAGTCCTGTGAA | 865 | CAGAAGCTCACATCTGG |
| _19 | | TGGCCT | | GGG |
| crPTPN6 | 567 | CTCAAGTCCTGTGAA | 866 | CAGAAGCTCACATCTGG |
| _20 | | TGGCCT | | GGG |
| crPTPN6 | 568 | GACTTCTCGCTCTTC | 867 | GCAAGGAGGGGAAGGTG |
| _21 | | CCCAC | | TC |
| crPTPN6 | 569 | GACTTCTCGCTCTTC | 868 | GCAAGGAGGGGAAGGTG |
| _22 | | CCCAC | | TC |
| crPTPN6 | 570 | GACACCTTCCCCTCC | 869 | CGGTATCCTGGGTGAAT |
| _23 | | TTGC | | GGG |
| crPTPN6 | 571 | CCGATGGATGCCCTC | 870 | GAGGGTGGAGACCTGTG |
| _24 | | TTTGG | | AGA |
| crPTPN6 | 572 | GCTGATGCTCATTTC | 871 | GAGGGTGGAGACCTGTG |
| _25 | | CCCAC | | AGA |
| crPTPN6 | 573 | GATGCTCATTTCCCC | 872 | GAGGGTGGAGACCTGTG |
| _26 | | ACCCA | | AGA |
| crPTPN6 | 574 | CTCTCCGCCCACTCC | 873 | CAGCACAGGCCCTGAAC |
| _27 | | CAGTTGA | | CACTG |
| crPTPN6 | 575 | CTTGCATGGGTGAGG | 874 | ACCCGGCCTTTCTCCAC |
| _28 | | GTGGCAG | | CTCTC |
| crPTPN6 | 576 | GCTCACTGTCTTGGG | 875 | TGCCCTGGCATCTGACT |
| _29 | | GTGCGTC | | GCTCT |
| crPTPN6 | 577 | GCTCACTGTCTTGGG | 876 | TGCCCTGGCATCTGACT |
| _30 | | GTGCGTC | | GCTCT |
| crPTPN6 | 578 | GCTCACTGTCTTGGG | 877 | TGCCCTGGCATCTGACT |
| _31 | | GTGCGTC | | GCTCT |
| crPTPN6 | 579 | CCCATCCGTCCATCC | 878 | TTCGGTTGTGTCATGCT |
| _32 | | AACAA | | CCC |
| crPTPN6 | 580 | CCCATCCGTCCATCC | 879 | TTCGGTTGTGTCATGCT |
| _33 | | AACAA | | CCC |
| crPTPN6 | 581 | CGACCCTCCCTTTCC | 880 | AGAACAAGTCCAGGGAG |
| _34 | | AGAAC | | GGA |
| crPTPN6 | 582 | GGCCCTACTCTGTGA | 881 | GCCAGATCTCCCGAATC |
| _35 | | CCAAC | | AGG |
| crPTPN6 | 583 | CACGGTAGACAGGAG | 882 | GCACAAGAGAGTGGCCA |
| _36 | | GCAAG | | AAA |
| crPTPN6 | 584 | GTCGGGTAGGGTGAG | 883 | ATCATCCTCACCTGCAG |
| _37 | | ATGGA | | TGC |
| crPTPN6 | 585 | CCTGATTCGGGAGAT | 884 | AACAGCTCATGGCACTT |
| _38 | | CTGGC | | AGC |
| crPTPN6 | 586 | CCTGATTCGGGAGAT | 885 | AACAGCTCATGGCACTT |
| _39 | | CTGGC | | AGC |
| crPTPN6 | 587 | CCTGATTCGGGAGAT | 886 | AACAGCTCATGGCACTT |
| _40 | | CTGGC | | AGC |
| crPTPN6 | 588 | GCTTGACTGGCCTCT | 887 | TCAATGTCACAGTCCAG |
| _41 | | GATGG | | GCC |
| crPTPN6 | 589 | GGCCTGGACTGTGAC | 888 | AGAGGGACAGTGGGAAG |
| _42 | | ATTGA | | GTG |
| crPTPN6 | 590 | GGCCTGGACTGTGAC | 889 | AGAGGGACAGTGGGAAG |
| _43 | | ATTGA | | GTG |
| crPTPN6 | 591 | GGCCTGGACTGTGAC | 890 | AGAGGGACAGTGGGAAG |
| _44 | | ATTGA | | GTG |
| crPTPN6 | 592 | CTCTACTCCTGCAC | 891 | GCGGGTACTTGAGGTGG |
| _45 | | CGACTGG | | ATGAT |
| crPTPN6 | 593 | TTCAGGCTTGGTTCT | 892 | CAGGTCAGGAGACAGCA |
| _46 | | CACCC | | CAG |
| crPTPN6 | 594 | GCCTCTGTCCTCTAG | 893 | TGACCGCTGCTTCTTCA |
| _47 | | GAGCT | | CTT |
| crPTPN6 | 595 | GCCTCTGTCCTCTAG | 894 | TGACCGCTGCTTCTTCA |
| _48 | | GAGCT | | CTT |
| crPTPN6 | 596 | CTGTGCTGTCTCCTG | 895 | AAGAGCTGTACCATGGC |
| _49 | | ACCTG | | CAC |
| crPTPN6 | 597 | CTGTGCTGTCTCCTG | 896 | AAGAGCTGTACCATGGC |
| _50 | | ACCTG | | CAC |
| crPTPN6 | 598 | CTGTGCTGTCTCCTG | 897 | AAGAGCTGTACCATGGC |
| _51 | | ACCTG | | CAC |
| crPTPN6 | 599 | ATGGAGGGGAGAAGT | 898 | GGAGGGGATGGAGGGTA |
| _52 | | TTGCG | | GG |
| crPTPN6 | 600 | GGCCCCTCTCTGTGA | 899 | ACTGAGCACAGAAAGCA |
| _53 | | ATGTC | | CGA |
| crTIGIT | 601 | AAGAGGCCACATCTG | 900 | GTGGCATGCTCTTGGAG |
| _1 | | CTTCC | | TCT |
| CrTIGIT | 602 | GGCTCCAGTCCCATG | 901 | TTCTAGTCAACGCGACC |
| _2 | | GTTAC | | ACC |
| CrTIGIT | 603 | ATGTCACCTCTCCTC | 902 | TCTCCCAGTGTACGTCC |
| _3 | | CACCA | | CAT |
| crTIGIT | 604 | CCCAGGACTCACATG | 903 | GAAGGATGGGGAGATGT |
| _4 | | TGCTT | | GCC |
| CrTIGIT | 605 | ATGTCACCTCTCCTC | 904 | TCTCCCAGTGTACGTCC |
| _5 | | CACCA | | CAT |
| crTIGIT | 606 | AAGAGGCCACATCTG | 905 | GTGGCATGCTCTTGGAG |
| _6 | | CTTCC | | TCT |
| CrTIGIT | 607 | ATGTCACCTCTCCTC | 906 | TCTCCCAGTGTACGTCC |
| _7 | | CACCA | | CAT |
| CrTIGIT | 608 | ATGTCACCTCTCCT | 907 | TCTCCCAGTGTACGTCC |
| _8 | | CCACCA | | CAT |
| CrTIGIT | 609 | GGCACATCTCCCCAT | 908 | TGCTGTGCAGTGTTTCA |
| _9 | | CCTTC | | GGA |
| CrTIGIT | 610 | GGCACATCTCCCCAT | 909 | TGCTGTGCAGTGTTTCA |
| _10 | | CCTTC | | GGA |
| crTIGIT | 611 | GGCACATCTCCCCAT | 910 | TGCTGTGCAGTGTTTCA |
| _11 | | CCTTC | | GGA |
| CrTIGIT | 612 | GGCACATCTCCCCAT | 911 | TGCTGTGCAGTGTTTCA |
| _12 | | CCTTC | | GGA |
| CrTIGIT | 613 | GGTTACACAAAGGGC | 912 | GCCGGAGCCATTACCTT |
| _13 | | TTGGC | | TCT |
| CrTIGIT | 614 | GTCCTCCCTCTAGTG | 913 | TCTGGGTCTCTCTCTGG |
| _14 | | GCTGA | | GTG |
| crTIGIT | 615 | GTCCTCCCTCTAGTG | 914 | TCTGGGTCTCTCTCTGG |
| _15 | | GCTGA | | GTG |
| CrTIGIT | 616 | AGCTGTAACGCGGTT | 915 | CCATTCCTCCTGTCCAG |
| _16 | | GAGAA | | CTG |
| crTIGIT | 617 | AGCTGTAACGCGGTT | 916 | CCATTCCTCCTGTCCAG |
| _17 | | GAGAA | | CTG |
| crTIGIT | 618 | AGTTTGCTGGTGTGC | 917 | CATGCAGCTCGGCACAG |
| _18 | | ATGTGTGT | | TCCTC |
| CrTIGIT | 619 | AGTTTGCTGGTGTGC | 918 | CATGCAGCTCGGCACAG |
| _19 | | ATGTGTGT | | TCCTC |
| CrTIGIT | 620 | AGTTTGCTGGTGTGC | 919 | CATGCAGCTCGGCACAG |
| _20 | | ATGTGTGT | | TCCTC |
| crTIGIT | 621 | AGTTTGCTGGTGTGC | 920 | CATGCAGCTCGGCACAG |
| _21 | | ATGTGTGT | | TCCTC |
| crTIGIT | 622 | AGAAGAAAGCCCTCA | 921 | TGCAGTTACCCAGGCTT |
| _22 | | GAATCCA | | CTG |
| crTIGIT | 623 | TGTGGAAGGTGACCT | 922 | AGAAGATGCCTCTGGTT |
| _23 | | CAGGA | | GCT |
| crTIGIT | 624 | GGAGGAGCAACAGGA | 923 | TGGTGGAGGAGAGGTGA |
| _24 | | TGGAC | | CAT |
| CrTIGIT | 625 | GAAGCTGTGTCCAGG | 924 | CGCAGCACTGATGGAGA |
| _25 | | CAGAA | | GTA |
| crTIGIT | 626 | GAAGCTGTGTCCAGG | 925 | CGCAGCACTGATGGAGA |
| _26 | | CAGAA | | GTA |
| crTIGIT | 627 | GGAGGAGCAACAGGA | 926 | TGGTGGAGGAGAGGTGA |
| _27 | | TGGAC | | CAT |
| crTIGIT | 628 | CCCAGGACTCACATG | 927 | GAAGGATGGGGAGATGT |
| _28 | | TGCTT | | GCC |
| crTIGIT | 629 | CCCAGGACTCACATG | 928 | GAAGGATGGGGAGATGT |
| _29 | | TGCTT | | GCC |
| CrTIGIT | 630 | CCCAGGACTCACATG | 929 | GAAGGATGGGGAGATGT |
| _30 | | TGCTT | | GCC |
| crTIGIT | 631 | ATGTCACCTCTCCTC | 930 | TCTCCCAGTGTACGTCC |
| _31 | | CACCA | | CAT |
| crTIM3_ | 632 | GGCCATCCTTGTATC | 931 | GCGGCTACTGCTCATGT |
| 1 | | TCTCCC | | GAT |
| crTIM3_ | 633 | GCACGGAGATATCCA | 932 | GACATTAGCCAAGGTCA |
| 2 | | TGCCT | | Ccc |
| crTIM3_ | 634 | GGCCATCCTTGTATC | 933 | GCGGCTACTGCTCATGT |
| 3 | | TCTCCC | | GAT |
| crTIM3_ | 635 | TGTCTCCACCACTTC | 934 | ACATTAGCCAAGGTCAC |
| 4 | | CCTCT | | CCC |
| crTIM3_ | 636 | GATCCGGCAGCAGTA | 935 | ATGCCTATCTGCCCTGC |
| 5 | | GATCC | | TTC |
| CrTIM3_ | 637 | CCCTTGTCCTCTGTA | 936 | GCGGCTACTGCTCATGT |
| 6 | | CAGCA | | GAT |
| crTIM3_ | 638 | TCTCCTTTGCGGAAA | 937 | ATGCAGGGTCCTCAGAA |
| 7 | | TCCCC | | GTG |
| crTIM3_ | 639 | GATCCGGCAGCAGTA | 938 | ATGCCTATCTGCCCTGC |
| 8 | | GATCC | | TTC |
| crTIM3_ | 640 | GATCCGGCAGCAGTA | 939 | ATGCCTATCTGCCCTGC |
| 9 | | GATCC | | TTC |
| crTIM3_ | 641 | GATCCGGCAGCAGTA | 940 | ATGCCTATCTGCCCTGC |
| 10 | | GATCC | | TTC |
| crTIM3_ | 642 | GATCCGGCAGCAGTA | 941 | ATGCCTATCTGCCCTGC |
| 11 | | GATCC | | TTC |
| crTIM3_ | 643 | GATCCGGCAGCAGTA | 942 | ATGCCTATCTGCCCTGC |
| 12 | | GATCC | | TTC |
| crTIM3_ | 644 | GCAAATGTCCACTCA | 943 | GGAGCCTGTCCTGTGTT |
| 13 | | CCTGG | | TGA |
| crTIM3_ | 645 | GCAAATGTCCACTCA | 944 | GGAGCCTGTCCTGTGTT |
| 14 | | CCTGG | | TGA |
| crTIM3_ | 646 | TCTTAGTGGCCCTCC | 945 | CGCAAAGGAGATGTGTC |
| 15 | | TCCAG | | CCT |
| crTIM3_ | 647 | CCCTTGTCCTCTGTA | 946 | GCGGCTACTGCTCATGT |
| 16 | | CAGCA | | GAT |
| crTIM3_ | 648 | TCTTAGTGGCCCTCC | 947 | CGCAAAGGAGATGTGTC |
| 17 | | TCCAG | | CCT |
| crTIM3_ | 649 | TCTTAGTGGCCCTCC | 948 | CGCAAAGGAGATGTGTC |
| 18 | | TCCAG | | CCT |
| crTIM3_ | 650 | ACTGAGCATCACCAA | 949 | CAGTGGGATCTACTGCT |
| 19 | | TGGGG | | GCC |
| crTIM3_ | 651 | GTCCCCTGGTGGTAA | 950 | ACGTAGGTATCCAGGCA |
| 20 | | GCATC | | GGT |
| crTIM3_ | 652 | GTCCCCTGGTGGTAA | 951 | ACGTAGGTATCCAGGCA |
| 21 | | GCATC | | GGT |
| crTIM3_ | 653 | AAAGATTCCCTCCTC | 952 | AGGTTTGGAAGCTGAGG |
| 22 | | TGCCC | | GTG |
| crTIM3_ | 654 | GCCAGCTAAAGATTC | 953 | CTTGCTGCCCCTTTGAT |
| 23 | | CCTCCT | | TCC |
| crTIM3_ | 655 | GCACGGAGATATCCA | 954 | TGTTTCTGACATTAGCC |
| 24 | | TGCCT | | AAGGT |
| crTIM3_ | 656 | CCCTTGTCCTCTGTA | 955 | GCGGCTACTGCTCATGT |
| 25 | | CAGCA | | GAT |
| crTIM3_ | 657 | TGAGTACAACATAGC | 956 | CGGAGTAGAATTCATTT |
| 26 | | TCACAAA | | CAAATAGG |
| crTIM3_ | 658 | TGAGTACAACATAGC | 957 | CGGAGTAGAATTCATTT |
| 27 | | TCACAAA | | CAAATAGG |
| crTIM3_ | 659 | CAAGGACAAGGTGGG | 958 | TCCTCTCTCTCTCTCTC |
| 28 | | CATGAAG | | TCTCTCT |
| crTIM3_ | 660 | CACAGATCCCTGCTC | 959 | AGGACTCAGCCATCCTG |
| 29 | | CGATG | | TGA |
| crTIM3_ | 661 | CACAGATCCCTGCTC | 960 | AGGACTCAGCCATCCTG |
| 30 | | CGATG | | TGA |
| crTIM3_ | 662 | CGCCGAAGATAAGAG | 961 | CAGCCATCCTGTGATGT |
| 31 | | CCAGA | | TGT |
| crTIM3_ | 663 | GGATTTGGATGGACA | 962 | TGGCCAATGACTTACGG |
| 32 | | AAAGGGT | | GAC |
| crTIM3_ | 664 | GGATTTGGATGGACA | 963 | TGGCCAATGACTTACGG |
| 33 | | AAAGGGT | | GAC |
| crTIM3_ | 665 | CAAAGCCCCAGGACA | 964 | GCGTGCTTCCAGTGAAC |
| 34 | | GGATT | | CTA |
| crTIM3_ | 666 | CAAAGCCCCAGGACA | 965 | GCGTGCTTCCAGTGAAC |
| 35 | | GGATT | | CTA |
| crTIM3_ | 667 | CCCTTGTCCTCTGTA | 966 | GCGGCTACTGCTCATGT |
| 36 | | CAGCA | | GAT |
| crTIM3_ | 668 | CAAAGCCCCAGGACA | 967 | GCGTGCTTCCAGTGAAC |
| 37 | | GGATT | | CTA |
| crTIM3_ | 669 | CAAAGCCCCAGGACA | 968 | GCGTGCTTCCAGTGAAC |
| 38 | | GGATT | | CTA |
| crTIM3_ | 670 | CAAAGCCCCAGGACA | 969 | GCGTGCTTCCAGTGAAC |
| 39 | | GGATT | | CTA |
| crTIM3_ | 671 | CATTGGGCTCCTCCA | 970 | GCTGTCTCTTTGGGAAA |
| 40 | | CTTCA | | GCC |
| crTIM3_ | 672 | CATTGGGCTCCTCCA | 971 | GCTGTCTCTTTGGGAAA |
| 41 | | CTTCA | | GCC |
| crTIM3_ | 673 | CATTGGGCTCCTCCA | 972 | GCTGTCTCTTTGGGAAA |
| 42 | | CTTCA | | GCC |
| crTIM3_ | 674 | CATTGCAAAGCGACA | 973 | CCGTGTTACCTGGGAAA |
| 43 | | ACCCA | | TGC |
| crTIM3_ | 675 | CATTGCAAAGCGACA | 974 | CCGTGTTACCTGGGAAA |
| 44 | | ACCCA | | TGC |
| crTIM3_ | 676 | CATTGCAAAGCGACA | 975 | CCGTGTTACCTGGGAAA |
| 45 | | ACCCA | | TGC |
| crTIM3_ | 677 | CATTGCAAAGCGACA | 976 | CCGTGTTACCTGGGAAA |
| 46 | | ACCCA | | TGC |
| crTIM3_ | 678 | CAGTGCAGGTCCCAG | 977 | AGTGGAGGAGCCCAATG |
| 47 | | TTCAA | | AGT |
| crTIM3_ | 679 | CAGTGCAGGTCCCAG | 978 | AGTGGAGGAGCCCAATG |
| 48 | | TTCAA | | AGT |
| crTIM3_ | 680 | TCAAACACAGGACAG | 979 | AACAGGACTGCAGCAGT |
| 49 | | GCTCC | | AGC |
| crTIM3_ | 681 | TCTCCTTTGCGGAAA | 980 | ATGCAGGGTCCTCAGAA |
| 50 | | TCCCC | | GTG |
| crTIM3_ | 682 | TCTCCTTTGCGGAAA | 981 | ATGCAGGGTCCTCAGAA |
| 51 | | TCCCC | | GTG |
| crAAVS1 | 683 | CATCTCTCCTCCCTC | 982 | AAGAGGATGGAGAGGTG |
| | | ACCCA | | GCT |

### Example 12: NGS Data Analysis

Initial quality assessment of the obtained reads was performed with FastQC36. The sequencing data were aligned and analyzed with the CRISPResso2 software, using CRISPRessoBatch command with the parameters—cleavage_offset 1—quantification_window_size 10—quantification_window_center 1—expand_ambiguous_alignments for the INDEL frequency analysis. For the ORF disruption analysis, CRISPRessoBatch command with the parameters—cleavage_offset 1—coding_seq <EXON_SEQ>—quantification_window_size 0—quantification_window_center 1—expand_ambiguous_alignments was used. Modification rates from the CRISPResso2 software output were analyzed in Excel.

### Example 13: CRISPR-MAD7 Platform for Human Genome Editing Using the Jurkat T-Cell Leukemia Cell Line

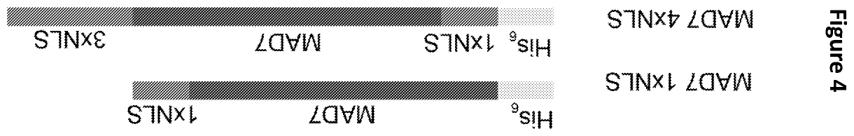

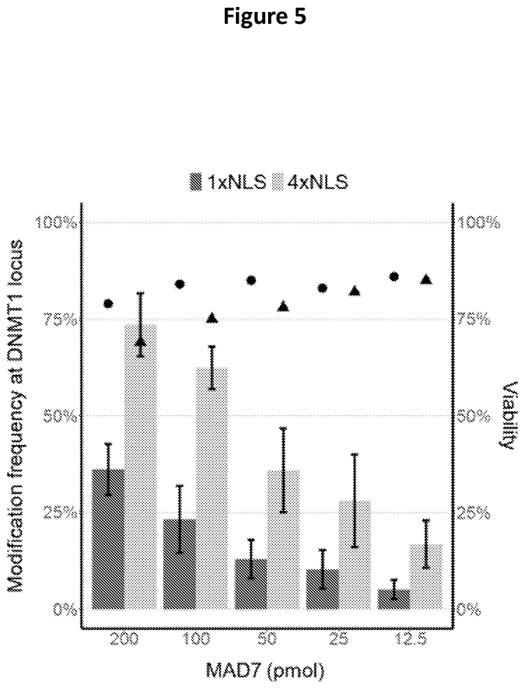

MAD7 nuclease comprising a His6 tag and either one (MAD7-INLS) or four (MAD7-4NLS) nuclear localization signals (NLS) were used (FIG. 5). RNPs were generated as described in Example 5. Editing frequency of the MAD7 nuclease complexed with one or more guide nucleic acids comprising a spacer sequence of SEQ ID NOs: 86-384 as shown in Table 5 was determined by nucleofection of RNPs in Jurkat T-cells using the Lonza recommended nucleofection program SE-CL-120 (Example 7), followed by genomic DNA extraction (Example 10), amplification of the edited locus and targeted next-generation sequencing (Example 11) for identification of the edits, and finally by computational analysis (Example 12) of modification frequency using the CRISPResso2 algorithm.

Firstly, using a gNA targeting the DNMT1 locus, the editing frequency of MAD7 comprising either one or four NLS complexed with the respective gNA was compared. RNP concentration-dependent modification efficiency was observed as evidenced by an increased fraction of modified amplicons (FIG. 5, left axis, dark grey for MAD7-INLS and light grey representing MAD7-4NLS). Error bars represent one standard deviation for a sample of 3 (n=3). In this experiment, editing frequency was enhanced in Jurkat cells when treated with RNPs comprising MAD-4NLS, which indicates that optimization of the NLS can improve editing efficiency. A slight decrease in cell viability was seen at higher concentrations of RNP for those comprising four NLS as compared to one NLS (FIG. 5, right axis). Specifically, FIG. 5 shows editing frequency at the DNMT1 locus (n=3; Mean±SD) and cell viability of T-cell leukemic cells as a function of MAD7 comprising one or four nuclear localization signal (NLS) and MAD7-RNP amounts (pmol: constant ratio of 1:1.5 MAD7: gNA). Dark grey bars and circles represent mean modification frequency and viability using MAD7-INLS, respectively. Light grey bars and triangles represent mean modification frequency and viability using MAD7-4NLS, respectively.

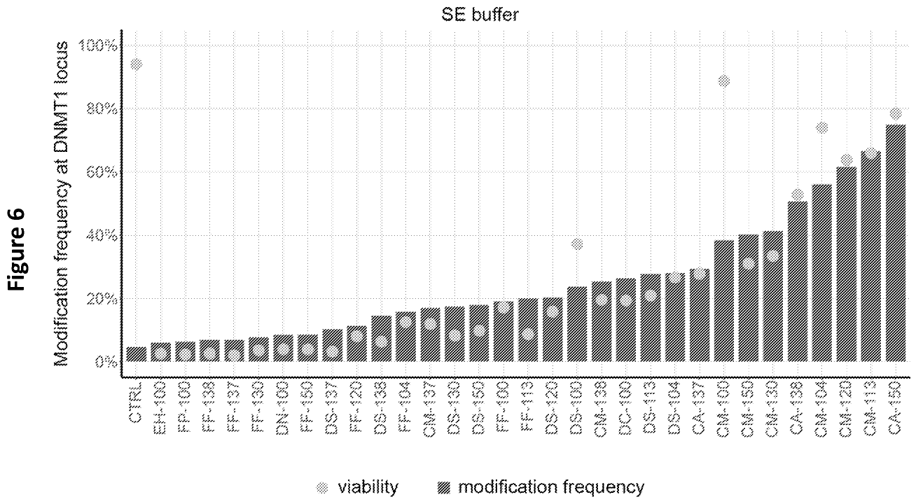

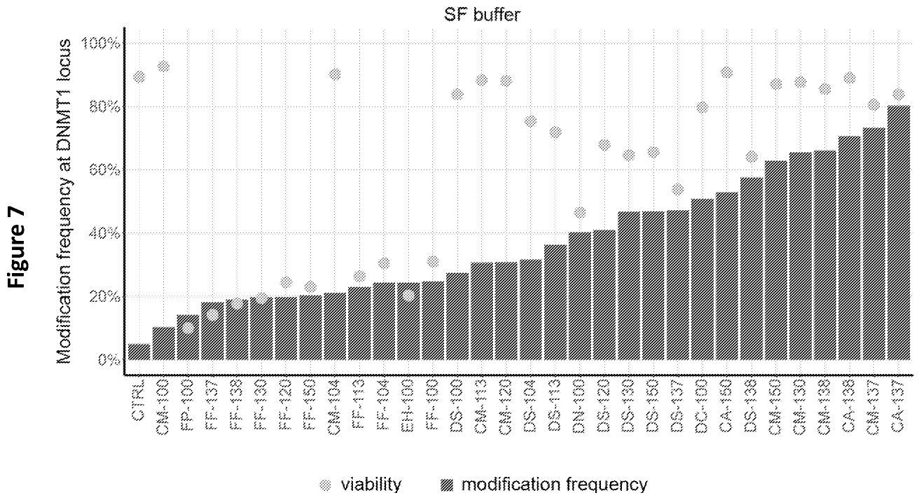

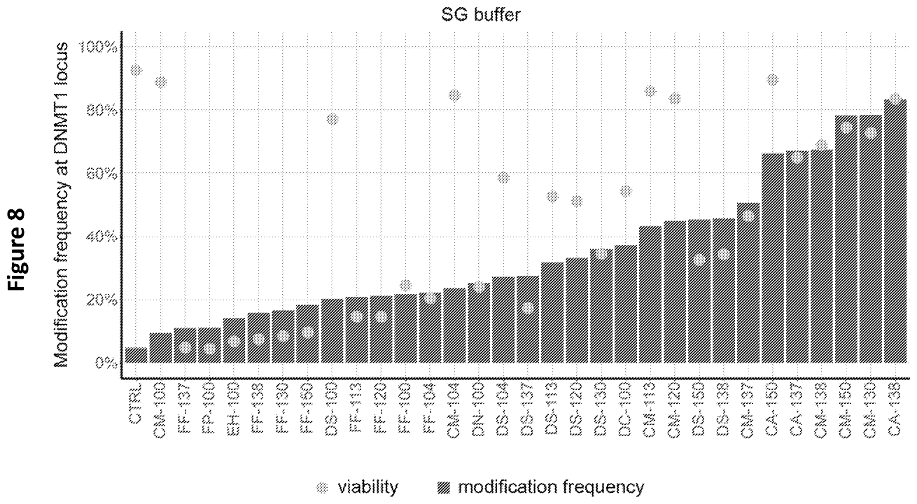

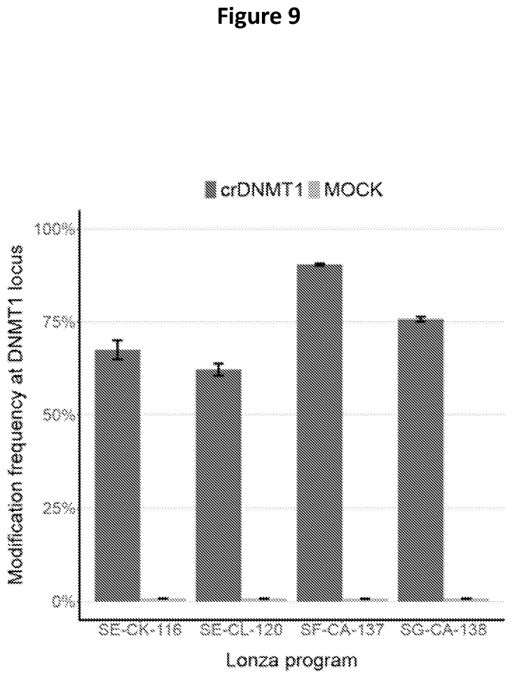

To optimize editing activity, 93 different transfection conditions were tested: 31 nucleofection programs in combination with three buffers-on the Lonza Nucleofector 96-well Shuttle System (FIGS. 6-8). FIGS. 6, 7, and 8 show the editing frequency (bars: x-axis) of each of the electroporation conditions (buffers SE, SF, and SG respectively) as compared to a control (y-axis, control at the top). The majority of buffer-program transfection combinations resulted in suboptimal viability (dots: x-axis) and editing frequency, however, the analysis revealed several conditions that supported substantial rates of both cell viability and editing. Two improved conditions observed in the screen, namely SF-CA-137 and SG-CA-138, were then validated and compared to the Lonza recommended nucleofection programs for T-cell leukemia, namely SE-CL-120 and SE-CK-116 (FIG. 9). Specifically. FIG. 9 shows editing frequency at the DNMT1 locus (n=4; Mean±SD) in T-cell leukemic cell line achieved by utilization of the transfection conditions identified in FIG. 5 (100 pmol MAD7-4NLS) and Lonza recommended nucleofection programs SE-CK-116 and SE-CL-120, as well as the two best nucleofection programs observed in this study. SF-CA-137 and SG-CA-138 (FIGS. 6-8). Dark grey bars represent mean modification frequency using crDNMT1. Light grey bars represent mean modification frequency using crIDTneg (Integrated DNA Technologies, IDT).

### Example 14: Scalable High-Level MAD7-RNP Editing of Immunologically Relevant Genes in Jurkat T-Cell Leukemia Cell Line

The Jurkat T-cell leukemia cell line was used as a model system to screen GNAs demonstrating high editing efficiency. The screen included 298 unique gNAs comprising one or more spacer sequences of SEQ ID NOs: 86-384 of Table 5 targeting the immune checkpoint receptors PDCD1, TIM3, LAG3, TIG1T, and CTLA4, the checkpoint phosphatases PTPN6 (SHP-1) and PTPN11 (SHP-2), and the TCR signaling subunit CD247 (CD35). RNPs were generated as described in Example 5, nucleofected (Example 7), genomic DNA was extracted (Example 10), the edited loci amplified and sequenced (Example 11), and the sequencing data computationally analyzed (Example 12) using the CRISPResso2 algorithm.

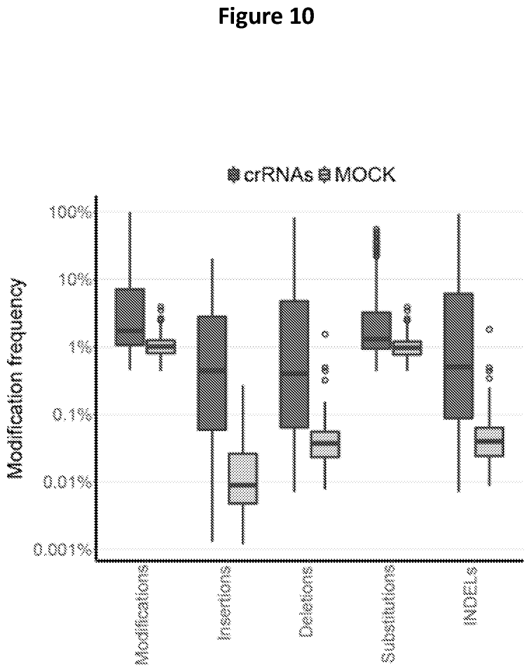

CRISPResso2 software reports the frequency of modifications (insertions, deletions, and substitutions) within a quantification window flanking the position of MAD7-induced cleavage in the amplicon sequence. To better understand detection of editing events, the type of modifications detected in 230 amplicons that were sequenced in both gNA-treated and MOCK samples (no MAD7) were compared. Relatively high modification frequencies (median 1%) in MOCK reactions were observed as a result of high frequency of substitutions (FIG. 10, light grey bars): substitutions were detected at a median frequency of 0.96%, likely due to the errors in NGS base calling or substitutions arising during DNA amplification, while insertions and deletions were found at a much lower median frequency of 0.003% and 0.042%, respectively. Specifically. FIG. 10 shows editing frequency at eight different loci using 298 gNAs (n=3; Mean±SD) in T-cell leukemic cell line as a function of various editing types: all modifications.

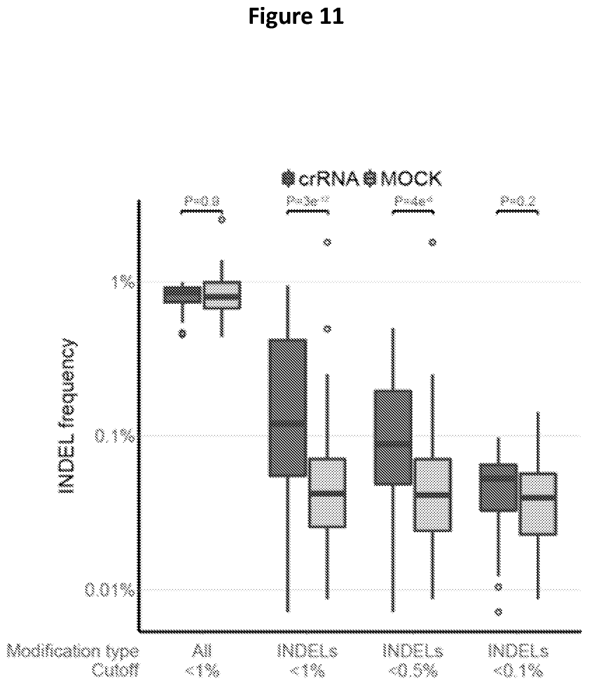

only insertions, only deletions, only substitutions, or insertions and deletions (INDELs). Edits were achieved using the transfection conditions identified in Example 13. FIG. 5 (100 pmol MAD7-4NLS) and one of the tested Lonza nucleofection programs (FIG. 9: SF-CA-137). Dark grey boxplots represent mean modification frequency using gNAs. Light grey boxplots represent mean modification frequency using crIDTneg (IDT). Thus, the frequency of both insertions and deletions (INDEL) were used as a means to quantify the editing activity of the CRISPR-MAD7 system to minimize low end noise. Moreover, low INDEL frequencies in MOCK reactions enabled sensitive detection of editing events at a significantly greater fraction of sites (Fisher exact test, P=3×10⁻¹²: FIG. 11). Analysis of gNAs with low INDEL frequencies showed statistically significant editing in gNA-treated samples compared to MOCK samples at INDEL frequencies as low as 0.5% (Fisher exact test, P=4×10⁻⁸: FIG. 11). This indicates the sensitivity of the assay to detect modifications in the sub-1% range. Specifically, FIG. 11 shows INDEL frequency at eight different loci using 298 gNAs (n=3; Mean±SD) in T-cell leukemic cell line as a function of two modification types: all modifications <1%, and INDELs <1%, or <0.5%, or <0.1%, with lower INDEL frequencies in MOCK compared to gNA reactions at INDELs <1% (Fisher's exact test: P=3×10⁻¹²) and <0.5% (Fisher exact test, P=4×10⁻⁸). Dark grey boxplots represent mean INDEL frequency using gNAs. Light grey boxplots represent mean INDEL frequency using crIDTneg (IDT).

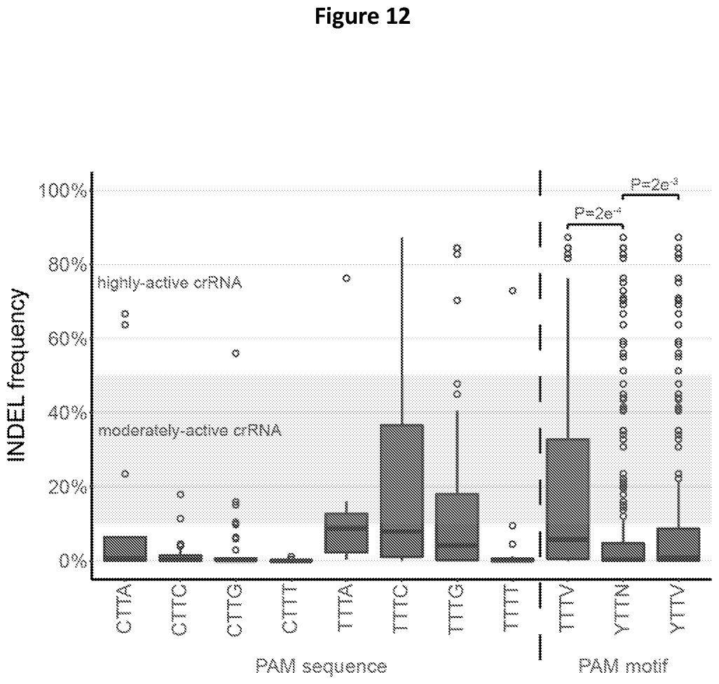

Since MAD7 can target a wide range of PAM, gNAs adjacent to all YTTN PAM variants were screened and editing specificity of MAD7 in Jurkat cells was analyzed. MAD7 demonstrated editing with all eight combinations of YTTN PAM: in this experiment, editing was higher at the YTTV and TTTV consensus sequences (Fisher exact test: P=2×10⁻³ and P=2×10⁻⁴, respectively). While the majority of highly-active (>50% INDEL frequency) gNAs were found at sites with YTTV and TTTV PAMs, moderately-active (>10% INDEL frequency) gNAs were found to target every PAM sequence with the exception of CTTT. This indicates that MAD7 can edit a wide range of target PAMs, albeit at reduced frequencies (FIG. 12). Specifically, FIG. 12 shows INDEL frequency at eight different loci using 298 gNAs (n=3; Mean±SD) in T-cell leukemic cell line as a function of eight YTTN PAM combinations, and TTTV, YTTN, and YTTV PAM motifs. A grey zone on the plot represents moderately-active gNAs (10-50% INDELs), the zone above highly-active gNAs (>50% INDELs), and the zone below active gNAs (1-10% INDELs). INDEL frequency at the YTTV and TTTV PAM motif is significantly higher compared to YTTN motif (Fisher exact test, P=2×10⁻³ and P=2×10⁻⁴, respectively).

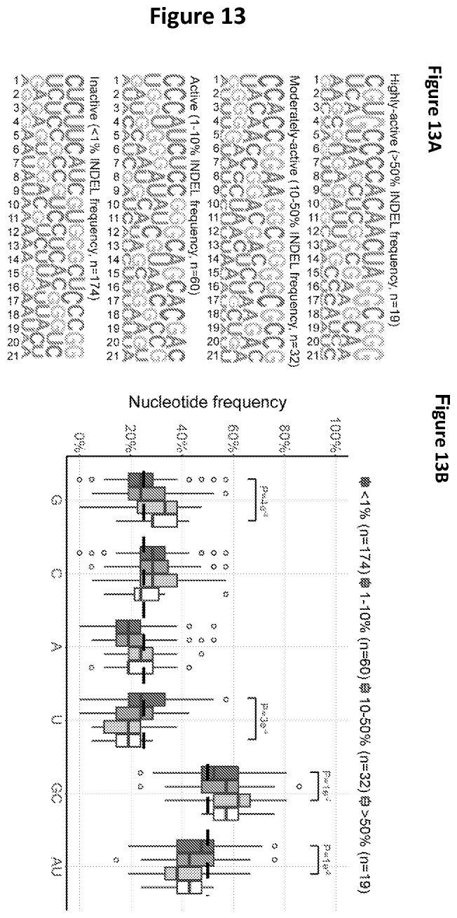

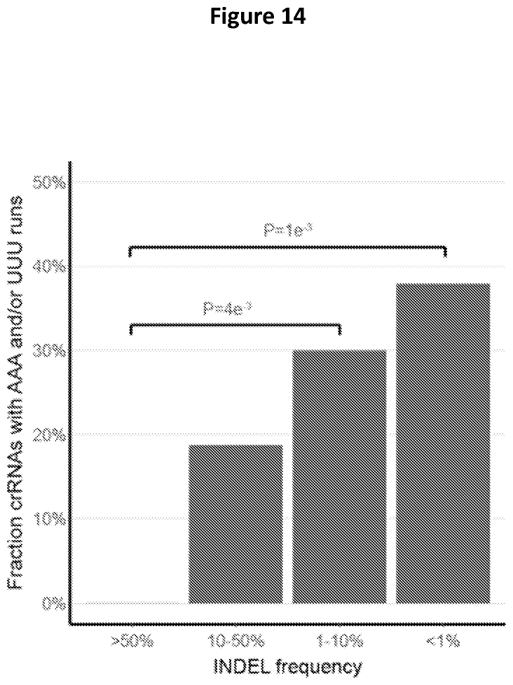

Given the large number of gNAs analyzed, it was determined if the targeted DNA sequence biases editing efficiency. Sequence logos were made to compare the DNA-complementary gNA sequences of inactive (<1% INDELs), active (1-10% INDELs), moderately-active (10-50% INDELs), and highly-active (>50% INDELs) gNAs (FIG. 13A). While there were no strong biases for ribonucleotides at specific positions were identified in this experiment, guanine appeared overrepresented and uracil underrepresented on moderately-active and highly-active gNAs. Next, the frequency of ribonucleotide bases were analyzed within the same four classes of gNAs (FIG. 13B). The analysis confirmed significant enrichment of guanine and depletion of uracil on highly-active gNAs. Specifically, FIG. 13 shows (A) sequence logos comparing DNA-complementary gNA sequences of highly-active (>50% INDELs), moderately-active (10-50% INDELs), active (1-10% INDELs), and inactive (<1% INDELs) gNAs show no strong biases for ribonucleotides at specific positions, however, guanine appeared overrepresented and uracil underrepresented on highly-active and moderately-active gNAs: (B) nucleotide frequency on inactive (<1% INDELs: dark grey box), active (1-10% INDELs: medium grey box), moderately-active (10-50% INDELs: light grey box), and highly-active (>50% INDELs; white box) gNAs, with significant enrichment of guanine and depletion of uracil on highly-active gNAs compared to inactive gNAs (Fisher exact test, P=4×10⁻³ and P=3×10⁻⁴, respectively). Also, significant enrichment of guanine-cytosine content and depletion of adenine-uracil content was observed on moderately-active gNAs compared to inactive gNAs (Fisher exact test, P=1×10⁻²). Moreover, the data showed that nearly 40% of inactive gNAs had runs of three or more adenine or uracil ribonucleotides, while none of the highly-active and <20% of moderately-active gNAs contained such runs (FIG. 14). These sequence features can act as an algorithm for selecting putative high-activity gNAs during initial rounds of screening, and could reduce the overall cost of identifying gNAs for various genes of interest. Specifically. FIG. 14 shows fraction of gNAs with AAA and/or UUU runs as a function of INDEL frequency of highly-active (>50% INDELs), moderately-active (10-50% INDELs), active (1-10% INDELs), and inactive (<1% INDELs) gNAs. Fraction of inactive (<1% INDELs) and active (1-10% INDELs) gNAs containing such runs is higher compared to highly-active (>50% INDELs) gNAs (Fisher exact test, P=1×10⁻³ and P=4×10⁻⁴, respectively).

### Example 15: Validation of gNAs for Gene Editing and Disruption of Immunologically Relevant Genes Using T-Cell Leukemia Cell Line

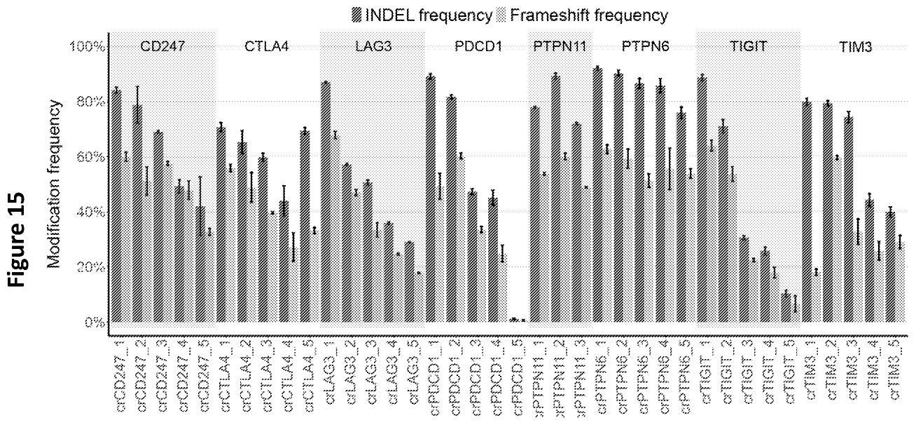

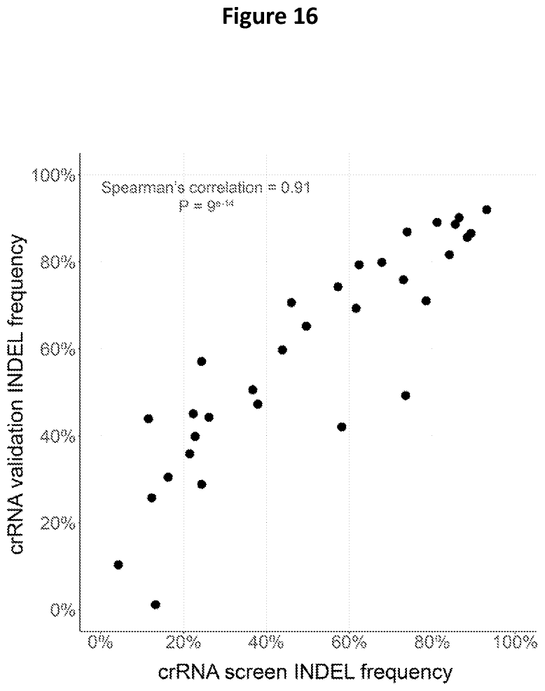

High-efficiency gNAs identified in our initial analysis were validated by assaying INDEL frequency for the top three or five gNAs for each of the selected immunologically relevant genes (FIG. 15). Specifically, FIG. 15 shows INDEL (dark grey bars) and frameshift (light grey bars) frequencies (n=3; Mean±SD) in T-cell leukemic cell line as a function of 38 high-efficiency gNAs. Alternating grey and white zones on the plot represent groups of three to five high-efficiency gNAs per locus. In the validation experiment, the INDEL frequency was significantly correlated to the measurements from the initial screen, highlighting the reproducibility of the INDEL assay (FIG. 16). Specifically, FIG. 16 shows correlation of INDEL frequency in the gNA validation experiment versus INDEL formation in the gNA screen experiment (Spearman's correlation=0.91: P=9×10⁻¹⁴), highlighting reproducibility of the INDEL assay. Using the CRISPresso2 software, the degree of open reading frame (ORF) disruption for each of the validated gNAs was estimated (FIG. 15). In addition, for four high-efficiency gNAs targeting three different exons at the PDCD1 locus, surface expression of the PDCD1 protein was measured by flow cytometry 4, 7, and 11 days post-transfection (data not shown). The data revealed that the protein surface expression after transfection with crPDCD1_2, a gNA targeting the PDCD1 gene at the extracellular domain of the protein, was as low as 10% 4 days post-transfection and remained at this level even at day 11 post-transfection. The surface expression after transfection with the remaining three gNAs was significantly higher, 35% and 85% after transfection with crPDCD1_3 and both crPDCD1_4 and crPDCD1_5, respectively.

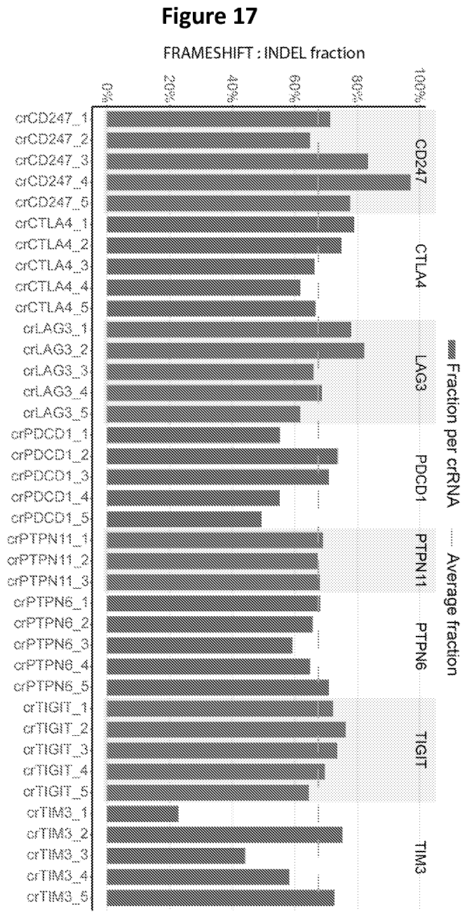

This is in line with the ORF data analysis, which showed that for most of the gNAs including the high-efficiency crPDCD1s, the predicted number of INDELs leading to frameshifts was similar to that expected from an unbiased DNA repair process, with frameshifts in two-thirds of the edited loci (FIG. 17). However, several of the gNAs had a markedly different degree of ORF disruption: crCD247_4 resulted in frameshifts with 97% frequency, while crTIM3_1 and crTIM3_3 resulted in frameshifts with 23% and 44% frequency, respectively (FIG. 17). Specifically, FIG. 17 shows fraction of frameshift to INDEL frequency (dark grey bars) in T-cell leukemic cell line as a function of 38 high-efficiency gNAs. Average fraction of INDELs leading to frameshifts (dashed line) is approx. 66%. Alternating grey and white zones on the plot represent groups of three to five high-efficiency gNAs per locus. The analysis of repair products indicates that in the case of crTIM3_1, and to some extent crTIM3_3, the bias arose from directly repeated sequences at the DNA cleavage site, which possibly promoted microhomology-mediated end joining (MMEJ) repair following DNA cleavage. These data help inform selection of gNAs for gene KO since some gNAs, such as crTIM3_1, have much lower frequency of gene disruption than would be predicted based on the frequency of INDEL formation.

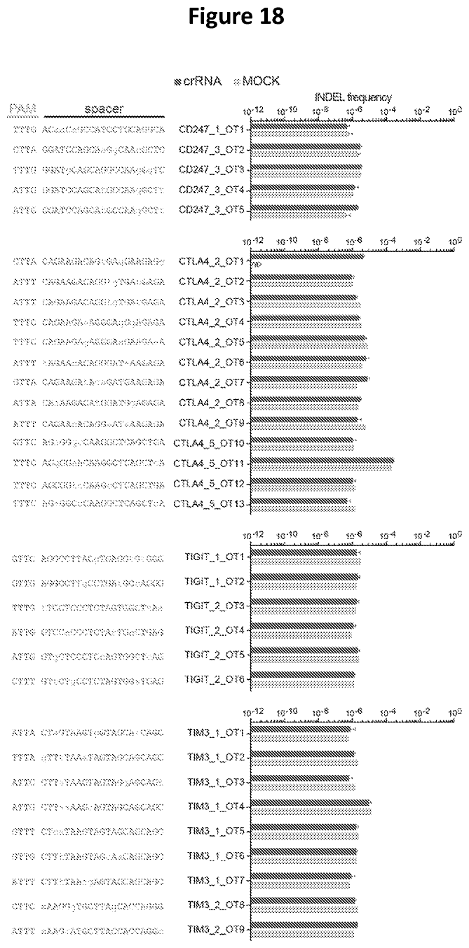

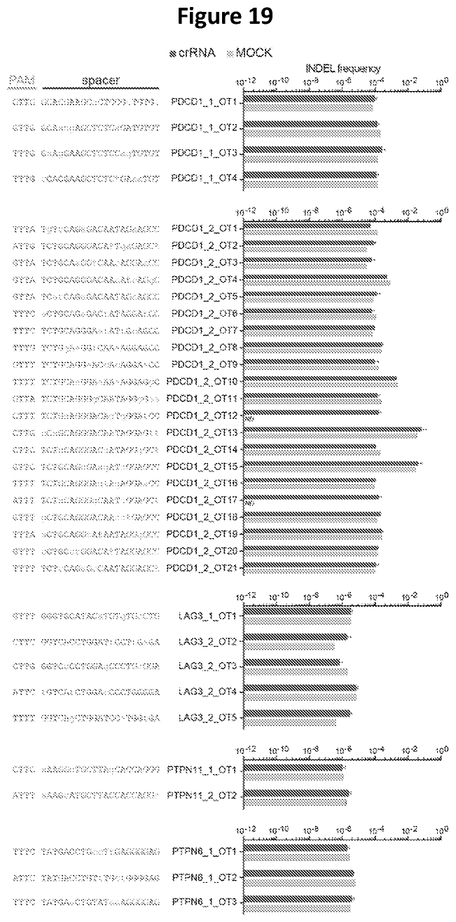

Another consideration for selecting gNAs is the potential for off-target cleavage events. The list of validated gNAs was analyzed using the CasOFFinder software to predict potential off-target editing sites in the genome with up to four mismatches between the gNA and the target DNA sequence. Using the Bioconductor R packages, the predicted off-target sites were matched with the human gene database, and those sites that targeted exons and introns within the genes were extracted. Afterwards, the degree of editing activity at these sites was examined by targeted next-generation sequencing, more specifically, at 25 predicted off-target sites for the top-two PDCD1 gNAs, i.e., crPDCD1_1 and crPDCD1_2. The analysis revealed low-level off-target activity at crPDCD1_2_13 and crPDCD1_2_15 sites, however, INDEL formation at these two sites was statistically insignificant compared to MOCK samples (non-targeting gNAs) (Pairwise T-test, P>0.05; FIGS. 18 and 19). INDEL frequency at 43 putative off-target sites with up to three mismatches between gNA and target DNA sequence were assayed for the top-two gNAs targeting seven remaining genes (i.e., TIM3, LAG3, TIG1T, CTLA4, PTPN6, PTPN11, and CD247; spacer sequences in Table 5). The analysis revealed no detectable activity at any of the putative off-target sites (FIGS. 18 and 19), which confirms the high cleavage fidelity of MAD7-gNA complexes. Specifically. FIGS. 18-19 show INDEL frequency of MAD7 (n=3; Mean±SD) in T-cell leukemic cell line at predicted off-target sites analyzed by targeted deep sequencing. For crPDCD1. INDEL frequency was analyzed at the putative off-target editing sites with ≤4 mismatches between the gNA and target DNA sequence, and with ≤3 mismatches on the remaining gNAs. PAM sequences and spacer sequences with mismatches marked in red are displayed next to their respective measured INDEL frequencies. No significant INDEL frequency at any of the off-target sites was detected (Pairwise T-test, P>0.05).

### Example 16: Transgene Insertion in T-Cell Leukemia Cell Line and Primary T-Cells with CRISPR-MAD7 Platform

Insertion of exogenous transgenes is an important aspect of mammalian cell engineering. Gene insertion with CRISPR-Cas is achieved by homology-directed repair of CRISPR-induced DNA breaks using HDR-donor templates to copy exogenous genetic sequences into targeted DNA loci. Several studies indicate that HDR templates, composed of linear double stranded DNA, provide the most robust and efficient method of transgene insertion using CRISPR-Cas genome editing systems.

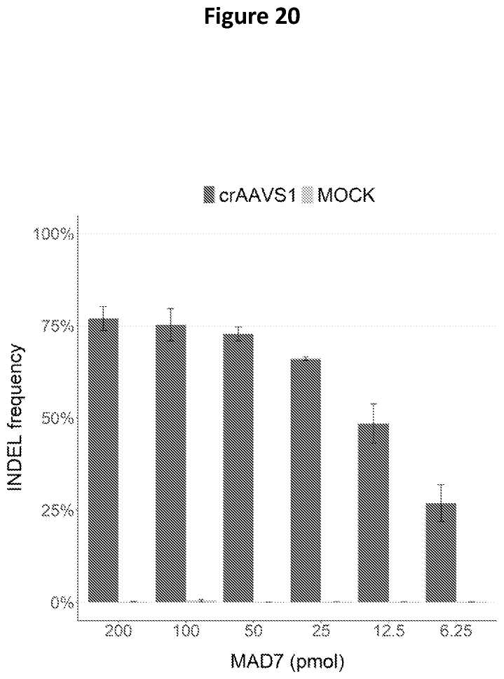

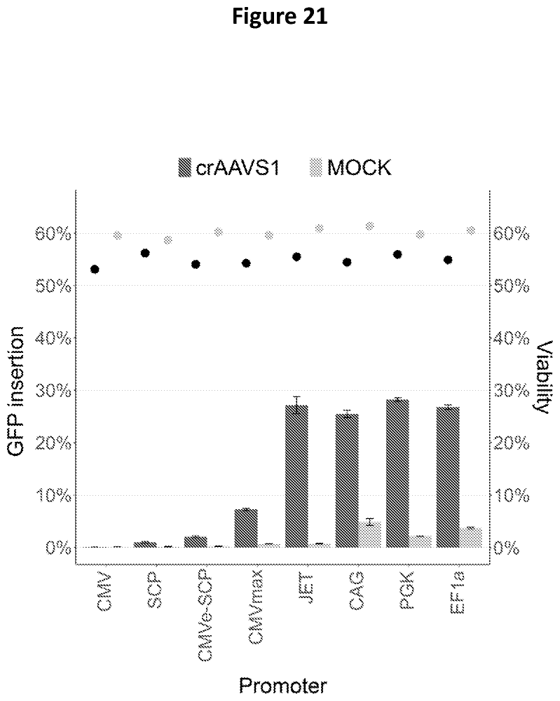

The Jurkat T-cell leukemia cell line was used to evaluate the transgene insertion and expression efficiency using CRISPR-MAD7 RNP complexes. A highly active gNA targeting the AAVSI (spacer sequence in Table 5) safe-harbor locus (FIG. 20) was used in combination with eight different HDR-repair templates flanked with symmetric homology arms (HA) of 500 base pairs (bp) in the amount of 0.5 μg μL⁻¹. Specifically. FIG. 20 shows INDEL frequency at the AAVS1 locus (n=3; Mean±SD) in T-cell leukemic cell line as a function of MAD7-RNP amounts (pmol: constant ratio of 1:1.5 MAD7: gNA). Dark grey bars represent mean INDEL frequency using crAAVS1. Light grey bars represent mean modification frequency using crIDTneg (IDT). The HDR inserts comprised eight promoters (Table 4) differing in both size and promoter strength to drive GFP expression (FIG. 21). When the transient GFP expression diminished at day 14 post-transfection, comparable insertion efficiencies were observed with stable GFP expressions of up to 30% using four (JET, PGK, EF1a, and CAG) out of eight promoters (FIG. 21), suggesting that the insert size has not affected the integration efficiency at AAVSI in human T-cell leukemia cell line. Specifically. FIG. 21 shows GFP insertion efficiency at AAVSI (n=3; Mean±SD) and cell viability of T-cell leukemic cell line measured at day 14 post-transfection. HDR templates consisting of eight different promoters and flanked with symmetric homology arms of 500 base pairs in the amount of 0.5 μg μL⁻¹ were used. Size of promoters in base pairs: CMV, 1400; SCP, 970; CMVe-SCP, 1270; CMVmax, 1830; JET. 1100; CAG, 2600; PGK, 1410; EF-1a, 2090. Dark grey bars and circles present mean insertion frequency and cell viability using crAAVS1. Light grey bars represent mean insertion frequency and cell viability using crIDTneg (IDT).

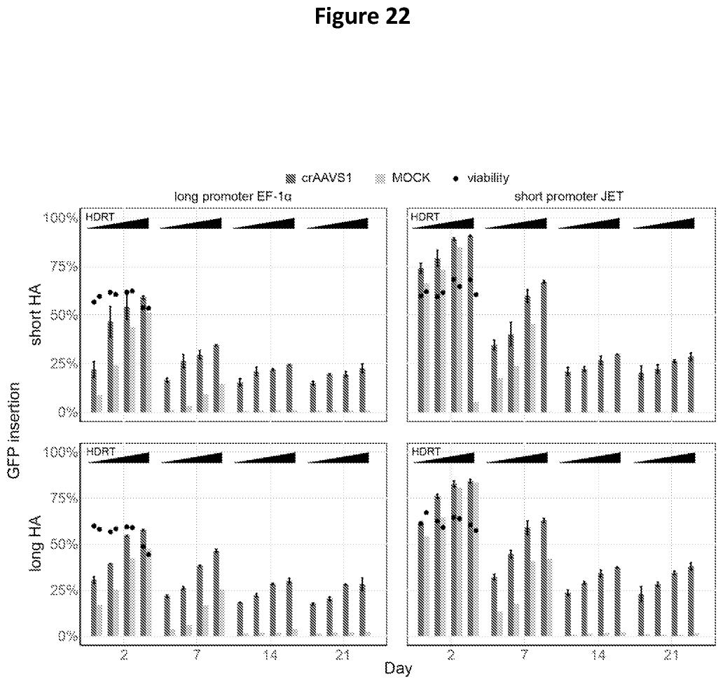

Subsequently, keeping the MAD7-RNP amounts constant, the effect of various homology arm lengths (100 vs 500 bp) and HDR template amounts (0.125 μg μL⁻¹, 0.25 μg μL⁻¹, 0.5 μg μL⁻¹, and 1 μg μL⁻¹) on the insertion efficiency was evaluated using JET and EF1a promoters. Up to 30% higher integration efficiency was observed with HDR templates flanked with HA of 500 compared to 100 base pairs. Moreover, the data showed improved insertion efficiencies with increasing amounts of HDR templates flanked with either 100 or 500 base pair HA but at the same time somewhat reduced cell viability (FIG. 22). Specifically. FIG. 22 shows GFP insertion efficiency at AAVS1 (n=3; Mean±SD) in T-cell leukemic cell line measured at days 2. 7. 14, and 21 post-transfection as a function of donor template amount. No transient GFP expression was observed at day 21 post-transfection. Cell viability (black circles) was measured at day 2 post-transfection. Top panels display GFP insertion efficiencies using donor template flanked with short homology arms (100 bp HA), and bottom panels donor template flanked with long homology arms (500 bp HA). Left panels display GFP insertion efficiencies using donor template containing EF-1a promoter (long, ˜2000 bp), and right panels donor template containing JET promoter (short. ˜1000 bp). Amount of donor template, represented by the gradient above the bars, increases from 0.125, 0.25, 0.5 to 1 μg μL⁻¹. Dark grey bars represent mean insertion frequency using crAAVS1. Light grey bars represent mean insertion frequency using crIDTneg (IDT).

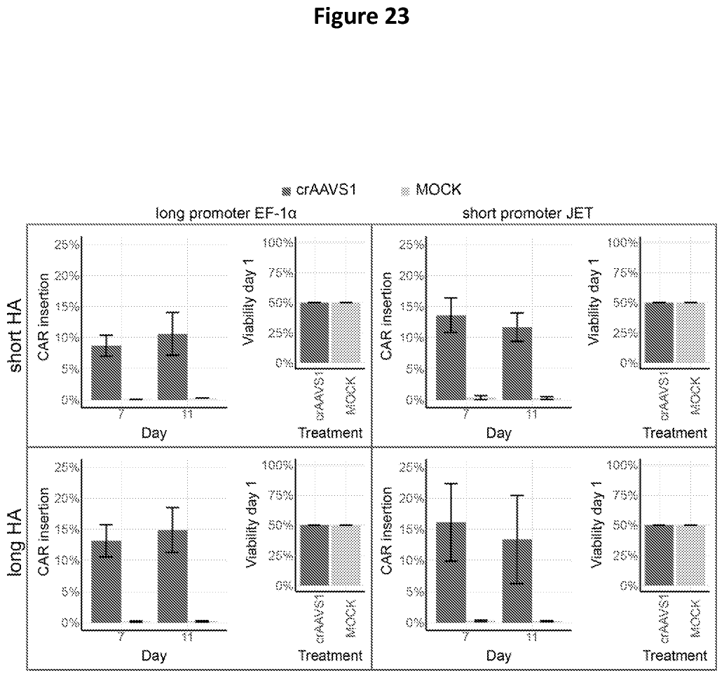

Next, using primary T-cells isolated from the human peripheral blood from three donors and a protocol selected from the experiments above, i.e., 150:100 pmol gNA: MAD7 RNP complex together with 1 μg μL⁻¹ HDR template, in combination with 100 μg μL⁻¹ poly-L-glutamic acid (PGA), integration efficiency of a clinically relevant CAR transgene containing JET or EF1a promoter flanked with HA of 100 or 500 base pairs and a bovine growth hormone derived polyadenylation sequence was analyzed. An anti-CD19 CAR with fully human variable regions (Hu19CAR), CD8a hinge and transmembrane domains, a CD28 costimulatory domain, and CD35 activation domain was used. Moderate insertion efficiency at AAVS1 but stable CAR expression of up to 14% and 16% was observed using HDR templates flanked with 100 and 500 base pair HA, respectively. The normalized cell viability measured 24 h post-transfection was in same cases relatively low, ranging from 22% with JET-500-CAR, 35% with JET-100-CAR, 43% with EF1a-100-CAR, to 55% with EF1a-500-CAR (FIG. 23). It is important to emphasize, that both CAR insertion efficiency and cell viability were higher in the treatment with PGA compared to the treatment without PGA (P≤0.05; data not shown). Specifically, FIG. 23 shows CAR insertion efficiency at AAVS1 (D=3; n=3; Mean±SD) in primary Pan T-cells measured at days 7 and 11 post-transfection. Cell viability was measured 24 hours post-transfection. Individual panels display CAR insertion efficiencies using donor template structure as described in FIG. 22. Amount of donor template, MAD7-RNP, and PGA was 1 μg μL⁻¹, 100:150 pmol MAD7: gNA, and 100 μg μL⁻¹, in that order. Nucleofection program P3-EH-115 for transfection of primary T-cells was used. D represents number of biological replicas, and n number of technical replicas per D. Dark grey bars represent mean insertion frequency using crAAVS1. Light grey bars represent mean insertion frequency using crIDTneg (IDT).

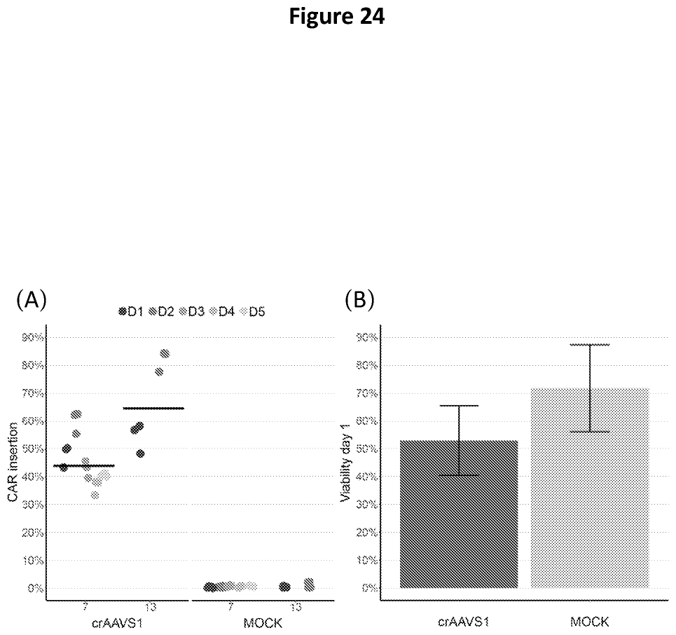

Multiple parameters were reevaluated to further optimize primary T-cell viability and CAR insertion efficiencies at AAVS1. Using Pan T-cells isolated from the blood from two donors, the effect of RNP amount with 100 μg μL⁻¹ PGA and EF1a-500-CAR template amount on CAR insertion efficiency and cell viability was tested (data not shown). Reducing the RNP amount to 75:50 pmol gNA: MAD7 RNP complex while increasing the donor template amount to 1.5 μg μL⁻¹ led to improved CAR insertion efficiencies without significantly affecting cell viability (P≥0.05; data not shown). In addition, using the abovementioned transfection conditions in combination with the cell recovery in a post-transfection cultivation medium pretreated with 2 μM M3814 resulted in nearly 5-times more efficient CAR insertion than other experiments (FIG. 24). The optimized CRISPR-MAD7 transfection protocol resulted in CAR insertion efficiency of up to 85% 13-days post-transfection (median 65%) together with the median normalized cell viability as high as 62% 24 hours post-transfection. Specifically, FIG. 24 shows CAR insertion efficiency at AAVS1 (D=5; n=3) in primary Pan T-cells measured at day 7 post-transfection, and re-measured in two biological replicas at day 13 post-transfection (D=2; n=3). Cell viability was measured 24 hours post-transfection (D=5; n=3; Mean±SD). Amount or concentration of donor template, MAD7-RNP, PGA, and M3814 was 1.5 μg μL⁻¹, 50:75 pmol MAD7: gNA, 100 μg μL⁻¹, and 2 μM, respectively. Nucleofection program P3-EH-115 for transfection of primary T-cells was used. D represents number of biological replicas, and n number of technical replicas per D. Dark grey bars represent mean insertion frequency using crAAVS1. Light grey bars represent mean insertion frequency using crIDTneg (IDT).

### Example 17

gRNAs were designed on Benchling using the standard CRISPR tool. All synthetic As. Cas12a gRNAs were ordered from IDT. The synthetic gRNAs were ordered in two different configurations: regular and dual gRNA design. The regular gRNA design was used for the selection of top gRNAs, and the top gRNAs were tested in the regular and dual gRNA design. dual gRNAs consisted of two parts instead of one: the modulator (crRNA) and the targeter (including spacer sequence) part.

Jurkat cells were used for tiling experiments, and for optimization and verification of the top gRNAs. The cells were maintained by splitting every 2-3 days. Briefly, materials were sterilized before use in a Biosafety Cabinet Class II (BSC): 15 mL and 50 ml conical tubes, serological pipettes, pipet filler/Pipetboy, RPMI media, FBS, culture flask, pipette tips. Culture media RPMI with 10% FBS was prepared in the BSC and pre-warmed to 37° C., in a water/armor beads bath. In the BSC, 9 mL of pre-warmed cell culture media was added to a 15 mL conic tube. A Styrofoam box was filled with dry ice and the frozen vial(s) of Jurkat cells were removed from from liquid N₂ storage and placed in dry ice. The vials were then placed in a 37° C., water bath to thaw, while avoiding water from contacting the screw cap. Once the cells were thawed, the vial was sprayed with 70% ethanol and brought into the BSC. Note: Work fast because DMSO is toxic to the cells at room temperature. A 1 mL micropipet and tips were used to transfer the whole contents in the cryovial (1 mL) into the falcon tube containing 9 mL of media in a drop-wise manner, then centrifuged at 300×g for 5 min at RT. The supernatant was discarded and the pellet resuspended in 5 ml cell culture media. The viable cell density (cells/ml) and viability (%) was determined using the NucleocounterTM NC202 according to the owner's manual. The cell culture volume was adjusted so that the cell density was 2E5 cells/mL. Cultures were mainted at 2E5 cells/ml and counted every 2-3 days (e.g., Monday-Wednesday-Friday-Monday) An aliquot was transferred to a new flask and dilute with pre-warmed full media (RPMI+10% FBS) to 2E5 cells/ml. On the day before transfection cells were seeded to 1E5 cells/mL.

T-cells were isolated to form buffy coats and nucleofected after two days. The buffy coats were procured from the Rigshospitalet in Copenhagen, Denmark. The Pan-T cells were enriched by negative selection using an Easy Sep Human T-cell Isolation Kit from StemCell Technologies. The RNPs were formed by mixing gRNAs with Art-Mad7mam+ prior to nucleofection with Jurkat or T-cells. In some cases, synthetic ssODNs (Table 11) with a length of 200 nt were included in the transfections to evaluate impact on frame shift mutation rates by HDR rather than relying on NHEJ alone in an effort to maximize functional disruption. The ssODNs were designed to comprise a deletion of the spacer and PAM sequence thereby resulting in a programmed frame shift. After transfection, the cells were cultivated and assayed for the different readouts. The nucleofection with dual gRNAs followed a similar protocol, but with minor differences in pre-assembling of the gRNAs. Briefly, the two dual RNAs (modulator and targeter) are combined in a molecular ratio of 1:1 (400 μM Modulator

(5′/AltR1/UAAUUUCUACUC 3′) + 400 uM Targeter
(Targeter_CSF2_007: 5′
UUGUAGAUCACAGGAGCCGACCUGCCUAC/AltR2/3′;
Targeter_TRBC1_2_003: 5′
UUGUAGAUAGCCAUCAGAAGCAGAGAUCU/AltR2/3′;
Targeter_CD3E_24: 5′
UUGUAGAUAGAUCCAGGAUACUGAGGGCA/AltR2/3′;
Targeter_CD40LG_40: 5′
UUGUAGAU CUGCUGGCCUCACUUAUGACA/AltR2/ 3′))
at RT for 15 min.

Cell viability was measured by two different methods depending on the sample number to be measured. For 1-10 samples, the nucleocounter was used. The measurement of the Nuclecounter Via2 cassette is based on Acridine Orange and DAPI staining of the cells whereby Acridine Orange and DAPI double positive cells were counted as dead cells and Acridine orange positive/DAPI negative cells were counted as viable cells. High-throughput microscopy using the Image Xpress pico device (IXP assay) was used when more than 10 samples were measured in 96-well plate format. The measurement of the IXP assay is based on Hoechst and Propidium iodide staining whereby Hoechst stains all cells and Propidium iodide the dead cell population. The assay was performed in 96-well plates and analyzed with the Image Xpress pico high-throughput microscope.

At the genome level, Amplicon-NGS was used to determine the type and mutation frequency at the on-target site. The Amplicon-NGS assay is based on the amplification of the on-target site using specific primer pairs. The primers are designed to have the predicted cutting site at the center and a length of 180-280 nt. The amplicons are indexed for NGS and sequenced on an Illumina MiSeq system. The analysis was performed using the Crispresso script. Crispresso requires the input of a configuration file comprised of the specific gRNA sequence, primer sequences and the theoretical amplicon that is based on the alignment of the primer pair to the reference human genome GRCh38. In addition, for HDR analysis associated with ssODN-programmable editing a reference sequence with the specific mutations can be input into the configuration file. Crispresso aligns the sequencing reads to the reference amplicon and analyzes the mutation type and frequency −/+10 nt at the putative cut site of Art-Mad7mam+ based on the provided gRNA sequence. As output, Crispresso delivers different mutation types and their frequencies: Substitutions, INDELs, HDR Unmodified, HDR INDELs, HDR+substitutions.

Protein expression for targeted genes was measured by antibody staining against the target protein to quantify functional disruption of the target gene. The efficiency of functional disruption was verified by antibody staining and quantified as the negative cell population by flow cytometry. TRBC, CD3E and CD40LG are located at the cell surface and living cells were stained with the corresponding antibodies. Since CSF2 is a secreted protein, cells were first activated, secretion was blocked, cells were fixed with formaldehyde, and permeabilized before the staining procedure. Given CD40LG and CSF2 are expressed upon stimulation of primary T-cells, cells were activated first to enhance their expression. The induction of CD40LG and CSF2 was achieved using anti-CD3/CD28 and PMA/Ionomycin, respectively.

Predicted off-target sites were identified using the online tool CCTop. The putative off-target sites were ranked based on the following criteria: 1) similarity to the MAD7 PAM sequence: 2) the number of mismatches in the spacer sequence: 3) location in the genome (exon, intron or intergenic). The identified putative off-target sites were next evaluated to determine actual in-cell editing using rhAMP-seq, a similar approach to Amplicon-NGS. rhAMP-seq technology uses multiplex PCR to amplify the different genomic sites (producing amplicons) in combination with NGS. The sequencing reads were analyzed using Crispresso as described for above in the Amplicon-NGS section.

The cutting efficiency was calculated as the percentage of reads with any NHEJ modification (including insertions, deletions, and/or substitutions) of the total number of reads that were aligned to the reference amplicon. Editing with the 176 gRNAs results in a wide range of efficiencies (0 to 90%) for the five targets. Summary of results are shown in Table 13.

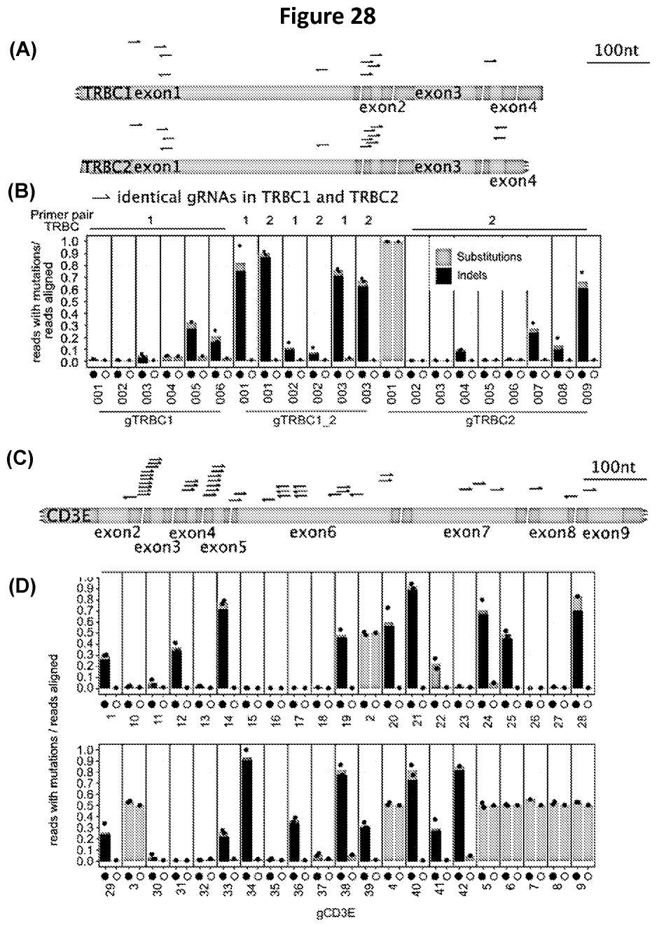

TRBC1 and TRBC 2. It is known that TCR beta is encoded by two genes, TRBC1 and TRBC2. Fortunately, the sequences for TRBC1 and TRBC2 exon I were nearly identical enabling the design of four gRNAs with identical spacer and PAM sequence that target both genes simultaneously (FIG. 28A). Specifically, FIG. 28A shows a schematic overview of the protein coding exons of TRBC1 and TRBC2 and the location of the designed gRNAs (black arrows). Fifteen additional gRNA's were designed and evaluated for individual disruption of TRBC1 and TRBC2 in addition to the four overlapping guides. After evaluating all 19 gRNA's, two gRNAs targeting both TRBC1 and TRBC2 demonstrated >60% cutting efficiency (FIG. 28B). Specifically, FIG. 28B shows tiling results of the TRBC gRNAs (x-axis) with the resulting INDEL and Substitution frequencies (y-axis). Some gRNAs were analyzed with two different primer sets, and these are marked with a ‘l’ or ‘2’ in the top of the panel.

CD3E. 42 gRNA's were designed and synthesized as part of the initial panel. 9 gRNA's were characterized with a cutting efficiency of higher than 60% (FIGS. 28C and 1D). Specifically, FIG. 28C shows a schematic overview of the protein coding exons of CD3E and the location of the designed gRNAs (black arrows). We identified 7 gRNAs with ˜50% substitutions in the experimental and control sample. These regions most likely contain a SNP in the spacer or its approximate region (−/+10 nt from the cut site). Specifically, FIG. 28D shows tiling results of the CD3E gRNAs (x-axis) with the resulting INDEL and substitution frequencies (y-axis). Black filled circles represent cells treated with RNPs, empty circle samples are controls where wildtype Jurkat genomic DNA for Amplicon-NGS was used.

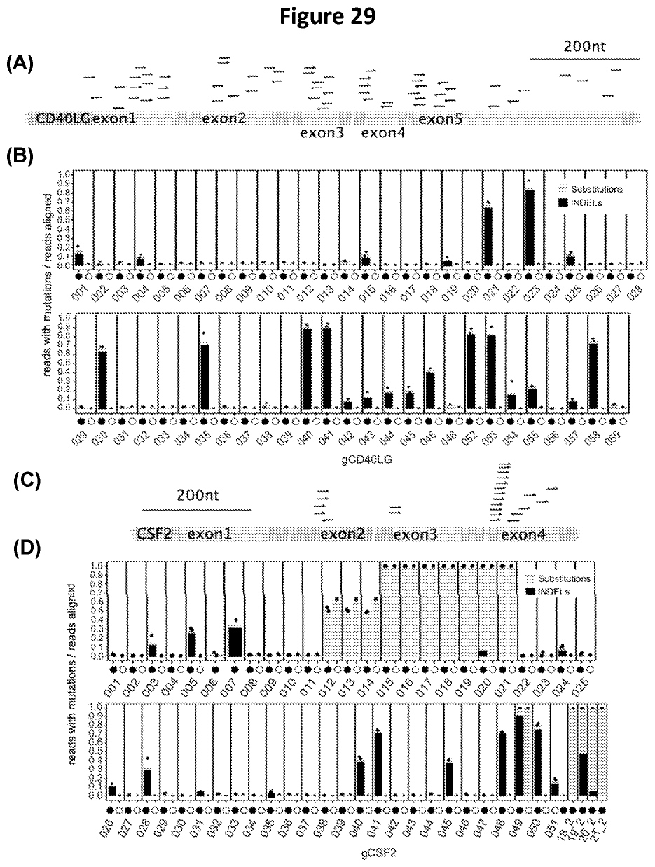

CD40LG. 60 gRNAs were designed to target CD40LG (FIG. 29A). Specifically, FIG. 29A shows a schematic overview of the protein coding exons of CD40LG and the location of the designed gRNAs (black arrows). Initial evaluation of the full panel of gRNAs revealed nine gRNA candidates with a cutting efficiency that was higher than 60% (FIG. 29B). Specifically, FIG. 29B shows tiling results of the CD40LG gRNAs (x-axis) with the resulting INDEL and substitution frequencies (y-axis).

CSF2: 25 gRNAs were potentially active in the protein-coding exon of CSF2 (FIG. 29C). Specifically, FIG. 29C shows a schematic overview of the protein coding exons of CSF2 and the location of the designed gRNAs (black lines). After initial evaluation of editing efficiency, three gRNAs resulted in low to moderate cutting efficiency of 10-30% (FIG. 29D). Specifically, FIG. 29D shows tiling results of the CSF2 gRNAs (x-axis) with the resulting INDEL and Substitution frequencies (y-axis). Black filled circles represent cells treated with RNPs, empty circle samples are controls and wildtype Jurkat genomic DNA for Amplicon-NGS was used. In addition, we observed that three gRNAs had potential heterozygous SNPs in the spacer and/or surrounding region of the spacer, and an additional seven gRNAs had potential homozygous SNPs. The genome of Jurkat cells is available (Gioia et al. 2018) and revealed indeed two SNPs in this region. In addition, three gRNA target sequences were affected by the SNPs. To screen for additional gRNAs with a cutting efficiency >60%, we redesigned the four gRNAs with the observed SNP's in addition to 26 gRNAs that targeted CSF2 introns. Six gRNAs showed a cutting efficiency >40% in the second pass evaluation and were promoted for additional characterization and optimization.

TABLE 13
Cutting efficiencies and off-target scores for selected gRNAs

| | | Average | | |
|---|---|---|---|---|
| | | on-target | Off-target | |
| | | editing | score | Total |
| Index | gRNA_name | efficiency | (predicted) | score |
| 1 | gCSF2_007 | 31.1 | 98.8 | 30.7 |
| 2 | gCSF2_005 | 27.8 | 77.1 | 21.4 |
| 3 | gCSF2_003 | 14.2 | 75.6 | 10.7 |
| 4 | gCD40LG_041 | 89.3 | 95.8 | 85.5 |
| 5 | gCD40LG_040 | 88.9 | 95.3 | 84.7 |
| 6 | gCD40LG_052 | 81.7 | 95.8 | 78.3 |
| 7 | gCD40LG_053 | 80.1 | 95.0 | 76.1 |
| 8 | gCD40LG_023 | 84.1 | 89.8 | 75.5 |
| 9 | gCD40LG_058 | 72.1 | 96.7 | 69.7 |
| 10 | gCD40LG_035 | 69.9 | 90.6 | 63.3 |
| 11 | gCD40LG_021 | 64.3 | 90.8 | 58.4 |
| 12 | gCD40LG_030 | 61.7 | 78.1 | 48.2 |
| 13 | gCD40LG_046 | 40.1 | 94.5 | 37.9 |
| 14 | gCD3E_34 | 92.3 | 94.9 | 87.6 |
| 15 | gCD3E_21 | 88.9 | 94.2 | 83.8 |
| 16 | gCD3E_42 | 80.8 | 97.7 | 78.9 |
| 17 | gCD3E_38 | 78.2 | 90.0 | 70.4 |
| 18 | gCD3E_40 | 72.6 | 92.5 | 67.2 |
| 19 | gCD3E_24 | 68.0 | 96.8 | 65.8 |
| 20 | gCD3E_20 | 56.7 | 91.6 | 51.9 |
| 21 | gCD3E_19 | 46.0 | 90.2 | 41.5 |
| 22 | gCD3E_14 | 71.6 | 56.4 | 40.4 |

The top gRNAs identified in the tiling experiments in, were further tested by performing additional nucleofections in Jurkat cells and measuring viability, INDEL formation by amplicon-NGS, and functional flow cytometry assays. These nucleofections were performed with two TRBC gRNAs and the CD3E, CD40LG and CSF2 gRNAs (Table 13). For the Amplicon-NGS readout three days after transfection, genomic DNA was prepared, amplicons generated, and sequence analyzed. Functional KO verification was performed with antibody staining for TRBC and CD3E, but not for CD40LG and CSF2, CD40LG and CSF2 are expressed upon activation of T-cells and will be tested for functional KOs in Pan T-cells. In this experiment, ssODNs for TRBC and CSF2 gRNAs (Table 11) were included to induce directed loss of function mutations at the on-target site. The ssODNs are 200 nt in length, centered at the on-target site and contains a 25 nt deletion (PAM+spacer sequence) in the center of the ssODN to force a frame shift upon integration by homology directed repair at the target site.

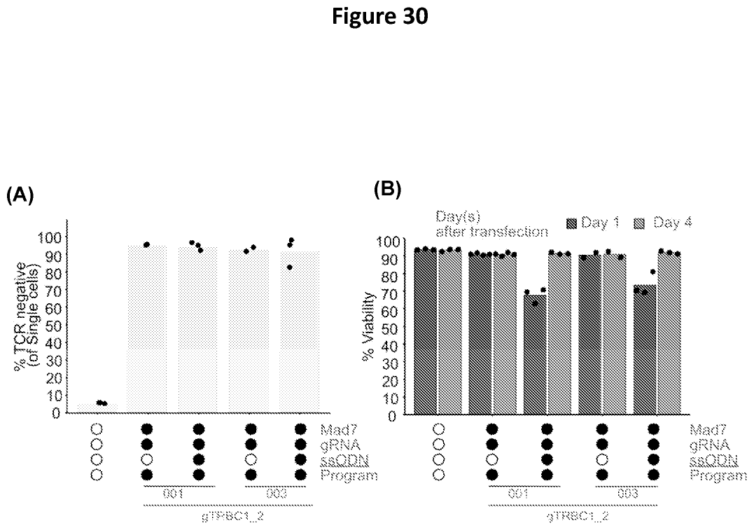

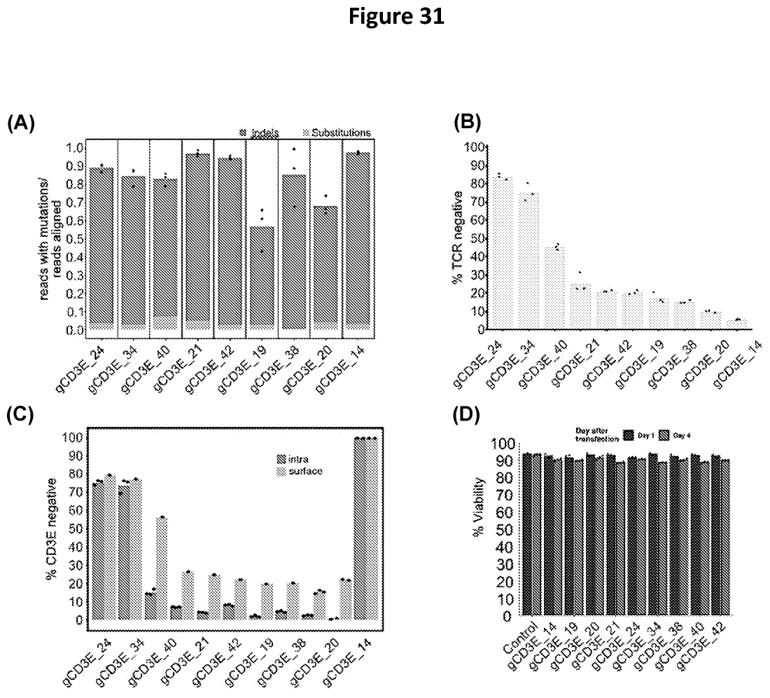

TRBC1 and TRBC2 serves as the beta chain of the TCR complex that are located at the surface of the cells, CD3E is present with two subunits in the TCR complex. We leveraged the anti-TCR antibody staining followed by flow cytometry to verify the KO efficiency of the TRBC1 and TRBC2 proteins and the anti-TCR and CD3E antibody for verification of CD3E KO. In addition, CD3E intracellular levels were measured to compare and understand the effectivity of CD3E KO and TCR surface expression because CD3E partial KO might abolish TCR localization to the surface and TCR surface expression as marker might overestimate the CD3E KO efficiency. The Jurkat cells treated with the different gCD3E and gTRBC1/2/RNPs were stained with antibodies for the relevant proteins and analyzed using flow cytometry. The flow cytometry data were gated for viable, single, and TCR or CD3E negative cells and the data of the replicates were summarized as bar plots (TRBC1/2: FIG. 30A: CD3E; FIGS. 31B and C). Specifically, FIG. 30A shows TCR staining results (TCR negative cells on y-axis) after transfection of TRBC1 and TRBC2 RNPs and the control (x-axis): FIGS. 31B and C show TCR and CD3E staining results (TCR and CD3E negative cells on y-axis respectively) after transfection of gCD3E RNPs and the controls (x-axis). Performing nucleofection with the gTRBC1_2_001 and gTRBC1_2_003 RNPs resulted in an increase to more than 90% TCR negative cells of the population. The addition of ssODNs did not increase the negative population (FIG. 30A). The viability of the treated cells are shown in FIGS. 30B and 31D for TRBC1/2 and CD3E respectively.

Transfection of gCD3E_24 and gCD3E_34 resulted in ˜85 and ˜75% TCR negative cells, respectively. Transfection with all other gRNAs resulted in less than 50% TCR negative cells (FIG. 31B). Similar, CD3E surface staining resulted in a comparable negative cell population as observed for TCR (FIG. 31C). The intracellular CD3E amount was slightly higher relative to CD3E surface amount (FIG. 31C). However, the CD3E or TCR surface staining can be used to determine the KO efficiency of the CD3E RNP-transfected cells. Amplicon-NGS verified that the cutting efficiency of the different gRNAs was above 50% (FIG. 31A) as observed before, but only the cutting rate of gCD3E_24 and gCD3E_34 correlate with their functional KO efficiency. Interestingly, both gRNAs were targeting the protein-coding exon 6 and exon 6 might be the optimal CRISPR target to obtain functional KO of CD3E. The viability of the cells treated with the different RNPs were above 85% (FIG. 31D).

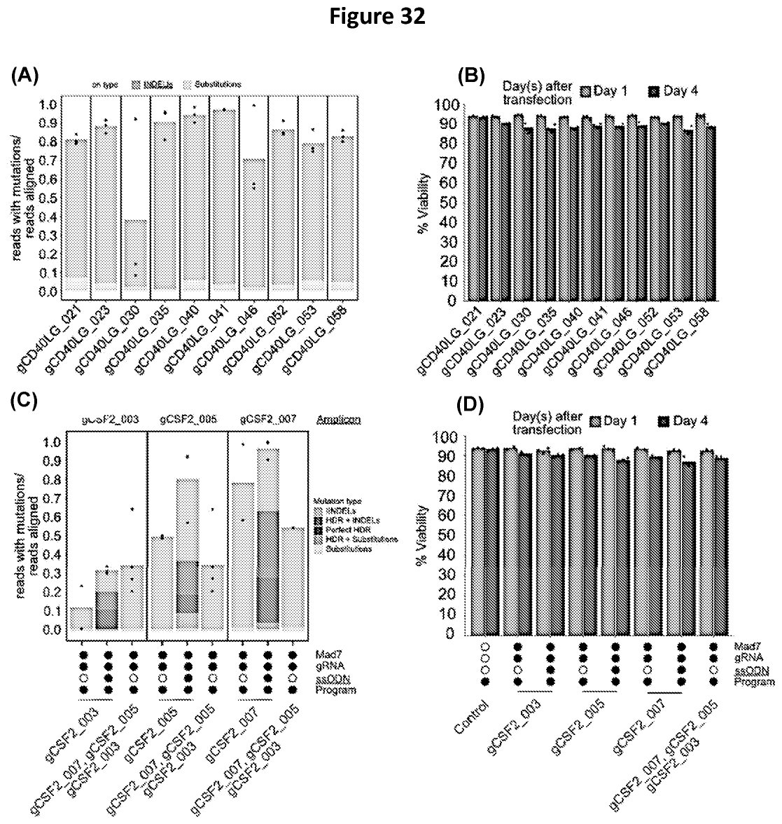

Nine CD40LG gRNAs in complex with Art-Mad7mam+ were further tested and the cutting efficiency and their impact of the viability after transfection in Jurkat cells was assessed. All tested gRNAs except gCD40LG_030 were verified as strong cutters (FIG. 32A) when compared to the tiling experiment and no obvious impact on viability after transfection was observed (FIG. 32B).

Three gRNAs with moderate cutting efficiency based on Amplicon-NGS analysis were further optimized and tested. To optimize, ssODNs were designed for all three gRNAs to direct mutagenesis via HDR and increase KO efficiency. In addition, all three gRNAs were combined in a single transfection in an effort to maximize editing. We observed a similar efficiency pattern to that detected in the initial tiling experiment in that gCSF2_3 and gCSF2_7 showed the lowest and highest cutting efficiency respectively. The inclusion of ssODNs in the transfection further increased the mutation rate by ˜1.5 fold in all three cases and no toxicity effect were observed after electroporation (FIGS. 32C and D respectively).

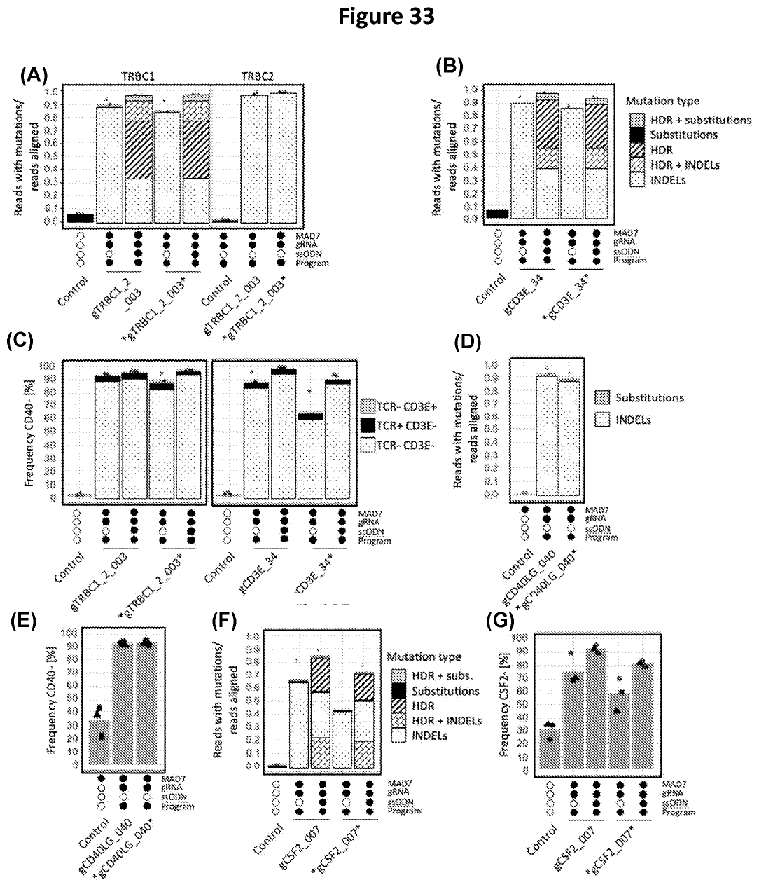

To further test the effects of ssODNs, single and dual gRNAs were mixed with Art-MAD7mam+ nuclease, and in some cases included a ssODN designed to create a programmed disruption in the target gene. The freshly isolated Pan-T cells were activated and cultured for 2 days, and RNPs−/+ssODNs were transfected using the Lonza 96-well plate shuttle. Following transfection, samples were obtained at day 3 after electroporation for gDNA extraction and NGS verification of cutting/editing and off-target analysis using rhAmp-seq at day 6, followed by flow cytometry analysis of functional disruption of the TCR, CD3E and CD40LG and CSF2, respectively, and a time course was taken to evaluate viability. Functional disruption of TCR in Pan T-cells was achieved by transfecting RNPs with gTRBC1_2_003 and gCD3E_34. In both cases, the INDEL frequency was ˜90% at the on-target site using the regular gRNA configuration (FIGS. 33A and B). The dual configuration resulted in ˜5% lower INDEL frequency in case of TRBC1 and TRBC2 and a stronger reduction of ˜15% for CD3E. The inclusion of ssODNs increased the perfect HDR rate to ˜40% at the genomic level and increased the mutation rate in all cases >90%. The functional KO rates for both targets were like the mutation rates observed with Amplicon-NGS readout and resulted in >90% negative TCR/CD3E cell population for the regular and dual gRNAs with ssODNs (FIG. 33C).

For gCD40LG_40, ˜90% mutation rates were detected with both the regular and the dual gRNA configuration (FIG. 33D), CD40LG is expressed at the cell surface upon activation of Pan T-cells. Therefore, the Pan T-cells were activated with CD3/CD28 for 6 hours prior to anti-CD40LG staining and ˜70% of the Pan T-cells expressed CD40LG at the surface. Despite activation, around 90% of the cell population that were transfected with RNPs remained CD40LG negative (FIG. 33D).

The best CSF2 gRNA was identified as gCSF2_007 in the Jurkat experiment. In Pan T-cells, the cutting efficiency was approximately 65% and 45% and could be increased by ssODN inclusion to ˜80 and 70% for the regular and STAR design, respectively (FIG. 33E). CSF2 is a secreted protein and is expressed and upregulated in activated cells. The cells were treated with PMA and Ionomycin to strongly activate the cells and blocked the secretion of the protein with Golgiplug and Golgistop and stained the cells after fixation and permeabilization for the intracellular accumulated CSF2 proteins. This process resulted in 70% of the cell population positive for CSF2 in the control cells without RNP transfection. CSF2 gRNA treatment increased the negative CSF2 cell population to ˜75% and ˜60% and were further increased with the ssODN to ˜90% and 80% for regular and dual gRNA, respectively (FIG. 33G).

### X. Equivalents

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present invention, whether explicit or implicit herein. For example, where reference is made to a particular compound, that compound can be used in various embodiments of compositions of the present invention and/or in methods of the present invention, unless otherwise understood from the context. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

The terms “a” and “an” and “the” and similar references in the context of describing the invention (especially in the context of the following claims) are to be construed to cover both the singular and the plural, unless otherwise indicated herein or clearly contradicted by context. For example, the term “a cell” includes a plurality of cells, including mixtures thereof. Where the plural form is used for compounds, salts, or the like, this is taken to mean also a single compound, salt, or the like.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present invention also includes the specific quantitative value itself, unless specifically stated otherwise. As used herein, the term “about” refers to a +10% variation from the nominal value unless otherwise indicated or inferred.

It should be understood that the order of steps or order for performing certain actions is immaterial so long as the present invention remain operable. Moreover, two or more steps or actions may be conducted simultaneously.

The use of any and all examples, or exemplary language herein, for example, “such as” or “including,” is intended merely to illustrate better the present invention and does not pose a limitation on the scope of the invention unless claimed. No language in the specification should be construed as indicating any non-claimed element as essential to the practice of the present invention.

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

1. A composition comprising a plurality of ssODNs wherein each of the ssODNs comprises a sequence that is complementary to and specific for a sequence flanking a strand break at an off-target site for a nucleic acid-guided nuclease complex comprising a nucleic acid-guided nuclease and a guide nucleic acid (gNA) wherein the ssODNs each comprise different sequences for different off-target sites.

2. The composition of claim 0 further comprising the nucleic acid-guided nuclease and gNA.

3. The composition of claim 0, wherein each ssODN further comprises a sequence coding for a wild-type gene at the off-target site.

4. The composition of claim 1, wherein at least 10, 20, 30, 40, 50, 60, 70, 80, 90, 95, 99, or 100% of the ssODNs comprise at least one mutation compared to the wild-type sequence.

5. The composition of claim 0, wherein the mutation comprises a mutation to a PAM, and optionally wherein the mutation to the PAM decreases or eliminates recognition of the off-target site by the nucleic acid-guided nuclease complex.

6-11. (canceled)

12. The composition of claim 1, wherein the nucleic acid-guided nuclease is a Type V-A nuclease.

13. The composition of claim 0 wherein the nucleic acid-guided nuclease is a MAD nuclease, an ART nuclease, or an ABW nuclease.

14-24. (canceled)

25. The composition of claim 1, wherein the gNA comprises
(A) a targeter nucleic acid comprising a targeter stem sequence and a spacer sequence; and
(B) a modulator nucleic acid comprising a modulator stem sequence complementary to the targeter stem sequence, and, optionally, a 5′ sequence.

26. The composition of claim 25, wherein the gNA is an engineered, non-naturally occurring guide nucleic acid.

27. (canceled)

28. The composition of claim 1, wherein the gNA comprises a dual guide nucleic acid, wherein the targeter nucleic acid and the modulator nucleic acid are separate polynucleotides.

29-43. (canceled)

44. A method of cleaving at or near a target nucleic acid sequence which is at or near an on-target site within a target polynucleotide comprising contacting the target polynucleotide with the composition of claim 2, wherein the nucleic acid-guided nuclease complex cleaves at least one strand of the target polynucleotide within the on-target site.

45. A method of editing a genome of a eukaryotic cell comprising delivering the composition of claim 2 into the eukaryotic cell, thereby resulting in editing of the genome of the eukaryotic cell.

46-51. (canceled)

52. A composition comprising
(A) a nucleic acid-guided nuclease complex comprising a Type V nuclease and a compatible gNA wherein the nucleic acid-guided nuclease complex specifically binds to a target nucleic acid sequence at or near an on-target site and cleaves at or near the target nucleic acid sequence to create a strand break in the on-target site; and
(B) a first ssODN.

53. The composition of claim 0, wherein the first ssODN comprises a sequence that is complementary to a sequence flanking the strand break in the on-target site on the 3′ side or the 5′ side of the strand break.

54. (canceled)

55. The composition of claim 0, further comprising a second ssODN comprising a sequence that is complementary to a sequence flanking the strand break in the on-target site on the 5′ side or the 3′ side of the strand break.

56-61. (canceled)

62. The composition of claim 52, further comprising one or more ssODNs that are complementary to a sequence flanking the strand break in the one or more off-target sites.

63-69. (canceled)

70. The composition of claim 52, wherein the nuclease is a Type V-A nuclease.

71. The composition of claim 52, wherein the nucleic acid-guided nuclease is a MAD nuclease, an ART nuclease, or an ABW nuclease.

72-82. (canceled)

83. The composition of claim 52, wherein the gNA comprises
(A) a targeter nucleic acid comprising a targeter stem sequence and a spacer sequence; and
(B) a modulator nucleic acid comprising a modulator stem sequence complementary to the targeter stem sequence, and, optionally, a 5′ sequence.

84-85. (canceled)

86. The composition of claim 83, wherein the gNA comprises a dual guide nucleic acid, wherein the targeter nucleic acid and the modulator nucleic acid are separate polynucleotides.

87-119. (canceled)

120. A composition for integrating at least a portion of a donor template at or near a strand break at an on-target or off-target site in a genome of a cell comprising
(A) a donor template lacking one or both homology arms complementary to a sequence or sequences flanking the strand break; and
(B) a first ssODN comprising
(i) a first portion comprising a sequence complementary to at least a 5′ or 3′ portion of the donor template, and
(ii) a second portion comprising a sequence homologous to a sequence flanking the strand break.

121. The composition of claim 0 further comprising:
(C) a second ssODN comprising
(i) a first portion comprising a sequence complementary to at least a 5′ or 3′ portion of the donor template different from the first ssODN, and
(ii) a second portion comprising a sequence homologous to a sequence flanking the strand break.

122. A method for integrating at least a portion of a donor template at a strand break in a target site in a genome of a cell comprising delivering to a cell a composition comprising
(A) the composition of claim 120 to the target cell; and
(B) a nucleic acid guided nuclease complex comprising a nucleic acid-guided nuclease and a compatible gNA, wherein the complex is capable of producing the strand break.

123. (canceled)

124. A composition comprising a plurality of ssODNs comprising
(A) a first ssODN comprising
(i) a first portion comprising a sequence homologous to a sequence upstream of a target site in a genome of a target cell, and
(ii) a second portion comprising a sequence comprising at least a portion of a heterologous sequence to be inserted into the genome of the target cell;
(B) a second ssODN comprising
(i) a first portion comprising a sequence homologous to a sequence downstream of a target site in a genome of a target cell, and
(ii) a second portion comprising a sequence at least partially complementary to at least a portion of the heterologous sequence to be inserted into the genome of the target cell; and, optionally,
(C) one or more additional ssODNs each comprising
(i) a sequence comprising at least a portion of a heterologous sequence to be inserted into the genome of the target cell, and
(ii) a second portion comprising a sequence at least partially complementary to at least a portion of the heterologous sequence to be inserted into the genome of the target cell;
wherein the plurality of ssODNs comprises the entirety of heterologous sequence to be inserted into the genome of the target cell.

125. A method for inserting a heterologous sequence at or near a target site in a genome of a cell comprising delivering the composition of claim 0 to the cell and a nucleic acid-guided nuclease complex capable of binding to and cleaving at the target site.

126. (canceled)

127. A method comprising contacting a population of cells with a composition comprising
(A) a nucleic acid-guided nuclease complex comprising a nucleic acid-guided nuclease and a compatible gNA, wherein the complex can bind to and cleave at an on-target site and one or more off-target sites in the genomes of the cells in the population of cells,
(B) a ssODN, and
(C) one or more ssODNs for one or more of the off-target sites.

128-130. (canceled)

131. A composition comprising
(A) a guide RNA (gRNA) comprising
(i) a first nucleotide sequence that hybridizes to a target nucleic acid sequence in a genome of a cell, and
(ii) a second nucleotide sequence that interacts with a Cas nuclease;
(B) the Cas nuclease, comprising an RNA-binding portion that interacts with the second nucleotide sequence of the guide RNA to form a ribonucleoprotein (RNP) complex, wherein the RNP complex
(i) specifically binds to the target nucleic acid sequence at an on-target site and cleaves at or near the target nucleic acid sequence to create a double-stranded break in the on-target site, and
(ii) also binds to one or more off-target nucleic acid sequences at one or more off-target sites and cleaves at or near the one or more off-target nucleic acid sequences to create a double-strand break in the one or more off-target sites;
(C) a first, on-target ssODN comprising a sequence complementary to a sequence flanking the double stranded break in the on-target site, wherein the ssODN integrates into DNA in the on-target site; and
(D) a second, off-target ssODN comprising a sequence complementary to a genomic sequence flanking a double stranded break in a first off-target site and integrates into the DNA in the off-target site, wherein the second ssODN comprises
(i) homology arms for the off-target site that are more complementary to the genomic sequence at the off-target site than homology arms of the on-target ssODN.

132. (canceled)

133. The composition of claim 0 wherein the second ssODN further comprises at least one synonymous mutation to reduce or eliminate re-cleavage at the off-target site following integration of the second ssODN.

134-137. (canceled)

138. The composition of claim 0 wherein gRNA is dual gRNA.

139-141. (canceled)

142. The composition of claim 131, wherein the Cas nuclease is a type V-A Cas nuclease, optionally wherein the Type V-A Cas nuclease is a Cpf1, MAD, Csm1, ART, or ABW nuclease, or derivative or variant thereof.

143. (canceled)
